SEQUENCE LISTING (33 sequences)

SEQ ID NO: 1 - DNA, 18 bp, Homo sapiens
  misc_feature (1)..(5): n is a, c, g, or t
nnnnnttcnngaannnnn

SEQ ID NO: 2 - DNA, 18 bp, Homo sapiens
  misc_feature (1)..(5): n is a, c, g, or t
nnnnnttctagaannnnn

SEQ ID NO: 3 - DNA, 18 bp, Homo sapiens
  misc_feature (1)..(5): n is a, c, g, or t
nnnnnttccggaannnnn

SEQ ID NO: 4 - RNA, 19 nt, Homo sapiens
ccacuuggaugcuauggac

SEQ ID NO: 5 - RNA, 19 nt, Homo sapiens
agagagacgacacggaguu

SEQ ID NO: 6 - DNA, 20 bp, Artificial
  oligonucleotides
gagccgtaggtcccttcttt

SEQ ID NO: 7 - DNA, 20 bp, Artificial
  oligonucleotides
ctcaggaaccttccagacca

SEQ ID NO: 8 - DNA, 20 bp, Artificial
  oligonucleotides
accgactacgtcatcaccaa

SEQ ID NO: 9 - DNA, 20 bp, Artificial
  oligonucleotides
gtggaaagttccaggacacg

SEQ ID NO: 10 - DNA, 20 bp, Artificial
  oligonucleotides
ttggttgcgcttttagcttt

SEQ ID NO: 11 - DNA, 20 bp, Artificial
  oligonucleotides
accccaagtcacagagatgg

SEQ ID NO: 12 - DNA, 19 bp, Artificial
  oligonucleotides
tttgcccacaaatggacac

SEQ ID NO: 13 - DNA, 20 bp, Artificial
  oligonucleotides
cccaaagaccagctctaacg

SEQ ID NO: 14 - DNA, 20 bp, Artificial
  oligonucleotides
tctcgcacattcttcacgtc

SEQ ID NO: 15 - DNA, 20 bp, Artificial
  oligonucleotides
aggaaccttcccgacttagg

SEQ ID NO: 16 - DNA, 20 bp, Artificial
  oligonucleotides
ttggggtttctcaccagttc

SEQ ID NO: 17 - DNA, 20 bp, Artificial
  oligonucleotides
ctgcagtgctgcttttcttg

SEQ ID NO: 18 - DNA, 19 bp, Artificial
  oligonucleotides
gatctgcccgaaccttctc

SEQ ID NO: 19 - DNA, 19 bp, Artificial
  oligonucleotides
aactttcgcgaacctttcc

SEQ ID NO: 20 - DNA, 20 bp, Artificial
  oligonucleotides
ccaccctgcctcttataccc

SEQ ID NO: 21 - DNA, 20 bp, Artificial
  oligonucleotides
ggcttgtgattgggtcttgt

SEQ ID NO: 22 - DNA, 19 bp, Artificial
  oligonucleotides
cggccggcttagtctagtt

SEQ ID NO: 23 - DNA, 20 bp, Artificial
  oligonucleotides
atttgacccttgagccgtag

SEQ ID NO: 24 - DNA, 20 bp, Artificial
  oligonucleotides
tgagtcatatgggtgtgctg

SEQ ID NO: 25 - DNA, 20 bp, Artificial
  oligonucleotides
tccccttagcacagaagtga

SEQ ID NO: 26 - DNA, 20 bp, Artificial
  oligonucleotides
gggtggaattacagcctcag

SEQ ID NO: 27 - DNA, 20 bp, Artificial
  oligonucleotides
tcctgtagctcccacacctc

SEQ ID NO: 28 - DNA, 18 bp, Artificial
  oligonucleotides
acctggtcggctgcacct

SEQ ID NO: 29 - DNA, 18 bp, Artificial
  oligonucleotides
ttgccctgccatgtctcg

SEQ ID NO: 30 - DNA, 20 bp, Artificial
  oligonucleotides
atgtcaggcccatgaacgat

SEQ ID NO: 31 - DNA, 22 bp, Artificial
  oligonucleotides
gcattcatggagtccaggcttt

SEQ ID NO: 32 - RNA, 19 nt, Homo sapiens
uagccugccuggacaagaa

SEQ ID NO: 33 - RNA, 19 nt, Homo sapiens
gagugaagacauaaagauc

(19) United States
(12) Patent Application Publication
(10) Pub. No.: US 2015/0241436 A1
(43) Pub. Date: Aug. 27, 2015

(54) HSF1 AND HSF1 CANCER SIGNATURE SET GENES AND USES RELATING THERETO

(71) Applicants: Whitehead Institute for Biomedical Research, Cambridge, MA (US); The Brigham and Women's Hospital, Inc., Boston, MA (US)

(72) Inventors: Sandro Santagata, Jamacia Plain, MA (US); Susan Lindquist, Chestnut Hill, MA (US); Marc Mendillo, Cambridge, MA (US); Luke J. Whitesell, Somerville, MA (US)

(21) Appl. No.: 14/398,700

(22) PCT Filed: May 3, 2013
(86) PCT No.: PCT/US2013/039527

§ 371 Date: Nov. 3, 2014

Related U.S. Application Data
(60) Provisional application No. 61/642,394, filed on May 3, 2012.
(60) Provisional application No. 61/656,343, filed on Jun. 6, 2012.

(51) Int. Cl. G01N 33/574

(52) U.S. Cl. CPC G01N 33/57496; G01N 2333/4703

(57) ABSTRACT

In some aspects, the invention relates to Heat Shock Protein-1 (HSF1) gene and HSF1 gene products. In some aspects, the invention provides methods of tumor diagnosis, prognosis, treatment-specific prediction, or treatment selection, the methods comprising assessing the level of HSF1 expression or HSF1 activation in a sample obtained from the tumor. In some aspects, the invention relates to the discovery that increased HSF1 expression and increased HSF1 activation correlate with poor outcome in cancer, e.g., breast cancer. In some aspects, the invention relates to the HSF1 cancer program genes, HSF1 cancer signature set genes, subsets thereof, and uses in tumor diagnosis, prognosis, treatment-specific prediction, treatment selection, or drug discovery, among others.

### CROSS-REFERENCE TO RELATED APPLICATION

This application claims the benefit of U.S. Provisional Application No. 61/642,394, filed May 3, 2012, and U.S. Provisional Application No. 61/656,343, filed Jun. 6, 2012. The entire teachings of the above applications are incorporated herein by reference.

### GOVERNMENT FUNDING STATEMENT

The invention was made with government support under R01-CA146445-01 awarded by the National Cancer Institute, W81XWH-08-1-0282 BC-07456 awarded by the Department of Defense, and K08NS064168 awarded by the National Institutes of Health. The government has certain rights in the invention.

### BACKGROUND OF THE INVENTION

Cancer is a leading cause of death worldwide and accounted for approximately 7.6 million deaths (around 13% of all deaths) in 2008 (Ferlay J, et al., GLOBOCAN 2008 v1.2, Cancer Incidence and Mortality Worldwide: IARC CancerBase No. 10 [Internet]. Lyon, France: International Agency for Research on Cancer; 2010). Although significant progress in the treatment of certain types of cancer such as childhood leukemia has been achieved over the past several decades, many of the most common types of cancer remain difficult to manage and are often incurable, particularly if discovered after the tumor has invaded locally or metastasized. Tumors can exhibit marked variability in terms of aggressiveness and response to treatment, despite displaying similar histopathologic features and stage. Such variability can complicate development of appropriate treatment plans for individual patients. There is a need in the art for identification and elucidation of pathways and cellular changes that contribute to malignancy. There is also a need in the art for innovative approaches for tumor prognosis and for selecting appropriate treatment regimens for individuals with cancer.

### SUMMARY OF THE INVENTION

In some aspects, the invention provides a method of diagnosing cancer in a subject comprising the steps of: determining the level of Heat Shock Factor-1 (HSF1) expression or the level of HSF1 activation in a sample obtained from the subject, wherein increased HSF1 expression or increased HSF1 activation in the sample is indicative that the subject has cancer. In some embodiments, the method comprises comparing the level of HSF1 gene expression or HSF1 activation in the sample with a control level of HSF1 gene expression or HSF1 activation, wherein a greater level in the sample as compared with the control level is indicative that the subject has cancer. In some embodiments, the cancer is a cancer in situ (CIS). In some embodiments, the sample does not show evidence of invasive cancer. In some embodiments the sample comprises breast, lung, colon, prostate tissue, cervical, or nerve sheath tissue. In some embodiments the sample comprises breast tissue and the cancer is ductal carcinoma in situ (DCIS).

In some aspects, the invention provides a method of identifying cancer comprising the steps of: (a) providing a biological sample; and (b) determining the level of HSF1 expression or the level of HSF1 activation in the sample, wherein increased HSF1 expression or increased HSF1 activation in the sample is indicative of cancer. In some embodiments the method comprises comparing the level of HSF1 gene expression or HSF1 activation in the sample with a control level of HSF1 gene expression or HSF1 activation, wherein a greater level in the sample as compared with the control level is indicative of cancer. In some embodiments the sample does not show evidence of invasive cancer. In some embodiments the sample comprises breast, lung, colon, prostate, cervical, or nerve sheath tissue. In some embodiments the sample comprises breast tissue and the cancer is ductal carcinoma in situ (DCIS).

In some aspects, the invention provides a method of assessing a tumor with respect to aggressiveness, the method comprising: determining the level of HSF1 expression or HSF1 activation in a sample obtained from the tumor, wherein an increased level of HSF1 expression or activation is correlated with increased aggressiveness, thereby classifying the tumor with respect to aggressiveness. In some embodiments, the method comprises: (a) determining the level of HSF1 expression or the level of HSF1 activation in a sample obtained from the tumor; (b) comparing the level of HSF1 expression or HSF1 activation with a control level of HSF1 gene expression or HSF1 activation; and (c) assessing the aggressiveness of the tumor based at least in part on the result of step (b), wherein a greater level of HSF1 gene expression or HSF activation in the sample obtained from the tumor as compared with the control level of HSF1 gene expression or HSF activation, respectively, is indicative of increased aggressiveness.

In some aspects, the invention provides a method of classifying a tumor according to predicted outcome comprising steps of: determining the level of HSF1 expression or HSF1 activation in a sample obtained from the tumor, wherein an increased level of HSF1 expression or activation is correlated with poor outcome, thereby classifying the tumor with respect to predicted outcome. In some embodiments the method comprises (a) determining the level of HSF1 expression or the level of HSF1 activation in a tumor sample; and (b) comparing the level of HSF1 expression or HSF1 activation with a control level of HSF1 expression or HSF1 activation, wherein if the level determined in (a) is greater than the control level, the tumor is classified as having an increased likelihood of resulting in a poor outcome.

In some aspects, the invention provides a method of predicting cancer outcome in a subject, the method comprising: determining the level of HSF1 gene expression or the level of HSF1 activation in a tumor sample, wherein an increased level of HSF1 expression or activation is correlated with poor outcome, thereby providing a prediction of cancer outcome. In some embodiments the method comprises: (a) determining the level of HSF1 expression or the level of HSF1 activation in the tumor sample; and (b) comparing the level of HSF1 gene expression or HSF1 activation with a control level of HSF1 gene expression or HSF1 activation, wherein if the level determined in (a) is greater than the control level, the subject has increased likelihood of having a poor outcome.

In some aspects, the invention provides a method for providing prognostic information relating to a tumor, the method comprising: determining the level of HSF1 expression or HSF1 activation in a tumor sample from a subject in need of tumor prognosis, wherein if the level of HSF1 expression or HSF1 activation is increased, the subject is considered to have a poor prognosis. In some embodiments the method comprises: (a) determining the level of HSF1 expression or HSF1 activation in the sample; and (b) comparing the level with a control level, wherein if the level determined in (a) is greater than the control level, the subject is considered to have a poor prognosis.

In some aspects, the invention provides a method for providing treatment-specific predictive information relating to a tumor, the method comprising: determining the level of HSF1 expression or HSF1 activation in a tumor sample from a subject in need of treatment-specific predictive information, wherein the level of HSF1 expression or HSF1 activation correlates with tumor sensitivity or resistance to a treatment, thereby providing treatment-specific predictive information. In some embodiments the treatment comprises hormonal therapy, and the method comprises steps of: (a) determining the level of HSF1 expression or HSF1 activation in the sample; and (b) comparing the level with a control level, wherein if the level determined in (a) is greater than the control level, the tumor has an increased likelihood of being resistant to hormonal therapy. In some embodiments, the treatment comprises proteostasis modulator therapy, method comprising steps of: (a) determining the level of HSF1 expression or HSF1 activation in the sample; and (b) comparing the level with a control level, wherein if the level determined in (a) is greater than the control level, the tumor has an increased likelihood of being sensitive to proteostasis modulator therapy. In some embodiments proteostasis modulator therapy comprises a heat shock response (HSR) inhibitor. In some embodiments proteostasis modulator therapy comprises an HSF1 inhibitor. In some embodiments proteostasis modulator therapy comprises an HSP90 inhibitor. In some embodiments proteostasis modulator therapy comprises a proteasome inhibitor.

In some aspects, the invention provides a method of determining whether a subject with a tumor is a suitable candidate for treatment with a proteostasis modulator, the method comprising assessing the level of HSF1 expression or HSF1 activation in a tumor sample obtained from the subject, wherein an increased level of HSF1 expression or an increased level of HSF1 activation in the sample is indicative that the subject is a suitable candidate for treatment with a proteostasis modulator. In some embodiments the proteostasis modulator is an HSR inhibitor. In some embodiments the proteostasis modulator is an HSF1 inhibitor. In some embodiments, the proteostasis modulator is an HSP90 inhibitor. In some embodiments the proteostasis modulator is a proteasome inhibitor.

In some aspects, the invention provides a method of predicting the likelihood that a tumor will be sensitive to a protein homeostasis modulator, the method comprising: (a) determining the level of HSF1 gene expression or the level of HSF1 activation in a sample obtained from the tumor; and (b) comparing the level of HSF1 gene expression or HSF1 activation with a control level of HSF1 gene expression or HSF1 activation, wherein if the level determined in (a) is greater than the control level, the tumor has an increased likelihood of being sensitive to the protein homeostasis modulator. In some embodiments the proteostasis modulator is an HSR inhibitor. In some embodiments the proteostasis modulator is an HSF1 inhibitor. In some embodiments, the proteostasis modulator is an HSP90 inhibitor. In some embodiments the proteostasis modulator is a proteasome inhibitor. In some embodiments the tumor is a carcinoma, e.g., an adenocarcinoma. In some embodiments the tumor is a CIS. In some embodiments the tumor is a Stage I tumor. In some embodiments the tumor is a breast, lung, colon, prostate, cervical, or malignant nerve sheath tumor. In some embodiments the tumor is a stage I lung adenocarcinoma or stage I breast tumor. In certain embodiments the tumor is a breast tumor, e.g., a breast tumor that is positive for estrogen receptor (ER) positive breast tumor, human epidermal growth factor 2 (HER2), or both. In some embodiments the tumor is a lymph node negative tumor, e.g., a lymph node negative breast tumor. In certain embodiments the tumor is a ductal carcinoma in situ (DCIS). In certain embodiments in which the tumor is a breast tumor, the method further comprises assessing the sample for ER, progesterone receptor (PR), HER2 status, or lymph node status (or any combination thereof).

In some aspects, the invention provides a method for tumor diagnosis, prognosis, treatment-specific prediction, or treatment selection comprising: (a) providing a sample obtained from a subject in need of diagnosis, prognosis, treatment-specific prediction, or treatment selection for a tumor; (b) determining the level of HSF1 expression or HSF1 activation in the sample; (c) scoring the sample based on the level of HSF1 expression or HSF1 activation, wherein the score provides diagnostic, prognostic, treatment-specific predictive, or treatment selection information. In some embodiments, scoring comprises determining the level of an HSF1 gene product in the sample. In some embodiments, scoring comprises determining the level of HSF1 in nuclei of cells in the sample. In some embodiments, scoring comprises generating a composite score based on the percentage of cells that exhibit nuclear HSF1 and the level of nuclear HSF1. In some embodiments, scoring comprises comparing the level of HSF1 expression or HSF1 activation in the sample with the level of HSF1 expression or HSF1 activation in a control. In some embodiments the tumor is a carcinoma, e.g., an adenocarcinoma. In some embodiments the tumor is a sarcoma. In some embodiments the tumor is a CIS. In some embodiments the tumor is a stage I tumor. In some embodiments the tumor is a breast, lung, colon, prostate, cervical, or malignant nerve sheath tumor. In some embodiments the tumor is a stage I lung adenocarcinoma or stage breast tumor. In certain embodiments the tumor is a breast tumor, e.g., a breast tumor that is positive for estrogen receptor (ER) positive breast tumor, human epidermal growth factor 2 (HER2), or both. In some embodiments the tumor is a lymph node negative tumor, e.g., a lymph node negative breast tumor. In certain embodiments the tumor is a ductal carcinoma in situ (DCIS). In certain embodiments the tumor is an ER positive, lymph node negative breast tumor. In some embodiments wherein the tumor is a breast tumor and the method further comprises scoring the tumor for ER, PR, HER2, or lymph node status.

In some embodiments of any of the methods, determining the level of HSF1 expression comprises determining the level of an HSF1 gene product.

In some embodiments of any of the methods, determining the level of HSF1 expression comprises determining the level of HSF1 mRNA.

In some embodiments of any of the methods, determining the level of HSF1 expression comprises determining the level of HSF1 polypeptide.

In some embodiments of any of the methods, determining the level of HSF1 expression comprises detecting HSF1 polypeptide using an antibody that binds to HSF1 polypeptide.

In some embodiments of any of the methods, the sample comprises a tissue sample, and determining the level of expression or activation of HSF1 comprises performing immunohistochemistry (IHC) on the tissue sample.

In some embodiments of any of the methods, determining the level of HSF1 activation comprises measuring at least one bioactivity of HSF1 protein.

In some embodiments of any of the methods, determining the level of HSF1 activation comprises determining the localization of HSF1 polypeptide in cells, wherein nuclear localization is indicative of HSF1 activation. In some embodiments, nuclear localization is assessed using IHC.

In some embodiments of any of the methods, determining the level of HSF1 activation comprises detecting at least one post-translational modification of HSF1 polypeptide.

In some embodiments of any of the methods, determining the level of HSF1 activation comprises determining the level of phosphorylation of HSF1 polypeptide on serine 326, wherein phosphorylation of HSF1 polypeptide on serine 326 is indicative of HSF1 activation. In some embodiments the level of phosphorylated HSF1 (e.g., HSF1 phosphorylated on serine 326), is determined using an antibody that binds specifically to phosphorylated HSF1.

In some embodiments of any of the methods, determining the level of HSF1 activation comprises determining the level of chromatin occupancy by HSF1 polypeptide.

In some embodiments of any of the methods, determining the level of HSF1 activation comprises determining the level of a gene expression product of at least one HSF1-regulated gene other than a heat shock protein (HSP) gene.

In some aspects, the invention relates to identification of a transcriptional program regulated by HSF1 in cancer cells. In some aspects, the invention provides HSF1 cancer program (HSF1-CP) genes and subsets thereof. In some aspects, the invention provides HSF1 cancer signature set (CSS) genes and subsets thereof. In some aspects, the invention provides HSF1-CaSig, HSF1-CaSig2, HSF1-CaSig3, and refined HSF1-CSS cancer signature sets. In some aspects, the invention provides coordinately regulated sets of genes (Modules 1-5) comprising subsets of the HSF1-CP genes.

In some embodiments of any of the methods comprising determining the level of HSF1 activation, such determining comprises assessing expression of at least one HSF1 cancer program (HSF1-CP) gene. In some embodiments determining the level of HSF1 activation comprises determining the level of a gene product of at least one HSF1-CP gene. In some embodiments determining the level of HSF1 activation comprises assessing expression of an HSF1 cancer signature set (CSS) or subset thereof. In some embodiments determining the level of HSF1 activation comprises determining the level of a gene product of at least one HSF1-CSS gene.

In some embodiments of any of the methods, an HSF1 cancer signature set is HSF1-CaSig, HSF1-CaSig2, HSF1-CaSig3, or a refined HSF1-CSS. In some embodiments of any of the methods, an HSF1 cancer signature set gene is part of HSF1-CaSig, HSF1-CaSig2, HSF1-CaSig3, or a refined HSF1-CSS.

In some aspects, the invention provides a method of diagnosing cancer in a subject comprising: (a) determining a gene expression profile of an HSF1 cancer signature set (HSF1-CSS) or subset thereof in a sample obtained from a subject; and (b) determining whether the sample represents cancer based at least in part on the gene expression profile. In some aspects, the invention provides a method of identifying cancer comprising the steps of: (a) providing a biological sample; and (b) determining a gene expression profile of an HSF1 cancer signature set or subset thereof in the sample; and (c) determining whether the sample represents cancer based at least in part on the gene expression profile. In some embodiments, a method of diagnosing cancer or identifying cancer comprises determining whether the gene expression profile clusters with gene expression profiles representative of cancer or whether the gene expression profile clusters with gene expression profiles representative of non-cancer. In some embodiments the method comprises determining whether expression of the HSF1-CSS falls into a high or low expression subset, wherein high expression is indicative of cancer.

In some aspects, the invention provides a method of assessing a tumor with respect to aggressiveness, the method comprising: (a) determining a gene expression profile of an HSF1 cancer signature set or subset thereof in a sample obtained from a subject; and (b) determining whether the sample represents an aggressive cancer based at least in part on the gene expression profile, thereby classifying the tumor with respect to aggressiveness. In some embodiments the level of HSF1-CSS expression is compared with a control. In some embodiments an increased level of HSF1-CSS expression as compared with a control is indicative of increased aggressiveness. In some embodiments, the method comprises determining whether the gene expression profile clusters with gene expression profiles representative of aggressive cancer or whether the gene expression profile clusters with gene expression profiles representative of non-aggressive cancer or non-cancer. In some embodiments the method comprises determining whether expression of the HSF1-CSS falls into a high or low expression subset, wherein high expression is indicative of aggressive cancer.

In some aspects, the invention provides a method of classifying a tumor according to predicted outcome comprising steps of: (a) determining a gene expression profile of an HSF1 cancer signature set or subset thereof in a sample obtained from a subject; and (b) classifying the tumor with respect to predicted outcome based at least in part on the gene expression profile. In some embodiments the level of HSF1-CSS expression is compared with a control. In some embodiments an increased level of HSF1-CSS expression as compared with a control is indicative of increased likelihood of poor outcome. In some aspects, the invention provides a method for providing prognostic information relating to a tumor, the method comprising: (a) determining a gene expression profile of an HSF1 cancer signature set or subset thereof in a tumor sample obtained from a subject in need of tumor prognosis; and (b) determining a prognosis based at least in part on the gene expression profile. In some embodiments the level of HSF1-CSS expression is compared with a control. In some embodiments an increased level of HSF1-CSS expression as compared with a control is indicative of a poor prognosis. In some embodiments the level of HSF1-CSS expression is compared with a control. In some embodiments an increased level of HSF1-CSS expression as compared with a control is indicative of increased likelihood of poor outcome, or poor prognosis. In some embodiments, the method comprises determining whether the gene expression profile clusters with gene expression profiles representative of cancers with a poor outcome, or poor prognosis or whether the gene expression profile clusters with gene expression profiles representative of cancers with a good outcome, or good prognosis. In some embodiments the method comprises determining whether expression of the HSF1-CSS genes falls into a high or low expression subset, wherein high expression is indicative of cancer with an increased likelihood of poor outcome (poor prognosis).

In some aspects, the invention provides a method for providing treatment-specific predictive information relating to a tumor, comprising: (a) determining a gene expression profile of an HSF1 cancer signature set or subset thereof in a tumor sample from a subject in need of treatment-specific predictive information for a tumor, wherein the gene expression profile correlates with tumor sensitivity or resistance to a treatment, thereby providing treatment-specific predictive information. In some embodiments, the method comprises determining whether the gene expression profile clusters with gene expression profiles representative of cancers that are sensitive or resistant to a treatment.

In some aspects, the invention provides a method for tumor diagnosis, prognosis, treatment-specific prediction, or treatment selection comprising: (a) providing a sample obtained from a subject in need of diagnosis, prognosis, treatment-specific prediction, or treatment selection for a tumor; (b) determining a gene expression profile of an HSF1 cancer signature set or subset thereof in in the sample; (c) scoring the sample based on the gene expression profile, wherein the score provides diagnostic, prognostic, treatment-specific predictive, or treatment selection information. In some embodiments, the method comprises determining whether the gene expression profile clusters with gene expression profiles representative of cancers having a selected prognosis, outcome, or likelihood of treatment response. In some embodiments the method comprises determining whether expression of the HSF1-CSS falls into a high or low expression subset.

In some aspects, the invention provides a method of predicting the likelihood that a tumor will be sensitive to a protein homeostasis modulator, the method comprising: (a) determining a gene expression profile of an HSF1 cancer signature set or subset thereof in a tumor sample obtained from a subject in need of treatment for cancer; and (b) predicting the likelihood that a tumor will be sensitive to a protein homeostasis modulator based at least in part on the gene expression profile. In some embodiments the level of HSF1-CSS expression is compared with a control. In some embodiments an increased level of HSF1-CSS expression as compared with a control is indicative that the tumor has an increased likelihood of being sensitive to the protein homeostasis modulator. In some aspects, the invention provides a method of determining whether a subject with a tumor is a suitable candidate for treatment with a proteostasis modulator, comprising (a) determining a gene expression profile of an HSF1 cancer signature set or subset thereof in a tumor sample obtained from a subject in need of treatment for cancer; and (b) predicting the likelihood that a tumor will be sensitive to a proteostasis modulator based at least in part on the gene expression profile, wherein if the tumor is likely to be sensitive to the proteostasis modulator, the subject is a suitable candidate for treatment with the proteostasis modulator. In some embodiments the level of HSF1-CSS expression is compared with a control. In some embodiments an increased level of HSF1-CSS expression as compared with a control is indicative that the subject is a suitable candidate for treatment with a proteostasis modulator.

In some embodiments a gene expression profile comprises a measurement of expression of at least 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, Group B genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes. In some embodiments a gene expression profile comprises a measurement of expression of at least 1, 2, 3, 4, 5, 10, 15, 20, 25, 30, 40, 50, 60, 70, 80, 90, or 100 HSF1-CP gene whose expression is increased by at least 1.2-fold in cancer cells as compared with non-transformed control cells not subjected to heat shock. In some embodiments an HSF1 cancer signature set is HSF1-CaSig, HSF1-CaSig2, HSF1-CaSig3 gene, or a refined HSF1-CSS. In some embodiments an HSF1 cancer signature set comprises or is composed of genes listed in Table T4C, Table T4D, Table T4E, or Table T4F. In some embodiments at least 70%, 80%, 90%, 95%, or more (e.g., 100%) of the genes in an HSF1-CSS or subset thereof are positively regulated by HSF1 in cancer cells. In some embodiments expression of at least 70%, 80%, 90%, 95%, or more (e.g., 100%) of the genes in an HSF1-CSS are positively correlated with poor prognosis. In some embodiments, expression of a gene is positively weighted if its expression is positively correlated with an outcome or characteristic of interest (e.g., poor prognosis) and negatively weighted if its expression is negatively correlated with an outcome or characteristic of interest. In some embodiments, expression of a gene is positively weighted if its regulation by HSF1 is positively correlated with an outcome or characteristic of interest (e.g., poor prognosis) and negatively weighted if its regulation by HSF1 is negatively correlated with an outcome or characteristic of interest.

In some aspects, the invention provides a method of identifying a candidate modulator of HSF1 cancer-related activity, the method comprising: (a) providing a cell comprising a nucleic acid construct comprising (i) at least a portion of a regulatory region of an HSF1-CP gene operably linked to a nucleic acid sequence encoding a reporter molecule, wherein the HSF1-CP gene is an HSF1-CP Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF in cancer cells than in heat shocked non-transformed cells; (b) contacting the cell with a test agent; and (c) assessing expression of the nucleic acid sequence encoding the reporter molecule, wherein the test agent is identified as a candidate modulator of HSF1 cancer-related activity if expression of the nucleic acid sequence encoding the reporter molecule differs from a control level. In some embodiments the cell is a cancer cell. In some embodiments assessing expression of the nucleic acid sequence encoding comprises measuring the level or activity of the reporter molecule. In some embodiments the portion of a regulatory region comprises a HSE and a YY1 element. In some embodiments the portion of a regulatory region comprises a YY1 binding site and a HSE comprising exactly 3 inverted repeat units. In some embodiments the test agent is identified as a candidate inhibitor of HSF1 cancer-related activity if expression of the nucleic acid sequence encoding the reporter molecule is reduced as compared with the control level. In some embodiments the method further comprises assessing the effect of the test agent on expression of one or more HSF1-CP genes. In some embodiments the method further comprises assessing the effect of the test agent on a gene expression profile of an HSF1 cancer signature set or subset thereof. In some embodiments, if the test agent modulates expression of the one or more HSF1-CP genes or HSF1 cancer signature set, the test agent is confirmed as a candidate modulator of HSF1 cancer-related activity.

In some aspects, the invention provides a method of identifying a candidate modulator of HSF1 cancer-related activity comprising steps of: (a) contacting a cell that expresses HSF1 with a test agent; (b) measuring the level of an HSF1 cancer-related activity exhibited by the cell; and (c) determining whether the test agent modulates the HSF1 cancer-related activity, wherein a difference in the level of the HSF1 cancer-related activity in the presence of the test agent as compared to the level in the absence of the test agent identifies the agent as a candidate modulator of HSF1 cancer-related activity. In some embodiments measuring the level of an HSF cancer-related activity comprises measuring binding of HSF1 to a regulatory region of an HSF1-CP gene, Group A gene, HSF1-CSS gene, HSF1-CaSig2 gene, HSF1-CaSig3 gene, refined HSF1-CSS gene, Module 1 gene, Module 2 gene, Module 3 gene, Module 4 gene, or Module 5 gene or measuring expression of an HSF1-CP gene, Group A gene, Group B gene, HSF1-CSS gene, refined HSF1-CSS gene, Module 1 gene, Module 2 gene, Module 3 gene, Module 4 gene, or Module 5 gene, wherein the gene is more highly bound by HSF1 in cancer cells than in heat shocked non-transformed control cells. In some embodiments measuring the level of an HSF cancer-related activity comprises measuring binding of HSF1 to the regulatory regions of at least 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes or measuring expression of at least 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, Group B genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes, wherein at least one of the genes is more highly bound by HSF1 in cancer cells than in heat shocked non-transformed control cells.

In some aspects, the invention provides a method of identifying a candidate modulator of HSF1 cancer-related activity, the method comprising: (a) providing a cell comprising a nucleic acid construct comprising (i) at least a portion of a regulatory region of an HSF1-CP gene operably linked to a nucleic acid sequence encoding a reporter molecule, wherein the HSF1-CP gene is an HSF1-CP Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed cells; (b) contacting the cell with a test agent; and (c) assessing expression of the nucleic acid sequence encoding the reporter molecule, wherein the test agent is identified as a candidate modulator of HSF1 cancer-related activity if expression of the nucleic acid sequence encoding the reporter molecule differs from a control level.

In some aspects, the invention provides an isolated nucleic acid comprising at least one YY1 binding site and a heat shock element (HSE). In some embodiments the invention provides a nucleic acid construct comprising the isolated nucleic acid and a sequence encoding a reporter molecule. In some embodiments the sequence of an isolated nucleic acid comprises at least a portion of a regulatory region of a Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed control cells. Further provided are vectors and cells comprising the isolated nucleic acid or nucleic acid construct. Further provided are methods of using the isolated nucleic acid, nucleic acid construct, vector, or cell, e.g., in identification of candidate modulators of HSF1 cancer-related activity.

In some embodiments of any aspect herein, a tumor is a breast, lung, colon, prostate, pancreas, cervical, or nerve sheath tumor. In some embodiments a tumor is breast, lung, or colon tumor. In some embodiments a tumor is a breast tumor. In some embodiments a tumor is an estrogen receptor (ER) positive breast tumor. In some embodiments a tumor is a human epidermal growth factor 2 (HER2) positive breast tumor. In some embodiments a tumor is a lymph node negative breast tumor. In some embodiments a tumor is an estrogen receptor (ER) positive, lymph node negative breast tumor.

In various embodiments of the methods described herein, a control sample can comprise normal non-neoplastic cells or tissue, e.g., normal non-neoplastic cells or tissue of the same type or origin as that from which a tumor arose. In various embodiments of the methods described herein, a control level of HSF1 expression or HSF1 activation can be a level measured in normal non-neoplastic cells or tissue, e.g., normal non-neoplastic cells or tissue of the same type or origin as that from which a tumor arose, e.g., as measured under conditions that do not activate the heat shock response.

In some embodiments, any of the methods can comprise providing a sample, e.g., a tumor sample. In some embodiments, any of the method can comprise providing a subject, e.g., a subject in need of tumor diagnosis, prognosis, or treatment selection.

In some embodiments, any of the methods can further comprise assessing at least one additional cancer biomarker. The at least one additional cancer biomarker is typically a gene or gene product (e.g., mRNA or protein) whose expression, activation, localization, or activity, correlates with the presence or absence of cancer, with cancer aggressiveness, with cancer outcome, cancer prognosis, or treatment-specific cancer outcome. The cancer biomarker(s) can be selected, e.g., at least in part based on the tumor type.

In some embodiments, any of the methods can further comprise selecting or administering a therapeutic agent based at least in part on results of assessing the level of HSF1 expression or HSF1 activation. In some aspects, the invention provides a method comprising selecting or administering a treatment to a subject in need of treatment for a tumor, wherein the treatment is selected based at least in part on an assessment of the level of HSF1 expression or HSF1 activation in a sample obtained from the tumor. In some embodiments, a method comprises selecting or administering an appropriate therapy if CIS is detected. For example, the therapy can comprise surgical removal of the CIS. In some embodiments a method comprises selecting or administering a more aggressive therapy if a tumor (or sample obtained therefrom) is classified as having an increased likelihood of being aggressive, if a tumor or subject is classified as having an increased likelihood of having a poor outcome, or if a subject is classified as having a poor prognosis. For example, in some embodiments a method comprises selecting or administering adjuvant therapy (e.g., adjuvant chemotherapy) if a tumor (or sample obtained therefrom) is classified as having an increased likelihood of being aggressive, if a tumor or subject is classified as having an increased likelihood of having a poor outcome, or if a subject is classified as having a poor prognosis. In some embodiments a method comprises selecting or administering a proteostasis modulator if the level of HSF1 expression or the level of HSF1 activation is increased.

In some aspects, the invention provides a kit that comprises at least one agent of use to measure the level of HSF1 expression or HSF1 activation in a sample, e.g., an agent that specifically binds to an HSF1 gene product (e.g., HSF1 mRNA or HSF1 protein). The agent may be, e.g., an antibody, or a nucleic acid. In some embodiments the agent is validated for use in assessing HSF1 expression or HSF1 activation, in that results of an assay using the agent have been shown to correlate with cancer outcome, prognosis, or treatment efficacy of at least one specific treatment. In some embodiments the agent is an antibody useful for performing IHC. In some embodiments the kit comprises a reporter construct suitable for assessing HSF1 cancer-related transcription. In some embodiments the kit comprises a cell comprising a reporter construct suitable for assessing HSF1 cancer-related transcription. In some aspects, the invention provides a kit or collection comprising reagents suitable for assessing expression of at least 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, Group B genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes.

Certain conventional techniques and concepts of cell biology, cell culture, molecular biology, microbiology, recombinant nucleic acid (e.g., DNA) technology, immunology, etc., which are within the skill and knowledge of those of ordinary skill in the art, may be of use in aspects of the invention. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005; Buchwalow, I, and Böcker, W. (2010) Immunohistochemistry: Basics and Methods, Methods in Molecular Medicine, Springer) Lodish H, et al. (2007). Molecular cell biology (6th ed.). New York: W.H. Freeman and CO. Further information on cancer and treatment thereof may be found in Cancer: Principles and Practice of Oncology (V. T. De Vita et al., eds., J. B. Lippincott Company, 8^{th} ed., 2008 or 9^{th} ed., 2011) and Weinberg, R A, The Biology of Cancer, Garland Science, 2006. All patents, patent applications, books, journal articles, databases, websites, and other publications mentioned herein are incorporated herein by reference in their entirety. In the event of a conflict or inconsistency with the specification, the specification shall control. Applicants reserve the right to amend the specification based on any of the incorporated references and/or to correct obvious errors. None of the content of the incorporated references shall limit the invention.

### BRIEF DESCRIPTION OF THE DRAWING

FIG. 1. HSF1 protein is increased in breast cancer. (A) Characterization of HSF1 antibody. Immunoblot analysis of spleen lysates from HSF1 wild-type (+/+) and HSF1 null mice (−/−). (B) Immunohistochemistry of mouse brain from HSF1 wild-type and HSF1 null mice, long development. Scale bar, 20 μM. (C) Upper panel, HSF1 immunoblot of matched pairs of invasive ductal carcinoma and adjacent normal breast from seven patients. Lower panel, protein stain for loading comparison.

FIG. 2. HSF1 is increased and localized to the nucleus in invasive and in situ breast carcinoma. Photomicrographs of H&E sections and HSF1 immunohistochemistry of (A, B) invasive ductal carcinoma and (C, D) the pre-invasive lesion, ductal carcinoma in situ (DCIS). Non-neoplastic breast epithelium is indicated by the arrows and neoplastic cells are indicated by the arrowheads. (E) Representative photomicrographs of tumors from the NHS tissue microarrays that were stained by HSF1 immunohistochemistry and that were scored as having either no (−), low, or high nuclear HSF1 expression. This example with no nuclear HSF1 expression (−) demonstrates weak immunoreactivity in the cytoplasm. Scale bar, 20 μM.

FIG. 3. HSF1-positive tumors are associated with decreased survival in estrogen receptor-positive breast cancer. (A) Kaplan-Meier analysis of all individuals with breast cancer that were scored in this study. Kaplan-Meier analysis of participants with (B) HER2 positive (HER2+) breast cancer, (C) triple-negative breast cancer and (D) estrogen receptor-positive (ER+) breast cancer that had HSF1 in the nucleus (HSF1+) or that had no detectable nuclear FISH (HSF1−). In these analyses, low and high nuclear HSF1 expressors were included in the HSF1+ group. Kaplan-Meier analysis of individuals with (E) ER+, HER2+ and triple-negative breast cancer or (F) with only ER+ breast cancer expressing no nuclear HSF1, low nuclear HSF1 or high nuclear HSF1. Nurses' Health Study (1976-1997). Log-rank p values are shown.

FIG. 4. HSF1 is activated in multiple human breast carcinoma subtypes. (A) High magnification of HSF1 staining in ER+, HER2+ and triple-negative breast sections. (B) HSF1 is translocated from the cytoplasm to the nucleus in transformed cells in human breast tissue. Immunoperoxidase staining (brown) with an anti-HSF1 antibody of formalin-fixed paraffin-embedded human biopsy material containing both tumor and normal cells. Sections were counterstained with hematoxylin to identify nuclei (blue). (C) Representative photomicrographs of tumors from the breast cancer TMAs that were stained by HSF1 immunohistochemistry and that were scored as having weak (white), low (pink), or high (red) HSF1 expression. Scoring for three TMAs are displayed as heatmaps. The top panel contains data from two TMAs, which together contain 138 breast tumors representing all major breast cancer subtypes. ER+ and HER2+ expression, in addition to HSF1 nuclear expression, are displayed. The middle panel displays the HSF1 nuclear expression of a triple-negative breast cancer TMA consisting of 151 tumors. The bottom panel displays the HSF1 nuclear expression of 16 normal mammary tissue sections. A summary of all HSF1 expression by tissue subtype is quantified in the bargraph on the right. (D) HSF1 nuclear protein expression is correlated with poor outcome in ER+, lymph-node negative tumors from NHS.

FIG. 5. HSF1 is activated in multiple human carcinoma types. Immunoperoxidase staining (brown) with an anti-HSF1 antibody of formalin-fixed paraffin-embedded human biopsy material of the indicated tissue types (lung, colon, prostate, breast) showing areas of neoplastic (cancerous) and non-neoplastic (noncancerous) tissue as indicated.

FIG. 6. HSF1 is uniformly expressed in invasive ductal carcinoma cells. (A) Low magnification H&E image of an invasive breast carcinoma. Scale bar, 150 μM. (B) HSF1 immunohistochemistry of the same area of the tumor demonstrates uniform HSF1 expression in invasive ductal carcinoma cells across the tumor cross section. There was no difference in intensity of staining at the center of the tumor versus the outer tumor/stroma interface. HSF1 immunohistochemistry demonstrating uniform HSF1 expression in invasive ductal carcinoma cells (C) embedded in a region of necrosis and (D) independent of adjacent inflammation or blood vessels. The black arrow indicates non-neoplastic breast epithelium. The black arrowhead indicates tumor cells adjacent to small blood vessels (asterisks). The two red arrowheads indicate tumor cells that are embedded in a region with desmoplasia and marked inflammation. These two photomicrographs are from neighboring regions of the same section of tumor. Scale bar, 100 μM.

FIG. 7. HSF1 mRNA levels are associated with poor outcome in breast cancer. Kaplan-Meier analysis of all 295 individuals (A), only ER-positive (B) and only ER-negative patients (C) from Van de Vijver et al. (17). The highest 50% of cases expressing HSF1 constituted the HSF1-high group and the lowest 50% of cases constituted the HSF1-low group. Log-rank p values are shown.

FIG. 8: IHC of HSF1 in additional ER+, HER2+& Triple Negative tumors. Immunoperoxidase staining (brown) with an anti-HSF1 antibody of formalin-fixed paraffin-embedded human biopsy material of (A) normal mammary tissue or (B) the indicated tumor subtypes. Blue staining nuclei with Mayer-hematoxylin counterstain are negative for HSF1. ER+ (estrogen receptor positive); TN (triple negative).

FIG. 9. HSF1 mRNA levels are associated with poor outcome in lung cancer. Kaplan-Meier analysis showing overall survival and disease free progression in a group of 70 stage I lung cancers. ACA=adenocarcinoma

FIG. 10. HSF1 is activated in metastatic and highly tumorigenic human mammary epithelial cell lines. (A) Equal amounts of total cellular protein from the indicated cell lines were immunoblotted with HSF1 (Ab4) or a phospho-S326-HSF1 antibody. ACTB was the loading control. (B) Immunohistochemical staining (brown) with anti-HSF1 antibody (Ab4) of HMLER or BPLER xenograft tumors established in mice. Upper panels show regions of viable tumor (high magnification, scale bar 20 μM) and lower panels show the interface of viable tumor and areas of necrosis (lower magnification, scale bar 5004) (C) Schematic diagram depicting the source for each experimental group analyzed by HSF1 ChIP-Seq (see text for details). (D) Scatter plot of peak heights for each region of HSF1 occupancy identified by ChIP-Seq, normalized by the total number of reads in the dataset generated for each experimental condition. (E) Venn diagram depicting overlap of genes bound in malignant cells (BPLER at 37° C.) and immortalized, non-tumorigenic cells after heat shock (BPE or HME cells at 42° C.). (F) HSF1 binding for representative genes bound strongly in highly malignant BPLER cells (CKS2, LY6K, RBM23) and bound in both BPLER cells and heat-shocked HME and BPE cells (HSPA6, HSPA8, PROM2). Arrows indicate transcription start site of each gene. Y-axis: reads per million total reads. X-axis: from −2 kb from the transcription start site (TSS) to either +5, +6 or +10 kb from the (TSS) for each gene; genes diagrams are drawn to scale.

FIG. 11. The expression of HSF1-bound genes is altered by HSF1 depletion. (A) Relative gene expression levels following shRNA-mediated knockdown of HSF1 in HMLER, BPLER and MCF7 cells, Genes are grouped into those previously shown by ChIP-Seq to be bound only in cancer (BPLER at 37° C.; upper panel) and those bound in cancer (BPLER at 37° C.) and in parental cells (HME and BPE) following heat shock (lower panel). Scr and GFP were negative control shRNA. (B) Bar graph depicting the number of genes positively regulated (reduced expression upon HSF1 depletion) or negatively regulated (increased expression upon HSF1 depletion) by HSF1 relative to site of gene occupancy by HSF1 (promoter versus distal).

FIG. 12. Genome-wide patterns of DNA occupancy by HSF1 across a broad range of common human cancer cell lines. (A) Heat map depicting ChIP-Seq read density for all HSF1 target regions (union of all HSF1-bound regions in all datasets). Genomic regions from −1 kb to +1 kb relative to the peak of HSF1 binding are shown. Regions are ordered the same in all datasets. Read density is depicted for non-tumorigenic cells at 37° C. (green), cancer cell lines at 37° C. (black) and non-tumorigenic (nt) cells following heat shock at 42° C. (red). Asterisks indicate datasets that were also used for the analysis presented in FIG. 1E. (B) Principal component analysis of HSF1 binding in heat-shocked parental cell lines (red) and cancer cell lines (black), (C) ChIP-Seq density heat map of genomic regions differentially bound by HSF1 in cancer cell lines at 37° C. (black), heat-shocked non-tumorigenic cells (red), and regions shared under both conditions. (D) HSF1 binding of representative genes in cancer cell lines at 37° C. (black: BT20, NCIH838, SKBR3) and heat-shocked non-tumorigenic cells (red: HME, BPE, MCF10A). Examples of genes with distinct patterns of binding are presented: Enriched in cancer cell lines, enriched in heat-shocked non-tumorigenic cells lines, or enriched in both (blue: shared. Arrows denote transcription start site of gene. Reads per million total reads are shown. (E) Motif analysis of the 100 bp genomic regions surrounding HSF1 binding peaks for genes enriched in cancer cells BT20, NCIH838 and SKBR3 (black:cancer enriched).) Analyses of motifs in heat-shocked non-tumorigenic cells HME, BPE, MCF10A (red: heat shock enriched), and motifs enriched in both cancer cell lines and heat-shocked non-tumorigenic cells lines (blue: shared) are also presented.

FIG. 13. Distinct, coordinately-regulated modules of HSF1-bound genes. (A) Graphical representation of the HSF1 cancer program integrating information on gene binding, regulation and function. For each gene depicted, the peak height is reflected in the diameter of the circle (log 2 peak height: range ˜3 to 9). Color intensity reflects extent of gene regulation following shRNA knockdown (average of log 2 fold change in BPLER and MCF7 cells following shRNA knockdown of HSF1; red—positively regulated; green—negatively regulated; gray—no data because a relevant probe was not present on expression array). Genes are clustered by broad functional categories (gray balloons). (B) Gene-gene expression correlation matrix of HSF1-bound genes. Pair-wise correlation map is presented of the genes that were bound by HSF1 in at least two of the three cancer cell lines (BT20, NCIH38, and SKBR3). The Pearson correlation coefficient (r; between +0.7 (yellow) and −0.7 (blue)) relating normalized mRNA expression data for each gene pair was assessed in nearly 12,000 expression profiles from the Celsius database using the UCLA Gene Expression Tool (UGET). Enriched GO (gene-ontology) categories for each module are shown.

FIG. 14. HSF1 is activated in a broad range of human tumors. (A) Immunohistochemistry (IHC) demonstrates high level nuclear staining for HSF1 in the tumor cells of a human breast cancer specimen (top of panel) with adjacent normal breast epithelial cells (bottom of panel) showing a lack of nuclear HSF1. (B) Representative images of HSF1 IHC performed on breast cancer tissue microarray (TMA) cores. Examples of weak (white), low (pink), or high (red) HSF1 nuclear expression are shown. The scoring of three different TMAs is displayed in heat map format. The top panel depicts data from two TMAs (Mixed Breast Arrays BRC1501 and BRC1502), which together contained 138 breast tumors representing all major breast cancer subtypes. Progesterone receptor (PR), ER, and HER2 were evaluated by IHC as well as HSF1. The middle panel shows relative nuclear HSF1 staining of triple negative breast cancer cases from a TMA consisting of 161 tumors (TN). The bottom panel displays the lack of HSF1 nuclear expression in 16 normal mammary tissue sections. A summary of results for HSF1 staining across all the TMAs is provided in the bar graph (right). (C) Representative images of HSF1 IHC showing high level nuclear staining in a panel of invasive human tumors including carcinomas of the cervix, colon, lung, pancreas, and prostate and in a mesenchymal tumor, meningioma; T, Tumor; N, Normal adjacent tissue. A quantitative summary of all HSF1 IHC results categorized by tissue type from an analysis of TMAs or whole tissue sections is presented in the bar graph (right). (D) ChIP-Seq analysis of human breast and colon cancer specimens. Heat map depicting ChIP-Seq read density in surgical resection specimens for all HSF1 target regions. For reference, the binding profiles for cancer cell lines in culture (black; average across BT20, NCIH838 and SKBR3) and parental heat-shocked cell lines (red) are included. HSF1 nuclear expression was also evaluated by immunohistochemistry in each of the samples used for ChIP-Seq (see Figure S5C) and scored as in Panel B. (E) HSF1 binding in cell lines compared to resected tumor specimens. Average binding across cancer cell lines in cell culture (black; average across BT20, NCIH838 and SKBR3), parental heat-shocked cell lines (red), and individual patient tumors (cyan) are depicted for the representative target genes indicated. Arrows denote transcription start site of gene. Reads per million total reads are shown. (F) Principal component analysis of HSF1 binding in heat-shocked parental cell lines (red), cancer cells lines (black) and patient tumors (cyan).

FIG. 15. An HSF1-cancer signature is associated with reduced survival in patients with breast cancer. (A) Representative dataset (n=159 tumors; (Pawitan et al., 2005)) is shown from a meta-analysis of 10 publicly available mRNA expression datasets (see Table T5) derived from human breast tumors with known clinical outcome and representing a total of 1594 patients. Each column corresponds to a tumor, and each row corresponds to a microarray probe for an HSF1-cancer signature (HSF1-CaSig) gene. Median levels of expression are depicted in black, increased expression in yellow, and decreased expression in blue. Tumors are ordered by average level of expression of the HSF1-cancer signature, from low to high. Red bars indicate deaths. Tumors with an average expression value of the signature genes in the top 25^{th} percentile are called “High HSF1-CaSig” (yellow) and the remaining tumors are called “Low HSF1-CaSig” (blue). (B) Log-rank p-values for each of the classifiers indicated was calculated individually across each dataset and results are displayed as a heat map. Corresponding KM curves are provided in Figure S6. (C) Random gene signature analysis of a representative dataset (Pawitan et al., 2005). KM analysis on the dataset to evaluate associations between 10,000 individual randomly generated gene signatures and patient outcome. The random signatures are binned and ordered from least significant to most significant by the KM-generated test statistic. The red arrow indicates the test statistic of the HSF1-CaSig. For reference, black arrows indicate the test statistic of the random signature with the median test statistic (5000th) and the random signature with the 95th percentile test statistic. (D) KM analysis of individuals with ER+/Lymph node negative tumors (Wang et al., 2005) with low HSF1-CaSig (blue) or high HSF1-CaSig (yellow). (E) KM analysis of 947 individuals from the NHS with ER+, lymph-node negative tumors expressing no, low or high nuclear HSF1 as measured by IHC. Data are from the NHS (1976-1997). Log-rank p-values are shown.

FIG. 16. An HSF1-cancer signature is associated with reduced survival in patients with colon or lung cancers. (A) Kaplan-Meier analysis of survival in patients with colon or lung cancer based on low HSF1-CaSig (blue) or high HSF1-CaSig (yellow). Log-rank p-values are shown. (B) Heat map of log-rank p-values for each of the indicated classifiers analyzed individually across four datasets is shown. Corresponding KM curves are provided in FIG. 23.

FIG. 17. BPLER cells are highly dependent on HSF1 for survival and HSF1 activation during malignancy is distinct from its activation by heat-shock. (A) HSF1 (green) and p53 (red) detected by immunofluorescence in HMLER or BPLER xenograft tumors established in mice. Staining for p53 identifies HMLER and BPLER tumor cells. In HMLER cells, HSF1 signal is predominantly seen in p53-low stromal cells. (B) Cells were plated and transduced with either control lentiviral shRNAi constructs (Scramble or GFP) or lentiviral shRNAi constructs that target HSF1 (hA9, ha6). Four days after transduction, the relative viable cell number was measured by a standard dye reduction assay (Alamar blue). (C) Genomic distribution of the regions of HSF1 occupancy (promoter, intragenic or intergenic). (D) Gene set enrichment analysis (GSEA) was performed using the molecular signatures database (MSigDB) web service (http://www.broadinstitute.org/gsea/index.jsp) on genes bound strongly by HSF1 in cancer only (BPLER, only) or bound strongly by HSF1 in both cancer and heat-shocked cells (BPLER and HS). A summary of GSEA results is provided in Tables T2A and T2B. (E) The sequence motif corresponding to the heat-shock element (HSE) is strongly enriched within regions bound strongly by HSF1 in BPLER at 37° C. (BPLER only, top panel) and genes that were well bound in both BPLER cells at 37° C. and in the parental lines (HME and BPE) following heat shock at 42° C., lower panel). The ab initio motif discovery algorithm MEME was used to analyze the 100 bp genomic regions surrounding the HSF1 binding peaks. (F) HSF1 binding of the HSPD1/E1 locus in HMLER, BPLER, HME and BPE cells at 37° C. and HME and BPE cells following heat-shock at 42° C. Arrows indicate the transcription start site of each gene. Reads per million total reads are shown. (G) ChIP was performed from HME, BPE, HMLER or BPLER cells with or without a 1 hr heat-shock at 42° C. using the indicated antibodies (RNA: RNA polymerase II, IGG: pre-immune control). Quantitative PCR was performed on enriched DNA with primers for either the promoter of HSPA6 (top panel), the promoter of DHFR (middle panel) or an intergenic region (bottom pane)) and normalized to input DNA. For clarity, HSPA6 enrichment in the RNA Polymerase IP (top panel) is not shown. (H) mRNA expression analysis showing the effect of heat shock on genes identified as strongly HSF1-bound in BPLER at 37° C. (left) and genes identified as bound strongly in both BPLER cells at 37° C. and parental HME and BPE cells following heat shock (right). The latter group is more heat shock responsive than the former group. The two probes corresponding to HspA6 (HSP70B′) are indicated by an arrow.

FIG. 18. HSF1 depletion by shRNA in HMLER, BPLER and MCF7 cells. Equal amounts of total protein isolated from cells following infection with the indicated lentiviral shRNA constructs were subjected to immunoblotting using an HSF1 antibody (Ab4). ACTB (beta-Actin) was used as a loading control.

FIG. 19. Spectrum of HSF1 binding across select genes in established breast cell lines. (A) ChIP, with indicated antibody, was performed using chromatin from the indicated cell lines. Quantitative PCR was performed on enriched DNA with primers corresponding to the indicated genomic regions and normalized to input DNA. Two biological replicates, each of which contained three technical replicates were performed. Data are shown as mean+/− standard deviation. (B) Scatter plot of HSF1 occupancy at the indicated genes in 12 breast cell lines. Genes are ordered by average level of HSF1 binding, from low (intergenic, top) to high (HspD/E1, bottom). (C) Heat map of the HSF1 binding data depicted in Panel “A”. Low level HSF1 binding is indicated in black and higher levels of HSF1 binding are depicted in yellow. Cell lines are ordered by average level of HSF1 occupancy across all genes, from low (MCF10A) to high (SKBR3). (D) Immunoblot showing HSF1 levels in the cell lines used for the ChIP-Seq experiment presented in FIG. 12. Beta-actin (ACTB) was used as a loading control. (E) HSF1 binding for representative genes (Cks2, Ly6K, Rbm23, CCT6A, and CKS1B) is shown. Arrows indicate transcription start site of each gene. Reads per million total reads are shown.

FIG. 20. Regulation of HSF1-target genes. (A) Quantitative PCR was performed to evaluate expression of selected genes after knockdown of HSF1 using siRNA oligos (48 hrs post-transfection) in 5 cells lines (Breast: BT20, MCF7; Colon: HCT15, HT29; Lung NCIH838). Heat map depicts the average fold-change following transfection with two control siRNA (siGLO RISC-Free siRNA and siGENOME Non-Targeting siRNA #5) and the fold-change induced by HSF1 knockdown with siGenome SMART pool siRNA-Human HSF1. Yellow: positively regulated; Blue: negatively regulated. (B) Western blot of HSF1 (Ab4 antibody) from cell lysates harvested in parallel with samples used to generate mRNA for the quantitative PCR shown in panel A. siCntrl 1: siGLO RISC-Free siRNA; siCntrl 2: siGENOME Non-Targeting siRNA #5. siHSF1: siGenome SMART pool siRNA-Human HSF1. ACTB is the loading control.

FIG. 21. IHC staining of frozen sections of breast and colon tumors used for tumor ChIP-seq analysis in FIG. 14D. The level of nuclear HSF1 signal is reported in FIG. 14D as HSF1 IHC Grade.

FIG. 22. Kaplan-Meier outcome curves for each of the breast cancer datasets evaluated in FIG. 15B. Meta-analysis of 10 publicly available mRNA expression datasets of breast cancer patients. Kaplan-Meier (KM) analysis of patient outcome using the indicated classifiers is shown. For HSF1 activation, tumors with an average expression value of the HSF1-cancer signature in the top 25^{th} percentile were called “High HSF1-CaSig” (red) and the remaining tumors were called “Low HSF1-CaSig” (green). KM curves highlighted in yellow had log-rank p-values<0.05.

FIG. 23: Kaplan-Meier outcome curves for each of the colon and lung cancer datasets evaluated in FIG. 16B. Meta-analysis of four publicly available mRNA expression datasets of colon and lung cancer patients. Kaplan-Meier (KM) analysis of patient outcome using the indicated classifiers is shown. For HSF1 activation, tumors with an average expression value of the HSF1-cancer signature in the top 25^{th} percentile were called “High HSF1-CaSig” (red) and the remaining tumors were called “Low HSF1-CaSig” (green). KM curves highlighted in yellow had log-rank p-values<0.05.

### DETAILED DESCRIPTION OF CERTAIN EMBODIMENTS OF THE INVENTION

### Glossary

For convenience, certain terms employed herein are collected below. It should be understood that any description of a term or concept below or elsewhere herein may be applied wherever such term or concept appears herein.

The term “antibody” refers to an immunoglobulin, whether natural or wholly or partially synthetically produced. An antibody may be a member of any immunoglobulin class, including any of the mammalian, e.g., human, classes: IgG, IgM, IgA, IgD, and IgE, or subclasses thereof, and may be an antibody fragment, in various embodiments of the invention. An antibody can originate from any of a variety of vertebrate (e.g., mammalian or avian) organisms, e.g., mouse, rat, rabbit, hamster, goat, chicken, human, etc. As used herein, the term “antibody fragment” refers to a derivative of an antibody which contains less than a complete antibody. In general, an antibody fragment retains at least a significant portion of the full-length antibody's specific binding ability. Examples of antibody fragments include, but are not limited to, Fab, Fab′, F(ab′)2, scFv, Fv, dsFv diabody, Fd fragments, and domain antibodies. Standard methods of antibody identification and production known in the art can be used to produce an antibody that binds to a polypeptide of interest. In some embodiments, an antibody is a monoclonal antibody. Monoclonal antibodies can be identified and produced, e.g., using hybridoma technology or recombinant nucleic acid technology (e.g., phage or yeast display). In some embodiments, an antibody is a chimeric or humanized or fully human antibody. In some embodiments, an antibody is a polyclonal antibody. In some embodiments an antibody is affinity purified. It will be appreciated that certain antibodies, e.g., recombinantly produced antibodies, can comprise a heterologous sequence not derived from naturally occurring antibodies, such as an epitope tags. In some embodiments an antibody further has a detectable label attached (e.g., covalently attached) thereto (e.g., the label can comprise a radioisotope, fluorescent compound, enzyme, hapten).

“Cancer” is generally used interchangeably with “tumor” herein and encompasses pre-invasive and invasive neoplastic growths comprising abnormally proliferating cells, including malignant solid tumors (carcinomas, sarcomas) and including hematologic malignancies such as leukemias in which there may be no detectable solid tumor mass. As used herein, the term cancer includes, but is not limited to, the following types of cancer: breast cancer; biliary tract cancer; bladder cancer; brain cancer (e.g., glioblastomas, medulloblastomas); cervical cancer; choriocarcinoma; colon cancer; endometrial cancer; esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer such as oral squamous cell carcinoma; ovarian cancer including ovarian cancer arising from epithelial cells, stromal cells, germ cells and mesenchymal cells; pancreatic cancer; prostate cancer, rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullary carcinoma. “Carcinoma” as used herein, refers to a cancer arising or believed to have arisen from epithelial cells, e.g., cells of the cancer possess various molecular, cellular, and/or histological characteristics typical of epithelial cells. “Cancer in situ” (CIS) refers to cancers in which neoplastic cells are present at a location, e.g., as a tumor, but have not detectably invaded beyond the original site where they were discovered, e.g., cancer cells have not detectably passed through the basal lamina. It will be appreciated that a CIS may have undergone some local spread at the time of discovery. In many embodiments a CIS is a tumor that would be classified as Stage 0, e.g., TisN0M0 or TaN0M0 according to the TNM Classification of Malignant Tumours (TNM) (Sobin L H, et al., eds. TNM Classification of Malignant Tumors, 7th ed. Wiley-Blackwell, Oxford 2009). In some embodiments, a CIS is a bladder cancer, breast cancer (e.g., ductal carcinoma in situ of the breast (DCIS)), cervical cancer (in which case the term high grade squamous epithelial lesion (HSIL) may be used instead of CIS), colon cancer, lung cancer (e.g., bronchioloalveolar carcinoma (BAC)), high grade prostatic intraepithelial neoplasia, or skin cancer.

The term “diagnostic method” generally refers to a method that provides information regarding the identity of a disease or condition that affects a subject or whether a subject is suffering from a disease or disorder of interest, such as cancer. For example, a diagnostic method may determine that a subject is suffering from a disease or condition of interest or may identify a disease or condition that affects a subject or may identify a subject suffering from a disease or condition of interest.

“Modulator” refers to an agent or condition that alters, e.g., inhibits (reduces, decreases) or enhances (activates, stimulates, increases), a process, pathway, phenomenon, state, or activity. For example, a modulator of protein activity may increase or decrease the level of one or more activit(ies) of a protein.

The term “prognostic method”, generally refers to a method that provides information regarding the likely course or outcome of a disease regardless of treatment or across treatments (e.g., after adjusting for treatment variables or assuming that a subject receives standard of care treatment). For example, a prognostic method may comprise classifying a subject or sample obtained from a subject into one of multiple categories, wherein the categories correlate with different likelihoods that a subject will experience a particular outcome. For example, categories can be low risk and high risk, wherein subjects in the low risk category have a lower likelihood of experiencing a poor outcome (e.g., within a given time period such as 5 years or 10 years) than do subjects in the high risk category. A poor outcome could be, for example, disease progression, disease recurrence, or death attributable to the disease.

The term “treatment-specific predictive method” generally refers to a method that provides information regarding the likely effect of a specified treatment, e.g., that can be used to predict whether a subject is likely to benefit from the treatment or to predict which subjects in a group will be likely or most likely to benefit from the treatment. It will be understood that a treatment-specific predictive method may be specific to a single treatment or to a class of treatments (e.g., a class of treatments having the same or a similar mechanism of action or that act on the same biological process, pathway or molecular target, etc.). A treatment-specific predictive method may comprise classifying a subject or sample obtained from a subject into one of multiple categories, wherein the categories correlate with different likelihoods that a subject will benefit from a specified treatment. For example, categories can be low likelihood and high likelihood, wherein subjects in the low likelihood category have a lower likelihood of benefiting from the treatment than do subjects in the high likelihood category. In some embodiments, a benefit is increased survival, increased progression-free survival, or decreased likelihood of recurrence. In some embodiments, a “suitable candidate for treatment” with a specified agent refers to a subject for whom there is a reasonable likelihood that the subject would benefit from administration of the agent, e.g., the tumor has one or more characteristics that correlate with a beneficial effect resulting from administration of the agent as compared with, e.g., no treatment or as compared with a standard treatment. In some embodiments, a “suitable candidate for treatment” with an agent refers to a subject for whom there is a reasonable likelihood that the subject would benefit from administration of the agent in combination with (i.e., in addition to) one or more other therapeutic interventions, e.g., the tumor has one or more characteristics that correlate with a beneficial effect from treatment with the agent and the other therapeutic interventions as compared with treatment with the other therapeutic interventions only. In some embodiments, a suitable candidate for treatment with an agent is a subject for whom there is a reasonable likelihood that the subject would benefit from addition of the agent to a standard regimen for treatment of cancer. See, e.g., De Vita, et al., supra for non-limiting discussion of standard regimens for treatment of cancer.

“Expression” refers to the cellular processes involved in producing RNA and protein such as, but not limited to, transcription, RNA processing, and translation.

As used herein, the term “gene product” (also referred to as a “gene expression product”) encompasses products resulting from expression of a gene, such as RNA transcribed from a gene and polypeptides arising from translation of mRNA. RNA transcribed from a gene can be non-coding RNA or coding RNA (e.g., mRNA). It will be appreciated that gene products may undergo processing or modification by a cell. For example, RNA transcripts may be spliced, polyadenylated, etc., prior to mRNA translation, and/or polypeptides may undergo co-translational or post-translational processing such as removal of secretion signal sequences or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Exemplary databases include, e.g., GenBank, RefSeq, Gene, UniProtKB/SwissProt, UniProtKB/Trembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species due to natural allelic variation. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations frequently do not alter the encoded amino acid sequence, although DNA polymorphisms that lead to changes in the amino acid sequences of the encoded proteins can exist. It will also be understood that multiple isoforms of certain proteins encoded by the same gene may exist as a result of alternative RNA splicing or editing. Examples of polymorphic variants can be found in, e.g., the Single Nucleotide Polymorphism Database (dbSNP) (available at the NCBI website at www.ncbi.nlm.nih.gov/projects/SNP/), which contains single nucleotide polymorphisms (SNPs) as well as other types of variations (see, e.g., Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (Md.): National Center for Biotechnology Information (US); 2002 (www.ncbi.nlm.nih.gov/bookshelf/br.fcgi?book=handbook&part=ch5). In general, where aspects of the invention relate to a gene or gene product it should be understood that embodiments relating to such isoforms or allelic variants are encompassed unless indicated otherwise. For example, in general, allelic variants and most isoforms would be detectable using the same reagents (e.g., antibodies, probes, etc.) and methods. Certain embodiments may be directed to a particular sequence or sequences, e.g., a particular allele or isoform. One of ordinary skill in the art could readily develop reagents and methods that could distinguish between different isoforms or allelic variants or could verify that particular isoform(s) or allelic variant(s) are detected by a particular detection method or reagent.

“Isolated”, in general, means 1) separated from at least some of the components with which it is usually associated in nature; 2) prepared or purified by a process that involves the hand of man; and/or 3) not occurring in nature, e.g., present in an artificial environment.

“Nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments a nucleic acid comprises standard nucleosides (abbreviated A, G, C, T, U). In other embodiments a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos, in various embodiments. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, antisense, primer, or probe molecules may be used in various embodiments of the invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., relative to RNA or DNA of the same length and strandedness. A nucleic acid may comprise a detectable label, e.g., a fluorescent dye, radioactive atom, etc. “Oligonucleotide” refers to a relatively short nucleic acid, e.g., typically between about 4 and about 100 nucleotides long. Where reference is made herein to a polynucleotide, it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence, if presented herein, is presented in a 5′ to 3′ direction unless otherwise indicated.

“Polypeptide” refers to a polymer of amino acids. The terms “protein” and “polypeptide” are used interchangeably herein. A peptide is a relatively short polypeptide, typically between about 2 and 100 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity thereto. Exemplary modifications include phosphorylation, glycosylation, SUMOylation, acetylation, methylation, acylation, etc. In some embodiments, a polypeptide is modified by attachment of a linker useful for conjugating the polypeptide to or with another entity. Polypeptides may be present in or purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence, if presented herein, is presented in an N-terminal to C-terminal direction unless otherwise indicated.

A “sample” as used herein can be any biological specimen that contains cells, tissue, or cellular material (e.g., cell lysate or fraction thereof). Typically, a sample is obtained from (i.e., originates from, was initially removed from) a subject. Methods of obtaining such samples are known in the art and include, e.g., tissue biopsy such as excisional biopsy, incisional biopsy, or core biopsy; fine needle aspiration biopsy; brushings; lavage; or collecting body fluids such as blood, sputum, lymph, mucus, saliva, urine, etc., etc. In many embodiments, a sample contains at least some intact cells at the time it is removed from a subject and, in many embodiments, the sample retains at least some of the tissue microarchitecture. In many embodiments a sample will have been obtained from a tumor either prior to or after removal of the tumor from a subject. A sample may be subjected to one or more processing steps after having been obtained from a subject and/or may be split into one or more portions, which may entail removing or discarding part of the original sample. It will be understood that the term “sample” encompasses such processed samples, portions of samples, etc., and such samples are still considered to have been obtained from the subject from whom the initial sample was removed. In many embodiments, a sample is obtained from an individual who has been diagnosed with cancer or is at increased risk of cancer, is suspected of having cancer, or is at risk of cancer recurrence. A sample used in a method of the present invention may have been procured directly from a subject, or indirectly by receiving the sample from one or more persons who procured the sample directly from the subject, e.g., by performing a biopsy or other procedure on the subject. A “tumor sample” is a sample that includes at least some cells, tissue, or cellular material obtained from a tumor. In general, a “sample” as used herein is typically a tumor sample or a sample obtained from tissue being evaluated for presence of a tumor.

The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments a small molecule is an artificial (non-naturally occurring) molecule. In some embodiments, a small molecule is non-polymeric. In some embodiments, a small molecule is not an amino acid. In some embodiments, a small molecule is not a nucleotide. In some embodiments, a small molecule is not a saccharide. In some embodiments, the term “small molecule” excludes molecules that are ingredients found in standard tissue culture medium.

“Specific binding” generally refers to a physical association between a target molecule or complex (e.g., a polypeptide) and a binding agent such as an antibody or ligand. The association is typically dependent upon the presence of a particular structural feature of the target such as an antigenic determinant, epitope, binding pocket or cleft, recognized by the binding agent. For example, if an antibody is specific for epitope A, the presence of a polypeptide containing epitope A or the presence of free unlabeled A in a reaction containing both free labeled A and the binding molecule that binds thereto, will typically reduce the amount of labeled A that binds to the binding molecule. It is to be understood that specificity need not be absolute but generally refers to the context in which the binding occurs. For example, it is well known in the art that antibodies may in some instances cross-react with other epitopes in addition to those present in the target. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule such as HSF1). It is also to be understood that specificity may be evaluated in the context of additional factors such as the affinity of the binding agent for the target versus the affinity of the binding agent for other targets, e.g., competitors. If a binding agent exhibits a high affinity for a target molecule that it is desired to detect and low affinity for nontarget molecules, the antibody will likely be an acceptable reagent. Once the specificity of a binding molecule is established in one or more contexts, it may be employed in other contexts, e.g., similar contexts such as similar assays or assay conditions, without necessarily re-evaluating its specificity. In some embodiments specificity of an antibody can be tested by performing an appropriate assay on a sample expected to lack the target (e.g., a sample from cells in which the gene encoding the target has been disabled or effectively inhibited) and showing that the assay does not result in a signal significantly different to background.

“Subject” refers to any individual who has or may have cancer or is at risk of developing cancer or cancer recurrence. The subject is preferably a human or non-human animal, including but not limited to animals such as rodents (e.g., mice, rats, rabbits), cows, pigs, horses, chickens, cats, dogs, primates, etc., and is typically a mammal, and in many embodiments is a human. In some embodiments a subject is female. In some embodiments a subject is male. A subject may be referred to as a “patient”.

“Vector” is used herein to refer to a nucleic acid or a virus or portion thereof (e.g., a viral capsid or genome) capable of mediating entry of, e.g., transferring, transporting, etc., a nucleic acid molecule into a cell. Where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not taken up (e.g., been transfected with) or maintain the vector. Markers include, for example, proteins that increase or decrease either resistance or sensitivity to antibiotics (e.g., an antibiotic-resistance gene encoding a protein that confers resistance to an antibiotic such as puromycin, G418, hygromycin or blasticidin) or other compounds, enzymes whose activities are detectable by assays known in the art (e.g., β-galactosidase or alkaline phosphatase), and proteins or RNAs that detectably affect the phenotype of transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylation signals and/or a 3′ untranslated regions. Vectors often include one or more appropriately positioned sites for restriction enzymes, to facilitate introduction into the vector of the nucleic acid to be expressed. An expression vector typically comprises sufficient cis-acting elements for expression; other elements required or helpful for expression can be supplied by the cell or in vitro expression system into which the vector is introduced.

Various techniques known in the art may be employed for introducing nucleic acid molecules into cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). For purposes of convenience the term “transfection” may be used to refer to any and all such techniques. Markers can be used for the identification and/or selection of cells that have taken up the vector and, typically, express the nucleic acid. Cells can be cultured in appropriate media to select such cells and, optionally, establish a stable cell line, e.g., polyclonal or monoclonal cell line. For example, a stable cell line can be composed of cells that have an exogenous nucleic acid encoding a gene product to be expressed integrated into the genome of the cells or, in some embodiments, present on an episome that is maintained and transmitted with high fidelity to daughter cells during cell division. Methods of generating stable cell lines are well known in the art and include, e.g., transfection, viral infection (e.g., using retroviruses (e.g., lentiviruses), adenoviruses, adeno-associated viruses, herpesviruses, etc.), typically followed by selection of cells that have taken up and stably maintain an introduced nucleic acid or portion thereof. A stable cell line may be polyclonal (descended from a pool of cells that have taken up a vector) or may be monoclonal (descended from a single cell that has taken up a vector).

Selection of appropriate expression control elements may be based at least in part on the cell type and species in which the nucleic acid is to be expressed and/or the purposes for which the vector is to be used. One of ordinary skill in the art can readily select appropriate expression control elements and/or expression vectors. In some embodiments, expression control element(s) are regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, phosphoglycerate kinase, etc. Regulatable (e.g., inducible or repressible) expression systems such as the Tet-On and Tet-Off systems (regulatable by tetracycline and analogs such as doxycycline) and others that can be regulated by small molecules such as hormone receptor ligands (e.g., steroid receptor ligands, which may or may not be steroids), metal-regulated systems (e.g., metallothionein promoter), etc.

HSF1 as a Marker for Cancer Classification

Heat shock factor 1 (HSF1), also known as heat shock transcription factor 1, is a multifaceted transcription factor that governs the cellular response to a variety of disruptions in protein homeostasis, serving as the master transcriptional regulator of the cellular response to heat and various other stressors in mammals. Under normal (non-stressed) conditions, HSF1 is predominantly located in the cytoplasm as a monomer, which is unable to bind DNA. Upon exposure to stressors, HSF1 is activated and translocates to the nucleus, where it regulates gene expression by binding to DNA sequence motifs known as heat-shock elements (HSE) located in the promoter regions of target genes. To protect the proteome under various physiologic or environmental stresses, HSF1 drives the production of classic heat-shock proteins (HSPs) such as HSP27, HSP70 and HSP90 that act as protein chaperones. Among other activities, HSPs facilitate proper protein folding and assembly and help prevent deleterious protein aggregation. This response, termed the heat shock response (HSR), is present in eukaryotes ranging from yeast to humans (1-3).

As described herein, Applicants have discovered that HSF1 expression and activation are increased across a broad range of human tumor types and that increased HSF1 expression and activation in tumors are an indicator of aggressive tumor phenotypes and poor clinical outcome. For example, Applicants observed a striking increase in the levels of HSF1, as well as a shift in its localization from the cytoplasm to the nucleus, in a panel of human breast cancer samples as compared with normal breast tissue. Applicants also found that HSF1 expression and nuclear localization were increased in lung, colon, prostate, cervical carcinomas as well in other tumors including malignant peripheral nerve sheath tumor. Nuclear HSF1 levels were elevated in ˜80% of in situ and invasive breast carcinomas analyzed. In invasive carcinomas, HSF1 expression was associated with high histologic grade, larger tumor size, and nodal involvement at diagnosis. Applicants hypothesized that this increase in nuclear HSF1 might be associated with poor prognosis. To investigate this possibility, Applicants examined the relationship between HSF1, clinicopathological characteristics, and survival outcomes among over 1,800 invasive breast cancer cases from the Nurses' Health Study. They found that increased levels of HSF1 expression and nuclear localization in tumor samples correlated with high histologic grade, larger tumor size, and nodal involvement at diagnosis in invasive breast carcinomas. Increased HSF1 levels and nuclear localization of HSF1 were associated with advanced clinical stage at the time of diagnosis and with increased mortality. The prognostic value of HSF1 protein was retained after adjusting for age, stage, grade, and adjuvant therapy. Thus, HSF1 is an independent prognostic indicator of outcome in breast cancer. Increased HSF1 expression and activation were shown to correlate with decreased overall survival and decreased disease free progression in a group of 70 stage 1 lung cancer patients and with decreased survival in colon cancer patients. Thus, increased HSF1 expression and activation in tumors correlates with aggressive tumor phenotype and worse clinical outcomes.

Without wishing to be bound by any theory, Applicants hypothesized that HSF1 may in part enable more aggressive cancer phenotypes and lead to worse clinical outcomes as a result of HSP elevation, driven by HSF1 responding to the protein folding conditions that are common in malignancies, such as increased protein load from dysregulation of the translation machinery, accumulation of mutated or fusion proteins, and imbalances in the stoichiometry of protein complexes due to aneuploidy. However, Applicants hypothesized that HSF1's role in cancer is much broader. Malignant transformation alters cellular physiology and imposes significant metabolic and genetic stresses in addition to proteomic stresses. HSF1's impact on cell cycle control, survival signaling, and energy metabolism during tumor initiation and progression may allow tumor cells to cope with these malignancy-associated stressors and/or may facilitate progression to invasive cancer and/or emergence of drug resistance by enabling the generation of greater phenotypic diversity. Furthermore, as described herein, Applicants found that HSF1 has a direct and pervasive role in cancer biology. Extending far beyond protein folding and stress, HSF1-bound genes are involved in many facets of tumorigenesis, tumor growth, persistence, progression, and/or response to therapy, including the cell cycle, apoptosis, energy metabolism, and other processes.

In some aspects, the invention provides methods of classifying a sample with respect to cancer diagnosis (e.g., the presence or absence of cancer), cancer aggressiveness, cancer outcome, or cancer treatment selection, based at least in part on assessing the level of HSF1 expression or HSF1 activation in the sample. In some aspects, the invention provides methods of cancer diagnosis, prognosis, or treatment-specific prediction, based at least in part on assessing the level of HSF1 expression or HSF1 activation in a sample, e.g., a tumor sample or suspected tumor sample. In some embodiments, the cancer is an adenocarcinoma. In some embodiments the cancer is a breast, lung, colon, prostate, or cervical cancer, e.g., a breast, lung, colon, prostate, or cervical adenocarcinoma. In some embodiments the tumor is a squamous cell carcinoma. In some embodiments the tumor is not a squamous cell carcinoma. In some embodiments the cancer is a sarcoma. In some embodiments the sarcoma is a nerve sheath tumor, e.g., a peripheral nerve sheath tumor. In some embodiments the nerve sheath tumor is a malignant nerve sheath tumor, e.g., a malignant peripheral nerve sheath tumor. In some embodiments a tumor is a Stage I tumor as defined in the TNM Classification of Malignant Tumours (2009). In some embodiments a tumor is a Stage II tumor as defined in the TNM Classification of Malignant Tumours (2009). It will be understood that results of an assay of HSF1 expression or HSF1 activation may be used in combination with results from other assays, or other information, to provide a sample classification, diagnosis, prognosis, or prediction relating to cancer, cancer outcome, or treatment response. Such combination methods are within the scope of the invention.

In some aspects, the invention relates to methods for classifying a sample according to the level of HSF1 expression (i.e., the level of expression of the HSF1 gene) or according to the level of HSF1 activation in the sample. For purposes hereof, a method that comprises assessing HSF1 expression or assessing HSF1 activation may be referred to as an “HSF1-based method”. A procedure that is used to assess (detect, measure, determine, quantify) HSF1 expression or HSF1 activation may be referred to as an “HSF1-based assay”. It will be understood that either HSF1 expression, HSF1 activation, or both, can be assessed in various embodiments of the invention. Certain assays such as IHC can be used to assess both expression and activation. In general, as described further in the Examples, the level of HSF1 activation detected in tumor samples correlated with the level of HSF1 expression, e.g., samples that exhibited increased nuclear HSF1 levels tended to have increased HSF1 protein expression.

In some embodiments, the level of HSF1 expression is assessed by determining the level of an HSF1 gene product in the sample. Thus in some embodiments, the invention relates to methods for classifying a sample according to the level of an HSF1 gene product in the sample. In some embodiments, the invention provides a method of classifying a sample, the method comprising steps of: (a) providing a sample obtained from a subject; and (b) assessing HSF1 expression in the sample, wherein the level of HSF1 expression is correlated with a phenotypic characteristic, thereby classifying the sample with respect to the phenotypic characteristic. In some embodiments, the invention provides a method of classifying a sample, the method comprising steps of: (a) providing a sample obtained from a subject; and (b) determining the level of an HSF1 gene product in the sample, wherein the level of an HSF1 gene product is correlated with a phenotypic characteristic, thereby classifying the sample with respect to the phenotypic characteristic. In some embodiments the phenotypic characteristic is presence or absence of cancer. In some embodiments, the cancer is invasive cancer. In some embodiments the sample does not show evidence of invasive cancer, and the phenotypic characteristic is presence or absence of pre-invasive cancer (cancer in situ). In some embodiments the phenotypic characteristic is cancer prognosis. In some embodiments the phenotypic characteristic is predicted treatment outcome. In some embodiments the HSF1 gene product is HSF1 mRNA. In some embodiments the HSF1 gene product is HSF1 polypeptide.

In some aspects, the invention provides a method of classifying a sample, the method comprising: (a) determining the level of HSF1 expression or the level of HSF1 activation in a sample; (b) comparing the level of HSF1 expression or HSF1 activation with a control level of HSF1 gene expression or HSF1 activation; and (c) classifying the sample with respect to cancer diagnosis, wherein a greater (increased) level of HSF1 gene expression or HSF1 activation in the sample as compared with the control level of HSF1 expression or HSF activation, respectively, is indicative of the presence of cancer. In some embodiments, a greater level of HSF1 expression or HSF1 activation in the sample is indicative of the presence of in situ cancer in a sample that does not show evidence of invasive cancer. If the level of HSF1 expression or HSF1 activation is not increased (e.g., HSF1 is not detectable or is not significantly greater than present in normal tissue), then cancer is not diagnosed based on HSF1.

In some aspects, the invention provides a method of classifying a sample, the method comprising: (a) determining the level of HSF1 expression or the level of HSF1 activation in a sample obtained from a tumor; (b) comparing the level of HSF1 expression or HSF1 activation with a control level of HSF1 gene expression or HSF1 activation; and (c) classifying the sample with respect to cancer prognosis, wherein a greater level of HSF1 gene expression or HSF activation in the sample obtained from the tumor as compared with the control level of HSF1 gene expression or HSF activation, respectively, is indicative that the sample originated from a tumor that belongs to a poor prognosis class. In some aspects, the invention provides a method of classifying a tumor, the method comprising: (a) determining the level of HSF1 expression or the level of HSF1 activation in a sample obtained from a tumor; (b) comparing the level of HSF1 expression or HSF1 activation with a control level of HSF1 gene expression or HSF1 activation; and (c) classifying the sample with respect to cancer prognosis, wherein a greater level of HSF1 gene expression or HSF activation in the sample obtained from the tumor as compared with the control level of HSF1 gene expression or HSF1 activation, respectively, is indicative that the tumor belongs to a poor prognosis class.

In some aspects, the invention relates to methods for classifying a sample according to the level of HSF1 activation in cells of the sample. As used herein, “HSF1 activation” refers the process in which HSF1 polypeptide is phosphorylated, trimerizes, and translocates to the nucleus, where it binds to DNA sequences and regulates expression of genes containing such sequences (e.g., in their promoter regions) (“HSF1-regulated genes”). In some embodiments, the invention is directed to a method of classifying a sample with respect to a phenotypic characteristic, the method comprising steps of: (a) providing a sample obtained from a subject; and (b) determining the level of activation of HSF1 polypeptide in the sample, wherein the level of activation of an HSF1 polypeptide is correlated with a phenotypic characteristic, thereby classifying the sample with respect to the phenotypic characteristic. In some embodiments the sample does not show evidence of invasive cancer, and the phenotypic characteristic is presence or absence of pre-invasive cancer. In some embodiments the phenotypic characteristic is cancer prognosis. In some embodiments the phenotypic characteristic is predicted treatment outcome. In some embodiments, the level of HSF1 activation is assessed by determining the level of nuclear HSF1 in the sample. Thus in some embodiments the invention relates to methods for classifying a sample according to the level of nuclear HSF1 in the sample. In some embodiments, assessing the level of HSF1 activation comprises assessing HSF1 activity. In some embodiments, assessing the level of HSF1 activity comprises measuring expression of one or more HSF1-regulated genes. In some embodiments assessing the level of HSF1 activity comprises measuring expression of one or more HSF1 cancer program (HSF1-CP) genes. In some embodiments assessing the level of HSF1 activity comprises measuring expression of one or more HSF1-cancer signature set (HSF1-CSS), Group A, Group B, HSF1-CaSig2, HSF1-CaSig3, refined HSF1-CSS, Module 1, Module 2, Module 3, Module 4, or Module 5 genes. HSF1-CP genes, HSF1-CSS genes, Group A, Group B, HSF1-CaSig2, HSF1-CaSig3, refined HSF1-CSS, Module 1, Module 2, Module 3, Module 4, and Module 5 genes are described in further detail elsewhere herein. In some embodiments, assessing the level of HSF1 activity comprises measuring binding of HSF1 to the promoter region of one or more HSF1-regulated genes. In some embodiments assessing the level of HSF1 activity comprises measuring binding of HSF1 to a regulatory region, e.g., a promoter region or a distal regulatory region of one or more HSF1-CP genes, e.g., one or more HSF1-CSS, Group A, Group B, HSF1-CaSig2, HSF1-CaSig3, refined HSF1-CSS, Module 1, Module 2, Module 3, Module 4, or Module 5 genes. In some embodiments “one or more” genes is at least 1, 2, 3, 4, 5, 10, 15, 20, 25, 30, 40, 50, 60, 70, 80, 90, 100, 150, 200, 250, 300, 350, 400, or 450, up to the total number of genes in a set or list of genes. In some embodiments “one or more” genes is at least 5%, 10%, 20%, 30%, 40%, 50%, 60%, 70%, 80%, 90%, 95%, 96%, 97%, 98%, 99%, or more, up to 100% in a set or list of genes.

In some aspects of the invention, detection of increased HSF expression or activation in a sample is of use for diagnosis of cancer, e.g., for detection of cancer. According to certain of the methods of the invention, samples can be classified as belonging to (i.e., obtained from) an individual who has cancer or is likely to develop cancer. Among other things, the present invention provides the recognition that HSF1 expression in many instances initially becomes elevated during the in situ stage of malignant transformation, prior to invasion. In some aspects of the invention, detection of elevated (increased) HSF expression or activation in a sample is of use for early diagnosis of cancer, e.g., for detection of cancer in situ. According to certain of the methods of the invention, samples can be classified as belonging to (i.e., obtained from) an individual who has cancer in situ (CIS) or is likely to develop CIS or who has CIS and is likely to develop invasive cancer. In some embodiments the sample can be classified as belonging to (i.e., obtained from) an individual who has or is likely to develop ductal carcinoma in situ of the breast (DCIS).

In some embodiments, detection of increased HSF1 expression or activation in a sample indicates that a subject has an increased likelihood of having CIS or developing CIS than would be the case in the absence of increased HSF1 expression or activation. In some embodiments, detection of increased HSF1 expression or activation in a sample is of use to detect a CIS before it becomes detectable on physical examination or, in some embodiments, before it becomes detectable on imaging. In some embodiments, detection of increased HSF1 expression or activation in a sample may be used to help differentiate lesions that are malignant or that have significant potential to become invasive or metastasize from benign lesions. In accordance with certain embodiments of the invention, a lesion has an increased likelihood of being malignant or having significant potential to become invasive or metastasize if increased HSF1 expression or activation is detected in the sample than would be the case if increased HSF1 expression or activation is not detected. Detection of increased HSF1 expression or activation in a sample could, for example, indicate a need for additional or more frequent follow-up of the subject or for treatment of the subject from whom the sample was obtained. In some embodiments, detection of elevated HSF1 expression or activation in a sample is used together with one or more other indicators of dysplasia and/or neoplasia to detect the presence of CIS or to differentiate lesions that are malignant or that have significant potential to become invasive or metastasize from benign lesions. In some embodiments, detection of elevated HSF1 expression may enable classification of a sample that could not be reliably classified (e.g., as high risk or low risk) using standard histopathologic criteria. It will be understood that whether a sample (or tumor from which the sample originated) has an increased level of HSF1 expression or HSF1 activation can be determined by comparing the sample with a suitable control.

In some aspects, the invention provides method of identifying CIS, comprising assessing expression of HSF1 or activation of HSF1 in a tissue or cell sample, wherein the sample does not show evidence of invasive cancer, and wherein increased expression of HSF1 or increased activation of HSF1 in the sample is indicative of CIS. In some aspects, the invention provides a method of predicting the likelihood that a subject will develop invasive cancer, comprising assessing expression of the HSF1 gene or activation of HSF1 in a tissue or cell sample obtained from the subject, wherein increased expression of HSF1 or increased activation of HSF1 in the sample is indicative of an increased likelihood that the subject will develop invasive cancer. In some aspects, the invention provides a method of method of diagnosing CIS in a subject, comprising assessing expression of HSF1 or activation of HSF1 in a tissue or cell sample obtained from the subject, wherein the sample does not show evidence of invasive cancer, and wherein increased expression of HSF1 or increased activation of HSF1 in the sample indicates the presence of CIS in the subject.

In some embodiments, classification of DCIS lesions based on HSF1 expression or HSF1 activation may be used to differentiate DCIS lesions that are likely to progress to invasive cancer from those lesions that are likely to remain unchanged over extended periods of time or to disappear. DCIS lesions that exhibit elevated HSF1 expression or activation in a sample obtained from the lesion would be classified as having a greater likelihood of progression (e.g., within a time period such as 1 year) than lesions that do not exhibit elevated HSF1 expression or HSF1 activation in a sample obtained therefrom.

In some embodiments, a method of identifying, detecting, or diagnosing cancer, e.g., cancer in situ, is applied to a sample obtained from a subject who is at increased risk of cancer (e.g., increased risk of developing cancer or having cancer) or is suspected of having cancer or is at risk of cancer recurrence. A subject at increased risk of cancer may be, e.g., a subject who has not been diagnosed with cancer but has an increased risk of developing cancer as compared with a control, who may be matched with regard to one or more demographic characteristics such as age, gender, etc. For example, the subject may have a risk at least 1.2, 1.5, 2, 3, 5, 10 or more times that of an age-matched control (e.g., of the same gender), in various embodiments of the invention. It will be understood that “age-matched” can refer to the same number of years of age as the subject or within the same age range as the subject (e.g., a range of 5 or 10 years). For example, a control may be up to 5 years older or younger than the subject. Determining whether a subject is considered “at increased risk” of cancer is within the skill of the ordinarily skilled medical practitioner. Any suitable test(s) and/or criteria can be used. For example, a subject may be considered “at increased risk” of developing cancer if any one or more of the following apply: (i) the subject has a mutation or genetic polymorphism that is associated with increased risk of developing or having cancer relative to other members of the general population not having such mutation or genetic polymorphism (e.g., certain mutations in the BRCA1 or BRCA2 genes are well known to be associated with increased risk of a variety of cancers, including breast cancer and ovarian cancer, mutations in tumor suppressor genes such as Rb or p53 can be associated with a variety of different cancer types); (ii) the subject has a gene or protein expression profile, and/or presence of particular substance(s) in a sample obtained from the subject (e.g., blood), that is/are associated with increased risk of developing or having cancer relative to other members of the general population not having such gene or protein expression profile, and/or substance(s) in a sample obtained from the subject; (iii) the subject has one or more risk factors such as having a family history of cancer, having been exposed to a tumor-promoting agent or carcinogen (e.g., a physical carcinogen, such as ultraviolet or ionizing radiation; a chemical carcinogen such as asbestos, tobacco components or other sources of smoke, aflatoxin, or arsenic; a biological carcinogen such as certain viruses or parasites), or has certain conditions such as chronic infection/inflammation that are correlated with increased risk of cancer; (iv) the subject is over a specified age, e.g., over 60 years of age, etc. In the case of breast cancer, a subject diagnosed as having lobular carcinoma in situ (LCIS) is at increased risk of developing cancer. A subject suspected of having cancer may be a subject who has one or more symptoms of cancer or who has had a diagnostic procedure performed that suggested or was at least consistent with the possible existence of cancer but was not definitive. A subject at risk of cancer recurrence can be any subject who has been treated for cancer such that the cancer was rendered undetectable as assessed, for example, by appropriate methods for cancer detection.

According to certain methods of the invention, a sample, tumor, or subject can be classified as belonging to a particular class of outcome based at least in part on the level of HSF1 expression or HSF1 activation. For example, in some embodiments, a sample, tumor, or subject can be classified as belonging to a high risk class (e.g., a class with a prognosis for a high likelihood of recurrence after treatment or a class with a prognosis for a high likelihood of discovery of metastasis post-diagnosis or a class with a poor prognosis for survival after treatment) or a low risk class (e.g., a class with a prognosis for a low likelihood of recurrence after treatment or a class with a prognosis for a low likelihood of discovery of metastasis post-diagnosis or a class with a good prognosis for survival after treatment). In some embodiments, survival after treatment is assessed 5 or 10 years after diagnosis, wherein increased expression of HSF1 or increased activation of HSF1 is predictive of decreased likelihood of survival at 5 years or 10 years post-diagnosis. In some embodiments, increased expression of HSF1 or increased activation of HSF1 is predictive of decreased mean (average) or median survival. In some embodiments survival is overall survival, wherein increased expression of HSF1 or increased activation of HSF1 is predictive of decreased overall survival (increased overall mortality). In some embodiments survival is disease-specific survival, wherein increased expression of HSF1 or increased activation of HSF1 is predictive of decreased disease-specific survival (i.e., increased disease-specific mortality), wherein “disease-specific” in the context of outcome, refers to considering only deaths due to cancer, e.g., breast cancer.

According to certain methods of the invention, a sample, tumor, or subject can be classified as belonging to a particular class with regard to tumor aggressiveness. For example, a sample or tumor can be classified into a more aggressive class or a less aggressive class or a subject can be classified as having a tumor that is more aggressive or less aggressive. “More aggressive” in this context means that the sample or tumor has one or more features that correlate with a poor outcome. A poor outcome may be, e.g., progression (e.g., after treatment), recurrence after treatment, or cancer-related mortality (e.g., within 5, 10, or 20 years after treatment). For example, a tumor classified as more aggressive may have an increased likelihood of having metastasized locally or to remote site(s) at the time of diagnosis, an increased likelihood of metastasizing or progressing locally (e.g., within a specified time period after diagnosis such as 1 year, 2 years, etc.), an increased likelihood of treatment resistance (e.g., a decreased likelihood of being eradicated or rendered undetectable by treatment). In some aspects, the invention provides a method of assessing the aggressiveness of a tumor, the method comprising: determining the level of HSF1 expression or the level of HSF1 activation in a sample obtained from the tumor, wherein if the level of HSF1 gene expression or HSF activation in the sample obtained from the tumor is increased, the tumor is classified as belonging to a more aggressive class. In some aspects, the invention provides a method of assessing the aggressiveness of a tumor, the method comprising: (a) determining the level of HSF1 expression or the level of HSF1 activation in a sample obtained from the tumor; (b) comparing the level of HSF1 expression or HSF1 activation with a control level of HSF1 gene expression or HSF1 activation; and (c) assessing the aggressiveness of the tumor based at least in part on the result of step (b), wherein a greater level of HSF1 gene expression or HSF activation in the sample obtained from the tumor as compared with the control level of HSF1 gene expression or HSF activation, respectively, is indicative of increased aggressiveness.

In some aspects, the invention provides a method of assessing the likelihood that a tumor has metastasized, the method comprising: determining the level of Heat Shock Factor-1 (HSF1) expression or the level of HSF1 activation in a sample obtained from the tumor, wherein if the level of HSF1 gene expression or HSF activation in the sample obtained from the tumor is increased, the tumor has an increased likelihood of having metastasized. In some aspects, the invention provides a method of assessing the likelihood that a tumor will metastasize, the method comprising: determining the level of HSF1 expression or the level of HSF1 activation in a sample obtained from the tumor, wherein if the level of HSF1 gene expression or HSF activation in the sample obtained from the tumor is increased, the tumor has an increased likelihood of metastasizing. In some aspects, the invention provides a method of assessing the likelihood that a tumor has metastasized, the method comprising: (a) determining the level of HSF1 expression or the level of HSF1 activation in a sample obtained from the tumor; (b) comparing the level of HSF1 expression or HSF1 activation with a control level of HSF1 gene expression or HSF1 activation, wherein a greater level of HSF1 gene expression or HSF activation in the sample obtained from the tumor as compared with a control level is indicative of a greater likelihood that the tumor has metastasized. In some aspects, the invention provides a method of assessing likelihood that a tumor will metastasized, the method comprising: (a) determining the level of HSF1 expression or the level of HSF1 activation in a sample obtained from the tumor; (b) comparing the level of HSF1 expression or HSF1 activation with a control level of HSF1 gene expression or HSF1 activation, wherein a greater level of HSF1 gene expression or HSF activation in the sample obtained from the tumor as compared with a control level is indicative of a greater likelihood that the tumor will metastasize.

An HSF1-based method of the invention may be useful for selecting a treatment regimen for a subject. For example, such results may be useful in determining whether a subject should receive, e.g., would likely benefit from, administration of one or more chemotherapeutic agents (chemotherapy), hormonal therapy, an anti-HER2 agent, or other treatment such as radiation. In some embodiments, “chemotherapeutic agent” refers to an anti-tumor agent that has cytotoxic or cytostatic properties and does not act primarily by interacting with (e.g., interfering with) a hormonal pathway that is specific or relatively specific to particular cell type(s). Exemplary chemotherapeutic agents include anti-metabolites, alkylating agents, microtubule stabilizers or microtubule assembly inhibitors (e.g., taxanes or vinca alkaloids), topoisomerase inhibitors, and DNA intercalators (e.g., anthracycline antibiotics). Such agents are frequently administered systemically. Often, multiple agents are administered. Exemplary treatment regimens for breast cancer include CMF (cyclophosphamide, methotrexate, and 5-FU), AC (doxorubicin and cyclophosphamide), and anthracycline-based regimens. Capecitabine is is a prodrug, that is enzymatically converted to 5-fluorouracil following administration (e.g., in tumor tissue) and is a component of a number of breast cancer treatment regimens. Tegafur is another 5-FU prodrug, which may be administered together with uracil, a competitive inhibitor of dihydropyrimidine dehydrogenase. A “hormonal therapy” (also termed “endocrine therapy”) refers to an antitumor agent that acts primarily by interacting with the endocrine system, e.g., by interfering with a hormonal pathway that is active in a hormonally responsive tissue such as breast, prostate, or endometrium. Exemplary hormonal therapies include, e.g., drugs that inhibit the production or activity of hormones that would otherwise contribute to tumor cell survival, proliferation, etc. For example, in the case of breast cancer, hormonal therapy can comprise an agent that inhibits ER signaling. The agent may interact with and inhibit the ER or inhibit estrogen biosynthesis. In some embodiments hormonal therapy comprises a selective estrogen receptor modulator (SERM) such as tamoxifen, raloxifene, or toremifene. It will be appreciated that SERMs can act as ER inhibitors (antagonists) in breast tissue but, depending on the agent, may act as activators (e.g., partial agonists) of the ER in certain other tissues (e.g., bone). It will also be understood that tamoxifen itself is a prodrug that has relatively little affinity for the ER but is metabolized into active metabolites such as 4-hydroxytamoxifen (afimoxifene) and N-desmethyl-4-hydroxytamoxifen (endoxifen). Such active metabolites may be used as ER inhibitors. In some embodiments, hormonal therapy comprises a selective estrogen receptor down-regulators (SERD) such as fulvestrant or CH4986399. In some embodiments hormonal therapy comprises an agent that inhibits estrogen biosynthesis. For example, estrogen deprivation can be achieved using inhibitors that block the last stage in the estrogen biosynthetic sequence, i.e., the conversion of androgens to estrogens by the enzyme aromatase (“aromatase inhibitors”). Aromatase inhibitors include, e.g., letrozole, anastrazole, and exemestane. In the case of prostate cancer, “hormonal therapy” can comprise administering an agent that interferes with androgen receptor (AR) signaling. For example, antiandrogens are drugs that bind to and inhibit the AR, blocking the growth- and survival-promoting effects of testosterone on certain prostate cancers. Examples include flutamide and bicalutamide. Analogs of gonadotropin-releasing hormone (GnRH) can be used to suppress production of estrogen and progesterone from the ovaries, or to suppress testosterone production from the testes. Leuprolide and goserelin are GnRH analogs which are used primarily for the treatment of hormone-responsive prostate cancer.

“Adjuvant therapy” refers to administration of one or more antitumor agents in connection with, e.g., following, local therapy such as surgery and/or radiation. Adjuvant therapy may be used, e.g., when a cancer appears to be largely or completely eradicated, but there is risk of recurrence. Such therapy may help eliminate residual cells at the site of the primary tumor and/or cells that have disseminated.

“Neoadjuvant therapy” refers to adjuvant therapy administered prior to local therapy, e.g., to shrink a primary tumor.

“Anti-HER2” therapy refers to administration of an antitumor agent that acts primarily by interacting with (e.g., interfering with) HER2. Such agents may be referred to as “anti-HER2” agents. Anti-HER2 agents include, e.g., monoclonal antibodies that bind to HER2, such as trastuzumab and pertuzumab, and various small molecule kinase inhibitors that bind to HER2 and inhibits its kinase activity. Pertuzumab is a recombinant, humanized monoclonal antibody that binds to the extracellular domain II, sterically blocking homo- and heterodimerization with other ERBB receptors, thus preventing signal transduction. In some embodiments, an anti-HER2 agent inhibits HER2 and at least one other member of the human epidermal growth factor receptor family. Examples of such agents include, e.g., dual EGFR (Erb-B1) and HER2 kinase inhibitors such as lapatinib and pan-ERBB kinase inhibitors such as neratinib. In some embodiments, an anti-tumor agent is an antibody-drug conjugate (ADC). For example, an anti-HER2 antibody can be conjugated to a cytotoxic agent. Cytotoxic agents useful for such purposes include, e.g., calicheamicins, auristatins, maytansinoids, and derivatives of CC 1065. For example, trastuzumab emtansine (T-DM1) is an antibody-drug conjugate ADC that combines intracellular delivery of the cytotoxic agent, DM1 (a derivative of maytansine) with the antitumor activity of trastuzumab.

In some embodiments, results of an HSF1-based assay may be useful for selecting an appropriate treatment regimen and/or for selecting the type or frequency of procedures to be used to monitor the subject for local or metastatic recurrence after therapy and/or the frequency with which such procedures are performed. For example, subjects classified as having a poor prognosis (being at high risk of poor outcome) may be treated and/or monitored more intensively than those classified as having a good prognosis. Thus any of the diagnostic, prognostic, or treatment-specific predictive methods can further comprise using information obtained from the assay to help in selecting a treatment or monitoring regimen for a subject suffering from cancer or at increased risk of cancer or at risk of cancer recurrence or in providing an estimate of the risk of poor outcome such as cancer related mortality or recurrence. The information may be used, for example, by a subject's health care provider in selecting a treatment or in treating a subject. A health care provider could also or alternatively use the information to provide a cancer patient with an accurate assessment of his or her prognosis. In some embodiments, a method of the invention can comprise making a treatment selection or administering a treatment based at least in part on the result of an HSF1-based assay. In some embodiments, a method of the invention can comprise selecting or administering more aggressive treatment to a subject, if the subject is determined to have a poor prognosis. In some embodiments, a method of the invention can comprise selecting or administering more aggressive treatment, if the subject is determined to have CIS that is positive for HSF1 expression or HSF1 activation. Often a “treatment” or “treatment regimen” refers to a course of treatment involving administration of an agent or use of a non-pharmacological therapy multiple times over a period of time, e.g., over weeks or months. A treatment can include one or more pharmacological agents (often referred to as “drugs” or “compounds”) and/or one or more non-pharmacological therapies such as radiation, surgery, etc. A treatment regimen can include the identity of agents to be administered to a subject and may include details such as the dose(s), dosing interval(s), number of courses, route of administration, etc. “Monitoring regimen” refers to repeated evaluation of a subject over time by a health care provider, typically separated in time by weeks, months, or years. The repeated evaluations can be on a regular or predetermined approximate schedule and are often performed with a view to determining whether a cancer has recurred or tracking the effect of a treatment on a tumor or subject.

“More aggressive” treatment (also referred to as “intensive” or “more intensive” treatment herein) can comprise, for example, (i) administration of chemotherapy in addition to, or instead of, hormonal therapy; (ii) administration of a dose of one or more agents (e.g., chemotherapeutic agent) that is at the higher end of the acceptable dosage range (e.g., a high dose rather than a medium or low dose, or a medium dose rather than a low dose) and/or administration of a number of doses or a number of courses at the higher end of the acceptable range and/or use of non-hormonal cytotoxic/cytostatic chemotherapy; (iii) administration of multiple agents rather than a single agent; (iv) administration of more, or more intense, radiation treatments; (v) administration of a greater number of agents in a combination therapy; (vi) use of adjuvant therapy; (vii) more extensive surgery, such as mastectomy rather than breast-conserving surgery such as lumpectomy. For example, a method can comprise (i) selecting that the subject not receive chemotherapy (e.g., adjuvant chemotherapy) if the tumor is considered to have a good prognosis; or (ii) selecting that the subject receive chemotherapy (e.g., adjuvant chemotherapy), or administering such chemotherapy, if the tumor is considered to have a poor prognosis. In some embodiments, a method of the invention can comprise selecting that a subject receives less aggressive treatment or administering such treatment, if the subject is determined to have a good prognosis. “Less aggressive” (also referred to as “less intensive”) treatment could entail, for example, using dose level or dose number at the lower end of the acceptable range, not administering adjuvant therapy, selecting a breast-conserving therapy rather than mastectomy, selecting hormonal therapy rather than non-hormonal cytotoxic/cytostatic chemotherapy, or simply monitoring the patient carefully. “More intensive” or “intensive” monitoring could include, for example, more frequent clinical and/or imaging examination of the subject or use of a more sensitive imaging technique rather than a less sensitive technique. “Administering” a treatment could include direct administration to a subject, instructing another individual to administer a treatment to the subject (which individual may be the subject themselves in the case of certain treatments), arranging for administration to a subject, prescribing a treatment for administration to a subject, and other activities resulting in administration of a treatment to a subject. “Selecting” a treatment or treatment regimen could include determining which among various treatment options is appropriate or most appropriate for a subject, recommending a treatment to a subject, or making a recommendation of a treatment for a subject to the subject's health care provider.

In some aspects, the invention provides a method of selecting a regimen for monitoring or treating a subject in need of treatment for cancer comprising: (a) assessing the level of HSF1 expression or HSF1 activation in a sample obtained from the subject; and (b) selecting an intensive monitoring or treatment regimen if the level of HSF1 expression or HSF1 activation is increased in the sample. In some aspects, the invention provides a method of selecting a regimen for monitoring or treating a subject in need of treatment for cancer, wherein said regimen is selected from among multiple options including at least one more intensive regimen and at least one less intensive regimen, the method comprising: (a) obtaining a classification of the subject, wherein the subject is classified into a high risk or a low risk group based at least in part on an assessment of the level of HSF1 expression or HSF1 activation in a sample obtained from the subject; and (b) selecting a more intensive regimen if the subject is classified as being in a high risk group or selecting a less intensive regimen if the subject is classified as being in a low risk group. In some aspects, the invention provides a method of monitoring or treating a subject in need of treatment for cancer comprising: (a) obtaining a classification of the subject, wherein the classification is based at least in part on an assessment of the level of HSF1 expression or HSF1 activation in a sample obtained from the subject; and (b) monitoring or treating the subject according to an intensive regimen if the subject is classified as being in a high risk group or monitoring or treating the subject with a less intensive regimen if the subject is classified as being in a low risk group. “Obtaining a classification” could comprise any means of ascertaining a classification such as performing an HSF1-based assay (or directing that an HSF1-based assay be performed) and assigning a classification based on the results, receiving results of an HSF1-based assay and assigning a classification using the results, receiving or reviewing a classification that was previously performed, etc.

In some embodiments a subject has been previously treated for the cancer, while in other embodiments the subject has not previously received treatment for the cancer. In some embodiments the previous treatment for a breast tumor is hormonal therapy such as tamoxifen or another anti-estrogen agent, e.g., another SERM.

In some embodiments, a subject falls within a selected age group or range, e.g., 40 years old or less, 50 years old or less, 55 years old or less, 60 years old or less, between 40 and 60 years of age, 40 years old or more, 50 years old or more, 55 years old or more, 60 years old or more, etc. Any age group or range may be selected in various embodiments of the invention, whether or not specifically mentioned here. In some embodiments, a female subject is pre-menopausal. In some embodiments, a female subject is post-menopausal.

In some embodiments a subject, e.g., a subject having or at risk of lung cancer or lung cancer recurrence, is a current smoker or former smoker. In some embodiments a subject, e.g., a subject having or at risk of developing lung cancer or lung cancer recurrence, is a non-smoker who has no or essentially no history of smoking.

In some embodiments, an HSF1-based method may be used to identify cancer patients that do not require adjuvant therapy, e.g., adjuvant hormonal therapy and/or adjuvant chemotherapy. For example, a prognostic method may identify patients that have a good prognosis and would be unlikely to experience clinically evident recurrence and/or metastasis even without adjuvant therapy. Since adjuvant therapy can cause significant side effects, it would be beneficial to avoid administering it to individuals whom it would not benefit. In some embodiments, an HSF1-based prognostic method of the invention may be used to identify cancer patients that have a poor prognosis (e.g., they are at high risk of recurrence and/or metastasis) and may therefore benefit from adjuvant therapy. In some embodiments, an HSF1-based prognostic method may be used to identify cancer patients that might not be considered at high risk of poor outcome based on other prognostic indicators (and may therefore not receive adjuvant therapy) but that are in fact at high risk of poor outcome, e.g., recurrence and/or metastasis. Such patients may therefore benefit from adjuvant therapy. In some embodiments, HSF1-based method may be used in a subject with cancer in whom an assessment of the tumor based on standard prognostic factors, e.g., standard staging criteria (e.g., TMN staging), histopathological grade, does not clearly place the subject into a high or low risk category for recurrence after local therapy (e.g., surgery) and/or for whom the likelihood of benefit from adjuvant therapy is unclear, as may be the case in various early stage cancers where, e.g., the cancer is small and has not detectably spread to regional lymph nodes or metastasized more remotely.

In some embodiments, an HSF1-based method may be used to provide prognostic information for a subject with a breast tumor that has one or more recognized clinicopathologic features and/or that falls into a particular class or category based on gene expression profiling. For example, breast cancers can be classified into molecular subtypes based on gene expression profiles, e.g., luminal A, luminal B, ERBB2-associated, basal-like, and normal-like (see, e.g., Serlie, T., et al., Proc Natl Acad Sci USA. (2001) 98(19):10869-74). Breast cancers can be classified based on a number of different clinicopathologic features such as histologic subtype (e.g., ductal; lobular; mixed), histologic grade (grade 1, 2, 3); estrogen receptor (ER) and/or progesterone receptor (PR) status (positive (+) or negative (−)), HER2 (ERBB2) expression status, and lymph node involvement. For example, the following breast cancer subtypes can be defined based on expression of estrogen receptor (ER) and human epidermal growth factor receptor 2 (HER2), e.g., as assessed by immunohistochemistry (IHC): (1) ER+, HER2+; (2) ER+, HER2; (3) ER−, HER2+; and (4) ER−, HER2−. The level of expression can be used to further divide these subtypes. Amplification of the HER2 locus can be assessed, e.g., using in situ hybridization (ISH), e.g., fluorescent in situ hybridization (FISH). In some embodiments, an HSF1-based method is applied to a tumor that is ER+. In some embodiments an HSF1-based method is applied to a tumor that is ER−. In some embodiments an HSF1-based method is applied to a tumor that is HER2+. In some embodiments an HSF1-based method is applied to a tumor that is HER2−. In some embodiments an HSF1-based method is applied to a tumor that is PR+. In some embodiments an HSF1-based method is applied to a tumor that is PR−. In some embodiments an HSF1-based method is applied to a tumor that is EGFR+. In some embodiments an HSF-based method is applied to a tumor that is EGFR−. It will be understood that these markers may be present or absent in any combination in various embodiments. For example, in some embodiments an HSF1-based method is applied to a tumor that is ER+/HER2+ or ER+/HER2− (each of which categories can include tumors that are PR+ or PR− and are EGFR+ or EGFR−). In some embodiments, the sample or tumor is not “triple negative”, i.e., the sample or tumor is negative for expression of ER, PR, and HER2.

In some embodiments a subject has DCIS. In some embodiments a subject has Stage I or Stage II breast cancer. In some embodiments a subject has Stage III breast cancer. In some embodiments, cancer stage is assigned using pathologic criteria, clinical criteria, or a combination of pathologic and clinical criteria.

In some embodiments a subject does not have detectable lymph node involvement, i.e., the subject is “lymph node negative” (LNN). For example, the subject may have be ER+/lymph node negative. The clinical management of subjects in this early stage group (e.g., treatment selection) is challenging due to the lack of markers indicating which small portion of the population will have a recurrence (e.g., following surgery) and could therefore benefit from more intensive monitoring and/or more aggressive treatment. In accordance with certain embodiments of the invention, a subject with ER+, LNN cancer that has increased HSF1 expression or increased HSF1 activation is monitored and/or treated more intensively than if the cancer does not have increased HSF1 expression or increased HSF1 activation.

In some embodiments, increased HSF1 expression or increased HSF1 activation in a sample from an ER+ breast tumor identifies patients having ER+ tumors that may be resistant to hormonal therapy. Such patients may benefit from use of a more aggressive treatment regimen, e.g., chemotherapy in addition to, or instead of, hormonal therapy, or more extensive surgery.

It has been reported that while about half of all breast cancers are assigned histologic grade 1 or 3 status (with a low or high risk of recurrence, respectively), a substantial percentage of tumors (30%-60%) are classified as histologic grade 2, which is less informative for clinical decision making because of intermediate risk of recurrence (Sotiriou C, et al., J Natl Cancer Inst., 98(4):262-72, 2006). Improved prognostic methods could be of significant use in this setting, for example. In some embodiments, an HSF1-based method is applied to a tumor classified as histologic grade 2, e.g., to classify histologic grade 2 tumors into high and low risk groups. In some embodiments, an HSF1-based method is applied to a tumor classified as histologic grade 2, e.g., to classify histologic grade 2 tumors into higher and lower risk groups, wherein tumors that have increased HSF1 expression or HSF1 activation are classified into the higher risk group. Tumors that do not have increased HSF1 expression or HSF1 activation would be classified into the lower risk group.

In some embodiments, an HSF1-based assay is used to provide sample classification, diagnostic, prognostic, or treatment-predictive information pertaining to lung cancer, e.g., non-small cell lung cancer (NSCLS), such as a lung adenocarcinoma. In some embodiments, the lung cancer, e.g., lung adenocarcinoma, is a Stage I cancer (T1 N0 M0 or T2 N0 M0). In some embodiments the cancer is a Stage 1A lung cancer (T1 N0 M0). In some embodiments the cancer is a Stage IB lung cancer (T1N0M0). In some embodiments, the lung cancer, e.g., lung adenocarcinoma, is a Stage II cancer. Stage I and II lung cancers are typically treated by surgical resection of the tumor. Although surgery can be curative, a significant fraction of patients develop recurrence or metastases. Such patients might benefit from adjuvant therapy (radiation and/or chemotherapy). However, the current standard staging system (TMN) cannot predict which stage I or II lung cancers will recur. Although studies have shown adjuvant chemotherapy to be of benefit in groups of patients with stage II lung cancer, its role in treating stage I lung cancer is unclear. Without wishing to be bound by any theory, the number of patients diagnosed with stage I or II lung cancer may increase significantly at least in part due to the increased use of imaging modalities such as computed tomography (CT) scans for screening purposes, e.g., in individuals who have a significant smoking history. It would be useful to be able to identify those patients with stage I or stage II cancer who are at increased likelihood of recurrence and may therefore be more likely to benefit from adjuvant chemotherapy. In some embodiments, an HSF1-based method is applied to classify a stage I or stage II lung tumor into a higher or lower risk group, wherein tumors that have increased (e.g., high or intermediate) HSF1 expression or HSF1 activation are classified into the higher risk group. Tumors that have absent or low HSF1 expression or HSF1 activation are classified into the lower risk group. Subjects with tumors classified into the higher risk group have an increased likelihood of recurrence than subjects with tumors classified into the lower risk group and may benefit from adjuvant chemotherapy. Subjects with tumors classified into the lower risk group may be treated with surgery alone. Adjuvant chemotherapy for operable lung cancer frequently includes a platinum-based agent (e.g., cisplatin or carboplatin), optionally in combination with an anti-mitotic agent (e.g., an anti-microtubule agent) such as a taxane (e.g., paclitaxel (Taxol) or docetaxel (Taxotere)) or a vinca alkaloid such as vinblastine, vincristine, vindesine and vinorelbine. Other agents that may be administered as adjuvant chemotherapy in operable lung cancer, typically in combination with a platinum agent, include mitomycin, doxorubicin, or etoposide. Other adjuvant chemotherapy regiments include tegafur alone, uracil alone, a combination of tegafur and uracil, or a combination of tegafur and/or uracil with a platinum agent.

In some embodiments a subject has been previously treated for the cancer, while in other embodiments the subject has not previously received treatment for the cancer. In some embodiments the previous treatment for a breast tumor is hormonal therapy such as tamoxifen or another anti-estrogen agent, e.g., another SERM.

In some embodiments, a subject falls within a selected age group or range, e.g., 40 years old or less, 50 years old or less, 55 years old or less, 60 years old or less, between 40 and 60 years of age, 40 years old or more, 50 years old or more, 55 years old or more, 60 years old or more, etc. Any age group or range may be selected in various embodiments of the invention, whether or not specifically mentioned here. In some embodiments, a female subject is pre-menopausal. In some embodiments, a female subject is post-menopausal.

In some embodiments a subject, e.g., a subject having or at risk of lung cancer or lung cancer recurrence, is a current smoker or former smoker. In some embodiments a subject, e.g., a subject having or at risk of developing lung cancer or lung cancer recurrence, is a non-smoker who has no or essentially no history of smoking.

Any method of the invention that comprises assessing HSF1 expression or HSF1 activation or using the level of expression or activation of an HSF1 gene product may, in certain embodiments, further comprise assessing or using the level of expression, activation, or activity of one or more additional cancer biomarkers. Any method of the invention that comprises assessing HSF1-CP expression or using the level of expression of one or more HSF1-CP gene products may, in certain embodiments, further comprise assessing or using the level of expression, activation, or activity of one or more additional cancer biomarkers. In certain embodiments, the level of expression, activation, or activity of an HSF1 gene product and/or an HSF1-CP gene product is used in conjunction with the level of expression, activation, or activity of one or more additional cancer biomarkers in a method of providing diagnostic, prognostic, or treatment-specific predictive information. The additional cancer biomarker(s) may be selected based at least in part on the site in the body from which a sample was obtained or the suspected or known tissue of origin of a tumor. For example, in the case of suspected or known breast cancer, one or more breast cancer biomarkers may be assessed.

In some embodiments, an HSF1-based assay is used together with additional information, such as results of a second assay (or multiple assays) and/or clinicopathological information to provide diagnostic, prognostic, or treatment-predictive information pertaining to breast cancer. In some embodiments, such information comprises, e.g., subject age, tumor size, nodal involvement, tumor histologic grade, ER status, PR status, and/or HER2 status, menopausal status, etc.). In some embodiments, the additional information includes the PR status of the tumor. For example, a method can comprise determining the PR status of a tumor and, if the PR status is positive, classifying the tumor with respect to prognosis or treatment selection based on expression of HSF1 or activation of HSF1. In some embodiments, information from an HSF1-related assay is used together with a decision making or risk assessment tool such as the computer program Adjuvant! Online (https://www.adjuvantonline.com/index.jsp). The basic format of an early version of Adjuvant! was described in the article Ravdin, Siminoff, Davis, et al. JCO 19(4) 980-991, 2001. In some embodiments, the second assay is a gene expression profiling assay such as the MammaPrint® (Agendia BV, Amsterdam, the Netherlands), Oncotype DX™ (Genomic Health, Redwood City, Calif.), Celera Metastasis Score™ (Celera, Inc., Rockville, Md.), Breast BioClassifier (ARUP, Salt Lake City, Utah), Rotterdam signature 76-gene panel (Erasmus University Cancer Center, Rotterdam, The Netherlands), MapQuant Dx™ Genomic Grade test (Ipsogen, Stamford, Conn.), Invasiveness Gene Signature (OncoMed Pharmaceuticals, Redwood City, Calif.), NuvoSelect™ assay (Nuvera Biosciences, Woburn, Mass.), THEROS Breast Cancer IndexSM (BCI) (bioTheranostics, San Diego) that classifies tumors (e.g., into high or low risk groups) based on expression level of multiple genes using, e.g., a microarray or multiplex RT-polymerase chain reaction (PCR) assay. The phrase “used together” with in regard to two or more assays means that the two or more assays are applied to a particular tumor. In some embodiments, the two or more assays are applied to the same sample (or a portion thereof) obtained from the tumor.

In some embodiments, an HSF1-based assay may be used together with a gene expression profile in which expression level of at least 1, at least 5, or at least 10 different genes (“classifier genes”) is used to classify a tumor. It will be understood that such gene expression profile assays may measure expression of control genes as well as classifier genes. In some embodiments an HSF1-based assay is used together with an H:I™ test (bioTheranostics, Carlsbad, Calif.), in which the ratio of expression of HOXB 13 and IL-17B genes is used to classify a tumor. In some embodiments, an HSF1-based assay is used together with an antibody-based assay, e.g., the ProEx™ Br (TriPath Oncology, Durham, N.C.), Mammostrat® (Applied Genomics, Inc., Huntsville, Ala.), ADH-5 (Atypical Ductal Hyperplasia) Breast marker antibody cocktail (Biocare Medical, Concord, Calif.), measurement of urokinase-like plasminogen activator (uPA) and/or its inhibitor plasminogen activator inhibitor 1 (PAI1), or a FISH-based test such as the eXaagenBC™ (eXagen Diagnostics, Inc., Albuquerque, N. Mex.). In some embodiments, an HSF1-based assay is used together with an assay that measures proliferation. For example, expression of a proliferation marker such as Ki67 (Yerushalmi et al., Lancet Oncol. (2010), 11(2):174-83) can be used. In some embodiments, an HSF1-based assay is used together with a miRNA-based assay (e.g., an assay that measures expression of one or more miRNAs or miRNA precursors). For example, in some embodiments, an HSF1-based assay is used together with a miR31-based assay, e.g., as described in PCT/US2009/067015 (WO/2010/065961).

An HSF1-based assay (e.g., any of the HSF1-based assays described herein) may be used together with another assay in any of a number of ways in various embodiments of the invention. For example, in some embodiments, if results of two tests are discordant (e.g., one test predicts that the subject is at high risk while the other predicts that the subject is at low risk), the subject may receive more aggressive therapeutic management than if both tests predict low risk. In some embodiments, if a result of a non-HSF1-based assay is inconclusive or indeterminate, an HSF1-based assay can be used to provide a diagnosis, prognosis, or predictive information. In some embodiments, one can have increased confidence if results of an HSF1-based assay and a second assay are in agreement. For example, if both tests indicate that the subject is at low risk, there can be increased confidence in the appropriateness of providing less aggressive therapeutic management, e.g., to not administer adjuvant chemotherapy, while if both tests indicate that the subject is at high risk, there can be increased confidence in the appropriateness of providing more aggressive therapeutic management.

In some embodiments, a method of the invention comprises providing treatment-specific predictive information relating to use of a proteostasis modulator to treat a subject with cancer, based at least in part on assessing the level of expression of HSF1 or activation of HSF1 in a sample obtained from the subject. “Proteostasis” (which term is used interchangeably with “protein homeostasis”) refers to controlling the concentration, conformation (e.g., folding), binding interactions (quaternary structure), and subcellular location of the proteins within a cell, often through mechanisms such as transcriptional and/or translational changes, chaperone-assisted folding and disaggregation, or controlled protein degradation. Proteostasis can be thought of as a network comprising multiple distinguishable pathways (“proteostasis pathways”) that may interact with and influence each other. Proteostasis pathways include, e.g., the HSR (discussed above), the ubiquitination-proteasome degradation pathway, and the unfolded protein response (UPR). “Proteostasis modulator” refers to an agent that modulates one or more proteostasis pathways.

In some embodiments, a sample can be classified as belonging to (i.e., obtained from) a subject with cancer who is a suitable candidate for treatment with a proteostasis modulator. For example, the invention provides a method of determining whether a subject with cancer is a suitable candidate for treatment with a proteostasis modulator, comprising assessing the level of HSF1 expression or HSF1 activation in a sample obtained from the subject, wherein an increased level of HSF1 expression or an increased level of HSF1 activation in the sample is indicative that the subject is a suitable candidate for treatment with a proteostasis modulator. In some embodiments, the invention provides a method of determining whether a subject with cancer is likely to benefit from treatment with a proteostasis modulator, comprising: assessing the level of HSF1 expression or HSF1 activation in a sample obtained from the subject, wherein an increased level of HSF1 expression or an increased level of HSF1 activation in the sample is indicative that the subject is likely to benefit from treatment with a proteostasis modulator. In some embodiments, the invention provides a method of identifying a subject with cancer who is likely to benefit from treatment with a proteostasis modulator, comprising assessing the level of HSF1 expression or HSF1 activation in a sample obtained from the subject, wherein an increased level of HSF1 expression or an increased level of HSF1 activation in the sample identifies the subject as being likely to benefit from treatment with a proteostasis modulator. In some embodiments, the invention provides a method of predicting the likelihood that a tumor will be sensitive to a protein homeostasis modulator, the method comprising: assessing the level of HSF1 expression or the level of HSF1 activation in a sample obtained from the tumor; wherein if the level of HSF1 expression or activation is increased, the tumor has an increased likelihood of being sensitive to the protein homeostasis modulator. A tumor is “sensitive” to a treatment if the subject experiences a partial or complete response or stabilization of disease following treatment. Response can be assessed, for example, by objective criteria such as anatomical tumor burden, as known in the art. In some embodiments, a response correlates with increased progression-free survival or increased overall survival. Thus in some embodiments, a tumor is sensitive to a treatment if administration of the treatment correlates with increased progression-free survival or increased overall survival.

In some embodiments, treatment with a proteostasis modulator comprises administering a proteostasis modulator to the subject in addition to a standard treatment regimen for treating the subject's cancer. It will be understood that the proteostasis modulator is typically administered in an effective amount in a suitable pharmaceutical composition that may comprise one or more pharmaceutically acceptable carriers. “Pharmaceutically acceptable carrier” refers to a diluent, excipient, or vehicle with which the therapeutically active agent is administered. An effective amount may be administered in one dose or multiple doses.

Without wishing to be bound by any theory, increased HSF1 activity may help tumor cells cope with the stress of therapy (e.g., pharmacological agents, radiation, etc.) and/or may promote phenotypic diversity among tumor cells by helping tumor cells cope with the consequences of mutations. Such effects may contribute to poor outcomes in cancer by, for example, promoting emergence of malignant or more aggressive tumor subclones and/or promoting treatment resistance. Administration of a proteostasis modulator may counteract such effects. In some embodiments, a therapeutic benefit could result at least in part from a proteostasis modulator reducing the likelihood that a tumor will become resistant to such treatment or at least in part reversing resistance that may be present at the time of treatment. For example, addition of a proteostasis modulator to a standard chemotherapy or hormonal regimen for breast cancer may reduce the likelihood that a tumor will become resistant to such regimen, or at least in part reverse resistance that may be present at the time of treatment. Based at least in part on the discovery that HSF1 expression and HSF1 activation are increased in pre-invasive cancer, the invention encompasses the recognition that intervention at the pre-invasive stage of cancer with a proteostasis modulator (e.g., to counteract HSF1's activity) may delay or reduce the likelihood of progression to invasive cancer. In some aspects, the invention encompasses the recognition that treatment of subjects without evidence of cancer (e.g., subjects at increased risk of cancer) with a proteostasis modulator (e.g., to counteract HSF1's activity) may inhibit or reduce the likelihood that the subject will develop cancer. It should be noted that a subject may be a suitable candidate for treatment with a proteostasis modulator even if the tumor does not exhibit increased HSF1 expression or increased HSF1 activation. For example, subjects with early stage cancer that has not progressed to a state in which HSF1 is activated may benefit

In some aspects, the invention provides a method of treating a subject who has pre-invasive cancer, the method comprising administering a proteostasis modulator to a subject with pre-invasive cancer. Such treatment may, for example, inhibit progression of the pre-invasive cancer to invasive cancer. In some aspects, the invention provides a method of treating a subject at increased risk of cancer, the method comprising administering a proteostasis modulator to the subject. In some aspects, the invention provides a method of inhibiting development of cancer in a subject, the method comprising administering a proteostasis modulator to the subject.

In some aspects, the invention provides a method of inhibiting recurrence of cancer in a subject, the method comprising administering a proteostasis modulator to the subject. In some embodiments, the cancer is characterized by increased HSF1 expression or increased HSF1 activation.

In some aspects, the invention provides a method of inhibiting emergence of resistance to therapy in a subject with cancer, the method comprising administering a proteostasis modulator to the subject in combination with an additional therapy, thereby reducing the likelihood of resistance to the additional therapy. In some embodiments, the additional therapy is a chemotherapeutic agent. In some embodiments, the additional therapy is a hormonal agent. In some embodiments, the cancer is characterized by increased HSF1 expression or increased HSF1 activation.

In some embodiments, a proteostasis modulator is an HSR modulator, e.g., an HSR inhibitor. “HSR inhibitor” refers to an agent that inhibits expression or activity of at least one component of the HSR. HSR components include, e.g., HSF1 itself and heat shock proteins such as HSP 40, HSP70, and HSP90. In some embodiments, the component of the HSR is HSP90. For purposes of the present invention, HSP90 refers to HSP90A family HSP90, commonly referred to in the art as “cytoplasmic HSP90” (see Taipale, M, et al., Nat. Rev. Mol. Cell. Biol. (2010) 11(7):515-28 for review). Most vertebrates, including humans, have two genes encoding HSP90A proteins with very similar sequences and highly overlapping functions: HSP90AA1 (Gene ID for human gene: 3320; Gene ID for mouse ortholog: 15519) and HSP90AB1 (Gene ID for human gene: 3326; Gene ID for mouse gene: 15516). The proteins encoded by HSP90AA1 and HSP90AB1 are referred to as HSP90a and HSP90β, respectively. For purposes of the present invention, an “HSP90 inhibitor” refers to a compound that inhibits at least one HSP90A, e.g., HSP90β. In some embodiments, the compound inhibits both HSP90α and HSP90β. HSP90A is an ATPase and contains three main structural domains: a highly conserved N-terminal (NTD) domain of ˜25 kDa, which contains a binding pocket for ATP; a middle domain (MD) of ˜40 kDa, and a C-terminal domain (CTD) of ˜12 kDa. HSP90A forms homodimers and undergoes a dynamic cycle termed the “chaperone cycle” involving ATP binding and hydrolysis, during which it undergoes conformational shifts that are important in its recognition and release of client proteins.

Numerous HSP90 inhibitors are known in the art. In general, an HSP90 inhibitor can inhibit HSP90 activity in any of a variety of ways, such as by inhibiting the ATPase activity of HSP90. In some embodiments an HSP90 inhibitor specifically binds to the ATP binding pocket of HSP90. In some embodiments an HSP90 inhibitor binds outside the ATP binding pocket. A number of HSP90 inhibitors have shown promise in the treatment of cancer, and others are under investigation. Exemplary HSP90 inhibitors include, e.g., benzoquinone ansamycins such as geldanamycin and herbimycin, resorcylic acid lactones such as radicicol, purine scaffold compounds, and a variety of synthetic compounds based on other chemical scaffolds (see, e.g., Taldone, T., et al. Bioorg Med Chem., 17(6):2225-35, 2009 or Trepel, J., et al., Nat Rev Cancer. 10(8):537-49, 2010). Exemplary HSP90 inhibitors that have entered clinical development (i.e., they have been administered to at least one human subject in a clinical trials) include, e.g., geldanamycin analogs such as 17-allylamino-17-demethoxygeldanamycin (17-AAG, also called tanespimycin), 17-dimethylaminoethylamino-17-demethoxygeldanamycin (I 7-DMAG), retaspimycin (IPI-504), alvespimycin (IPI-493), SNX-5422, AUY922, STA-9090, HSP990, CNF2024 (BIIB021), XL888, AT13387, and MPC-3100.

An ongoing challenge in the development of HSP90 inhibitors has been the identification of which patients are likely to benefit from treatment with these drugs (36-39). The basal level of HSP90 in tumors per se has generally not proven to be predictive. Without wishing to be bound by any theory, the effectiveness of HSF1, even as a single marker, in predicting the outcome of cancers as described herein may reflect the fact that HSF1, as a dominant regulator of the entire heat shock network, serves as a better indicator of the overall stress levels within a tumor and consequently the “load” on the HSP-based chaperone machinery. In accordance with certain aspects of the invention, this load could determine which patients might benefit from a HSP90 inhibitor, either alone or in combination with other agents. In some embodiments, the HSP90 inhibitor has entered clinical development for, e.g., treatment of cancer. In some embodiments the HSP90 inhibitor is a small molecule.

In some embodiments, a proteostasis modulator is an HSF1 inhibitor. As used herein, an “HSF1 inhibitor” is an agent that inhibits expression or activity of HSF1. In some embodiments, an HSF1 inhibitor is an RNAi agent, e.g., a short interfering RNA (siRNA) or short hairpin RNA (shRNA) that, when present in a cell (e.g., as a result of exogenous introduction of an siRNA or intracellular expression of a shRNA) results in inhibition of HSF expression by RNA interference (e.g., by causing degradation or translational repression of mRNA encoding HSF1, mediated by the RNAi-induced silencing complex). Exemplary RNAi agents that inhibit HSF1 expression are disclosed, e.g., in PCT/EP2010/069917 (WO/2011/073326) or in reference 6. In some embodiments an HSF1 inhibitor may be an intrabody that binds to HSF1, or an agent such as a single chain antibody, aptamer, or dominant negative polypeptide that binds to HSF1, wherein the agent optionally comprises a moiety that allows it to gain entry into tumor cells. For example, the agent may comprise a protein transduction domain that allows the agent to cross the plasma membrane or a ligand that binds to a cell surface receptor such that the agent is internalized, e.g., by endocytosis. In some embodiments the HSF1 inhibitor comprises a small molecule. In some embodiments the HSF1 inhibitor comprises an agent that inhibits activation of HSF1. For example, the agent may at least in part block assembly of multimers, e.g., trimers, comprising HSF1. Suitable agents for inhibiting HSF1 may be identified using a variety of screening strategies.

In some embodiments, a proteostasis modulator is a proteasome inhibitor. The proteasome is a large, multi-protein complex that unfolds and proteolyses substrate polypeptides, reducing them to short fragments (Lodish, et al., supra). Most protein degradation by the proteasome occurs via the ubiquitination-proteasome degradation pathway (UPD pathway), a multistep enzymatic cascade in eukaryotes in which ubiquitin is conjugated via a lysine residue to target proteins for destruction. Proteins tagged with lysine-linked chains of ubiquitin are marked for degradation in the proteasome. Proteasome-mediated protein degradation, e.g., via the UPD pathway, allows cells to eliminate excess and misfolded proteins and regulates various biological processes, such as cell proliferation. “Proteasome inhibitor” refers to an agent that inhibits activity of the proteasome or inhibits synthesis of a proteasome component. Proteasome inhibitors include, e.g., a variety of peptidic and non-peptidic agents that bind reversibly to the proteasome, bind covalently to the active site of the proteasome, or bind to the proteasome outside the active site (sometimes termed “allosteric inhibitors”) (Ruschak A M, et al., J Natl Cancer Inst. (2011) 103(13):1007-17). A number of proteasome inhibitors have shown promise in the treatment of cancer, including bortezomib (Velcade®) (approved by the US FDA), and various others under investigation. Exemplary proteasome inhibitors that have been tested in clinical trials in cancer include bortezomib, CEP-18770, MLN-9708, carfilzomib, ONX 0912, and NPI-0052 (salinosporamide A). HIV protease inhibitors such as nelvinavir also inhibit the proteasome. Other agents that inhibit the proteasome include chloroquine, 5-amino-8-hydroxyquinoline (5AHQ), disulfiram, tea polyphenols such as epigallocatechin-3-gallate, MG-132, PR-39, PS-I, PS-IX, and lactacystin. In some embodiments, a method of the invention is applied with regard to proteasome inhibitor that has entered clinical development for, e.g., treatment of cancer.

In some aspects, the invention encompasses use of a method comprising assessing the level of HSF1 expression or HSF1 activation as a “companion diagnostic” test to determine whether a subject is a suitable candidate for treatment proteostasis modulator. In some embodiments a proteostasis modulator may be approved (allowed to be sold commercially for treatment of humans or for veterinary purposes) by a government regulatory agency (such as the US FDA, the European Medicines Agency (EMA), or government agencies having similar authority over the approval of therapeutic agents in other jurisdictions) with the recommendation or requirement that the subject is determined to be a suitable candidate for treatment with the proteostasis modulator based at least in part on assessing the level of HSF1 expression or HSF1 activation in a tumor sample obtained from the subject. For example, the approval may be for an “indication” that includes the requirement that a subject or tumor sample be classified as having high levels or increased levels of HSF1 expression or HSF1 activation. Such a requirement or recommendation may be included in the package insert provided with the agent. In some embodiments a particular method for detection or measurement of an HSF1 gene product or of HSF1 activation or a specific test reagent (e.g., an antibody that binds to HSF1 polypeptide or a probe that hybridizes to HSF1 mRNA) or kit may be specified. In some embodiments, the method, test reagent, or kit will have been used in a clinical trial whose results at least in part formed the basis for approval of the proteostasis modulator. In some embodiments, the method, test reagent, or kit will have been validated as providing results that correlate with outcome of treatment with the proteostasis modulator.

In some aspects, the invention provides a method of assessing efficacy of treatment of cancer comprising: (a) assessing the level of HSF1 expression or HSF1 activation in a sample obtained from a subject that has been treated for cancer, wherein absence of increased HSF1 expression or increased HSF1 activation in said sample indicates effective treatment. In some embodiments, step (a) is repeated at one or more time points following treatment of the subject for cancer, wherein continued absence of increased HSF1 expression or increased HSF1 activation of over time indicates effective treatment. The sample may be obtained, for example, from or close to the site of a cancer that was treated (e.g., from or near a site from which a tumor was removed).

In some aspects, the invention provides a method of assessing efficacy of treatment of cancer comprising: (a) assessing the level of HSF1 expression or HSF1 activation in a sample obtained from a subject having cancer, and (b) repeating step (a) at one or more time points during treatment of the subject for cancer, wherein decreased HSF1 expression or decreased HSF1 activation of over time indicates effective treatment. The sample may be obtained, for example, from or close to the site of a cancer being treated.

In some aspects, the invention provides a method of monitoring a subject for cancer recurrence comprising: (a) assessing the level of HSF1 expression or HSF1 activation in a sample obtained from a subject that has been treated for cancer, wherein presence of increased HSF1 expression or increased HSF1 activation in the sample indicates cancer recurrence. In some embodiments, step (a) is repeated at one or more time points following treatment of the subject for cancer. The sample may be obtained, for example, from or close to the site of a cancer that was treated (e.g., from or near a site from which a tumor was removed).

In certain embodiments of any aspect of the invention, a cancer is breast cancer. In certain aspects, the invention provides the recognition that assessment of HSF1 expression or activation for diagnostic, prognostic, or predictive purposes may be of particular use in estrogen receptor (ER) positive breast cancer. In certain embodiments of any of the inventive methods relating to breast cancer, the breast cancer is estrogen receptor (ER) positive breast cancer.

Certain aspects and embodiments of the invention are described herein mainly in regard to breast cancer (e.g., breast tumor cells, breast tumor samples, breast tumors, and/or subjects in need of prognosis, diagnosis, or treatment selection for breast cancer). It will be understood that the invention encompasses embodiments in which products and processes described herein are applied in the context of tumors arising from organs or tissues other than the breast. One of ordinary skill in the art will recognize that certain details of the invention may be modified according, e.g., to the particular tumor type or tumor cell type of interest. Such embodiments are within the scope of the invention.

It will be understood that many of the methods provided herein, e.g., methods of classification, may be described in terms of samples, tumors, or subjects and such descriptions maybe considered equivalent and freely interchangeable. For example, where reference is made herein to a method of classifying a sample, such method may be expressed as a method of classifying a tumor from which the sample was obtained or as a method of classifying a subject from which the sample originated in various embodiments. Similarly, where reference is made herein to assessing the level of HSF1 expression or HSF1 activation in a sample, such method may be expressed as a method of assessing the level of HSF1 expression or HSF1 activation in a tumor from which the sample was obtained in various embodiments. It will also be understood that a useful diagnostic, prognostic, or treatment-specific predictive method need not be completely accurate. For example, “predicting”, “predicting the likelihood”, and like terms, as used herein, do not imply or require the ability to predict with 100% accuracy and do not imply or require the ability to provide a numerical value for a likelihood (although such value may be provided). Instead, such terms typically refer to forecast of an increased or a decreased probability that a result, outcome, event, etc., of interest exists or will occur, e.g., when particular criteria or conditions exist, as compared with the probability that such result, outcome, or event, etc., exists or will occur when such criteria or conditions are not met.

Methods of Assessing HSF1 Expression or HSF1 Activation

HSF1 genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Exemplary databases include, e.g., GenBank, RefSeq, Gene, UniProtKB/SwissProt, UniProtKB/Trembl, and the like. The HSF1 gene has been assigned NCBI GeneID: 3297. The NCBI Reference Sequence accession numbers for human HSF1 mRNA and polypeptide are NM_{—}005526 and NP_{—}005517, respectively, and the human HSF1 polypeptide GenBank acc. no. is AAA52695.1. The human HSF1 gene is located on chromosome 8 (8q24.3), RefSeq accession number NC_{—}000008.10. Sequences of other nucleic acids and polypeptides of interest herein could also be readily obtained from such databases. Sequence information may be of use, for example, to generate reagents for detection of HSF1 gene products.

In general, the level of HSF1 expression of HSF1 activation can be assessed using any of a variety of methods. In many embodiments, the level of HSF1 expression is assessed by determining the level of an HSF1 gene product in a sample obtained from a tumor. In some embodiments an HSF1 gene product comprises HSF1 mRNA. In general, any suitable method for measuring RNA can be used to measure the level of HSF1 mRNA in a sample. For example, methods based at least in part on hybridization and/or amplification can be used. Exemplary methods of use to detect mRNA include, e.g., in situ hybridization, Northern blots, microarray hybridization (e.g., using cDNA or oligonucleotide microarrays), reverse transcription PCR (e.g., real-time reverse transcription PCR), nanostring technology (see, e.g., Geiss, G., et al., Nature Biotechnology (2008), 26, 317-325; U.S. Ser. No. 09/898,743 (U.S. Pat. Pub. No. 20030013091) for exemplary discussion of nanostring technology and general description of probes of use in nanostring technology). A number of such methods include contacting a sample with one or more nucleic acid probe(s) or primer(s) comprising a sequence (e.g., at least 10 nucleotides in length, e.g., at least 12, 15, 20, or 25 nucleotides in length) substantially or perfectly complementary to a target RNA (e.g., HSF1 mRNA). The probe or primer is often detectably labeled using any of a variety of detectable labels. In many embodiments the sequence of the probe or primer is sufficiently complementary to HSF1 mRNA to allow the probe or primer to distinguish between HSF1 mRNA and most or essentially all (e.g., at least 99%/o, or more) transcripts from other genes in a mammalian cell, e.g., a human cell, under the conditions of an assay. In some embodiments, “substantially complementary” refers to at least 90% complementarity, e.g., at least 95%, 96%, 97%, 98%, or 99% complementarity. A probe or primer may also comprise sequences that are not complementary to HSF1 mRNA, so long as those sequences do not hybridize to other transcripts in a sample or interfere with hybridization to HSF1 mRNA under conditions of the assay. Such additional sequences may be used, for example, to immobilize the probe or primer to a support. A probe or primer may be labeled and/or attached to a support or may be in solution in various embodiments. A support may be a substantially planar support that may be made, for example, of glass or silicon, or a particulate support, e.g., an approximately spherical support such as a microparticle (also referred to as a “bead” or “microsphere”). In some embodiments, a sequencing-based approach such as serial analysis of gene expression (SAGE) (including variants thereof) or RNA-Sequencing (RNA-Seq) is used. RNA-Seq refers to the use of any of a variety of high throughput sequencing techniques to quantify RNA transcripts (see, e.g., Wang, Z., et al. Nature Reviews Genetics (2009), 10, 57-63). Other methods of use for detecting RNA include, e.g., electrochemical detection, bioluminescence-based methods, fluorescence-correlation spectroscopy, etc. It will be understood that certain methods that detect mRNA may, in some instances, also detect at least some pre-mRNA transcript(s), transcript processing intermediates, and degradation products of sufficient size.

In some embodiments an HSF1 gene product comprises HSF1 polypeptide. In general, any suitable method for measuring proteins can be used to measure the level of HSF1 polypeptide in a sample. In many embodiments, an immunological method or other affinity-based method is used. In general, immunological detection methods involve detecting specific antibody-antigen interactions in a sample such as a tissue section or cell sample. The sample is contacted with an antibody that binds to the target antigen of interest. The antibody is then detected using any of a variety of techniques. In some embodiments, the antibody that binds to the antigen (primary antibody) or a secondary antibody that binds to the primary antibody has been tagged or conjugated with a detectable label. In some embodiments a label-free detection method is used. A detectable label may be, for example, a fluorescent dye (e.g., a fluorescent small molecule) or quencher, colloidal metal, quantum dot, hapten, radioactive atom or isotope, or enzyme (e.g., peroxidase). It will be appreciated that a detectable label may be directly detectable or indirectly detectable. For example, a fluorescent dye would be directly detectable, whereas an enzyme may be indirectly detectable, e.g., the enzyme reacts with a substrate to generate a directly detectable signal. Numerous detectable labels and strategies that may be used for detection, e.g., immunological detection, are known in the art. Exemplary immunological detection methods include, e.g., immunohistochemistry (IHC); enzyme-linked immunosorbent assay (ELISA), bead-based assays such as the Luminex® assay platform (Invitrogen), flow cytometry, protein microarrays, surface plasmon resonance assays (e.g., using BiaCore technology), microcantilevers, immunoprecipitation, immunoblot (Western blot), etc. IHC generally refers to immunological detection of an antigen of interest (e.g., a cellular constituent) in a tissue sample such as a tissue section. As used herein, IHC is considered to encompass immunocytochemistry (ICC), which term generally refers to the immunological detection of a cellular constituent in isolated cells that essentially lack extracellular matrix components and tissue microarchitecture that would typically be present in a tissue sample. Traditional ELISA assays typically involve use of primary or secondary antibodies that are linked to an enzyme, which acts on a substrate to produce a detectable signal (e.g., production of a colored product) to indicate the presence of antigen or other analyte. IHC generally refers to the immunological detection of a tissue or cellular constituent in a tissue or cell sample comprising substantially intact (optionally permeabilized) cells. As used herein, the term “ELISA” also encompasses use of non-enzymatic reporters such as fluorogenic, electrochemiluminescent, or real-time PCR reporters that generate quantifiable signals. It will be appreciated that the term “ELISA” encompasses a number of variations such as “indirect”, “sandwich”, “competitive”, and “reverse” ELISA.

In some embodiments, e.g., wherein IHC is used for detecting HSF1, a sample is in the form of a tissue section, which may be a fixed or a fresh (e.g., fresh frozen) tissue section or cell smear in various embodiments. A sample, e.g., a tissue section, may be embedded, e.g., in paraffin or a synthetic resin or combination thereof. A sample, e.g., a tissue section, may be fixed using a suitable fixative such as a formalin-based fixative. The section may be a paraffin-embedded, formalin-fixed tissue section. A section may be deparaffinized (a process in which paraffin (or other substance in which the tissue section has been embedded) is removed (at least sufficiently to allow staining of a portion of the tissue section). To facilitate the immunological reaction of antibodies with antigens in fixed tissue or cells it may be helpful to unmask or “retrieve” the antigens through pretreatment of the sample. A variety of antigen retrieval procedures (sometimes called antigen recovery), can be used in IHC. Such methods can include, for example, applying heat (optionally with pressure) and/or treating with various proteolytic enzymes. Methods can include microwave oven irradiation, combined microwave oven irradiation and proteolytic enzyme digestion, pressure cooker heating, autoclave heating, water bath heating, steamer heating, high temperature incubator, etc. To reduce background staining in IHC, the sample may be incubated with a buffer that blocks the reactive sites to which the primary or secondary antibodies may otherwise bind. Common blocking buffers include, e.g., normal serum, non-fat dry milk, bovine serum albumin (BSA), or gelatin, and various commercial blocking buffers. The sample is then contacted with an antibody that specifically binds to the antigen whose detection is desired (e.g., HSF1 protein). After an appropriate period of time, unbound antibody is then removed (e.g., by washing) and antibody that remains bound to the sample is detected. After immunohistochemical staining, a second stain may be applied, e.g., to provide contrast that helps the primary stain stand out. Such a stain may be referred to as a “counterstain”. Such stains may show specificity for discrete cellular compartments or antigens or stain the whole cell. Examples of commonly used counterstains include, e.g., hematoxylin, Hoechst stain, or DAPI. The tissue section can be visualized using appropriate microscopy, e.g., light microscopy, fluorescence microscopy, etc. In some embodiments, automated imaging system with appropriate software to perform automated image analysis is used.

In some embodiments, flow cytometry (optionally including cell sorting) is used to detect HSF1 expression. The use of flow cytometry would typically require the use of isolated cells substantially removed from the surrounding tissue microarchitecture, e.g., as a single cell suspension. HSF1 mRNA or polypeptide level could be assessed by contacting cells with a labeled probe that binds to HSF1 mRNA or a labeled antibody that binds to HSF1 protein, respectively, wherein said probe or antibody is appropriately labeled (e.g., with a fluorophore, quantum dot, or isotope) so as to be detectable by flow cytometry. In some embodiments, cell imaging can be used to detect HSF1.

In some embodiments, an antibody for use in an immunological detection method, e.g., IHC, is monoclonal. In some embodiments an antibody is polyclonal. In some embodiments, an antibody is a preparation that comprises multiple monoclonal antibodies. In some embodiments, the monoclonal or polyclonal antibodies have been generated using the same portion of HSF1 (or full length HSF) as an immunogen or binding target. In some embodiments, an antibody is an anti-peptide antibody. In some embodiments, a monoclonal antibody preparation may comprise multiple distinct monoclonal antibodies generated using different portions of HSF1 as immunogens or binding targets. Many antibodies that specifically bind to HSF1 are commercially available and may be used in embodiments of the present invention. One of ordinary skill in the art would readily be able to generate additional antibodies suitable for use to detect HSF1 polypeptide using standard methods.

In some embodiments, a ligand that specifically binds to HSF1 but is not an antibody is used as an affinity reagent for detection of HSF1. For example, nucleic acid aptamers or certain non-naturally occurring polypeptides structurally unrelated to antibodies based on various protein scaffolds may be used as affinity reagents. Examples include, e.g., agents referred to in the art as affibodies, anticalins, adnectins, synbodies, etc. See, e.g., Gebauer, M. and Skerra, A., Current Opinion in Chemical Biology, (2009), 13(3): 245-255 or PCT/US2009/041570. In some embodiments an aptamer is used as an affinity reagent. The terms “affinity reagent” and “binding agent” are used interchangeably herein.

In some embodiments, a non-affinity based method is used to assess the level of HSF1 polypeptide or HSF1 activation. For example, mass spectrometry could be used to detect HSF1 or to specifically detect phosphorylated HSF1.

In some embodiments, an antibody (or other affinity reagent) or procedure for use to detect HSF1 (or HSF1 phosphorylated on serine 326) can be validated, if desired, by showing that the classification obtained using the antibody or procedure correlate with a phenotypic characteristic of interest such as presence or absence of CIS, cancer prognosis, or treatment outcome, in an appropriate set of samples. For example, as described in the Examples, a commercially available monoclonal antibody preparation RT-629-PABX (Thermo Scientific) comprising a combination of rat monoclonal antibodies (“antibody cocktail”) was validated for use in IHC for detection of HSF1 and classification of samples and subjects into different categories correlated with presence or absence of CIS, cancer prognosis, or treatment outcome. Other exemplary antibodies of use for detecting or isolating HSF1 are also disclosed in the Examples. In some embodiments, an antibody or antibody preparation or a protocol or procedure for performing IHC may be validated for use in an inventive method by establishing that its use provides similar results to those obtained using RT-629-PABX and the procedures described in the Examples on an appropriate set of test samples. For example, an antibody or antibody preparation or a procedure may be validated by establishing that its use results in the same classification (concordant classification) of at least 80%, 85%, 90%, 95% or more of samples in an appropriate set of test samples as is obtained using the antibody preparation of RT-629-PABX. A set of test samples may be selected to include, e.g., at least 10, 20, 30, or more samples in each category in a classification scheme (e.g., “positive” and “negative” categories; categories of“no”, “low”, or “high” expression, scores of 1, 2, 3; etc.). In some embodiments, a set of test samples comprises breast tissue samples, e.g., from the NHS. In some embodiments a set of samples is in the form of a tissue microarray. Once a particular antibody or procedure is validated, it can be used to validate additional antibodies or procedures. Likewise, a probe, primer, microarray, or other reagent(s) or procedure(s) to detect HSF1 RNA can be validated, if desired, by showing that the classification obtained using the reagent or procedure correlates with a phenotypic characteristic of interest such as presence or absence of CIS, cancer prognosis, or treatment outcome, in an appropriate set of samples.

It will be understood that suitable controls and normalization procedures can be used to accurately quantify HSF1 expression, where appropriate. For example, measured values can be normalized based on the expression of one or more RNAs or polypeptides whose expression is not correlated with a phenotypic characteristic of interest. In some embodiments, a measured value can be normalized to account for the fact that different samples may contain different proportions of a cell type of interest, e.g., cancer cells, versus non-cancer cells. For example, in some embodiments, the percentage of stromal cells, e.g., fibroblasts, may be assessed by measuring expression of a stromal cell-specific marker, and the overall results adjusted to accurately reflect HSF1 mRNA or polypeptide level specifically in the tumor cells. Similarly, appropriate controls and normalization procedures can be used to accurately quantify HSF1 activation, where appropriate. It would also be understood that if a sample such a tissue section contains distinguishable (e.g., based on standard histopathological criteria), areas of neoplastic and non-neoplastic tissue, such as at the margin of a tumor, the level of HSF1 expression or activation could be assessed specifically in the area of neoplastic tissue, e.g., for purposes of comparison with a control level, which may optionally be the level measured in the non-neoplastic tissue.

In certain embodiments of the invention the level of HSF1 mRNA or protein level is not measured or analyzed simply as a contributor to a cluster analysis, dendrogram, or heatmap based on gene expression profiling in which expression at least 20; 50; 100; 500; 1,000, or more genes is assessed. In certain embodiments of the invention, e.g., if HSF1 mRNA or protein level is measured as part of such a gene expression profile, the level of HSF1 mRNA or protein is used to classify samples or tumors (e.g., for diagnostic, prognostic or treatment-specific predictive purposes) in a manner that is distinct from the manner in which the expression of many or most other genes in the gene expression profile are used. For example, the level of HSF1 mRNA or polypeptide may be used independently of most or all of the other measured expression levels or may be weighted more strongly than many or most other mRNAs in analyzing or using the results.

In some embodiments, HSF1 mRNA or polypeptide level is used together with levels of a set of no more than 10 other mRNAs or proteins that are selected for their utility for classification for diagnostic, prognostic, or predictive purposes in one or more types of cancer, such as breast cancer. For example, in the case of breast cancer, HSF1 mRNA or polypeptide levels can be used together with a measurement of estrogen receptor (ER), progesterone receptor (PR), or human epidermal growth factor receptor 2 (HER2) mRNA or polypeptide levels. In some embodiments, measurement of ER, PR, HER2 mRNA and/or other mRNA is performed using ISH. In some embodiments, measurement of ER, PR, HER2 polypeptide and/or other polypeptides is performed using IHC. In some embodiments such testing is performed in accordance with recommendations of the American Society of Clinical Oncology/College of American Pathologists Guideline Recommendations for Immunohistochemical Testing of Estrogen and Progesterone Receptors in Breast Cancer or the American Society of Clinical Oncology/College of American Pathologists Guideline Recommendations for Human Epidermal Growth Factor Receptor 2 Testing in Breast Cancer. In some embodiments such testing is performed according to recommendations of a commercially available kit, e.g., a kit approved by a governmental regulatory agency (e.g., the U.S. Food and Drug Administration) for use in clinical diagnostic, prognostic, or predictive purposes.

In general, the level of HSF1 activation can be assessed using any of a variety of methods in various embodiments of the invention. In some embodiments, the level of HSF1 activation is determined by detecting HSF1 polypeptide in cell nuclei, wherein nuclear localization of HSF1 polypeptide is indicative of HSF1 activation. HSF1 localization can be assessed, for example, using IHC, flow cytometry, FACS, etc. Alternately, or additionally, cell nuclei could be isolated and HSF1 polypeptide detected by immunoblot. In some embodiments, HSF1 nuclear localization could be assessed by staining for HSF1 protein, counterstaining with a dye that binds to a nuclear component such as DNA, and assessing co-localization of HSF1 and such nuclear component. Cell imaging can be used in some embodiments. It will be understood that “detecting” as used herein, can encompass applying a suitable detection procedure and obtaining a negative result, i.e., detecting a lack of expression or activation.

In some embodiments, the level of HSF1 activation is determined by determining the level of HSF1 phosphorylation, wherein HSF1 phosphorylation is indicative of HSF1 activation. In some embodiments, phosphorylation of HSF1 on serine 326 is determined as an indicator of HSF1 activation. Phosphorylation of HSF1 on serine 326 can be assessed, for example, using antibodies that bind specifically to HSF1 phosphorylated on serine 326. In some embodiments, a ratio of phosphorylated HSF1 to unphosphorylated HSF1 (on serine 326) is used as an indicator of HSF1 activation, with a higher ratio indicating more activation. Measurement of other post-translational modifications indicative of HSF1 activation could be used in various embodiments.

In some embodiments, the level of HSF1 activation is determined by measuring a gene expression profile of one or more genes whose expression is regulated by HSF1, wherein increased expression of a gene that is positively regulated by HSF1 or decreased expression of a gene that is negatively regulated by HSF1 is indicative of HSF1 activation. In many embodiments, the HSF1-regulated gene is not an HSP (e.g., HSP90) or, if HSP expression is measured, at least one additional HSF1-regulated gene other than an HSP is also measured. In some embodiments a gene expression profile measures expression of at least 5 HSF1-regulated genes, e.g., between 5 and about 1,000 HSF1-regulated genes. In some embodiments at least some of the genes are HSF1-CP genes. In some embodiments at least some of the HSF1-CP genes are HSF1-CSS genes. In some embodiments at least some of the HSF1-CP genes are HSF1-CaSig2 genes. In some embodiments at least some of the HSF1-CP genes are HSF1-CaSig3 genes. In some embodiments at least some of the HSF1-CP genes are refined HSF1-CSS genes. In some embodiments at least some of the HSF1-CP genes are Module 1, Module 2, Module 3, Module 4, or Module 5 genes. Of course the gene expression profile may in some embodiments also measure expression of one or more genes that are not regulated by HSF1. In some embodiments measurement of expression of one or more genes that are not regulated by HSF1 is used as a control or for normalization purposes. In some embodiments measurement of expression of one or more genes that are not regulated by HSF1 may be disregarded. In some embodiments no more than 1%, 5%, 10%, 20%, 30%, 40%, or 50%, of measurements are of genes that are not bound and/or regulated by HSF1. In some embodiments, determining whether HSF1 is activated comprises comparing a gene expression profile obtained from a sample of interest with gene expression profile(s) obtained from one or more samples in which HSF1 is activated or is not activated. If the gene expression profile obtained from the sample clusters with or resembles the gene expression profile obtained from sample(s) in which HSF1 is activated, the sample of interest can be classified as exhibiting HSF1 activation. On the other hand, if the gene expression profile obtained from the sample of interest clusters with or resembles the gene expression profile obtained from sample(s) in which HSF1 is not activated, the sample of interest can be classified as not exhibiting HSF1 activation. Methods for clustering samples are well known in the art or assigning a sample to one of multiple clusters are well known in the art and include, e.g., hierarchical clustering, k-means clustering, and variants of these approaches.

In some embodiments, the level of HSF1 activation is determined by measuring binding of HSF1 to the promoter of one or more HSF1-regulated genes, wherein binding of HSF1 to the promoter of an HSF1-regulated gene is indicative of HSF1 activation. In some embodiments, an HSF1-regulated gene is a gene whose expression level (e.g., as assessed based on mRNA or protein levels) is increased or decreased by at least a factor of 1.2 as a result of HSF1 activation. In some embodiments, an HSF1-regulated gene is among the 1,000 genes in the human genome whose expression is most strongly affected (increased or inhibited) by HSF1. In some embodiments, an HSF1-regulated gene is among the 1,000 genes in the human genome whose promoter is most strongly bound by HSF1 under conditions in which HSF1 is activated. Methods for measuring binding of a protein (e.g., HSF1) to DNA (e.g., genomic DNA) include, e.g., chromatin immunoprecipitation using an antibody to the protein followed by microarray hybridization to identify bound sequences, commonly referred to as ChIP-on-chip (see, e.g., U.S. Pat. Nos. 6,410,243; 7,470,507; 7,575,869); ChIP-Sequencing, which uses chromatin immunoprecipitation followed by high throughput sequencing to identify the bound DNA; and DamID (DNA adenine methyltransferase identification; see, e.g., Vogel M J, et al (2007). “Detection of in vivo protein-DNA interactions using DamID in mammalian cells”. Nat Protoc 2 (6): 1467-78).

In some embodiments, an assay to detect HSF1 expression or activation makes use of fluorescence resonance energy transfer (FRET).

In some embodiments, the level of an HSF1 gene product or the level of HSF1 activation is determined to be “increased” or “not increased” by comparison with a suitable control level or reference level. The terms “reference level” and “control level” may be used interchangeably herein. A suitable control level can be a level that represents a normal level of HSF1 gene product or HSF1 activation, e.g., a level of HSF1 gene product or HSF1 activation existing in cells or tissue in a non-diseased condition and in the substantial absence of stresses that activate the heat shock response. Thus any method that includes a step of (a) assessing (determining) the level of HSF1 gene expression or the level of HSF1 activation in a sample can comprise a step of(b) comparing the level of HSF1 gene expression or HSF1 activation with a control level of HSF1 gene expression or HSF1 activation, wherein if the level determined in (a) is greater than the control level, then the level determined in (a) is considered to be “increased” (or, if the level determined in (a) is not greater than the control level, then the level determined in (a) is considered to be “not increased”. For example, if a tumor has an increased level of HSF1 expression or HSF1 activation as compared to a control level, the tumor is classified as having a high risk of poor outcome, while if the tumor does not have a significantly increased level of HSF1 relative to a control level, the tumor is classified as having a low risk of poor outcome. A control level may be determined in a variety of ways. In some embodiments a control level is an absolute level. In some embodiments a control level is a relative level, such as the percentage of tumor cells exhibiting nuclear HSF1 staining or the percentage of tumor cells or tumor cell nuclei exhibiting intense staining for HSF1. A comparison can be performed in various ways. For example, in some embodiments one or more samples are obtained from a tumor, and one or more samples are obtained from nearby normal (non-tumor) tissue composed of similar cell types from the same patient. The relative level of HSF1 gene product or HSF1 activation in the tumor sample(s) versus the non-tumor sample(s) is determined. In some embodiments, if the relative level (ratio) of HSF1 gene product in the tumor samples versus the non-tumor sample(s) is greater than a predetermined value (indicating that cells of the tumor have increased HSF1), the tumor is classified as high risk. In some embodiments the predetermined value is, e.g., at least 1.5, 2, 2.5, 3, 5, 10, 20, or more. In some embodiments the predetermined value is between about 1.5 and about 10. A control level can be a historical measurement. For example, the data provided herein provide examples of levels of HSF1 expression and HSF1 activation in normal breast, cervix, colon, lung, pancreas, prostate, and meningeal tissue and tissue from breast, cervix, colon, lung, pancreas, prostate, and meningeal tumors, thereby providing examples of suitable control levels. It will be understood that in at least some embodiments a value may be semi-quantitative, qualitative or approximate. For example, visual inspection (e.g., using light microscopy) of a stained IHC sample can provide an assessment of the level of HSF1 expression or HSF1 activation without necessarily counting cells or nuclei or precisely quantifying the intensity of staining.

Various risk categories may be defined. For example, tumors may be classified as at low, intermediate, or high risk of poor outcome. A variety of statistical methods may be used to correlate the risk of poor outcome with the relative or absolute level of HSF1 expression or HSF1 activation.

For purposes of description herein it is assumed that the control or reference level represents normal levels of HSF1 expression or HSF1 activation present in non-cancer cells and tissues. However, it will be understood that a level of HSF1 expression or HSF1 activation characteristic of cancer (e.g., breast cancer) could be used as a reference or control level. In that case, the presence of HSF1 expression or HSF1 activation at a level comparable to, e.g., approximately the same, as or greater than the control level would be indicative of the presence of cancer, poor cancer prognosis, aggressive cancer phenotype, or to identify a subject who is a suitable candidate for treatment with a proteostasis modulator, while a decreased level of HSF1 expression or HSF1 activation as compared with the control level would be predictive of good cancer prognosis, less aggressive cancer phenotype or to identify a subject who may not be a suitable candidate for treatment with a proteostasis modulator, etc.

Methods have generally been stated herein mainly in terms of conclusions or predictions that can be made if increased HSF1 expression or increased HSF1 activation is present. Methods could equally well have been stated in terms of conclusions or predictions that can be made if increased HSF1 expression or increased HSF1 activation is not present. For example, if HSF1 expression is absent in a sample being assessed for the presence or absence of cancer, the sample would not be classified as cancer based on HSF1 expression. If HSF1 expression or HSF activation is absent or low in a sample from an invasive tumor, the tumor would be classified as having a good prognosis. If HSF1 expression or HSF activation is absent or low in a sample from an invasive tumor, the subject may not benefit from treatment with a proteostasis modulator.

Any of the methods of the invention may, in certain embodiments, comprise assigning a score to a sample (or to a tumor from which a sample was obtained) based on the level of HSF1 expression or HSF1 activation measured in the sample, e.g., based on the level of an HSF1 gene product or the level of HSF1 activation or a combination thereof.

In some embodiments a score is assigned based on assessing both HSF1 polypeptide level and HSF1 activation level. For example, a score can be assigned based on the number (e.g., percentage) of nuclei that are positive for HSF1 and the intensity of the staining in the positive nuclei. For example, a first score (e.g., between 0 and 5) can be assigned based on the percentage positive nuclei, and a second score (e.g., between 0 and 5) assigned based on staining intensity in the nuclei. In some embodiments, the two scores are added to obtain a composite score (e.g., ranging between 0 and 10). In some embodiments the two scores are multiplied to obtain a composite score (e.g., ranging between 0 and 25). The range can be divided into multiple (e.g., 2 to 5) smaller ranges, e.g., 0-9, 10-18, 19-25, and samples or tumors are assigned an overall HSF1 expression/activation score based on which subrange the composite score falls into. For example, 0-9 is low, 10-18 is intermediate, and 19-25 is high in some embodiments. A higher score indicates, for example, increased aggressiveness, increased likelihood of poor outcome, poor prognosis. Thus in some aspects, the invention provides a method of assigning a score to a sample comprising cells, the method comprising steps of: (a) assigning a first score to the sample based on the number or percentage of cell nuclei that are positive for HSF1 protein; (b) assigning a second score to the sample based on the level of HSF1 protein in cell nuclei; and (c) obtaining a composite score by combining the scores obtained in step (a) and step (b). In some embodiments, combining the scores comprises adding the scores. In some embodiments combining the scores comprises multiplying the scores. In some embodiments the method further comprises assigning the sample to an HSF1 expression/activation category based on the composite score. It will be understood that if the sample is a tissue sample that comprises areas of neoplastic tissue and areas of non-neoplastic tissue (e.g., as identified using standard histopathological criteria), the score(s) can be assigned based on assessing neoplastic tissue. The non-neoplastic tissue may be used as a control.

In some embodiments, a score is assigned using a scale of 0 to X, where 0 indicates that the sample is “negative” for HSF1 (e.g., no detectable HSF1 polypeptide in cell nuclei), and X is a number that represents strong (high intensity) staining in the majority of cell nuclei. X can be, e.g., 2, 3, 4, or 5 in various embodiments. In some embodiments, a score is assigned using a scale of 0, 1, or 2, where 0 indicates that the sample is negative for HSF1 (no detectable HSF1 polypeptide in cell nuclei), 1 is low level nuclear staining and 2 is strong (high intensity) staining in the majority of cell nuclei. A higher score indicates a less favorable prognosis than a lower score, e.g., more likely occurrence of metastasis, shorter disease free survival, lower likelihood of 5 year survival, lower likelihood of 10 year survival, or shorter average survival. A score can be obtained by evaluating one field or multiple fields in a cell or tissue sample. Multiple samples from a tumor may be evaluated in some embodiments. It will be understood that “no detectable HSF1” could mean that the level detected, if any, is not noticeably or not significantly different to background levels. It will be appreciated that a score can be represented using numbers or using any suitable set of symbols or words instead of, or in combination with numbers. For example, scores can be represented as 0, 1, 2; negative, positive; negative, low, high; −, +, ++, +++; 1+, 2+, 3+, etc.

In some embodiments, at least 20, 50, 100, 200, 300, 400, 500, 1000 cells, or more (e.g., tumor cells) are assessed to evaluate HSF1 expression or HSF activation in a sample or tumor, e.g., to assign a score to a sample or tumor. In some embodiments, samples or tumors that do not exhibit HSF1 polypeptide in nuclei, e.g., as assessed using IHC, may be considered negative for HSF1.

The number of categories in a useful scoring or classification system can be at least 2, e.g., between 2 and 10, although the number of categories may be greater than 10 in some embodiments. The scoring or classification system often is effective to divide a population of tumors or subjects into groups that differ in terms of an outcome such as local progression, local recurrence, discovery or progression of regional or distant metastasis, death from any cause, or death directly attributable to cancer. An outcome may be assessed over a given time period, e.g., 2 years, 5 years, 10 years, 15 years, or 20 years from a relevant date. The relevant date may be, e.g., the date of diagnosis or approximate date of diagnosis (e.g., within about 1 month of diagnosis) or a date after diagnosis, e.g., a date of initiating treatment. Methods and criteria for evaluating progression, response to treatment, existence of metastases, and other outcomes are known in the art and may include objective measurements (e.g., anatomical tumor burden) and criteria, clinical evaluation of symptoms), or combinations thereof. For example, 1, 2, or 3-dimensional imaging (e.g., using X-ray, CT scan, or MRI scan, etc.) and/or functional imaging may be used to detect or assess lesions (local or metastatic), e.g., to measure anatomical tumor burden, detect new lesions, etc. In some embodiments, a difference between groups is statistically significant as determined using an appropriate statistical test or analysis method, which can be selected by one of ordinary skill in the art. In many embodiments, a difference between groups would be considered clinically meaningful or clinically significant by one of ordinary skill in the art.

HSF1 Mediates a Distinct Malignancy-Enabling Transcriptional Program in Cancer

Previous work in mice revealed that HSF1 is co-opted by tumor cells to promote their survival, to the detriment of their hosts. The importance of HSF1 in supporting carcinogenesis has been demonstrated in model systems by the dramatically reduced susceptibility of Hsf1-knockout mice to tumor formation. This has been established for cancers driven by oncogenic RAS, tumor suppressor p53 mutations, and chemical carcinogens. In addition to its role in tumor formation in mice, HSF1 fosters the growth of human tumor cells in culture. Depleting HSF1 from established human cancer lines markedly reduces their proliferation and survival (Dai et al., 2007; Meng et al., 2010; Min et al., 2007; Santagata et al., 2012; Zhao et al., 2011). In mouse models, HSF1 enables adaptive changes in a diverse array of cellular processes, including signal transduction, glucose metabolism and protein translation (Dai et al., 2007; Khaleque et al., 2008; Lee et al., 2008; Zhao et al., 2011; Zhao et al., 2009). The commonly held view is that HSF1 exerts this broad influence in cancer simply by allowing cells to manage the imbalances in protein homeostasis that arise in malignancy. According to this view, the main impact of HSF1 on tumor biology occurs indirectly, through the actions of molecular chaperones like Hsp90 and Hsp70 on their client proteins (Jin et al., 2011; Solimini et al., 2007).

Described herein is the discovery that HSF1 has a broad range of direct gene regulating effects (e.g., transactivating or repressing effects) in cancer cells. By comparing cells with high and low malignant potential alongside their non-transformed counterparts, Applicants identified an HSF1-regulated transcriptional program specific to malignant cells and distinct from heat shock. In a genome-wide survey of HSF1 DNA binding, numerous genes whose regulatory regions were bound by HSF1 in a highly malignant tumor cell line under normal temperature conditions were identified. Similar HSF1 binding patterns were observed in multiple human cancer cell lines of various cancer types and in human tumor samples, thus demonstrating the presence of a dramatic basal level of HSF1 activation in cancer even in the absence of thermal stress. The term “thermal stress” is used interchangeably herein with “heat shock” and refers to exposing cells to elevated temperature (i.e., temperature above physiologically normal for such cells) for a sufficient period of time to detectably, e.g., robustly, induce the heat shock response. One of ordinary skill in the art will know of suitable protocols to heat shock cells, e.g., mammalian cells, without causing substantial, e.g., irreversible, cell damage or death. In some embodiments heat shock comprises exposing cells to a temperature of 42±0.5 degrees C., e.g., 42 degrees C., for about 1 hour or similar exposures to elevated temperatures (e.g., at or above 40 or 41 degrees C.) resulting in similar or at least approximately equivalent induction of the heat shock response. In some embodiments heat shock comprises exposing cells to a temperature of 43±0.5 degrees C. or 44±0.5 degrees C. for, e.g., between 30 and 60 minutes. In some embodiments cells are not “pre-conditioned” by prior exposure to elevated temperature within a relevant time period, e.g., within 24 hours prior to heat shock. In some embodiments cells are pre-conditioned by prior exposure to elevated temperature within a relevant time period, e.g., within 24 hours prior to heat shock. In some embodiments cells are allowed to recover for up to about 60 minutes, e.g., about 30 minutes, at normal (sub-heat shock) temperature, e.g., 37 degrees C., prior to isolation of RNA or DNA. In some embodiments assessment of the effect of heat shock on expression may occur after allowing an appropriate amount of time for translation of a transcript whose expression is induced by HSF1. In some embodiments cells are returned to normal temperature conditions for no more than 2, 3, 4, 6, or 8 hours prior to assessment of the effect of heat shock (or harvesting of cells, RNA, or DNA for subsequent assessment). Unless otherwise indicated or evident from the context, the term “heat shocked cells” or “cells subjected to heat shock” refers to heat shocked non-transformed cells. The terms “non-transformed”, “non-cancer”, “non-tumorigenic”, and “non-tumor” are used interchangeably herein to refer to cells that are not cancer cells or tissue that is not tumor tissue. In some aspects, non-cancer cells lack morphological characteristics typical of cancer cells and lack the ability to form tumors when introduced into an immunologically compatible host. In some embodiments a non-cancer cell is a primary cell. In some embodiments a non-cancer cell is an immortal cell. In some embodiments an immortal non-cancer cell expresses human teloinerase catalytic subunit (hTERT) or a non-human ortholog thereof. In some embodiments a non-cancer cell is a cell that has been immortalized by introducing a nucleic acid encoding human telomerase catalytic subunit (hTERT) or a non-human ortholog thereof into the cell or an ancestor of the cell. In some embodiments non-transformed cells used as control cells for comparison with transformed cells are of the same type or tissue of origin as transformed cells with which they are compared. In some embodiments non-transformed cells are immortalized cells derived from normal (non-cancer) tissue. It is generally assumed herein that, unless otherwise indicated, heat shocked cells and cancer cells are not deliberately subjected to other stresses known to activate the heat shock response. However, the present disclosure encompasses embodiments in which HSF1 activity in response to alternate stresses rather than heat shock is compared with HSF1 cancer-related activity as described herein in detail with respect to heat shock.

HSF1 was found to regulate a transcriptional program in cancer cells that is distinct from the HSF1 transcriptional program elicited by heat shock. Some genes are bound by HSF1 in cancer cells, e.g., malignant cancer cells, but are not detectably bound by HSF1 in non-transformed control cells subjected to heat shock. Some genes are bound by HSF1 both in cancer cells, e.g., malignant cancer cells, and in heat shock conditions. In the case of many genes that are bound in both cancer cells and in non-transformed cells subjected to heat shock, HSF1 binding was found to differ quantitatively, resulting in different effects on transcription in cancer cells as compared with non-transformed cells subjected to heat shock. In some aspects, the present disclosure provides the insight that the broad influence exerted by HSF1 in cancer is not limited to indirect effects occurring through the actions of molecular chaperones like Hsp90 and Hsp70 (whose transcription is induced by HSF1) on their client proteins. Instead HSF1 plays a direct role in rewiring the transcriptome and, thereby, the physiology of cancer cells. As described herein, Applicants defined a genome-wide transcriptional program that HSF1 coordinates in malignancy. This program differs fundamentally from that induced by thermal stress (although some genes are shared between the two programs). Its activation is common in a wide variety of human cancers and is shown herein to be strongly associated with metastasis and death in at least the three cancers responsible for ˜30% of all cancer-related deaths worldwide: those of the breast, colon and lung. Furthermore, the very broad range of tumors in which immunohistochemical evidence of HSF1 activation is observed confirms that it plays a pervasive role throughout tumor biology.

Surprisingly, the types of cellular processes that HSF1 regulates in cancer constitute a diverse array that extends far beyond protein folding. Some of these processes were previously known to be affected by the loss of HSF1 (Dai et al., 2007; Jin et al., 2011; Zhao et al., 2009). To explain such results, a common assumption has been that the effects of HSF1 loss are ultimately due to reduced chaperone activity and altered protein homeostasis (Jin et al., 2011; Meng et al., 2010; Solimini et al., 2007). Applicants find that, in addition to regulating chaperone proteins, HSF1 binds to, and directly regulates, genes underlying diverse cancer-related biological processes. Without wishing to be bound by any theory, the remarkable breadth of the HSF1 cancer program in humans may explains why HSF1 is such a powerful modifier of tumorigenesis in multiple animal models (Dai et al., 2007; Jin et al., 2011; Zhao et al., 2009) and why HSF1 was identified as one of only six potent metastasis-promoting genes in a genome-wide screen for enhancers of invasion by malignant melanoma cells (Scott et al., 2011). Not only is the repertoire of HSF1-regulated genes in cancer much more extensive than just heat-shock genes, but even the manner in which some of the classical heat-shock genes are regulated diverges between cancers and heat shock. For example, while HSP90AA1 (HSP90), HSPD1 (HSP60) and HSPA8 (HSC70) are activated by HSF1 in both situations, regulation of other HSP genes such as HSPA6 (HSP70B′), a pillar of the heat-shock response, differs dramatically in these two states. Following thermal stress, HSPA6 is typically the most highly induced of all mRNAs, yet, surprisingly in cancer, HSPA6 is only bound very weakly by HSF1. Its expression is not significantly changed following HSF1 depletion and its transcript level does not correlate with that of HSF1 in a meta-analysis of 12,000 gene expression experiments (described below). This observation has implications for efforts to better understand the regulation of HSF1 in cancer, and to identify modulators of HSF1 activity in cancer. In some aspects, the present disclosure provides reporters that are more likely to capture elements of HSF1 biology distinct to the malignant state, as compared with the heat shock response, than reporters controlled by the HSPA6 promoter (Boellmann and Thomas, 2010; Stanhill et al., 2006) or reporters controlled by other promoters that are weakly bound or not bound by HSF1 in cancer cells.

Multiple mechanisms may regulate HSF1 activity during the classic heat shock response. These include the release of HSF1 from its normal sequestration by chaperones when unfolded substrates compete for chaperone binding. In addition, HSF1 is also subject to extensive post-translational modifications including acetylation, sumoylation and numerous phosphorylations (Anckar and Sistonen, 2011). Some of these heat-shock regulatory mechanisms are likely to be shared by cancer cells. For instance, impaired protein homeostasis driven by the accumulation of mutant, misfolding-prone oncoproteins (Shimizu et al., 2006) aneuploidy (Tang et al., 2011) and the increased rate of translation in cancer could chronically stimulate HSF1 activation by releasing it from sequestration from chaperones (Anckar and Sistonen, 2011). The present disclosure provides the insight that dysregulation of signaling pathways in cancer may drive post-translational modifications to HSF1 in cancer cells. Some of these signaling pathways (such as those responsible for phosphorylation at serine 326) may also function to post-translationally modify HSF1 in heat-shocked cells, but others will likely be unique to cancer, and in some embodiments, at least some such pathways may be distinct in different cancers. Among the prominent pathways most frequently activated in cancer are the EGFR/HER2 axis (Zhao et al., 2009), the RAS/MAPK pathway (Stanhill et al., 2006), and the insulin/IGFI-like growth factor system (Chiang et al., 2012) have been reported to alter HSF1 activity. Additional modes of cancer-specific regulation may include the binding of co-regulators. As known in the art, HSF1 binds to DNA sequences termed heat shock elements (HSEs). As described herein, many genes in the HSF1 cancer program differ from those of the classic heat shock response in having a different number of HSE repeats and different co-regulator binding sites.

For purposes hereof, a gene characterized in that its regulatory region is detectably bound by HSF1 in at least some cancers or cancer cell lines even in the absence of thermal stress (e.g., at 37 degrees C.) may be referred to as an “HSF1 cancer program” (HSF1-CP) gene. In some embodiments the regulatory region of an HSF1-CP gene is more highly bound by HSF1 in at least some cancers or cancer cell lines as compared with non-transformed control cells subjected to heat shock. In some embodiments, the regulatory region is at least 1.5, 2, 3, 4, 5, 10, 20, or 50-fold more highly bound in cancer cells than in non-transformed heat shocked control cells. In some embodiments, the regulatory region is detectably bound in cancer cells and not detectably bound (i.e., not bound above background levels) on non-transformed heat shocked control cells. In some embodiments the regulatory region of an HSF1-CP gene is more highly bound by HSF1 in a diverse set of cancers or cancer cell lines as compared with non-transformed control cells subjected to heat shock. Certain HSF1-CP genes whose regulatory regions were found to be more highly bound by HSF1 in a highly malignant cell line, as compared with non-transformed control cells subjected to heat shock, are listed in Table T4A and may be referred to herein Group A genes. Certain HSF1-CP genes whose regulatory regions were found to be bound by HSF1 both in a highly malignant cell line (BPLER) and in either of the non-transformed control cells (BPE or HME) subjected to heat shock (but not in non-transformed control cells not subjected to heat shock) are listed in Table T4B and may be referred to herein Group B genes. In some aspects, the terms “strongly bound”, “highly bound”, and similar terms refer to the amount of binding, which may be assessed, e.g., using an appropriate method such as ChIP-on-chip or ChIP-Seq). One of ordinary skill in the art will be aware of suitable computer programs and methods for, e.g., detecting binding peaks, quantifying binding strength, representing results, etc. Exemplary methods of performing ChIP-Seq and analyzing results thereof are provided in the Examples. Other examples may be found in, e.g., Kim H A, et al., A short survey of computational analysis methods in analysing ChIP-seq data. Hum Genomics. 2011 January; 5(2):117-23 or Giannopoulou, E G and Elemento, O., An integrated ChIP-seq analysis platform with customizable workflows, BMC Bioinformatics 2011, 12:277. Gene names as recognized in the art are used in the Tables. As noted above, sequences, e.g., mRNA and polypeptide sequences, in the NCBI Reference Sequence (RefSeq) database may be used as representative gene product sequences for a gene of interest, e.g., the HSF1-CP genes. Genomic sequences of such genes are readily available. Chromosomal locations can be readily retrieved and aligned to a genome build e.g., at the UCSC Genome Browser web site (http://genome.ucsc.edu/). As will be appreciated by those of ordinary skill in the art, in those gene names (e.g., in the Tables) that begin with a “C” followed by a number and include the term “ORF” followed by a number, such as C10ORF4, the number following the C indicates a chromosome, and the number following ORF indicates the number of the open reading frame (e.g., open reading frame 4) on the chromosome of that number (e.g., chromosome 10).

In some embodiments an HSF1-CP gene is characterized in that it is strongly bound by HSF1 in cancer cells. Representative examples of strong and weak binding and of genes that are strongly bound or weakly bound are provided in the Examples and Figures hereof. Representative examples of genes that are bound more strongly in cancer cells than heat shocked cells, bound less strongly in cancer cells than heat shocked cells, or bound to about the same extent in cancer cells and heat shocked cells are provided in the Examples and Figures hereof. Any such genes may be used in a method disclosed herein and/or as a comparator to classify binding as strong or weak and/or to classify binding as stronger in cancer cells than heat shocked cells, weaker in cancer cells than heat shocked cells, or shared (bound at reasonably similar levels in both cancer cells and heat shocked cells) in various embodiments. In some embodiments, “weak binding” is binding at about the same level as HSF1 binds to HSPA6 in metastatic cancer cells such as BPLER cells. In some embodiments, “strong binding” is binding at about the same level as HSF1 binds to HSPA6 in non-transformed heat shocked control cells such as heat shocked BPE cells or binding at about the same level as HSF1 binds to HSPA8 in metastatic cancer cells such as BPLER cells. In some embodiments strong binding is binding at about the same level as HSF1 binds to CKS2, LY6K, or RBM23 in metastatic cancer cells such as BPLER cells. In some embodiments an HSF1-CP gene is among the 5%, 10%, 20%, 30%, 40%, or 50% genes that are most highly bound by HSF1 in cancer cells, e.g., in metastatic cancer cells such as BPLER cells.

In some embodiments a characteristic, property, or result is considered to be present “in cancer” or “in cancer cells” if it is evident in a specific cancer, cancer type, or cancer cell line. In some embodiments a characteristic, property, or result is considered to be present in “cancer” if it is evident in at least some members of a diverse set of cancers or cancer cell lines, e.g., at least 30%, 40%, 50%, 60%, 70%, 80%, 85%, 90%, or more of the members in a diverse set of cancers or cancer cell lines. In some embodiments a measurement representative of “cancer” may be obtained by obtaining an average of values measured in a diverse set of cancers or cancer cell lines. In some embodiments members of a diverse set of cancers or cancer cell lines are randomly selected, or at least not selected with knowledge of whether or not a particular characteristic, property, or result of interest is evident in the cancer or cancer cell line. In some embodiments a diverse set of cancers or cancer cell lines comprises at least 5, 10, 20, 25, 30, 40, 50, 100, 200, 500, or 1,000, or more cancers and/or cancer cell lines. In some embodiments at least some of such cancers and/or cancer cell lines are of different types. For example, in some embodiments a diverse set of cancers or cancer cell lines comprises at least 3, 5, 10, 20, or more cancer types. In some embodiments a diverse set of cancer cell lines includes between 1 and 15 of the following cancer cell lines: BT474, H441, H838, H1703, HCC38, HCC1954, HCT15, HT29, SKBR3, SW620, ZR75-1, BT20, MDA-MB-231, MCF7, T47D cells. In some embodiments a diverse set of cancer cell lines comprises the NCI-60 cancer cell lines, or a randomly selected subset thereof. If desired, cells may be tested to confirm whether they are derived from a single individual or a particular cell line by any of a variety of methods known in the art such as DNA fingerprinting (e.g., short tandem repeat (STR) analysis) or single nucleotide polymorphism (SNP) analysis (which may be performed using, e.g., SNP arrays (e.g., SNP chips) or sequencing), etc. If desired, a cell or cell line, e.g., a cancer cell or cancer cell line, or a tissue sample may be classified as being of a particular type or having a particular tissue of origin based at least in part on expression of characteristic cellular markers, e.g., cell surface markers. Such markers are known to those of ordinary skill in the art. In some embodiments a diverse set of cancer cell lines or cancers comprises solid tumors, e.g., carcinomas and/or sarcomas. In some embodiments a diverse set of cancer cell lines or cancers comprises at least one cancer cell line or cancer that one of ordinary skill in the art would consider representative of adenocarcinomas. In some embodiments a diverse set of cancer cell lines or cancers includes at least one cancer cell line or cancer that one of ordinary skill in the art would consider representative of breast, lung, and colon cancer cell lines or breast, lung, and colon cancers. A cancer or cancer cell line may be represented by a sample, e.g., in a tissue microarray, tissue or cell bank or repository, etc. In some embodiments a cancer or cancer cell line is represented by a dataset, e.g., in a publicly available database such as Oncomine (https://www.oncomine.org/resource/login.html), ArrayExpress (www.ebi.ac.uk/arrayexpress/), NCBI's Gene Expression Omnibus (www.ncbi.nlm.nih.gov/geo/), Celsius (Day, A., et al., Genome Biology 2007, 8:R112; http://celsius.genome.ucla.edu/), or published in the scientific literature. A dataset may comprise, e.g., gene expression information, such as microarray data or RNA-Seq data, DNA binding information such as ChIP-chip or ChIP-Seq data, etc. Exemplary non-transformed cell lines, which may be used as control cells, include, e.g., HME, BPE, and MCF10A. In some embodiments a cell line that has comparable characteristics with respect to heat shock response as such cells may be used. In some embodiments historical control data are used.

Numerous tumor cell lines and non-transformed cell lines, in addition to those exemplified or mentioned herein, are known in the art. Cell lines may be obtained, e.g., from depositories or cell banks such as the American Type Culture Collection (ATCC), Coriell Cell Repositories, Deutsche Sammlung von Mikroorganismen und Zellkulturen (German Collection of Microorganisms and Cell Cultures; DSMZ), European Collection of Cell Cultures (ECACC), Japanese Collection of Research Bioresources (JCRB), RIKEN, Cell Bank Australia, etc. The paper and online catalogs of the afore-mentioned depositories and cell banks are incorporated herein by reference. In some embodiments non-cancer cells, e.g., a non-transformed cell line, originates from normal tissue not showing evidence of cancer. In some embodiments non-cancer cells have not had exogenous genetic material introduced therein. In some embodiments tumor cells, e.g., a tumor cell line, originate from a human tumor. In some embodiments tumor cells, e.g., a tumor cell line, originates from a tumor of a non-human animal, e.g., a tumor that was not produced by introduction of tumor cells into the non-human animal. In some embodiments tumor cells originate from a naturally arising tumor (i.e., a tumor that was not intentionally induced or generated for, e.g., experimental purposes). In some embodiments a cancer cell line or cancer is metastatic. A metastatic cancer cell line may be derived from a metastatic cancer and/or may have been shown to be capable of producing metastases in a non-human animal into which the cells have been introduced. In some embodiments a cancer cell line is highly tumorigenic. For example, the cancer cell line may be capable of giving rise to a tumor upon injection of, on average, between about 100-1,000 cells into an appropriate non-human animal host. In some embodiments experimentally produced tumor cells may be used. In some embodiments an experimentally produced tumor cell may be produced by genetically modifying a non-transformed cell. In some embodiments an engineered tumor cell may be produced from a non-tumor cell by a method that comprises expressing or activating an oncogene in the non-tumor cell and/or inactivating or inhibiting expression of one or more tumor suppressor genes or inhibiting activity of a gene product of a tumor suppressor gene. One of ordinary skill in the art will be aware of numerous oncogenes and tumor suppressor genes and methods of expressing or inhibiting expression thereof. Certain experimentally produced tumor cells and exemplary methods of producing tumor cells are described in PCT/US2000/015008 (WO/2000/073420) and/or in U.S. Ser. No. 10/767,018. In certain embodiments a non-tumor cell may be immortalized by a method comprising causing the cell to express telomerase catalytic subunit (e.g., human telomerase catalytic subunit; hTERT), to produce a non-transformed cell line. In some embodiments a tumor cell may be produced from a non-tumor cell by a method that comprises genetically modifying the non-tumor cell, e.g., by introducing one or more expression vector(s) comprising an oncogene into the cell or modifying an endogenous gene (proto-oncogene or tumor suppressor gene) by a targeted insertion into or near the gene or by deletion or replacement of a portion of the gene. In some embodiments the engineered tumor cell ectopically expresses hTERT, SV40-Large T Ag (LT) and H-Ras (RAS).

In some embodiments an HSF1-CP gene is characterized in that its expression in cancer cells increases or decreases by at least a factor of 1.2, 1.5, 2.0, 2.5, 3.0, 4.0, 5.0, or more following inhibition of HSF1 expression by, e.g., RNA interference. In some embodiments inhibition of HSF1 expression is by at least 25%, 50%, 60%, 70%, 80%, 90%, or more. In some embodiments expression of an HSF1-CP gene by cells in which HSF1 expression is inhibited is measured under conditions in which such inhibition does not result in substantial loss of cell viability (e.g., at a time point before maximum reduction in HSF1 level).

In some aspects, the invention relates to a set of 456 HSF1-CP genes characterized in that their promoter regions were found to be bound by HSF1 across a diverse set of malignant cell lines (see Examples). For purposes hereof such genes may be referred to as an “HSF1 cancer signature set” (sometimes abbreviated herein as HSF1-CSS or HSF1-CaSig) (Table T4C). As described further below, increased average expression of the HSF1-CSS genes was shown to correlate with decreased survival in a variety of representative human cancer types. In some aspects, the invention provides methods of assessing expression of one or more HSF-CSS genes, reagents useful for assessing expression of one or more HSF-CSS genes, and methods of using results of such assessment. In some aspects, subsets of the HSF1-CP genes or HSF1-CSS genes, reagents useful for modulating expression of such subsets, reagents useful for assessing or expression of such subsets, and methods of using results of such assessment, are provided. As used herein, a set C is considered a “subset” of a set D, if all elements (members) of C are also elements of D, but C is not equal to D (i.e. there exists at least one element of D not contained in C). Thus, a subset of the HSF1-CSS includes between 1 and 455 genes of the HSF1-CSS. Any and all such subsets are provided. In some embodiments a subset has between 300 and 400 genes. In some embodiments a subset has between 200 and 300 genes. In some embodiments a subset has between 100 and 200 genes. In some embodiments a subset has between 50 and 100 genes. In some embodiments a subset has between 25 and 50 genes. In some embodiments a subset has between 10 and 25 genes. In some embodiments a subset has between 5 and 10 genes. A subset of the HSF1-CSS genes may be referred to as a “refined HSF1-CSS”. In some aspects, a refined HSF1-CSS is useful for at least some of the same purposes as the full HSF1-CSS. For example, in some embodiments increased average expression of a refined HSF1-CSS correlates with decreased survival. In some embodiments, increased average expression of a refined HSF1-CSS correlates with decreased survival approximately equally well or at least as well as increased average expression of the HSF1-CSS. In some embodiments a refined HSF1-CSS has between 200 and 350 genes. In some embodiments a refined HSF1-CSS has between 100 and 200 genes, e.g., about 150 genes. An exemplary refined HSF1-CSS having 150 genes is presented in Table T4D. In some embodiments a refined HSF1-CSS has between 50 and 100 genes. In some embodiments a refined HSF1-CSS has between 25 and 50 genes. In some embodiments a refined HSF1-CSS has between 10 and 25 genes. In some embodiments a refined HSF1-CSS has between 5 and 10 genes. In some embodiments a subset of the HSF1-CP genes comprises the genes listed in Table T4G, T4H, or T4I.

In some aspects, the invention relates to additional HSF1 cancer signature sets composed of subsets of genes in the HSF1-CP. In some embodiments, a subset of the HSF1-CP genes is composed of HSF1-Module 1 and Module 2 genes. A representative subset of the HSF1-CP genes, which subset is composed of Module 1 and Module 2 genes is presented in Table T4E (this HSF1 cancer signature set is also referred to herein as “HSF1-CaSig2”). Genes in the HSF1-CaSig2 were positively regulated by HSF1 in malignant cells. In some embodiments, a subset of the HSF1-CP genes contains both positively and negatively regulated genes. An exemplary embodiment of such a subset is presented in Table T4F (this HSF1 cancer signature set is also referred to herein as “HSF1-CaSig3”). As described in further detail in the Examples, HSF1-CaSig, HSF1-CaSig2, and HSF1-CaSig3 signatures were strongly associated with patient outcome across multiple tumor types. In aspect herein in which the HSF-CSS genes are used, embodiments are provided in which the HSF-CaSig2 genes (listed in Table T4E) are used unless otherwise indicated or evident from the context. In aspect herein in which the HSF-CSS genes are used, embodiments are provided in which the HSF-CaSig3 genes (listed in Table T4F) are used unless otherwise indicated or evident from the context.

In some embodiments, an HSF1-CSS or refined HSF1-CSS disclosed herein may be further refined. In some embodiments, refinement may be performed by omitting one or more genes from the HSF1-CSS or refined HSF1-CSS to produce a reduced set of genes. The ability of the reduced set of genes to predict patient outcome across multiple datasets representing one or more tumor types can be determined. In some embodiments, a reduced set of genes is at least as effective as the HSF-CaSig, HSF1-CaSig2, or HSF1-CaSig3 genes in predicting patient outcome.

In some embodiments the invention relates to additional HSF1-CSS genes selected from among the HSF1-CP genes. In some embodiments an additional HSF1-CSS may be selected by identifying a subset of HSF1-CP genes composed of at least some HSF1-CP genes that are most positively correlated with poor outcome or composed of at least some HSF1-CP genes that most negatively correlated (anti-correlated) with poor outcome (based on a suitable statistic such as a t-test statistic) in one or more datasets containing tumor gene expression data. In some embodiments an additional HSF1-CSS may be selected by identifying a subset of HSF1-CP genes composed of (i) at least some HSF1-CP genes that are most positively correlated with poor outcome (ii) at least some HSF1-CP genes that most negatively correlated with poor outcome in one or more datasets containing tumor gene expression data. The number of positively and negatively correlated genes may be the same or different. In some embodiments, genes present in the relevant group (i.e., positively correlated with poor outcome or negatively correlated with poor outcome) in at least 50%, 60%, 70%, 80%, 90%, or more of the datasets are used in the additional HSF1-CSS. In some embodiments the ability of an additional HSF1-CSS to predict patient outcome may be validated using one or more tumor gene expression datasets not used for selection of such HSF1-CSS.

In some embodiments, tumor gene expression data that are used to select an additional HSF1-CSS is composed largely (e.g., at least 80%, 90%, 95%) or entirely of data obtained from tumors of a particular tumor type, subtype, or tissue of origin and/or excludes tumors of a particular tumor type, subtype or tissue of origin. Tumors of any tumor type, subtype or tissue of origin may be included or excluded. In some embodiments a tumor subtype is at least in part defined based on expression of one or more markers, molecular features, histopathological features, and/or clinical features, used in the art for tumor classification or staging. For example, in the case of breast cancer, a subtype may be defined based at least in part on expression of ER, PR, HER2/neu, and/or EGFR and/or on lymph node status. In some embodiments, an HSF1 cancer signature set selected using expression data from tumors of one or more selected tumor types, subtypes, or tissues of origin is of particular use for classifying or providing prognostic, diagnostic, predictive, or treatment selection information with regard to tumors of such selected tumor types, subtypes, or tissues of origin, e.g., the CSS may perform particularly well with regard to such tumors as compared with its performance among tumors of other types, subtypes, or tissues of origin. In some embodiments, the CSS is of use for classifying or providing prognostic, diagnostic, predictive, or treatment selection information with regard to tumors of other tumor types, subtypes, or tissues in addition to tumors of the selected type, subtype, or tissue of origin. For example, as described herein, HSF1 cancer signature sets derived from breast tumor expression data are useful in the context of lung and colon tumors, as well as breast tumors. In some embodiments, an HSF1 cancer signature set is selected using expression data from tumors of multiple different tumor types, subtypes, or tissues of origin. In some embodiments such an HSF1 cancer signature set of use in classifying or providing prognostic, diagnostic, predictive, or treatment selection information with regard to tumors of any of multiple selected tumor types, subtypes, or tissues of origin which may include, but not be limited to, tumors of the types, subtypes, or tissues of origin from which the expression data used to obtain the signature was obtained.

Further provided are sets of genes that comprise (a) (i) the HSF1-CSS or (ii) at least one subset of the HSF1-CSS (but not the full HSF1-CSS); and (b) at least one additional gene that is not within the HSF1-CSS. In some embodiments one or more additional gene(s) may be useful for any one or more purposes for which the HSF1-CSS is of use. In some embodiments one or more additional gene(s) may be useful as controls or for normalization.

In some embodiments, a subset of the HSF1-CP comprises or consists of genes that are coordinately regulated in cancer cells. In some embodiments a group of coordinately regulated genes may be referred to as a “module”. In some embodiments coordinately regulated genes are characterized in that their mRNA expression levels correlate across a set of diverse cancer cell lines or cancer samples. In some embodiments the Pearson correlation coefficient of the mRNA expression levels of coordinately regulated genes is at least 0.5, 0.6, or 0.7 across diverse cancer cell lines or cancer samples. In some embodiments coordinately regulated genes are characterized in that their expression level (e.g., as assessed by mRNA level) in cancer cells increases or decreases in the same direction following inhibition of HSF1 expression. In some embodiments, an HSF1-CP module comprises genes involved in protein folding, translation and/or mitosis (Module 1). In some embodiments, an HSF1-CP module comprises RNA binding genes and/or DNA damage binding genes (Module 2). In some aspects, transcription of genes in Module 1 or 2 is positively regulated (activated) by HSF1. In some embodiments, an HSF1-CP module comprises genes involved in immune functions or death receptor signaling (Module 3), insulin secretion (Module 4), or apoptosis, development, or insulin secretion (Module 5). In some aspects, transcription of genes in Module 3, 4, or 5 is negatively regulated (repressed) by HSF1. In some embodiments, modules are based at least in part on datasets that comprise data obtained using multiple probes for at least some genes. In some embodiments, a module is refined by excluding genes for which fewer than 50%, 60%, 70%, 80%, 90%, or more (e.g., 100%) of the probes fall within the module.

In some embodiments a subset of the HSF1-CP genes comprises or consists of genes that are involved in a process, pathway, or structure of interest or have a biological function or activity of interest. In some embodiments a gene may be classified as being involved in a process, pathway, or structure or as having a particular biological function or activity based on annotation in an art-recognized database such as the Gene Ontology database (http://www.geneontology.org/), KEGG database (http://www.genome.jp/kegg/), or Molecular Signatures database (http://www.broadinstitute.org/gsea/msigdb/index.jsp). In some embodiments a subset of the HSF1-CP comprises or consists of genes that are involved in protein folding, stress response, cell cycle, signaling, DNA repair, chromatin remodeling (e.g., chromatin modifying enzymes), apoptosis, transcription, mRNA processing, translation, energy metabolism, adhesion, development, and/or extracellular matrix. In some embodiments a subset of the HSF1-CP comprises or consists of genes that are involved in any of two or more processes, pathways, or structures of interest.

Wherever an aspect or embodiment disclosed herein refers to the HSF1-CP genes and/or HSF1-CSS genes, aspects or embodiments pertaining to each of(l) Group A, (2) Group B, (3) refined HSF1-CSS, (4) Module 1, (5) Module 2, (6) Module 3, (7) Module 4, (8) Module 5, (9) HSF1-CaSig2, (10) HSF1-CaSig3, and (12) subsets of any of the foregoing composed of genes that are more highly bound in cancer cells than in heat shocked, non-transformed control cells, are also disclosed herein, unless otherwise indicated or clearly evident from the context. For purposes of brevity, these individual aspects or embodiments may not always be expressly listed. It will be understood that certain details of such aspects or embodiments may differ depending, e.g., on whether the particular genes in the subset are positively or negatively regulated by HSF1 or positively or negatively correlated with poor (or good) outcome, treatment response, etc. In some aspects, measuring the expression of genes in the HSF1 cancer program is of use to classify cancers, to provide diagnostic or prognostic information. For example, high average expression of a set of genes whose promoter regions are bound by HSF1 in cancer cells (referred to herein as HSF1 cancer signature set (HSF1-CSS) genes) had a remarkable correlation with poor prognosis among multiple cohorts of breast cancer patients. The HSF1-CSS was more significantly associated with outcome than various well established prognostic indicators including the oncogene MYC, the proliferation marker Ki67 and MammaPrint, an expression-based diagnostic tool used in routine clinical practice (Kim and Paik, 2010). Expression of the HSF1-CSS was more strongly associated with poor outcome than any individual HSP transcript or even a panel of HSP genes. The HSF1-CSS was significantly associated with metastatic recurrence in women initially diagnosed with ER⁺/lymph node negative tumors. Increased expression of the HSF1-CSS in colon and lung cancers was strongly associated with reduced survival and more significantly associated with outcome than any individual HSP transcript or a panel of HSP genes.

In some embodiments, a method of diagnosing cancer in a subject comprises the steps of: determining the level of HSF1-CSS expression in a sample obtained from the subject, wherein increased HSF1-CSS expression in the sample is indicative that the subject has cancer. In some aspects, a method of identifying cancer comprises the steps of: (a) providing a biological sample; and (b) determining the level of HSF1-CSS expression in the sample, wherein increased HSF-CSS expression in the sample is indicative of cancer. In some embodiments a method of diagnosing or identifying cancer comprises comparing the level of HSF1-CSS expression with a control level of HSF1-CSS expression wherein a greater level in the sample as compared with the control level is indicative that the subject has cancer. In some embodiments, a method of assessing a tumor with respect to aggressiveness comprises: determining the level of HSF1-CSS expression in a sample obtained from the tumor, wherein an increased level of HSF1-CSS expression is correlated with increased aggressiveness, thereby classifying the tumor with respect to aggressiveness. In some embodiments the method comprises: (a) determining the level of HSF1-CSS expression in a sample obtained from the tumor; (b) comparing the level of HSF1-CSS expression with a control level of HSF1-CSS expression; and (c) assessing the aggressiveness of the tumor based at least in part on the result of step (b), wherein a greater level of HSF1-CSS expression in the sample obtained from the tumor as compared with the control level of is indicative of increased aggressiveness. In some embodiments, a method of classifying a tumor according to predicted outcome comprising steps of: determining the level of HSF1-CSS expression in a sample obtained from the tumor, wherein an increased level of HSF1-CSS expression is correlated with poor outcome, thereby classifying the tumor with respect to predicted outcome. In some embodiments the method comprises: (a) determining the level of HSF1-CSS expression in a tumor sample; and (b) comparing the level of HSF1-CSS expression with a control level of HSF1-CSS expression, wherein if the level determined in (a) is greater than the control level, the tumor is classified as having an increased likelihood of resulting in a poor outcome. In some embodiments a method of predicting cancer outcome in a subject comprises: determining the level of HSF1-CSS expression in a tumor sample from the subject, wherein an increased level of HSF1-CSS expression is correlated with poor outcome, thereby providing a prediction of cancer outcome. In some embodiments the method comprises (a) determining the level of HSF1-CSS expression in the tumor sample; and (b) comparing the level of HSF1-CSS expression with a control level of HSF1-CSS expression, wherein if the level determined in (a) is greater than the control level, the subject has increased likelihood of having a poor outcome. In some embodiments a method for providing prognostic information relating to a tumor comprises: determining the level of HSF1-CSS expression in a tumor sample from a subject in need of tumor prognosis, wherein if the level of HSF1-CSS expression is increased, the subject is considered to have a poor prognosis. In some embodiments the method comprises steps of: (a) determining the level of HSF1-CSS expression in the sample; and (b) comparing the level with a control level, wherein if the level determined in (a) is greater than the control level, the subject is considered to have a poor prognosis. In some embodiments a method for providing treatment-specific predictive information relating to a tumor comprises: determining the level of HSF1-CSS expression in a tumor sample from a subject in need of treatment-specific predictive information for a tumor, wherein the level of HSF1-CSS expression correlates with tumor sensitivity or resistance to a treatment, thereby providing treatment-specific predictive information. In some embodiments a method for tumor diagnosis, prognosis, treatment-specific prediction, or treatment selection comprises: (a) providing a sample obtained from a subject in need of diagnosis, prognosis, treatment-specific prediction, or treatment selection for a tumor; (b) determining the level of HSF1-CSS expression in the sample; (c) scoring the sample based on the level of HSF1-CSS expression, wherein the score provides diagnostic, prognostic, treatment-specific predictive, or treatment selection information. In some embodiments a control level of HSF1-CSS expression is a level representative of non-tumor tissue. In some embodiments, e.g., in a method for providing prognostic information, assessing tumor aggressiveness, or predicting cancer outcome, a control level of HSF1-CSS expression may be a level representative of tumors that have a good prognosis, low aggressiveness, or low propensity to metastasize or recur. In general, any method known in the art can be used to measure HSF1-CSS expression. For example, microarray analysis, nanostring technology, RNA-Seq, or RT-PCR may be used. In some embodiments a value representing an average expression level representative of the HSF1-CSS is obtained. Expression of an HSF1-CSS gene may be normalized, e.g., using a gene whose expression is not expected to change significantly in cancer versus non-transformed cells. In some embodiments actin is used for normalization. In some embodiments a method comprises classifying a tumor or tumor sample by comparing HSF1-CSS expression in the tumor or tumor sample with HSF1-CSS expression among a representative cohort of tumors that have known outcomes. In some embodiments clustering may be used to position a tumor sample of interest with respect to tumors having known outcomes. In some embodiments, tumors classified among the upper 25% of tumors by average expression level are determined to have a worse prognosis than tumors classified in the lower 75% (or any lower percentile, such as the lower 60%, 50%, 40%, 30%, etc.) In some embodiments a refined HSF1-CSS is used to classify tumors. In some embodiments expression of Module 1 or Module 2 genes is used to classify tumors. In some embodiments a refined HSF1-CSS is listed in Table T4D. In some embodiments HSF1-CaSig2 (Table T4E), or HSF1-CaSig3 (Table T4F) is used to classify tumors.

Without wishing to be bound by any theory, it is likely that the HSF1 cancer program supports the malignant state in a diverse spectrum of cancers because it regulates core processes rooted in fundamental tumor biology that ultimately affect outcome. The broad range of cancer types in which HSF1 is activated suggests that this program may have originated to support basic biological processes. Indeed, the sole heat-shock factor in yeast (yHSF), even at basal temperatures, binds many genes that are involved in a wide-range of core cellular functions (Hahn et al., 2004). These transcriptional targets allow yeast not only to adapt to environmental contingencies but also to modulate metabolism and maintain proliferation under normal growth conditions (Hahn et al., 2004; Hahn and Thiele, 2004). As a result, yHSF is essential for viability, paralleling the importance of HSF1 for the survival of cancer cells (Dai et al., 2007). Activation of HSF1 may also be advantageous in animals in states of high proliferation and altered metabolism such as immune activation and wound healing (Rokavec et al., 2012; Xiao et al., 1999; Zhou et al., 2008). Moreover, in diverse eukaryotes, HSF acts as a longevity factor. However, the evolutionarily ancient role played by HSF1 in helping cells to adapt, survive and proliferate is co-opted frequently to support highly malignant cancers. By enabling oncogenesis, the activation of this ancient pro-survival mechanism thereby actually impairs survival of the host. Without wishing to be bound by any theory, HSF1 activation in a particular tumor may reflect the degree to which accumulated oncogenic mutations have disrupted normal physiology even before overt invasion or metastasis occurs. This interpretation could explain the broad prognostic value of the HSF1-cancer signature across disparate cancers and even at early stages of disease. In some embodiments, the HSF1-CSS finds use as a sensitive measure of the malignant state and prognostic indicator. For example, in some embodiments the HSF1-CSS is of use in identifying tumors that are indolent and do not require intervention (e.g., wherein the tumor would not be expected to invade, metastasize, or progress to a state in which it impairs the functioning or physical condition of a subject or reduces the life expectancy of the subject), reducing the burdens of unnecessary treatment. In some embodiments the HSF1-CSS is of use in providing prognostic information or assessment of aggressiveness for a tumor of unknown tissue type or origin.

In some embodiments, an HSF1 cancer signature set or subset thereof is used to analyze one or more datasets (e.g., publicly available datasets) containing tumor gene expression data, wherein the dataset contains, in addition to gene expression data from tumors, information regarding an outcome or event of interest or one or more tumor characteristics associated with the corresponding tumor or subject having the tumor. In some embodiments, the HSF1 cancer signature set or subset thereof is used to classify tumors based on the expression data (e.g., into groups with high or low expression of the HSF1 cancer signature set or subset thereof). In some aspects, an HSF1 cancer signature set or subset thereof is used to identify or confirm a correlation between HSF1 activity and an outcome or event of interest in cancer (e.g., a poor outcome, good outcome, development of metastasis, survival, response (or lack of response) to a particular treatment, etc.) or one or more tumor characteristics. The predictive power of HSF1 activity with regard to an outcome of interest in cancer or one or more tumor characteristics may thus be identified or confirmed using an HSF1 cancer signature set or subset thereof as an indicator of HSF1 activity. In some aspects, the use of an HSF1 cancer signature set or subset thereof as a surrogate for HSF1 cancer-related activity leverages the availability of tumor gene expression datasets to identify or confirm a correlation between HSF1 activity and an outcome of interest in cancer or one or more tumor characteristics. In some embodiments, detection of HSF1 protein expression or activation (e.g., using IHC) is then used to apply such correlation to additional tumors, e.g., for purposes of providing prognostic, predictive, diagnostic, or treatment selection information.

As noted above, HSF1 binds to heat shock elements (HSEs). In some embodiments an HSE comprises two or more adjacent inverted repeats of the sequence 5′-n₁GAAn₅-3′, where n₁ and n₅ are independently A, G, C, or T, so that a single inverted repeat consists of 5′-n₋₅TTCn₋₁n₁GAAn₅-3′(SEQ ID NO.1), wherein n₋₁ is complementary to n₁ and n₋₅ is complementary to n₅. In some aspects, the disclosure relates to the discovery that regulatory regions of HSF1-CP genes that are strongly bound in cancer cells but not in heat shocked cells are enriched for HSEs that comprise exactly 3 inverted repeats, e.g., each having the sequence 5′-n-₅TTCn₋₁n₁GAAn₅-3′(SEQ ID NO.1), wherein n₋₁ is complementary to n₁ and n₋₅ is complementary to n₅. In some embodiments at least one of the inverted repeats has the sequence 5′-AGAAn₅-3′, so that a single inverted repeat consists of ‘5’-n₋₅TTCTAGAAn₅-3′(SEQ ID NO.2). In some embodiments at least one of the inverted repeats has the sequence 5′-GGAA n₅-3′, so that a single inverted repeat consists of 5′-n₋₅TTCCGGAAn₅-3′(SEQ ID NO.3). In some embodiments 2 of the inverted repeats are directly adjacent to each other (i.e., there are no intervening nucleotides). In some embodiments each of the inverted repeats is directly adjacent to at least one other inverted repeat. In some aspects, the disclosure relates to the discovery that regulatory regions of HSF1-CP genes that are strongly bound in cancer cells but not in heat shocked cells are enriched for binding sites for the transcription factor YY1 (Gene ID: 7528 (human); Gene ID: 22632 (mouse)). YY1 is a widely or ubiquitously distributed transcription factor belonging to the GLI-Kruppel class of zinc finger proteins and is involved in repressing and activating a diverse number of promoters. YY1 may direct histone deacetylases and histone acetyltransferases to a promoter in order to activate or repress the promoter, thus histone modification may play a role in the function of YY1. In some embodiments a YY binding site comprises or consists of GCnGCCA, wherein n is A, G, C, or T. In some aspects, the disclosure relates to the discovery that regulatory regions strongly bound in heat-shocked cells but not cancer cells are enriched for expanded HSEs, containing a fourth inverted repeat of 5′-n₁GAAn₅-3′ and for binding sites for the transcription factor AP1/Fos (NFE2L2). In some embodiments an AP1/Fos (NFE2L2) binding element comprises or consists of TGACTnA, wherein n is A, G, C, or T. In some embodiments n is C or A. In some aspects, the disclosure provides methods based, in some embodiments, at least in part on the identification of distinct patterns of transcription factor binding sites in genes that are strongly bound by HSF1 in cancer cells versus in heat-shocked cells. In some embodiments, methods of monitoring HSF1 cancer-related activity and methods of identifying modulators of HSF1 cancer-related activity are provided. In some embodiments reporter constructs are provided. In some embodiments, such methods and reporter constructs allow monitoring of HSF1 activity and/or identification of HSF1 modulators that are at least somewhat specific for HSF1 activity in cancer cells relative to heat shocked cells. For example, such modulators may inhibit HSF1 activity in cancer cells to a significantly greater extent than in heat shocked control cells and/or may selectively inhibit HSF1 binding or regulation of genes that are more strongly bound in cancer cells than in heat shocked control cells as compared with genes that are less strongly bound in cancer cells than in heat shocked control cells.

In some aspects, the invention provides an isolated nucleic acid comprising at least one YY binding site and an HSE that comprises exactly 3 inverted repeats. In some embodiments the sequence of the isolated nucleic acid comprises the sequence of at least a portion of a regulatory region of a Group A gene, Group B gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed control cells. In some embodiments, the sequence of the isolated nucleic acid comprises the sequence of at least a portion of a promoter region of a Group A gene, Group B gene, Module 1 gene, Module 2 gene, Module 3 gene, Module 4 gene, Module 5 gene, refined HSF1-CSS gene, or HSF1-CSS gene that is more highly bound by HSF1 in cancer cells than in heat shocked non-transformed control cells. In some embodiments the gene is positively regulated by HSF1 in cancer cells. In some embodiments the gene is strongly bound in cancer cells and weakly bound or not bound in non-transformed heat shocked control cells. In some embodiments, the sequence of the isolated nucleic acid comprises the sequence of at least a portion of a distal regulatory region of a Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed control cells. In some embodiments the gene is negatively regulated by HSF1 in cancer cells.

In some embodiments the invention provides an isolated nucleic acid comprising at least a portion of a regulatory region of a Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed cells, wherein the at least a portion of a regulatory region comprises an HSE. In some embodiments the isolated nucleic acid comprises at least a portion of a regulatory region of a Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed cells, wherein the at least a portion of a regulatory region comprises an HSE. In some embodiments the sequence of the nucleic acid comprises the sequence of at least a portion of a promoter region of a Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed control cells. In some embodiments the gene is positively regulated by HSF1 in cancer cells. In some embodiments the gene is strongly bound in cancer cells and weakly bound or not bound in non-transformed heat shocked control cells. In some embodiments the gene is HSPA8. In some embodiments the gene is CKS2, LY6K, or RBM23. In some embodiments an HSF1-CP gene is among the 5%, 10%, 20%, 30%, 40%, or 50% genes that are most highly bound by HSF1 in cancer cells, e.g., in metastatic cancer cells such as BPLER cells. In some embodiments, the sequence of the isolated nucleic acid comprises the sequence of at least a portion of a distal regulatory region of a Group A gene, Module 1 gene, Module 2 gene, Module 3 gene, Module 4 gene, Module 5 gene, HSF1-CaSig2 gene, HSF1-CaSig3 gene, refined HSF1-CSS gene, or HSF1-CSS gene that is more highly bound by HSF1 in cancer cells than in heat shocked non-transformed control cells. In some embodiments the gene is negatively regulated by HSF1 in cancer cells. In some embodiments the HSE comprises exactly 3 inverted repeats and, in some embodiments, further comprises a YY1 binding site. The HSE and YY binding site can be positioned in any order in various embodiments. In some embodiments the HSE and YY binding site are separated by up to 50 nt, 100 nt, 200 nt, 500 nt, 1 kB, 2 kB, 3 kB, 4 kB, 5 kB, 6 kB, 7 kB, 8 kB, 9 kB, or 10 kB.

In some embodiments of any of the afore-mentioned isolated nucleic acids, the isolated nucleic acid does not comprise an AP1/Fos (NFE2L2) binding site.

In some embodiments any of the afore-mentioned isolated nucleic acids comprise a binding site for RNA polymerase II and sufficient nucleic acid sequences for assembly of a transcription pre-initiation complex (Lee T I, Young R A (2000). “Transcription of eukaryotic protein-coding genes”. Annu. Rev. Genet. 34: 77-137; Kornberg R D (2007). “The molecular basis of eukaryotic transcription”. Proc. Natl. Acad. Sci. U.S.A. 104 (32): 12955-61).

In some embodiments an isolated nucleic acid is between 50 nucleotides (nt) and 20 kB long. In some embodiments an isolated nucleic acid is at least 100 nt, 200 nt, 500 nt, 1 kB, 2 kB, 3 kB, or 5 kB long and/or the isolated nucleic acid is up to 500 nt, 1 kB, 2 kB, 3 kB, 4 kB, 5 kB, 10 kB, or 20 kB long. All specific lengths and ranges are expressly contemplated. For example, in some embodiments the isolated nucleic acid is between 200 nt and 500 nt, between 500 nt and 1 kB, between 1 kB and 2 kB, between 2 kB and 3 kB, between 3 kB and 4 kB between 4 kB and 5 kB, between 5 kB and 10 kB etc. In some embodiments an isolated nucleic acid comprises at least a portion of a transcribed region of an HSF1-CP gene. In some embodiments an isolated nucleic acid comprises at least a portion of a coding region of an HSF1-CP gene. In some embodiments an isolated nucleic acid does not comprises a portion of a transcribed region of an HSF1-CP gene. For example, in some embodiments the sequence of an isolated nucleic acid comprises a sequence that lies upstream of (5′ with respect to) the transcription start site of an HSF1-CP gene. In some embodiments an isolated nucleic acid does not comprise a portion of a coding region of an HSF1-CP gene. In some embodiments the sequence of an isolated nucleic acid comprises a sequence that lies downstream of (3′ with respect to) the coding region, polyadenylation site, or transcribed portion of an HSF1-CP gene.

In some embodiments an isolated nucleic acid comprises at least a portion of a regulatory region of an HSF1-CP gene. In some aspects, a regulatory region comprises any nucleic acid sequence on the same piece of DNA as a transcription start site (TSS) of a gene that affects, e.g., direct, enhances, or represses transcription originating from such TSS. In some embodiments a regulatory region is located within 20 kB upstream or downstream of a TSS. In some embodiments a regulatory region is located within 20 kB upstream or downstream of a transcription termination site or DNA sequence corresponding to a polyadenylation site of a transcribed RNA. In some embodiments a regulatory region is located within 10 kB upstream or downstream of a TSS. In some embodiments a regulatory region is located within 10 kB upstream or downstream of a transcription termination site or DNA sequence corresponding to a polyadenylation site of a transcribed RNA. In some embodiments a regulatory region comprises a promoter region, comprising, e.g., a binding site for an RNA polymerase II and sufficient nucleic acid sequences for assembly of a transcription pre-initiation complex. In some embodiments a promoter region is located within −8 kB to +2 kB of the transcription start site (TSS) of a gene. In some embodiments a promoter region is located within −7 kB, −6 kB, −5 kB, −4 kB, −3 kB, or −2 kB, up to the TSS, +1 kB, or +2 kB of the TSS of a gene. In some embodiments a regulatory region is a distal regulatory region. In some embodiments a distal regulatory region is located beyond 2 kB and up to 8 kB downstream of the end of the coding region, end of the transcribed portion of a gene, or DNA sequence corresponding to a polyadenylation site of an RNA transcribed from such gene. In some embodiments the sequence of an isolated nucleic acid comprises or consists of a sequence that lies within −8, −6, −5, or −2 kb from the transcription start site (TSS) to either +5, +6, +8, or +10 kb from the TSS of an HSF1-CP gene. In some embodiments the sequence of an isolated nucleic acid comprises or consists of a sequence that lies within −8, −6, −5, or −2 kb from the transcription start site (TSS) to either +2, +5, +6, or +8 10 kb from the end of a coding region, end of the transcribed portion of an HSF1-CP gene, or DNA sequence corresponding to a polyadenylation site of an RNA transcribed from such gene. The sequence may be of any of the lengths mentioned in the preceding paragraph, in various embodiments.

In some aspects, the invention provides a nucleic acid construct comprising any of the afore-mentioned isolated nucleic acids and a nucleic acid sequence that encodes a reporter molecule. Such a nucleic acid construct may be referred to herein as an HSF1-CP reporter. A reporter molecule may comprise any genetically encodable detectable label (RNA or protein). In some embodiments, the reporter molecule is operably linked to the nucleic acid comprising an HSE. In some aspects, the invention provides vectors comprising any of the afore-mentioned isolated nucleic acids or nucleic acid constructs.

In some aspects, the invention provides cells comprising any of the afore-mentioned isolated nucleic acids, nucleic acid constructs, or vectors. A cell may be prokaryotic (e.g., bacterial) or eukaryotic (e.g., fungal, insect, vertebrate, avian, mammalian, human, etc.). In some embodiments a cell is of a species that is known to get cancer, e.g., an avian or mammalian cell. In some embodiments a prokaryotic, fungal, plant, or insect cell may be useful to, e.g., propagate a vector, produce a molecule, identify a protein-protein interaction, etc. In some embodiments a cell is a primary cell, non-immortal cell, immortal cell, non-cancer cell, or cancer cell. In some embodiments the nucleic acid construct or vector (or at least a portion thereof comprising the HSEs and the sequence encoding the reporter molecule) is integrated into the genome of the cell. In some embodiments cell lines derived from the cell or from a population of such cells are provided. In some embodiments any cell or cell line may be genetically modified by introducing a nucleic acid or vector encoding a polypeptide comprising HSF1 or a variant or fragment thereof. In some embodiments the nucleic acid encoding HSF1 is operably linked to expression control elements (e.g., a promoter) sufficient to direct expression in the cell. In some embodiments expression is regulatable, e.g., inducible. In some embodiments the polypeptide is a fusion protein comprising HSF1 or a variant or fragment thereof and a heterologous polypeptide. In some embodiments the heterologous polypeptide comprises a detectable protein or epitope tag. The heterologous polypeptide may be used, e.g., to assess HSF1 expression or localization, monitor alterations in HSF1 expression or localization over time, to isolate HSF1 from cells, etc. In some embodiments, the cell's endogenous HSF1 gene may be mutated or at least in part deleted. In some embodiments an HSF1 variant is a functional variant. In some embodiments an HSF1 variant is at least 90%, 95%, 96%, 97%, 98%, 99%, 99.5%, or more identical to HSF1 across at least 50%/., 60%, 70%, 80%, 90%, 95%, 96%, 97%, 98%, 99%, 99.5%, or the full length of HSF1. In some embodiments computer programs such as BLAST2, BLASTN, BLASTP, Gapped BLAST, etc., may be used to generate alignments and/or to obtain a percent identity (See, e.g., Karlin and Altschul, Proc. Natl. Acad. Sci. USA 87:22264-2268, 1990; Karlin and Altschul, Proc. Natl. Acad Sci. USA 90:5873-5877,1993; Altschul, et al., J. Mol. Biol. 215:403-410, 1990; Altschul, et al. Nucleic Acids Res. 25: 3389-3402, 1997). When utilizing such programs, the default parameters of the respective programs may be used. See the Web site having URL www.ncbi.nlm.nih.gov and/or McGinnis, S. and Madden, T L, W20-W25 Nucleic Acids Research, 2004, Vol. 32, Web server issue. In some embodiments no more than 20%, 10%, 5%, or 1% of positions in either sequence or in both sequences over a window of evaluation are occupied by a gap.

In some aspects, a cell comprising an HSF1-CP reporter is useful to assess HSF1 cancer-related activity, to identify modulators of HSF1 cancer-related activity, or to assess or monitor the effect of any agent on HSF1 cancer-related activity. In some embodiments a cell contains at least two such isolated nucleic acids, nucleic acid constructs, or vectors, wherein the at least two isolated nucleic acids, nucleic acid constructs, or vectors each comprises at least a portion of a regulatory region of an HSF1-CP gene, and wherein the reporter molecules are distinguishable. In some embodiments, this allows, e.g., assessment of expression regulated by each of multiple different regulatory regions of HSF1-CP genes in a given cell. In some embodiments a test agent that affects expression regulated by each of such regulatory regions is identified. In some embodiments a cell is a member of a population of cells, e.g., a population of cells obtained from a sample, or members of a cell line. It will be understood that various compositions disclosed herein may comprise a population of cells, and various methods herein may be practiced using a population of cells. For example, a measurement of DNA binding or a measurement of expression or assessing a test agent may be performed on or using a population of cells. Wherever relevant, aspects and embodiments pertaining to individual cells and aspects and embodiments pertaining to populations of cells are encompassed within the scope of the present disclosure. In some embodiments a population of cells is about 10, 10², 10³, 10⁴, 10⁵, 10⁶, 10⁷, 10⁸, 10⁹, cells, or more.

Certain aspects of the invention comprise or use a detectable label that comprises a detectable protein. For example, in some embodiments a reporter molecule comprises a detectable protein. In some embodiments a detectable protein comprises a fluorescent or luminescent protein. In some embodiments a detectable protein comprises an enzyme, e.g., an enzyme capable of catalyzing a reaction that converts a substrate to a detectable substance or otherwise produces a detectable event. Those of ordinary skill in the art will be aware of many such proteins and methods of detecting them and using them to, e.g., produce nucleic acid constructs useful for monitoring expression and/or monitoring activity of regulatory sequences contained in such constructs. Fluorescent proteins include, e.g., green fluorescent protein (GFP) from the jellyfish Aequorea victoria, related naturally occurring green fluorescent proteins, and related proteins such as red, yellow, and cyan fluorescent protein. Many of these proteins are found in diverse marine animals such as Hydrozoa and Anthozoa species, crustaceans, comb jellies, and lancelets. See, e.g., Chalfie, M. and Kain, S R (eds.) Green fluorescent protein: properties, applications, and protocols (Methods of biochemical analysis, v. 47). Wiley-Interscience, Hoboken, N.J., 2006, and/or Chudakov, D M, et al., Physiol Rev. 90(3):1103-63, 2010, for further information and references. In some embodiments, a detectable protein is monomeric. Examples of fluorescent proteins include Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima, mTomato. See Chudakov D M (cited above). In some embodiments a detectable protein comprises a luciferase. “Luciferase” refers to members of a class of enzymes that catalyze reactions that result in production of light. Luciferases are found in a variety of organisms including a variety of marine copepods, beetles, and others. Examples of luciferases include, e.g., luciferase from species of the genus Renilla (e.g., Renilla reniformis (Rluc), or Renilla mulleri luciferase), luciferase from species of the genus Gaussia (e.g., Gaussia princeps luciferase, Metridia luciferase from species of the marine copepod Metridia, e.g., Metridia longa, luciferase from species of the genus Pleuromamma, beetle luciferases (e.g. luciferase of the firefly Photinus pyralis or of the Brazilian click beetle Pyrearinus termitilluminans), etc. In some embodiments, a fluorescent or luminescent protein or luciferase is an engineered variant of a naturally occurring protein. Such variants may, for example, have increased stability (e.g., increased photostability, increased pH stability), increased fluorescence or light output, reduced tendency to dimerize, oligomerize, or aggregate, an altered absorption/emission spectrum (in the case of a fluorescent protein) and/or an altered substrate utilization. See, e.g., Chalfie, M. and Kain, S R (cited above) for examples. For example, the A. Victoria GFP variant known as enhanced GFP (eGFP) may be used. See, e.g., Loening, A M, et al., Protein Engineering, Design and Selection (2006) 19 (9): 391-400, for examples. In some embodiments a sequence is codon optimized for expression in cells of interest, e.g., mammalian cells. In some embodiments a detectable protein comprises a signal sequence that directs secretion of the protein. In some embodiments the secreted protein is soluble. In some embodiments the secreted protein remains attached to the cell. In some embodiments a detectable protein lacks a functional signal sequence. In some embodiments a signal sequence is at least in part removed or modified to render it nonfunctional or is at least in part replaced by a signal sequence endogenous to or functional in cells of interest, e.g., mammalian cells.

In some aspects, the disclosure provides methods of identifying agents, genes, gene products, and/or pathways that modulate HSF1 activity in cancer cells. In some embodiments a regulator of HSF1 activity regulates HSF1 expression, activation, or otherwise alters at least one activity performed by HSF1 in cancer cells. An activity performed by HSF1 in cancer cells may be referred to herein as an “HSF1 cancer-related activity”. In some embodiments an HSF1 cancer-related activity comprises modulating (e.g., activating or repressing) transcription of an HSF1-CP gene. In some embodiments an HSF1 cancer-related activity comprises binding to a regulatory region of an HSF1-CP gene. In some embodiments an HSF1 cancer-related activity is specific to cancer cells. In some embodiments an HSF1 cancer-related activity is not specific to cancer cells. For example, the activity may occur both in cancer cells and in non-transformed cells subjected to stress, e.g., thermal stress. “Thermal stress” is used interchangeably herein with “heat shock” and refers to exposing cells to elevated temperature (i.e., temperature above physiologically normal) for a sufficient period of time to detectably, e.g., robustly, induce the heat shock response. In some embodiments heat shock comprises exposing cells to a temperature of 42±0.5 degrees C. for about 1 hour or similar exposures to elevated temperatures (above 40 or 41 degrees C.) resulting in similar or at least approximately equivalent induction of the heat shock response. In some embodiments cells are allowed to recover for up to about 60 minutes, e.g., about 30 minutes, at sub-heat shock temperature, e.g., 37 degrees C., prior to isolation of RNA or DNA. In some embodiments assessment of the effect of heat shock on expression may occur after allowing an appropriate amount of time for translation of a transcript whose expression is induced by HSF1.

In some embodiments the level of an HSF1 activity is expressed as an absolute level. In some embodiments the level of an HSF1 activity is expressed as a relative level. For example, activation or repression of an HSF1-CP gene by HSF1 in cancer cells may be expressed as a fold-increase or fold-decrease in expression relative to a reference value. In some embodiments a reference value for a level of an activity is the level of the relevant activity in non-cancer cells not subjected to heat shock. In some embodiments a reference value is the level of the relevant activity in cells in which expression or activity of functional HSF1 is inhibited.

In some embodiments an HSF1 cancer-related activity is detectable in cancer cells and is not detectable in heat shocked non-cancer cells. In some embodiments the level of an HSF1 cancer-related activity is detectably greater in cancer cells than in heat shocked non-cancer cells and is not detectably greater in heat-shocked non-cancer cells than in non-cancer cells maintained under normal conditions. In some embodiments an HSF1 cancer-related activity is detectable in cancer cells and in heat shocked non-cancer cells. In some embodiments the level of an HSF1 cancer-related activity is significantly greater in cancer cells and in heat shocked non-cancer cells than in non-cancer cells maintained under normal conditions. In some embodiments the level of an HSF1 cancer-related activity is greater in cancer cells than in non-cancer cells subjected to heat shock. In some embodiments a first level (e.g., a level of an HSF1 cancer-related activity in cancer cells) is greater than a second level (e.g., a level of an HSF1 cancer-related activity in non-cancer cells) by a statistically significantly amount. In some embodiments a first level is greater than a second level by a factor of at least 1.1., 1.2, 1.3, 1.4, 1.5, 1.75, 2.0, 2.5, 3.0, 4.0, 5.0, 7.5, 10, 15, 20, 25, 50, 100, or more.

Modulators of HSF1 Cancer-Related Activity

In addition to its value in classification and prognosis, HSF1 is a promising target for cancer therapeutics. The protein's widespread activation in many different tumor types augurs a broad range of clinical applications. In this regard, the homogeneity of HSF1 expression throughout entire sections of tumors is notable. Pre-existing heterogeneities for the expression of many recently identified therapeutic targets has emerged as a major factor contributing to the emergence of resistance (Gerlinger et al., 2012). Without wishing to be bound by any theory, the uniform reliance of cancer cells on HSF1 activity for proliferation and survival suggests that HSF1-targeted therapeutics may be less susceptible to this liability.

In some aspects, the invention provides methods of identifying candidate modulators (e.g., candidate inhibitors or enhancers) of HSF1 cancer-related activity. In some embodiments a method of identifying a candidate modulator of HSF1 cancer-related activity comprises: (a) providing a nucleic acid comprising at least a portion of a regulatory region a gene, wherein the regulatory region is bound by HSF1 in cancer cells; (b) contacting the nucleic acid with a test agent; and (c) assessing the level of expression of the gene or the level of activity of a gene product of the gene, wherein the test agent is identified as a candidate modulator of HSF1 activity if the level of expression of the gene or the level of activity of a gene product of the gene differs from a control level. In some embodiments the method comprises providing a cell that contains the nucleic acid construct and contacting the cell with the test agent. In some embodiments the cell is a tumor cell. In some embodiments the regulatory region is operably linked to a nucleic acid sequence that encodes a reporter molecule, and assessing the level of expression of the gene comprises assessing the level or activity of the reporter molecule.

In some embodiments a method of identifying a candidate modulator of HSF1 cancer-related activity comprises steps of: (a) contacting a cell that expresses HSF1 with a test agent; (b) measuring the level of an HSF1 cancer-related activity exhibited by the cell; and (c) determining whether the test agent modulates the HSF1 cancer-related activity, wherein a difference in the level of the HSF1 cancer-related activity in the presence of the test agent as compared to the level in the absence of the test agent identifies the agent as a candidate modulator of HSF1 cancer-related activity. In some embodiments the HSF1 cancer-related activity is binding to a regulatory region of a HSF1-CP gene. In some embodiments the HSF1 cancer-related activity is expression of a HSF1-CP gene. In some embodiments the HSF1-CP gene is a Group A gene, Group B gene, HSF1-CSS gene, HSF1-CaSig2 gene, HSF1-CaSig3 gene, refined HSF1-CSS gene, Module 1 gene, Module 2 gene, Module 3 gene, Module 4 gene, or Module 5 gene, wherein the gene is more highly bound by HSF1 in cancer cells than in heat shocked non-transformed control cells. In some embodiments the HSF1 cancer-related activity is measured by measuring expression of an HSF1-CP reporter. In some embodiments an HSF1 cancer-related activity exhibited by a cell may be assessed while the cell is alive (e.g., by detecting a fluorescent reporter molecule). In some embodiments an HSF1 cancer-related activity exhibited by a cell may be assessed in a sample obtained from the cell (e.g., DNA, RNA, cell lysate, etc.).

In some embodiments, a test agent is identified as an inhibitor of HSF1 cancer-related activity if it inhibits binding of HSF1 to a regulatory region of at least 1, 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, Group B genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes or inhibits expression of one or more genes that are positively regulated by HSF1 in cancer cells or increases expression of one or more genes that are negatively regulated by HSF1 in cancer cells.

In some embodiments any of the methods comprises comparing the effect of a test agent on HSF1 binding to, or regulation of, an HSF1-CP gene in cancer cells and in heat shocked non-transformed control cells. In some embodiments the HSF1-CP gene is one that is bound in both cancer cells and in heat shocked non-transformed control cells. Such methods may be used, e.g., to identify agents that selectively affect, e.g., inhibit, HSF1 activity in cancer cells.

The term “agent” is used interchangeably with “compound” herein. Any of a wide variety of agents may be used as a test agent in various embodiments. For example, an agent, e.g., a test agent, may be a small molecule, polypeptide, peptide, nucleic acid, oligonucleotide, lipid, carbohydrate, or hybrid molecule. In some embodiments an oligonucleotide comprises an siRNA, shRNA, antisense oligonucleotide, aptamer, or random oligonucleotide. In some embodiments a cDNA comprises a full length cDNA. In some embodiments a cDNA comprises a portion of a full length cDNA, wherein the portion retains at least some of the functional activity of the full length cDNA.

Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A compound library may comprise natural products and/or compounds generated using non-directed or directed synthetic organic chemistry. In some embodiments a library is a small molecule library, peptide library, peptoid library, cDNA library, oligonucleotide library, or display library (e.g., a phage display library). In some embodiments a library comprises agents of two or more of the foregoing types. In some embodiments oligonucleotides in an oligonucleotide library comprise siRNAs, shRNAs, antisense oligonucleotides, aptamers, or random oligonucleotides.

A library may comprise, e.g., between 100 and 500,000 compounds, or more. In some embodiments a library comprises at least 10,000, at least 50,000, at least 100,000, or at least 250,000 compounds. In some embodiments compounds of a compound library are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments compounds that have been identified as “hits” or “leads” in a drug discovery program and/or analogs thereof. In some embodiments a library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments, an “approved human drug” or compound collection comprising one or more approved human drugs is tested. An “approved human drug” is an agent that has been approved for use in treating humans by a government regulatory agency such as the US Food and Drug Administration, European Medicines Evaluation Agency, or a similar agency responsible for evaluating at least the safety of therapeutic agents prior to allowing them to be marketed. A test agent may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments an agent has undergone at least some preclinical or clinical development or has been determined or predicted to have “drug-like” properties. For example, an agent may have completed a Phase I trial or at least a preclinical study in non-human animals and shown evidence of safety and tolerability. In some embodiments an agent is not an agent that is found in a cell culture medium known or used in the art, e.g., for culturing vertebrate, e.g., mammalian cells, e.g., an agent provided for purposes of culturing the cells, or, if the agent is found in a cell culture medium known or used in the art, the agent may be used at a different, e.g., higher, concentration when used in a method or composition described herein. In some embodiments a test agent is not an agent known in the art as being useful for treating tumors (e.g., for inhibiting tumor cell survival or proliferation or for inhibiting tumor maintenance, growth, or progression) or for treating side effects associated with chemotherapy. In some embodiments a test agent is not a compound that binds to and inhibits Hsp90. In some embodiments a test agent has at least one known target or biological activity or effect. For example, the test agent may be a receptor ligand (e.g., an agonist or antagonist), enzyme inhibitor (e.g., a kinase inhibitor). In some embodiments a test agent is capable of binding to HSF1 or is tested for ability to bind to HSF1. In some embodiments the HSF1 is purified from cancer cells.

In some embodiments the effect of overexpression or knockdown (reduced expression) of one or more genes on an HSF1 cancer-related activity is assessed. In some embodiments one or more cDNAs, RNAi agents (e.g., siRNAs, microRNAs, or shRNAs), or antisense agents whose sequence corresponds to a gene is used as a test agent. In some embodiments the cDNA, RNAi agent, or antisense agent is directly introduced into cells. In some embodiments the cDNA, RNAi agent, or antisense agent is introduced into cells by introducing a nucleic acid construct or vector comprising a sequence that encodes the cDNA, RNAi agent, or antisense agent, operably linked to appropriate expression control elements (e.g., a promoter) to direct expression in cells of interest. The cDNA, RNAi agent, or antisense agent is then expressed intracellularly. In some embodiments, if cells into which the cDNA, RNAi agent, or antisense agent is introduced exhibit an alteration in expression of an HSF1 reporter molecule or exhibit altered HSF1 activity, the agent is identified as a candidate modulator of HSF1 cancer-related activity. In some embodiments, if cells into which the cDNA, RNAi agent, or antisense agent is introduced exhibit an alteration exhibit an alteration in expression of an HSF1 reporter molecule or exhibit altered HSF1 activity, the gene to which the agent corresponds is identified as a candidate genetic modifier of HSF1 cancer-related activity. In some embodiments, if cells into which the cDNA, RNAi agent, or antisense agent is introduced exhibit an alteration in expression of an HSF1 reporter molecule or exhibit altered HSF1 activity, a gene product of the gene to which the agent corresponds is identified as a candidate modulator of HSF1 cancer-related activity. In some embodiments a library of such agents is tested. In some embodiments the library comprises test agents whose sequences correspond to at least 50%, 60%, 70%, 80%, 90%, 95%, 96%, 97%, 98%, 99%, or more (e.g., all) of the genes in the genome of an organism or species of interest (e.g., human, mouse). In some embodiments the library comprises test agents whose sequences correspond to at least 50%, 60%, 70%, 80%, 90%, 95%, 96%, 97%, 98%, 99%, or more (e.g., all) of the members of a focused subset of the genes in the genome of an organism or species of interest (e.g., human, mouse), wherein the focused subset consists of genes that can be classified into the same functional category, have the same or a similar biochemical activity (e.g., catalyze the same biochemical reaction), participate in the same pathway or process etc. Examples of focused subsets include kinases (e.g., protein kinases), phosphatases, chromatin modifying enzymes, transcription factors, transcriptional co-regulators, G protein coupled receptors, small GTPases, cell surface receptors, signal transduction proteins, and subsets of any of the foregoing. It will be understood that a gene may fall into multiple subsets.

In some embodiments, a method is of use to identify one or more genes and/or gene products that regulate HSF1. In some embodiments gene products that play a direct or indirect role in expression, post-translational modification, or nuclear localization, of HSF1 (and/or genes that encode such gene products) may be identified. For example, a kinase that phosphorylates HSF1 and thereby regulates (e.g., activates) HSF1 activity may be identified. In some embodiments gene products that physically interact with HSF1 (and/or genes that encode such gene products) may be identified. For example, a transcriptional co-activator that cooperates with HSF1 to activate or repress transcription of one or more HSF1-CP genes may be identified. In some embodiments, such proteins are targets for drug development.

In some aspects, disclosed herein are methods of identifying a post-translational modification of HSF1, wherein the post-translational modification potentially regulates HSF1 cancer-related activity. As used herein, the term “post-translational modification” (PTM) encompasses any alteration to a polypeptide that occurs in cells during or after translation of mRNA that encodes the polypeptide. Examples of PTMs include covalent addition of a moiety to a side chain or terminus (e.g., phosphorylation, glycosylation, SUMOylation, methylation, acetylation, acylation (e.g., fatty acid acylation), ubiquitination, Neddylation), altering the chemical identity of an amino acid, or site-specific cleavage. In some embodiments a PTM is catalyzed by a cellular enzyme. A PTM may be described by the name of the particular modification and the site (position) within the polypeptide at which the modification occurs. A “PTM pattern” refers to the presence of a PTM at each of two or more sites in a single protein molecule. PTMs in a PTM pattern may be the same (e.g., phosphorylation at each of multiple sites) or at least some of them may differ (e.g., a phosphorylation at a first site and a SUMOylation at a second site). A site of potential post-translational modification is any site that is compatible with being post-translationally modified. For example, serine, threonine, tyrosine, and histidine residues are potential phosphorylation sites in eukaryotic cells. In some embodiments a PTM site occurs within a consensus sequence for an enzyme that catalyzes the PTM.

In some embodiments a method of identifying a PTM of HSF1 comprises identifying PTMs or PTM patterns that differ in HSF1 in or isolated from cancer cells as compared to HSF1 in or isolated from non-cancer cells comprises: (a) comparing the extent to which a PTM or PTM pattern occurs in HSF1 of cancer cells with the extent to which it occurs in HSF1 of non-cancer cells, and (b) identifying the PTM or PTM pattern as a PTM or PTM pattern that differs in cancer if the extent to which the PTM or PTM pattern occurs in HSF1 of cancer cells differs from the extent to which it occurs in HSF1 of non-cancer cells. In some embodiments, step (b) comprises (i) obtaining HSF1 isolated from cancer cells and measuring the PTM or PTM pattern; and (ii) obtaining HSF1 isolated from non-cancer cells and measuring the s the PTM or PTM pattern. In some embodiments a historical value is used for either or both measurements of the PTM or PTM pattern. In some embodiments the method comprises isolating HSF1 from cancer cells and/or non-cancer cells. In some embodiments cancer cells and/or non-cancer cells are subjected to heat shock for at least a period of time within the 1, 2, 3, 4, 6, 8, 12, 16, 24, 36, or 48 hours prior to isolation of HSF1. In some embodiments cancer cells and non-cancer cells are not subjected to heat shock within the 1, 2, 3, 4, 6, 8, 12, 16, 24, 36, or 48 hours prior to isolation of HSF1 or, if subjected to heat shock within such time period, have returned to a state that does not differ significantly from that of non-heat shocked cells. Any suitable method can be used to identify or measure a PTM or PTM pattern. Useful methods include, e.g., amino acid sequencing, peptide mapping, use of modification state-specific antibodies or other binding agents, mass spectrometry (MS) analysis (e.g., MS/MS), etc. In some embodiments site-directed mutagenesis is used to identify a PTM that affects HSF1 cancer-related activity. For example, an amino acid that is a site of PTM in cancer cells may be altered to a different amino acid that is not post-translationally modified. The variant may be tested for at least one HSF1 cancer-related activity. If the alteration affects HSF1 cancer-related activity, then the PTM is of potential functional significance to HSF1 cancer-related activity. In some embodiments, a gene product that catalyzes a functionally significant HSF1 PTM is a target of interest for drug development. In some embodiments a PTM or PTM pattern comprises phosphorylation at S121, S230, S292, S303, S307, S314, S319, S326, S344, S363, S419, and/or S444.

In some aspects, disclosed herein are methods of identifying PTMs or PTM patterns that affect the localization or activity of HSF1 in cancer cells. In some embodiments a PTM or PTM pattern selectively affects localization or activity of HSF1 in cancer cells. The PTM or PTM pattern may occur differentially in cancer cells as compared to non-cancer cells and/or may have a different effect on HSF1 localization or activity in cancer cells as compared to its effect in non-cancer cells.

In some aspects, disclosed herein are methods of identifying intracellular molecules, e.g., RNAs or proteins, that interact with HSF1, e.g., in a cancer-specific manner. Any of a variety of methods for detecting protein-protein interactions or protein-RNA interactions may be used. In some embodiments such molecules may be identified by immunoprecipitating HSF1 in cancer cells and in non-transformed heat shocked cells, and identifying molecules that are enriched or specifically present in HSF1 immunoprecipitates from cancer cells as compared with HSF1 immunoprecipitates from non-transformed heat shocked cells. In some embodiments a method comprises performing a two-hybrid screen using HSF1 as a bait in cancer cells and in non-cancer heat shocked control cells, and identifying molecules that are enriched or specifically interact with HSF1 in cancer cells as compared with HSF1 in non-transformed heat shocked cells. In some embodiments a protein fragment complementation assay or a luminescence-based mammalian interactome mapping (LUMIER) assay may be used. In some embodiments a fusion protein comprising (a) HSF1 or a variant or fragment thereof; and (b) a detectable protein is used.

In some embodiments a high throughput screen (HTS) is performed. High throughput screens often involve testing large numbers of test agents with high efficiency, e.g., in parallel. For example, tens or hundreds of thousands of agents may be routinely screened in short periods of time, e.g., hours to days. Such screening is often performed in multiwell plates (sometimes referred to as microwell or microtiter plates or microplates) containing, e.g., 96, 384, 1536, 3456, or more wells or other vessels in which multiple physically separated depressions, wells, cavities, or areas (collectively “wells”) are present in or on a substrate. Different test agent(s) may be present in or added to the different wells. It will be understood that some wells may be empty, may comprise replicates, or may contain control agents or vehicle. High throughput screens may involve use of automation, e.g., for liquid handling, imaging, and/or data acquisition or processing, etc. In some embodiments an integrated robot system comprising one or more robots transports assay-microplates from station to station for, e.g., addition, mixing, and/or incubation of assay constituents (e.g., test agent, target, substrate) and, in some embodiments, readout or detection. A HTS system may prepare, incubate, and analyze many plates simultaneously. Certain general principles and techniques that may be applied in embodiments of a HTS are described in Macarrón R & Hertzberg R P. Design and implementation of high-throughput screening assays. Methods Mol Biol., 565:1-32, 2009 and/or An W F & Tolliday N J., Introduction: cell-based assays for high-throughput screening. Methods Mol Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg H\ddot{ν}ser. Test agent(s) showing an activity of interest (sometimes termed “hits”) may be retested and/or, optionally (e.g., depending at least in part on results of retesting) selected for further testing, development, or use.

In some embodiments one or more “confirmatory” or “secondary” assays or screens may be performed to confirm that a test agent identified as a candidate modulator in an initial (“primary”) assay or screen modulates a target molecule of interest (e.g., HSF1) or modulates an activity of interest (e.g., HSF1 cancer-related activity) or to measure the extent of modulation or to assess specificity. Confirmatory testing may utilize the same assay or a different assay as that used to identify the test agent. The exact nature of the confirmatory testing may vary depending on a variety of factors such as the nature of the primary assay, the nature of the candidate modulator, etc. One of ordinary skill in the art will be able select one or more assays sufficient to reasonably confirm to the satisfaction of those of ordinary skill in the art that an agent indeed modulates a selected target molecule or activity of interest. In some embodiments a candidate modulator that has given satisfactory results upon confirmatory testing may be referred to as a “confirmed modulator”. In some embodiments a test agent that exhibits a reasonable degree of specificity for a selected target molecule (e.g., HSF1) or activity of interest (e.g., HSF1 cancer-related activity) may be identified or selected, e.g., for further testing or development or use.

In some embodiments one or more agents identified as a candidate modulator or confirmed modulator of HSF1 cancer-related activity may be selected for, e.g., further testing, development, or use. For example, an agent that is determined or predicted to have higher potency, greater selectivity for a target of interest (e.g., HSF1 or an endogenous regulator of HSF1), one or more drug-like properties, potential for useful modification, or any other propert(ies) of interest, e.g., as compared with one or more other hits, e.g., as compared with the majority of other hits, may be selected. A selected agent may be referred to as a “lead”. Further testing may comprise, e.g., resynthesis or re-ordering of a hit, retesting of the original hit preparation or resynthesized or newly ordered preparation in the same or a different assay, etc. Development of an agent may comprise producing an altered agent. In some embodiments a pharmacophore is identified based on structures of multiple hit compounds, which may be used to design additional compounds (e.g., structural analogs). In some embodiments any of the methods may comprise producing an altered agent, e.g., an altered lead agent. In some embodiments a method comprises modifying an agent to achieve or seek to achieve an alteration in one or more properties, e.g., (1) increased affinity for a target of interest; (2) decreased affinity for a non-target molecule, (3) increased solubility (e.g., increased aqueous solubility); (4) increased stability (e.g., in vivo); (5) increased potency; (6) increased selectivity, e.g., for a target molecule or for tumor cells, e.g., a higher selectivity for tumor versus non-tumor cells; (7) a decrease in one or more side effects (e.g., decreased adverse side effects, e.g., decreased toxicity); (8) increased therapeutic index; (9) one or modified pharmacokinetic properties (e.g., absorption, distribution, metabolism and/or excretion); (10) modified onset of therapeutic action or duration of effect; (11) modified, e.g., increased, oral bioavailability; (12) modified, e.g., increased, tissue or tumor penetration; (13) modified, e.g., increased, cell permeability; (14) modified, e.g., increased, delivery to a selected subcellular organelle; (15) modified, e.g., increased, increased ability to cross the blood-brain barrier (increased ability to cross the blood-brain barrier may be desirable in some embodiments if use of the agent to treat central nervous system (CNS) tumors, e.g., brain tumors, is contemplated; decreased ability to cross the blood-brain barrier may be desirable in some embodiments if the agent has adverse effects on the CNS); (16) altered plasma protein binding (e.g., to albumin, alpha-1 acid glycoprotein, α, β, γ globulins, etc.).

In some embodiments any of the methods may further comprise determining an in vitro activity or in vivo activity or toxicology profile of an agent or altered agent. One or more additional alterations may be performed, e.g., based at least in part on such analysis. Multiple cycles of alteration and testing may be performed, thereby generating additional altered agents. In some embodiments any of the methods may further comprise performing a quantitative structure activity relationship analysis of multiple hit, lead, or altered agents. In some embodiments alteration may be accomplished through at least partly random or non-predetermined modification, predetermined modification, and/or using computational approaches. An altered agent, e.g., an altered lead agent, may be produced using any suitable method. In some embodiments an agent or an intermediate obtained in the course of synthesis of the agent may be used as a starting material for alteration. In some embodiments an altered agent may be synthesized using any suitable materials and/or synthesis route. In some embodiments alteration may make use of established principles or techniques of medicinal chemistry, e.g., to predictably alter one or more properties. In some embodiments, a first library of test agents is screened using any of the methods described herein, one or more test agents that are “hits” or “leads” is identified, and at least one such hit or lead is subjected to systematic structural alteration to create a second library of compounds structurally related to the hit or lead. In some embodiments the second library is then screened using methods described herein or other methods.

In some embodiments any of the methods may comprise producing an altered agent, e.g., an altered lead agent, by modifying an agent to incorporate or be attached to a label, which may optionally be used to detect or measure the agent or a metabolite of the agent, e.g., in a pharmacokinetic study. In some embodiments any of the methods may comprise producing an altered agent, e.g., an altered lead agent, by modifying an agent to incorporate or be attached to a second moiety (or more than two moieties). In some embodiments a second (or additional) moiety comprises a linker, tag, or targeting moiety. In some embodiments a second (or additional) moiety may modify one or more properties (1)-(16) listed above. In some embodiments a modification may cause increased delivery of the agent to or increased accumulation of the agent at a site of desired activity in the body of a subject. A site may be, e.g., a tumor, organ, tissue, or cell type.

In some embodiments any of the methods may comprise producing a composition by formulating an agent (e.g., a test agent, candidate HSF1 modulator, altered agent, candidate anti-tumor agent, etc.) or two or more agents with a pharmaceutically acceptable carrier.

In some embodiments any of the methods may comprise testing the effect of an agent (e.g., a test agent, candidate HSF1 modulator, altered agent, etc.) on one or more tumor cell lines. In some embodiments an agent is tested in a diverse set of cancers or cancer cell lines. Any cancer or cancer cell line can be used. Exemplary cancers and cancer cell lines are discussed herein. Tumor cells may be maintained in a culture system comprising a culture medium to which an agent is added or has been added. The effect of the agent on tumor cell viability, proliferation, tumor-initiating capacity, or any other tumor cell property may be assessed. In general, any suitable method known in the art may be used for assessing tumor cell viability or proliferation or tumor-initiating capacity in various embodiments. In certain embodiments survival and/or proliferation of a cell or cell population, e.g., in cell culture, may be determined by: a cell counting assay (e.g., using visual inspection, automated image analysis, flow cytometer, etc.), a replication assay, a cell membrane integrity assay, a cellular ATP-based assay, a mitochondrial reductase activity assay, a BrdU, EdU, or H3-Thymidine incorporation assay, a DNA content assay using a nucleic acid dye, such as Hoechst Dye, DAPI, Actinomycin D, 7-aminoactinomycin D or propidium iodide, a cellular metabolism assay such as resazurin (sometimes known as AlamarBlue or by various other names), MTT, XTT, and CellTitre Glo, etc., a protein content assay such as SRB (sulforhodamine B) assay; nuclear fragmentation assays; cytoplasmic histone associated DNA fragmentation assay; PARP cleavage assay; TUNEL staining; or annexin staining.

It will be understood that inhibition of cell proliferation or survival by a useful agent may or may not be complete. For example, cell proliferation may, or may not, be decreased to a state of complete arrest for an effect to be considered one of inhibition or reduction of cell proliferation. In some embodiments, “inhibition” may comprise inhibiting proliferation of a cell that is in a non-proliferating state (e.g., a cell that is in the GO state, also referred to as “quiescent”) and/or inhibiting proliferation of a proliferating cell (e.g., a cell that is not quiescent). Similarly, inhibition of cell survival may refer to killing of a cell, or cells, such as by causing or contributing to necrosis or apoptosis, and/or the process of rendering a cell susceptible to death. The inhibition may be at least about 10%, 15%, 20%, 25%, 30%, 35%, 40%, 45%, 50%, 55%, 60%, 65%, 70%, 75%, 80%, 85%, 90%, 95%, 99%, or 100% of a reference level (e.g., a control level). In some embodiments an agent is contacted with tumor cells in an amount (e.g., at a concentration) that inhibits tumor cell proliferation or survival by a selected amount, e.g., by at least about 10%, 15%, 20%, 25%, 30%, 35%, 40%, 45%, 50%, 55%, 60%, 65%, 70%, 75%, 80%, 85%, 90%, 95%, 99%, or 100% of a reference level (e.g., a control level).

In some embodiments an anti-tumor effect is inhibition of the capacity of tumor cells to form colonies in suspension culture. In some embodiments an anti-tumor effect is inhibition of capacity of the one or more tumor cells to form colonies in a semi-solid medium such as soft agar or methylcellulose. In some embodiments an anti-tumor effect is inhibition of capacity of the one or more tumor cells to form tumor spheres in culture. In some embodiments an anti-tumor effect is inhibition of the capacity of the one or more tumor cells to form tumors in vivo.

In some embodiments any of the methods may comprise testing an agent in vivo, by administering one or more doses of the agent to a subject, e.g., a subject harboring a tumor cell or tumor, and evaluating one or more pharmacokinetic parameters, evaluating the effect of the agent on the subject (e.g., monitoring for adverse effects) and/or evaluating the effect of the agent on the growth and/or survival of the cancer cell in the subject. It will be understood that the agent may be administered in a suitable composition comprising the agent. In some embodiments any of the methods may comprise testing an agent in a tumor model in vivo, by administering one or more doses of the composition to a non-human animal (“test animal”) that serves as a tumor model and evaluating the effect of the agent on the tumor in the subject. In some embodiments a test animal is a non-human mammal, e.g., a rodent such as a mouse, rat, hamster, rabbit, or guinea pig; a dog, a cat, a bovine or ovine, a non-human primate (e.g., a monkey such as a cynomolgus or rhesus monkey). By way of example, certain in vivo tumor models are described in U.S. Pat. No. 4,736,866; U.S. Ser. No. 10/990,993; PCT/US2004/028098 (WO/2005/020683); and/or PCT/US2008/085040 (WO/2009/070767). Introduction of one or more cells into a subject (e.g., by injection or implantation) may be referred to as “grafting”, and the introduced cell(s) may be referred to as a “graft”. In general, any tumor cells may be used in an in vivo tumor model in various embodiments. Tumor cells may be from a tumor cell line or tumor sample. In some embodiments tumor cells originate from a naturally arising tumor (i.e., a tumor that was not intentionally induced or generated for, e.g., experimental purposes). In some embodiments experimentally produced tumor cells may be used. The number of tumor cells introduced may range, e.g., from 1 to about 10, 10², 10³, 10⁴, 10⁵, 10⁶, 10⁷, 10⁸, 10⁹, or more. In some embodiments the tumor cells are of the same species or inbred strain as the test animal. In some embodiments the tumor cells may originate from the test animal itself. In some embodiments the tumor cells are of a different species than the test animal. For example, the tumor cells may be human cells. In some embodiments, a test animal is immunocompromised, e.g., in certain embodiments in which the tumor cells are from a different species to the test animal or originate from an immunologically incompatible strain of the same species as the test animal. For example, a test animal may be selected or genetically engineered to have a functionally deficient immune system or may be treated (e.g., with radiation or an immunosuppressive agent or surgery such as removal of the thymus) so as to reduce immune system function. In some embodiments, a test animal is a SCID mouse, NOD mouse, NOD/SCID mouse, nude mouse, and/or Rag1 and/or Rag2 knockout mouse, or a rat having similar immune system dysfunction. Tumor cells may be introduced at an orthotopic or non-orthotopic location. In some embodiments tumor cells are introduced subcutaneously, under the renal capsule, or into the bloodstream. Non-tumor cells (e.g., fibroblasts, bone marrow derived cells), an extracellular matrix component or hydrogel (e.g., collagen or Matrigel®), or an agent that promotes tumor development or growth may be administered to the test animal prior to, together with, or separately from the tumor cells. Tumor cells may be contacted with an agent prior to grafting and/or following grafting (by administering the agent to the test animal). The number, size, growth rate, metastasis, or other properties may be assessed at one or more time points following grafting. In some embodiments a tumor in an in vivo tumor model arises due to neoplastic transformation that occurs in vivo, e.g., at least in part as a result of one or more mutations existing or occurring in a cell in vivo. In some embodiments a test animal is a tumor-prone animal. The animal may, for example, be of a species or strain that naturally has a predisposition to develop tumors and/or may be a genetically engineered animal. For example, the animal may be a genetically engineered animal at least some of whose cells comprise, as a result of genetic modification, at least one activated oncogene and/or in which at least one tumor suppressor gene has been functionally inactivated. Standard methods of generating genetically modified animals, e.g., transgenic animals that comprises exogenous genes or animals that have an alteration to an endogenous gene, e.g., an insertion or an at least partial deletion or replacement (sometimes referred to as “knockout” or “knock-in” animal) may be used.

An agent may be administered by any route or regimen in various embodiments. For example, the agent can be administered prior to, concomitant with, and/or following the administration of tumor cells or development of a tumor. An agent can be administered regularly throughout the course of the testing period, for example, one, two, three, four, or more times a day, weekly, bi-weekly, or monthly, beginning before or after tumor cells have been administered, in other embodiments, the agent is administered continuously to the subject (e.g., intravenously or by release from an implant, pump, sustained release formulation, etc.). The dose of the agent to be administered can depend on multiple factors, including the type of agent, weight of the test animal, frequency of administration, etc. Determination of dosages is routine for one of ordinary skill in the art. In some embodiments doses are 0.01 mg/kg-200 mg/kg (e.g., 0.1-20 mg/kg or 1-10 mg/kg). The test animal may be used to assess effect of the agent or a combination of agents on tumor formation, tumor size, tumor number, tumor growth rate, progression (e.g., local invasion, regional or distant metastasis), etc. In some embodiments a non-human animal is used to assess efficacy, half-life, clearance, metabolism, and/or toxicity of an agent or combination of agents. Methods known in the art can be used for such assessment. For example, tumor number, size, growth rate, or metastasis may, for example, be assessed using various imaging modalities, e.g., X-ray, magnetic resonance imaging, functional imaging, e.g., of metabolism (e.g., using PET scan), etc. In some embodiments tumor(s) may be removed from the body (e.g., at necropsy) and assessed (e.g., tumors may be counted, weighed, and/or size (e.g., dimensions) measured). In some embodiments the size and/or number of tumors may be determined non-invasively. For example, in certain tumor models, tumor cells that are fluorescently labeled (e.g., by expressing a fluorescent protein such as GFP) can be monitored by various tumor-imaging techniques or instruments, e.g., non-invasive fluorescence methods such as two-photon microscopy. The size of a tumor implanted subcutaneously can be monitored and measured underneath the skin.

In some embodiments, an agent may be contacted with tumor cells ex vivo, and the tumor cells are then introduced into a test animal that serves as a tumor model. The ability of the agent to inhibit tumor development, tumor size, or tumor growth is assessed. The agent may or may not also be administered to the subject.

In some embodiments samples or data may be acquired at multiple time points, e.g., during or after a dose or series of doses. In some embodiments a suitable computer program may be used for data analysis, e.g., to calculate one or more pharmacokinetic parameters. In certain embodiments, the subject is a mouse, rat, rabbit, dog, cat, sheep, pig, non-human primate, or human.

In some aspects, a computer-readable medium is provided. In some embodiments a computer-readable medium stores at least some results of a screen to identify agents that modulate, e.g., inhibit, HSF1 cancer-related activity. The results may be stored in a database and may include one or more screening protocols, results obtained from a screen, predicted properties of hits, leads, or altered leads, or results of additional testing of hits, leads, or altered leads.

In some embodiments an agent capable of causing a decrease in level or activity of a target, e.g., HSF1, of at least 25%, 50%, 75%, 90%, 95%, 99%, or more when used in a suitable assay at a concentration equal to or less than approximately 1 mM, 500 μM, 100 μM, 50 μM, 10 μM, 5 μM, 1 μM, 500 nM, 100 nM, 50 nM, 10 nM, 5 nM, 1 nM, 0.5 nM, or 0.1 nM may be screened for, identified, produced, provided, or used.

In some embodiments an agent capable of causing a decrease of at least 25%, 50%, 75%, 90%, 95%, 99%, or more in tumor cell survival or proliferation (i.e., a decrease to 75%, 50%, 25%, 10%, 5%, 1% or less of the number of viable cells that would be expected in the absence of the agent) when used in a suitable cell culture system at a concentration equal to or less than approximately 1 mM, 500 μM, 100 μM, 50 μM, 10 μM, 5 μM, 1 μM, 500 nM, 100 nM, 50 nM, 10 nM, 5 nM, 1 nM, 0.5 nM, or 0.1 nM may be screened for, identified, produced, provided, or used. In some embodiments a decrease is between 50% and 75%, between 75% and 90%, between 90% and 95%, between 95% and 100%. A decrease of 100% may be a reduction to background levels or essentially no viable cells or no cell proliferation. In general, any suitable method for assessing tumor cell survival or proliferation may be used.

In some embodiments, genes and/or gene products that regulate HSF1 cancer-related activity are targets of interest for drug development. For example, in some embodiments an inhibitor or activator of a gene product that modulates HSF1 activity in cancer cells is of use to modulate HSF1 cancer-related activity. As but one example, a kinase that phosphorylates HSF1 in cancer cells and thereby increases activity or nuclear localization of HSF1 would be a target of interest for identification and/or development of an inhibitor of the kinase. Such an inhibitor may be useful to inhibit HSF1 in cancer cells, e.g., in cell culture and/or in subjects in need of treatment for cancer. In some embodiments, a screen is performed to identify an inhibitor or activator of a gene product identified as a modulator of HSF1 cancer-related activity. Such a screen may be performed using similar test agents and methods as described above. It will be understood that details of a screen may depend at least in part on the identity of the particular gene product. For example, if the gene product has an enzymatic activity, the screen may utilize a composition comprising the gene product and a substrate of the gene product and may seek to identify test agents that affect utilization or modification of the substrate when present in the composition. Test agents identified as inhibitors or activators of gene products that modulate HSF1 cancer-related activity may be confirmed as modulators of HSF1 cancer-related activity and/or may be tested in an in vitro or in vivo tumor model.

In some aspects, methods of identifying candidate therapeutic agents, e.g., candidate anti-tumor agents are provided. In some embodiments an inhibitor of HSF1 cancer-related activity is a candidate anti-tumor agent. For example, an agent that has been assessed, e.g., by a method described herein, and determined to modulate, e.g., inhibit, HSF1 cancer-related activity, may be considered a candidate therapeutic agent, e.g., a candidate anti-tumor agent. A candidate anti-tumor agent that has been assessed in an ex vivo or in vivo tumor model and has been determined to inhibit tumor cell survival or proliferation or to inhibit tumor development, maintenance, growth, invasion, metastasis, resistance to chemotherapy, recurrence, or otherwise shown a useful anti-tumor effect may be considered an anti-tumor agent. An anti-tumor agent may be tested in a clinical trial in a population of subjects in need of treatment for cancer to confirm its therapeutic utility or further define subject characteristics or tumor characteristics that correlate with (e.g., are predictive of) efficacy or to identify particularly effective agents, combinations, doses, etc. In some embodiments, methods disclosed herein may identify agents that increase HSF1 expression or activity. Agents that increase HSF1 activity may find use as, e.g., cell protective agents (e.g., for neuroprotection, cardioprotection, etc.), longevity-increasing agents, anti-aging agents, etc. For example, increasing HSF1 activity may be useful in protecting cells subjected to stress due to injury, disease, or exposure to cytotoxic or cell damaging agents or in individuals who have mutations or polymorphisms that result in abnormally low HSF1 functional activity, e.g., under stress conditions.

Wherever relevant herein, a difference between two or more values (e.g., measurements) or groups, or a relationship between two or more variables, may be statistically significant. For example, a difference in, or level of inhibition or reduction of, binding, expression, activity, cell proliferation, cell survival, tumor size, tumor number, tumor growth rate, tumor metastasis, e.g., as compared with a reference or control level, may be statistically significant. As used herein, “statistically significant” may refer to a p-value of less than 0.05 using an appropriate statistical test. One of ordinary skill in the art will be aware of appropriate statistical tests and models for assessing statistical significance, e.g., of differences in measurements, relationships between variables, etc., in a given context. Exemplary tests and models include, e.g., t-test, ANOVA, chi-square test, Wilcoxon rank sum test, log-rank test, Cox proportional hazards model, etc. In some embodiments multiple regression analysis may be used. In some embodiments, a p-value may be less than 0.025. In some embodiments, a p-value may be less than 0.01. In some embodiments a two-sided statistical test is used. In some embodiments, a result or outcome or difference between two or more values is “statistically significant” if it has less than a 5%, less than a 2.5%, or less than a 1% probability of occurring by chance. In some embodiments, a difference between two or more values or a relationship between two or more variables may be statistically significant with a p-value of less than 0.05, less than 0.025, or less than 0.01. In some embodiments, values may be average values obtained from a set of measurements obtained from different individuals, different samples, or different replicates of an experiment. Software packages such as SAS, GraphPad, etc., may be used for performing statistical analysis. It will be understood that any values may be appropriately normalized in some embodiments In some aspects, disclosed herein are a composition, nucleic acid construct, or cell comprising: (a) a first isolated nucleic acid comprising a sequence that encodes HSF1; and (b) a second isolated nucleic acid comprising a sequence that encodes YY1. In some aspects, disclosed herein are a composition, nucleic acid construct, or cell comprising: (a) a first agent that modulates expression or activity of HSF1; and (b) a second agent that modulates expression or activity of YY1. In some embodiments the first agent inhibits expression or activity of HSF1 and the second agent inhibits expression or activity of YY1. In some embodiments the first agent and the second agent comprise nucleic acids. In some embodiments the first agent and the second agent comprise RNAi agents.

In some aspects, disclosed herein is a method of modulating expression of an HSF1-CP gene, the method comprising contacting a cell with a first agent that modulates expression or activity of HSF1 and a second agent that modulates expression or activity of YY1. In some embodiments the first agent inhibits expression or activity of HSF1. In some embodiments the first and second agents inhibit expression or activity of HSF1 and YY1, respectively. In some embodiments the first and second agents are RNAi agents. In some embodiments, modulating expression or activity of HSF1 and YY1 may have additive or synergistic effects on, e.g., cancer cell viability or proliferation. In some embodiments, assessing YY1 expression or activity may be useful in conjunction with an HSF1-based assay or method, e.g., for diagnostic, prognostic, treatment selection or other purposes.

Kits and Systems

In some aspects, the invention provides kits comprising reagents suitable for performing an assay to assess HSF1 expression or HSF1 activation, e.g., for use in a method of the invention. Such kits may contain, e.g., (i) a probe or primer (optionally labeled and/or attached to a support) for detecting, reverse transcribing, and/or amplifying an HSF1 RNA, (e.g, HSF1 mRNA); (ii) a probe or primer for detecting, reverse transcribing, and/or amplifying an RNA (e.g., mRNA) transcribed from an HSF1-regulated gene; (iii) an antibody that binds to an HSF1 polypeptide (e.g., for use in IHC); (iv) one or more control reagents; (v) a detection reagent such as a detectably labeled secondary antibody or a substrate; (vi) one or more control or reference samples that can be used for comparison purposes or to verify that a procedure for detecting HSF1 expression or activation is performed appropriately or is giving accurate results. A control reagent can be used for negative or positive control purposes. A control reagent may be, for example, a probe or primer that does not detect or amplify HSF1 mRNA or an antibody that does not detect HSF1 polypeptide or a purified HSF1 polypeptide or portion thereof(e.g., an HSF1 peptide). A probe, primer, antibody, or other reagent may be attached to a support, e.g., a bead, slide, chip, etc.

In some embodiments, a kit comprises any one or more isolated nucleic acids, nucleic acid constructs, vectors, or cells disclosed herein. In some embodiments a kit comprises reagents suitable for assessing expression of one or more HSF1-CP genes. Such kits may contain, for each of one or more HSF1-CP genes, e.g., (i) a probe or primer (optionally labeled and/or attached to a support) for detecting, reverse transcribing, and/or amplifying an RNA (e.g., mRNA) transcribed from an HSF1-CP gene; (ii) a binding agent, e.g., an antibody, that binds to an HSF1-CP polypeptide (e.g., for use in IHC); (iii) one or more control reagents; (iv) a detection reagent such as a detectably labeled secondary antibody or a substrate; (v) one or more control or reference samples that can be used for comparison purposes or to verify that a procedure for detecting HSF1-CP expression or activity is performed appropriately or is giving accurate results.

In some embodiments a kit comprises probes, primers, binding agents, or other primary detection reagents suitable for detecting multiple HSF1-CP mRNA or polypeptides, wherein the probes, primers, binding agents, or other primary detection reagents are attached to a support, e.g., a bead, slide, chip, etc. In some embodiments the primary detection reagents are arranged in an array format, e.g., in mutually perpendicular rows and columns.

In some embodiments the kit comprises a microarray, e.g., an oligonucleotide microarray. In some embodiments, a kit comprises reagents useful to assess expression of one or more HSF1-CSS, HSF1-CaSig2 gene, HSF1-CaSig3 gene, refined HSF1-CSS, Group A, Group B, Module 1, Module 2, Module 3, Module 4, or Module 5 genes. In some embodiments a kit comprises a nucleic acid construct useful as a reporter of HSF1 activity, e.g., as described above. In some embodiments a kit comprises probes, primers, or binding agents, or other primary detection reagents suitable for measuring at least 5%, 10%, 20%, 30%, 40%, 50%, 60%, 70%, 80%, 90%, 95%, or all of the HSF1-CSS, HSF1-CaSig2, HSF1-CaSig3, refined HSF1-CSS, Group A, Group B, Module 1, Module 2, Module 3, Module 4, or Module 5 genes. In some embodiments at least 50% of probes, primers, binding agents, or other primary detection reagents in a kit are specific for HSF1-CP genes.

Individual kit components may be packaged in separate containers (e.g., tubes, bottles, etc.) The individual component containers may be packaged together in a larger container such as a box for commercial supply. Optionally the kit comprises written material, e.g., instructions, e.g., in a paper or electronic format (e.g., on a computer-readable medium). Instructions may comprise directions for performing the assay and/or for interpreting results, e.g., in regard to tumor classification, diagnosis, prognosis, or treatment-specific prediction. Such material could be provided online.

In some embodiments, the invention provides a system which is adapted or programmed to assess HSF1 expression or HSF1 activation, e.g., for use in a method of the invention. In some embodiments the system may include one or more instruments (e.g., a PCR machine), an automated cell or tissue staining apparatus, an imaging device (i.e., a device that produces an image), and/or one or more computer processors. The system may be programmed with parameters that have been selected or optimized for detection and/or quantification of an HSF1 gene product, e.g., in tumor samples. The system may be adapted to perform the assay on multiple samples in parallel and/or may have appropriate software to analyze samples (e.g., using computer-based image analysis software) and/or provide an interpretation of the result. The system can comprise appropriate input and output devices, e.g., a keyboard, display, etc. In some embodiments, the invention provides a system which is adapted or programmed to assess expression of one or more HSF1-CP genes, e.g., one or more HSF1-CSS, HSF1-CaSig2, HSF1-CaSig3, refined HSF1-CSS, Group A, Module 1, Module 2, Module 3, Module 4, or Module 5 genes. In some embodiments a system classifies a sample based on assessing expression of one or more HSF1-CP genes in the sample. In some embodiments, the invention provides a system which is adapted or programmed to assess binding of HSF1 to regulatory regions of one or more HSF1-CP genes, e.g., one or more HSF1-CSS, HSF1-CaSig2, HSF1-CaSig3, refined HSF1-CSS, Group A, Module 1, Module 2, Module 3, Module 4, or Module 5 genes. In some embodiments a system classifies a sample based on assessing binding of HSF1 to regulatory regions of one one or more HSF1-CP genes in the sample.

In some embodiments, an assay is performed at one or more central testing facilities, which may be specially qualified or accredited (e.g., by a national or international organization which, in some embodiments, is a government agency or organization or a medical or laboratory professional organization) to perform the assay and, optionally, provide a result. A sample can be sent to the laboratory, and a result of the assay, optionally together with an interpretation, is provided to a requesting individual or entity. In some embodiments, determining the level of HSF1 expression or the level of HSF1 activation in a sample obtained from the tumor comprises providing a tumor sample to a testing facility. In some aspects, the invention provides a method comprising: providing to a testing facility (a) a sample obtained from a subject; and (b) instructions to perform an assay to assess the level of HSF1 expression or HSF1 activation (and, optionally, instructions to perform one or more additional assays, e.g., one or more additional assays described herein). In some aspects, the invention provides a method comprising: (a) providing to a testing facility a sample obtained from a subject; and (b) receiving results of an assay of HSF1 expression or HSF1 activation. In some aspects, the invention further provides a method comprising providing, e.g., electronically, a result of such an assay, to a requestor. In some aspects, the invention further provides a method comprising receiving, e.g., electronically, a sample and a request for an assay of HSF1 expression or HSF1 activation, performing such assay, and reporting the result of such assay to a requestor. A result can comprise one or more measurements, scores and/or a narrative description. In some embodiments, a result provided comprises a measurement, score, or image of the sample, with associated diagnostic, prognostic, or treatment-specific predictive information. In some embodiments, a result provided comprises a measurement, score, or image of the sample, without associated diagnostic, prognostic, or treatment-specific predictive information. The invention contemplates that an assay may be performed at a testing facility which is remote from the site where the sample is obtained from a subject (e.g., at least 1 kilometer away). It is contemplated that samples and/or results may be transmitted to one or more different entities, which may carry out one or more steps of an assay or a method of the invention or transmit or receive results thereof. All such activities are within the scope of various embodiments of the invention.

### EXEMPLIFICATION

### Materials and Methods Used in Examples 1-8

Study Design and Population

The Nurses' Health Study (NHS) is a prospective cohort study initiated in 1976 (40, 41). 121,700 female US-registered nurses between the ages of 30-55 completed a questionnaire on factors relevant to women's health with follow-up biennial questionnaires used to update exposure information and ascertain non-fatal incident diseases (40). The follow-up rate was greater than 90% through 1996. Participants who developed breast cancer were identified through the biennial questionnaires and permission was obtained for a review of the medical record. The diagnosis of cancer was confirmed by chart review in 99% participants who self-reported the development of breast cancer. Tumor size, existence of metastatic disease, histologic subtype and invasive or in situ status were recorded from the medical record. This information was used to assign a clinical stage to the patients using the parameters listed in the legend of Table 1. In cases of deceased participants, death certificates and medical records were obtained to ascertain information relevant to the study. Use of this information and associated pathology materials for the study reported here was approved by the Human Subjects Committee at Brigham and Women's Hospital in Boston, Mass.

Tissue Microarray Construction

The NHS breast cancer tissue block collection and tissue microarray (TMA) assembly have been described previously (40, 41). Formalin fixed paraffin-embedded tissue blocks were collected from breast cancers that developed within a follow-up period of 20 years spanning 1976 to 1996. Samples were successfully obtained from 3,752 of the 5,620 participants that were eligible for block collection. The diagnosis, tumor type, and histologic grade were confirmed by review of Hematoxylin and eosin (H&E) stained sections. A total of 23 TMA blocks were constructed at the Dana Farber/Harvard Cancer Center Tissue Microarray Core Facility in Boston from 3,093 primary tumors and lymph nodes with metastatic disease derived from 2,897 study participants. For this study, tissue was available from 21TMAs including samples from 2656 individuals.

Paraffin blocks were also obtained from the archives of Brigham and Women's Hospital (BWH) in accordance with the regulations for excess tissue use stipulated by the BWH institutional review board. Twenty-four blocks from individual patients were used to construct an additional tissue microarray from normal breast tissue derived from breast reduction mammoplasty procedures. Normal breast epithelial lobules were identified on H&E stained sections and three 0.6 mm cores were taken and transferred into a recipient paraffin block at the Dana Farber/Harvard Cancer Center Tissue Microarray Core Facility. Epithelium from 16 lobules could be identified in the sections used for this study. Additional whole tissue sections were made from paraffin blocks of invasive ductal carcinoma or ductal carcinoma in situ.

Lung, colon, and prostate tissue studied was also formalin-fixed paraffin-embedded human biopsy material.

Immunohistochemistry of Tissues

Paraffin sections of human and mouse tissues and tissue microarrays were stained with a rat monoclonal antibody cocktail to HSF1 (Thermo Scientific RT-629-PABX).

According to the manufacturer's data sheet, this antibody preparation contains a combination of monoclonal antibodies obtained from hybridoma clones 4B4, 10H4, and 10H8, generated using recombinant mouse HSF1 protein (amino acids 1-503) as an immunogen, and reported to recognize an epitope within amino acids 288-439. Deparaffinized sections were blocked with 3% H2O2, antigen retrieval was performed using a pressure cooker with Dako citrate buffer (pH 6.0) at 120° C.+/−2° C., 15+/−5 PSI, slides were blocked with 3% normal rabbit serum and primary HSF1 antibody (1:2000) was incubated at room temperature for 40 minutes. Application of the primary antibodies was followed by 30 minute incubation with Dako Labeled Polymer-HRP anti-rat IgG as a secondary antibody, and visualized with 3,3′-diaminobenzidine (DAB) as a chromogen (Dako Envision+ System). Mayer-hematoxylin was used for counterstaining.

Immunostained sections were reviewed by light microscopy and scored visually with a value assigned to each individual core. Scoring was based on a semi-quantitative review of staining intensity with 0 indicating no nuclear staining, 1 indicating low level nuclear staining and 2 indicating strong nuclear staining for HSF1. The immunostained sections were evaluated independently by two pathologists (SS and TAI) who were blinded to the survival outcomes of the participants and scores given by the other pathologist. Scoring averages were determined per case from values assigned to all evaluable cores from the two independent readings. If diagnostic tissue was absent or if the staining was uninterpretable for all three cores, the case status was recorded as missing. The kappa value was used to measure inter-observer variability among the two pathologist reviews. The kappa statistic was 0.92 for the scoring of HSF1-positive versus negative tumors and 0.84 for the scoring of HSF1-negative, HSF1-low, versus HSF1-high tumors. Cases with no detectable HSF1 or only cytoplasmic immunoreactivity are referred to as HSF1-negative tumors and cases with low or high nuclear HSF1 are referred to as HSF1-positive tumors unless indicated otherwise. The ER, PR and HER2 status of each case was determined as previously described (42). HSF1 wild-type and null mice as a source of tissue for immunostaining controls were a kind gift from Ivor Benjamin (3).

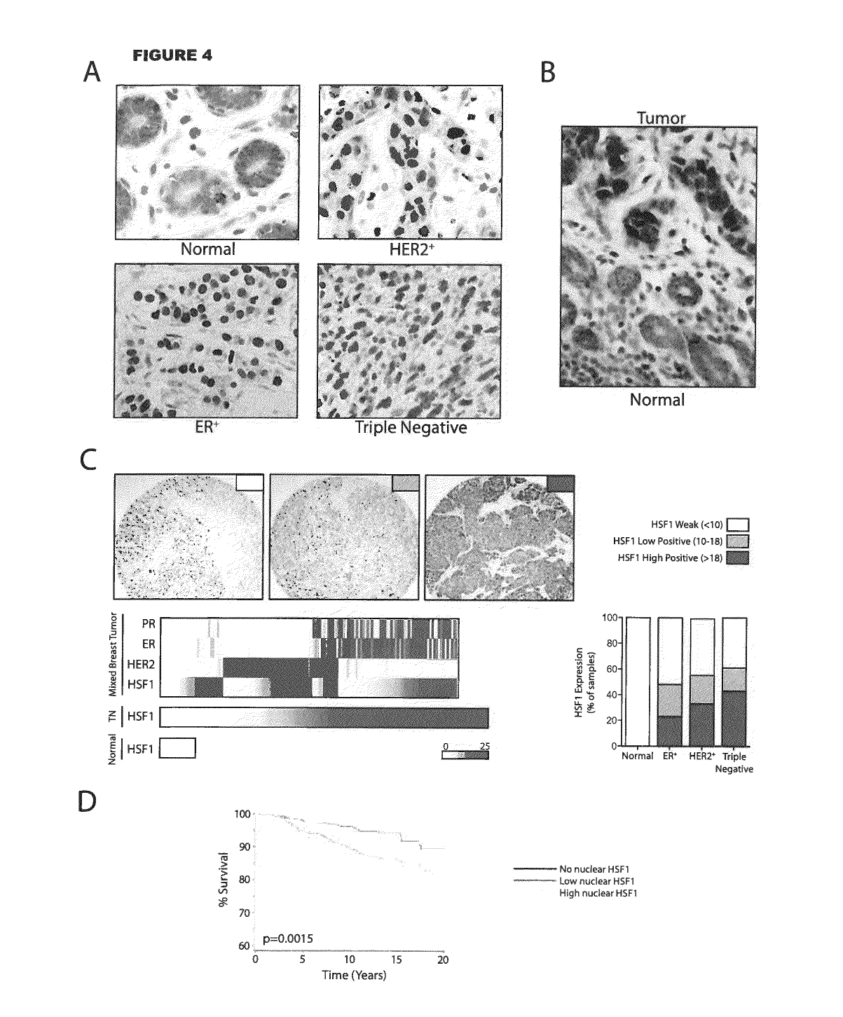

In the analysis depicted in FIGS. 4C and 4D and described in Example 6, scoring was performed as follows: Scoring was based on a 0 to 5 scale for percent of cells that exhibited staining (0 being no staining, 1 being <20% of cells staining, 2 being 20%-40% of cells staining, 3 being 40%-60% of cells staining, 4 being 60%-80% of cells staining, 5 being 80%-100% of cells staining) and a 0 to 5 score for intensity. The percent score and intensity score were then multiplied to get a total score between 0 and 25, thus the overall score ranged from 0-25. Tumors with a score greater than 18 were assigned to the HSF1 high positive group; tumors with a score between 10 and 18 (inclusive) were assigned to the HSF1 low positive group; tumors with a score below 10 were assigned to the HSF1 weak group.

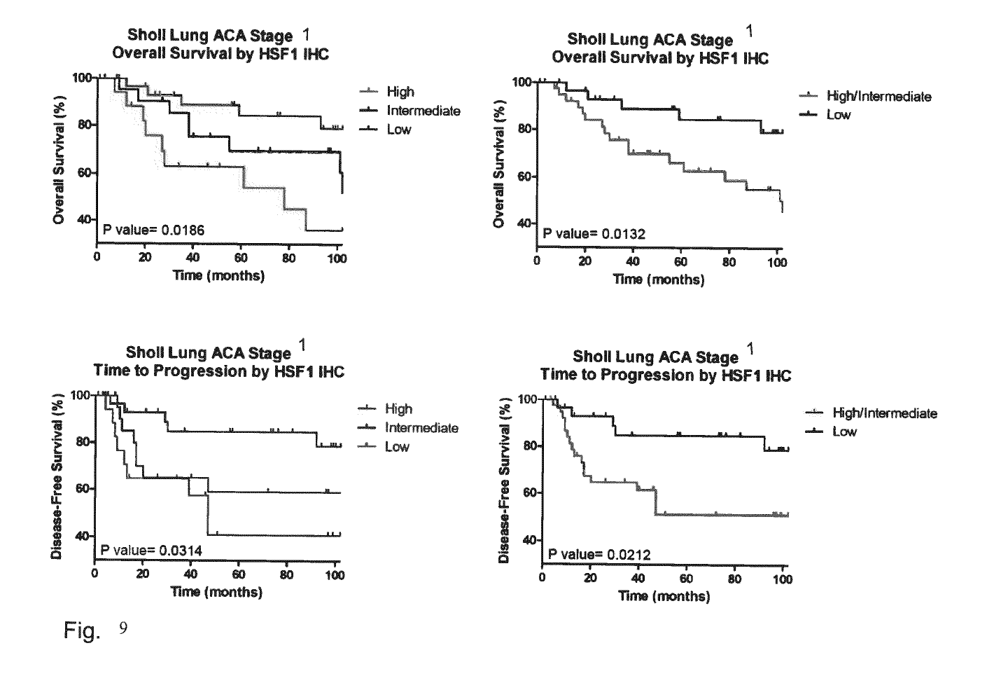

In the analysis described in Example 8 and depicted in FIG. 9, scoring was based on a 0 to 5 scale for percent of cells that exhibited staining (0 being no staining, 1 being <20% of cells staining, 2 being 20%-40% of cells staining, 3 being 40%-60% of cells staining, 4 being 60%-80% of cells staining, 5 being 80%-100% of cells staining) and a 0 to 5 score for intensity. The percent score and intensity score were then multiplied to get a total score between 0 and 25, thus the overall score ranged from 0-25. Tumors with a score greater than or equal to 20 were assigned to the HSF1 high group; the HSF1 intermediate group had a score of 10-20; and the HSF1 low group had scores <10.

Immunoblotting

Tissue blot IMB-130a from Imgenex Corp (San Diego, Calif.) was blocked with 5% non-fat dry milk in IX PBS (pH 7.4) and washed with IX PBS (pH 7.4) containing 0.1% Tween 20. Primary antibodies were applied in IX PBS (pH 7.4)+0.5% non-fat dry milk for 1 hour at room temperature. Peroxidase-conjugated secondary antibodies were applied at room temperature for 1 hour and the signal was visualized by incubation with a chemiluminescent substrate (Pico-West, Thermo-Fisher). Tissues lysates from HSF1 wild-type and null mice were made from freshly harvested organs that were immediately frozen in liquid nitrogen, and subsequently extracted in cold lysis buffer (100 mM NaCl, 30 mM Tris-HCl (pH 7.6), 1% NP-40, 1 mM EDTA, 1 mM sodium orthovanadate, 30 mM sodium fluoride, and a complete protease inhibitor cocktail tablet (Roche Diagnostics)). Protein concentrations were determined using a BCA reagent (Pierce Biochemical) and proteins were separated on NuPAGE® Novex gels and transferred to Immun-Blot® PVDF membrane (Bio-Rad).

Selection Criteria for Outcome Analysis

This study included women with either ductal carcinoma in situ or invasive breast carcinoma that were diagnosed between 1976, after the completion of the baseline initial questionnaire, and 1996. Inclusion in the study (n=2656) required that tissue from the primary breast lesion was available for TMA construction and that outcome data was also available. Kaplan-Meier analysis and multivariate analysis were performed with data from participants with invasive breast cancer at diagnosis. Participants were excluded from outcome analysis if they had in situ carcinoma only (n=408), stage 1V breast cancer at the time of diagnosis (n=50) or HSF1-status could not be evaluated due to missing cores (n=357). Hence, outcome analysis was performed on 1,841 women. Expression of HSF1 was also analyzed in 200 cases of ductal carcinoma in situ which were not included in outcome analysis.

Covariates Evaluated in the Analysis

The medical record and supplemental questionnaires were used to garner information on the breast tumor and treatments including year of diagnosis, stage, radiation, chemotherapy and hormonal treatments. Histological grade was determined by centralized pathology review as described previously (41). Covariates considered in the multivariate model were based on both statistical significance and clinical significance. They included age at diagnosis, date of diagnosis, estrogen receptor status, disease stage, tumor grade, radiation treatment, chemotherapy and hormonal treatment.

Statistical Analysis

HSF1-positive (including HSF1-high and HSF-low) and HSF1-negative tumors were compared according to tumor characteristics and treatment variables by the chi-square test or Wilcoxon rank sum test, as appropriate. The survival endpoint was death from breast cancer. Deaths from any other causes were censored. Therefore, all mention of survival and mortality refer only to breast cancer-specific survival and mortality. Survival curves were estimated by the Kaplan-Meier method and statistical significance was assessed with the log-rank test. Cox proportional hazards regression models were used to evaluate the relationship between HSF1 status and breast cancer-specific mortality after adjusting for covariates. All analyses of the NHS data were run with SAS version 9.1 statistical software. Survival of patients from Van de Vivjer et al. (17) was analyzed by Kaplan-Meier methods and statistical significance was assessed with the log-rank test using GraphPad Prism 5. All statistical tests were two-sided and a P value of <0.05 was considered statistically significant.

### Materials and Methods Used in Examples 9-14

Cell Culture Methods.

HME, HMLER and MCF10A cells were cultured in MEGM medium supplemented as specified by the manufacturer (Lonza). BPE and BPLER cells were cultured in WIT-I and WIT-T medium, respectively, in accordance with recommendations by the manufacturer (Stemgent). The HME, BPE, HMLER and BPLER cells are available from the Ince laboratory upon request. BT474, H441, H838, H1703, HCC38, HCC1954, HCT15, HT29, SKBR3, SW620 and ZR75-1 cells were cultured in RPMI-1640 medium supplemented with 10% fetal bovine serum. BT20, MDA-MB-231, MCF7 and T47D cells were cultured in Dulbecco's modified Eagle's medium supplemented with 10% fetal bovine serum. All established cell lines were from A.T.C.C.

ChIP-Seq and ChIP-PCR.

ChIP-qPCR and ChIP-Seq experiments were performed as described previously (Lee et al., 2006), with modifications and analysis methods detailed in Supplemental Experimental Procedures.

Gene Expression.

Lentiviral shRNA sequences, viral production and transduction of cells have been described previously (Dai et al., 2007). Gene expression analysis was performed as described in Supplemental Experimental Procedures using an Affymetrix Gene Chip HT Human Genome U133 96-Array Plate. Data were analyzed using previously described methods (Ince et al., 2007). All microarray raw data were deposited in a public database (NCBI Gene Expression Omnibus). For ChIP-PCR, HSF1 was depleted using siRNA as described in Supplemental Experimental Procedures.

Immunohistochemistry of Tissues.

Paraffin sections of tissue microarrays were stained using a rat HSF1 monoclonal antibody cocktail (Thermo Scientific, RT-629-PABX) as detailed in Supplemental Experimental Procedures.

The Nurses' Health Study Analysis Design and Population, Exclusion Criteria and Statistical Analysis.

The Nurses' Health Study (NHS) is a prospective cohort study initiated in 1976 (Hu et al., 2011; Tamimi et al., 2008). For design and study population, exclusion criteria and statistical analysis, see above.

Correlation of Gene Expression with Outcome.

The “HSF1-CaSig” was generated from the 456 genes that were bound in BPLER cells by HSF1 near their transcription start sites (bound from −8 kb to +2 kb of the TSS). Table T4C lists the HSF1-CaSig genes. The HSF1-CaSig2 was generated from the genes found in Modules 1 and 2 of our gene-gene correlation analysis (FIG. 4B). Genes within Module 1 showed strong positive correlation with the expression of HSF1 mRNA itself, and Module 2 was positively correlated with Module 1. Table T4E lists the HSF1-CaSig2 genes. (Note: The modules were based on Affymetrix arrays, in which there is typically more than 1 probe per gene. Probes for a given gene usually behave similarly and clustered together. However, this was not always the case. In generating the HSF1-CaSig2, genes for which more probes fell into Modules 3-5 than into Modules 1-2 were excluded). The HSF1-CaSig3 was derived using three training datasets (Hou et al., 2010; Jorissen et al., 2009; Pawitan et al., 2005). We used genes that were (1) bound by HSF1 in our high malignancy model cell line (BPLER): 891 genes or (2) used to assemble our correlation matrix: two of the three cell lines with most robust HSF1 activation (BT20, NCIH838, SKBR3)—which was 1042 genes. The union of (1) and (2) comes to a set of 1543 unique genes. Briefly, the 300 genes from this set that were most positively correlated with poor outcome and the 150 genes from this set that were most negatively correlated (by t-test statistic) with poor outcome were identified in each dataset. Genes present in at least two of three datasets in each group were assembled in the final HSF1-CaSig3 gene signature. Table T4F lists the HSF1-CaSig3 genes. The first 163 genes listed in Table T4F (ABCA7-ZNF453) were positively associated with poor outcome. The last 44 genes listed in Table T4F (AFF2-ZBTB20) were negatively associated with poor outcome.

We used all breast cancer datasets with reported clinical outcome available in the Oncomine database (Rhodes et al., 2007) containing at least 70 tumors, excluding several datasets based on older microarray platforms that were missing many currently annotated genes. This left 10 high-quality datasets, the majority of which contained more than 150 tumors (Table T5). We stratified each dataset into two groups of tumors based on high (highest 25%) and low (lowest 75%) average expression of the gene or gene signature being queried. For analysis of the MammaPrint and the HSF1-CaSig3 gene signature, the subset of genes positively correlating with poor outcome was positively weighted and the subset of genes negatively correlating with poor outcome was negatively weighted, as described previously (van 't Veer et al., 2002; van de Vijver et al., 2002). Data for the three versions of the HSF1-CaSig for KM analysis were retrieved from Oncomine (Rhodes et al., 2007).

All data for comparisons with random signatures were obtained from NCBI GEO and KM analysis was repeated. (The VandeVijver and TCGA datasets were not on an Affymetrix platform and were excluded from this analysis.) If CEL files were available, Affymetrix microarrays were processed with RMA using Bioconductor; otherwise, preprocessed expression matrices were obtained from NCBI GEO or author web sites. Monte Carlo cross validation was applied to contrast HSF1-CaSig signatures with random signatures of genes of the same number. Random sets of signatures containing the same number of probesets as each HSF1 signature were generated for each dataset with a particular emphasis on U133A probesets (present on both U133A and U1133 Plus 2.0 arrays). The 10,000 random signatures were processed in the same manner as the original signature, sorting samples by increasing mean expression of each mean-centered probeset. Cancer samples, partitioned into the high and low HSF1-CaSig as before, were then analyzed for survival with the log-rank test, producing 10,000 test statistics. Median p values were calculated across a tumor subtype and Monte Carlo cross validation was applied.

Statistical Analysis.

Correlation of gene expression with location of HSF1 occupancy was performed using a two-tailed Fisher's Exact Test. Statistical methods for ChIP-Seq analysis and the Nurses' Health Study outcome data analysis are detailed in Supplemental Experimental Procedures. Kaplan-Meier analysis was used to compare outcome events and p-values were generated using the logrank test. For all other data, mean+/−standard deviation is reported and statistical significance between means was determined using a two-tailed t test.

Gene-Gene Correlation Analysis.

Correlation values of HSF1-bound genes were determined by using the UCLA Gene Expression Tool (genome.ucla.edu/projects/UGET) to query gene expression profile data collected in Celsius, a data warehousing system that aggregates Affymetrix CEL files and associated metadata. Nearly 12,000 Affymetrix HG-U133 Plus 2.0 human gene expression profiles, predominantly representing neoplasms of highly diverse human origin, were interrogated.

Supplemental Experimental Procedures for Examples 9-14

ChIP Antibodies.

For ChIP-Seq, HSF1 antibody (Santa Cruz, sc-9144) and normal rabbit IgG (Santa Cruz, sc-2027) were used. For ChIP-qPCR, HSF1 antibody (Santa Cruz, sc-9144) and, as a control, a second HSF1 antibody (Thermo Scientific, RT-629-PABX), were used. Similar results were obtained and RT-629-PABX antibody data are reported. Additionally, (RNA polymerase II CTD repeat YSPTSPS antibody [4H8](Abcam, ab5408) and normal rabbit IgG (Santa Cruz, sc-2027) were used, as indicated.

ChIP-Seq and ChIP-PCR.

For ChIP-Seq, 5×10⁷ cells were used for each immunoprecipitation. For heat-shock, cells were transferred to a 42′C (5% CO₂) incubator for 1 hr. ChIP and ChIP-Seq experiments were performed as described previously (Lee et al., 2006) with several modifications (Novershtern et al., 2011). In place of RIPA buffer, immunoprecipitations were washed sequentially with buffer B (20 mM Tris-HCl, pH 8.0, 150 mM NaCl, 2 mM EDTA, pH 8.0, 0.1% SDS and 1.0% Triton X-100), buffer C (20 mM Tris-HCl, pH 8.0, 500 mM NaCl, 2 mM EDTA, pH 8.0, 0.1% SDS and 1.0% Triton X-100), buffer D (10 mM Tris-HCl, pH 8.0, 250 mM LiCl, 1 mM EDTA, pH 8.0, 1.0% Na-Deoxycholate and 1.0% IGEPAL CA-630), and buffer TE (10 mM Tris-HCl, pH 8.0, 1 mM EDTA, pH 8.0). Preparation of the ChIP-Seq DNA library and deep sequencing using an Illumina Solexa genome analyzer were performed as described previously (Yu et al., 2009).

Images acquired from the Illumina sequencer were processed through the bundled Illumina image extraction pipeline. ChIP-Seq reads were aligned to HG18 using ELAND software (Illumina). Identification of enriched genomic regions was performed as described previously (Guenther et al., 2008). Briefly, each ChIP-Seq read (a maximum of two repeat reads were allowed) was extended 100 bp to approximate the middle of the sequenced fragment. The extended fragments were subsequently allocated to 25 bp bins across the genome. Read density for each bin was calculated and enriched bins were identified by comparison to a Poisson background model using a p-value threshold of 10⁻¹². The minimum ChIP-seq read density required to meet this threshold for each dataset is indicated in Table T1. Enriched bins within 200 bp were combined to form enriched regions. Enriched regions less than 100 bp were removed. Because of the non-random nature of background reads, enriched bins and regions were also required to have an eight-fold greater ChIP-seq density versus a nonspecific control IgG immunoprecipitation performed under identical conditions. All RefSeq genes that were within 8 kb of enriched regions were considered to be enriched genes. A summary of the experiments is provided in Table T1. The raw data will be or have been deposited in a public database (NCBI Gene Expression Omnibus).

The unions of all HSF1 enriched regions identified by ChIP-Seq in each sample were merged to identify a global set of regions. Short reads overlapping these regions were quantified using HTSeq-count (http://www-huber.embl.de/users/anders/HTSeq/doc/count.html). The counts matrix was median-normalized using the total number of mapped reads. After adding 1 pseudocount, counts were log 2-normalized and analyzed by principal components as implemented by the MADE4 program in Bioconductor (Culhane et al., 2005).

For ChIP-qPCR, 5×10⁶ cells were used for each immunoprecipitation. The protocol was modified as described above. RT² SYBR Green qPCR Mastermix (SABiosciences) was used with the indicated oligo pairs (Table T7) on a 7700 ABI Detection System.

Preparation of human breast and colon tumors for ChIP-seq was performed using 300 mg of cryopreserved material. Frozen tumor tissue was retrieved from the Brigham and Women's Hospital (BWH) Tissue Bank in accordance with the regulations for excess tissue use stipulated by the BWH institutional review board. Frozen sections for immunohistochemistry were prepared using a cryostat from adjacent tissue. Frozen samples were processed for ChIP-Seq using a tissue pulverizer, and this material was subsequently suspended in PBS and passed serially through needles of increasing gauge. This suspension was then fixed for 10 minutes and the pellet was processed as described above.

Gene Expression Analysis.

Lentiviral shRNA sequences, viral production and transduction of cells have been described previously (Dai et al., 2007). RNA was purified following extraction with TRIzol reagent (Invitrogen, #15596-026), 60 hours after viral infection. Protein lysates of concurrent infections were prepared in TNES buffer consisting of 50 mM Tris, pH 7.4; NP-40 1%; EDTA 2 mM; NaCl 200 mM plus protease inhibitor cocktail (Roche Diagnostics, Cat#11836153001). Protein concentration was measured by BCA assay (Thermo Fisher Scientific 23227) and 15 μg total protein/lane was analyzed by SDS-PAGE and immunoblotting using rat monoclonal anti-HSF1 antibody cocktail (Ab4, Thermo Scientific, 1:1000 dilution) and Actin Monoclonal Antibody (mAbGEa; clone DM1A, Thermo Scientific, 1:1,000). Because prolonged depletion of HSF1 is toxic to malignant cells (Dai et al., 2007), we analyzed mRNA expression early, before HSF1 knockdown was complete and cell viability was grossly impaired. Thus, results likely underestimate the effects of HSF1 on gene expression in malignant cells. For gene expression after heat-shock, cells were transferred to a 42° C. (5% CO₂) incubator for 1 hr and allowed to recover for 30 minutes in a 37° C. (5% CO₂) incubator before RNA extraction. Gene expression analysis was performed using an Affymetrix GeneChip HT Human Genome U133 96-Array Plate and data were analyzed using previously described methods (Ince et al., 2007). All microarray raw data were deposited in a public database (NCBI Gene Expression Omnibus).

For evaluating the effects of HSF1 knockdown on the expression of target genes, HSF1 was depleted using siRNA (Dharmacon, Lafayette, Colo.): M012109-01 siGenome SMART pool, Human HSF1 (target sequences:

(SEQ ID NO. 4)
UAGCCUGCCUGGACAAGAA;
CCACUUGGAUGCUAUGGAC;
(SEQ ID NO. 5)
GAGUGAAGACAUAAAGAUC;
AGAGAGACGACACGGAGUU).

siGLO RISC-Free siRNA (D-001600-01) and siGENOME Non-Targeting siRNA #5 (D-001210-05) were used as controls. Cells were transfected using Lipofectamine™ RNAiMAX Transfection Reagent (Invitrogen, #13778) and were harvested in Trizol (Invitrogen, #15596-026). RNA was purified using Direct-zol™ RNA MiniPrep (Zymo Research, Irving, Calif.). Quantitative PCR to evaluate mRNA levels was performed as described above using RT² SYBR Green qPCR Mastermix (SABiosciences) and primer assay pairs (SABiosciences; Valencia, Calif.) on a 7700 ABI Detection System.

Gene-Gene Correlation Analysis.

Correlation values of HSF1-bound genes were determined using the UCLA Gene Expression Tool (genome.ucla.edu/projects/UGET) to query gene expression profile data collected in Celsius, a data warehousing system that aggregates Affymetrix CEL files and associated metadata. Nearly 12,000 Affymetrix HG-U133 Plus 2.0 human gene expression profiles, predominantly representing neoplasms of highly diverse human origin, were interrogated. A pair-wise correlation matrix was built by assessing genes bound in at least two of the three cell lines with most robust HSF1 activation (BT20, NCIH838, SKBR3). This generated 1042 genes. The final map as displayed contains 709 unique genes, with genes required to have an absolute value of the correlation coefficient >0.3 (|a|>0.3) with at least 100 other genes. Data was ordered using hierarchical clustering (correlation centered, average linkage).

Xenografts.

5×10⁶ HMLER and BPLER cells in a 50/50 mix of PBS/Matrigel were inoculated subcutaneously in the right inguinal region of each mouse using a 27 g needle. Tumors were removed, and fixed in 10% formalin. Following standard tissue processing, 5 μM sections were cut and immunostained as described below.

Immunohistochemistry of Tissues and Scoring.

Paraffin blocks of human tumor and normal tissue were obtained from the archives of BWH in accordance with the regulations for excess tissue use stipulated by the BWH institutional review board. Tissue microarrays were purchased from Pantomics (Richmond, Calif.) for carcinoma of the breast (BRC501, BRC1502), cervix (CXC1501), colon (COC1503), lung (LUC1501), pancreas (PAC481) and prostate (PRC1961). Whole sections of 40 meningioma specimens were retrieved from the archives of BWH. A TMA of triple negative breast cancer cases was kindly provided by Dr. Andrea Richardson (BWH). Normal tissue cores on the TMAs and adjacent normal tissues in the whole sections were used to evaluate expression of HSF1 in non-neoplastic tissues.

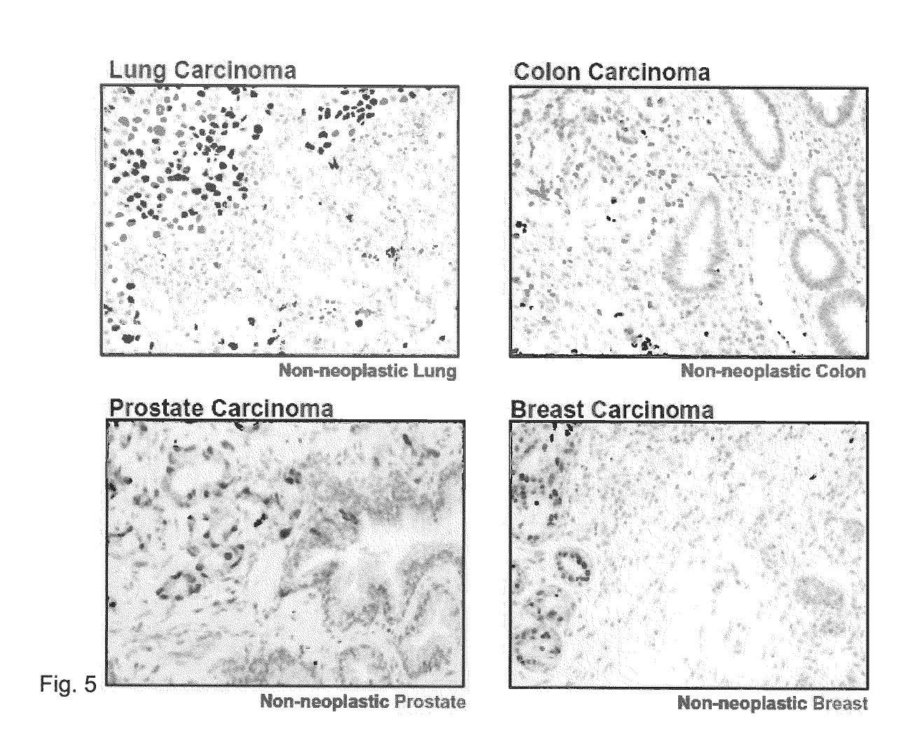

Formalin-fixed, paraffin-embedded (FFPE) sections were first deparaffinized. Frozen sections were first post-fixed in 10% formalin. FFPE or fixed-frozen sections were blocked with 3% H2O2 and antigen retrieval was performed using a pressure cooker with Dako citrate buffer (pH 6.0) at 120° C.+/−2° C., 15+/−5 PSI. Slides were blocked using 3% normal rabbit serum, primary HSF1 antibody (1:2000) was applied at room temperature for 40 minutes, followed by a 30 minute incubation with Dako Labeled Polymer-HRP anti-rat IgG as a secondary antibody. Visualization was achieved with 3,3′-diaminobenzidine (DAB) as a chromogen (Dako Envision+ System). Counterstaining was performed with Mayer-hematoxylin. Immunostained sections were scored independently by two pathologists (SS and TAI) using light microscopy. HSF1 immunostains of FFPE tumor sections were scored using a 0 to 25 scale in FIG. 5. The percent of tumor cells staining with HSF1 was quantified as (0)=0%; (1+)=1-20%; (2+)=21-40%; (3+)=41-60%; (4+)=61-80%; (5+)=81-100%. The intensity of nuclear staining was quantified 0 to 5+ relative to negative normal cells. The total HSF1 score was derived by multiplying the percent score with the intensity score. Three tiers of HSF1 staining were defined based on total combined scores of less than 10 (Weak HSF1); 10-18 (Low-Positive HSF1), >18 (High-Positive HSF1).

Immunofluorescence.

Immunofluorescence was performed using 1:250 dilution of rat monoclonal anti-HSF1-antibody cocktail (Ab4, Thermo Scientific, 1:1000 dilution), 1:100 dilution of rabbit polyclonal anti-p53 (Santa Cruz, #sc-6243) and with fluorescence labeled secondary antibodies. The slides were then reviewed by standard fluorescence microscope.

TABLE T7
Oligonucleotides used in this study.

| | | SEQ |
|---|---|---|
| | | ID |
| NAME | SEQUENCE | NO. |
| AANAT/Ube2O-qPCR-F | GAGCCGTAGGTCCCTTCTTT | 6 |
| AANAT/Ube2O-qPCR-R | CTCAGGAACCTTCCAGACCA | 7 |
| CKS2-qPCR-F | ACCGACTACGTCATCACCAA | 8 |
| CKS2-qPCR-R | GTGGAAAGTTCCAGGACACG | 9 |
| Jarid2-qPCR-F | TTGGTTGCGCTTTTAGCTTT | 10 |
| Jarid2-qPCR-R | ACCCCAAGTCACAGAGATGG | 11 |
| Maf1/Sharpin-qPCR-F | TTTGCCCACAAATGGACAC | 12 |
| Maf1/Sharpin-qPCR-R | CCCAAAGACCAGCTCTAACG | 13 |
| Pgk1-qPCR-F | TCTCGCACATTCTTCACGTC | 14 |
| Pgk1-qPCR-R | AGGAACCTTCCCGACTTAGG | 15 |
| RBM23-qPCR-F | TTGGGGTTTCTCACCAGTTC | 16 |
| RBM23-qPCR-R | CTGCAGTGCTGCTTTTCTTG | 17 |
| HspA6-qPCR-F | GATCTGCCCGAACCTTCTC | 18 |
| HspA6-qPCR-R | AACTTTCGCGAACCTTTCC | 19 |
| HspA8-qPCR-F | CCACCCTGCCTCTTATACCC | 20 |
| HspA8-qPCR-R | GGCTTGTGATTGGGTCTTGT | 21 |
| HSPD1-qPCR-F | CGGCCGGCTTAGTCTAGTT | 22 |
| HSPD1-qPCR-R | ATTTGACCCTTGAGCCGTAG | 23 |
| BCL10-qPCR-F | TGAGTCATATGGGTGTGCTG | 24 |
| BCL10-qPCR-R | TCCCCTTAGCACAGAAGTGA | 25 |
| Ncor2-qPCR-F | GGGTGGAATTACAGCCTCAG | 26 |
| Ncor2-qPCR-R | TCCTGTAGCTCCCACACCTC | 27 |
| DHFR1-qPCR-F | ACCTGGTCGGCTGCACCT | 28 |
| DHFR1-qPCR-R | TTGCCCTGCCATGTCTCG | 29 |
| Intergenic-qPCR-F | ATGTCAGGCCCATGAACGAT | 30 |
| Intergenic-qPCR-R | GCATTCATGGAGTCCAGGCTTT | 33 |

References cited in Supplemental Experimental Procedures for Examples 9-14
Bild, A. H., Yao, G., Chang, J. T., Wang, Q., Potti, A., Chasse, D., Joshi, M. B, Harpole, D., Lancaster, J. M., Berchuck, A., et al. (2006). Oncogenic pathway signatures in human cancers as a guide to targeted therapies. Nature 439, 353-357.
Culhane, A. C., Thioulouse, J., Perriere, G., and Higgins, D. G. (2005). MADE4: an R package for multivariate analysis of gene expression data. Bioinformatics 21, 2789-2790.
Dai, C., Whitesell, L., Rogers, A. B., and Lindquist, S. (2007). Heat shock factor 1 is a powerful multifaceted modifier of carcinogenesis. Cell 130, 1005-1018.
Desmedt, C., Piette, F., Loi, S., Wang, Y., Lallemand, F., Haibe-Kains, B., Viale, G., Delorenzi, M., Zhang, Y., d'Assignies, M. S., et al. (2007). Strong time dependence of the 76-gene prognostic signature for node-negative breast cancer patients in the TRANSBIG multicenter independent validation series. Clin Cancer Res 13, 3207-3214.
Guenther, M. G., Lawton, L. N., Rozovskaia, T., Frampton, G. M., Levine, S. S., Volkert, T. L., Croce, C. M., Nakamura, T., Canaani, E., and Young, R. A. (2008). Aberrant chromatin at genes encoding stem cell regulators in human mixed-lineage leukemia. Genes Dev 22, 3403-3408.
Hou, J., Aerts, J., den Hamer, B., van Ijcken, W., den Bakker, M., Riegman, P., van der Leest, C., van der Spek, P., Foekens, J. A., Hoogsteden, H. C., et al. (2010). Gene expression-based classification of non-small cell lung carcinomas and survival prediction. PLoS One 5, e10312.
Hu, R., Dawood, S., Holmes, M. D., Collins, L. C., Schnitt, S. J., Cole, K., Marotti, J. D., Hankinson, S. E., Colditz, G. A., and Tamimi, R. M. (2011). Androgen receptor expression and breast cancer survival in postmenopausal women. Clin Cancer Res 17, 1867-1874.
Ince, T. A., Richardson, A. L., Bell, G. W., Saitoh, M., Godar, S., Karnoub, A. E., Iglehart, J. D., and Weinberg, R. A. (2007). Transformation of different human breast epithelial cell types leads to distinct tumor phenotypes. Cancer Cell 12, 160-170.
Jorissen, R. N., Gibbs, P., Christie, M., Prakash, S., Lipton, L., Desai, J., Kerr, D., Aaltonen, L. A., Arango, D., Kruhoffer, M., et al. (2009). Metastasis-Associated Gene Expression Changes Predict Poor Outcomes in Patients with Dukes Stage B and C Colorectal Cancer. Clin Cancer Res 15, 7642-7651.
Lee, T. I., Johnstone, S. E., and Young, R. A. (2006). Chromatin immunoprecipitation and microarray-based analysis of protein location. Nat Protoc 1, 729-748.
Loi, S., Haibe-Kains, B., Desmedt, C., Lallemand, F., Tutt, A. M., Gillet, C., Ellis, P., Harris, A., Bergh, J., Foekens, J. A., et al. (2007). Definition of clinically distinct molecular subtypes in estrogen receptor-positive breast carcinomas through genomic grade. J Clin Oncol 25, 1239-1246.
Loi, S., Haibe-Kains, B., Desmedt, C., Wirapati, P., Lallemand, F., Tutt, A. M., Gillet, C., Ellis, P., Ryder, K., Reid, J. F., et al. (2008). Predicting prognosis using molecular profiling in estrogen receptor-positive breast cancer treated with tamoxifen. BMC Genomics 9, 239.
Minn, A. J., Gupta, G. P., Siegel, P. M., Bos, P. D., Shu, W., Girl, D. D., Viale, A., Olshen, A. B., Gerald, W. L., and Massague, J. (2005). Genes that mediate breast cancer metastasis to lung. Nature 436, 518-524.
Novershtern, N., Subramanian, A., Lawton, L. N., Mak, R. H., Haining, W. N., McConkey, M. E., Habib, N., Yosef, N., Chang, C. Y., Shay, T., et al. (2011). Densely interconnected transcriptional circuits control cell states in human hematopoiesis. Cell 144, 296-309.
Pawitan, Y., Bjohle, J., Amler, L., Borg, A. L., Egyhazi, S., Hall, P., Han, X., Holmberg, L., Huang, F., Klaar, S., et al. (2005). Gene expression profiling spares early breast cancer patients from adjuvant therapy: derived and validated in two population-based cohorts. Breast Cancer Res 7, R953-964.
Schmidt, M., Bohm, D., von Tome, C., Steiner, E., Puhl, A., Pilch, H., Lehr, H. A., Hengstler, J. G., Kolbl, H., and Gehrmann, M. (2008). The humoral immune system has a key prognostic impact in node-negative breast cancer. Cancer Res 68, 5405-5413.
Smith, J. J., Deane, N. G., Wu, F., Merchant, N. B., Zhang, B., Jiang, A., Lu, P., Johnson, J. C., Schmidt, C., Bailey, C. E., et al. (2010). Experimentally derived metastasis gene expression profile predicts recurrence and death in patients with colon cancer. Gastroenterology 138, 958-968.
Tamimi, R. M., Baer, H. J., Marotti, J., Galan, M., Galaburda, L., Fu, Y., Deitz, A. C., Connolly, J. L., Schnitt, S. J., Colditz, G. A., et al. (2008). Comparison of molecular phenotypes of ductal carcinoma in situ and invasive breast cancer. Breast Cancer Res 10, R67.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Wang, Y., Klijn, J. G., Zhang, Y., Sieuwerts, A. M., Look, M. P., Yang, F., Talantov, D., Timmermans, M., Meijer-van Gelder, M. E., Yu, J., et al. (2005). Gene-expression profiles to predict distant metastasis of lymph-node-negative primary breast cancer. Lancet 365, 671-679.
Yu, M., Riva, L., Xie, H., Schindler, Y., Moran, T. B., Cheng, Y., Yu, D., Hardison, R., Weiss, M. J., Orkin, S. H., et al. (2009). Insights into GATA-1-mediated gene activation versus repression via genome-wide chromatin occupancy analysis. Mol Cell 36, 682-695.

### Example 1

### Characterization of HSF1 Antibody and HSF1 Expression in Breast Cancer and Various Other Cancer Types

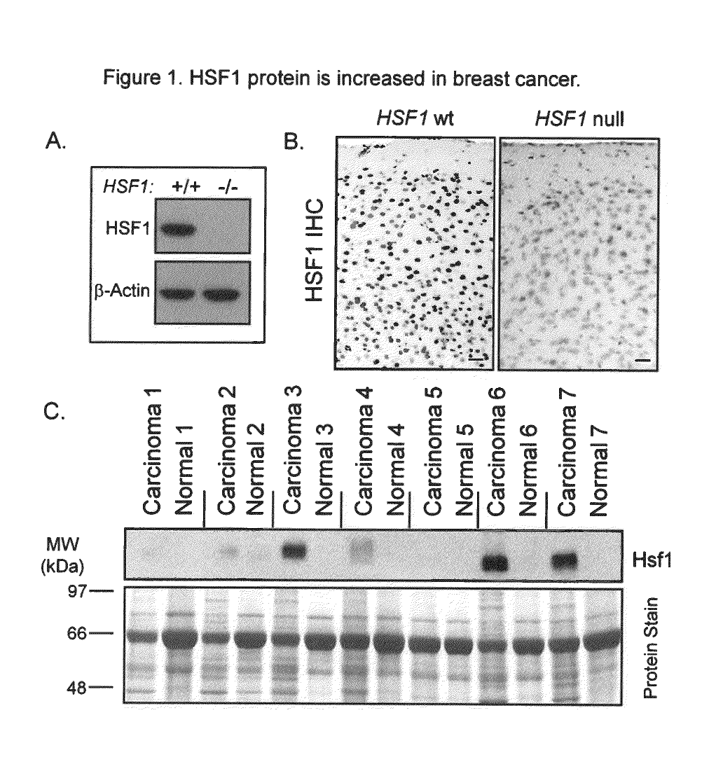

To facilitate our studies of HSF1, we verified the specificity of a commercially-available HSF1 antibody cocktail on samples from HSF1 wild-type and null mice. A strong immunoreactive band of the expected size for HSF1 was present in wild-type lysates but was absent in lysates null for HSF1 (FIG. 1A). Strong nuclear staining was observed by immunohistochemistry (IHC) in wild-type mouse tissues but not in corresponding tissues from HSF1 null mice (FIG. 1B) validating this antibody cocktail for IHC applications.

We examined the expression of HSF1 in invasive carcinoma and matched normal adjacent breast tissue from seven patients by immunoblot (FIG. 1C). More HSF1 was present in the tumors than the matched controls in all cases. Interestingly, there was a strong HSF1 band in three of seven samples obtained from the tumors and moderate to weak bands in the remaining tumors. The variation observed in this pilot study indicated that human breast tumors express HSF1 at different amounts, and encouraged us to examine whether the amount of HSF1 protein expression correlates with prognosis.

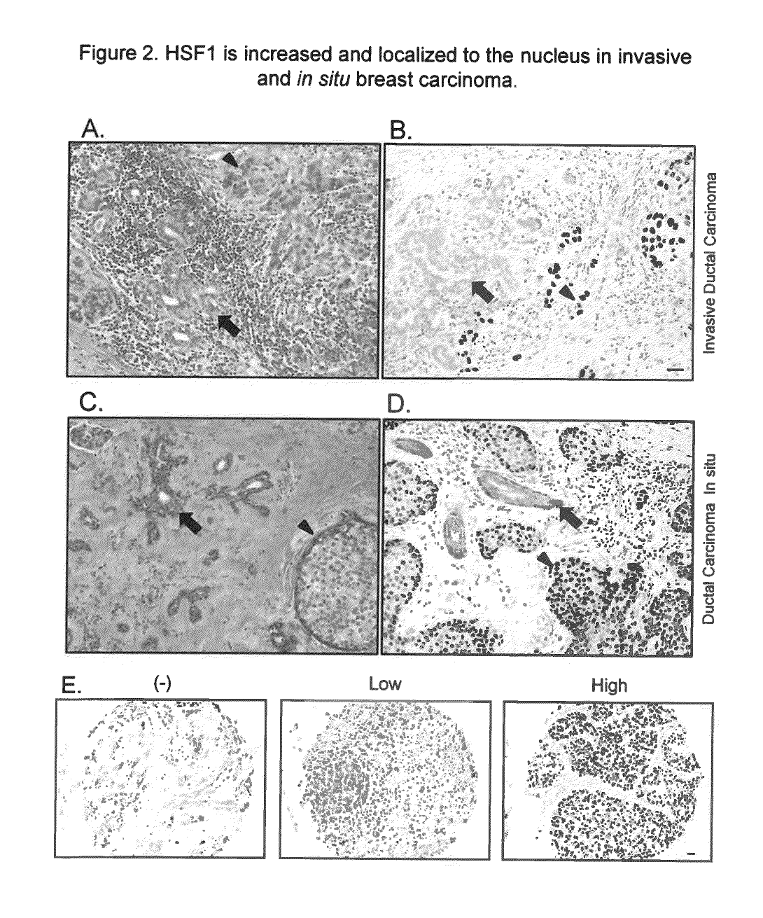

As a transcription factor HSF1 is active only in the nucleus. Hence, we examined the localization and expression levels of HSF1 in tumor cells versus normal cells by IHC in a small panel of breast carcinoma tissue sections. A striking difference between malignant cells and the adjacent normal breast epithelium was apparent (FIGS. 2A, 2B). While no nuclear HSF1 was detectable in nearly all cases in normal breast epithelium (n=16), there was nuclear staining in the majority of breast tumors. In samples of normal breast and in the tumors lacking nuclear HSF1, there was occasionally a weak cytoplasmic signal. The increase in HSF1 levels and its shift from the cytoplasm in normal cells into the nucleus in invasive tumors supported the premise that HSF1 is activated in the malignant state.

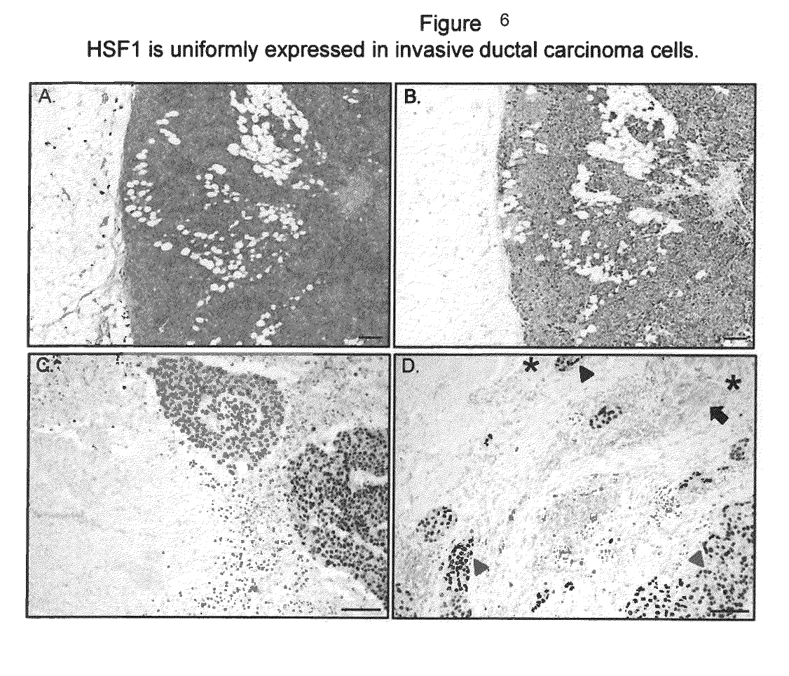

In 20 HSF1-positive tumors, there was widespread uniform expression of HSF1 throughout the tumor cell nuclei. The uniform intensity of HSF1 expression is important to contrast with the variable patterns seen with most prognostic markers that are surveyed in human tumor sections with IHC. HSF1 staining was not stronger in tumor cells at the center of the tumor versus those at the stromal interface (FIG. 6A-B), or in regions of necrosis where microenvironmental stress was likely to be severe (FIG. 6C). Staining intensity was also not dependent on the distance from stromal desmoplasia, inflammation or microvasculature (FIG. 6C-D). Without wishing to be bound by any theory, these observations suggest that increases in HSF1 in tumor cells are not principally due to external microenvironmental stress but more commonly result from internal, cell autonomous factors.

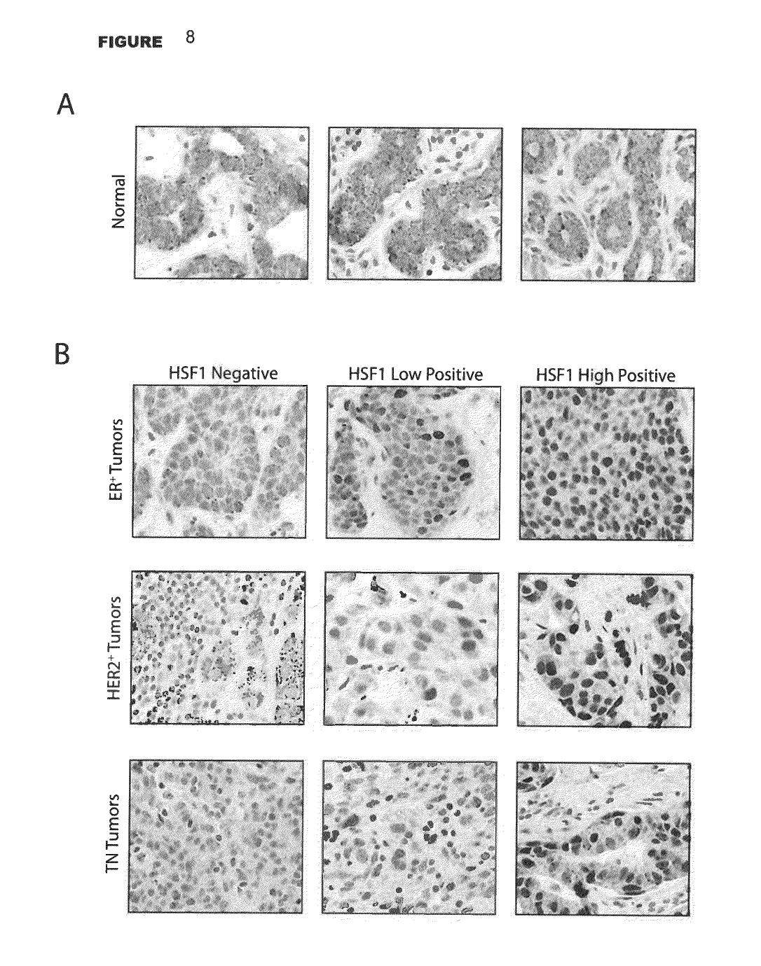

We also monitored HSF1 localization and levels of expression by immunohistochemistry (IHC) in a set of 301 clinical cases of invasive ductal carcinoma. The tumors were also characterized for expression of conventional breast cancer biomarkers, including estrogen receptor (ER), progesterone receptor (PR) and HER2. In total, 67 ER+ and/or PR+ tumors, 54 HER2+ tumors, and 180 triple negative (TN) tumors were evaluated along with 16 normal mammary tissue samples. In samples of normal breast tissue, HSF1 was rarely present in the nucleus (FIGS. 4A and 8). In stark contrast, HSF1 staining was dramatically elevated in many breast tumors and the signal was most often localized to the nucleus (FIGS. 4A, 4B and 8). Interestingly, higher levels of HSF1 staining were seen in HER2+ and TN tumors (FIG. 4C), which are breast cancer subtypes associated with more malignant behavior and worse outcome.

The findings in ten in situ carcinomas were similar to those in invasive cancer. In the majority of ductal carcinoma in situ (DCIS) cases, there was increased nuclear HSF1 compared to neighboring normal breast epithelium (FIG. 2C, 2D). The levels of HSF1 were also uniform in the DCIS cells (i.e., staining intensity was similar among the DCIS cells). These findings suggest that HSF1 expression is elevated during the in situ stage of malignant transformation and prior to invasion as well as subsequently.

We also examined HSF1 expression and localization in a range of other tumor types including lung, colon, and prostate adenocarcinomas using IHC. Increased HSF1 expression and increased nuclear HSF1 were seen in the neoplastic tissue in each of these tumor types (FIG. 5). Elevated HSF1 expression and nuclear localization were also observed in cervical cancer and malignant peripheral nerve sheath tumors (data not shown).

### Example 2

### Nuclear HSF1 is Highest in High-Grade Breast Cancer and is Associated with Advanced Clinical Stage at Diagnosis

We next performed an in-depth analysis of HSF1 protein expression in a large breast cancer cohort. 1,841 invasive breast cancer cases from the Nurses' Health Study (NHS) were evaluated for HSF1 localization and expression (FIG. 2E). 404 (21.9%) were negative for nuclear HSF1 and 1437 had detectable nuclear HSF1 (78.1%) with 882 (47.9%) demonstrating low and 555 (30.2%) high HSF1. Levels of HSF1 expression differed by histological-grade (P<0.0001). 40.5% of well-differentiated low-grade carcinomas were HSF1-negative and only 14.4% showed high nuclear HSF1 (Table 1). Conversely, in poorly-differentiated high-grade cancers, only 13.0% were HSF1-negative and 48.1% showed high HSF1 expression. Levels of HSF1 also differed by clinical parameters. Compared with HSF1-negative tumors, those with nuclear HSF1 expression were more likely to be diagnosed at a more advanced clinical stage (P<0.0001) (Table 1). Also, compared with HSF1-negative tumors, high-HSF1 tumors were more likely to be ER-negative (P<0.0001), HER2-positive (P=0.0003) and triple-negative (P=0.0084) supporting an association between HSF1 expression and a more malignant phenotype.

TABLE 1
Means and frequencies of participants' characteristics by HSF1-status
(N = 1841), Nurses' Health Study (1976-1996).

| Characteristic | None | | Low | | High | |
|---|---|---|---|---|---|---|
| N (%) | 404 | (21.9) | 882 | (47.9) | 555 | (30.2) |
| Age at diagnosis, | 57.8 | (404) | 56.8 | (882) | 57.6 | (555) |
| mean (N), yr | | | | | | |
| Menopausal status | | | | | | |
| at diagnosis, N* (%) | | | | | | |
| Premenopausal | 74 | (18.6) | 219 | (25.3) | 109 | (20.2) |
| Postmenopausal | 325 | (81.5) | 648 | (74.7) | 432 | (79.9) |
| ER status, N* (%) | | | | | | |
| Positive | 334 | (82.7) | 702 | (79.4) | 412 | (71.2) |
| Negative | 70 | (17.3) | 182 | (20.6) | 167 | (28.8) |
| HER2 status, N* (%) | | | | | | |
| Positive | 23 | (5.8) | 95 | (0.7) | 81 | (14.1) |
| Negative | 375 | (94.2) | 794 | (89.3) | 494 | (85.9) |
| Triple-negative | | | | | | |
| tumors, N* (%) | | | | | | |
| Yes | 49 | (12.2) | 122 | (13.7) | 108 | (18.7) |
| No | 353 | (87.8) | 768 | (86.3) | 471 | (81.4) |
| Nodal | | | | | | |
| involvement, N (%) | | | | | | |
| None | 290 | (71.8) | 590 | (66.9) | 324 | (58.4) |
| 1-3 | 72 | (17.8) | 166 | (18.8) | 134 | (24.1) |
| 4-9 | 26 | (6.4) | 78 | (8.8) | 55 | (9.9) |
| ≧10 | 16 | (4.0) | 48 | (5.4) | 42 | (7.6) |
| Tumor size | | | | | | |
| (cm), N (%) | | | | | | |
| ≦2 | 301 | (74.5) | 589 | (66.8) | 295 | (53.2) |
| >2 | 103 | (25 5) | 293 | (33.2) | 260 | (46.9) |
| Histological | | | | | | |
| grade, N* (%) | | | | | | |
| I (low) | 143 | (35.8) | 159 | (18.2) | 51 | (9.3) |
| II (intermediate) | 199 | (49.8) | 543 | (62.1) | 284 | (51.7) |
| III (high) | 58 | (14.5) | 173 | (19.8) | 214 | (39.0) |
| Stage†, N (%) | | | | | | |
| I | 239 | (59.2) | 452 | (51.3) | 217 | (39.1) |
| II | 114 | (28.2) | 283 | (32.1) | 225 | (40.5) |
| III | 51 | (12.6) | 147 | (16.7) | 113 | (20.4) |
| Chemotherapy, | | | | | | |
| N* (%) | | | | | | |
| Yes | 101 | (33.2) | 263 | (41.9) | 217 | (50.6) |
| No | 203 | (66.8) | 365 | (58.1) | 212 | (49.4) |
| Hormone | | | | | | |
| treatment, N* (%) | | | | | | |
| Yes | 207 | (68.8) | 415 | (66.3) | 280 | (66 0) |
| No | 94 | (31.2) | 211 | (33.7) | 144 | (34.0) |
| Radiation | | | | | | |
| treatment, N* (%) | | | | | | |
| Yes | 136 | (44.4) | 275 | (43.7) | 185 | (43.3) |
| No | 170 | (55.6) | 354 | (56.3) | 242 | (56.7) |

*N doesn't add to total because of missing information.
†Stage I = tumor size <= 2 cm and no nodal involvement; II = tumor size <= 2 cm & 1-3 nodes or 2-4 cm & 0-3 nodes or 4+ cm & 0 nodes; III = tumor size <= 2cm & 4+ nodes or 2-4 cm & 4+ nodes or >4 cm & 1+ nodes.

### Example 3

### HSF1 Accumulates in the Nuclei of In Situ Carcinomas

Nuclear HSF1 was detected in 84.5% of the DCIS cases. The frequency and levels of HSF1 expression were similar between DCIS and invasive cancer, confirming our earlier observations on a smaller number of tumor sections. No statistically significant association was found between HSF1 expression and DCIS nuclear grade, however (Table S1). Our limited sample size of DCIS cases (n=200) may have limited the power to detect such an association. Nonetheless, these observations highlight that HSF1 is activated before malignant cells gain the ability to invade across the basement membrane.

TABLE S1
Frequency of HSF1 expression in DCIS according to
tumor grade, Nurses' Health Study (1976 to 1996).
Number of cases and (%). Chi-square analysis.

| HSF1 Expression | None | Low | MM | P-value |
|---|---|---|---|---|
| DCIS | | | | 0.4907 |
| DCIS, low nuclear grade | 4 (22.2) | 11 (61.1) | 3 (16.7) | |
| DCIS, intermediate grade | 16 (16.3) | 54 (56.8) | 25 (26.3) | |
| DCIS, high nuclear grade | 11 (12.6) | 46 (52.9) | 30 (34.5) | |

Chi square analysis of HSF1-negative, HSF1-low and HSF1-high: P = 0.4907.

### Example 4

### HSF1 Expression is Associated with Reduced Survival in Breast Cancer

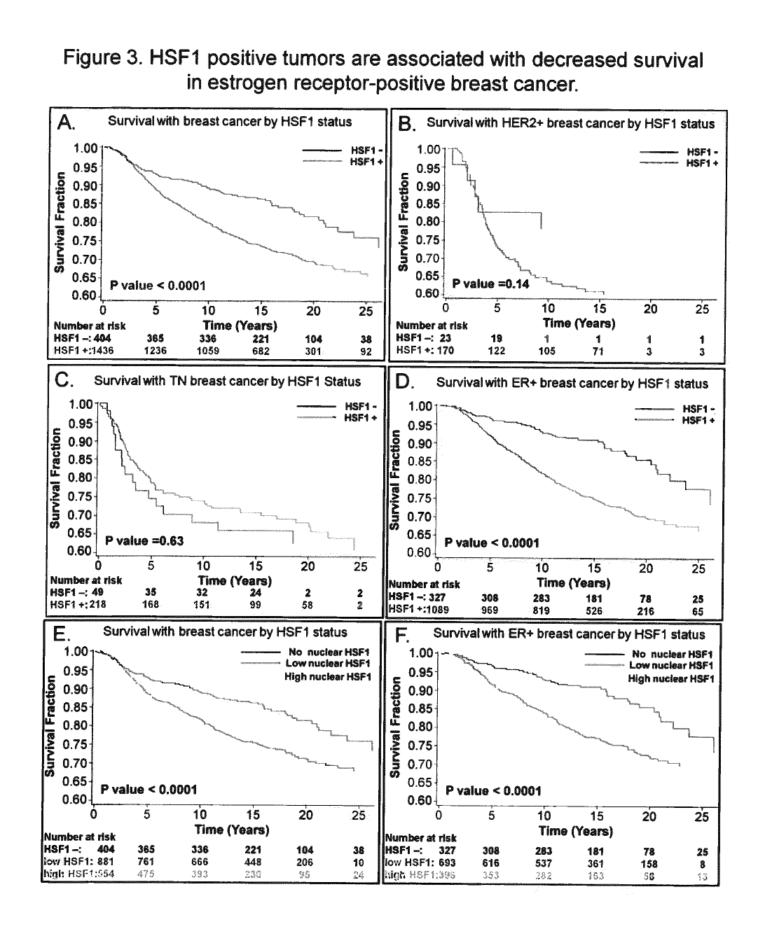

We next investigated the relationship between HSF1 expression and breast cancer survival. A total of 1841 women met inclusion criteria such as the absence of metastases at the time of diagnosis. Median follow-up time was 14.9 years. Kaplan-Meier curves show that women with HSF1-positive tumors had worse survival relative to women with HSF1-negative tumors (P<0.0001) (FIG. 3A). While a suggestive association was observed in the HER2-positive population (P=0.14) (FIG. 3B), no significant association was seen in triple-negative cases (P=0.63) (FIG. 3C). Because of the relatively small number of cases in the ER-negative groups, the study is likely underpowered to observe an effect in those populations. However, in women with ER-positive tumors, a strong association was observed between HSF1-positive tumors and worse outcome (P<0.0001) (FIG. 3D).

We also examined survival considering HSF1-status in three categories: HSF1-negative, HSF1-low and HSF1-high groups. Survival decreased as HSF1 levels increased from none to low and still further to high (P<0.0001) suggesting a dose-dependent association between HSF1 and survival outcomes (FIG. 3E). Dose-dependence was not seen for HER2-positive (P=0.22) and triple-negative populations (P=0.74) but was present in patients with ER-positive tumors (P<0.0001) (FIG. 3F).

### Example 5

### In Multivariate Models HSF1 is a Significant Independent Predictor of Worse Outcome

To account for the effects of all variables considered on the relationship between HSF1 levels and survival, we assessed this relationship using several multivariate models. Across all cases, adjusting for age (model 1, Table 2), HSF1 positive tumors were associated with a 74% increase in breast cancer mortality (Table 2; Hazards Ratio (HR) 1.74, 95% Confidence Interval (CI), 1.35-2.25; P value<0.0001) relative to HSF1-negative tumors. After adjusting for age, ER-status, date of diagnosis, stage, grade, and treatment variables (radiotherapy, chemotherapy, endocrine therapy) (model 2, Table 2), HSF1 positive tumors were associated with a 50% increase in breast cancer mortality (Table 2; HR 1.50, 95% CI, 1.15-1.95; P value=0.0026). HSF1-low and HSF1-high tumors were associated with 45% (P=0.008) and 62% (P=0.001) increases in mortality, respectively (Table 3). Similar results were seen in the ER-positive population with HSF1-positive tumors associated with 86% increased mortality (Table 2; HR, 1.86; 95% CI, 1.34-2.59; P value=0.0002). Among the HSF1-positive tumors, HSF1-low and HSF1-high tumors were associated with 75% and 110%/o increases in mortality, respectively (Table 3).

74% (n=700) of the ER-positive patients received hormonal therapy. In this group, there was a significant association between HSF1-positive tumors and increased mortality (Table 2; HR, 2.20; 95% CI, 1.19-4.05; P value=0.0115). In women with ER-positive tumors who did not receive hormonal therapy (26%, n=247), the magnitude of the association was similar (Table 2; HR, 2.01; 95% CI, 0.69-5.88; P value=0.2002) but the study may have been underpowered to detect a significant association in this group. The data may suggest that HSF1 can contribute to tamoxifen resistance, an effect that may be evaluated further in follow-up studies prospectively in a uniformly-treated population.

HSF1 was also associated with worse clinical outcomes in patients with HER2-positive breast cancer. We observed that 88.4% of HER2-positive invasive tumors were HSF1-positive and 40.7% had high levels of HSF1, the greatest percentage of any molecular subtype. In Kaplan-Meier analysis, a suggestive association between HSF1-status and survival in patients with HER2-positive tumors was observed (FIG. 3B). In multivariate model 2, accounting for additional covariates, the strength of association increased and was statistically significant (Table 2; HR 2.87; 95% CI, 1.12-7.39; P value=0.0288). No association was observed between HSF1-status and survival among triple-negative patients (P=0.64) in multivariate models.

TABLE 2
Multivariate analysis of breast cancer-
specific mortality by HSF1-status.

| | N | | Hazard Ratio (95% CI*) | |
|---|---|---|---|---|
| | | End- | HSF1- | HSF1- |
| Models | Cases | points | negative | positive |
| All cases: | | | | |
| Model¹ | 1841 | 483 | 1.00 | 1.74 (1.35-2.25) |
| Model² | 1841 | 463 | 1.00 | 1.50 (1.15-1.95) |
| ER-positive cases: | | | | |
| Model¹ | 1418 | 327 | 1.00 | 2.21 (1.60-3.06) |
| Model³ | 1416 | 327 | 1.00 | 1.86 (1.34-2.59) |
| ER-negative cases; | | | | |
| Model¹ | 403 | 135 | 1.00 | 0.86 (0.56-1.32) |
| Model³ | 403 | 135 | 1.00 | 0.88 (0.570-1.39) |
| HER2-positive cases: | | | | |
| Model¹ | 194 | 71 | 1.00 | 2.06 (0.83-5.12) |
| Model² | 194 | 71 | 1.00 | 2.87 (1.12-7.39) |
| HER2-negative cases: | | | | |
| Model¹ | 1621 | 388 | 1.00 | 1.61 (1.23-2.11) |
| Model² | 1621 | 386 | 1.00 | 1.37 (1.04-1.80) |
| Triple-negative cases: | | | | |
| Model¹ | 268 | 86 | 1.00 | 0.88 (0.52-1.50) |
| Model³ | 268 | 86 | 1.00 | 0.88 (0.50-1.53) |
| ER-positive with | | | | |
| hormone therapy cases: | | | | |
| Model¹ | 700 | 122 | 1.00 | 2.77 (1.52-5.02) |
| Model⁴ | 700 | 122 | 1.00 | 2.20 (1.19-4.05) |
| ER-positive without | | | | |
| hormone therapy cases: | | | | |
| Model¹ | 247 | 38 | 1.00 | 3.22 (114-9.10) |
| Model⁴ | 247 | 38 | 1.00 | 2.01 (0.69-5.83) |

*CI denotes confidence interval,
Model¹: Adjust for age at diagnosis (years).
Model²: Adjust for age at diagnosis (years), estrogen receptor status (positive, negative), date of diagnosis (months), disease stage (I, II, III), grade (I, II, III), radiation treatment (yes, no, missing), chemotherapy and hormonal treatment (no/no, yes/no, no/yes, yes/yes, missing).
Model³: Adjust for age at diagnosis (years), date of diagnosis (months), disease stage (I, II, III), grade (I, II, III), radiation treatment (yes, no, missing), chemotherapy and hormonal treatment (no/no, yes/no, no/yes, yes/yes, missing).
Model⁴: Adjust for age at diagnosis (years), date of diagnosis (months), disease stage (I, II, III), grade (I, II, III), radiation treatment (yes, no, missing) and chemotherapy (yes, no, missing).

TABLE 3
Multivariate analysis of breast cancer-
specific mortality by HSF1-status.

| | N | | | | |
|---|---|---|---|---|---|
| | | End- | Hazard Ratio (95% CI) | | |
| Models | Cases | points | None | Low | High |
| All cases: | | | | | |
| Model¹ | 1841 | 463 | 1.00 | 1.61 (1.23-2.11) | 1.97 (1.49-2.62) |
| Model² | 1841 | 483 | 1.00 | 1.45 (1.10-1.91) | 1.02 (1.21-2.17) |
| ER- | | | | | |
| positive | | | | | |
| cases: | | | | | |
| Model¹ | 1416 | 327 | 1.00 | 1.98 (1.41-2.78) | 2.66 (1.87-3.79) |
| Model³ | 1418 | 327 | 1.00 | 1.75 (1.25-2.47) | 2.10 (1.45-3.03) |

*CI denotes confidence interval.
Model¹: Adjust for age at diagnosis (years).
Model²: Adjust for age at diagnosis (years), estrogen receptor status (positive, negative), date of diagnosis (months), disease stage (I, II, III), grade (I, II, III), radiation treatment (yes, no, missing), chemotherapy and hormonal treatment (no/no, yes/no, no/yes, yes/yes, missing).
Model³: Adjust for age at diagnosis (years), date of diagnosis (months), disease stage (I, II, III), grade (I, II, III), radiation treatment (yes, no, missing), chemotherapy and hormonal treatment (no/no, yes/no, no/yes, yes/yes, missing).

### Example 6

### HSF1 Activation is an Independent Prognostic Indicator of Poor Outcome in ER+/Lymph Node Negative Breast Tumors

We undertook an analysis of a subset of 947 women in the NHS cohort with ER+/lymph node negative tumors. This population is challenging to manage clinically since it is often unclear which small fraction of the population will experience a recurrence and could therefore benefit from early intervention and more aggressive treatment. Survival was examined by KM analysis considering HSF1-status in three categories: HSF1-negative, HSF1-low and HSF1-high groups. Survival decreased as HSF1 levels increased from none to low and further to high (P=0.0015) suggesting a dose-dependent association between HSF1 activation and survival (FIG. 4D). Multivariate analysis was performed to account for the effects of co-variates including age, date of diagnosis, stage, grade, and treatment variables (radiotherapy, chemotherapy, endocrine therapy). The association remained statistically significant, with the HSF1-positive (low+high cases) tumors associated with a 59% increase in mortality (Table 4), and with high-HSF1 tumors associated with a 98% increase in mortality (Table 5). This analysis demonstrates that even in one of the most challenging breast cancer populations from a prognostic standpoint, HSF1 activation is an independent prognostic indicator of poor outcome.

TABLE 4
Multivariate analysis of breast cancer-
specific mortality by HSF1-status.

| Models | | | | |
|---|---|---|---|---|
| ER-positive, node | N | | Hazard Ratio (95% CI*) | |
| negative cases: | Cases | Endpoints | HSF1-negative | HSF1-positive |
| Model¹ | 947 | 142 | 1.00 | 1.89(1.20-2.98) |
| Model² | 947 | 142 | 1.00 | 1.59(1.00-2.53) |

*CI denotes confidence interval.
Model¹: Adjust for age at diagnosis (years).
Model²: Adjust for age at diagnosis (years), date of diagnosis (months), disease stage (I, II, III), grade (I, II, III), radiation treatment (yes, no, missing), chemotherapy and hormonal treatment (no/no, yes/no, no/yes, yes/yes, missing).

TABLE 5
Multivariate analysis of breast cancer-specific mortality by HSF1-status.

| Models | | | | | |
|---|---|---|---|---|---|
| ER-positive, node | N | | Hazard Ratio (95% CI) | | |
| negative cases: | Cases | Endpoints | None | Low | High |
| Model¹ | 947 | 142 | 1.00 | 1.65 (1.02-2.66) | 2.41 (1.45-3.99) |
| Model² | 947 | 142 | 1.00 | 1.42 (0.88-2.31) | 1.98 (1.17-3.33) |

*CI denotes confidence interval.
Model¹: Adjust for age at diagnosis (years).
Model²: Adjust for age at diagnosis (years), date of diagnosis (months), disease stage (I, II, III), grade (I, II, III), radiation treatment (yes, no, missing), chemotherapy and hormonal treatment (no/no, yes/no, no/yes, yes/yes, missing).

### Example 7

### HSF1 mRNA Expression is Associated with Reduced Survival in Breast Cancer

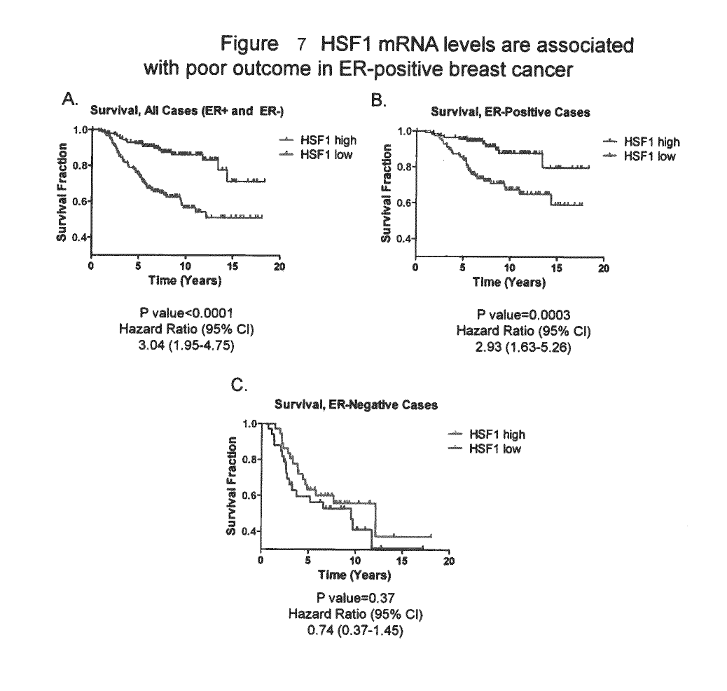

We examined whether the associations between HSF1 protein level and outcome in breast cancer could also be detected using HSF1 mRNA levels. Since mRNA expression profiling data is not available from tumors in the NHS, we used data from the publicly available van de Vijver cohort (17) for this analysis. Consistent with our immunohistochemistry analysis in the NHS sample obtained from the tumors, HSF1 mRNA levels were higher in ER-negative than in ER-positive cancers (P<0.0001). We analyzed survival using two HSF1 categories: HSF1-high and HSF1-low. Kaplan-Meier curves show that women with HSF1-high tumors in the van de Vijver cohort had worse survival relative to women with HSF1-low tumors (FIG. 7A; HR 3.04; 95% CI, 1.95-4.75; P value<0.0001). The difference in survival between women with HSF1-high tumors and HSF1-low tumors was seen in the ER-positive (FIG. 7B; HR 2.93; 95% CI, 1.63-5.26; P value=0.0003) but not in the ER-negative population (FIG. 7C; HR 0.74, 95% CI, 0.37-1.45; P value=0.3736).

### Example 8

### HSF1 Expression is Associated with Reduced Survival in Lung Cancer

We performed IHC for HSF1 protein in tissue samples from a group of 70 stage I lung cancers (Stage I lung adenocarcinomas (T1 N0 M0 or T2 N0 M0)) and examined the relationship between HSF1 expression and overall survival and progression-free survival. Survival was examined by KM analysis considering HSF1-status in three categories: HSF1-low, HSF1-intermediate, and HSF1-high groups. Both overall survival and time to progression decreased as HSF1 levels increased from low to intermediate and further to high, suggesting a dose-dependent association between HSF1 activation and survival (FIG. 9, left panels). The differences were statistically significant (P value=0.0186 for overall survival; P value=0.0314 for time to progression). When HSF1-intermediate and HSF1-high groups were combined, the difference between the HSF1-low and the HSF1-high/intermediate groups were even more evident (FIG. 9, right panels; P value=0.0132 for overall survival; P value=0.0212 for time to progression).

### REFERENCE LIST 1

### Numbering Corresponds to Citations in Examples 1-8 and Detailed Description

1. Rabindran S K, Giorgi G, Clos J, & Wu C (1991) Molecular cloning and expression of a human heat shock factor, HSF1. Proc Natl Acad Sci USA 88(16):6906-6910.
2. Wiederrecht G, Seto D, & Parker C S (1988) Isolation of the gene encoding the S. cerevisiae heat shock transcription factor. Cell 54(6):841-853.
3. Xiao X, et al. (1999) HSF1 is required for extra-embryonic development, postnatal growth and protection during inflammatory responses in mice. EMBO J 18(21):5943-5952.
4. Guertin M J & Lis J T (2010) Chromatin landscape dictates HSF binding to target DNA elements. PLoS Genet 6(9).
5. Page T J, et al. (2006) Genome-wide analysis of human HSF1 signaling reveals a transcriptional program linked to cellular adaptation and survival. Mol Biosyst 2(12):627-639.
6. Dai C, Whitesell L, Rogers A B, & Lindquist S (2007) Heat shock factor 1 is a powerful multifaceted modifier of carcinogenesis. Cell 130(6):1005-1018.
7. Luo J, Solimini N L, & Elledge S J (2009) Principles of cancer therapy: oncogene and non-oncogene addiction. Cell 136(5):823-837.
8. Solimini N L, Luo J, & Elledge S J (2007) Non-oncogene addiction and the stress phenotype of cancer cells. Cell 130(6):986-988.
9. Meng L, Gabai V L, & Sherman M Y (2010) Heat-shock transcription factor HSF1 has a critical role in human epidermal growth factor receptor-2-induced cellular transformation and tumorigenesis. Oncogene 29(37):5204-5213.
10. Min J N, Huang L, Zimonjic D B, Moskophidis D, & Mivechi N F (2007) Selective suppression of lymphomas by functional loss of Hsf1 in a p53-deficient mouse model for spontaneous tumors. Oncogene 26(35):5086-5097.
11. Ciocca D R & Calderwood S K (2005) Heat shock proteins in cancer: diagnostic, prognostic, predictive, and treatment implications. Cell Stress Chaperones 10(2):86-103.
12. Barginear M F, et al. (2008) The heat shock protein 90 chaperone complex: an evolving therapeutic target. Curr Cancer Drug Targets 8(6):522-532.
13. Whitesell L & Lindquist S L (2005) HSP90 and the chaperoning of cancer. Nat Rev Cancer 5(10):761-772.
14. Hoang A T, et al. (2000) A novel association between the human heat shock transcription factor 1 (HSF1) and prostate adenocarcinoma. Am J Pathol 156(3):857-864.
15. Khaleque M A, et al. (2008) Heat shock factor 1 represses estrogen-dependent transcription through association with MTA1. Oncogene 27(13):1886-1893.
16. Khaleque M A, et al. (2005) Induction of heat shock proteins by heregulin beta1 leads to protection from apoptosis and anchorage-independent growth. Oncogene 24(43):6564-6573.
17. van de Vijver M J, et al. (2002) A gene-expression signature as a predictor of survival in breast cancer. N Engl J Med 347(25):1999-2009.
18. Robert F & Pelletier J (2009) Translation initiation: a critical signalling node in cancer. Expert Opin Ther Targets 13(11):1279-1293.
19. Williams B R, et al. (2008) Aneuploidy affects proliferation and spontaneous immortalization in mammalian cells. Science 322(5902):703-709.
20. Scott K L, et al. (2011) Proinvasion metastasis drivers in early-stage melanoma are oncogenes. Cancer Cell 20(1):92-103.
21. Cowen L E & Lindquist S (2005) Hsp90 potentiates the rapid evolution of new traits: drug resistance in diverse fungi. Science 309(5744):2185-2189.
22. Michor F & Polyak K (The origins and implications of intratumor heterogeneity. Cancer Prev Res (Phila) 3(11):1361-1364.
23. Merlo L M, et al. (2010) A comprehensive survey of clonal diversity measures in Barrett's esophagus as biomarkers of progression to esophageal adenocarcinoma. Cancer Prev Res (Phila) 3(11):1388-1397.
24. Higgins M J & Steams V (2009) Understanding resistance to tamoxifen in hormone receptor-positive breast cancer. Clin Chem 55(8):1453-1455.
25. Singh R R, Barnes C J, Talukder A H, Fuqua S A, & Kumar R (2005) Negative regulation of estrogen receptor alpha transactivation functions by LIM domain only 4 protein. Cancer Res 65(22):10594-10601.
26. Manavathi B, Singh K, & Kumar R (2007) MTA family of coregulators in nuclear receptor biology and pathology. Nucl Recept Signal 5:e010.
27. Kumar R, et al. (2002) A naturally occurring MTA1 variant sequesters oestrogen receptor-alpha in the cytoplasm. Nature 418(6898):654-657.
28. Zhao Y H, et al. (2009) Upregulation of lactate dehydrogenase A by ErbB2 through heat shock factor 1 promotes breast cancer cell glycolysis and growth. Oncogene 28(42):3689-3701.
29. Ince T A, et al. (2007) Transformation of different human breast epithelial cell types leads to distinct tumor phenotypes. Cancer Cell 12(2):160-170.
30. Calderwood S K (2010) Heat shock proteins in breast cancer progression—a suitable case for treatment?Int J Hyperthermia 26(7):681-685.
31. de Billy E, Powers M V, Smith J R, & Workman P (2009) Drugging the heat shock factor 1 pathway: exploitation of the critical cancer cell dependence on the guardian of the proteome. Cell Cycle 8(23):3806-3808.
32. Whitesell L & Lindquist S (2009) Inhibiting the transcription factor HSF1 as an anticancer strategy. Expert Opin Ther Targets 13(4):469-478.
33. Mayer I A (2009) Treatment of HER2-positive metastatic breast cancer following initial progression. Clin Breast Cancer 9 Suppl 2:S50-57.
34. Modi S, et al. (2007) Combination of trastuzumab and tanespimycin (17-AAG, KOS-953) is safe and active in trastuzumab-refractory HER-2 overexpressing breast cancer: a phase I dose-escalation study. J Clin Oncol 25(34):5410-5417.
35. Modi S, et al. (2011) HSP90 Inhibition is Effective in Breast Cancer: A Phase 2 Trial of Tanespimycin (17AAG) plus Trastuzumab in Patients with HER2-Positive Metastatic Breast Cancer Progressing on Trastuzumab. Clin Cancer Res 17(15):5132-9.
36. Kamal A, et al. (2003) A high-affinity conformation of Hsp90 confers tumour selectivity on Hsp90 inhibitors. Nature 425(6956):407-410.
37. Ramanathan R K, et al. (2010) Phase I pharmacokinetic and pharmacodynamic study of 17-dimethylaminoethylamino-17-demethoxygeldanamycin, an inhibitor of heat-shock protein 90, in patients with advanced solid tumors. J Clin Oncol 28(9):1520-1526.
38. Trepel J, Mollapour M, Giaccone G, & Neckers L (2010) Targeting the dynamic HSP90 complex in cancer. Nat Rev Cancer 10(8):537-549.
39. Whitesell L, Bagatell R, & Falsey R (2003) The stress response: implications for the clinical development of hsp90 inhibitors. Curr Cancer Drug Targets 3(5):349-358.
40. Hu R, et al. (2011) Androgen receptor expression and breast cancer survival in postmenopausal women. Clin Cancer Res 17(7):1867-1874.
41. Tamimi R M, et al. (2008) Comparison of molecular phenotypes of ductal carcinoma in situ and invasive breast cancer. Breast Cancer Res 10(4):R67.
42. Dawood S, et al. (2011) Defining breast cancer prognosis based on molecular phenotypes: results from a large cohort study. Breast Cancer Res Treat 126:185-92.

### Example 9

### HSF1 is Activated in Highly Tumorigenic Cells

To investigate the HSF1-regulated transcriptional network in cancer and how it relates to the classical heat-shock response, we used a panel of human mammary epithelial cell lines with very different abilities to form tumors and metastasize (Ince et al., 2007). Two types of primary mammary epithelial cells (HMEC and BPEC) were isolated from normal breast tissue derived from the same donor during reductive mammoplasty. These pairs of isogenic cells were established using different culture conditions that are believed to have supported the outgrowth of distinct cell types. The cells were immortalized with hTERT (HME and BPE) and then transformed with an identical set of oncogenes (HMLER and BPLER). The resulting tumorigenic breast cell lines had very different malignant and metastatic potentials (low, HMLER and high, BPLER) supporting the concept that the cell type from which a cancer arises (“cell-of-origin”) can significantly influence its ultimate phenotype (Ince et al., 2007). Despite their initial isogenic nature and transformation by the same oncogenes, the tumor initiating cell frequency in BPLER cells is ˜10⁴ times greater (more tumorigenic) than isogenic HMLER cells derived from the same donor (Ince et al., 2007). While HMLER cells are non-metastatic, the BPLER cells form metastases in lungs from orthotopic and subcutaneous tumors with very high frequency (>75-85%) (Ince et al., 2007). Hence, the panel of immortalized, non-tumorigenic cells (HME and BPE) and their transformed counterparts with low (HMLER) and high (BPLER) malignant potential provided a well-controlled system for simultaneously studying the changes that occur during transformation as well as the molecular differences that drive variation in malignant potential (Ince et al., 2007).

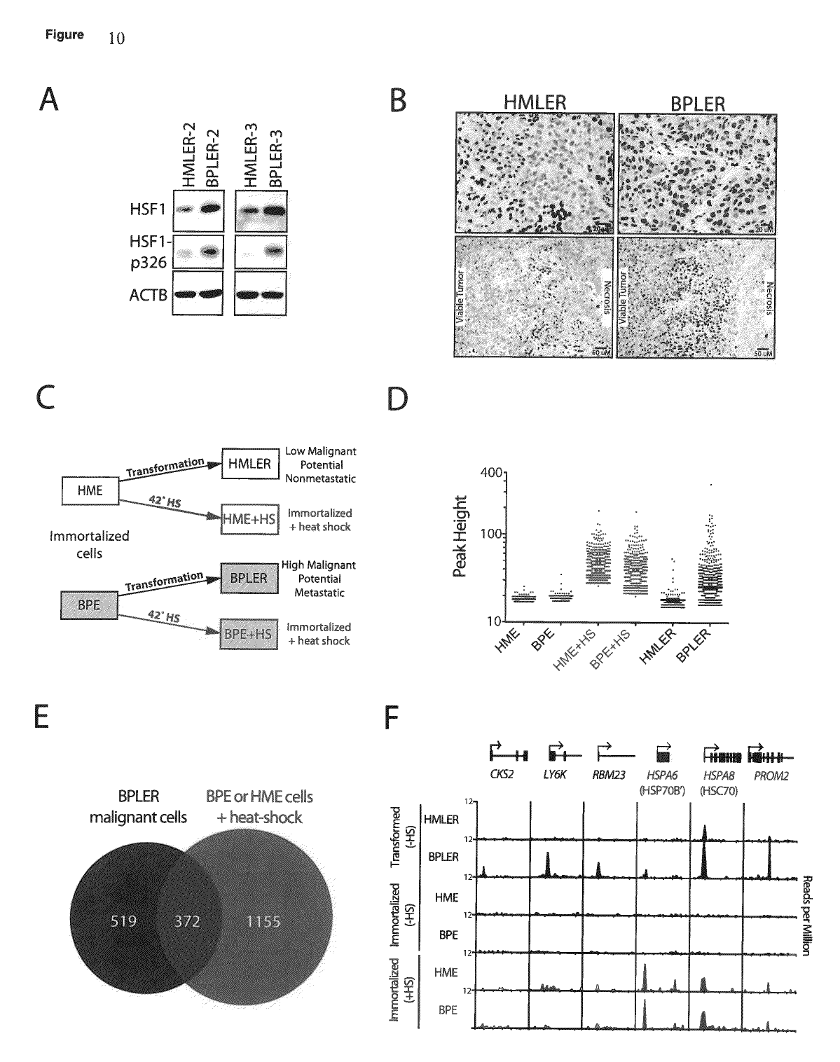

We asked if HSF1 expression differed in the highly malignant BPLER and the much less malignant HMLER breast cancer cells. We used two sets of such cells, each pair derived from a different donor. In both, HSF1 protein expression was higher in the more malignant member of the pair, BPLER cells (FIG. 10A). BPLER cells also had more phosphoserine-326-HSF1, a well established marker of HSF1 activation (Guettouche et al., 2005), than HMLER cells (FIG. 10A).

To determine if these differences in HSF1 were simply an artifact of growth in cell culture, we implanted the cells into immunocompromised mice and allowed them to form tumors. HSF1 immunostaining was weak in the HMLER tumors. Moreover, it was largely restricted to nonmalignant, infiltrating stroma and to tumor areas bordering necrosis (FIG. 10B), indicating that microenvironmental stress can influence the activation of HSF1. In BPLER tumors, however, HSF1 staining was strong, nuclear localized and very uniform (Figures O1B and 17A). Thus, the dramatic difference in HSF1 expression we observe between BPLER and HMLER cells is due to stable, cell-autonomous factors intrinsic to these distinct cell types (Ince et al., 2007).

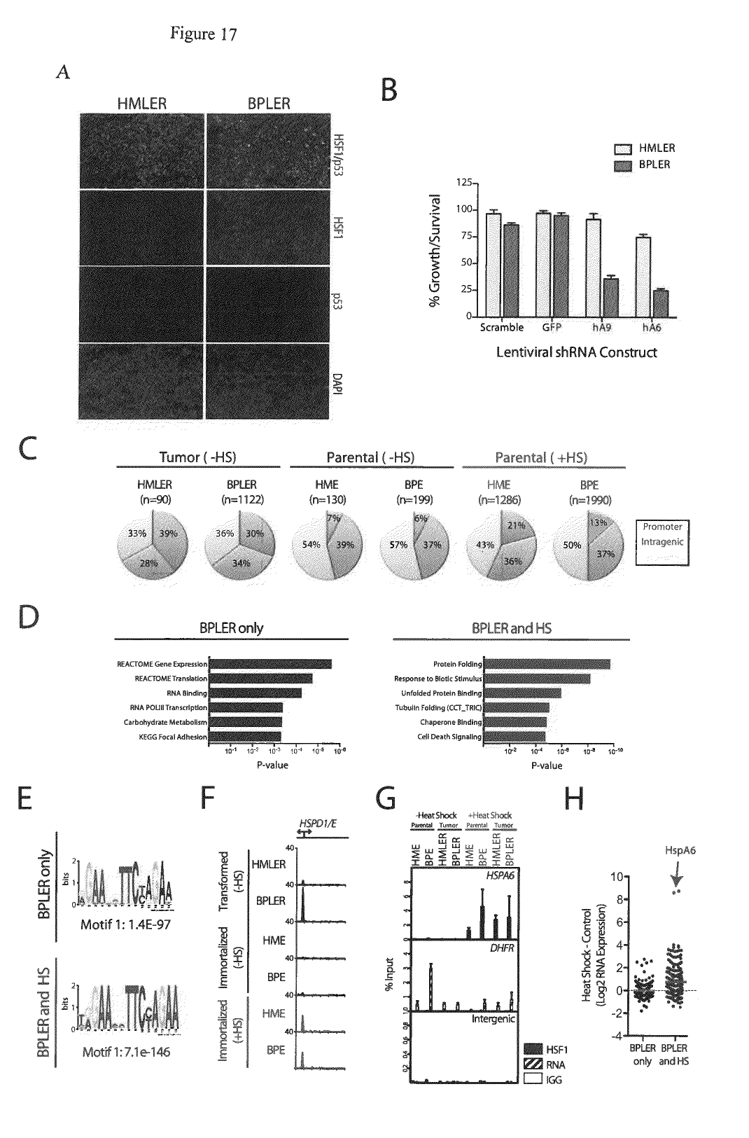

Given this evidence for the activation of HSF1 in BPLER cells, we asked if they were more dependent on HSF1 than HMLER for growth and survival. Neither cell type was affected by negative control shRNA. With two independent shRNA that knockdown HSF1 expression, however, cell growth and viability were far more strongly reduced in BPLER than HMLER cells (FIG. 17B).

### Example 10

### HSF1 Genome Occupancy in Cancer is Distinct from Heat-Shock

To determine if the transcriptional program driven by HSF1 in highly malignant cells differs from that driven by a classical thermal stress, we used chromatin immunoprecipitation coupled with massively parallel DNA sequencing (ChIP-Seq) (Johnson et al., 2007), characterizing HSF1 binding sites genome-wide. We first assessed the immortalized non-transformed progenitor cells, HME and BPE, grown at 37° C. or following a 42° C. heat shock (FIG. 10C). We then related these profiles to the transformed HMLER and BPLER cells grown at 37° C.

In the HME and BPE parental cell lines, a limited number of genes were bound by HSF1 in the absence of heat shock, and these were bound weakly (FIG. 10D; Table T1). Heat shock drove robust binding of HSF1 to ˜800 genes in HME cells and to ˜100 genes in BPE cells (FIG. 10D; Table T1). These observations are consistent with a previous report that a large number of genes are bound by HSF1 in the mammalian heat-shock response (Page et al., 2006).

A small number of genes were bound by HSF1 under basal conditions in the transformed cells with low malignant potential, HMLER (37° C.; FIG. 10D). However, binding was more localized to promoter regions than in the parental cells (FIG. 17C), suggesting some low level of HSF1 activation (MacIsaac et al., 2010). In sharp contrast, in the metastatic and highly tumorigenic BPLER cells, we identified ˜900 genes bound by HSF1 at 37° C. (FIG. 10D; Table T1).

Surprisingly, a full 60% of the genes bound by HSF1 in BPLER cells were not bound in non-transformed parental lines, even after heat-shock (FIG. 10E). Examples included (FIG. 10F): cdk (cyclin-dependent kinase) interacting protein, CKS2, which enables proliferation under conditions of replicative stress common to malignant cells (Liberal et al., 2011); LY6K which encodes a glycosylphosphatidyl-inositol (GPI)-anchored membrane protein implicated as a biomarker in lung and esophageal carcinomas (Ishikawa et al., 2007; Maruyama et al., 2010); and RBM23, which encodes an RNA-binding protein implicated in the regulation of estrogen-mediated transcription (Dowhan et al., 2005). Using the Molecular Signatures Database (MSigDB) (Subramanian et al., 2005) Applicants found that the genes bound uniquely in the BPLER cells were most highly enriched in protein translation, RNA binding, metabolism, cell adhesion (FIG. 17D; Table T2A) and other processes vital in supporting the malignant state (Makrilia et al., 2009; Silvera et al., 2010; Vander Heiden et al., 2009).

We analyzed the 100 bp genomic regions surrounding the peaks of HSF1 binding unique to BPLER cells using the ab initio motif discovery algorithm MEME (Machanick and Bailey, 2011). The canonical heat-shock element (HSE) was highly enriched in the HSF1-bound regions (p-value=1.4×10⁻⁹⁷; FIG. 17E) strongly suggesting the genes that are constitutively bound by HSF1 in malignant cells are bona fide HSF1-binding targets.

The remaining 40% of genes bound by HSF1 in BPLER cells under basal conditions were also bound in the parental lines following heat-shock. As expected, these genes included many classical heat-shock genes, and were enriched for protein folding categories (FIG. 17E; Table T2B). Examples included HSPA8, which encodes the constitutively expressed HSC70 protein, and HSPD1/E1, which encodes HSP60 and HSP10 (FIG. 17F).

Notably, for many of the genes bound in both cancer and heat shock, HSF1 binding differed quantitatively. For example, the strongly heat-shock inducible HSPA6 gene (encoding HSP70B′) was highly bound in parental lines upon heat shock but only weakly bound in BPLER cells at 37° C. (FIGS. 10F, 17G and 17H). Conversely, PROM2, which encodes a basal epithelial cell membrane glycoprotein (Fargeas et al., 2003), was weakly bound by HSF1 in parental lines following heat-shock, but highly bound in BPLER cells (FIG. 1F). Thus, HSF1 engages a regulatory program in the highly malignant state that is distinct from the classic heat-shock response. To further assess the functional significance of the HSF1 cancer program, we asked if the genes comprising this program played a significant role in malignancy, using unbiased data from an independent investigation. The Elledge lab recently conducted a whole genome siRNA screen to identify genes that are required to maintain growth when cells are transformed with a malignantly activated Ras gene (Luo et al., 2009). Among the ˜1600 genes identified in this screen our HSF1-bound gene set was very strongly enriched (73 gene overlap; p Value=7.95 e⁻¹⁵, Table T4G). The HSF1-bound genes we identified as unique to the malignant state were more strongly enriched (Table T4H, 49 gene overlap; p Value=1.1 e⁻¹²) than those shared with heat-shocked cells (Table T4I, 24 gene overlap; p Value=0.0004), but both sets of genes were important in supporting the malignant state.

### Example 1

### HSF1 Regulates Transcription of the Genes it Binds in Malignant Cells

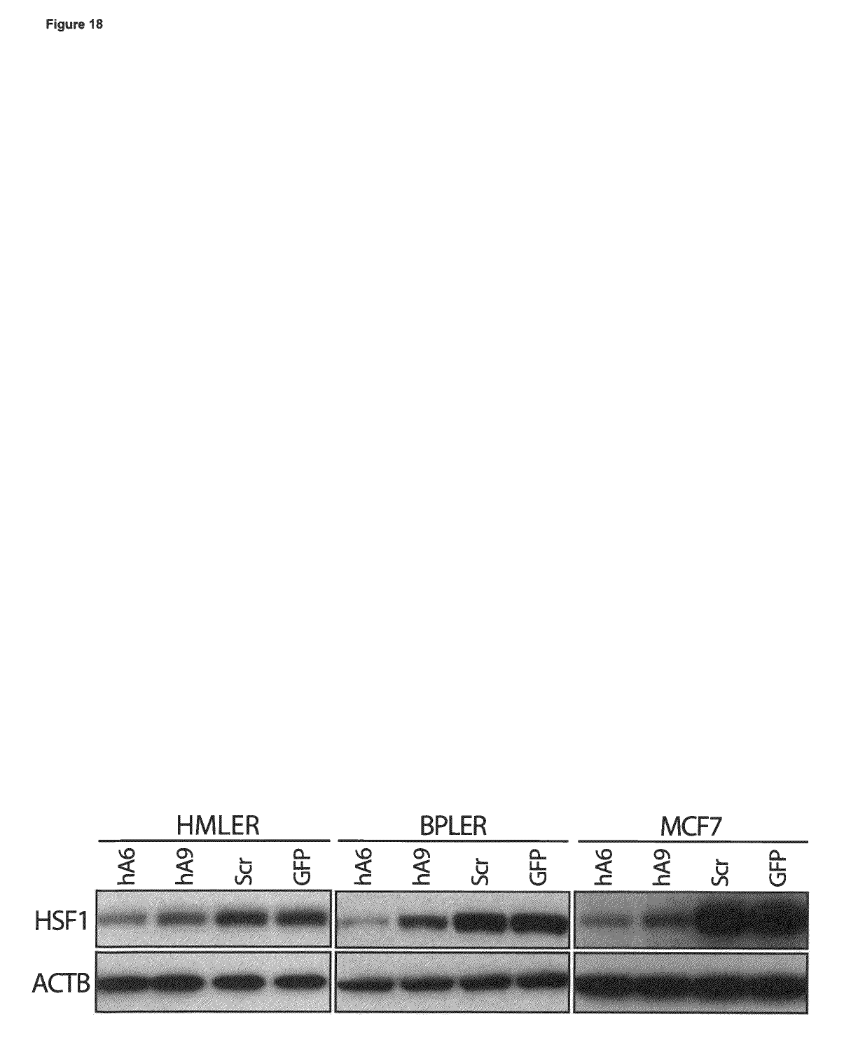

To investigate the consequences of HSF1 occupancy on gene expression, we compared RNA profiles in HMLER and BPLER cells transduced with control shRNA hairpins to those transduced with hairpins that knockdown HSF1. As we previously reported, the growth and survival of malignant cells is compromised by prolonged depletion of HSF1 (Dai et al., 2007). Therefore, we only analyzed mRNA expression in the early stages of shRNA inhibition, where HSF1 knockdown was still incomplete (FIG. 18) but cell viability was unimpaired. These data likely provide a conservative assessment of the effects of HSF1 on gene expression in malignant cells.

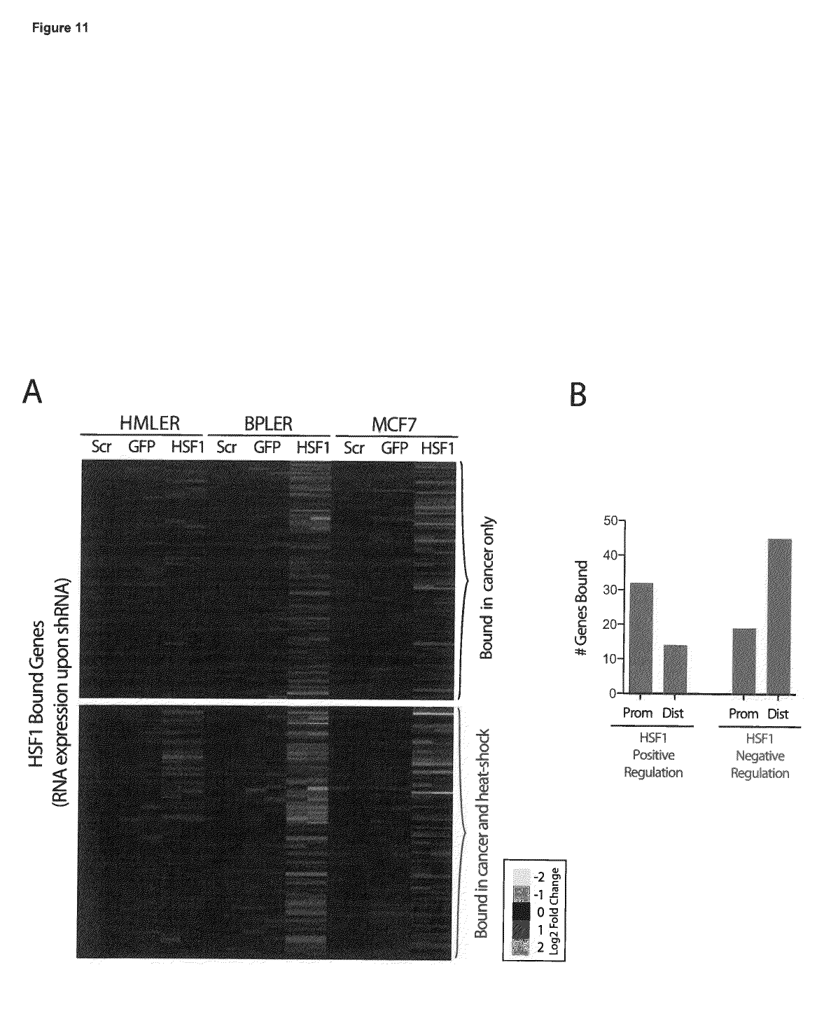

Control hairpins that did not reduce HSF1 levels (Scr and GFP; FIG. 18), had minimal effects on the expression of HSF1-bound genes (FIG. 11A; Table T3). Targeted hairpins that did reduce HSF1 had a minor impact in HMLER cells but markedly changed expression in BPLER cells. The expression of some genes decreased and others increased, indicating that some HSF1-bound genes were positively regulated by the transcription factor while others were negatively regulated. Genes unique to the malignant state and those bound during heat shock were affected equivalently. For example, expression of the malignancy-associated genes CKS2 and RBM23 and the heat-shock protein genes HSPA8 (HSC70) and HSP90AA1 (HSP90) were all reduced (by ˜50%) following HSF1 knockdown (Table T3).

Relating the effects of the hairpins on gene expression to our earlier ChIP-Seq analysis, ˜70% of genes positively regulated by HSF1 were bound at the promoter while only ˜30% of these genes were bound in distal regions (FIG. 11B). Genes that were negatively regulated by HSF1, showed the opposite pattern (FIG. 11B). This observation (p-value=0.00004) suggests that the direction of regulation (positive versus negative) in these cells is clearly influenced by the location of the HSF1-binding site.

We also examined the effects of HSF1 knockdown on gene expression in MCF7 cells. In contrast to genetically engineered HMLER and BPLER cells, the MCF7 line was established from a human breast cancer metastasis (Soule et al., 1973). Moreover, as an estrogen receptor positive (ER+) line, its biology is fundamentally distinct from the hormone-receptor negative HMLER and BPLER cell lines. Despite these differences, the pattern of changes in gene expression caused by HSF1 knockdown was very similar in BPLER cells and MCF7 cells for HSF1 targets (FIG. 11A).

### Example 12

### HSF1 Gene Occupancy is Conserved Across a Broad Range of Common Human Cancer Cell Lines

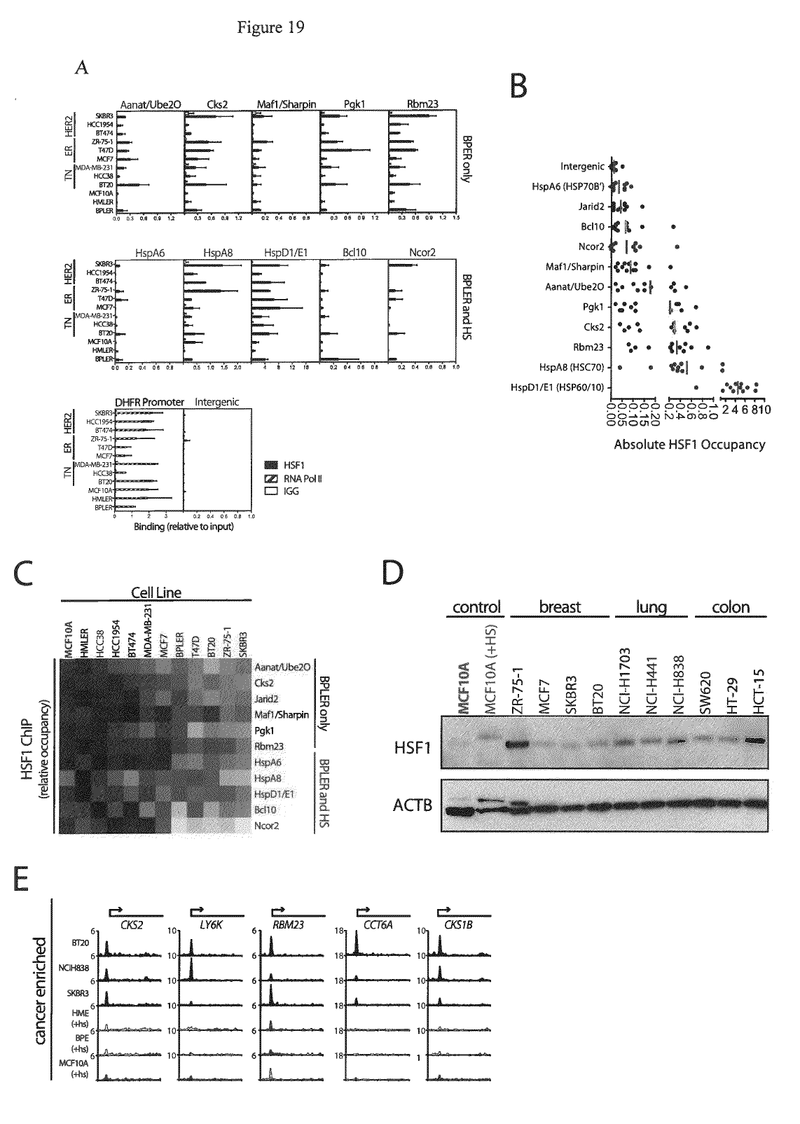

Next we used ChIP-qPCR to monitor HSF1 binding to a representative set of the HSF1-target genes in cell lines derived from patients with breast cancer. We used nine well-studied cancer lines (including MCF7 cells) representing all three major categories of breast cancer: ER+, HER2+ and Triple Negative (TN). Under basal conditions (at 37° C.) we detected HSF1 binding in each of the major breast cancer subtypes (FIG. 19A). A range of binding intensities was observed. Most notably, however, the distinct pattern of HSF1 gene occupancy in the highly malignant engineered BPLER cells was also present in these naturally-arising malignant cells. In such cell lines, HSF1 bound to genes (such as CKS2 and RBM23) that we had previously identified as bound well in BPLER cells but not in the non-transformed parental lines. Similar to our results in the BPLER/HMLER cells system, HSPD1/E1 was highly bound by HSF1 in all cell lines, but the strongly heat-shock inducible HSPA6 gene was minimally bound in the cancer lines under basal conditions (37° C.; FIGS. 19A, 19B and 19C). We also analyzed HSF1 binding in the non-tumorigenic breast cell line MCF10A. Comparable to the low malignancy HMLER cells, MCF10A cells had low levels of HSF1 occupancy across all genes examined (FIGS. 19A and 19C).

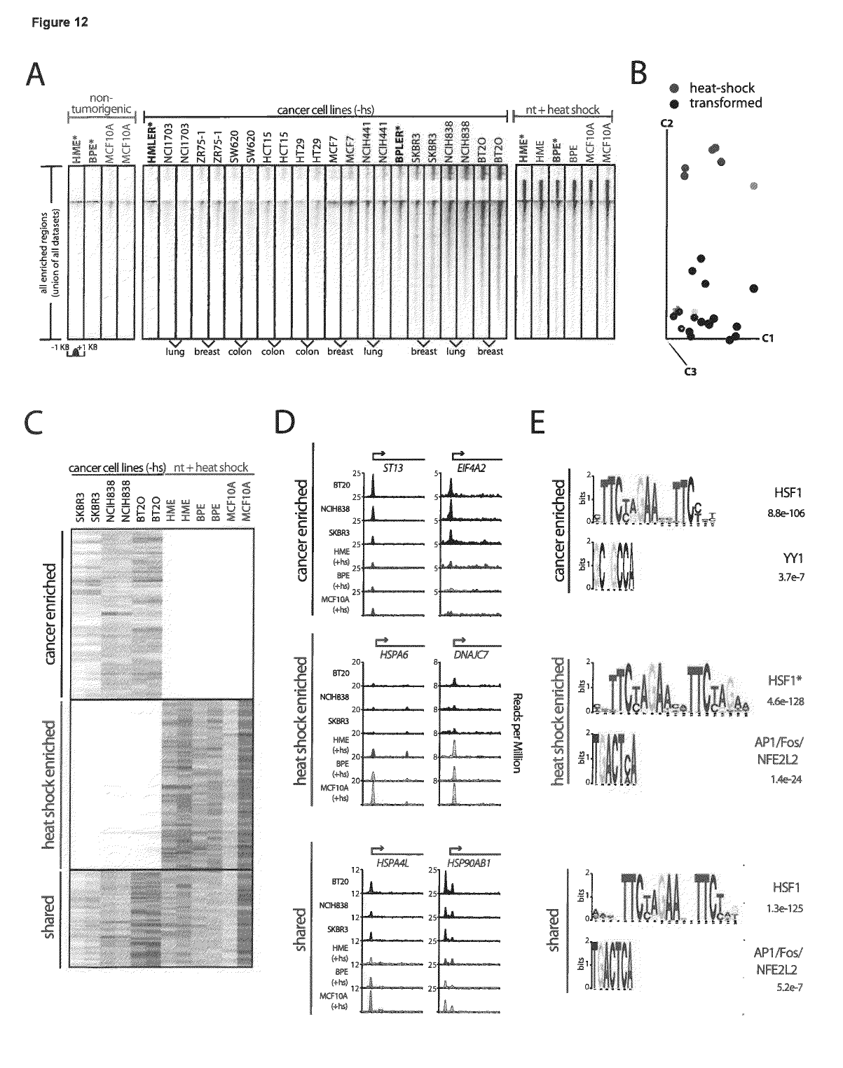

These ChIP-PCR data spurred us to employ ChIP-Seq to generate high-resolution maps of HSF1 occupancy, and to do so in a panel of human tumor lines that extended to other types of malignancy (FIGS. 12A and 19D). We assessed HSF1 binding in duplicate samples of four breast, three lung and three colon cancer cell lines, thus covering the human cancers with the highest total mortality in the developed world. We compared these cancer cells grown at 37° C. with our data from the non-tumorigenic cell lines HME and BPE and weakly tumorigenic HMLER cells. As an additional point of comparison we performed ChIP-Seq analysis on the non-tumorigenic MCF10A cell line grown either at 37° C. or following a 42° C. heat-shock.

After heat shock, MCF10A cells exhibited an HSF1-binding profile that was comparable to that of heat-shocked HME and BPE cells. In the absence of heat shock the overall magnitude of HSF1 binding in all of the non-tumorigenic cell lines (nt) was uniformly very weak and the total number of bound genes was small (FIG. 12A; Table T1). In contrast, in the cancer lines a range of HSF1 binding was observed at 37° C. (FIG. 12A). For example, robust binding was observed in the lung adenocarcinoma line NCI-H838 and in the TN breast carcinoma line BT20. Less pronounced overall binding was seen in others lines such as the weakly malignant HMLER. Binding in BPLER cells was intermediate.

Irrespective of the level of binding, the distribution of HSF1 occupancy on a genome-wide scale was remarkably similar among the cancer cell lines and distinct from the pattern of binding in the heat-shocked cells (FIG. 12A). The global nature of the differences in the HSF1-binding profiles between the heat-shocked and malignant state was confirmed using principal component analysis (PCA; FIG. 12B). This unsupervised method of clustering sets of data clearly distinguished one cluster containing all cell lines exposed to heat-shock and a second cluster containing all cancer cell lines.

Data from these multiple cell lines allowed us to confidently identify regions of HSF1 binding that were strong in cancer cells but not in heat-shocked cells, weak in cancer but strong in heat-shock or similarly strong in both (FIG. 12C). Examples of genes that were strongly bound in cancer but not in heat shock included CKS2, LY6K, RBM23, CCT6A, CKS1B, ST13, EIF4A2 (FIGS. 19E and 12D). Genes that were weakly bound in cancer lines but strongly bound in heat shock included HSPA6 and DNAJC7 (FIG. 12D). Genes that were strongly bound in both cell types included HSPA4L and HSP90AB1 (FIG. 12D).

We performed motif analysis to evaluate the 100 bp genomic regions surrounding the peaks of HSF1 binding in each of these groups. The HSE, comprised of adjacent inverted repeats of 5′-nGAAn-3′, was the most enriched motif in all three groups (FIG. 12E). The regions strongly bound in cancer but not heat-shock were enriched in HSEs that had three such repeats (p-value=8.8×10⁻¹⁰⁶). They were also enriched in binding elements for YY1, the so called “ying-yang” transcription factor which is involved in activating and repressing a broad range of genes (p-value=3.7×10⁻⁷). The regions strongly bound in heat-shocked cells but not cancer were enriched for expanded HSEs, with a fourth 5′-nGAAn-3′ repeat (p-value=4.6×10⁻¹²⁸). They also were enriched in an AP1/Fos/NRF2 (NFE2L2) binding site (p-value=1.4×10⁻²⁴) as previously reported for mammalian heat-shock genes. This variation in binding motifs suggests the involvement of distinct co-regulators in establishing differential patterns of HSF1 occupancy. The regions strongly bound by HSF1 in both cancer and in heat shock had features of both groups. They were enriched for HSEs with three inverted repeats (p-value=1.3×10⁻¹²⁵). They were not enriched for the YY1 sites but were enriched for the AP1/Fos and NRF2 binding site (p-value=5.2×10⁷).

### Example 13

### HSF1-Bound Genes Form Distinct, Coordinately-Regulated Modules

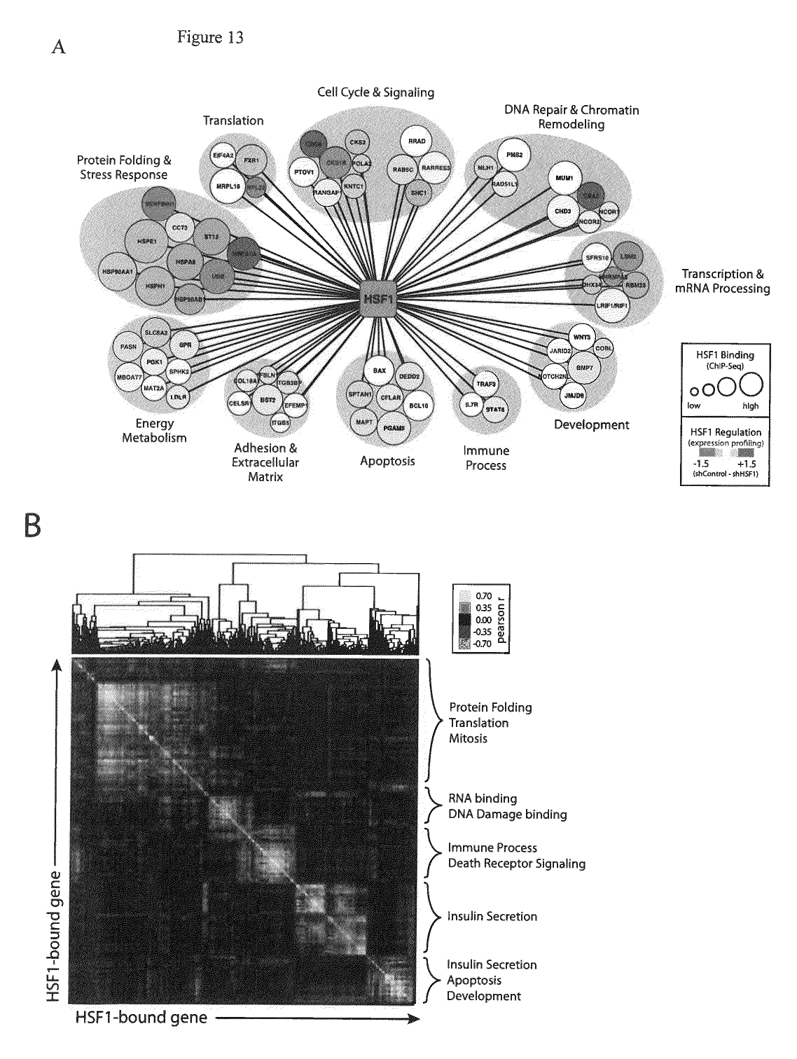

Integrating our diverse data sets (FIG. 13A), revealed a direct and pervasive role for HSF1 in cancer biology. Extending far beyond protein folding and stress, HSF1-bound genes were involved in many facets of tumorigenesis, including the cell cycle, apoptosis, energy metabolism and other processes. To gain a more global view of the relationship between the genes most strongly bound by HSF1 in cancer cell lines, we generated an RNA expression correlation matrix through meta-analysis of pre-existing data sets (FIG. 13B). We used the UCLA Gene Expression Tool (UGET) (Day et al., 2009) to query the extent to which the expression of each HSF1-bound gene correlated with every other HSF1-bound gene across the ˜12,000 human expression profiles generated with Affymetrix HG U133 Plus 2.0 arrays and available through the Celsius database (Day et al., 2007). Hierarchical clustering of this gene-gene correlation matrix revealed five major transcription modules (FIG. 13B).

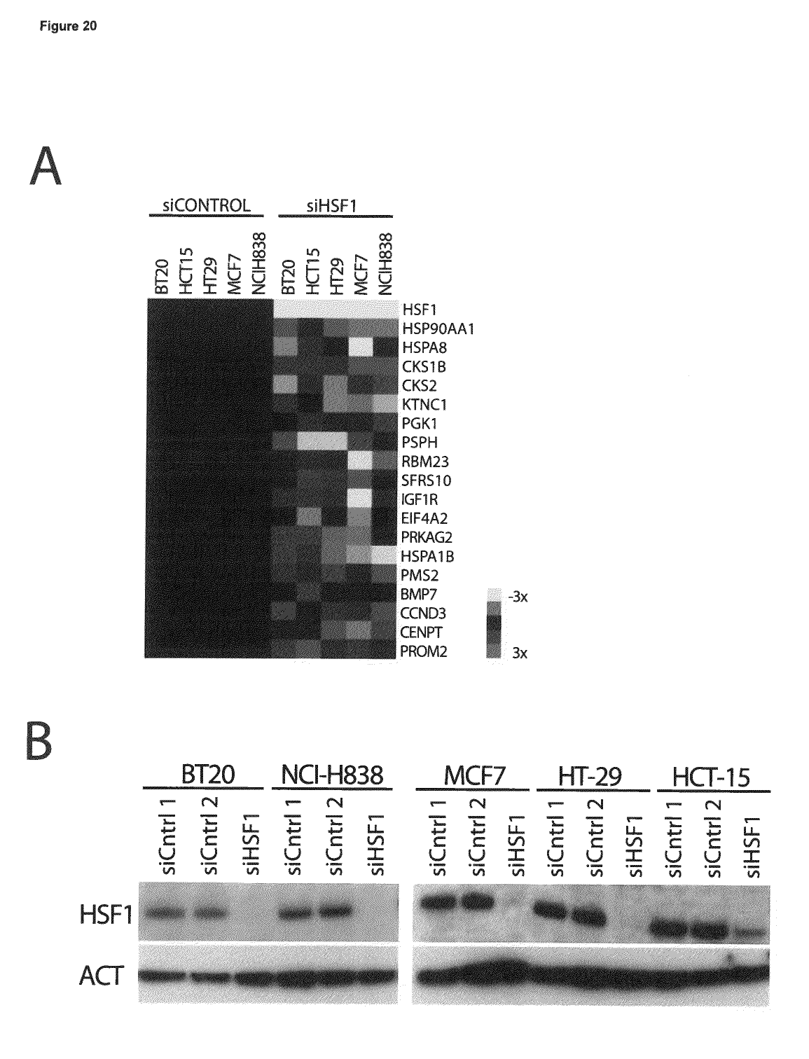

The largest module was enriched for protein folding, translation and mitosis. Genes within this dominant module showed the strongest positive correlation with the expression of HSF mRNA itself. Many of these genes had indeed proven to be regulated by HSF1 in our HSF1 shRNA knockdown experiments (FIGS. 1, 13A and 20). A second, smaller module was positively correlated with the first and strongly enriched for RNA binding genes. Many of these genes, too, were positively regulated by HSF1 in our knockdown experiments (FIGS. 11 and 13A and 20). The remaining three modules (center to lower right of the matrix) were enriched for processes involved in immune functions, insulin secretion and apoptosis. All three of these modules were negatively correlated with the largest module, suggesting negative regulation by HSF1.

### Example 14

### Activation of HSF1 in a Broad Range of Cancer Specimens Taken Directly from Patients

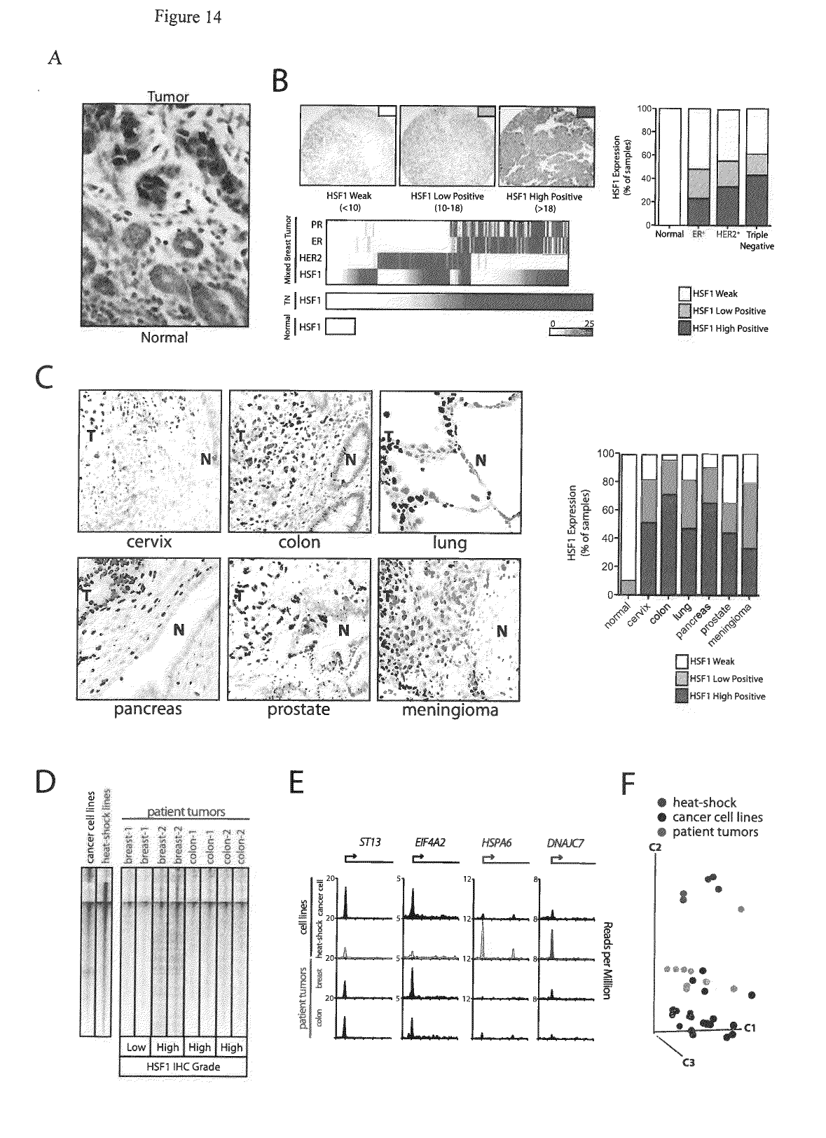

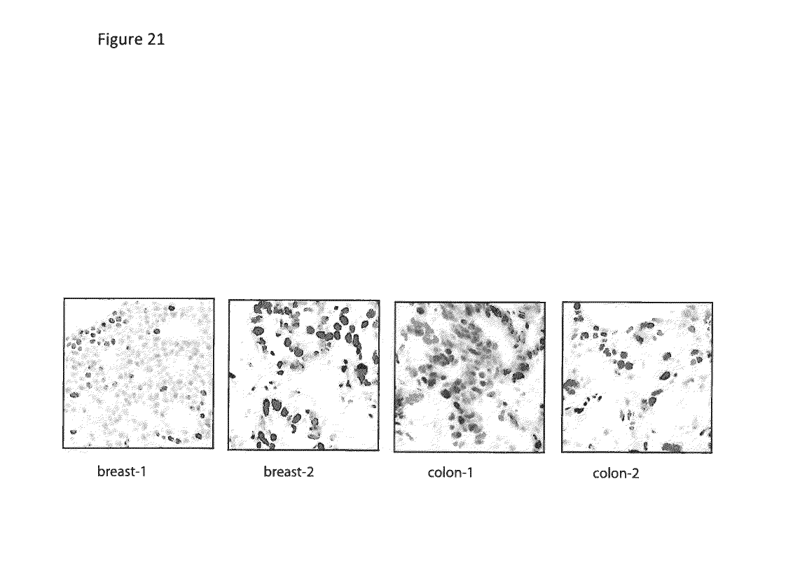

As described above, we evaluated HSF1 expression and localization in a cohort of breast cancer patients culled from the Nurses' Health Study (NHS) (Santagata et al., 2011). In that work, HSF1 was cytoplasmic and expressed at low levels in normal breast epithelial cells but it accumulated in the nucleus of the majority of tumor specimens. Here, we have confirmed that finding (FIGS. 14A, 14B and 21), combining samples from two independent breast cancer collections representing all three major clinical subtypes (see Methods).

Next, because our ChIP-Seq analysis showed that the HSF1 cancer program is engaged not just in breast cancer lines but also in colon and lung cancer cell lines, we examined more than 300 formalin-fixed surgical specimens taken directly from patients. We included not only colon and lung cancer but also a wide variety of other tumor types. Normal cells adjacent to the tumor demonstrated low HSF1 levels and cytoplasmic localization of the protein. In contrast, high-level nuclear expression of HSF1 was common across every cancer type we examined, including carcinomas of the cervix, colon, lung, pancreas and prostate as well as mesenchymal tumors such as meningioma (FIG. 14C). In these tumors, expression was generally uniform across the sample, with nearly all tumor cells expressing similar levels of nuclear HSF1.

To further confirm that the high-level nuclear localization of HSF1 detected by immunostaining was truly indicative of its activation, we obtained human tumor samples from breast and colon adenocarcinomas that had been cryopreserved and were of a quality suitable for ChIP-Seq analysis (FIGS. 14D and 21). Despite the potential confounding factors such as cell-type heterogeneity due to the presence of blood and stromal elements, areas of necrosis and micro-environmental stress, etc., the distinct HSF1-binding profile we established with cancer cell lines was conserved. Genes that were strongly bound by HSF1 in cancer lines but weakly bound after heat shock (such as ST13 and EIF4A2), were also strongly bound in tumor samples (FIG. 14E). Genes that were weakly bound by HSF1 in cancer lines but strongly bound after heat shock (such as HSPA6 and DNAJC7) were also weakly bound in tumor samples (FIG. 14E). These global similarities in HSF1-binding profiles between cancer cell lines and tumor samples, as well as their divergence from heat shock profiles, were confirmed by principal component analysis (FIG. 14F).

### Example 15

### An HSF1-Cancer Signature Identifies Breast Cancer Patients with Poor Outcome

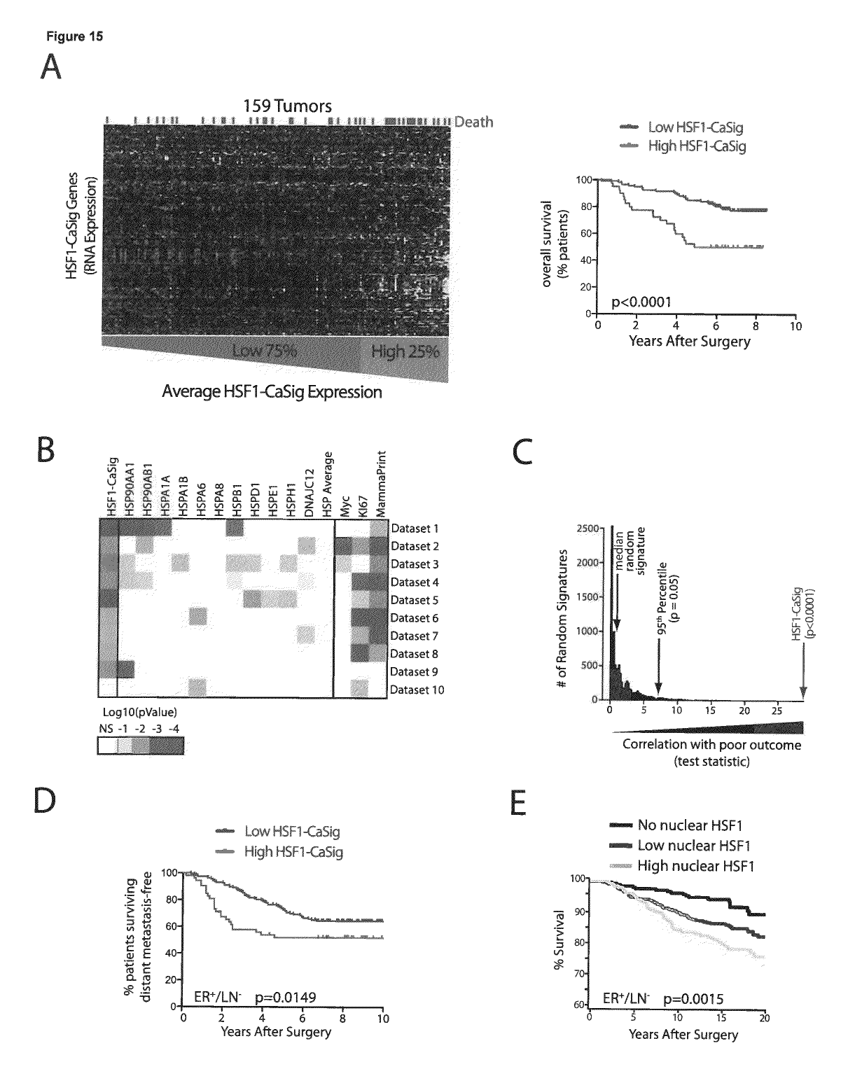

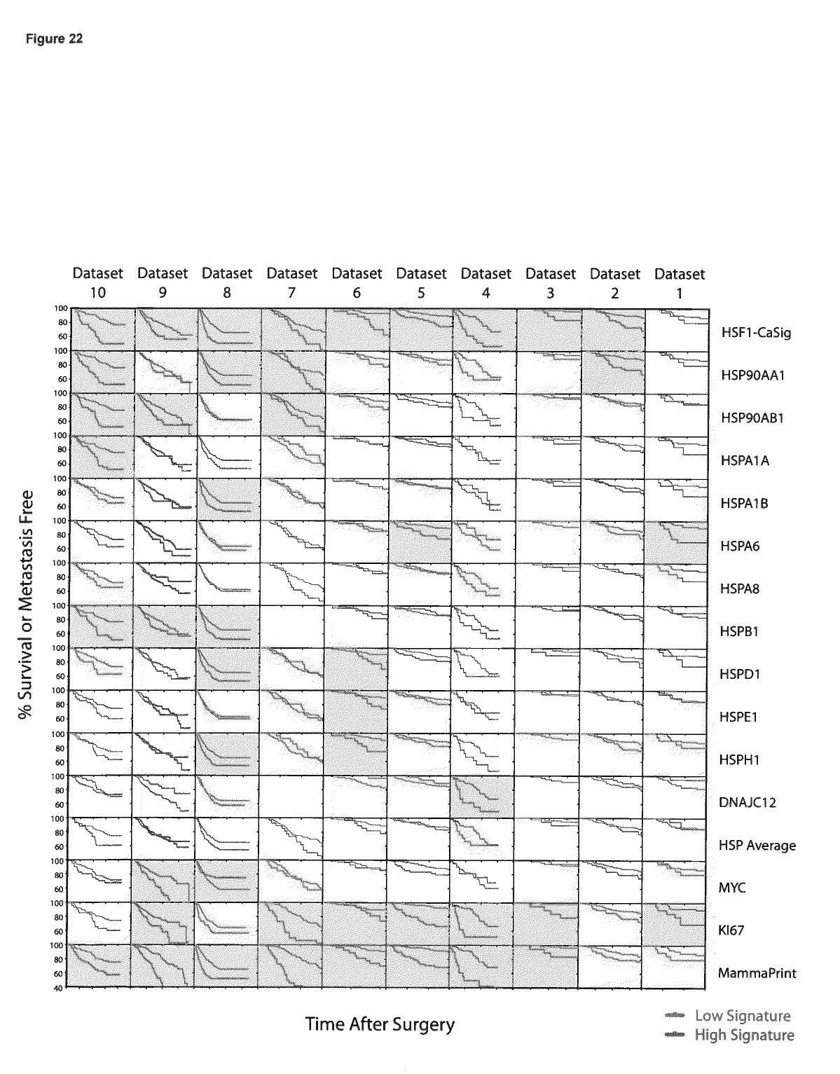

In our analysis of the Nurses' Health cohort, HSF1 overexpression and nuclear localization was associated with reduced survival (see Examples 2-7 above; see also Santagata et al, 2011a). To acquire more precise and molecularly defined information about the effects of HSF1 activation in cancer, we asked if malignant potential and long-term outcomes correlate with the HSF1 transcriptional program identified above. We distilled an “HSF1-cancer signature” of 456 genes that were bound by HSF1 near their transcription start sites (FIG. 11). Expression of these genes (Table T4C) was interrogated in ten publicly available mRNA datasets derived from breast cancer patients that had been followed for an average of 7.58 years and had known clinical outcomes (referenced in Table T5). In total, these cohorts encompassed nearly 1,600 individuals of diverse national and ethnic origin. We divided each dataset into two groups, those with high (top 25%) and those with low (bottom 75%) expression of the HSF1-cancer signature. We performed Kaplan-Meier analysis independently on each dataset to assess potential associations between the HSF1I-cancer signature and patient outcome: metastasis-free, relapse-free, or overall survival, depending on the reported outcome parameter for that dataset. One representative analysis is presented in FIG. 15A, the remainder are shown in FIG. 22.

High expression of our HSF1-cancer signature had a remarkable correlation with poor prognosis (HSF1-CaSig; FIGS. 15B and 22). In 9 of 10 independent datasets reported over the past 10 years, the P values ranged from 0.05 to <0.0001. The one dataset that did not demonstrate a significant correlation contained, by far, the highest percentage of ER-negative tumors (Table T5), a typically aggressive subtype of breast cancer. In these generally poor prognosis tumors, HSF1 was more highly and uniformly activated (FIG. 14B). Thus, it is not that HSF1 activation is unimportant in these tumors, but rather that the HSF1-cancer signature per se loses prognostic power. To investigate further, we stratified the two datasets (van de Vijver et al., 2002; Wang et al., 2005) with the largest number of patients by ER status. Indeed, our HSF1-cancer signature was more uniformly increased in the ER-negative population.

Next, we considered a recent finding that many published cancer signatures are not significantly better outcome predictors than random signatures of identical size (Venet et al., 2011). We performed Kaplan-Meier analysis on independent datasets to evaluate associations between 10,000 individual randomly generated gene signatures and patient outcome (example shown in FIG. 15C). A meta-analysis of the breast datasets showed that the HSF1-CaSig outperformed all 10,000 random gene signatures (Monte Carlo p Value across breast datasets <0.0001, Table T8.) A meta-analysis of the lung and colon datasets showed that the HSF1-CaSig outperformed all 10,000 random gene signatures (Monte Carlo p Value across lung and colon datasets <0.0001, Table T8. Table T8 shows a Monte Carlo p-value of the HSF1-CaSig for each dataset and also contains log-rank p-value and test statistic of the HSF1-CaSig, the median and 95th percentile (corresponding to a p-value of 0.05) log-rank p-value and test statistic of the random signatures.

Our HSF1-cancer signature was more significantly associated with outcome than other well established prognostic indicators (FIGS. 15B and 22) including the oncogene MYC, the proliferation marker Ki67 and even MammaPrint, an expression-based diagnostic tool used in routine clinical practice (Kim and Paik, 2010). Because various HSPs have been implicated as prognostic markers for a range of cancers including breast cancer (Ciocca and Calderwood, 2005), we also tested many individual HSP transcripts for possible association with outcome. None of these genes, or even a panel of HSP genes, was as strongly associated with poor outcome as our broader HSF1-cancer signature (FIGS. 15B and 22).

### Example 16

### HSF1 Activation is an Indicator of Poor Outcome in Early Breast Cancer

At the time of diagnosis, the majority of breast cancer patients have ER+ tumors and early-stage disease (ER⁺/lymph-node negative tumors). A small fraction of these patients will experience a recurrence and might benefit from more aggressive treatment, but it is currently very difficult to identify them in advance. We found that our HSF1-cancer signature was significantly associated with metastatic recurrence in women initially diagnosed with ER⁺/lymph node negative tumors (p-value=0.0149) (FIG. 15D).

To confirm the prognostic value of HSF1 in this particularly challenging population, we returned to the Nurses' Health Study cohort, because it provides one of the largest collections of patients with ER⁺/lymph node negative tumors for evaluation (n=947), and has the longest patient follow up. Because RNA samples are not available from this collection (initiated in 1976) we could assess only the levels and nuclear localization of HSF1. Survival decreased as HSF1 nuclear levels increased in a dose-dependent manner (p-value=0.0015; FIG. 15E). This finding was validated by multivariate analysis which showed high level nuclear HSF1 to be associated with a nearly 100% increase in mortality (Table T6).

### Example 17

### HSF1-Cancer Signature is Associated with Poor Outcome in Diverse Human Cancers

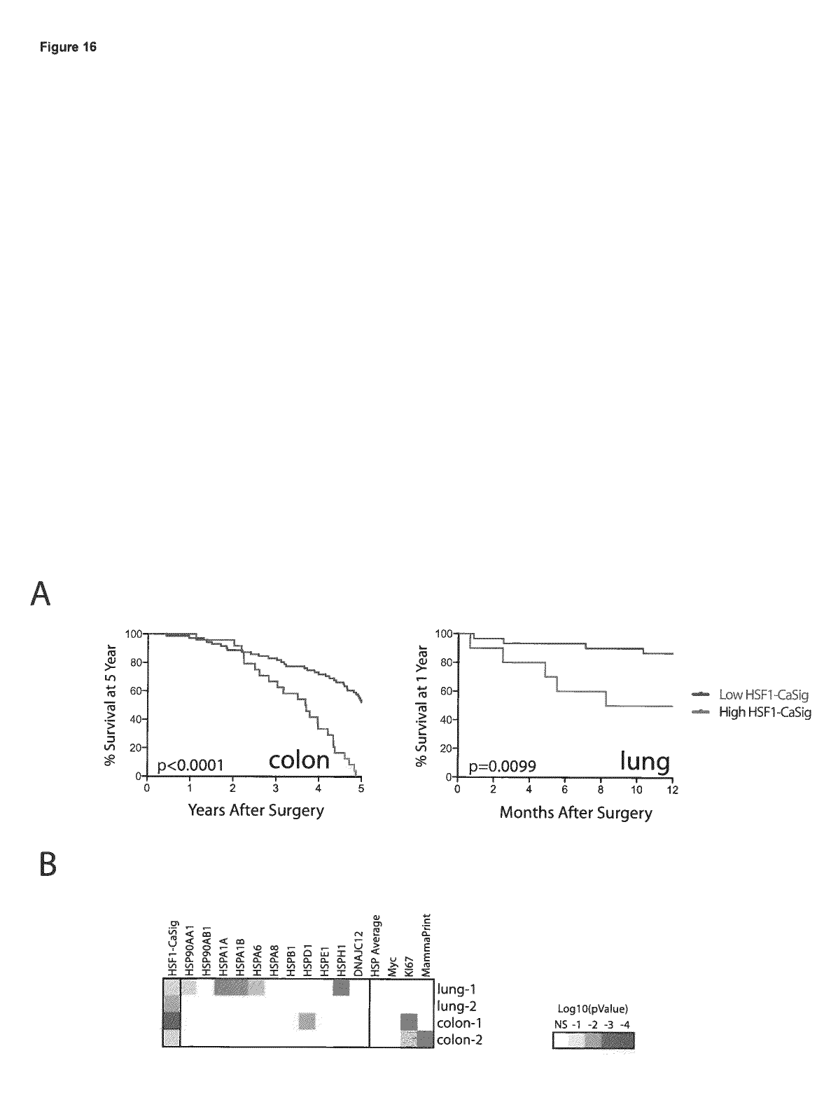

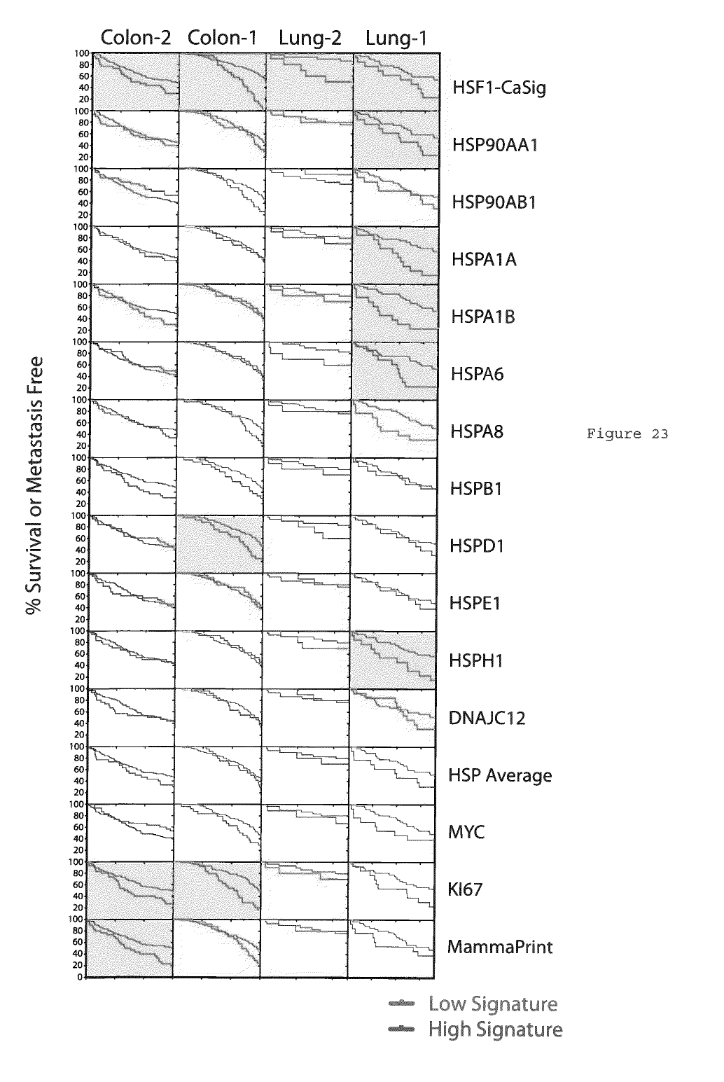

Finally, we asked if the HSF1-cancer signature might have prognostic value beyond breast cancer. Analyzing multiple independent gene expression datasets that include outcomes data, increased expression of the HSF1 cancer program in colon and lung cancers was strongly associated with reduced survival (FIGS. 16A and 16B). The HSF1-CaSig outperformed all 10,000 random gene signatures in these datasets (Monte Carlo p Value across datasets <0.0001. Again, our HSF1-cancer signature was more significantly associated with outcome than any individual HSP transcript or even a panel of HSP genes (FIGS. 16B and 23). As expected, the MammaPrint expression signature, which was computationally derived using breast cancers, was a poor indicator of outcome in lung and colon cancers (significant in 1 of 4 datasets). Additional HSF1 signatures containing positively regulated genes (from Module 1 and 2 of our gene-gene correlation analysis; HSF1-CaSig2) or containing both positively and negatively regulated genes (HSF1-CaSig3) were also strongly associated with patient outcome across tumor types. Table T9 contains log-rank p-values for each of the three HSF1-CaSig classifiers for each of the 14 datasets (10 breast, 2 lung, 2 colon). We conclude that the HSF1 cancer program that we have identified supports the malignant state in a diverse spectrum of cancers because it regulates core processes rooted in fundamental tumor biology that ultimately affect outcome.

### REFERENCE LIST 2

Anckar, J., and Sistonen, L. (2011). Regulation of HSF1 Function in the Heat Stress Response: Implications in Aging and Disease. Annu Rev Biochem 80, 1089-1115.
Boellmann, F., and Thomas, R. S. (2010). The identification of protein kinase C iota as a regulator of the mammalian heat shock response using functional genomic screens. PLoS One 5, e11850.
Chiang, W. C., Ching, T. T., Lee, H. C., Mousigian, C., and Hsu, A. L. (2012). HSF-1 regulators DDL-1/2 link insulin-like signaling to heat-shock responses and modulation of longevity. Cell 148, 322-334.
Christians, E. S., Yan, L. J., and Benjamin, I. J. (2002). Heat shock factor 1 and heat shock proteins: critical partners in protection against acute cell injury. Crit Care Med 30, S43-50.
Ciocca, D. R., and Calderwood, S. K. (2005). Heat shock proteins in cancer: diagnostic, prognostic, predictive, and treatment implications. Cell Stress Chaperones 10, 86-103.
Dai, C., Whitesell, L., Rogers, A. B., and Lindquist, S. (2007). Heat shock factor 1 is a powerful multifaceted modifier of carcinogenesis. Cell 130, 1005-1018.
Day, A., Carlson, M. R., Dong, J., O'Connor, B. D., and Nelson, S. F. (2007). Celsius: a community resource for Affymetrix microarray data. Genome Biol 8, RI 12.
Day, A., Dong, J., Funari, V. A., Harry, B., Strom, S. P., Cohn, D. H., and Nelson, S. F. (2009). Disease gene characterization through large-scale co-expression analysis. PLoS One 4, e8491.
Dowhan, D. H., Hong, E. P., Auboeuf, D., Dennis, A. P., Wilson, M. M., Berget, S. M., and O'Malley, B. W. (2005). Steroid hormone receptor coactivation and alternative RNA splicing by U2AF65-related proteins CAPERalpha and CAPERbeta. Mol Cell 17, 429-439.
Fargeas, C. A., Florek, M., Huttner, W. B., and Corbeil, D. (2003). Characterization of prominin-2, a new member of the prominin family of pentaspan membrane glycoproteins. J Biol Chem 278, 8586-8596.
Gerlinger, M., Rowan, A. J., Horswell, S., Larkin, J., Endesfelder, D., Gronroos, E., Martinez, P., Matthews, N., Stewart, A., Tarpey, P., et al. (2012). Intratumor heterogeneity and branched evolution revealed by multiregion sequencing. N Engl J Med 366, 883-892.
Guettouche, T., Boellmann, F., Lane, W. S., and Voellmy, R. (2005). Analysis of phosphorylation of human heat shock factor 1 in cells experiencing a stress. BMC Biochem 6, 4.
Hahn, J. S., Hu, Z., Thiele, D. J., and Iyer, V. R. (2004). Genome-wide analysis of the biology of stress responses through heat shock transcription factor. Mol Cell Biol 24, 5249-5256.
Hahn, J. S., and Thiele, D. J. (2004). Activation of the Saccharomyces cerevisiae heat shock transcription factor under glucose starvation conditions by Snf1 protein kinase. J Biol Chem 279, 5169-5176.
Hu, R., Dawood, S., Holmes, M. D., Collins, L. C., Schnitt, S. J., Cole, K., Marotti, J. D., Hankinson, S. E., Colditz, G. A., and Tamimi, R. M. (2011). Androgen receptor expression and breast cancer survival in postmenopausal women. Clin Cancer Res 17, 1867-1874.
Ince, T. A., Richardson, A. L., Bell, G. W., Saitoh, M., Godar, S., Karnoub, A. E., Iglehart, J. D., and Weinberg, R. A. (2007). Transformation of different human breast epithelial cell types leads to distinct tumor phenotypes. Cancer Cell 12, 160-170.
Ishikawa, N., Takano, A., Yasui, W., Inai, K., Nishimura, H., Ito, H., Miyagi, Y., Nakayama, H., Fujita, M., Hosokawa, M., et al. (2007). Cancer-testis antigen lymphocyte antigen 6 complex locus K is a serologic biomarker and a therapeutic target for lung and esophageal carcinomas. Cancer Res 67, 11601-11611.
Jin, X., Moskophidis, D., and Mivechi, N. F. (2011). Heat Shock Transcription Factor 1 Is a Key Determinant of HCC Development by Regulating Hepatic Steatosis and Metabolic Syndrome. Cell Metab 14, 91-103.
Johnson, D. S., Mortazavi, A., Myers, R. M., and Wold, B. (2007). Genome-wide mapping of in vivo protein-DNA interactions. Science 316, 1497-1502.
Kalager, M., Adami, H. O., Bretthauer, M., and Tamimi, R. M. (2012). Overdiagnosis of Invasive Breast Cancer Due to Mammography Screening: Results From the Norwegian Screening Program. Ann Intern Med 156, 491-499.
Khaleque, M. A., Bharti, A., Gong, J., Gray, P. J., Sachdev, V., Ciocca, D. R., Stati, A., Fanelli, M., and Calderwood, S. K. (2008). Heat shock factor 1 represses estrogen-dependent transcription through association with MTA1. Oncogene 27, 1886-1893.
Kim, C., and Paik, S. (2010). Gene-expression-based prognostic assays for breast cancer. Nat Rev Clin Oncol 7, 340-347.
Lee, T. I., Johnstone, S. E., and Young, R. A. (2006). Chromatin immunoprecipitation and microarray-based analysis of protein location. Nat Protoc 1, 729-748.
Lee, Y. J., Lee, H. J., Lee, J. S., Jeoung, D., Kang, C. M., Bae, S., Lee, S. J., Kwon, S. H., Kang, D., and Lee, Y. S. (2008). A novel function for HSF1-induced mitotic exit failure and genomic instability through direct interaction between HSF1 and Cdc20. Oncogene 27, 2999-3009.
Liberal, V., Martinsson-Ahlzen, H. S., Liberal, J., Spruck, C. H., Widschwendter, M., McGowan, C. H., and Reed, S. I. (2011). Breast Cancer Special Feature: Cyclin-dependent kinase subunit (Cks) 1 or Cks2 overexpression overrides the DNA damage response barrier triggered by activated oncoproteins. Proc Natl Acad Sci USA.
Luo, J., Emanuele, M. J., Li, D., Creighton, C. J., Schlabach, M. R., Westbrook, T. F., Wong, K. K., and Elledge, S. J. (2009). A genome-wide RNAi screen identifies multiple synthetic lethal interactions with the Ras oncogene. Cell 137, 835-848.
Machanick, P., and Bailey, T. L. (2011). MEME-ChIP: motif analysis of large DNA datasets. Bioinformatics 27, 1696-1697.
Maclsaac, K. D., Lo, K. A., Gordon, W., Motola, S., Mazor, T., and Fraenkel, E. (2010). A quantitative model of transcriptional regulation reveals the influence of binding location on expression. PLoS Comput Biol 6, e1000773.
Makrilia, N., Kollias, A., Manolopoulos, L., and Syrigos, K. (2009). Cell adhesion molecules: role and clinical significance in cancer. Cancer Invest 27, 1023-1037.
Maruyama, M., Yoshitake, H., Tsukamoto, H., Takamori, K., and Araki, Y. (2010). Molecular expression of Ly6k, a putative glycosylphosphatidyl-inositol-anchored membrane protein on the mouse testicular germ cells. Biochem Biophys Res Commun 402, 75-81.
Meng, L., Gabai, V. L., and Sherman, M. Y. (2010). Heat-shock transcription factor HSF1 has a critical role in human epidermal growth factor receptor-2-induced cellular transformation and tumorigenesis. Oncogene 29, 5204-5213.
Min, J. N., Huang, L., Zimonjic, D. B., Moskophidis, D., and Mivechi, N. F. (2007). Selective suppression of lymphomas by functional loss of Hsf1 in a p53-deficient mouse model for spontaneous tumors. Oncogene 26, 5086-5097.
Morimoto, R. I. (2008). Proteotoxic stress and inducible chaperone networks in neurodegenerative disease and aging. Genes Dev 22, 1427-1438.
Page, T. J., Sikder, D., Yang, L., Pluta, L., Wolfinger, R. D., Kodadek, T., and Thomas, R. S. (2006). Genome-wide analysis of human HSF1 signaling reveals a transcriptional program linked to cellular adaptation and survival. Mol Biosyst 2, 627-639.
Pawitan, Y., Bjohle, J., Amler, L., Borg, A. L., Egyhazi, S., Hall, P., Han, X., Holmberg, L., Huang, F., Klaar, S., et al. (2005). Gene expression profiling spares early breast cancer patients from adjuvant therapy: derived and validated in two population-based cohorts. Breast Cancer Res 7, R953-964.
Pelham, H. R. (1982). A regulatory upstream promoter element in the Drosophila hsp 70 heat-shock gene. Cell 30, 517-528.
Ritossa, F. (1962). A new puffing pattern induced by temperature shock and DNP in Drosophila. Experimentia 18, 571-573.
Rokavec, M., Wu, W., and Luo, J. L. (2012). IL6-Mediated Suppression of miR-200c Directs Constitutive Activation of Inflammatory Signaling Circuit Driving Transformation and Tumorigenesis. Mol Cell 45, 777-789.
Sakurai, H., and Enoki, Y. (2010). Novel aspects of heat shock factors: DNA recognition, chromatin modulation and gene expression. FEBS J 277, 4140-4149.
Santagata, S., Hu, R., Lin, N. U., Mendillo, M. L., Collins, L. C., Hankinson, S. E., Schnitt, S. J., Whitesell, L., Tamimi, R. M., Lindquist, S., et al. (2011). High levels of nuclear heat-shock factor 1 (HSF1) are associated with poor prognosis in breast cancer. Proc Natl Acad Sci USA 108, 18378-18383.
Santagata, S., Xu, Y. M., Wijeratne, E. M., Kontnik, R., Rooney, C., Perley, C. C., Kwon, H., Clardy, J., Kesari, S., Whitesell, L., et al. (2012). Using the heat-shock response to discover anticancer compounds that target protein homeostasis. ACS Chem Biol 7, 340-349.
Scott, K. L., Nogueira, C., Heffernan, T. P., van Doom, R., Dhakal, S., Hanna, J. A., Min, C., Jaskelioff, M., Xiao, Y., Wu, C. J., et al. (2011). Proinvasion metastasis drivers in early-stage melanoma are oncogenes. Cancer Cell 20, 92-103.
Shamovsky, I., and Nudler, E. (2008). New insights into the mechanism of heat shock response activation. Cell Mol Life Sci 65, 855-861.
Shimizu, H., Saliba, D., Wallace, M., Finlan, L., Langridge-Smith, P. R., and Hupp, T. R. (2006). Destabilizing missense mutations in the tumour suppressor protein p53 enhance its ubiquitination in vitro and in vivo. Biochem J 397, 355-367.
Silvera, D., Formenti, S. C., and Schneider, R. J. (2010). Translational control in cancer. Nat Rev Cancer 10, 254-266.
Solimini, N. L., Luo, J., and Elledge, S. J. (2007). Non-oncogene addiction and the stress phenotype of cancer cells. Cell 130, 986-988.
Soule, H. D., Vazguez, J., Long, A., Albert, S., and Brennan, M. (1973). A human cell line from a pleural effusion derived from a breast carcinoma. J Natl Cancer Inst 51, 1409-1416.
Stanhill, A., Levin, V., Hendel, A., Shachar, I., Kazanov, D., Arber, N., Kaminski, N., and Engelberg, D. (2006). Ha-ras(val12) induces HSP70b transcription via the HSE/HSF1 system, but HSP70b expression is suppressed in Ha-ras(val12)-transformed cells. Oncogene 25, 1485-1495.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Tamimi, R. M., Baer, H. J., Marotti, J., Galan, M., Galaburda, L., Fu, Y., Deitz, A. C., Connolly, J. L., Schnitt, S. J., Colditz, G. A., et al. (2008). Comparison of molecular phenotypes of ductal carcinoma in situ and invasive breast cancer. Breast Cancer Res 10, R67.
Tang, Y. C., Williams, B. R., Siegel, J. J., and Amon, A. (2011). Identification of aneuploidy-selective antiproliferation compounds. Cell 144, 499-512.
van 't Veer, L. J., Dai, H., van de Vijver, M. J., He, Y. D., Hart, A. A., Mao, M., Peterse, H. L., van der Kooy, K., Marton, M. J., Witteveen, A. T., et al. (2002). Gene expression profiling predicts clinical outcome of breast cancer. Nature 415, 530-536.
van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. N Engl J Med 347, 1999-2009.
Vander Heiden, M. G., Cantley, L. C., and Thompson, C. B. (2009). Understanding the Warburg effect: the metabolic requirements of cell proliferation. Science 324, 1029-1033.
Venet, D., Dumont, J. E., and Detours, V. (2011). Most random gene expression signatures are significantly associated with breast cancer outcome. PLoS Comput Biol 7, e1002240.
Volovik, Y., Maman, M., Dubnikov, T., Bejerano-Sagie, M., Joyce, D., Kapernick, E. A., Cohen, E., and Dillin, A. (2012). Temporal requirements of heat shock factor-1 for longevity assurance. Aging Cell.
Wang, Y., Klijn, J. G., Zhang, Y., Sieuwerts, A. M., Look, M. P., Yang, F., Talantov, D., Timmermans, M., Meijer-van Gelder, M. E., Yu, J., et al. (2005). Gene-expression profiles to predict distant metastasis of lymph-node-negative primary breast cancer. Lancet 365, 671-679.
Whitesell, L., and Lindquist, S. L. (2005). HSP90 and the chaperoning of cancer. Nat Rev Cancer 5, 761-772.
Xiao, X., Zuo, X., Davis, A. A., McMillan, D. R., Curry, B. B., Richardson, J. A., and Benjamin, I. J. (1999). HSF1 is required for extra-embryonic development, postnatal growth and protection during inflammatory responses in mice. EMBO J 18, 5943-5952.
Zhao, Y., Liu, H., Liu, Z., Ding, Y., Ledoux, S. P., Wilson, G. L., Voellmy, R., Lin, Y., Lin, W., Nahta, R., et al. (2011). Overcoming Trastuzumab Resistance in Breast Cancer by Targeting Dysregulated Glucose Metabolism. Cancer Res 71, 4585-4597.
Zhao, Y. H., Zhou, M., Liu, H., Ding, Y., Khong, H. T., Yu, D., Fodstad, O., and Tan, M. (2009). Upregulation of lactate dehydrogenase A by ErbB2 through heat shock factor 1 promotes breast cancer cell glycolysis and growth. Oncogene 28, 3689-3701.
Zhou, J. D., Luo, C. Q., Xie, H. Q., Nie, X. M., Zhao, Y. Z., Wang, S. H., Xu, Y., Pokharel, P. B., and Xu, D. (2008). Increased expression of heat shock protein 70 and heat shock factor 1 in chronic dermal ulcer tissues treated with laser-aided therapy. Chin Med J (Engl) 121, 1269-1273.

Those skilled in the art will recognize, or be able to ascertain using no more than routine experimentation, many equivalents to the specific embodiments of the invention described herein. The scope of the present invention is not intended to be limited to the Description or the details set forth therein. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). For example, any claim that is dependent on another claim can be modified to include one or more element(s), feature(s), or limitation(s) found in any other claim, e.g., any other claim that is dependent on the same base claim. Any one or more claims can be modified to explicitly exclude any one or more embodiment(s), element(s), feature(s), etc. For example, any particular type of tumor, tumor characteristic, test agent, candidate modulator, therapeutic agent, gene, set of genes, or combinations thereof can be excluded from any one or more claims.

It should be understood that (i) any method of classification, assessment, diagnosis, prognosis, treatment-specific prediction, treatment selection, treatment, etc., can include a step of providing a sample, e.g., a sample obtained from a subject in need of classification, assessment, diagnosis, prognosis, treatment-specific prediction, treatment selection, or treatment for cancer, e.g., a tumor sample obtained from the subject; (ii) any method of classification, assessment, diagnosis, prognosis, treatment-specific prediction, treatment selection, treatment, etc., can include a step of providing a subject in need of classification, assessment, diagnosis, prognosis, treatment-specific prediction, treatment selection, or treatment for cancer.

Where the claims recite a method, certain aspects of the invention provide a product, e.g., a kit or composition, suitable for performing the method.

Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. It will be understood that any description or exemplification of a term anywhere herein may be applied wherever such term appears herein (e.g., in any aspect or embodiment in which such term is relevant) unless indicated or clearly evident otherwise.

TABLE T1
Summary of ChIP-seq experiments

| | | | Heat- | | Total | Total | Count | Total | |
|---|---|---|---|---|---|---|---|---|---|
| | | | Shock | | Target | Background | Threshold | ChIP | Total Bound |
| Sample | | | (1 H, | ChIP | ChIP-Seq | (Rabbit IGG) | for ChIP | Enriched | Genes |
| Name | Replicate | Description | 42° C.) | Target | Reads | Reads | Enrichment | Regions | (RefSeq) |
| HMLER | R1 | Cell line | NO | HSF1 | 9533860 | 7423815 | 15 | 90 | 104 |
| BPLER | R1 | Cell line | NO | HSF1 | 8335254 | 10210111 | 14 | 1121 | 1274 |
| HME | R1 | Cell line | NO | HSF1 | 7871323 | 9620458 | 14 | 130 | 98 |
| BPE | R1 | Cell line | NO | HSF1 | 7666666 | 5532855 | 14 | 199 | 146 |
| HME | R1 | Cell line | YES | HSF1 | 4430889 | 4496512 | 14 | 1286 | 1130 |
| BPE | R1 | Cell line | YES | HSF1 | 5787581 | 3917571 | 12 | 1990 | 1494 |
| MCF10A | R1 | Cell line | NO | HSF1 | 16525555 | 9343984 | 18 | 359 | 355 |
| MCF10A | R2 | Cell line | NO | HSF1 | 7926575 | 9343984 | 14 | 35 | 45 |
| NCI1703 | R1 | Cell line | NO | HSF1 | 13750918 | 18449639 | 16 | 237 | 267 |
| NCI1703 | R2 | Cell line | NO | HSF1 | 15114498 | 18449639 | 17 | 26 | 38 |
| ZR75-1 | R1 | Cell line | NO | HSF1 | 13316786 | 13802906 | 16 | 190 | 235 |
| ZR75-1 | R2 | Cell line | NO | HSF1 | 17684812 | 13802906 | 18 | 250 | 305 |
| SW620 | R1 | Cell line | NO | HSF1 | 15331132 | 12899705 | 17 | 70 | 87 |
| SW620 | R2 | Cell line | NO | HSF1 | 16076936 | 12899705 | 17 | 50 | 44 |
| HCT15 | R1 | Cell line | NO | HSF1 | 11291744 | 8062691 | 15 | 444 | 588 |
| HCT15 | R2 | Cell line | NO | HSF1 | 9397580 | 8062691 | 14 | 168 | 217 |
| HT29 | R1 | Cell line | NO | HSF1 | 13715830 | 6685914 | 16 | 288 | 301 |
| HT29 | R2 | Cell line | NO | HSF1 | 13934563 | 6685914 | 17 | 506 | 620 |
| MCF7 | R1 | Cell line | NO | HSF1 | 10616586 | 10602750 | 15 | 51 | 46 |
| MCF7 | R2 | Cell line | NO | HSF1 | 10529277 | 10602750 | 15 | 233 | 249 |
| NCIH441 | R1 | Cell line | NO | HSF1 | 5145668 | 9558029 | 12 | 408 | 411 |
| NCIH441 | R2 | Cell line | NO | HSF1 | 7517421 | 9558029 | 13 | 914 | 918 |
| SKBR3 | R1 | Cell line | NO | HSF1 | 7242936 | 8920688 | 13 | 856 | 694 |
| SKBR3 | R2 | Cell line | NO | HSF1 | 7625838 | 8920688 | 14 | 1023 | 852 |
| NCIH838 | R1 | Cell line | NO | HSF1 | 17105568 | 12505419 | 18 | 2419 | 2472 |
| NCIH838 | R2 | Cell line | NO | HSF1 | 17935826 | 12505419 | 18 | 2401 | 2321 |
| BT20 | R1 | Cell line | NO | HSF1 | 5286464 | 13561259 | 12 | 1750 | 1736 |
| BT20 | R2 | Cell line | NO | HSF1 | 6935559 | 13561259 | 13 | 2396 | 2281 |
| HME | R2 | Cell line | YES | HSF1 | 10770106 | 7416762 | 15 | 3802 | 2762 |
| BPE | R2 | Cell line | YES | HSF1 | 10661149 | 7416762 | 15 | 2802 | 2106 |
| MCF10A | R1 | Cell line | YES | HSF1 | 8542755 | 7962816 | 14 | 1009 | 938 |
| MCF10A | R2 | Cell line | YES | HSF1 | 8427208 | 7962816 | 14 | 2876 | 2434 |
| BREAST-1 | R1 | Patient Tumor | NO | HSF1 | 16786625 | 18848070 | 18 | 166 | 194 |
| BREAST-1 | R2 | Patient Tumor | NO | HSF1 | 17977390 | 18848070 | 18 | 111 | 124 |
| BREAST-2 | R1 | Patient Tumor | NO | HSF1 | 15633433 | 14455453 | 17 | 457 | 439 |
| BREAST-2 | R2 | Patient Tumor | NO | HSF1 | 18861823 | 14455453 | 18 | 1068 | 939 |
| COLON-1 | R1 | Patient Tumor | NO | HSF1 | 14324235 | 13224764 | 17 | 217 | 256 |
| COLON-1 | R2 | Patient Tumor | NO | HSF1 | 12743139 | 13224764 | 16 | 349 | 379 |
| COLON-2 | R1 | Patient Tumor | NO | HSF1 | 8078461 | 7325580 | 14 | 175 | 191 |
| COLON-2 | R2 | Patient Tumor | NO | HSF1 | 4598942 | 7325580 | 12 | 118 | 103 |

TABLE T2A
BPLER Only: Gene Set Enrichment Analysis results

| Collection(s): | C1, CP:KEGG, CP:REACTOME, MF |
|---|---|
| # genesets in collections: | 2163 |
| # genes in comparison (n): | 481 |
| # genes in collections (N): | 25278 |

| | | # Genes in | # Genes in | | |
|---|---|---|---|---|---|
| | | Gene Set | Overlap | | |
| Gene Set Name | Description | (K) | (k) | k/K | p value |
| chr8q24 | Genes in cytogenetic band chr8q24 | 182 | 29 | 0.1593 | 0.00E+00 |
| chr11q13 | Genes in cytogenetic band | 292 | 20 | 0.0685 | 9.57E−07 |
| | chr11q13 | | | | |
| REACTOME_GENE_EXPRESSION | Genes involved in Gene Expression | 425 | 24 | 0.0565 | 2.54E−06 |
| REACTOME_TRANSLATION | Genes involved in Translation | 120 | 11 | 0.0917 | 1.90E−05 |
| REACTOME_INFLUENZA_VIRAL_RNA_{—} | Genes involved in Influenza Viral | 100 | 10 | 0.1 | 2.13E−05 |
| TRANSCRIPTION_AND_REPLICATION | RNA Transcription and Replication | | | | |
| BIOPOLYMER_METABOLIC_PROCESS | Genes annotated by the GO term | 1633 | 55 | 0.0337 | 2.87E−05 |
| | GO:0043283. The chemical | | | | |
| | reactions and pathways involving | | | | |
| | biopolymers, long, repeating chains | | | | |
| | of monomers found in nature e.g. | | | | |
| | polysaccharides and proteins. | | | | |
| RNA_BINDING | Genes annotated by the GO term | 239 | 15 | 0.0628 | 5.93E−05 |
| | GO:0003723. Interacting | | | | |
| | selectively with an RNA molecule or | | | | |
| | a portion thereof. | | | | |
| REACTOME_INFLUENZA_LIFE_CYCLE | Genes involved in Influenza Life | 137 | 11 | 0.0803 | 6.50E−05 |
| | Cycle | | | | |
| chr7p22 | Genes in cytogenetic band chr7p22 | 74 | 8 | 0.1081 | 8.14E−05 |
| REACTOME_GTP_HYDROLYSIS_AND_{—} | Genes involved in GTP hydrolysis | 106 | 9 | 0.0849 | 1.95E−04 |
| JOINING_OF_THE_60S_RIBOSOMAL_{—} | and joining of the 60S ribosomal | | | | |
| SUBUNIT | subunit | | | | |
| REACTOME_PEPTIDE_CHAIN_ELON- | Genes involved in Peptide chain | 84 | 8 | 0.0952 | 2.00E−04 |
| GATION | elongation | | | | |
| REACTOME_VIRAL_MRNA_TRANSLA- | Genes involved in Viral mRNA | 84 | 8 | 0.0952 | 2.00E−04 |
| TION | Translation | | | | |
| PROTEIN_METABOLIC_PROCESS | Genes annotated by the GO term | 1199 | 41 | 0.0342 | 2.29E−04 |
| | GO:0019538. The chemical | | | | |
| | reactions and pathways involving a | | | | |
| | specific protein, rather than of | | | | |
| | proteins in general. Includes | | | | |
| | protein modification. | | | | |
| chr16q22 | Genes in cytogenetic band | 134 | 10 | 0.0746 | 2.53E−04 |
| | chr16q22 | | | | |
| REACTOME_METABOLISM_OF_PRO- | Genes involved in Metabolism of | 215 | 13 | 0.0605 | 2.62E−04 |
| TEINS | proteins | | | | |
| KEGG_RIBOSOME | Ribosome | 88 | 8 | 0.0909 | 2.75E−04 |
| REACTOME_RNA_POLYMERASE_III_{—} | Genes involved in RNA Polymerase | 34 | 5 | 0.1471 | 4.31E−04 |
| TRANSCRIPTION | III Transcription | | | | |
| REACTOME_METABOLISM_OF_CARBO- | Genes involved in Metabolism of | 119 | 9 | 0.0756 | 4.62E−04 |
| HYDRATES | carbohydrates | | | | |
| REACTOME_FORMATION_OF_A_POOL_{—} | Genes involved in Formation of a | 95 | 8 | 0.0842 | 4.64E−04 |
| OF_FREE_40S_SUBUNITS | pool of free 40S subunits | | | | |
| KEGG_FOCAL_ADHESION | Focal adhesion | 201 | 12 | 0.0597 | 5.00E−04 |

TABLE T2B
BPLER and Heat-Shock: Gene Set Enrichment Analysis results

| Collection(s): | C1, CP:KEGG, CP:REACTOME, MF |
|---|---|
| # genesets in collections: | 1338 |
| # genes in comparison (n): | 482 |
| # genes in collections (N): | 25227 |

| | | # Genes | # Genes in | | |
|---|---|---|---|---|---|
| | | in Gene | Overlap | | |
| Gene Set Name | Description | Set (K) | (k) | k/K | p value |
| PROTEIN_FOLDING | Genes annotated by the GO term | 56 | 11 | 0.1964 | 2.05E−10 |
| | GO:0006457. The process of assisting in | | | | |
| | the covalent and noncovalent assembly of | | | | |
| | single chain polypeptides or multisubunit | | | | |
| | complexes into the correct tertiary | | | | |
| | structure. | | | | |
| RESPONSE_TO_BIOTIC_STIM- | Genes annotated by the GO term | 117 | 13 | 0.1111 | 7.09E−09 |
| ULUS | GO:0009607. A change in state or activity | | | | |
| | of a cell or an organism (in terms of | | | | |
| | movement, secretion, enzyme production, | | | | |
| | gene expression, etc.) as a result of a | | | | |
| | biotic stimulus, a stimulus caused or | | | | |
| | produced by a living organism. | | | | |
| UNFOLDED_PROTEIN_BIND- | Genes annotated by the GO term | 41 | 7 | 0.1707 | 1.18E−06 |
| ING | GO:0051082. Interacting selectively with | | | | |
| | an unfolded protein. | | | | |
| PROTEIN_METABOLIC_PRO- | Genes annotated by the GO term | 1199 | 37 | 0.0309 | 2.85E−06 |
| CESS | GO:0019538. The chemical reactions and | | | | |
| | pathways involving a specific protein, | | | | |
| | rather than of proteins in general. | | | | |
| | Includes protein modification. | | | | |
| CELLULAR_PROTEIN_META- | Genes annotated by the GO term | 1086 | 34 | 0.0313 | 5.45E−06 |
| BOLIC_PROCESS | GO:0044267. The chemical reactions and | | | | |
| | pathways involving a specific protein, | | | | |
| | rather than of proteins in general, | | | | |
| | occurring at the level of an individual cell. | | | | |
| | Includes protein modification. | | | | |
| CELLULAR_MACROMOLECULE_{—} | Genes annotated by the GO term | 1100 | 34 | 0.0309 | 7.14E−06 |
| METABOLIC_PROCESS | GO:0044260. The chemical reactions and | | | | |
| | pathways involving macromolecules, large | | | | |
| | molecules inciuding proteins, nucleic acids | | | | |
| | and carbohydrates, as carried out by | | | | |
| | individual cells. | | | | |
| REACTOME_FORMATION_OF_{—} | Genes involved in Formation of tubulin | 22 | 5 | 0.2273 | 9.60E−06 |
| TUBULIN_FOLDING_INTER- | folding intermediates by CCT/TriC | | | | |
| MEDIATES_BY_CCT_TRIC | | | | | |
| CHAPERONE_BINDING | Genes annotated by the GO term | 12 | 4 | 0.3333 | 1.50E−05 |
| | GO:0051087. Interacting selectively with | | | | |
| | a chaperone protein, a class of proteins | | | | |
| | that bind to nascent or unfolded | | | | |
| | polypeptides and ensure correct folding or | | | | |
| | transport. | | | | |
| REACTOME_CELL_DEATH_SIG- | Genes involved in Cell death signalling via | 61 | 7 | 0.1148 | 1.82E−05 |
| NALLING_VIA_NRAGE_NRIF_{—} | NRAGE, NRIF and NADE | | | | |
| AND_NADE | | | | | |
| NITROGEN_COMPOUND_BIO- | Genes annotated by the GO term | 25 | 5 | 0.2 | 1.87E−05 |
| SYNTHETIC_PROCESS | GO:0044271. The chemical reactions and | | | | |
| | pathways resulting in the formation of | | | | |
| | organic and inorganic nitrogenous | | | | |
| | compounds. | | | | |
| POSITIVE_REGULATION_OF_{—} | Genes annotated by the GO term | 645 | 23 | 0.0357 | 2.69E−05 |
| CELLULAR_PROCESS | GO:0048522. Any process that activates | | | | |
| | or increases the frequency, rate or extent | | | | |
| | of cellular processes, those that are | | | | |
| | carried out at the cellular level, but are | | | | |
| | not necessarily restricted to a single cell. | | | | |
| | For example, cell communication occurs | | | | |
| | among more than one cell, but occurs at | | | | |
| | the cellular level. | | | | |
| ENZYME_REGULATGR_ACTIV- | Genes annotated by the GO term | 314 | 15 | 0.0478 | 2.78E−05 |
| ITY | GO:0030234. Modulates the activity of an | | | | |
| | enzyme. | | | | |
| REACTOME_PREFOLDIN_ME- | Genes involved in Prefoldin mediated | 28 | 5 | 0.1786 | 3.35E−05 |
| DIATED_TRANSFER_OF_SUB- | transfer of substrate to CCT/TriC | | | | |
| STRATE_TO_CCT_TRIC | | | | | |
| REACTOME_ASSOCIATION | Genes involved in Association of TriC/CCT | 29 | 5 | 0.1724 | 4.00E−05 |
| OF_TRIC_CCT_WITH_TARGET_{—} | with target proteins during biosynthesis | | | | |
| PROTEINS_DURING_BIO- | | | | | |
| SYNTHESIS | | | | | |
| chr21p11 | Genes in cytogenetic band chr21p11 | 6 | 3 | 0.5 | 4.76E−05 |
| REACTOME_FORMATION_OF_{—} | Genes involved in Formation of Platelet | 186 | 11 | 0.0591 | 4.94E−05 |
| PLATELET_PLUG | plug | | | | |
| POSITIVE_REGULATION_OF_{—} | Genes annotated by the GO term | 222 | 12 | 0.0541 | 5.44E−05 |
| CELLULAR_METABOLIC_PRO- | GO:0031325. Any process that activates | | | | |
| CESS | or increases the frequency, rate or extent | | | | |
| | of the chemical reactions and pathways | | | | |
| | by which individual cells transform | | | | |
| | chemical substances. | | | | |
| POSITIVE_REGULATION_OF_{—} | Genes annotated by the GO term | 686 | 23 | 0.0335 | 6.88E−05 |
| BIOLOGICAL_PROCESS | GO:0048518. Any process that activates | | | | |
| | or increases the frequency, rate or extent | | | | |
| | of a biological process. Biological | | | | |
| | processes are regulated by many means; | | | | |
| | examples include the control of gene | | | | |
| | expression, protein modification or | | | | |
| | interaction with a protein or substrate | | | | |
| | molecule. | | | | |
| POSITIVE_REGULATION_OF_{—} | Genes annotated by the GO term | 229 | 12 | 0.0524 | 7.32E−05 |
| METABOLIC_PROCESS | GO:0009893. Any process that activates | | | | |
| | or increases the frequency, rate or extent | | | | |
| | of the chemical reactions and pathways | | | | |
| | within a cell or an organism. | | | | |
| KEGG_NON_SMALL_CELL_{—} | Non-small cell lung cancer | 54 | 6 | 0.1111 | 8.77E−05 |
| LUNG_CANCER | | | | | |

TABLE T3

| | | | | | | | | | | HMLER |
|---|---|---|---|---|---|---|---|---|---|---|
| | Representative | | | | | | | | | (GFP vs |
| Probe Set ID | Public ID | Gene Symbol | Entrez Gene | SCR_HMLER_A | SCR_HMLER_B | GFP_HMLER_A | GFP_HMLER_B | ha6_HMLER_A | ha6_HMLER_B | SCR) |
| 117_at | X51757 | HSPA6 | 3310 | 3.076 | 3.1 | 3.02 | 2.99 | 2.92 | 2.79 | −0.082 |
| 121_at | X69699 | RAX8 | 7849 | 5.199 | 5.18 | 5.22 | 5.21 | 5.11 | 5.028 | 0.0285 |
| 1487_at | L38487 | ESRRA | 2101 | 5.562 | 5.61 | 5.53 | 5.7 | 5.71 | 5.493 | 0.0301 |
| 200002_at | NM_007209 | RPL35 | 11224 | 11.58 | 11.7 | 11.5 | 11.6 | 11.6 | 11.57 | −0.1 |
| 200017_at | NM_002954 | RPS27A /// UBB /// | 6233 /// 7314 /// | 12.4 | 12.4 | 12.4 | 12.3 | 12.3 | 12.35 | −0.063 |
| | | UBC | 7316 | | | | | | | |
| 200019_s_at | NM_001997 | FAU | 2197 | 12.07 | 12.1 | 12.1 | 12.2 | 12.1 | 12.14 | 0.0677 |
| 200022_at | NM_000979 | RPL18 | 6141 | 12.44 | 12.5 | 12.5 | 12.4 | 12.3 | 12.42 | −0.039 |
| 200024_at | NM_001009 | RPSS | 6193 | 12.01 | 12.1 | 12 | 12.1 | 12 | 12 | 0.0145 |
| 200037_s_at | NM_016587 | CBX3 /// LOC653972 | 11335 /// 653972 | 10.67 | 10.7 | 10.5 | 10.6 | 10.1 | 10.1 | −0.148 |
| 200049_at | NM_007067 | MYST2 | 11143 | 7.44 | 7.39 | 7.18 | 7.19 | 7.04 | 7.228 | −0.229 |
| 200064_at | AF275719 | HSP90AB1 | 3326 | 10.63 | 10.6 | 10.6 | 10.6 | 10.2 | 10.39 | −0.04 |
| 200067_x_at | AL078595 | SNX3 | 8724 | 10.99 | 11 | 10.9 | 10.8 | 11 | 10.99 | −0.126 |
| 200601_at | U48734 | ACTN4 | 81 | 6.736 | 6.74 | 6.69 | 6.95 | 6.76 | 6.792 | 0.0786 |
| 200602_at | NM_000484 | APP | 351 | 9.593 | 9.59 | 9.52 | 9.51 | 9.37 | 9.59 | −0.076 |
| 200618_at | NM_006148 | LASP1 | 3927 | 8.182 | 8.17 | 8.26 | 8.17 | 8.2 | 8.07 | 0.0358 |
| 200622_x_at | AV685208 | CALM1 /// C4LM2 /// | 801 /// 805 /// 808 | 6.674 | 6.68 | 6.53 | 6.5 | 6.74 | 6.702 | −0.164 |
| | | CALM3 | | | | | | | | |
| 200623_s_at | NM_005184 | CALM1 /// CALM2 /// | 801 /// 805 /// 808 | 5.178 | 5.04 | 4.91 | 5.13 | 5.64 | 5.288 | −0.09 |
| | | CALM3 | | | | | | | | |
| 200627_at | BC003005 | PTGES3 | 10728 | 11.42 | 11.4 | 11.4 | 11.4 | 11.3 | 11.34 | −0.003 |
| 200632_s_at | NM_006096 | NDRG1 | 10397 | 10.71 | 10.7 | 10.7 | 10.7 | 10.4 | 10.51 | −0.025 |
| 200633_at | NM_018955 | RPS27A /// UBB /// | 6233 /// 7314 /// | 12.63 | 12.6 | 12.6 | 12.6 | 12.7 | 12.67 | −0.021 |
| | | UBC | 7316 | | | | | | | |
| 200653_s_at | M27319 | CALM1 /// CALM2 /// | 801 /// 805 /// 808 | 9.55 | 9.46 | 9.49 | 9.44 | 9.29 | 9.459 | −0.041 |
| | | CALM3 | | | | | | | | |
| 200655_s_at | NM_006888 | CALM1 /// CALM2 /// | 801 /// 805 /// 808 | 9.271 | 9.24 | 9.2 | 9.29 | 9.33 | 9.354 | −0.012 |
| | | CALM3 | | | | | | | | |
| 200664_s_at | BG537255 | DNAJB1 | 3337 | 7.421 | 7.6 | 7.54 | 7.36 | 7.33 | 7.36 | −0.058 |
| 200666_s_at | NM_006145 | DNAJB1 | 3337 | 7.766 | 7.71 | 7.71 | 7.76 | 7.67 | 7.668 | −0.001 |
| 200667_at | BF448062 | UBE2D3 | 7323 | 9.271 | 9.21 | 9.29 | 9.37 | 9.18 | 9.148 | 0.0911 |
| 200668_s_at | BC003395 | UBE2D3 | 7323 | 10.19 | 10.2 | 10.2 | 10.1 | 10.1 | 10.1 | −0.031 |
| 200669_s_at | NM_003340 | UBE2D3 | 7323 | 9.467 | 9.42 | 9.42 | 9.45 | 9.55 | 9.399 | −0.008 |
| 200687_s_at | NM_012426 | SF3B3 | 23450 | 7.296 | 7.3 | 7.48 | 7.31 | 7.38 | 7.225 | 0.0985 |
| 200688_at | D13642 | SF3B3 | 23450 | 4.091 | 3.84 | 4 | 3.89 | 4.07 | 3.681 | −0.019 |
| 200689_x_at | NM_001404 | EEF1G | 1937 | 12.36 | 12.3 | 12.2 | 12.3 | 12.1 | 12.27 | −0.073 |
| 200696_s_at | NM_000177 | GSN | 2934 | 7.451 | 7.59 | 7.57 | 7.63 | 7.6 | 7.721 | 0.0787 |
| 200707_at | NM_002743 | PRXCSH | 5589 | 6.933 | 6.82 | 6.9 | 7 | 6.84 | 6.815 | 0.0731 |
| 200737_at | NM_000791 | PGK1 | 5230 | 8.16 | 8.23 | 8.1 | 8.14 | 7.71 | 7.99 | −0.077 |
| 200738_s_at | NM_000291 | PGK1 | 5230 | 10.82 | 10.8 | 10.8 | 10.7 | 10.8 | 10.72 | −0.066 |
| 200753_x_at | BE866585 | SFRS2 | 6427 | 8.373 | 8.27 | 8.12 | 8.23 | 8.05 | 8.06 | −0.145 |
| 200754_x_at | NM_003016 | SF952 | 6427 | 9.995 | 9.87 | 10.1 | 10 | 10.1 | 10.13 | 0.0974 |
| 200768_s_at | BC001686 | MAT2A | 4144 | 8.971 | 8.97 | 9.05 | 9.09 | 8.94 | 8.961 | 0.1036 |
| 200769_s_at | NM_005911 | MAT2A | 4144 | 5.833 | 6.17 | 5.93 | 5.73 | 5.71 | 5.791 | −0.174 |
| 200806_s_at | BE256479 | HSPD1 | 3329 | 11.37 | 11.3 | 11.5 | 11.3 | 11.3 | 11.23 | 0.0472 |
| 200807_s_at | NM_002156 | HSPD1 | 3329 | 11.7 | 11.7 | 11.6 | 11.6 | 11.8 | 11.72 | −0.092 |
| 200812_at | NM_006429 | CCT7 | 10574 | 9.318 | 9.3 | 9.14 | 9.32 | 9.4 | 9.341 | −0.073 |
| 200823_x_at | NM_000992 | LOC100131713 /// | 100131713 /// | 11.87 | 11.9 | 11.7 | 11.8 | 11.4 | 11.64 | −0.101 |
| | | RPL29 /// RPL29P4 | 387101 /// 6159 | | | | | | | |
| 200828_s_at | BE871379 | ZNF207 | 7756 | 9.622 | 9.63 | 9.56 | 9.5 | 9.44 | 9.603 | −0.095 |
| 200829_x_at | NM_003457 | ZNF207 | 7756 | 9.592 | 9.51 | 9.5 | 9.42 | 9.58 | 9.487 | −0.09 |
| 200847_s_at | NM_016127 | TMEM66 | 51669 | 11.15 | 11.1 | 11 | 11 | 10.7 | 10.81 | −0.097 |
| 200854_at | AB028970 | NCOR1 | 9611 | 6.815 | 6.83 | 6.78 | 6.9 | 7.14 | 6.871 | 0.0186 |
| 200857_s_at | NM_006311 | NCOR1 | 9611 | 6.775 | 6.71 | 6.98 | 6.85 | 6.99 | 9.649 | 0.1738 |
| 200873_s_at | NM_006585 | CCT8 | 10694 | 11.37 | 11.4 | 11.3 | 11.4 | 11.4 | 11.44 | 0.0031 |
| 200877_at | NM_006430 | CCT4 | 10575 | 11.5 | 11.5 | 11.5 | 11.5 | 11.4 | 11.48 | 0.0018 |
| 200880_at | AL534104 | DNAJA1 | 3301 | 7.932 | 7.93 | 7.93 | 7.81 | 7.81 | 7.849 | −0.055 |
| 200881_s_at | NM_001539 | DNAJA1 | 3301 | 9.69 | 9.8 | 9.72 | 9.75 | 9.41 | 9.435 | −0.011 |
| 200892_s_at | BC000451 | SFRS10 | 6434 | 8.744 | 8.68 | 8.6 | 8.63 | 8.73 | 8.667 | −0.095 |
| 200893_at | NM_004593 | SFRS10 | 6434 | 10.9 | 10.8 | 10.8 | 10.8 | 11 | 11 | −0.05 |
| 200894_s_at | AA894574 | FKBP4 | 2288 | 6.85 | 6.94 | 6.83 | 6.78 | 6.43 | 6.504 | −0.09 |
| 290895_s_at | NM_002014 | FXBP4 | 2288 | 7.3 | 7.48 | 7.43 | 7.34 | 7.19 | 7.209 | −0.004 |
| 200896_x_at | NM_004494 | HDGF | 3068 | 9.666 | 9.59 | 9.59 | 9.6 | 9.56 | 9.474 | −0.035 |
| 200910_at | NM_005998 | CCT3 | 7203 | 9.516 | 9.42 | 9.35 | 9.4 | 9.2 | 9.417 | −0.094 |
| 200912_s_at | NM_001967 | EIF4A2 | 1974 | 12.08 | 12.1 | 12.1 | 12.1 | 12 | 11.99 | −0.013 |
| 200936_at | NM_000973 | RPL8 | 6132 | 13.01 | 13 | 13 | 13 | 13 | 13.02 | 0.0231 |
| 200965_s_at | NM_006720 | ABLIM1 | 3983 | 5.58 | 5.58 | 5.49 | 5.52 | 5.87 | 5.733 | −0.074 |
| 200983_x_at | BF983379 | CD59 | 966 | 11.32 | 11.4 | 11.4 | 11.4 | 11.1 | 11.1 | −0.004 |
| 200984_s_at | X16447 | CD59 | 966 | 10.2 | 10.3 | 10.4 | 10.4 | 9.83 | 9.971 | 0.123 |
| 200985_s_at | NM_000611 | CD59 | 966 | 10.18 | 10.2 | 10.3 | 10.3 | 10.1 | 10.17 | 0.1063 |
| 201023_at | NM_005642 | TAF7 | 6879 | 8.136 | 8.32 | 8.33 | 8.2 | 8.37 | 8.36 | 0.0332 |
| 201066_at | NM_001916 | CYC1 | 1537 | 9.032 | 8.99 | 9.03 | 9.15 | 9.22 | 9.25 | 0.0798 |
| 201079_at | NM_004710 | SYNGR2 | 9144 | 6.627 | 6.71 | 6.6 | 6.46 | 6.52 | 6.653 | −0.139 |
| 201091_s_at | BE748755 | CBX3 /// LOC653972 | 11335 /// 653972 | 1592 | 9.68 | 9.45 | 9.73 | 9.24 | 9.469 | −0.045 |
| 201129_at | NM_006276 | SFRS7 | 6432 | 7.782 | 7.79 | 7.78 | 7.81 | 7.99 | 7.923 | 0.0069 |
| 201132_at | NM_019597 | HNRNPH2 | 3188 | 7.072 | 7.16 | 7.06 | 7.09 | 6.66 | 6.943 | −0.044 |
| 201140_s_at | NM_004583 | RAB5C | 5878 | 8.013 | 7.64 | 8.16 | 8.1 | 7.62 | 7.499 | 0.3097 |
| 201156_s_at | AF141304 | RAB5C | 5878 | 8.147 | 8.01 | 8.09 | 8.2 | 7.53 | 7.871 | 0.0686 |
| 201162_at | NM_001553 | IGFBP7 | 3490 | 8.38 | 8.45 | 8.33 | 8.54 | 8.03 | 7.881 | 0.0212 |
| 201163_s_at | NM_001553 | IGFBP7 | 3490 | 9.943 | 9.97 | 9.8 | 10 | 9.4 | 9.384 | −0.051 |
| 201173_x_at | NM_006600 | NUDC | 10726 | 8.788 | 8.76 | 8.79 | 8.76 | 8.84 | 8.856 | 0.0014 |
| 201182_s_at | AI761771 | CHD4 | 1108 | 6.514 | 6.62 | 6.66 | 6.52 | 6.63 | 6.718 | 0.0207 |
| 201183_s_at | AI613273 | CHD4 | 1108 | 7.036 | 7.24 | 7.25 | 7.33 | 7.01 | 7.204 | 0.1508 |
| 201184_s_at | NM_001273 | CHD4 | 1108 | 6.814 | 6.9 | 6.73 | 6.79 | 6.68 | 6.616 | −0.098 |
| 201194_at | NM_003009 | SEPW1 | 6415 | 8.989 | 9.03 | 9.02 | 8.98 | 9.02 | 8.874 | −0.008 |
| 201218_at | N23018 | CTBP2 | 1488 | 9.662 | 9.62 | 9.6 | 9.46 | 9.4 | 9.296 | −0.112 |
| 201219_at | AW269836 | CTBP2 | 1488 | 6.9 | 6.71 | 6.82 | 6.62 | 6.86 | 6.693 | −0.087 |
| 201220_x_at | NM_001329 | CTBP2 | 1488 | 10.1 | 9.92 | 10 | 10 | 9.98 | 10.01 | 0.0015 |
| 201249_at | AI091047 | SLC2A1 | 6513 | 4.383 | 4.35 | 4.23 | 4.05 | 4.18 | 3.971 | −0.226 |
| 201250_s_at | NM_006516 | SLC2A1 | 6513 | 7.425 | 7.31 | 7.15 | 7.38 | 7.58 | 7.436 | −0.104 |
| 201269_s_at | AB028991 | NUDCD3 | 23386 | 3.248 | 3.21 | 3.17 | 3.01 | 2.98 | 3.207 | −0.144 |
| 201270_x_at | NM_015332 | NUDCD3 | 23386 | 7.507 | 7.57 | 7.71 | 7.57 | 7.65 | 7.581 | 0.105 |
| 201301_s_at | BC000182 | ANXA4 | 307 | 9.351 | 9.43 | 9.22 | 9.24 | 9.31 | 9.326 | −0.16 |
| 201302_at | NM_001153 | ANXA4 | 307 | 8.068 | 8.02 | 8 | 7.95 | 7.81 | 7.816 | −0.067 |
| 201326_at | BE737030 | CCT6A | 908 | 9.568 | 9.61 | 9.69 | 9.69 | 9.56 | 9.678 | 0.0975 |
| 201327_s_at | NM_001762 | CCT6A | 908 | 10.82 | 10.8 | 10.7 | 10.9 | 10.6 | 10.71 | 0.0054 |
| 201331_s_at | BC004973 | STAT6 | 6778 | 5.814 | 5.93 | 5.65 | 5.41 | 5.68 | 5.792 | −0.342 |
| 201332_s_at | NM_003153 | STAT6 | 6778 | 3.174 | 3.33 | 3.51 | 3.42 | 3.48 | 3.15 | 0.2133 |
| 201373_at | NM_000445 | PLEC1 | 5339 | 7.348 | 7.28 | 7.4 | 7.26 | 7.59 | 7.195 | 0.0117 |
| 201379_s_at | NM_003288 | TPD52L2 | 7165 | 7.644 | 7.47 | 7.7 | 7.63 | 7.49 | 7.409 | 0.1093 |
| 201381_x_at | AF057356 | CACYBP | 27101 | 10.13 | 10.2 | 10.1 | 10.2 | 10.1 | 10.06 | −0.044 |
| 201382_at | NM_014412 | CACYBP | 27101 | 3.277 | 3.74 | 3.35 | 3.44 | 3.53 | 3.49 | −0.114 |
| 201388_at | NM_002809 | PSMD3 | 5709 | 7.252 | 7.31 | 7.39 | 7.28 | 7.11 | 7.366 | 0.0563 |
| 201400_at | NM_002795 | PSMB3 | 5691 | 9.57 | 9.64 | 9.55 | 9.52 | 9.5 | 9.597 | −0.072 |
| 201401_s_at | M80776 | ADRBK1 | 156 | 3.999 | 4.07 | 3.87 | 4.06 | 3.97 | 3.681 | −0.069 |
| 201402_at | NM_001619 | ADRBK1 | 156 | 4.227 | 4.08 | 4.23 | 4.18 | 4.03 | 3.968 | 0.0493 |
| 201423_s_at | AL037208 | CUL4A | 8451 | 6.509 | 6.53 | 6.68 | 6.52 | 6.6 | 6.554 | 0.0815 |
| 201424_s_at | NM_003589 | CUL4A | 8451 | 7.113 | 7.07 | 7.16 | 7.19 | 7.25 | 7.048 | 0.0843 |
| 201491_at | NM_012111 | AHSA1 | 10598 | 8.546 | 8.61 | 8.51 | 8.45 | 8.37 | 8.517 | −0.097 |
| 201559_s_at | AF109196 | CLIC4 | 25932 | 7.908 | 8.06 | 7.83 | 7.82 | 7.53 | 7.619 | −0.159 |
| 201560_at | NM_013943 | CLIC4 | 25932 | 10.34 | 10.3 | 10.4 | 10.3 | 10.3 | 10.29 | −0.012 |
| 201564_s_at | NM_003088 | FSCN1 | 6624 | 5.766 | 5.71 | 5.95 | 6.09 | 6.19 | 5.957 | 0.2793 |
| 201578_at | NM_005397 | PODXL | 5420 | 4.093 | 3.88 | 4.04 | 3.9 | 4.13 | 3.947 | −0.014 |
| 201605_x_at | NM_004368 | CNN2 | 1265 | 4.444 | 4.42 | 4.63 | 4.56 | 4.27 | 4.656 | 0.1668 |
| 201621_at | NM_005380 | NBL1 | 4681 | 5.688 | 5.95 | 5.83 | 5.8 | 5.92 | 5.87 | −0.005 |
| 201623_s_at | BC000629 | DARS | 1615 | 9.853 | 9.88 | 9.85 | 9.86 | 9.97 | 9.887 | −0.006 |
| 201624_at | NM_001349 | DARS | 1615 | 7.046 | 7.01 | 6.83 | 7.06 | 7.29 | 7.169 | −0.086 |
| 201635_s_at | AI990766 | FXR1 | 8087 | 9.203 | 9.22 | 9.12 | 9.1 | 8.84 | 8.89 | −0.103 |
| 201636_at | BG025078 | FXR1 | 8087 | 8.239 | 8.34 | 8.26 | 8.27 | 8.33 | 8.246 | −0.026 |
| 201637_s_at | NM_005087 | FXR1 | 8087 | 10.19 | 10.2 | 10.1 | 10.2 | 9.97 | 10.01 | 0.0032 |
| 201638_s_at | BE676642 | CPSF1 | 29894 | 3.077 | 2.95 | 3.03 | 2.93 | 2.91 | 3.231 | −0.036 |
| 201639_s_at | NM_013291 | CPSF1 | 29894 | 6.393 | 6.38 | 6.49 | 6.28 | 6.45 | 6.491 | −0.005 |
| 201642_at | NM_005534 | IFNGR2 | 3460 | 7.592 | 7.83 | 7.76 | 7.86 | 7.69 | 7.522 | 0.0993 |
| 201643_x_at | NM_016604 | JMJD1B | 51780 | 5.535 | 5.55 | 5.52 | 5.61 | 5.82 | 5.565 | 0.211 |
| 201654_s_at | AI991033 | HSPG2 | 3339 | 2.846 | 3.09 | 2.82 | 3.02 | 3.05 | 2.807 | −0.046 |
| 201655_s_at | M85289 | HSPG2 | 3339 | 4.988 | 4.94 | 5.07 | 5.43 | 5.26 | 5.039 | 0.2858 |
| 201688_s_at | BG389015 | TPD52 | 7163 | 8.135 | 8.22 | 8.15 | 8.03 | 8.11 | 8.103 | −0.083 |
| 201689_s_at | BE974098 | TPD52 | 7163 | 8.455 | 8.37 | 8.33 | 8.44 | 8.22 | 8.313 | −0.028 |
| 201690_s_at | AA524023 | TPD52 | 7163 | 9.623 | 9.47 | 9.62 | 9.67 | 9.69 | 9.547 | 0.1008 |
| 201691_s_at | NM_005079 | TPD52 | 7163 | 3.524 | 3.61 | 3.43 | 3.34 | 3.7 | 3.467 | −0.183 |
| 201711_x_at | AI681120 | RANBP2 | 5903 | 6.812 | 6.9 | 6.92 | 6.84 | 6.78 | 6.687 | 0.0214 |
| 201712_s_at | NM_006267 | RANBP2 | 5903 | 4.644 | 4.98 | 5.1 | 4.76 | 4.78 | 4.934 | 0.1165 |
| 201713_s_at | D42063 | RANBP2 | 5903 | 6.938 | 6.93 | 6.96 | 6.93 | 6.73 | 6.917 | 0.0121 |
| 201717_at | NM_004927 | MRPL49 | 740 | 8.86 | 8.82 | 8.84 | 8.88 | 9.06 | 8.829 | 0.0163 |
| 201751_at | NM_014876 | JOSD1 | 9929 | 8.224 | 8.21 | 8.18 | 8.03 | 8.06 | 8.115 | −0.114 |
| 201772_at | NM_015878 | AZIN1 | 51582 | 9.351 | 9.43 | 9.4 | 9.34 | 9.17 | 9.292 | −0.025 |
| 201841_s_at | NM_001540 | HSPB1 | 3315 | 7.711 | 7.79 | 7.69 | 7.75 | 7.54 | 7.746 | −0.03 |
| 201842_s_at | AI826799 | EFEMP1 | 2202 | 8.157 | 8.14 | 8.14 | 8.16 | 8.04 | 8.177 | 0.0033 |
| 201843_s_at | NM_004105 | EFEMP1 | 2202 | 4.893 | 4.62 | 4.86 | 4.45 | 4.32 | 4.645 | −0.098 |
| 201853_s_at | NM_021873 | CDC258 | 994 | 6.836 | 6.9 | 6.91 | 6.9 | 6.89 | 6.821 | 0.0428 |
| 201913_s_at | NM_025233 | COASY | 80347 | 7.652 | 7.57 | 7.58 | 7.65 | 7.6 | 7.657 | 0.0053 |
| 201922_at | NM_014886 | TINP1 | 10412 | 10.53 | 10.5 | 10.5 | 10.5 | 10.7 | 10.63 | −0.029 |
| 201971_s_at | NM_001690 | ATP6V1A | 523 | 5.651 | 5.58 | 5.6 | 5.22 | 5.36 | 5.335 | −0.208 |
| 201972_at | AF113129 | ATP6V1A | 523 | 9.436 | 9.36 | 9.39 | 9.45 | 9.38 | 9.395 | 0.0226 |
| 201983_s_at | AW157070 | EGFR | 1956 | 8.863 | 8.81 | 8.87 | 8.9 | 8.9 | 8.881 | 0.0481 |
| 201984_s_at | NM_005228 | EGFR | 1956 | 6.816 | 6.79 | 6.94 | 6.9 | 6.92 | 6.814 | 0.1149 |
| 201994_at | NM_012286 | MORF4L2 | 9643 | 11.17 | 11.1 | 11.1 | 11 | 11 | 10.93 | −0.039 |
| 202043_s_at | NM_004595 | SMS | 6611 | 8.591 | 8.49 | 8.42 | 8.42 | 8.33 | 8.396 | −0.119 |
| 202055_at | AA652173 | KPNA1 | 3836 | 6.996 | 6.94 | 7 | 6.79 | 7.14 | 6.931 | −0.07 |
| 202056_at | AW051311 | KPNA1 | 3836 | 6.602 | 6.74 | 6.63 | 6.68 | 6.97 | 6.834 | −0.017 |
| 202057_at | BC002374 | KPNA1 | 3836 | 5.398 | 5.11 | 5.28 | 5.35 | 5.48 | 4.928 | 0.0619 |
| 202058_s_at | BC002374 | KPNA1 | 3836 | 7.044 | 7.18 | 7.25 | 7.12 | 7.2 | 7.139 | 0.073 |
| 202059_s_at | NM_002264 | KPNA1 | 3836 | 8.006 | 7.76 | 7.77 | 7.96 | 8.04 | 7.798 | −0.02 |
| 202067_s_at | AI861942 | LDLR | 3949 | 6.66 | 6.53 | 6.82 | 6.66 | 6.6 | 6.613 | 0.1444 |
| 202068_s_at | NM_000527 | LDLR | 3949 | 8.466 | 8.41 | 8.51 | 8.55 | 8.36 | 8.847 | 0.0933 |
| 202104_s_at | NM_003319 | SPG7 | 6687 | 6.041 | 6.13 | 6.27 | 5.97 | 6 | 5.838 | 0.0356 |
| 202106_at | NM_005895 | GOLGA3 | 2802 | 6.35 | 6.36 | 6.38 | 6.06 | 6.43 | 6.419 | −0.134 |
| 202151_s_at | NM_016172 | UBAC1 | 10422 | 6.87 | 6.82 | 6.74 | 6.74 | 6.84 | 6.885 | −0.105 |
| 202161_at | NM_002741 | PKN1 | 5585 | 3.899 | 3.98 | 4.4 | 3.9 | 4.63 | 4.589 | 0.2105 |
| 202181_at | NM_014734 | KIAA0247 | 9766 | 6.204 | 6.25 | 6.35 | 6.14 | 6.3 | 6.188 | 0.0151 |
| 202258_s_at | U50532 | N4BP2L2 | 10443 | 9.768 | 9.74 | 9.79 | 9.82 | 9.91 | 9.903 | 0.0476 |
| 202259_s_at | NM_014887 | N4BP2L2 | 10443 | 6.363 | 6.57 | 6.33 | 6.27 | 6.19 | 6.355 | −0.164 |
| 202273_at | NM_002609 | PDGFRB | 5159 | 3.586 | 3.45 | 3.29 | 3.36 | 3.34 | 3.366 | −0.196 |
| 202301_s_at | BE396879 | RSRC2 | 65117 | 8.423 | 8.5 | 8.41 | 8.53 | 8.57 | 8.532 | 0.0081 |
| 202302_s_at | NM_023032 | RSRC2 | 65117 | 8.973 | 9.02 | 9.11 | 8.93 | 9.16 | 9.196 | 0.0235 |
| 202333_s_at | AA877765 | UBE2B | 7320 | 9.592 | 9.68 | 9.66 | 9.59 | 9.6 | 9.502 | −0.014 |
| 202334_s_at | AI768723 | UBE2B | 7320 | 6.961 | 7.05 | 7.02 | 6.88 | 6.94 | 6.935 | −0.056 |
| 202335_s_at | NM_003337 | UBE2B | 7320 | 2.331 | 2.4 | 2.59 | 2.23 | 2.55 | 2.346 | 0.0488 |
| 202350_s_at | NM_002380 | MATN2 | 4147 | 3.918 | 3.78 | 3.88 | 3.94 | 4.1 | 4.22 | 0.0603 |
| 202354_s_at | AW190445 | GTF2F1 | 2962 | 6.694 | 6.82 | 6.92 | 6.85 | 7.13 | 7.175 | 0.1281 |
| 202355_s_at | BC000120 | GTF2F1 | 2962 | 7.161 | 7.15 | 7.09 | 7.1 | 7.24 | 7.271 | −0.06 |
| 202356_s_at | NM_002096 | GTF2F1 | 2962 | 6.164 | 6.08 | 5.99 | 6.22 | 6.25 | 6.103 | −0.019 |
| 202363_at | AF231124 | SPOCK1 | 6695 | 5.042 | 5.1 | 5.25 | 5.11 | 5.49 | 5.329 | 0.1084 |
| 202367_at | NM_001913 | CUX1 | 1523 | 5.365 | 5.58 | 5.34 | 5.45 | 5.39 | 5.388 | −0.075 |
| 202393_s_at | NM_005655 | KLF10 | 7071 | 8.262 | 8.33 | 8.39 | 8.19 | 8.43 | 8.222 | −0.007 |
| 202397_at | NM_005796 | NUTF2 | 10204 | 7.062 | 7.16 | 7.33 | 7.22 | 7.35 | 7.39 | 0.1584 |
| 202402_s_at | NM_001751 | CARS | 833 | 8.139 | 8.14 | 8.19 | 8.2 | 8.01 | 8.172 | 0.0542 |
| 202405_at | BF432332 | TIAL1 | 7073 | 5.17 | 5.14 | 5.23 | 5.06 | 5.36 | 5.049 | −0.009 |
| 202406_s_at | NM_003252 | TIAL1 | 7073 | 9.139 | 9.05 | 9.06 | 9.14 | 9.11 | 9.13 | 0.0078 |
| 202415_s_at | NM_012267 | HSPBP1 | 23640 | 5.97 | 5.98 | 6.1 | 6.02 | 6.03 | 6.239 | 0.0877 |
| 202424_at | NM_030662 | MAPZK2 | 5605 | 7.181 | 7.26 | 7.21 | 7.18 | 7.3 | 7.172 | −0.023 |
| 202426_s_at | BE675800 | RXRA | 6256 | 3.754 | 3.65 | 3.47 | 3.72 | 3.86 | 3.925 | −0.105 |
| 202438_x_at | BF346014 | IDS | 3423 | 4.112 | 3.62 | 4.04 | 3.55 | 4.15 | 3.849 | −0.075 |
| 202439_s_at | NM_000202 | IDS | 3423 | 6.736 | 6.59 | 6.5 | 6.65 | 6.82 | 6.952 | −0.087 |
| 202449_s_at | NM_002957 | RXRA | 6256 | 6.062 | 6.02 | 5.92 | 6.06 | 6.26 | 6.197 | −0.051 |
| 202555_s_at | NM_005965 | MYLK | 4638 | 6.427 | 6.49 | 6.52 | 6.39 | 6.39 | 6.433 | −0.009 |
| 202575_at | NM_001878 | CRABP2 | 1382 | 5.902 | 5.75 | 5.99 | 6.05 | 5.61 | 5.945 | 0.1913 |
| 202579_x_at | NM_006353 | HMGN4 | 10473 | 10.07 | 10 | 10.1 | 10.1 | 10.1 | 9.988 | 0.0471 |
| 202586_at | AA772747 | POLR2L | 5441 | 3.398 | 3.19 | 3.43 | 3.3 | 3.58 | 3.268 | 0.0661 |
| 202598_at | NM_005979 | S100A13 | 6284 | 9.267 | 9.48 | 9.33 | 9.36 | 9.27 | 9.491 | −0.029 |
| 202605_at | NM_000181 | GUSB | 2990 | 9.543 | 9.57 | 9.55 | 9.54 | 9.7 | 9.659 | −0.017 |
| 202615_at | BF222895 | GNAQ | 2776 | 8.095 | 8 | 8.17 | 8.08 | 8.15 | 8.088 | 0.075 |
| 202639_s_at | AI689052 | RANBP3 | 8498 | 5.374 | 5.43 | 5.33 | 5.43 | 5.39 | 5.644 | −0.021 |
| 202640_s_at | NM_003624 | RANBP3 | 8498 | 5.461 | 5.38 | 5.49 | 5.51 | 5.44 | 5.529 | 0.0783 |
| 202671_s_at | NM_003681 | PDXK | 8566 | 7.837 | 7.89 | 7.79 | 7.88 | 8 | 7.999 | −0.027 |
| 202672_s_at | NM_001674 | AAATF3 | 467 | 7.188 | 7.05 | 7.02 | 7.06 | 7.21 | 7.092 | −0.081 |
| 202716_at | NM_002827 | PTPN1 | 5770 | 6.621 | 6.43 | 7.05 | 6.83 | 6.75 | 6.708 | 0.4152 |
| 202733_at | NM_004199 | P4HA2 | 8974 | 9.349 | 9.4 | 9.39 | 9.26 | 9.38 | 9.367 | −0.051 |
| 202736_s_at | AA112507 | LSM4 | 25804 | 9.282 | 9.31 | 9.28 | 9.23 | 9.25 | 9.275 | −0.044 |
| 202737_s_at | NM_012321 | LSM4 | 25804 | 8.766 | 8.72 | 8.76 | 8.91 | 8.7 | 8.836 | 0.0951 |
| 202740_at | NM_000666 | ACY1 | 95 | 6.364 | 6.37 | 6.45 | 6.03 | 6.55 | 6.639 | −0.124 |
| 207255_s_at | AI354854 | GPC1 | 2817 | 3.655 | 3.5 | 3.72 | 3.66 | 3.53 | 318 | 0.109 |
| 202756_s_at | NM_002081 | GPC1 | 2817 | 5.893 | 6.21 | 5.93 | 5.92 | 5.75 | 6.065 | −0.132 |
| 202759_s_at | BE879367 | AKAP2 /// PALM2 /// | 11217 /// 114299 /// | 6.189 | 6.05 | 6.17 | 6.07 | 6.19 | 6.293 | −8E−04 |
| | | PALM2-AKAP2 | 445815 | | | | | | | |
| 202760_s_at | NM_007203 | PALM2-AKAP2 | 445815 | 6.999 | 6.79 | 7.1 | 7.02 | 7.29 | 7.317 | 0.17 |
| 202761_s_at | NM_015180 | SYNE2 | 23224 | 5.85 | 5.71 | 5.64 | 5.66 | 5.79 | 5.83 | −0.131 |
| 202797_at | NM_014016 | SACM1L | 22908 | 9.075 | 9.07 | 9 | 9.03 | 8.66 | 8.912 | −0.062 |
| 202806_at | NM_004395 | DBN1 | 1627 | 6.719 | 6.77 | 6.64 | 6.71 | 6.95 | 6.794 | −0.07 |
| 202833_s_at | NM_000295 | SEPINA1 | 5265 | 8.889 | 9.01 | 9.03 | 8.96 | 8.88 | 8.834 | 0.0448 |
| 202865_at | AI695173 | DNAJB12 | 54788 | 3.585 | 3.55 | 4.09 | 3.95 | 3.8 | 3.448 | 0.4501 |
| 202866_at | BG283782 | DNAJB12 | 54788 | 7.149 | 7.12 | 7.08 | 7.02 | 7.04 | 7.192 | −0.083 |
| 202867_s_at | NM_017626 | DNAJB12 | 54788 | 6.38 | 6.53 | 6.49 | 6.44 | 6.45 | 6.344 | 0.0121 |
| 202905_x_at | AI796269 | NBN | 4683 | 8.827 | 8.73 | 8.72 | 8.72 | 8.67 | 8.8 | −0.06 |
| 202906_s_at | AP049895 | NBN | 4683 | 7.854 | 8.02 | 7.73 | 7.87 | 8.05 | 8.119 | −0.133 |
| 202907_s_at | NM_002485 | NBN | 4683 | 7.168 | 7.07 | 7.1 | 7.11 | 7.11 | 7.074 | −0.009 |
| 202918_s_at | AF151853 | MOBKL3 | 25843 | 8.699 | 8.83 | 8.53 | 8.72 | 8.77 | 8.711 | −0.14 |
| 202919_at | NM_015387 | MOBKL3 | 25843 | 6.956 | 6.93 | 6.89 | 6.81 | 7.09 | 6.874 | −0.092 |
| 202934_at | AI761561 | HK2 | 3099 | 6.758 | 6.69 | 6.61 | 6.72 | 6.96 | 6.655 | −0.062 |
| 202950_at | NM_001889 | CRYZ | 1429 | 6.507 | 6.7 | 6.73 | 6.61 | 6.37 | 6.468 | 0.0665 |
| 202996_at | NM_021173 | POLD4 | 57804 | 6.643 | 6.59 | 6.42 | 6.62 | 6.65 | 6.742 | −0.098 |
| 203020_at | NM_014857 | RABGAP1L | 9910 | 6.659 | 6.61 | 6.7 | 6.67 | 6.84 | 6.689 | 0.051 |
| 203038_at | NM_002844 | PTPRK | 5796 | 8.357 | 8.23 | 8.43 | 8.29 | 8.64 | 8.519 | 0.0687 |
| 203051_at | NM_014952 | BAHD1 | 22893 | 3.785 | 3.76 | 4.01 | 3.75 | 3.77 | 3.795 | 0.107 |
| 203064_s_at | NM_004514 | FOXK2 | 3607 | 6.181 | 6.07 | 6.13 | 6.3 | 6.31 | 6.474 | 0.0904 |
| 203081_at | NM_020248 | CTNNBIP1 | 56998 | 5.036 | 5.46 | 5.26 | 5.32 | 5.54 | 5.144 | 0.0431 |
| 203082_at | NM_014753 | BMS1 | 9790 | 6.639 | 6.62 | 6.61 | 6.65 | 6.68 | 6.688 | −3E−04 |
| 203107_x_at | NM_002952 | RPS2 | 6187 | 13.11 | 13 | 13 | 13.1 | 13 | 13.05 | −0.028 |
| 203113_s_at | NM_001960 | EEF1D | 1936 | 10.07 | 10.2 | 9.96 | 10.1 | 9.8 | 9.925 | −0.076 |
| 203173_s_at | AW080196 | C16orf62 | 57020 | 5.871 | 5.91 | 6 | 6.06 | 5.93 | 5.684 | 0.1379 |
| 203179_at | NM_000155 | GALT | 2592 | 5.376 | 5.42 | 5.18 | 5.48 | 5.69 | 5.551 | −0.069 |
| 203188_at | NM_006876 | B3GNT1 | 11041 | 7.039 | 7.06 | 6.89 | 6.97 | 7.22 | 7.143 | −0.119 |
| 203193_at | NM_004451 | ESRRA | 2101 | 4.138 | 4.12 | 3.96 | 4.21 | 4.09 | 3.969 | −0.05 |
| 203231_s_at | AW235612 | ATXN1 | 6310 | 3.907 | 3.77 | 4.04 | 3.84 | 3.7 | 3.835 | 0.0973 |
| 203232_s_at | NM_000332 | ATXN1 | 6310 | 5.937 | 5.91 | 5.96 | 5.74 | 5.52 | 5.924 | −0.077 |
| 203234_at | NM_003364 | UPP1 | 7378 | 10.77 | 10.7 | 10.7 | 10.9 | 10.7 | 10.73 | 0.0558 |
| 203258_at | NM_006442 | DRAP1 | 10589 | 7.709 | 7.83 | 7.79 | 7.86 | 7.71 | 7.98 | 0.057 |
| 203297_s_at | BG029530 | JARID2 | 3720 | 7.323 | 7.28 | 7.49 | 7.31 | 7.22 | 7.102 | 0.0957 |
| 203298_s_at | NM_004973 | JARID2 | 3720 | 8.91 | 8.39 | 8.6 | 8.6 | 8.56 | 8.442 | 0.2085 |
| 203321_s_at | AK022588 | ADNP2 | 22850 | 7.17 | 7.3 | 7.29 | 7.2 | 7.16 | 7.475 | 0.0059 |
| 203322_at | AU145934 | ADNP2 | 22850 | 6.371 | 6.28 | 6.64 | 6.32 | 6.61 | 6.445 | 0.1555 |
| 203323_at | BF197655 | CAV2 | 858 | 9.114 | 9.08 | 9.06 | 9.11 | 9.44 | 9.423 | −0.014 |
| 203324_s_at | NM_001233 | CAV2 | 858 | 10.27 | 10.3 | 10.2 | 10.3 | 10.1 | 10.37 | −0.039 |
| 203334_at | NM_004941 | DHX8 | 1659 | 6.225 | 6.44 | 6.3 | 6.52 | 6.28 | 6.495 | 0.0768 |
| 203366_at | NM_002693 | POLG | 5428 | 7.014 | 7.05 | 7.09 | 7.02 | 7.18 | 7.2 | 0.0233 |
| 203368_at | NM_015513 | CRELD1 | 7898 | 4.852 | 4.66 | 4.36 | 4.49 | 4.75 | 4.542 | −0.33 |
| 203406_at | NM_005926 | MFAF1 | 4236 | 8.857 | 8.74 | 8.89 | 8.74 | 8.74 | 8.74 | 0.0176 |
| 203456_at | NM_007213 | PRAF2 | 11230 | 7.594 | 7.58 | 7.69 | 7.62 | 7.52 | 7.695 | 0.0664 |
| 203458_at | AI951454 | SPR | 6697 | 5.622 | 5.9 | 5.8 | 5.63 | 5.92 | 5.642 | −0.041 |
| 203499_at | NM_004431 | EPHA2 | 1969 | 7.474 | 7.41 | 7.58 | 7.47 | 7.49 | 7.391 | 0.828 |
| 203511_s_at | AF041432 | TRAPPC3 | 27095 | 8.508 | 8.63 | 8.46 | 8.38 | 8.45 | 8.371 | −0.148 |
| 203512_at | NM_014408 | TRAPPC3 | 27095 | 7.675 | 7.65 | 7.63 | 7.58 | 7.51 | 7.663 | −0.052 |
| 203515_s_at | NM_006556 | PMVK | 10654 | 6.426 | 6.27 | 6.29 | 6.14 | 6.14 | 6.437 | −0.136 |
| 203557_s_at | NM_000281 | PCBD1 | 5092 | 5.744 | 5.55 | 5.73 | 5.62 | 6.13 | 6.023 | 0.0312 |
| 203561_at | NM_021642 | FCGR2A | 2212 | 2.777 | 2.55 | 2.83 | 2.8 | 2.73 | 2.745 | 0.1479 |
| 203571_s_at | NM_006829 | C10orf116 | 10974 | 10.73 | 10.7 | 10.7 | 10.8 | 10.6 | 10.64 | 0.0049 |
| 203627_at | AI830598 | IGF3R | 3480 | 5.487 | 5.47 | 5.55 | 5.22 | 5.46 | 5.19 | −0.093 |
| 203628_at | H05812 | IGF1R | 3480 | 5.296 | 5.53 | 5.56 | 5.27 | 5.68 | 5.412 | 0.005 |
| 203710_at | NM_002222 | ITPR1 | 3708 | 6.09 | 5.81 | 6.03 | 6.06 | 6.02 | 5.692 | 0.0959 |
| 203778_at | NM_005908 | MANBA | 4126 | 5.942 | 5.7 | 5.82 | 5.7 | 5.89 | 5.866 | −0.06 |
| 203792_x_at | BC004558 | PCGF2 | 7703 | 4.027 | 4.1 | 4.36 | 4.33 | 4.06 | 4.102 | 0.1808 |
| 203793_x_at | NM_007144 | PCGF2 | 7703 | 4.527 | 4.59 | 4.3 | 4.26 | 4.22 | 4.35 | −0.281 |
| 203810_at | BG252490 | DNA3B4 | 11080 | 5.703 | 5.89 | 6.04 | 5.87 | 5.73 | 5.751 | 0.1596 |
| 203811_s_at | NM_007034 | DNAJB4 | 11080 | 6.067 | 6.19 | 6.22 | 6.3 | 6.18 | 6.017 | 0.0301 |
| 203818_s_at | NM_006802 | SF3A3 | 10946 | 7.934 | 7.74 | 7.88 | 8.03 | 8.03 | 7.976 | 0.109 |
| 203830_at | NM_022344 | C17orf75 | 64149 | 6.545 | 6.58 | 6.56 | 6.77 | 6.45 | 6.624 | 0.1055 |
| 203860_at | NM_000282 | PCCA | 5095 | 6.45 | 6.36 | 6.42 | 6.4 | 6.25 | 6.407 | 0.0039 |
| 203876_s_at | AI761713 | MMP11 | 4320 | 3.03 | 3.03 | 2.91 | 3.06 | 3.04 | 3.06 | −0.045 |
| 203877_at | NM_005940 | MMP11 | 4320 | 2.915 | 2.75 | 2.61 | 2.68 | 2.78 | 2.747 | −0.185 |
| 203878_s_at | NM_005940 | MMP11 | 4320 | 3.406 | 3.56 | 3.67 | 3.35 | 3.4 | 3.477 | 0.0305 |
| 203886_s_at | NM_001998 | FBLN2 | 2199 | 2.998 | 2.91 | 3.14 | 3.21 | 2.95 | 3.022 | 0.2189 |
| 203905_at | NM_002582 | PARN | 5073 | 7.323 | 7.38 | 7.53 | 7.6 | 7.38 | 7.271 | 0.2149 |
| 203963_at | NM_001218 | CA12 | 771 | 7.558 | 7.72 | 7.69 | 7.57 | 7.45 | 7.468 | −0.011 |
| 203966_s_at | NM_021003 | PPM1A | 5494 | 7.699 | 7.68 | 7.72 | 7.66 | 7.84 | 7.828 | 0.0022 |
| 203969_at | AU157140 | PEX3 | 8504 | 3.117 | 2.95 | 3.23 | 3.07 | 3.03 | 3.089 | 0.1136 |
| 203970_s_at | NM_003630 | PEX3 | 8504 | 7.34 | 7.23 | 7.28 | 7.21 | 7.38 | 7.185 | −0.035 |
| 203972_s_at | AB035307 | PEX3 | 8504 | 7.72 | 7.82 | 7.81 | 7.79 | 7.74 | 7.79 | 0.0325 |
| 204023_at | NM_002916 | RFC4 | 5984 | 10.75 | 10.8 | 10.8 | 10.8 | 10.7 | 10.88 | −0.017 |
| 204030_s_at | NM_014575 | SCHIP1 | 29970 | 7.243 | 7.07 | 7.25 | 7.28 | 7.08 | 7.112 | 0.1124 |
| 204053_x_at | U96180 | PTEN | 5728 | 8.803 | 8.75 | 8.81 | 8.74 | 8.78 | 8.753 | −0.002 |
| 204054_at | NM_000314 | PTEN | 5728 | 3.981 | 3.78 | 3.63 | 3.82 | 4.02 | 3.727 | −0.159 |
| 204065_at | NM_004854 | CHST10 | 9486 | 3.592 | 3.56 | 3.64 | 3.55 | 3.66 | 3.692 | −0.0229 |
| 204068_at | NM_006281 | STK3 | 6788 | 8.615 | 8.61 | 8.66 | 8.6 | 8.43 | 8.734 | 0.0137 |
| 204095_s_at | AL521391 | ELL | 8178 | 2.855 | 3.27 | 3.17 | 3.37 | 3.69 | 3.479 | 0.2055 |
| 204096_s_at | AL136771 | ELL | 8178 | 2.634 | 2.9 | 2.81 | 2.66 | 2.95 | 2.986 | −0.036 |
| 204163_at | NM_007046 | EMILIN1 | 11117 | 2.677 | 2.77 | 2.68 | 2.64 | 2.9 | 2.717 | −0.06 |
| 204170_s_at | NM_001827 | CKS2 | 1164 | 9.284 | 9.42 | 9.28 | 9.29 | 9.15 | 9.123 | −0.065 |
| 204173_at | NM_002475 | MYL6B | 140465 | 8.741 | 8.67 | 8.68 | 8.65 | 8.75 | 8.656 | −0.04 |
| 204190_at | NM_005800 | USPL1 | 10208 | 7.296 | 7.17 | 7.24 | 7.06 | 7.4 | 7.3 | −0.083 |
| 204202_at | NM_017604 | IQCE | 23288 | 4.329 | 4.32 | 4.16 | 4.26 | 4.57 | 4.737 | −0.113 |
| 204238_s_at | NM_006443 | C6orf108 | 10591 | 6.751 | 6.8 | 6.73 | 6.66 | 6.88 | 6.825 | −0.077 |
| 204292_x_at | NM_000455 | STK11 | 6794 | 3.369 | 3.4 | 3.36 | 3.59 | 3.68 | 3.58 | 0.095 |
| 204306_s_at | NM_004357 | CD151 | 977 | 7.472 | 7.58 | 7.51 | 7.56 | 7.37 | 7.533 | 0.0071 |
| 204402_at | NM_012265 | RHBDD3 | 25807 | 3.359 | 3.7 | 3.6 | 3.8 | 3.75 | 3.576 | 0.1737 |
| 204441_s_at | NM_002689 | POLA2 | 23649 | 5.942 | 5.91 | 5.89 | 6.09 | 6.11 | 5.937 | 0.0643 |
| 204442_x_at | NM_003573 | LTBP4 | 8425 | 3.957 | 4.29 | 4.29 | 4.1 | 4.13 | 4.334 | 0.0676 |
| 204503_at | NM_001988 | EVPL | 2125 | 3.103 | 3.09 | 3.17 | 3.25 | 2.97 | 3.148 | 0.11 |
| 204508_s_at | BC001012 | CA12 | 771 | 5.124 | 4.61 | 4.54 | 4.62 | 4.59 | 4.612 | −0.289 |
| 204509_at | NM_017689 | CA12 | 771 | 3.098 | 3.44 | 3.25 | 3.28 | 3.19 | 3.087 | −0.007 |
| 204537_s_at | NM_004961 | GABRE | 2564 | 3.205 | 3.31 | 3.28 | 3.39 | 3.5 | 3.172 | −0.016 |
| 204539_s_at | NM_014246 | CELSR1 | 9620 | 2.84 | 2.85 | 3.01 | 2.99 | 2.95 | 2.775 | 0.1576 |
| 204625_s_at | BF115658 | ITGB3 | 3690 | 2.9S | 3 | 3.26 | 2.84 | 3.23 | 3.198 | 0.0769 |
| 204626_s_at | J02703 | ITGB3 | 3690 | 3.266 | 3.57 | 3 | 3.15 | 3.17 | 3.16 | −0.344 |
| 204627_s_at | M35999 | ITGB3 | 3690 | 2.665 | 2.8 | 2.65 | 2.52 | 2.78 | 2.837 | −0.15 |
| 204628_s_at | NM_000212 | ITGB3 | 3690 | 3.33 | 3.27 | 3.29 | 2.97 | 3.29 | 3.302 | −0.168 |
| 204691_x_at | NM_003560 | PLA2G6 | 8398 | 3.728 | 3.59 | 3.36 | 3.69 | 3.39 | 3.135 | −0.13 |
| 204762_s_at | BE670563 | GNAO1 | 2775 | 2.954 | 2.94 | 3.05 | 2.82 | 2.64 | 2.834 | −0.019 |
| 204763_s_at | NM_020988 | GNAO1 | 2775 | 3.349 | 3.16 | 3.15 | 3.68 | 2.9 | 3.305 | 0.1574 |
| 204773_at | NM_004512 | IL11RA | 3590 | 4.762 | 4.62 | 5.22 | 4.67 | 5.29 | 5.245 | 0.2559 |
| 204785_x_at | NM_000874 | IFNAR2 | 3455 | 6.968 | 7.06 | 7.11 | 7.19 | 6.81 | 7.027 | 0.3393 |
| 204786_s_at | L41944 | IFNAR2 | 3455 | 4.907 | 4.95 | 5.03 | 4.97 | 5.19 | 5.06 | 0.07 |
| 204802_at | NM_004165 | RRAD | 6236 | 3.562 | 3.6 | 3.91 | 3.42 | 3.98 | 3.846 | 0.0793 |
| 204803_s_at | NM_004165 | RRAD | 6236 | 5.068 | 5.25 | 5.2 | 5.52 | 5.41 | 5.647 | 0.1996 |
| 204857_at | NM_003550 | MAD1L1 | 8379 | 5.045 | 5.22 | 5.08 | 5.26 | 5.3 | 5.123 | 0.0381 |
| 204883_s_at | AI968626 | HUS1 | 3364 | 7.257 | 7.3 | 7.18 | 7.22 | 7.36 | 7.511 | −0.081 |
| 204884_s_at | NM_004507 | HUS1 | 3364 | 2.827 | 2.91 | 3 | 2.98 | 2.86 | 3.003 | 0.1215 |
| 204945_at | NM_002846 | PTPRN | 5798 | 2.792 | 2.79 | 3.06 | 3.04 | 2.91 | 2.874 | 0.258 |
| 204962_s_at | NM_001809 | CENPA | 1058 | 4.843 | 4.71 | 4.77 | 5.05 | 5.19 | 4.828 | 0.1374 |
| 204981_at | NM_002555 | SLC22A18 | 5002 | 6.66 | 6.72 | 6.66 | 6.6 | 6.91 | 6.815 | −0.058 |
| 204995_at | AL567411 | CDK5R1 | 8851 | 3.928 | 3.56 | 3.99 | 3.66 | 4.33 | 3.477 | 0.0806 |
| 204996_s_at | NM_003885 | CDK5R1 | 8851 | 2.838 | 2.84 | 2.86 | 2.74 | 2.74 | 2.601 | −0.039 |
| 205003_at | NM_014705 | DOCK4 | 9732 | 6.294 | 6.11 | 6.11 | 6.12 | 6.11 | 6.066 | −0.087 |
| 205005_s_at | AW293531 | NMT2 | 9397 | 4.71 | 4.84 | 4.99 | 5.11 | 5 | 4.845 | 0.2742 |
| 205006_s_at | NM_004808 | NMT2 | 9397 | 4.834 | 4.85 | 4.92 | 4.78 | 4.83 | 4.842 | 0.0104 |
| 205048_s_at | NM_003832 | PSPH | 5723 | 5.02 | 5.19 | 4.94 | 4.86 | 4.51 | 4.769 | −0.204 |
| 205089_at | NM_003416 | ZNF7 | 7553 | 7.149 | 7.09 | 6.9 | 6.89 | 7.08 | 7.327 | −0.226 |
| 205092_x_at | NM_014950 | ZBTB1 | 22890 | 4.055 | 3.8 | 3.88 | 3.71 | 4.03 | 4.118 | −0.133 |
| 205093_at | NM_014935 | PLEKHA6 | 22874 | 3.377 | 3.08 | 3.1 | 3.43 | 3.09 | 3.133 | 0.0329 |
| 205133_s_at | NM_002157 | HSPE1 | 3336 | 8.834 | 8.89 | 8.72 | 8.88 | 8.62 | 8.625 | −0.06 |
| 205141_at | NM_001145 | ANG | 283 | 3.8 | 3.96 | 3.66 | 3.64 | 3.57 | 3.961 | −0.23 |
| 205158_at | NM_002937 | RNASE4 | 6038 | 3.614 | 3.31 | 3.63 | 3.51 | 3.4 | 3.394 | 0.1055 |
| 205163_at | NM_013292 | MYLPF | 29895 | 3.151 | 2.94 | 2.89 | 3.17 | 3.22 | 3.23 | −0.019 |
| 205175_at | NM_000221 | KHK | 3795 | 2.831 | 2.87 | 2.79 | 3.06 | 3.3 | 2.909 | 0.0737 |
| 205176_s_at | NM_014288 | ITGB3BP | 23421 | 9.879 | 9.95 | 9.88 | 9.95 | 10.1 | 9.96 | 0.0049 |
| 205189_s_at | NM_000136 | FANCC | 2176 | 4.043 | 3.85 | 4.19 | 3.8 | 3.85 | 4.077 | 0.0513 |
| 205194_at | NM_004577 | PSPH | 5723 | 7.506 | 7.49 | 7.28 | 7.56 | 7.17 | 7.341 | −0.076 |
| 205227_at | NM_002182 | IL1RAP | 3556 | 6.489 | 6.52 | 6.41 | 6.55 | 6.41 | 6.039 | −0.028 |
| 205263_at | AF082283 | BCL10 | 8915 | 8.527 | 8.56 | 8.54 | 8.42 | 8.17 | 8.278 | −0.069 |
| 205274_at | U87964 | GTPBP1 | 9567 | 3.361 | 3.04 | 2.93 | 3.19 | 2.73 | 2.959 | −0.142 |
| 205275_at | BE866976 | GTPBP1 | 9567 | 3.563 | 3.49 | 3.84 | 3.43 | 3.57 | 3.434 | 0.0917 |
| 205276_s_at | NM_004286 | GTPBP1 | 9567 | 3.036 | 3.33 | 3.23 | 3.23 | 3.05 | 3.04 | 0.0459 |
| 205292_s_at | NM_002137 | HNRNPA2B1 | 3181 | 10.67 | 10.7 | 10.6 | 10.6 | 10.4 | 10.55 | −0.087 |
| 205293_x_at | AB017120 | BAIAP2 | 10458 | 3.266 | 3.36 | 3.22 | 3.27 | 3.46 | 3.139 | −0.07 |
| 205294_at | NM_017450 | BAIAP2 | 10458 | 3.226 | 3.21 | 3.61 | 3.29 | 3.31 | 3.689 | 0.2284 |
| 205320_at | NM_005883 | APC2 | 10297 | 2.854 | 2.89 | 3.11 | 3 | 3.16 | 2.916 | 0.1812 |
| 205341_at | NM_014601 | EHD2 | 30846 | 3.617 | 3.34 | 3.82 | 3.6 | 3.66 | 3.723 | 0.2303 |
| 205349_at | NM_002068 | GNA15 | 2769 | 7.998 | 8.01 | 7.96 | 7.96 | 7.99 | 8.055 | −0.047 |
| 205359_at | NM_004274 | AKAP6 | 9472 | 2.743 | 2.74 | 2.71 | 2.89 | 2.72 | 2.685 | 0.0579 |
| 205411_at | NM_006282 | STK4 | 6789 | 3.321 | 3.34 | 3.6 | 3.11 | 3.45 | 3.151 | 0.0218 |
| 205457_at | NM_024294 | C6orf106 | 64771 | 5.73 | 5.48 | 5.87 | 5.84 | 5.59 | 5.378 | 0.2555 |
| 205463_at | NM_002607 | PDGFA | 5154 | 6.968 | 6.81 | 7.02 | 7.12 | 7.59 | 7.341 | 0.1832 |
| 205485_at | NM_000540 | RYR1 | 6261 | 3.131 | 3.46 | 3.4 | 3.23 | 3.3 | 3.213 | 0.0228 |
| 205543_at | NM_014278 | HSPA4L | 22824 | 6.381 | 6.28 | 6.69 | 6.52 | 6.43 | 6.245 | 0.2747 |
| 205579_at | NM_000861 | HRH1 | 3269 | 4.661 | 4.78 | 4.89 | 4.99 | 5.15 | 4.875 | 0.2186 |
| 205580_at | D28481 | HRH1 | 3269 | 3.844 | 3.89 | 3.87 | 4.09 | 3.68 | 3.937 | 0.114 |
| 205617_at | NM_000951 | PRRG2 | 5639 | 3.295 | 3.43 | 3.23 | 3.54 | 3.59 | 3.501 | 0.0226 |
| 205640_at | NM_000694 | ALDH3B1 | 221 | 4.379 | 4.22 | 4.22 | 4.16 | 4.11 | 4.198 | −0.106 |
| 205643_s_at | NM_004576 | PPP2R2B | 5521 | 3.137 | 3.1 | 3.03 | 3.1 | 2.8 | 3.285 | −0.053 |
| 205648_at | NM_003391 | WNT2 | 7472 | 3.662 | 3.34 | 3.72 | 3.57 | 3.28 | 3.415 | 0.1408 |
| 205674_x_at | NM_001680 | FXYD2 | 486 | 3.326 | 3.19 | 2.94 | 3.43 | 3.29 | 3.171 | −0.071 |
| 205687_at | NM_019116 | UBFD1 | 56061 | 6.279 | 6.26 | 6.34 | 6.12 | 6.11 | 6.393 | −0.044 |
| 205724_at | NM_000299 | PKP1 | 5317 | 4.243 | 4.17 | 4.14 | 4.29 | 4.04 | 4.027 | 0.0103 |
| 205829_at | NM_000413 | HSD17B1 | 3292 | 6.456 | 6.58 | 6.48 | 6.74 | 6.73 | 6.672 | 0.0934 |
| 205858_at | NM_002507 | NGFR | 4804 | 3.008 | 2.98 | 2.98 | 3.16 | 2.92 | 3.073 | 0.0787 |
| 205872_x_at | NM_022359 | PDE4DIP | 9659 | 6.775 | 6.98 | 6.98 | 6.9 | 7.37 | 7.233 | 0.1151 |
| 205873_at | NM_004278 | PIGL | 9487 | 5.582 | 5.83 | 5.83 | 5.82 | 5.75 | 5.811 | −0.069 |
| 205945_at | NM_000565 | IL6R | 3570 | 4.852 | 4.94 | 4.94 | 4.99 | 5.32 | 5.392 | 0.1718 |
| 205967_at | NM_003542 | HIST1H4A /// | 121504 /// 554313 | 9.447 | 9.38 | 9.38 | 9.43 | 8.93 | 9.19 | −0.071 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 206066_s_at | NM_002876 | RAD51C | 5889 | 7.037 | 7.24 | 6.97 | 7.15 | 7.12 | 7.217 | −0.076 |
| 206105_at | NM_002025 | AFF2 | 2334 | 3.368 | 3.19 | 3.09 | 2.94 | 3.19 | 3.404 | −0.258 |
| 206212_at | NM_001869 | CPA2 | 1358 | 3.228 | 3.06 | 3.28 | 3.17 | 3.38 | 3.316 | 0.0804 |
| 206219_s_at | NM_005428 | VAV1 | 7409 | 2.832 | 3.1 | 3.05 | 3.09 | 2.95 | 3.154 | 0.1037 |
| 206236_at | NM_005282 | GPR4 | 2828 | 2.847 | 2.81 | 2.95 | 3.1 | 3.03 | 2.909 | 0.1929 |
| 206248_at | NM_005400 | PRKCE | 5581 | 3.321 | 3.27 | 3.24 | 3.18 | 3.27 | 3.097 | −0.086 |
| 206275_s_at | NM_014632 | MICAL2 | 9645 | 3.52 | 3.21 | 3.56 | 3.51 | 3.48 | 3.8 | 0.1695 |
| 206316_s_at | NM_014708 | KNTC1 | 9735 | 7.91 | 7.98 | 7.97 | 7.95 | 8 | 7.976 | 0.0181 |
| 206322_at | NM_003490 | SYN3 | 8224 | 3.186 | 3.27 | 3.16 | 3.13 | 3.02 | 3.214 | −0.081 |
| 206324_s_at | NM_014326 | DAPK2 | 23604 | 3.357 | 3.34 | 3.54 | 3.31 | 3.58 | 3.455 | 0.0766 |
| 206342_x_at | NM_006123 | IDS | 3423 | 6.897 | 6.88 | 6.93 | 6.89 | 7.01 | 6.887 | 0.0184 |
| 206357_at | NM_025136 | OPA3 | 80207 | 4.2 | 3.85 | 4.22 | 3.98 | 4.19 | 4.264 | 0.0718 |
| 206400_at | NM_002307 | LGALS7 /// LGALS7B | 3963 /// 653499 | 5.81 | 5.64 | 5.38 | 5.49 | 5.74 | 5.936 | −0.293 |
| 206410_at | NM_021969 | NR0B2 | 8431 | 3.224 | 3.27 | 3.19 | 3.19 | 3.1 | 3.167 | −0.055 |
| 206452_x_at | NM_021131 | PPP2R4 | 5524 | 4.994 | 5.06 | 4.99 | 4.74 | 5.04 | 5.17 | −0.161 |
| 206492_at | NM_002012 | FHIT | 2272 | 4.407 | 4.73 | 4.97 | 4.48 | 4.87 | 4.892 | 0.1542 |
| 206504_at | NM_000782 | CYP24A1 | 1591 | 2.959 | 2.97 | 3.19 | 3.09 | 3.07 | 2.924 | 0.1749 |
| 206571_s_at | NM_004834 | MAP4K4 | 9448 | 6.487 | 6.39 | 6.51 | 6.31 | 6.49 | 6.558 | −0.026 |
| 206577_at | NM_003381 | VIP | 7432 | 2.688 | 2.68 | 2.68 | 2.72 | 2.66 | 2.936 | 0.0188 |
| 206582_s_at | NM_005682 | GPRS6 | 9289 | 3.599 | 3.36 | 3.43 | 3.44 | 3.5 | 3.489 | −0.044 |
| 206709_x_at | NM_005309 | GPT | 2875 | 3.231 | 2.96 | 3.16 | 3.08 | 3.16 | 3.122 | 0.028 |
| 206720_at | NM_002410 | MGATS | 4249 | 2.881 | 2.66 | 2.99 | 2.97 | 3.04 | 3.04 | 0.2108 |
| 206802_at | NM_016734 | PAX5 | 5079 | 3.284 | 3.21 | 3.11 | 2.95 | 3.32 | 3.02 | −0.214 |
| 206866_at | NM_001794 | CDH4 | 1002 | 4.32 | 4.39 | 4.3 | 4.04 | 4.59 | 4.32 | −0.183 |
| 206896_s_at | NM_005145 | GNG7 | 2788 | 4.005 | 4.15 | 3.8 | 3.82 | 3.84 | 4.005 | −0.263 |
| 206901_at | NM_024323 | C19orf57 | 79173 | 3.314 | 3.58 | 3.31 | 3.65 | 3.34 | 3.479 | 0.0338 |
| 206923_at | NM_002737 | PRKCA | 5578 | 3.128 | 3.14 | 3.22 | 2.93 | 2.88 | 3.137 | −0.061 |
| 206951_at | NM_003545 | HIST1H4A /// | 121504 /// 554313 | 3.151 | 3.41 | 3.3 | 3.21 | 3.31 | 3.27 | −0.026 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 206976_s_at | NM_006644 | HSPH1 | 10808 | 9.695 | 9.68 | 9.65 | 9.6 | 9.58 | 9.577 | −0.064 |
| 207040_s_at | NM_003932 | ST13 | 6767 | 9.71 | 9.72 | 9.6 | 9.58 | 9.47 | 9.519 | −0.128 |
| 207046_at | NM_003548 | HIST1H4A /// | 121504 /// 554313 | 2.884 | 3.1 | 2.75 | 2.68 | 2.93 | 3.103 | −0.276 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 207127_s_at | NM_021644 | HNRNPH3 | 3189 | 7.743 | 7.71 | 7.62 | 7.55 | 7.35 | 7.611 | −0.142 |
| 207188_at | NM_001258 | CDK3 | 1018 | 5.934 | 5.79 | 6.01 | 6.01 | 5.79 | 5.923 | 0.1441 |
| 207225_at | NM_001088 | AANAT | 15 | 2.736 | 2.82 | 2.52 | 2.66 | 2.57 | 2.525 | −0.188 |
| 207243_x_at | NM_001743 | CALM1 /// CALM2 /// | 801 /// 805 /// 808 | 11.48 | 11.4 | 11.5 | 11.4 | 11.4 | 11.44 | 0.0136 |
| | | CALM3 | | | | | | | | |
| 207263_x_at | NM_017599 | VEZT | 55591 | 3.422 | 3.69 | 3.72 | 3.51 | 3.53 | 3.769 | 0.0605 |
| 207323_s_at | NM_002385 | MBP | 4155 | 2.827 | 2.92 | 2.94 | 2.87 | 3.13 | 2.969 | 0.0332 |
| 207342_at | NM_001297 | CNGB1 | 1258 | 2.659 | 2.7 | 2.81 | 2.73 | 2.39 | 2.699 | 0.0925 |
| 207358_x_at | NM_012090 | MACF1 | 23499 | 7.13 | 7.26 | 7.32 | 7.44 | 7.11 | 7.07 | 0.1887 |
| 207360_s_at | NM_002531 | NTSR1 | 4923 | 3.929 | 4.11 | 4.22 | 4.18 | 3.83 | 3.732 | 0.1775 |
| 207382_at | NM_003722 | TP63 | 8626 | 3.485 | 3.57 | 3.44 | 3.39 | 3.49 | 3.518 | −0.114 |
| 207425_s_at | NM_006640 | 10-Sep | 10801 | 3.357 | 3.33 | 3.53 | 3.54 | 3.53 | 3.391 | 0.1883 |
| 207434_s_at | NM_021603 | FXYD2 | 486 | 2.957 | 3 | 2.88 | 3.04 | 3.14 | 2.889 | −0.018 |
| 207442_at | NM_000759 | CSF3 | 1440 | 3.457 | 3.6 | 3.68 | 3.36 | 3.45 | 3.557 | −0.007 |
| 207453_s_at | NM_012266 | DNAJB5 | 25822 | 4.426 | 4.51 | 4.32 | 4.1 | 4.36 | 4.667 | −0.258 |
| 207518_at | NM_003647 | DGKE | 8526 | 3.43 | 3.16 | 3.22 | 3.35 | 3.01 | 3.081 | −0.01 |
| 207525_s_at | NM_005716 | GIPC1 | 10755 | 6.346 | 6.29 | 6.18 | 6.09 | 6.21 | 6.284 | −0.185 |
| 207535_s_at | NM_002502 | NFKB2 | 4791 | 5.098 | 5.16 | 5.26 | 4.99 | 5.22 | 4.945 | −0.004 |
| 207650_x_at | NM_000955 | PTGER1 | 5731 | 3.867 | 3.72 | 3.68 | 3.82 | 3.49 | 3.498 | −0.041 |
| 207661_s_at | NM_014631 | SH3PXD2A | 9644 | 3.724 | 3.33 | 3.23 | 3.58 | 3.77 | 3.4 | −0.118 |
| 207708_at | NM_021628 | ALOXE3 | 59344 | 4.78 | 5.23 | 5.39 | 5.25 | 4.98 | 4.96 | 0.3174 |
| 207711_at | NM_015377 | C20orf117 | 140710 | 4.632 | 4.4 | 4.16 | 4.22 | 4.61 | 4.462 | −0.321 |
| 207712_at | NM_001187 | BAGE | 574 | 2.792 | 2.94 | 2.9 | 2.79 | 3.04 | 2.991 | −0.021 |
| 207714_s_at | NM_004353 | SERPINH1 | 871 | 7.075 | 7.02 | 7.07 | 7.14 | 6.78 | 6.526 | 0.0542 |
| 207760_s_at | NM_006312 | NCOR2 | 9612 | 7.753 | 7.79 | 7.79 | 7.76 | 8.13 | 7.943 | 0.0064 |
| 207821_s_at | NM_005607 | PTK2 | 5747 | 4.99 | 5.38 | 5.15 | 4.92 | 5 | 5.197 | −0.154 |
| 207832_at | NM_017451 | BAIAP2 | 10458 | 3.261 | 3.1 | 3.22 | 3.32 | 3.42 | 3.419 | 0.0922 |
| 207838_x_at | NM_020524 | PBXIP1 | 57326 | 2.985 | 3.18 | 3.16 | 3.09 | 2.89 | 3.083 | 0.0434 |
| 207921_x_at | NM_013952 | PAX8 | 7849 | 2.631 | 2.89 | 2.69 | 2.71 | 2.67 | 2.644 | −0.059 |
| 207923_x_at | NM_013953 | PAX8 | 7849 | 2.481 | 2.66 | 2.67 | 2.71 | 2.73 | 2.919 | 0.1233 |
| 207924_x_at | NM_013992 | PAX8 | 7849 | 2.554 | 2.49 | 2.67 | 2.49 | 2.44 | 2.793 | 0.0585 |
| 207929_at | NM_005314 | GRPR | 2925 | 3.493 | 3.26 | 3.35 | 3.3 | 3.36 | 3.454 | −0.052 |
| 208002_s_at | NM_007274 | ACOT7 | 11332 | 9.814 | 9.83 | 9.83 | 9.86 | 9.84 | 9.969 | 0.0221 |
| 208003_s_at | NM_006599 | NFAT5 | 10725 | 6.758 | 6.59 | 6.65 | 6.74 | 6.57 | 6.608 | 0.0218 |
| 208009_s_at | NM_014448 | ARHGEF16 | 27237 | 3.899 | 3.87 | 3.48 | 3.76 | 3.47 | 3.307 | −0.27 |
| 208018_s_at | NM_002110 | HCK | 3055 | 2.835 | 3 | 2.79 | 2.81 | 2.71 | 2.773 | −0.116 |
| 208026_at | NM_003540 | HIST1H4A /// | 121504 /// 554313 | 2.635 | 2.75 | 2.54 | 2.87 | 2.74 | 2.741 | 0.0096 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 208031_s_at | NM_000635 | RFX2 | 5990 | 3.121 | 3.16 | 2.89 | 2.78 | 3.12 | 2.991 | −0.308 |
| 208046_at | NM_003538 | HIST1H4A /// | 121504 /// 554313 | 3.3 | 3.2 | 3.11 | 2.98 | 3.09 | 3.025 | −0.203 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 208076_at | NM_003539 | HIST1H4A /// | 121504 /// 554313 | 3.133 | 3.03 | 2.87 | 2.84 | 2.87 | 3.092 | −0.222 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 208102_s_at | NM_002779 | PSD | 5662 | 2.714 | 2.95 | 2.81 | 2.96 | 2.88 | 2.951 | 0.0516 |
| 208178_x_at | NM_007118 | TRIO | 7204 | 4.66 | 4.92 | 4.93 | 4.86 | 5.1 | 5.097 | 0.1059 |
| 208180_s_at | NM_003543 | HIST1H4A /// | 121504 /// 554313 | 2.801 | 2.73 | 3.02 | 3.11 | 2.92 | 2.84 | 0.3014 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 208181_at | NM_003543 | HIST1H4A /// | 121504 /// 554313 | 2.514 | 2.7 | 2.76 | 2.66 | 2.53 | 2.705 | 0.1023 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 208252_s_at | NM_004273 | CHST3 | 9469 | 3.12 | 3.25 | 2.96 | 3.11 | 2.92 | 3.049 | −0.153 |
| 208272_at | NM_007321 | RANBP3 | 8498 | 3.382 | 3.28 | 2.89 | 3.3 | 3.33 | 3.286 | −0.234 |
| 208315_x_at | NM_003300 | TRAF3 | 7187 | 3.538 | 4.19 | 3.9 | 3.9 | 3.87 | 3.681 | 0.0376 |
| 208333_at | NM_022363 | LHX5 | 64211 | 3.143 | 3 | 2.91 | 2.79 | 2.95 | 2.831 | −0.222 |
| 208336_s_at | NM_004868 | GPSN2 | 9524 | 7.624 | 7.88 | 7.54 | 7.81 | 7.76 | 7.858 | −0.073 |
| 208424_s_at | NM_020313 | CIAPIN1 | 57019 | 6.627 | 6.59 | 6.54 | 6.42 | 6.55 | 6.575 | −0.131 |
| 208441_at | NM_015883 | IGF1R | 3480 | 2.948 | 3.19 | 2.79 | 2.94 | 3.1 | 2.931 | −0.201 |
| 208580_x_at | NM_021968 | HIST1H4A /// | 121504 /// 554313 | 4.251 | 4.09 | 3.97 | 4.17 | 4.33 | 4.678 | −0.098 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 208589_at | NM_020389 | TRPC7 | 57113 | 2.491 | 2.42 | 2.57 | 2.65 | 2.44 | 2.686 | 0.1531 |
| 208611_s_at | U83867 | SPTAN1 | 6709 | 5.623 | 5.16 | 5.6 | 5.47 | 5.46 | 5.543 | 0.1486 |
| 208615_s_at | BF795101 | PTP4A2 | 8073 | 8.698 | 8.65 | 8.62 | 8.79 | 8.68 | 8.715 | 0.0293 |
| 208616_s_at | U48297 | PTP4A2 | 8073 | 10.75 | 10.7 | 10.7 | 10.8 | 10.9 | 10.82 | 0.0299 |
| 208617_s_at | AF208850 | PTP4A2 | 8073 | 9.394 | 9.34 | 9.34 | 9.43 | 9.5 | 9.439 | 0.0202 |
| 208633_s_at | W61052 | MACF1 | 23499 | 5.803 | 5.75 | 5.99 | 5.62 | 5.58 | 5.588 | 0.0227 |
| 208634_s_at | AB029290 | MACF1 | 23499 | 8.303 | 8.32 | 8.36 | 8.46 | 8.42 | 8.277 | 0.0991 |
| 208657_s_at | AF142408 | 10-Sep | 10801 | 5.217 | 5.26 | 5.29 | 5.33 | 5.31 | 5.33 | 0.0753 |
| 208666_s_at | BE866412 | ST13 | 6767 | 5.354 | 5.32 | 5.43 | 5.22 | 5.37 | 5.364 | −0.011 |
| 208667_s_at | U17714 | ST13 | 6767 | 7.701 | 7.72 | 7.81 | 7.71 | 7.83 | 7.896 | 0.046 |
| 208684_at | U24105 | COPA | 1314 | 9.34 | 9.3 | 9.31 | 9.11 | 8.95 | 9.093 | −0.11 |
| 208687_x_at | AF352832 | HSPA8 | 3312 | 10.45 | 10.6 | 10.5 | 10.5 | 9.69 | 9.902 | −0.034 |
| 208696_at | AF275798 | CCT5 | 22948 | 10.7 | 10.6 | 10.6 | 10.6 | 10.7 | 10.69 | −0.012 |
| 208713_at | BF724216 | HNRNPUL1 | 11100 | 6.828 | 6.58 | 6.77 | 6.8 | 6.79 | 6.836 | 0.0757 |
| 208730_x_at | AA535244 | RAB2A | 5862 | 5.685 | 5.67 | 5.57 | 5.56 | 5.59 | 5.755 | −0.111 |
| 208731_at | AW158062 | RAB2A | 5862 | 8.956 | 8.87 | 8.88 | 8.96 | 8.95 | 8.926 | 0.0085 |
| 208732_at | AI743756 | RAB2A | 5862 | 6.055 | 6 | 6.02 | 6.28 | 6.22 | 6.251 | 0.1188 |
| 208733_at | AW301641 | RAB2A | 5862 | 2.986 | 3.18 | 2.83 | 3.16 | 2.87 | 3.014 | −0.089 |
| 208734_x_at | M28213 | RAB2A | 5862 | 9.128 | 9.19 | 9.13 | 9.09 | 9.13 | 9.124 | −0.051 |
| 208744_x_at | BG403660 | HSPH1 | 10808 | 9.16 | 9.25 | 9.17 | 9.19 | 8.73 | 8.96 | −0.028 |
| 208756_at | U36764 | EIF3I | 8668 | 10.15 | 10.2 | 10.1 | 10.2 | 9.99 | 10.17 | −0.014 |
| 208759_at | AF240468 | NCSTN | 23385 | 6.816 | 6.75 | 6.74 | 6.65 | 6.76 | 6.632 | −0.085 |
| 208760_at | AL031714 | UBE2I | 7329 | 6.536 | 6.27 | 6.32 | 6.67 | 6.63 | 6.598 | 0.093 |
| 208778_s_at | BC000665 | TCP1 | 6950 | 10.64 | 10.7 | 10.6 | 10.6 | 10.7 | 10.69 | −0.081 |
| 208781_x_at | AF062483 | SNX3 | 8724 | 9.631 | 9.7 | 9.56 | 9.48 | 9.62 | 9.597 | −0.143 |
| 208791_at | M25915 | CLU | 1191 | 4.238 | 4.38 | 4.42 | 4.37 | 4.55 | 4.083 | 0.0846 |
| 208792_s_at | M25915 | CLU | 1191 | 4.503 | 4.63 | 4.93 | 4.63 | 4.75 | 5.093 | 0.2066 |
| 208806_at | BE379542 | CHD3 | 1107 | 3.939 | 3.37 | 3.8 | 3.74 | 3.68 | 3.864 | 0.1143 |
| 208807_s_at | U91543 | CHD3 | 1107 | 4.516 | 4.33 | 4.43 | 4.81 | 4.23 | 4.513 | 0.1988 |
| 208810_at | AF080569 | DNAJB6 | 10049 | 8.081 | 8.04 | 8.03 | 8.15 | 7.79 | 8.034 | 0.0281 |
| 208811_s_at | AF080569 | DNAJB6 | 10049 | 7.438 | 7.47 | 7.5 | 7.31 | 7.43 | 7.381 | −0.045 |
| 208813_at | BC000498 | GOT1 | 2805 | 9.798 | 9.87 | 9.78 | 9.76 | 9.8 | 9.843 | −0.068 |
| 208814_at | AA043348 | HSPA4 | 3308 | 6.378 | 6.37 | 6.37 | 6.12 | 6.46 | 6.32 | −0.134 |
| 208815_x_at | AB023420 | HSPA4 | 3308 | 9.918 | 9.86 | 9.85 | 9.93 | 9.94 | 9.923 | 0.0011 |
| 208820_at | AL037339 | PTK2 | 5747 | 7.929 | 8 | 7.96 | 8.03 | 8.06 | 8.035 | 0.0292 |
| 208837_at | BC000027 | TMED3 | 23423 | 8.095 | 8.1 | 7.99 | 7.99 | 7.96 | 8.075 | −0.106 |
| 208858_s_at | BC004998 | FAM62A | 23344 | 7.828 | 7.61 | 7.87 | 7.75 | 7.7 | 7.685 | 0.0855 |
| 208874_x_at | BC002545 | PPP2R4 | 5524 | 5.119 | 5.22 | 5.15 | 5.2 | 5.17 | 5.271 | 0.01 |
| 208888_s_at | AI499095 | NCOR2 | 9612 | 3.018 | 2.82 | 2.71 | 3.08 | 2.94 | 2.966 | −0.023 |
| 208889_s_at | AI373205 | NCOR2 | 9612 | 3.24 | 3.39 | 3.38 | 3.51 | 3.76 | 3.406 | 0.1314 |
| 208929_x_at | BC004954 | RPL13 | 6137 | 12.48 | 12.5 | 12.4 | 12.4 | 12.3 | 12.43 | −0.073 |
| 208968_s_at | BC002568 | CIAPIN1 | 57019 | 8.277 | 8.19 | 8.19 | 8.19 | 8.33 | 8.271 | −0.041 |
| 208980_s_at | M26880 | RPS27A /// UBB /// | 6233 /// 7314 /// | 12.24 | 12.3 | 12.2 | 12.2 | 12.3 | 12.2 | −0.042 |
| | | UBC | 7316 | | | | | | | |
| 208990_s_at | AF132362 | HNRNPH3 | 3189 | 9.477 | 9.47 | 9.54 | 9.45 | 9.09 | 8.89 | 0.0184 |
| 209010_s_at | AI797657 | TRIO | 7204 | 2.616 | 2.82 | 2.94 | 2.95 | 2.93 | 2.75 | 0.2272 |
| 209011_at | BF223718 | TRIO | 7204 | 4.513 | 4.8 | 4.77 | 4.47 | 4.83 | 4.717 | −0.04 |
| 209012_at | AV718192 | TRIO | 7204 | 5.781 | 5.88 | 5.84 | 5.69 | 5.86 | 5.838 | −0.065 |
| 209013_x_at | AF091395 | TRIO | 7204 | 4.947 | 5.07 | 4.96 | 4.79 | 5.05 | 5.015 | −0.131 |
| 209015_s_at | BC002446 | DNAJB6 | 10049 | 5.645 | 5.44 | 5.4 | 5.31 | 5.49 | 5.493 | −0.187 |
| 209029_at | AF193844 | COPS7A | 50813 | 7.415 | 7.43 | 7.54 | 7.39 | 7.36 | 7.287 | 0.0458 |
| 209036_s_at | BC001917 | MDH2 | 4191 | 10.87 | 10.8 | 10.8 | 10.9 | 10.7 | 10.9 | −0.023 |
| 209050_s_at | AI421559 | RALGDS | 5900 | 5.688 | 5.66 | 5.63 | 5.51 | 5.91 | 5.698 | −0.101 |
| 209051_s_at | AF295773 | RALGDS | 5900 | 3.965 | 3.63 | 3.86 | 3.94 | 3.79 | 3.631 | 0.1007 |
| 209072_at | M13577 | MBP | 4155 | 3.155 | 3.04 | 3.31 | 3.29 | 3.11 | 3.056 | 0.2043 |
| 209117_at | U79458 | WBP2 | 23558 | 6.616 | 6.23 | 6.62 | 6.71 | 6.51 | 6.524 | 0.2401 |
| 209130_at | BC003686 | SNAP23 | 8773 | 8.606 | 8.47 | 8.57 | 8.57 | 8.46 | 8.418 | 0.0319 |
| 209131_s_at | U55936 | SNAP23 | 8773 | 4.299 | 4.28 | 4.03 | 4.21 | 4.45 | 4.048 | −0.173 |
| 209179_s_at | BC003164 | MBOAT7 | 79143 | 5.462 | 5.26 | 3.52 | 5.24 | 5.59 | 5.471 | 0.0191 |
| 209214_s_at | BC004817 | EWSR1 | 2130 | 7.056 | 7.03 | 6.93 | 6.95 | 6.98 | 6.986 | −0.1 |
| 209216_at | BC000464 | WDR45 | 11152 | 7.744 | 7.72 | 7.71 | 7.94 | 7.78 | 7.848 | 0.0959 |
| 209217_s_at | BC000464 | WDR45 | 11152 | 6.973 | 6.83 | 6.84 | 6.76 | 6.88 | 6.806 | −0.103 |
| 209229_s_at | BC002799 | SAPS1 | 22870 | 4.058 | 4.02 | 4.34 | 4.09 | 4.17 | 4.211 | 0.1746 |
| 209263_x_at | BC000389 | TSPAN4 | 7106 | 7.792 | 7.87 | 7.79 | 7.93 | 7.76 | 7.754 | 0.0285 |
| 209264_s_at | AF054841 | TSPAN4 | 7106 | 6.979 | 7.06 | 6.99 | 6.87 | 7.02 | 7.205 | −0.086 |
| 209282_at | AF309082 | PRKD2 | 25865 | 3.811 | 4.02 | 3.42 | 3.95 | 3.94 | 3.674 | −0.232 |
| 209380_s_at | AF146074 | ABCCS | 10057 | 4.829 | 5 | 4.78 | 4.91 | 5.14 | 5.01 | −0.07 |
| 209388_at | BC000927 | PAPOLA | 10914 | 9.065 | 9.23 | 9.02 | 9.15 | 9.37 | 9.235 | −0.066 |
| 209428_s_at | BG420865 | ZFPL1 | 7542 | 6.322 | 6.78 | 6.59 | 6.65 | 6.43 | 6.152 | 0.0689 |
| 209453_at | M81768 | SLC9A1 | 6548 | 5.352 | 5.64 | 5.72 | 5.67 | 5.35 | 5.435 | 0.198 |
| 209493_at | AF338650 | PDZD2 | 23037 | 3.487 | 3.74 | 3.82 | 3.72 | 3.55 | 3.462 | 0.1531 |
| 209502_s_at | BC002495 | BAIAP2 | 10458 | 4.041 | 3.99 | 3.98 | 3.8 | 4.16 | 3.784 | −0.127 |
| 209516_at | U50383 | SMYD5 | 10322 | 4.321 | 4.05 | 4.52 | 4.18 | 4.55 | 4.23 | 0.1611 |
| 209552_at | BC001060 | PAX8 | 7849 | 3.077 | 3.07 | 2.9 | 3.14 | 3.05 | 2.961 | −0.056 |
| 209563_x_at | BC000454 | CALM1 /// CALM2 /// | 801 /// 808 /// 808 | 9.743 | 9.72 | 9.74 | 9.8 | 9.9 | 9.758 | 0.0405 |
| | | CALM3 | | | | | | | | |
| 209575_at | BC001903 | IL10RB | 3588 | 7.241 | 7.33 | 7.51 | 7.31 | 7.31 | 7.42 | 0.1239 |
| 209579_s_at | AL556619 | MBD4 | 8930 | 9.917 | 9.91 | 9.89 | 9.85 | 10 | 10.06 | −0.042 |
| 209580_s_at | AF114784 | MBD4 | 8930 | 7.334 | 7.23 | 7.22 | 7.15 | 7.51 | 7.266 | −0.095 |
| 209590_at | AL57414 | BMP7 | 655 | 3.248 | 3.05 | 2.77 | 2.96 | 3.06 | 2.8 | −0.284 |
| 209591_s_at | M60316 | BMP7 | 655 | 3.008 | 2.83 | 2.74 | 2.8 | 2.96 | 2.721 | −0.148 |
| 209626_s_at | AL202969 | OSBPL3 | 26031 | 5.976 | 5.94 | 5.98 | 5.97 | 6.11 | 6.044 | 0.0183 |
| 209627_s_at | AY008372 | OSBPL3 | 26031 | 4.967 | 4.7 | 5.17 | 5.16 | 5.41 | 5.275 | 0.3316 |
| 209636_at | BC002844 | NFKB2 | 4791 | 3.035 | 3.05 | 2.86 | 2.88 | 2.74 | 3.012 | −0.17 |
| 209667_at | BF033242 | CES2 | 8824 | 8.347 | 8.25 | 8.32 | 8.38 | 8.51 | 8.415 | 0.0545 |
| 209668_x_at | D50579 | CES2 | 8824 | 6.493 | 6.49 | 6.57 | 6.51 | 6.57 | 6.563 | 0.0449 |
| 209674_at | D83702 | CRY1 | 1407 | 7.313 | 7.17 | 7.25 | 7.26 | 7.34 | 7.17 | 0.0153 |
| 209675_s_at | BC004242 | HNRNPUL1 | 11100 | 5.245 | 5.04 | 4.97 | 5.34 | 5.19 | 5.378 | 0.0113 |
| 209700_x_at | AB042555 | PDE4DIP | 9659 | 3.276 | 3.37 | 3.46 | 3.31 | 3.73 | 3.558 | 0.0635 |
| 209736_at | AF116571 | SOX13 | 9580 | 5.103 | 5.21 | 5.19 | 5.25 | 5.26 | 5.25 | 0.0647 |
| 209786_at | BC001282 | HMGN4 | 10473 | 9.164 | 8.89 | 8.97 | 8.97 | 8.96 | 8.92 | −0.057 |
| 209787_s_at | BC001282 | HMGN4 | 10473 | 10.13 | 10.1 | 10.1 | 10.2 | 10.2 | 10.17 | 0.0081 |
| 209805_at | U14658 | PMS2 /// PMS2CL | 441194 /// 5395 | 6.712 | 6.83 | 6.81 | 6.71 | 6.83 | 6.967 | −0.014 |
| 209807_s_at | U18759 | NFIX | 4784 | 3.281 | 3.06 | 3.35 | 3.16 | 3.3 | 3.459 | 0.0843 |
| 209820_s_at | BC002361 | TBL3 | 10607 | 4.539 | 4.7 | 4.67 | 4.53 | 4.59 | 4.598 | −0.013 |
| 209834_at | AB017915 | CHST3 | 9469 | 4.824 | 4.13 | 4.6 | 4.2 | 4.14 | 4.123 | −0.079 |
| 209849_s_at | AF029669 | RAD51C | 5889 | 8.699 | 8.67 | 8.66 | 8.65 | 8.83 | 8.822 | −0.028 |
| 209857_s_at | AF245447 | SPHK2 | 56848 | 3.028 | 3.14 | 3.16 | 3.57 | 3.15 | 3.4 | 0.2793 |
| 209863_s_at | AF091627 | TP63 | 8626 | 6.228 | 6.14 | 6.28 | 6.12 | 6.35 | 6.292 | 0.0146 |
| 209885_at | BC001338 | RHOD | 29984 | 7.871 | 7.61 | 7.71 | 7.61 | 7.94 | 7.914 | −0.082 |
| 209899_s_at | AF217197 | PUF60 | 22827 | 7.772 | 7.72 | 7.67 | 7.67 | 7.73 | 7.707 | −0.076 |
| 209934_s_at | AF225981 | ATP2C1 | 27032 | 6.882 | 6.73 | 6.68 | 6.76 | 6.64 | 6.583 | −0.09 |
| 209935_at | AF225981 | ATP2C1 | 27032 | 7.38 | 7.37 | 7.35 | 7.39 | 7.27 | 7.401 | −0.004 |
| 210011_s_at | BC000527 | EWSR1 | 2130 | 5.986 | 6.01 | 5.79 | 5.86 | 5.83 | 5.788 | −0.175 |
| 210012_s_at | BC000527 | EWSR1 | 2130 | 3.092 | 3.47 | 3.35 | 3.26 | 3.27 | 3.46 | 0.0229 |
| 210043_at | AF334946 | FRMD8 | 83786 | 3.684 | 3.87 | 3.65 | 3.58 | 3.83 | 3.521 | −0.162 |
| 210083_at | AF071542 | SEMA7A | 8482 | 3.298 | 3.42 | 3.32 | 3.67 | 3.21 | 3.377 | 0.138 |
| 210110_x_at | AF132363 | HNRNPH3 | 3189 | 6.895 | 6.8 | 6.94 | 6.65 | 6.67 | 6.823 | −0.054 |
| 210117_at | AF311312 | SPAG1 | 6674 | 5.903 | 5.73 | 6.12 | 5.87 | 6.01 | 5.953 | 0.1811 |
| 210120_s_at | BC004349 | RANBP3 | 8498 | 4.398 | 4.48 | 4.26 | 4.13 | 4.41 | 4.406 | −0.241 |
| 210125_s_at | AF044773 | BANF1 | 8815 | 8.851 | 8.63 | 8.92 | 8.79 | 8.85 | 8.883 | 0.1163 |
| 210130_s_at | AF096304 | TM7SF2 | 7108 | 4.481 | 4.41 | 4.41 | 4.17 | 4.02 | 4.159 | −0.157 |
| 210136_at | AW070431 | MBP | 4155 | 6.082 | 6.25 | 6.32 | 6.35 | 6.41 | 6.57 | 0.1647 |
| 210150_s_at | BC003355 | LAMA5 | 3911 | 6.603 | 6.63 | 6.79 | 6.64 | 6.77 | 6.623 | 0.0972 |
| 210180_s_at | U87836 | SFRS10 | 6434 | 7.793 | 7.84 | 7.85 | 7.8 | 7.83 | 7.816 | 0.0063 |
| 210211_s_at | AF028832 | HSP90AA1 | 3320 | 10.89 | 10.8 | 10.9 | 10.9 | 10.7 | 10.72 | 0.0054 |
| 210233_at | AF167343 | IL1RAP | 3556 | 5.252 | 5.56 | 5.39 | 5.71 | 5.68 | 5.732 | 0.1452 |
| 210255_at | U84138 | RAD51L1 | 5890 | 4.039 | 4 | 4.14 | 3.78 | 4.17 | 4.067 | −0.064 |
| 210305_at | AB042557 | PDE4DIP | 9659 | 3.863 | 3.91 | 3.71 | 3.93 | 4.11 | 4.256 | −0.066 |
| 210307_s_at | AL136796 | KLHL25 | 64410 | 4.916 | 5.04 | 5.16 | 5.02 | 4.82 | 5.078 | 0.1092 |
| 210331_at | AB048365 | HECW1 | 23072 | 2.892 | 2.86 | 2.86 | 3.01 | 2.93 | 2.777 | 0.058 |
| 210338_s_st | AB034951 | HSPA8 | 3312 | 10.55 | 10.6 | 10.7 | 10.5 | 9.86 | 9.976 | −0.005 |
| 210378_s_at | BC004118 | SSNA1 | 8636 | 6.105 | 6.02 | 6.15 | 5.94 | 6.21 | 6.153 | −0.018 |
| 210407_at | AF070670 | PPM1A | 5494 | 6.828 | 6.63 | 6.79 | 6.91 | 7.1 | 7.001 | 0.1183 |
| 210426_x_at | U04897 | RORA | 6095 | 3.907 | 4.03 | 3.98 | 3.63 | 3.62 | 4.046 | −0.161 |
| 210436_at | BC005220 | CCT8 | 10694 | 3.034 | 2.9 | 2.95 | 3.17 | 3.14 | 2.844 | 0.0903 |
| 210461_s_at | BC002448 | ABLIM1 | 3983 | 6.255 | 6.1 | 6.22 | 5.98 | 6.1 | 5.944 | −0.082 |
| 210479_s_at | L14611 | RORA | 6095 | 4.11 | 4.05 | 3.83 | 4.03 | 3.75 | 3.877 | −0.148 |
| 210550_s_at | L26584 | RASGRF1 | 5923 | 3.065 | 3333 | 3.51 | 333 | 3.43 | 3.521 | 0.223 |
| 210554_s_at | BC002486 | CTBP2 | 1488 | 9.43 | 9.37 | 9.26 | 9.27 | 9.09 | 9.163 | −0.137 |
| 210574_s_at | AF241788 | NUDC | 10726 | 8.515 | 8.53 | 8.28 | 8.45 | 8.45 | 8.493 | −0.155 |
| 210575_at | AF241788 | NUDC | 10726 | 2.778 | 3.3 | 2.74 | 3.06 | 3.17 | 3.045 | −0.139 |
| 210588_x_at | L32610 | HNRNPH3 | 3189 | 8.547 | 8.58 | 8.52 | 8.42 | 8.44 | 8.431 | −0.095 |
| 210628_x_at | AF051344 | LTBP4 | 8425 | 3.749 | 3.42 | 3.59 | 3.9 | 3.95 | 3.626 | 0.1622 |
| 210647_x_at | AF102988 | PLA2G6 | 8398 | 3.411 | 3.83 | 3.93 | 3.65 | 3.87 | 3.752 | 0.1672 |
| 210648_x_at | AB047360 | SNX3 | 8724 | 11.22 | 11.2 | 11.2 | 11.1 | 11.3 | 11.25 | −0.066 |
| 210666_at | AF050145 | IDS | 3423 | 4.572 | 4.72 | 4.66 | 4.91 | 4.99 | 4.697 | 0.1395 |
| 210691_s_at | AF275803 | CACYBP | 27101 | 8.937 | 8.84 | 8.8 | 8.84 | 8.47 | 8.742 | −0.069 |
| 210735_s_at | BC000278 | CA12 | 771 | 5.299 | 5.53 | 5.57 | 5.26 | 5.14 | 4.999 | 0.0017 |
| 210752_s_at | AF213666 | MLX | 6945 | 4.278 | 4.52 | 4.39 | 4.53 | 4.66 | 4.56 | 0.0576 |
| 210769_at | U18945 | CNGB1 | 1258 | 3.318 | 3.24 | 3.06 | 3.13 | 3.69 | 3.196 | −0.181 |
| 210780_at | AB006589 | ESR2 | 2100 | 3.111 | 3.26 | 2.76 | 3.26 | 3.15 | 2.891 | −0.173 |
| 210821_x_at | BC002703 | CENPA | 1058 | 3.666 | 3.43 | 3.71 | 3.62 | 3.62 | 3.689 | 0.1122 |
| 210835_s_at | AF222711 | CTBP2 | 1488 | 8.942 | 8.89 | 8.84 | 8.91 | 8.82 | 8.811 | −0.037 |
| 210878_s_at | BC001202 | JMJD1B | 51780 | 4.72 | 4.77 | 4.46 | 4.53 | 4.68 | 4.739 | −0.248 |
| 210933_s_at | BC004908 | FSCN1 | 6624 | 6.497 | 6.24 | 6.66 | 6.59 | 6.81 | 6.578 | 0.2586 |
| 210956_at | U42387 | PPYR1 | 5540 | 2.948 | 3.12 | 2.84 | 3.11 | 2.96 | 3.002 | −0.057 |
| 210957_s_at | L76569 | AFF2 | 2334 | 2.799 | 2.88 | 2.77 | 2.75 | 2.95 | 2.868 | −0.083 |
| 210984_x_at | U95089 | EGFR | 1956 | 5.656 | 5.62 | 5.68 | 6.17 | 5.66 | 6.045 | 0.287 |
| 211004_s_at | BC002553 | ALDH3B1 | 221 | 4.745 | 5.24 | 4.87 | 4.79 | 4.54 | 5.011 | −0.163 |
| 211008_s_at | BC000744 | UBE2I | 7329 | 3.038 | 3.03 | 2.97 | 3.08 | 2.89 | 2.881 | −0.013 |
| 211015_s_at | L12723 | HSPA4 | 3308 | 9.586 | 9.59 | 9.46 | 9.56 | 9.55 | 9.522 | −0.077 |
| 211016_x_at | BC002526 | HSPA4 | 3308 | 8.027 | 8.06 | 8.01 | 7.91 | 7.85 | 7.915 | −0.086 |
| 211028_s_at | BC006233 | KHK | 3795 | 3.086 | 3.23 | 2.99 | 3.29 | 3.23 | 3.081 | −0.019 |
| 211037_s_at | BC006309 | MBOAT7 | 79143 | 4.028 | 3.8 | 3.98 | 4.09 | 3.8 | 4.045 | 0.1215 |
| 211078_s_at | Z25422 | STK3 | 6788 | 4.651 | 4.59 | 4.76 | 4.5 | 4.52 | 4.678 | 0.0102 |
| 211085_s_at | Z25430 | STK4 | 6789 | 6.873 | 6.76 | 6.52 | 7.14 | 6.62 | 6.847 | 0.013 |
| 211093_at | U31973 | PDE6C | 5146 | 2.447 | 2.45 | 2.54 | 2.64 | 2.43 | 2.436 | 0.1411 |
| 211099_s_at | U58837 | CNGB1 | 1258 | 3.059 | 2.84 | 2.97 | 2.78 | 2.77 | 2.699 | −0.071 |
| 211117_x_at | AF124790 | ESR2 | 2100 | 2.822 | 3.03 | 2.74 | 2.67 | 2.92 | 2.66 | −0.225 |
| 211118_x_at | AF051428 | ESR2 | 2100 | 3.031 | 3.01 | 3 | 2.86 | 2.92 | 2.885 | −0.09 |
| 211119_at | AF060555 | ESR2 | 2100 | 2.535 | 2.49 | 2.54 | 2.65 | 2.48 | 2.519 | 0.0856 |
| 211120_x_at | AB006590 | ESR2 | 2100 | 2.936 | 3.04 | 2.7 | 2.55 | 2.6 | 2.857 | −0.365 |
| 211137_s_at | AF189723 | ATP2C1 | 27032 | 5.03 | 5.4 | 5.29 | 5.2 | 5.28 | 5.312 | 0.0286 |
| 211194_s_at | AB010153 | TP63 | 8626 | 3.659 | 3.3 | 3.18 | 3.73 | 3.78 | 3.725 | −0.025 |
| 211195_s_at | AF116771 | TP63 | 8626 | 3.043 | 3.18 | 3.06 | 3.19 | 2.97 | 3.014 | 0.0162 |
| 211200_s_at | BC002836 | EFCAB2 | 84288 | 5.967 | 6.17 | 6.01 | 6.31 | 6.2 | 6.002 | 0.0922 |
| 211225_at | U27329 | FUT5 | 2527 | 3.697 | 3.49 | 3.57 | 3.43 | 3.36 | 3.448 | −0.092 |
| 211259_s_at | BC004248 | BMP7 | 655 | 3.02 | 2.98 | 3.01 | 3.31 | 2.94 | 3.101 | 0.1625 |
| 211260_at | BC004248 | BMP7 | 655 | 3.58 | 3.77 | 3.64 | 3.58 | 3.75 | 3.939 | −0.066 |
| 211266_s_at | U35399 | GPR4 | 2828 | 2.888 | 2.8 | 2.87 | 3.02 | 2.85 | 2.695 | 0.1051 |
| 211277_x_at | BC004369 | APP | 351 | 6.144 | 6.15 | 6.02 | 6.07 | 5.92 | 5.953 | −0.105 |
| 211296_x_at | AB009010 | RPS27A /// UBB /// | 6233 /// 7314 /// | 13.04 | 13 | 13.1 | 13 | 13.1 | 12.99 | 0.0185 |
| | | UBC | 7316 | | | | | | | |
| 211323_s_at | L38019 | ITPR1 | 3708 | 3.8 | 3.58 | 3.4 | 3.26 | 3.64 | 3.599 | −0.364 |
| 211345_x_at | AF119850 | EEF1G | 1937 | 12.37 | 12.3 | 12.3 | 12.3 | 12.1 | 12.32 | −0.065 |
| 211426_x_at | U40038 | GNAQ | 2776 | 4.067 | 4.03 | 3.92 | 4.12 | 3.71 | 3.813 | −0.032 |
| 211428_at | AF119873 | SERPINA1 | 5265 | 2.957 | 2.88 | 2.8 | 2.84 | 2.83 | 2.886 | −0.099 |
| 211429_s_at | AF119873 | SERPINA1 | 5265 | 9.559 | 9.54 | 9.53 | 9.58 | 9.38 | 9.457 | 0.0009 |
| 211439_at | AF055270 | SFRS7 | 6432 | 3.494 | 3.51 | 3.5 | 3.18 | 3.43 | 3.006 | −0.163 |
| 211524_at | U09609 | NFKB2 | 4791 | 3.179 | 2.92 | 3 | 2.9 | 2.53 | 3.04 | −0.103 |
| 211550_at | AF125253 | EGFR | 1956 | 3.118 | 2.9 | 3.15 | 3.16 | 2.81 | 2.983 | 0.144 |
| 211551_at | K03193 | EGFR | 1956 | 3.476 | 3.49 | 3.49 | 3.46 | 3.31 | 3.381 | −0.011 |
| 211579_at | U95204 | ITGB3 | 3690 | 2.892 | 2.72 | 2.91 | 2.8 | 2.76 | 2.815 | 0.0491 |
| 211607_x_at | U48722 | EGFR | 1956 | 5.564 | 5.86 | 5.59 | 5.66 | 5.52 | 5.728 | −0.089 |
| 211685_s_at | AF251061 | NCALD | 83988 | 3.097 | 3.22 | 3.19 | 3.2 | 3.25 | 3.448 | 0.0346 |
| 211711_s_at | BC005821 | PTEN | 5728 | 5.763 | 5.7 | 5.92 | 5.66 | 5.66 | 5.978 | 0.0583 |
| 211730_s_at | BC005903 | POLR2L | 5441 | 7.702 | 7.82 | 7.7 | 7.8 | 7.71 | 7.865 | −0.006 |
| 211751_at | BC005949 | PDE4DIP | 9659 | 4.579 | 4.28 | 3.86 | 4.17 | 4.34 | 4.424 | −0.411 |
| 211761_s_at | BC005975 | CACYBP | 27101 | 9.14 | 9.13 | 9.07 | 9.08 | 9.26 | 9.104 | −0.063 |
| 211763_s_at | BC005979 | UBE2B | 7320 | 6.745 | 6.54 | 6.82 | 6.79 | 6.73 | 6.831 | 0.1622 |
| 211782_at | BC006170 | IDS | 3423 | 2.816 | 2.76 | 2.78 | 2.56 | 2.61 | 2.633 | −0.119 |
| 211790_s_at | AF010404 | MLL2 | 8085 | 2.765 | 2.7 | 2.59 | 2.85 | 2.69 | 2.619 | −0.013 |
| 211828_s_at | AF172268 | TNIK | 23043 | 3.396 | 3.22 | 3.08 | 3.23 | 3.1 | 3.286 | −0.157 |
| 211834_s_at | AB042841 | TP63 | 8626 | 3.059 | 3.27 | 3.05 | 3.09 | 3.07 | 3.021 | −0.093 |
| 211907_s_at | AB044555 | PARD6B | 84612 | 2.765 | 2.61 | 2.63 | 2.84 | 2.52 | 2.9 | 0.0431 |
| 211927_x_at | BE963164 | EEF1G | 1937 | 12.64 | 12.6 | 12.6 | 12.6 | 12.5 | 12.63 | −0.036 |
| 211943_x_at | AL565449 | TPT1 | 7178 | 13.06 | 13.1 | 13 | 13 | 12.9 | 13.06 | −0.066 |
| 211968_s_at | AI962933 | HSP90AA1 | 3320 | 11.02 | 11 | 11 | 10.9 | 10.8 | 10.88 | −0.048 |
| 211969_at | BG420237 | HSP90AA1 | 3320 | 11.89 | 11.9 | 11.8 | 11.8 | 11.8 | 11.74 | −0.125 |
| 211984_at | AI653730 | CALM1 /// CALM2 /// | 801 /// 805 /// 808 | 7.424 | 7.31 | 7.37 | 7.25 | 7.7 | 7.448 | −0.056 |
| | | CALM3 | | | | | | | | |
| 211985_s_at | AI653730 | CALM1 /// CALM2 /// | 801 /// 805 /// 808 | 5.392 | 5.53 | 5.75 | 5.54 | 5.72 | 5.749 | 0.1799 |
| | | CALM3 | | | | | | | | |
| 212009_s_at | AL553320 | STIP1 | 10963 | 8.917 | 8.93 | 8.88 | 8.75 | 8.78 | 8.763 | −0.105 |
| 212012_at | BF342851 | PXDN | 7837 | 8.196 | 8.3 | 8.17 | 8.13 | 8.35 | 8.173 | −0.099 |
| 212013_at | D86983 | PXDN | 7837 | 6.671 | 6.67 | 5.62 | 6.61 | 6.68 | 6.62 | −0.059 |
| 212027_at | AI925305 | RBM25 | 58517 | 8.593 | 8.78 | 8.6 | 8.64 | 8.4 | 8.766 | −0.067 |
| 212028_at | BE466128 | RBM25 | 58517 | 8.319 | 8.39 | 8.38 | 8.28 | 8.34 | 8.247 | −0.026 |
| 212030_at | BG251218 | RBM25 | 58517 | 7.219 | 7.06 | 7.32 | 7.16 | 7.15 | 7.208 | 0.1002 |
| 212031_at | AV757384 | RBM25 | 58517 | 8.244 | 8.21 | 8.35 | 8.2 | 8.29 | 8.305 | 0.0514 |
| 212032_s_at | AL046054 | PTOV1 | 53635 | 5.503 | 5.59 | 5.57 | 5.55 | 5.68 | 5.419 | 0.0148 |
| 212033_at | BF055107 | RBM25 | 58517 | 8.403 | 8.28 | 8.3 | 8.22 | 8.38 | 8.286 | −0.08 |
| 212070_at | AL554008 | GPRS6 | 9289 | 6.306 | 6.34 | 6.36 | 6.42 | 6.6 | 6.587 | 0.0634 |
| 212076_at | AI701430 | MLL | 4297 | 6.208 | 6.2 | 6.17 | 6.01 | 6.02 | 6.126 | −0.116 |
| 212078_s_at | AA704766 | MLL | 4297 | 6.082 | 6.16 | 6.25 | 6.1 | 5.93 | 6.043 | 0.0537 |
| 212079_s_at | AA715041 | MLL | 4297 | 6.461 | 6.24 | 6.34 | 6.37 | 6.01 | 6.131 | 0.0006 |
| 212080_at | AV714029 | MLL | 4297 | 5.525 | 5.83 | 6.09 | 5.83 | 6.27 | 5.642 | 0.2789 |
| 212082_s_at | BE734356 | MYL6 /// MYL6B | 140465 /// 4637 | 10.65 | 10.8 | 10.7 | 10.6 | 10.6 | 10.59 | −0.098 |
| 212088_at | BF570122 | PMPCA | 23203 | 7.165 | 7.32 | 7.3 | 7.38 | 7.28 | 7.293 | 0.0954 |
| 212125_at | NM_002883 | RANGAP1 | 5905 | 6.101 | 6.11 | 5.9 | 5.99 | 5.89 | 6.01 | −0.158 |
| 212127_at | BE379408 | RANGAP1 | 5905 | 4.959 | 5.13 | 5.22 | 4.96 | 5.1 | 5.162 | 0.0485 |
| 212191_x_at | AW574664 | RPL13 | 6137 | 12.69 | 12.7 | 12.6 | 12.7 | 12.7 | 12.69 | −0.078 |
| 212194_s_at | AI418892 | TM9SF4 | 9777 | 6.374 | 6.25 | 6.4 | 6.34 | 6.25 | 6.283 | 0.0588 |
| 212198_s_at | AL515964 | TM9SF4 | 9777 | 4.746 | 4.83 | 4.7 | 4.87 | 4.8 | 4.625 | −9E−04 |
| 212221_x_at | AV703259 | IDS | 3423 | 7.412 | 7.39 | 7.5 | 7.42 | 7.64 | 7.546 | 0.0585 |
| 212223_at | AI926544 | IDS | 3423 | 5.584 | 5.65 | 5.83 | 5.85 | 5.89 | 5.798 | 0.2274 |
| 212228_s_at | AC004382 | COQ9 | 57017 | 7.917 | 8.11 | 7.98 | 8.07 | 8.05 | 8.05 | 0.014 |
| 212255_s_at | AK001684 | ATP2C1 | 27032 | 6.365 | 6.28 | 6.53 | 6.4 | 6.48 | 6.416 | 0.1426 |
| 212259_s_at | BF344265 | PBXIP1 | 57326 | 3.519 | 3.65 | 3.71 | 3.36 | 3.59 | 3.551 | −0.051 |
| 212284_x_at | BG498776 | TPT1 | 7178 | 13.29 | 13.3 | 13.3 | 13.2 | 13.2 | 13.24 | −0.041 |
| 212317_at | AK022910 | TNPO3 | 23534 | 7.549 | 7.39 | 7.69 | 7.55 | 7.37 | 7.531 | 0.1527 |
| 212318_at | NM_012470 | TNPO3 | 23534 | 7.466 | 7.5 | 7.66 | 7.74 | 7.39 | 7.52 | 0.2154 |
| 212338_at | AA621962 | MYO1D | 4642 | 3.611 | 3.01 | 3.42 | 3.39 | 3.57 | 3.693 | 0.0961 |
| 212348_s_at | AB011173 | AOF2 | 23028 | 6.91 | 6.84 | 6.98 | 6.89 | 6.96 | 6.893 | 0.06 |
| 212367_at | AI799061 | FEM1B | 10116 | 6.841 | 6.85 | 6.87 | 6.91 | 7.03 | 7.119 | 0.0454 |
| 212373_at | AW139179 | FEM1B | 10116 | 5.973 | 5.65 | 5.6 | 5.81 | 5.85 | 5.916 | −0.105 |
| 212374_at | NM_015322 | FEM1B | 10116 | 5.632 | 5.47 | 5.67 | 5.29 | 5.75 | 5.693 | −0.07 |
| 212394_at | D42044 | KIAA0090 | 23065 | 5.293 | 5.13 | 5.33 | 5.43 | 5.46 | 5.52 | 0.1697 |
| 212395_s_at | BF197122 | KIAA0090 | 23065 | 6.519 | 6.48 | 6.69 | 6.52 | 6.52 | 6.684 | 0.1048 |
| 212396_s_at | AI143233 | KIAA0090 | 23065 | 6.9 | 6.9 | 6.81 | 6.67 | 7.01 | 6.709 | −0.16 |
| 212411_at | BE747342 | IMP4 | 92856 | 7.82 | 7.81 | 7.73 | 7.8 | 7.95 | 7.921 | −0.051 |
| 212421_at | AB023147 | C22orf9 | 23313 | 5.336 | 5.03 | 5.29 | 5.1 | 5.25 | 5.185 | 0.0088 |
| 212422_at | AL547263 | PDCD11 | 22984 | 6.839 | 6.84 | 6.71 | 6.88 | 6.84 | 6.893 | −0.045 |
| 212424_at | AW026194 | PDCD11 | 22984 | 6.274 | 6.15 | 6.27 | 6.14 | 6.52 | 6.348 | −0.005 |
| 212433_x_at | AA630314 | RPS2 | 6187 | 12.79 | 12.8 | 12.8 | 12.8 | 12.7 | 12.81 | −0.007 |
| 212445_s_at | AI357376 | NEDD4L | 23327 | 5.231 | 5.24 | 5.05 | 4.99 | 5.28 | 5.378 | −0.221 |
| 212448_at | AB007899 | NEDD4L | 23327 | 3.743 | 3.92 | 4.08 | 3.69 | 4.23 | 4.185 | 0.0544 |
| 212458_at | H97931 | SPRED2 | 200734 | 6.088 | 5.93 | 6 | 5.8 | 5.8 | 5.996 | −0.114 |
| 212461_at | BF793951 | AZIN1 | 51582 | 8.945 | 8.89 | 8.97 | 8.89 | 8.82 | 8.825 | 0.0161 |
| 212463_at | BE379006 | CD59 | 966 | 8.327 | 8.3 | 8.31 | 8.4 | 8.36 | 8.269 | 0.0437 |
| 212466_at | AW138902 | SPRED2 | 200734 | 3.152 | 2.81 | 2.95 | 2.98 | 3.13 | 2.935 | −0.02 |
| 212472_at | BE965029 | MICAL2 | 9645 | 6.479 | 6.27 | 6.63 | 6.31 | 6.54 | 6.389 | 0.0915 |
| 212473_s_at | BE965029 | MICAL2 | 9645 | 9.131 | 9.08 | 9.08 | 9.19 | 9.28 | 9.275 | 0.029 |
| 212523_s_at | D63480 | KIAA0146 | 23514 | 4.207 | 4.28 | 4.17 | 3.93 | 3.92 | 4.075 | −0.191 |
| 212551_at | NM_006366 | CAP2 | 10486 | 6.501 | 6.48 | 6.56 | 6.47 | 6.5 | 6.367 | 0.0252 |
| 212554_at | N90755 | CAP2 | 10486 | 6.69 | 6.52 | 6.53 | 6.46 | 6.82 | 6.686 | −0.113 |
| 212574_x_at | AC004528 | C190rf6 | 91304 | 3.675 | 3.59 | 3.75 | 3.5 | 3.59 | 3.721 | −0.011 |
| 212575_at | BF966155 | C19orf6 | 91304 | 4.21 | 4.03 | 4.09 | 3.96 | 3.82 | 4.074 | −0.094 |
| 212611_at | AV728526 | DTX4 | 23220 | 4.74 | 4.48 | 4.21 | 4.47 | 5.02 | 4.202 | −0.272 |
| 212647_at | NM_006270 | RRAS | 6237 | 8.34 | 8.27 | 8.21 | 8.27 | 8.17 | 8.305 | −0.063 |
| 212718_at | BF797555 | PAPOLA | 10914 | 9.891 | 9.92 | 9.98 | 9.83 | 10 | 9.955 | −0.003 |
| 212720_at | A1670847 | PAPOLA | 10914 | 6.397 | 6.29 | 6.41 | 6.23 | 6.18 | 6.264 | −0.02 |
| 212722_s_at | AK021780 | JMJD6 | 23210 | 5.557 | 6.08 | 6.01 | 6.06 | 5.76 | 6.095 | 0.2122 |
| 212723_at | 4K021780 | LMLD6 | 23210 | 7.967 | 7.88 | 7.93 | 8 | 8.1 | 8.084 | 0.0387 |
| 212734_x_at | AI186735 | RPL13 | 6137 | 13.11 | 13.1 | 13.1 | 13.1 | 13 | 13.11 | −0.038 |
| 212777_at | L13857 | SOS1 | 6654 | 4.05 | 3.85 | 3.83 | 3.77 | 3.64 | 3.555 | −0.154 |
| 212780_at | AA700167 | SOS1 | 6654 | 5.949 | 5.88 | 5.97 | 5.87 | 6.01 | 5.975 | 0.0068 |
| 212816_s_at | BE613178 | CBS | 875 | 6.603 | 6.6 | 6.57 | 6.43 | 6.64 | 6.595 | −0.101 |
| 212817_at | AK023253 | DNAJB5 | 25822 | 4.98 | 4.73 | 5.14 | 4.97 | 5.09 | 4.122 | 0.1997 |
| 212848_s_at | BG036668 | C9orf3 | 84909 | 5.826 | 5.72 | 5.58 | 5.9 | 5.95 | 5.88 | −0.033 |
| 212858_at | AL520675 | PAQR4 | 124222 | 3.623 | 3.54 | 3.29 | 3.42 | 3.74 | 3.635 | −0.228 |
| 212869_x_at | AI721229 | TPT1 | 7178 | 13.18 | 13.2 | 13.2 | 13.1 | 13.2 | 13.18 | −0.028 |
| 212873_at | BE349017 | HMHA1 | 23526 | 3.905 | 4.06 | 3.98 | 3.93 | 4.27 | 4.062 | −0.028 |
| 212877_at | AA284075 | KLC1 | 3831 | 6.493 | 6.5 | 6.54 | 6.48 | 6.72 | 6.722 | 0.0169 |
| 212878_s_at | AA284075 | KLC1 | 3831 | 8.431 | 8.42 | 8.44 | 8.35 | 8.45 | 8.46 | −0.033 |
| 212898_at | AB007866 | KIAA0406 | 9675 | 7.398 | 7.64 | 7.43 | 7.55 | 7.17 | 7.281 | −0.025 |
| 212910_at | W19873 | THAP11 | 57215 | 6.602 | 6.59 | 6.59 | 6.53 | 6.63 | 6.628 | −0.037 |
| 212924_s_at | N37057 | LSM4 | 25804 | 4.692 | 4.42 | 4.73 | 4.8 | 4.97 | 4.789 | 0.2107 |
| 212933_x_at | AA961748 | RPL13 | 6137 | 11.81 | 11.8 | 11.8 | 11.8 | 11.7 | 11.83 | −0.021 |
| 212944_at | AK024896 | SLCSA3 | 6526 | 7.912 | 7.75 | 7.78 | 7.78 | 7.58 | 7.553 | −0.055 |
| 212970_at | AI694303 | APBB2 | 323 | 5.695 | 5.71 | 5.61 | 5.5 | 5.83 | 5.651 | −0.146 |
| 212971_at | AI769685 | CARS | 833 | 11.18 | 11.2 | 11.2 | 11.2 | 11.3 | 11.26 | 0.0707 |
| 212972_x_at | AL080130 | APBB2 | 323 | 4.409 | 4.29 | 4.15 | 4.59 | 4.21 | 4.188 | 0.0216 |
| 212974_at | AI808958 | DENND3 | 22898 | 3.155 | 3.38 | 3.15 | 3.24 | 2.66 | 2.951 | −0.07 |
| 212975_at | AB020677 | DENND3 | 22898 | 3.948 | 4.18 | 4.22 | 4.02 | 3.9 | 4.057 | 0.0553 |
| 212985_at | BF115739 | APBB2 | 323 | 6.323 | 6.15 | 6.18 | 6.2 | 6.55 | 6.063 | −0.049 |
| 212992_at | AI935123 | AHNAK2 | 113146 | 8.982 | 8.98 | 8.99 | 9.05 | 8.95 | 9.015 | 0.0412 |
| 213010_at | AI088622 | PRKCDBP | 112464 | 6.73 | 6.7 | 6.58 | 6.62 | 6.74 | 6.756 | −0.114 |
| 213017_at | AL534702 | ABHD3 | 171586 | 6.774 | 6.83 | 6.83 | 6.78 | 6.69 | 6.649 | 0.0048 |
| 213043_s_at | AI023317 | MED24 | 9862 | 6.121 | 6.01 | 6.13 | 6.1 | 6 | 6.049 | 0.0484 |
| 213072_at | AI928387 | CYHR1 | 50625 | 4.261 | 4.05 | 4.05 | 4.05 | 4.16 | 4.032 | −0.106 |
| 213076_at | D38169 | ITPKC | 80271 | 4.077 | 4.2 | 4.1 | 4.1 | 4.16 | 3.9 | −0.039 |
| 213087_s_at | BF690020 | EEF1D | 1936 | 5.294 | 4.72 | 5.31 | 5.23 | 5.65 | 5.723 | 0.2623 |
| 213093_at | AI471375 | PRKCA | 5578 | 5.315 | 5.29 | 5.41 | 5.59 | 5.71 | 5.527 | 0.1931 |
| 213099_at | AB018302 | ANGEL1 | 23357 | 5.039 | 4.85 | 5.19 | 4.88 | 4.76 | 4.831 | 0.0919 |
| 213107_at | R59093 | TNIK | 23043 | 4.139 | 3.98 | 4.41 | 4.37 | 3.82 | 4.159 | 0.3275 |
| 213109_at | N25621 | TNIK | 23043 | 3.318 | 3.26 | 3.27 | 3.11 | 2.99 | 3.079 | −0.098 |
| 213124_at | BG538800 | ZNF473 | 25888 | 5.725 | 5.83 | 5.8 | 5.82 | 5.92 | 5.677 | 0.033 |
| 213130_at | AB032967 | 2NF473 | 25888 | 4.316 | 4.36 | 4.43 | 4.28 | 4.63 | 4.621 | 0.0166 |
| 213164_at | AI867198 | SLC5A3 | 6526 | 7.52 | 7.47 | 7.52 | 7.41 | 7.4 | 7.382 | −0.029 |
| 213167_s_at | BF982927 | SLC5A3 | 6526 | 2.909 | 2.85 | 2.94 | 2.78 | 2.82 | 2.969 | −0.02 |
| 213176_s_at | AI910869 | LTBP4 | 8425 | 4.183 | 4.33 | 4.4 | 4.31 | 3.85 | 4.04 | 0.0978 |
| 213252_at | AI739005 | SH3PXD2A | 9644 | 4.342 | 4.5 | 4.5 | 4.24 | 4.57 | 4.534 | −0.051 |
| 213268_at | Z98884 | CAMTA1 | 23261 | 2.659 | 3 | 2.85 | 2.77 | 2.89 | 2.904 | −0.023 |
| 213288_at | AI761250 | MBOAT2 | 129642 | 6.785 | 6.98 | 6.94 | 6.95 | 6.92 | 6.665 | 0.064 |
| 213302_at | AL044326 | PFAS | 5198 | 7.239 | 7.16 | 6.94 | 7.08 | 7.31 | 7.249 | −0.186 |
| 213330_s_at | BE886580 | STIP1 | 10963 | 8.702 | 8.75 | 8.67 | 8.71 | 8.52 | 8.48 | −0.033 |
| 213333_at | AL520774 | MDH2 | 4191 | 5.672 | 5.56 | 5.52 | 5.33 | 5.67 | 5.576 | −0.191 |
| 213349_at | AI934469 | TMCC1 | 23023 | 4.383 | 4.26 | 4.3 | 4.65 | 4.8 | 4.687 | 0.151 |
| 213351_s_at | AB018322 | TMCC1 | 23023 | 5.793 | 5.57 | 5.66 | 6.09 | 6.46 | 6.108 | 0.1935 |
| 213352_at | AB018322 | TMCC1 | 23023 | 3.619 | 3.99 | 3.59 | 3.67 | 4.66 | 3.867 | −0.176 |
| 213376_at | AI656706 | ZBTB1 | 22890 | 7.022 | 7.04 | 7.12 | 7.15 | 7.05 | 7.138 | 0.101 |
| 213388_at | H15535 | PDE4DIP | 9659 | 5.953 | 5.95 | 5.74 | 5.65 | 6.33 | 6.106 | −0.259 |
| 213391_at | AI669947 | DPY19L4 | 286148 | 7.708 | 7.61 | 7.71 | 7.58 | 7.66 | 7.669 | −0.009 |
| 213397_x_at | AI761728 | RNASE4 | 6038 | 4.006 | 4.01 | 3.89 | 3.84 | 4.15 | 4.275 | −0.141 |
| 213418_at | NM_002155 | HSPA6 | 3310 | 2.947 | 3.29 | 3.27 | 3.11 | 3.25 | 3.202 | 0.0696 |
| 213419_at | U62325 | APBB2 | 323 | 5.986 | 5.77 | 5.91 | 5.65 | 6.35 | 6.125 | −0.097 |
| 213422_s_at | AW888223 | MXRA8 | 54587 | 3.064 | 3.16 | 3.03 | 3.25 | 2.85 | 2.903 | 0.0234 |
| 213426_s_at | AA15011O | CAV2 | 858 | 4.142 | 3.75 | 4.07 | 4.31 | 3.86 | 3.981 | 0.2426 |
| 213445_at | D63484 | 2C3H3 | 23144 | 3.939 | 4.04 | 4.1 | 4.23 | 3.61 | 4.088 | 0.1755 |
| 213466_at | BE965869 | RAB40C | 57799 | 3.49 | 3.27 | 3.18 | 3.02 | 3.41 | 3.197 | −0.278 |
| 213481_at | N92920 | S10DA13 | 6284 | 4.195 | 3.94 | 3.91 | 4.18 | 4.23 | 4.475 | −0.022 |
| 213487_at | AI762811 | MAP2K2 | 5605 | 3.036 | 2.76 | 2.87 | 2.94 | 2.98 | 3.084 | 0.0081 |
| 213490_s_at | AT762811 | MAP2K2 | 5605 | 5.21 | 5.23 | 5.18 | 5.1 | 5.18 | 5.136 | −0.083 |
| 213492_at | X06268 | COL2A1 | 1280 | 3.013 | 3.03 | 2.9 | 3.29 | 2.75 | 3.063 | 0.0762 |
| 213509_x_at | AW157619 | CES2 | 8824 | 6.986 | 7.05 | 7.06 | 6.98 | 7.05 | 7.132 | −2E−04 |
| 213535_s_at | AA910614 | UBE2I | 7329 | 9.483 | 9.57 | 9.49 | 9.56 | 9.42 | 9.46 | 6E−05 |
| 213536_s_at | AA910614 | UBE2I | 7329 | 3.328 | 2.94 | 3.3 | 3.21 | 3.8 | 3.314 | 0.1185 |
| 213545_x_at | BE962615 | SNX3 | 8724 | 9.459 | 9.58 | 9.36 | 9.29 | 9.52 | 9.548 | −0.194 |
| 213551_x_at | AI744229 | PCGF2 | 7703 | 5.189 | 5.26 | 5.08 | 5.11 | 5.33 | 5.218 | −0.127 |
| 213559_s_at | BF223401 | ZNF467 | 168544 | 2.984 | 2.79 | 2.63 | 2.91 | 3.04 | 3.095 | −0.118 |
| 213602_s_at | AA401885 | MMP11 | 4320 | 3.321 | 3.25 | 3.42 | 3.67 | 3.19 | 3.313 | 0.2574 |
| 213608_s_at | AI220627 | SRRD | 402055 | 6.336 | 6.37 | 6.34 | 6.24 | 6.36 | 6.332 | −0.063 |
| 213636_at | AB028968 | KIAA1045 | 23349 | 2.815 | 2.87 | 2.8 | 2.86 | 2.53 | 2.591 | −0.011 |
| 213549_at | AA524053 | SFRS7 | 6432 | 8.439 | 8.41 | 8.41 | 8.42 | 8.47 | 8.462 | −0.008 |
| 213656_s_at | BF593594 | KLC1 | 3831 | 9.155 | 9.07 | 9.14 | 9.2 | 9.42 | 9.308 | 0.0548 |
| 213681_at | AW512817 | CYHR1 | 50626 | 3.952 | 3.7 | 3.89 | 3.75 | 3.84 | 3.789 | −0.008 |
| 213688_at | N25325 | CALM1 /// CALM2 /// | 801 /// 805 /// 808 | 3.559 | 3.38 | 3.62 | 3.76 | 3.59 | 3.428 | 0.2188 |
| | | CALM3 | | | | | | | | |
| 213708_s_at | N40555 | MLX | 6945 | 9.132 | 9.32 | 9.13 | 9.07 | 9.29 | 9.234 | −0.023 |
| 213741_s_at | BF575685 | KPNA1 | 3836 | 7.083 | 7.05 | 6.94 | 7.01 | 6.86 | 7.095 | −0.093 |
| 213849_s_at | AA974416 | PPP2R2B | 5521 | 3.289 | 3.28 | 3.54 | 3.11 | 3.27 | 3.2 | 0.0406 |
| 213858_at | BE350026 | ZNF250 | 58500 | 3.951 | 4.02 | 3.73 | 3.78 | 4.15 | 3.985 | −0.229 |
| 213871_s_at | AA523444 | C6orf108 | 10591 | 2.783 | 3.07 | 2.93 | 2.88 | 3.09 | 3.141 | −0.021 |
| 213889_at | AI742901 | PIGL | 9487 | 5.933 | 5.94 | 5.97 | 6.16 | 6.44 | 6.18 | 0.1268 |
| 213910_at | AW770896 | IGFBP7 | 3490 | 2.946 | 3.05 | 2.89 | 2.86 | 2.79 | 3.003 | −0.127 |
| 213917_at | BE465829 | PAX8 | 7849 | 3.106 | 3 | 2.88 | 2.93 | 2.84 | 2.841 | −0.145 |
| 213927_at | AV753204 | MAP3K9 | 4293 | 4.821 | 4.7 | 4.97 | 4.57 | 4.94 | 4.945 | 0.0063 |
| 213941_x_at | AI970731 | RPS7 | 6201 | 12.17 | 12.2 | 12.2 | 12.2 | 12.3 | 12.22 | 0.0127 |
| 213942_at | AL134303 | MEGF6 | 1953 | 3.766 | 3.42 | 3.82 | 3.37 | 3.42 | 3.818 | 0.0036 |
| 213969_x_at | BF683426 | RPL29 /// RPL29P4 | 387101 /// 6159 | 12.52 | 12.S | 12.5 | 12.5 | 12.4 | 12.49 | 0.0076 |
| 213982_s_at | BG107203 | RABGAP1L | 9910 | 6.873 | 6.8 | 6.83 | 6.75 | 6.92 | 6.862 | −0.047 |
| 213985_s_at | H45660 | C19orf6 | 91304 | 2.933 | 3.32 | 2.95 | 3.26 | 3.4 | 3.143 | −0.023 |
| 213986_s_at | AI805266 | C19orf6 | 91304 | 4.79 | 5.11 | 5.04 | 4.65 | 4.72 | 4.783 | −0.108 |
| 214026_s_at | AI860246 | SPRED2 | 200734 | 2.652 | 2.71 | 2.89 | 3.02 | 2.75 | 2.79 | 0.2754 |
| 214040_s_at | BE675337 | GSN | 2934 | 4.698 | 4.91 | 4.66 | 4.57 | 4.35 | 4.534 | −0.19 |
| 214047_s_at | AI913365 | MBD4 | 8930 | 8.454 | 8.41 | 8.46 | 8.26 | 8.44 | 8.52 | −0.075 |
| 214048_at | AI953365 | MBD4 | 8930 | 4.964 | 4.95 | 5.03 | 4.98 | 5.07 | 5.012 | 0.0478 |
| 254061_at | AI017564 | WDR67 | 93594 | 5.948 | 6.07 | 6.15 | 5.9 | 6.14 | 6.078 | 0.0229 |
| 214080_x_at | AI815793 | PRKCSH | 5589 | 7.412 | 7.44 | 7.41 | 7.45 | 7.22 | 7.44 | 0.0017 |
| 214099_s_at | AK001619 | PDE4DIP | 9659 | 4.691 | 4.69 | 4.86 | 4.77 | 4.85 | 4.749 | 0.1266 |
| 214129_at | AI821791 | PDE4DIP | 9659 | 6.244 | 5.98 | 6.08 | 5.93 | 6.51 | 6.286 | −0.111 |
| 214130_s_at | AI821791 | PDE4DIP | 9659 | 4.213 | 4.33 | 4.34 | 4.22 | 4.18 | 4.216 | 0.0114 |
| 214134_at | BF939689 | C2orf55 | 343990 | 2.958 | 2.89 | 3.01 | 3.07 | 3.06 | 2.957 | 0.1191 |
| 214141_x_at | BF033354 | SFRS7 | 6432 | 9.534 | 9.64 | 9.5 | 9.52 | 9.72 | 9.651 | −0.077 |
| 214164_x_at | BF752277 | CA12 | 771 | 7.276 | 7.34 | 7.18 | 7.35 | 7.42 | 7.261 | −0.043 |
| 214177_s_at | AI935162 | PBXIP1 | 57326 | 4.497 | 3.99 | 4.22 | 4.42 | 3.93 | 4.654 | 0.0754 |
| 214239_x_at | AI560455 | PCGF2 | 7703 | 6.884 | 6.94 | 6.93 | 7 | 6.99 | 7.112 | 0.0554 |
| 214310_s_at | AI767884 | ZFPL1 | 7542 | 4.662 | 4.78 | 5.08 | 4.83 | 4.74 | 4.656 | 0.2361 |
| 214311_at | AI767884 | ZFPL1 | 7542 | 3.109 | 3.24 | 3.12 | 2.92 | 2.9 | 3.064 | −0.153 |
| 214327_x_at | AI888178 | TPT1 | 7178 | 12.46 | 12.5 | 12.5 | 12.5 | 12.4 | 12.49 | 0.0126 |
| 214328_s_at | R01140 | HSP90AA1 | 3320 | 11.96 | 12 | 11.9 | 11.9 | 11.8 | 11.88 | −0.065 |
| 214335_at | AI669349 | RPL18 | 6141 | 3.771 | 3.22 | 3.71 | 3.71 | 3.24 | 3.46 | 0.214 |
| 214336_s_at | AI621079 | COPA | 1314 | 7.853 | 7.94 | 7.76 | 7.6 | 7.38 | 7.528 | −0.218 |
| 214337_at | AI621079 | COPA | 1314 | 2.945 | 3.25 | 3.03 | 2.86 | 3.31 | 2.83 | −0.152 |
| 214338_at | AL050381 | DNAJB12 | 54788 | 4.247 | 4.37 | 4.22 | 4.14 | 4.41 | 4.443 | −0.129 |
| 214351_x_at | AA789278 | RPL13 | 6137 | 12.21 | 12.2 | 12.1 | 12.1 | 12.2 | 12.16 | −0.1 |
| 214359_s_at | AI218219 | HSP90AB1 | 3326 | 9.692 | 9.75 | 9.65 | 9.51 | 9.12 | 9.235 | −0.14 |
| 214391_x_at | AI762344 | PTGER1 | 5731 | 3.206 | 3.04 | 3.18 | 3.25 | 3.51 | 3.236 | 0.096 |
| 214394_x_at | AI613383 | EEF1D | 1936 | 11.22 | 11.2 | 11.2 | 11.3 | 11.3 | 11.29 | 0.0465 |
| 214395_x_at | AI335509 | EEF1D | 1936 | 5.575 | 5.14 | 5.61 | 5.46 | 5.6 | 5.572 | 0.1765 |
| 214430_at | NM_000169 | GLA | 2717 | 7.146 | 7.13 | 7.21 | 7.12 | 7.29 | 7.331 | 0.0268 |
| 214482_at | NM_006977 | ZBTB25 | 7597 | 5.07 | 5.2 | 5.32 | 5.44 | 5.23 | 5.336 | 0.2441 |
| 214494_s_at | NM_005200 | SPG7 | 6687 | 6.833 | 6.9 | 6.99 | 6.92 | 6.72 | 6.872 | 0.0902 |
| 214516_at | NM_003544 | HIST1H4A /// | 121504 /// 554313 | 3.062 | 2.9 | 2.9 | 2.78 | 2.87 | 2.811 | −0.145 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 214528_s_at | NM_013951 | PAX8 | 7849 | 2.513 | 2.53 | 2.56 | 2.52 | 2.59 | 2.782 | 0.021 |
| 214536_at | NM_020427 | SLURP1 | 57152 | 3.066 | 2.55 | 3.11 | 2.86 | 2.84 | 2.719 | −0.023 |
| 214544_s_at | NM_003825 | SNAP23 | 8773 | 4.957 | 5.05 | 4.88 | 5.21 | 4.36 | 5.092 | 0.0419 |
| 214550_s_at | AFI45029 | TNPO3 | 23534 | 6.833 | 6.85 | 6.83 | 6.96 | 6.66 | 6.78 | 0.0196 |
| 214600_at | AW771935 | TFAD1 | 7003 | 5.392 | 5.34 | 5.36 | 5.33 | 5.28 | 5.394 | −0.02 |
| 234606_s_at | AJ000098 | EYA1 | 2138 | 2.988 | 2.88 | 3.28 | 2.98 | 3.12 | 2.969 | 0.2001 |
| 214634_at | AL523073 | HIST1H4A /// | 121504 /// 554313 | 3.345 | 3.29 | 3.29 | 3.34 | 3.53 | 3.337 | 0.0012 |
| | | HIST1H4B /// | /// 8294 /// 8359 /// | | | | | | | |
| | | HIST1H4C /// | 8360 /// 8361 /// | | | | | | | |
| | | HIST1H4D /// | 8362 /// 8363 /// | | | | | | | |
| | | HIST1H4E /// | 8364 /// 8365 /// | | | | | | | |
| | | HIST1H4F /// | 8366 /// 8367 /// | | | | | | | |
| | | HIST1H4H /// | 8368 /// 8370 | | | | | | | |
| | | HIST1H4I /// | | | | | | | | |
| | | HIST1H4J /// | | | | | | | | |
| | | HIST1H4K /// | | | | | | | | |
| | | HIST1H4L /// | | | | | | | | |
| | | HIST2H4A /// | | | | | | | | |
| | | HIST2H4B /// | | | | | | | | |
| | | HIST4H4 | | | | | | | | |
| 214692_s_at | AL041139 | JRK | 8629 | 4.71 | 4.87 | 4.91 | 4.93 | 4.78 | 4.976 | 0.1291 |
| 214721_x_at | AL162074 | CDC42EP4 | 23580 | 3.829 | 4.47 | 3.8 | 4.28 | 4.03 | 4.376 | −0.11 |
| 214743_at | BE046521 | CUX1 | 1523 | 6.863 | 6.92 | 6.96 | 6.91 | 7.14 | 7.09 | 0.0494 |
| 214746_s_at | BE549732 | ZNF467 | 168544 | 3.067 | 3.2 | 2.77 | 2.97 | 2.98 | 3.091 | −0.257 |
| 214748_at | US0529 | N4BP2L2 | 10443 | 4.475 | 4.58 | 4.52 | 4.57 | 4.34 | 4.303 | 0.0211 |
| 214753_3t | AW084068 | N4BP2L2 | 10443 | 7.676 | 7.75 | 7.77 | 7.74 | 7.56 | 7.68 | 0.0389 |
| 214760_at | AL049942 | 2NF337 | 26152 | 5.389 | 5.25 | 5.54 | 5.52 | 5.44 | 5.278 | 0.2304 |
| 214818_at | AF007146 | CCDC57 | 284001 | 3.567 | 3.69 | 3.57 | 3.58 | 3.65 | 3.389 | −0.051 |
| 214827_at | AL031680 | PARD6B | 84612 | 2.606 | 2.54 | 2.69 | 2.73 | 2.7 | 2.665 | 0.1383 |
| 214882_s_at | BG254869 | SFRS2 | 6427 | 9.713 | 9.59 | 9.6 | 9.66 | 9.63 | 9.765 | −0.023 |
| 214894_x_at | AK023285 | MACF1 | 23499 | 7.193 | 7.18 | 7.17 | 7.08 | 7.16 | 7.008 | −0.065 |
| 214925_s_at | AK026484 | SPTAN1 | 6709 | 4.771 | 4.46 | 4.97 | 5.02 | 4.24 | 4.417 | 0.3809 |
| 214926_at | AK026484 | SPTAN1 | 6709 | 2.919 | 2.81 | 2.68 | 2.89 | 2.6 | 2.854 | −0.079 |
| 214953_s_at | X06989 | APP | 351 | 8.352 | 8.24 | 8.48 | 8.25 | 8.45 | 8.271 | 0.0665 |
| 214969_at | AF2S1442 | MAP3K9 | 4293 | 3.057 | 3.06 | 2.94 | 3.06 | 2.78 | 3.083 | −0.058 |
| 214976_at | AI554467 | RPL13 | 6137 | 4.174 | 4.17 | 4.23 | 3.97 | 4.17 | 3.959 | −0.075 |
| 215005_at | AV723666 | NECAB2 | 54550 | 3.579 | 3.77 | 3.6 | 3.68 | 3.85 | 3.693 | −0.037 |
| 215046_at | AL133053 | C2orf67 | 151050 | 2.754 | 2.61 | 2.58 | 2.74 | 2.84 | 2.64 | −0.039 |
| 215069_at | AK025065 | NMT2 | 9397 | 3.215 | 3.33 | 3.32 | 3.29 | 3.6 | 3.256 | 0.0342 |
| 215092_s_at | AJ005683 | NFAT5 | 10725 | 5.598 | 5.5 | 5.41 | 5.67 | 5.28 | 5.426 | −0.006 |
| 215157_x_at | AI734929 | PABPC1 | 26986 | 12.66 | 12.6 | 12.6 | 12.7 | 12.7 | 12.67 | 0.0205 |
| 215184_at | AK026801 | DAPK2 | 23604 | 3.159 | 3 | 3.2 | 3.32 | 3.07 | 3.122 | 0.1817 |
| 215194_at | AF035594 | PRKCA | 5578 | 2.837 | 2.77 | 3.02 | 3.09 | 2.97 | 3 | 0.2498 |
| 215195_at | AF035594 | PRKCA | 5578 | 3.516 | 3.68 | 3.46 | 3.63 | 3.71 | 3.711 | −0.05 |
| 215205_x_at | S83390 | NCOR2 | 9612 | 3 | 2.84 | 2.89 | 2.69 | 3.16 | 2.887 | −0.13 |
| 215222_x_at | AK023406 | MACF1 | 23499 | 7.084 | 6.94 | 7.01 | 6.87 | 6.79 | 6.91 | −0.072 |
| 215231_at | AU144309 | PRKAG2 | 51422 | 3.273 | 3.23 | 3.33 | 3.21 | 3.14 | 3.248 | 0.014 |
| 215233_at | AA351360 | JMJD6 | 23210 | 3.043 | 3.21 | 3.28 | 2.88 | 3.07 | 2.869 | −0.047 |
| 215235_at | AL110273 | SPTAN1 | 6709 | 5.958 | 5.68 | 6.11 | 6.09 | 6.24 | 5.91 | 0.2824 |
| 215240_at | AI189839 | ITGB3 | 3690 | 2.691 | 2.97 | 2.74 | 2.63 | 2.76 | 2.906 | −0.15 |
| 215270_at | U94354 | LFNG | 3955 | 2.897 | 2.96 | 2.88 | 2.96 | 2.85 | 2.792 | 0.0038 |
| 215337_at | AK022508 | MED24 | 9862 | 3.262 | 3.18 | 3.16 | 2.98 | 3.22 | 3.328 | −0.152 |
| 215342_s_at | AB019490 | RABGAP1L | 9910 | 4.506 | 4.66 | 4.74 | 4.61 | 4.74 | 4.594 | 0.0434 |
| 215374_at | AK024849 | PAPOLA | 10914 | 3.55 | 3.36 | 3.18 | 3.38 | 3.32 | 3.312 | −0.174 |
| 215377_at | AK024129 | CTBP2 | 1488 | 3.376 | 3.33 | 3.66 | 3.41 | 3.53 | 3.516 | 0.1845: |
| 215548_s_at | AB020724 | SCFD1 | 23256 | 8.918 | 8.97 | 8.79 | 8.88 | 8.87 | 8.836 | −0.114 |
| 215575_at | AU157078 | PDE4DIP | 9659 | 3.056 | 2.77 | 3.22 | 3.12 | 3.04 | 3.269 | 0.26 |
| 215584_at | AK022679 | HECW1 | 23072 | 3.475 | 3.32 | 3.26 | 3.38 | 3.12 | 3.335 | −0.077 |
| 215517_at | AU145711 | LOC26010 | 26010 | 2.915 | 2.81 | 2.86 | 2.71 | 2.97 | 2.865 | −0.075 |
| 215631_s_at | AL0S0G08 | BRMS1 | 25855 | 6.705 | 6.59 | 6.55 | 6.53 | 6.52 | 6.752 | −0.11 |
| 215688_at | AL359931 | RASGRF1 | 5923 | 3.323 | 3.31 | 3.14 | 3.32 | 2.92 | 3.158 | −0.084 |
| 215728_at | AL031848 | ACOT7 | 11332 | 7.335 | 7.41 | 7.31 | 7.54 | 6.97 | 7.572 | 0.0552 |
| 215732_s_at | AK023924 | DTX2 /// | 100134197 // | 3.791 | 3.57 | 3.86 | 4.17 | 3.54 | 3.892 | 0.33 |
| | | LOC100134197 | 113878 | | | | | | | |
| 215743_at | AL134483 | NMT2 | 9397 | 3.186 | 3.46 | 3.26 | 3.46 | 3.16 | 3.32 | 0.0376 |
| 215852_x_at | AK022023 | C20orftL17 | 140710 | 2.836 | 2.91 | 2.86 | 2.99 | 2.3 | 2.959 | 0.0484 |
| 215867_x_at | AL050025 | CA12 | 771 | 7.222 | 7.32 | 7.15 | 7.19 | 7.39 | 7.238 | −0.102 |
| 215912_at | AA758795 | GNAO1 | 2775 | 3.211 | 3.3 | 3.3 | 3.38 | 3.24 | 3.221 | 0.0888 |
| 215938_s_at | AK001290 | PLA2G6 | 8398 | 3.018 | 3.04 | 3 | 2.98 | 3.08 | 2.935 | −0.039 |
| 215980_s_at | AF052128 | IGHMBP2 | 3508 | 3.669 | 3.8 | 4.02 | 4.09 | 3.82 | 4.067 | 0.3163 |
| 215991_s_at | AU121504 | KIAA0090 | 23065 | 2.978 | 2.89 | 3.35 | 3.04 | 3.14 | 3.1 | 0.2556 |
| 216105_x_at | X86428 | PPP2R4 | 5524 | 4.731 | 4.74 | 4.75 | 4.59 | 4.6 | 4.986 | −0.064 |
| 216261_at | AI151479 | ITGB3 | 3690 | 3.142 | 2.96 | 2.91 | 2.94 | 3.06 | 2.923 | −0.129 |
| 216309_x_at | AF072467 | JPX | 8629 | 5.108 | 5.31 | 5.26 | 4.96 | 5.08 | 5.116 | −0.099 |
| 216364_s_at | AJ001550 | AFF2 | 2334 | 2.547 | 2.69 | 2.67 | 2.8 | 2.58 | 2.619 | 0.1148 |
| 216382_s_at | U80756 | MLL2 | 8085 | 3.829 | 3.84 | 3.72 | 3.23 | 3.66 | 3.631 | −0.364 |
| 216407_at | U25801 | VAC14 | 55697 | 3.453 | 3.7 | 3.85 | 3.9 | 3.99 | 3.894 | 3.3008 |
| 216501_at | U25801 | VAC14 | 55697 | 2.875 | 2.75 | 2.95 | 2.73 | 2.7 | 2.775 | 0.033 |
| 216520_s_at | AF072098 | TPT1 | 7178 | 13.1 | 13.1 | 13.1 | 13.1 | 13 | 13.05 | −0.005 |
| 216533_at | AL122056 | PCCA | 5095 | 2.722 | 2.69 | 2.85 | 2.76 | 2.5 | 2.562 | 0.102 |
| 216570_x_at | AL096829 | LOC100131713 /// | 100131713 /// | 9.897 | 9.86 | 9.93 | 9.84 | 3.46 | 9.776 | 0.0089 |
| | | LOC283412 /// | 283412 /// 284064 | | | | | | | |
| | | LOC284064 /// | /// 387101 /// | | | | | | | |
| | | LOC391019 /// | 391019 /// 6159 /// | | | | | | | |
| | | LOC643531 /// | 643531 /// 647285 | | | | | | | |
| | | LOC647285 /// | /// 728820 | | | | | | | |
| | | LOC728820 /// RPL29 | | | | | | | | |
| | | /// RPL29P4 | | | | | | | | |
| 216624_s_at | Z69744 | MLL | 4297 | 3.084 | 3.32 | 3.28 | 3.42 | 3.14 | 3.244 | 0.1504 |
| 216678_at | AK000773 | IFT122 | 55764 | 4.103 | 4.3 | 4.26 | 4.19 | 3.89 | 4.341 | 0.0224 |
| 216697_at | AL161955 | TRIO | 7204 | 2.993 | 2.86 | 2.76 | 2.96 | 2.83 | 2.794 | −0.062 |
| 216700_at | AL161955 | TRIO | 7204 | 3.252 | 3.27 | 3.3 | 2.97 | 3.41 | 3.083 | −0.128 |
| 216747_at | AK024871 | APBB2 | 323 | 3.384 | 3.36 | 3.19 | 3.38 | 3.14 | 3.193 | −0.087 |
| 216750_at | AK024871 | APBB2 | 323 | 3.435 | 3.36 | 3.19 | 3.21 | 2.83 | 2.868 | −0.198 |
| 216845_x_at | U80756 | MLL2 | 8085 | 3.279 | 3.38 | 3.42 | 3.57 | 3.03 | 3.131 | 0.1695 |
| 216867_s_at | X03795 | PDGFA | 5154 | 4.778 | 5.03 | 4.91 | 4.71 | 5.22 | 5.157 | −0.096 |
| 216880_at | Y15571 | RAD51L1 | 5890 | 4.389 | 4.45 | 4.33 | 4.52 | 4.67 | 4.568 | 0.0046 |
| 216944_x_at | U23850 | ITPR1 | 3708 | 3.511 | 3.62 | 3.68 | 3.25 | 3.54 | 3.572 | −0.103 |
| 216952_s_at | M94363 | LMNB2 | 84823 | 5.29 | 5.25 | 5.37 | 5.02 | 5.46 | 5.057 | −0.078 |
| 216971_s_at | 254367 | PLEC1 | 5339 | 4.559 | 4.46 | 4.65 | 4.35 | 4.34 | 4.397 | −0.013 |
| 216988_s_at | L48722 | PTP4A2 | 8073 | 9.208 | 9.11 | 9.14 | 9.17 | 9.2 | 9.186 | −1E−03 |
| 217005_at | M28219 | LDLR | 3949 | 3.097 | 3.26 | 3.3 | 3.46 | 3.32 | 3.18 | 0.1992 |
| 217025_s_at | AL110225 | DBN1 | 1627 | 4.14 | 3.86 | 4.13 | 4.05 | 4.2 | 4.132 | 0.0917 |
| 217103_at | M28219 | LDLR | 3949 | 2.816 | 2.83 | 2.98 | 3.08 | 2.88 | 3.025 | 0.2084 |
| 217118_s_at | AK025608 | C22orf9 | 23313 | 6.638 | 6.69 | 6.67 | 6.49 | 6.86 | 6.663 | −0.087 |
| 217124_at | AL136792 | IQCE | 23288 | 3.321 | 3.16 | 3.21 | 3.37 | 3.3 | 3.194 | 0.0472 |
| 217144_at | X04801 | LOC648390 /// | 6233 /// 648390 /// | 5.818 | 5.91 | 5.66 | 5.7 | 5.28 | 5.351 | −0.188 |
| | | RPS27A /// UBB /// | 7314 /// 7316 | | | | | | | |
| | | UBC | | | | | | | | |
| 217146_at | AF072468 | JRK | 8629 | 3.006 | 3.06 | 3.19 | 3.06 | 2.99 | 3.104 | 0.0976 |
| 217173_s_at | S70123 | LDLR | 3949 | 5.668 | 5.88 | 6.08 | 5.83 | 5.63 | 5.714 | 0.1825 |
| 217174_s_at | AL078616 | APC2 | 10297 | 2.959 | 3.02 | 2.87 | 2.82 | 2.85 | 2.968 | −0.142 |
| 217183_at | S70123 | LDLR | 3949 | 3.085 | 3.14 | 2.39 | 3.42 | 3.22 | 2.972 | 0.2902 |
| 217262_s_at | BC000059 | CELSR1 | 9620 | 3.941 | 3.02 | 2.92 | 2.81 | 3.37 | 2.83 | −0.114 |
| 217299_s_at | AK001017 | NBN | 4683 | 6.248 | 6.1 | 6.54 | 6.29 | 6.22 | 6.09 | 0.2375 |
| 217356_s_at | S81916 | PGR1 | 5230 | 10.18 | 10.2 | 10.1 | 10.1 | 9.84 | 10.03 | −0.071 |
| 217383_at | S81916 | PGK1 | 5230 | 4.569 | 4.23 | 4.49 | 4.3 | 4.31 | 4.38 | −0.002 |
| 217404_s_at | X16468 | COL2A1 | 1280 | 2.746 | 2.85 | 2.85 | 2.96 | 2.83 | 2.953 | 0.106 |
| 217432_s_at | AF179281 | IDS | 3423 | 4.619 | 4.33 | 4.26 | 4.39 | 4.32 | 4.52 | −0.151 |
| 217466_x_at | L48784 | RP52 | 6187 | 10.13 | 10.2 | 10.1 | 10.1 | 9.96 | 10.05 | −0.011 |
| 217489_s_at | S72848 | IL6R | 3570 | 2.944 | 3.46 | 3.16 | 3.02 | 3.12 | 3.179 | −0.109 |
| 217500_at | R27378 | TIAL1 | 7073 | 3.293 | 3.1 | 3.07 | 3.21 | 2.93 | 3.221 | −0.057 |
| 217508_s_at | BE783279 | C18orf25 | 147339 | 5.046 | 5.14 | 4.63 | 5.08 | 5.41 | 5.008 | −0.236 |
| 217539_at | W28849 | C18orf25 | 147339 | 2.764 | 2.28 | 2.8 | 2.86 | 2.91 | 2.848 | 0.0546 |
| 217608_at | AW408767 | SFRS12IP1 | 285672 | 4.602 | 4.58 | 4.56 | 4.77 | 4.81 | 4.684 | 0.0758 |
| 217618_x_at | AW007988 | HUS1 | 3364 | 5.196 | 5.17 | 5.32 | 5.03 | 5.11 | 5.097 | −0.009 |
| 217622_at | AA018187 | RHBDD3 | 25807 | 5.228 | 5.28 | 4.85 | 4.94 | 5.12 | 5.255 | −0.361 |
| 217635_s_at | AA769006 | POLG | 5428 | 5.101 | 5.28 | 5.42 | 5.36 | 5.39 | 5.221 | 0.1982 |
| 217636_at | AA769006 | POLG | 5428 | 3.046 | 2.78 | 3.27 | 3.08 | 3.03 | 2.926 | 0.2652 |
| 217669_s_at | AW451230 | AKAP6 | 9472 | 3.13 | 3.24 | 3.06 | 3.46 | 3.28 | 3.178 | 0.078 |
| 217686_at | BF222916 | PTPN1 | 5770 | 3.402 | 3.42 | 3.36 | 3.36 | 3.28 | 3.305 | −0.049 |
| 217689_at | BG109555 | PTPN1 | 5770 | 2.975 | 2.99 | 2.71 | 2.93 | 3.15 | 2.74 | −0.164 |
| 217722_s_at | NM_016645 | NGRN | 51335 | 10.24 | 10.3 | 10.2 | 10.3 | 10.2 | 10.09 | −0.028 |
| 217745_s_at | NM_025146 | NAT13 | 80218 | 10.02 | 9.94 | 10 | 9.96 | 9.99 | 10 | 0.019 |
| 217752_s_at | NM_018235 | CNDP2 | 55748 | 8.929 | 8.89 | 8.96 | 8.89 | 8.91 | 9.176 | 0.0168 |
| 217756_x_at | NM_005770 | SERF2 | 10169 | 9.601 | 9.65 | 9.49 | 9.52 | 9.5 | 9.457 | −0.12 |
| 217774_s_at | NM_016404 | HSPC152 | 51504 | 11.22 | 11.2 | 11.2 | 11.2 | 11.1 | 11.11 | −0.016 |
| 217779_s_at | NM_017761 | LOC100132235 /// | 100132235 /// 55629 | 9.145 | 9.29 | 9.28 | 9.13 | 9.36 | 9.38 | −0.011 |
| | | PNRC2 | | | | | | | | |
| 217786_at | NM_006109 | PRMT5 | 10419 | 8.949 | 8.94 | 8.95 | 8.87 | 8.92 | 8.951 | −0.032 |
| 217793_at | AL575337 | RAB11B | 9230 | 3.517 | 3.54 | 3.69 | 3.49 | 3.7 | 3.732 | 0.0664 |
| 217830_s_at | AL109658 | NSFL1C | 55968 | 5.657 | 5.44 | 5.75 | 5.51 | 5.66 | 5.699 | 0.0812 |
| 217831_s_at | NM_016143 | NSFL1C | 55968 | 6.226 | 6.17 | 6.19 | 6.08 | 6.32 | 6.324 | −0.06 |
| 217868_s_at | NM_016025 | METTL9 | 51108 | 9.619 | 9.67 | 9.61 | 9.65 | 9.71 | 9.658 | −0.012 |
| 217875_s_at | NM_020182 | PMEPA1 | 56937 | 4.749 | 4.67 | 4.62 | 4.5 | 4.7 | 4.701 | −0.146 |
| 217903_at | NM_013403 | STRN4 | 29888 | 4.695 | 4.77 | 4.74 | 4.84 | 4.99 | 5.056 | 0.0567 |
| 217907_at | NM_014161 | MRPL18 | 29074 | 9.678 | 9.79 | 9.78 | 9.73 | 9.56 | 9.7 | 0.0215 |
| 217909_s_at | BF056105 | MLX | 6945 | 7.784 | 7.98 | 7.75 | 7.89 | 7.63 | 7.877 | −0.057 |
| 217910_x_at | NM_013383 | MLX | 6945 | 8.608 | 8.69 | 8.6 | 8.73 | 8.86 | 8.645 | 0.0132 |
| 217911_s_at | NM_004281 | BAG3 | 9531 | 8.16 | 8.06 | 8.04 | 8.17 | 8.17 | 8.113 | −0.006 |
| 217924_at | AL523965 | C6orf106 | 64771 | 4.463 | 4.41 | 4.23 | 4.46 | 4.89 | 4.223 | −0.09 |
| 217925_s_at | NM_022758 | C6orf106 | 64771 | 5.48 | 5.5 | 5.79 | 5.78 | 5.55 | 5.733 | 0.2917 |
| 217943_s_at | NM_018067 | MAP7D1 | 55700 | 6.29 | 6.13 | 6.32 | 6.08 | 6.37 | 6.482 | −0.005 |
| 217950_at | NM_015953 | NOSIP | 51070 | 7.293 | 7.53 | 7.29 | 7.47 | 7.37 | 7.354 | −0.028 |
| 217969_at | NM_013265 | C11orf2 | 738 | 7.286 | 7.35 | 7.37 | 7.4 | 7.55 | 7.461 | 0.0708 |
| 217980_s_at | NM_017840 | MRPL16 | 54948 | 8.277 | 8.43 | 8.31 | 8.36 | 8.35 | 8.382 | −0.02 |
| 218016_s_at | NM_018119 | POLR3E | 55718 | 7.343 | 7.32 | 7.22 | 7.29 | 7.24 | 7.311 | −0.083 |
| 218018_at | AW449022 | PDXK | 8566 | 7.706 | 7.63 | 7.63 | 7.6 | 7.87 | 7.787 | −0.053 |
| 218019_s_at | NM_021941 | PDXK | 8566 | 6.43 | 6.34 | 6.55 | 6.41 | 6.76 | 6.445 | 0.0942 |
| 218022_at | NM_016440 | VRX3 | 51231 | 6.826 | 6.82 | 7.04 | 7 | 7.23 | 7.058 | 0.1957 |
| 218023_s_at | NM_016605 | FAM53C | 51307 | 6.055 | 6.13 | 6.11 | 6.22 | 6.17 | 6.207 | 0.071 |
| 218062_x_at | NM_012121 | CDC42EP4 | 23580 | 4.353 | 4.81 | 4.34 | 4.76 | 4.68 | 4.48 | −0.033 |
| 218063_s_at | AF099664 | CDC42EP4 | 23580 | 3.069 | 3.04 | 3.06 | 3.01 | 2.87 | 2.772 | −0.02 |
| 218074_at | NM_016062 | FAM96B | 51647 | 9.946 | 9.99 | 9.86 | 9.96 | 9.94 | 10.03 | −0.062 |
| 218099_at | NM_018469 | TEX2 | 55852 | 6.338 | 6.43 | 6.6 | 6.52 | 6.6 | 6.45 | 0.1728 |
| 218132_s_at | NM_024075 | TSEN34 | 79042 | 7.619 | 7.81 | 7.72 | 7.64 | 7.79 | 7.806 | −0.031 |
| 218136_s_at | NM_018579 | SLC25A37 | 51312 | 5.372 | 5.91 | 5.62 | 5.71 | 5.58 | 5.339 | 0.0252 |
| 218138_at | NM_018848 | MKKS | 8195 | 8.188 | 8.32 | 8.24 | 8.28 | 8.18 | 8.201 | 0.0005 |
| 218141_at | NM_022066 | UBE2O | 63893 | 4.291 | 4.02 | 4.28 | 4.17 | 4.28 | 3.999 | 0.0673 |
| 218145_at | NM_021158 | TRIB3 | 57761 | 11.26 | 11.2 | 11.2 | 11.3 | 11.4 | 11.31 | 0.0156 |
| 218148_at | NM_025082 | CENPT | 80152 | 3.232 | 3.24 | 3.23 | 3.46 | 3.17 | 3.128 | 0.1086 |
| 218169_at | NM_018052 | VAC14 | 55697 | 4.973 | 4.64 | 4.79 | 4.91 | 4.82 | 4.923 | 0.0416 |
| 218181_s_at | NM_017792 | MAP4K4 | 9448 | 7.826 | 7.95 | 7.83 | 7.79 | 8 | 7.866 | −0.079 |
| 218195_at | NM_024573 | C6orf211 | 79624 | 8.018 | 7.89 | 7.99 | 7.91 | 7.83 | 7.921 | −0.001 |
| 218197_s_at | NM_018002 | OXR1 | 55074 | 7.45 | 7.4 | 7.55 | 7.52 | 7.51 | 7.532 | 0.1126 |
| 218233_s_at | NM_017601 | PRICKLE4 /// TOMM6 | 100188893 /// 29964 | 10.84 | 11 | 10.8 | 10.9 | 11 | 11.01 | −0.092 |
| 218235_s_at | NM_016037 | UTP11L | 51118 | 9.477 | 9.53 | 9.42 | 9.45 | 9.58 | 9.606 | −0.068 |
| 218246_at | NM_024544 | MUL1 | 79594 | 5.73 | 5.41 | 5.79 | 5.42 | 5.54 | 5.645 | 0.0333 |
| 218265_at | NM_024077 | SEC1SBP2 | 79048 | 4.935 | 4.94 | 4.93 | 4.78 | 5.03 | 5.078 | −0.081 |
| 218270_at | NM_024540 | MRPL24 | 79590 | 8.028 | 8.15 | 8.03 | 8.15 | 7.88 | 8.174 | 0.0036 |
| 218292_s_at | NM_016203 | PRKAG2 | 51422 | 5.102 | 5.24 | 5.15 | 5.01 | 5.53 | 5.564 | −0.087 |
| 218321_x_at | NM_016086 | STYXL1 | 51657 | 8.445 | 8.5 | 8.54 | 8.35 | 8.58 | 8.582 | −0.024 |
| 218328_at | NM_016035 | COQ4 | 51117 | 6.224 | 5.98 | 6.17 | 6 | 6.15 | 6.281 | −0.018 |
| 218343_s_at | NM_012086 | GTF3C3 | 9330 | 7.002 | 6.97 | 6.99 | 7.1 | 7.04 | 6.987 | 0.0556 |
| 218347_at | NM_018264 | TYW1 | 55253 | 6.807 | 6.82 | 6.93 | 7.07 | 6.88 | 7.002 | 0.1872 |
| 218364_at | NM_017724 | LRRFIP2 | 9209 | 6.868 | 6.87 | 6.94 | 6.78 | 6.89 | 7.035 | −0.01 |
| 218402_s_at | NM_022081 | HPS4 | 89781 | 4.269 | 4.59 | 4.17 | 4.27 | 4.2 | 3.948 | −0.214 |
| 218427_at | NM_006643 | SDCCAG3 | 10807 | 6.971 | 6.97 | 7.01 | 6.84 | 6.88 | 7.091 | −0.041 |
| 218431_at | NM_022067 | C14orf133 | 63894 | 6.433 | 6.54 | 6.66 | 6.46 | 6.46 | 6.502 | 0.0737 |
| 218480_at | NM_021831 | AGBL5 | 60509 | 5.325 | 5.45 | 5.32 | 5.02 | 5.23 | 5.18 | −0.219 |
| 218482_at | NM_020189 | ENY2 | 56943 | 10.08 | 10.2 | 10.1 | 10.1 | 10.2 | 10.14 | −0.053 |
| 218500_at | NM_016647 | C8orf55 | 51337 | 3.465 | 3.74 | 3.66 | 3.39 | 3.82 | 3.765 | −0.079 |
| 218543_s_at | NM_022750 | PARP12 | 64761 | 6.928 | 6.84 | 6.97 | 6.84 | 7.06 | 7.086 | 0.0216 |
| 218555_at | NM_013366 | ANAPC2 | 29882 | 4.741 | 4.58 | 4.41 | 4.52 | 4.89 | 4.196 | −0.193 |
| 218561_s_at | NM_020408 | LYRM4 | 57128 | 7.607 | 7.53 | 7.58 | 7.56 | 7.7 | 7.791 | 0.0065 |
| 218566_s_at | NM_012124 | CHORDC1 | 26973 | 7.93 | 7.91 | 7.89 | 8.04 | 7.95 | 7.989 | 0.049 |
| 218578_at | NM_024529 | CDC73 | 79577 | 7.388 | 7.28 | 7.14 | 7.1 | 7.16 | 7.278 | −0.217 |
| 218584_at | NM_024549 | TCTN1 | 79600 | 5.329 | 5.34 | 5.35 | 5.41 | 5.3 | 5.226 | 0.045 |
| 218596_at | NM_018201 | TBC1D13 | 54662 | 3.986 | 4.19 | 4.07 | 4.02 | 4.21 | 4.099 | −0.04 |
| 218677_at | NM_020672 | S100A14 | 57402 | 8.251 | 8.23 | 8.37 | 8.29 | 8.11 | 8.174 | 0.0785 |
| 218678_at | NM_024609 | NES | 10763 | 3.496 | 3.46 | 3.42 | 3.08 | 3.16 | 3.246 | −0.229 |
| 218680_x_at | NM_016400 | HYPK | 25764 | 8.725 | 8.84 | 8.78 | 8.67 | 8.73 | 8.842 | −0.061 |
| 218763_at | NM_016930 | STX18 | 53407 | 7.633 | 7.45 | 7.36 | 7.26 | 7.61 | 7.65 | −0.234 |
| 218767_at | NM_020385 | REXO4 | 57109 | 5.561 | 5.72 | 5.54 | 5.52 | 5.76 | 5.698 | −0.113 |
| 218810_at | NM_025079 | ZC3H12A | 80149 | 4.97 | 5.09 | 5.19 | 5.36 | 4.7 | 5.123 | 0.2409 |
| 218818_at | NM_004468 | PHL3 | 2275 | 3.724 | 3.54 | 3.52 | 3.58 | 3.47 | 3.293 | −0.08 |
| 218830_at | NM_016093 | RPL26L1 | 51121 | 9.754 | 9.82 | 9.79 | 9.83 | 9.79 | 9.808 | 0.0211 |
| 218846_at | NM_004830 | MED23 | 9439 | 6.936 | 6.89 | 7.01 | 7.09 | 7.25 | 6.978 | 0.1358 |
| 218847_at | NM_006548 | IGF2BP2 | 10644 | 9.312 | 9.34 | 9.32 | 9.41 | 9.55 | 9.417 | 0.0386 |
| 218850_s_at | NM_014240 | LIMD1 | 8994 | 3.165 | 3.26 | 3.26 | 3.29 | 3.49 | 3.439 | 0.0581 |
| 218914_at | NM_015997 | C1orf66 | 51093 | 6 | 5.94 | 6.01 | 5.97 | 6.19 | 6.274 | 0.0188 |
| 218954_s_at | AF298153 | BRF2 | 55290 | 4.688 | 4.54 | 4.42 | 4.4 | 4.43 | 4.222 | −0.209 |
| 218955_at | NM_018310 | BRF2 | 55290 | 5.146 | 5.15 | 5.14 | 5.19 | 5.33 | 5.099 | 0.0123 |
| 218965_s_at | NM_022830 | TUT1 | 64852 | 3.994 | 3.53 | 3.53 | 3.75 | 3.5 | 3.58 | −0.121 |
| 218966_at | NM_018728 | MYO5C | 55930 | 6.776 | 6.62 | 6.74 | 6.75 | 6.59 | 6.588 | 0.0421 |
| 218978_s_at | NM_018586 | SLC25A37 | 51312 | 4.466 | 3.85 | 4.08 | 4.44 | 3.81 | 3.898 | 0.0962 |
| 218991_at | NM_022070 | HEATR6 | 63897 | 7.189 | 7.37 | 7.29 | 7.29 | 7.21 | 7.307 | 0.0084 |
| 219038_at | NM_024657 | MORC4 | 79710 | 6.922 | 6.91 | 6.94 | 6.87 | 6.82 | 6.759 | −0.008 |
| 219050_s_at | NM_014205 | ZNHIT2 | 741 | 3.922 | 3.93 | 3.85 | 3.9 | 3.82 | 4.163 | −0.053 |
| 219062_s_at | NM_017742 | ZCCHC2 | 54877 | 5.587 | 5.74 | 5.91 | 5.88 | 5.86 | 5.794 | 0.2294 |
| 219076_s_at | NM_018663 | PXMP2 | 5827 | 7.119 | 7.31 | 7.11 | 7.1 | 7.16 | 7.31 | −0.111 |
| 219107_at | NM_021948 | BCAN | 63827 | 3.673 | 3.62 | 3.36 | 3.55 | 3.28 | 3.475 | −0.195 |
| 219128_at | NM_017880 | C2orf42 | 54980 | 6.48 | 6.51 | 6.36 | 6.41 | 6.61 | 6.594 | −0.11 |
| 219156_at | NM_018373 | SYNJ2BP | 55333 | 5.934 | 5.74 | 5.73 | 5.48 | 5.83 | 5.783 | −0.229 |
| 219172_at | NM_024954 | UBTD1 | 80019 | 3.344 | 3.52 | 3.33 | 3.54 | 3.54 | 3.336 | 0.0057 |
| 219175_s_at | NM_017836 | SLC41A3 | 54946 | 6.265 | 6.23 | 6.32 | 6.24 | 6.1 | 6.122 | 0.0308 |
| 219193_at | NM_018034 | WDR70 | 55100 | 7.127 | 6.96 | 7.24 | 6.98 | 7.21 | 7.099 | 0.0642 |
| 219215_s_at | NM_017767 | SLC39A4 | 55630 | 6.694 | 6.62 | 6.63 | 6.77 | 7.1 | 7.169 | 0.0435 |
| 219217_at | NM_024678 | NARS2 | 79731 | 7.358 | 7.45 | 7.4 | 7.39 | 7.44 | 7.528 | −0.009 |
| 219221_at | NM_024724 | ZBTB38 | 253461 | 7.54 | 7.45 | 7.65 | 7.4 | 7.59 | 7.643 | 0.0315 |
| 219227_at | NM_024565 | CCNJL | 79616 | 3.747 | 3.73 | 3.56 | 3.8 | 3.77 | 3.429 | −0.062 |
| 219354_at | NM_018316 | KLHL26 | 55295 | 4.355 | 4.75 | 4.63 | 4.74 | 4.54 | 4.268 | 0.1332 |
| 219357_at | NM_014027 | GTPBP1 | 9567 | 6.29 | 6.3 | 6.45 | 6.3 | 6.6 | 6.347 | 0.0801 |
| 219435_at | NM_025099 | C17orf68 | 80169 | 4.618 | 4.44 | 4.39 | 4.55 | 4.85 | 4.813 | −0.058 |
| 219456_s_at | AW027923 | RIN3 | 79890 | 3.159 | 3.05 | 2.93 | 3.02 | 3.07 | 2.959 | −0.129 |
| 219457_s_at | NM_024832 | RIN3 | 79890 | 3.403 | 3.22 | 3.29 | 3.58 | 3.58 | 3.281 | 0.1259 |
| 219459_at | NM_018082 | POLR3B | 55703 | 6.743 | 6.89 | 7.06 | 6.99 | 7.27 | 7.233 | 0.2045 |
| 219468_s_at | NM_017949 | CUEDC1 | 404093 | 3.657 | 3.58 | 3.73 | 3.63 | 3.89 | 3.944 | 0.0566 |
| 219475_at | NM_013370 | OSGIN1 | 29948 | 3.751 | 3.3 | 3.15 | 3.58 | 3.3 | 3.32 | −0.159 |
| 219489_s_at | NM_017821 | NXN | 64359 | 9.592 | 9.65 | 9.62 | 9.49 | 9.82 | 9.702 | −0.061 |
| 219495_s_at | NM_013256 | ZNF180 | 7733 | 4.994 | 4.96 | 4.82 | 4.59 | 5.06 | 5.053 | −0.269 |
| 219500_at | NM_013246 | CLCF1 | 23529 | 4.854 | 5.15 | 5.1 | 4.93 | 5.04 | 5.165 | 0.0132 |
| 219513_s_at | NM_005490 | SH2D3A | 10045 | 2.764 | 2.88 | 2.63 | 2.89 | 2.98 | 2.832 | −0.06 |
| 219543_at | NM_022129 | PBLD | 64081 | 3.387 | 3.37 | 3.64 | 3.79 | 3.78 | 3.488 | 0.3334 |
| 219572_at | NM_037954 | CADPS2 | 93664 | 3.499 | 3.36 | 3.41 | 3.62 | 3.46 | 3.181 | 0.0848 |
| 219577_s_at | NM_019112 | ABCA7 | 10347 | 3.119 | 3.27 | 3.47 | 3.14 | 3.32 | 3.191 | 0.1085 |
| 219610_at | NM_022448 | RGNEF | 64283 | 4.738 | 4.93 | 4.94 | 4.96 | 5.02 | 4.952 | 0.115 |
| 219631_at | NM_024937 | LRP12 | 29967 | 6.225 | 6.24 | 6.34 | 5.21 | 6.31 | 6.215 | 0.0433 |
| 219677_st | NM_025106 | SPSB1 | 80176 | 4.604 | 4.87 | 4.53 | 4.7 | 4.88 | 4.802 | −0.119 |
| 219692_at | NM_024507 | KREMEN2 | 79412 | 3.685 | 4.07 | 3.56 | 3.8 | 3.61 | 3.745 | −0.195 |
| 219710_at | NM_024577 | SH3TC2 | 79628 | 3.827 | 3.86 | 4.53 | 3.9 | 4.19 | 4.075 | 0.3728 |
| 239742_at | NM_030567 | PRR7 | 80758 | 3.403 | 3.3 | 3.46 | 3.35 | 3.52 | 3.431 | 0.0516 |
| 219758_at | NM_024926 | TTC26 | 79989 | 4.916 | 4.75 | 4.67 | 4.57 | 4.55 | 4.259 | −0.216 |
| 219783_at | NM_017877 | C2orf18 | 54978 | 4.949 | 4.85 | 4.76 | 4.77 | 4.73 | 4.802 | −0.129 |
| 219784_at | NM_024735 | FBXO31 | 79791 | 5.386 | 5.19 | 5.05 | 5.23 | 5.37 | 5.507 | −0.146 |
| 219785_s_at | NM_024735 | FBXO31 | 79791 | 5.471 | 5.73 | 5.51 | 5.84 | 6.3 | 5.911 | 0.0765 |
| 219794_at | NM_018289 | VPS53 | 55275 | 3.132 | 3.2 | 3.08 | 3.13 | 3.2 | 3.129 | −0.061 |
| 219801_at | NM_030580 | ZNF34 | 80778 | 3.509 | 3.5 | 3.69 | 3.73 | 3.82 | 3.804 | 0.2049 |
| 219816_s_at | NM_018107 | RBM23 | 55147 | 6.728 | 6.8 | 6.87 | 6.82 | 6.73 | 6.627 | 0.0778 |
| 219830_at | NM_030665 | RAI1 | 10743 | 3.034 | 2.89 | 3.26 | 3.24 | 3.19 | 2.935 | 0.2863 |
| 239831_at | NM_016508 | CDKL3 | 51265 | 6.01 | 5.9 | 6.09 | 6.16 | 6.28 | 6.153 | 0.17 |
| 219842_at | NM_019087 | ARL15 | 54622 | 3.234 | 3.14 | 3.15 | 3.05 | 3.19 | 3.099 | −0.086 |
| 219862_s_at | NM_012336 | NARF | 26502 | 7.47 | 7.61 | 7.47 | 7.5 | 7.52 | 7.501 | −0.057 |
| 219899_x_at | NM_014434 | NDOR1 | 27158 | 3.397 | 3.55 | 3.39 | 3.33 | 3.42 | 3.577 | −0.117 |
| 219901_at | NM_018351 | FGD6 | 55785 | 5.547 | 5.38 | 5.55 | 5.43 | 5.61 | 5.457 | 0.0263 |
| 219907_at | NM_005653 | FRS3 | 10817 | 3.346 | 3.28 | 3.21 | 3.42 | 3.26 | 3.216 | 0.0034 |
| 219940_s_at | NM_018386 | PCID2 | 55795 | 7.335 | 7.33 | 7.4 | 7.23 | 7.43 | 7.433 | −0.018 |
| 219944_at | NM_024692 | CLIP4 | 79745 | 6.776 | 6.79 | 6.85 | 6.73 | 6.84 | 7.107 | 0.0046 |
| 220002_at | NM_018012 | KIF26B | 55083 | 3.065 | 3.04 | 3.12 | 3.2 | 2.95 | 2.969 | 0.1056 |
| 220007_at | NM_024770 | METTL8 | 79828 | 5.62 | 5.69 | 5.46 | 5.54 | 6.06 | 5.685 | −0.152 |
| 220020_at | NM_022098 | XPNPEP3 | 63929 | 4.445 | 4.54 | 4.37 | 4.59 | 4.42 | 4.597 | −0.01 |
| 220024_s_at | NM_020956 | PRX | 57716 | 3.151 | 3.31 | 3.38 | 3.26 | 3.29 | 3.066 | 0.089 |
| 220043_s_at | NM_005929 | MFI2 | 4241 | 2.958 | 2.82 | 3 | 3.36 | 3.05 | 3.011 | 0.2881 |
| 220046_s_at | NM_020307 | CCNL1 | 57018 | 7.805 | 7.95 | 7.77 | 7.66 | 7.99 | 7.787 | −0.164 |
| 220103_s_at | NM_016067 | MRPS18C | 51023 | 3.379 | 3.37 | 3.15 | 3.1 | 3.51 | 3.491 | −0.247 |
| 220114_s_at | NM_017564 | STAB2 | 55576 | 3.27 | 3.19 | 3.03 | 3.01 | 2.96 | 3.245 | −0.21 |
| 220166_at | NM_020348 | CNNM1 | 26507 | 3.084 | 3.12 | 3 | 3.08 | 2.92 | 3.036 | −0.058 |
| 220172_at | NM_025000 | C2orf37 | 80067 | 3.78 | 3.54 | 3.67 | 3.43 | 3.52 | 3.492 | −0.111 |
| 220208_at | NM_017587 | ADAWTS13 | 11093 | 3.553 | 3.45 | 3.38 | 3.24 | 3.22 | 3.559 | −0.189 |
| 220227_at | NM_024883 | CDH4 | 1002 | 4.057 | 3.95 | 4 | 3.74 | 4.08 | 4.039 | −0.136 |
| 220228_at | AB030653 | AP4E1 | 23431 | 2.586 | 2.61 | 2.84 | 2.74 | 2.8 | 2.742 | 0.1945 |
| 220229_s_at | NM_007347 | AP4E1 | 23431 | 3.224 | 3.37 | 3.33 | 2.93 | 3.39 | 3.451 | −0.169 |
| 220248_x_at | NM_018839 | NSFL1C | 55968 | 7.73 | 7.8 | 7.64 | 7.77 | 7.79 | 7.667 | −0.06 |
| 220253_s_at | NM_013437 | LRP12 | 29967 | 6.664 | 6.53 | 6.53 | 6.53 | 6.46 | 6.492 | −0.066 |
| 220254_at | NM_013437 | LRP12 | 29967 | 6.067 | 6.14 | 6.11 | 6 | 6.04 | 6.214 | −0.053 |
| 220271_x_at | NM_022785 | EFCAB6 | 64800 | 3.381 | 3.3 | 3.17 | 3.13 | 3.12 | 3.403 | −0.19 |
| 220312_at | NM_017708 | FAM83E | 54854 | 2.678 | 2.79 | 2.64 | 2.89 | 2.69 | 2.922 | 0.0289 |
| 220329_s_at | NM_017909 | RMND1 | 55005 | 7.25 | 7.4 | 7.39 | 7.41 | 7.14 | 7.385 | 0.0721 |
| 220349_s_at | NM_022759 | FLJ21865 | 64772 | 4.912 | 5.43 | 5.15 | 4.89 | 4.75 | 5.179 | −0.155 |
| 220395_at | NM_018602 | DNAJA4 | 55466 | 4.217 | 4.03 | 3.75 | 4.21 | 3.96 | 3.89 | −0.145 |
| 220434_at | NM_024876 | ADCK4 | 79934 | 2.907 | 3.05 | 2.99 | 3.13 | 2.89 | 3.289 | 0.0784 |
| 220439_at | NM_024892 | RIN3 | 79890 | 2.921 | 3.04 | 3.05 | 3.14 | 2.74 | 2.929 | 0.1119 |
| 220546_at | NM_024891 | MLL | 4297 | 3.088 | 3.12 | 3.05 | 3.14 | 3.1 | 3.172 | −0.011 |
| 220588_at | NM_017843 | BCAS4 | 55653 | 4.921 | 4.7 | 4.87 | 4.9 | 4.68 | 4.768 | 0.0761 |
| 220610_s_at | NM_006309 | LRRFIP2 | 9209 | 7.555 | 7.57 | 7.43 | 7.66 | 7.59 | 7.529 | −0.018 |
| 220688_s_at | NM_016183 | MRTO4 | 51154 | 8.371 | 8.4 | 8.17 | 8.3 | 8.15 | 8.282 | −0.149 |
| 220731_s_at | NM_018090 | NECAP2 | 55707 | 6.042 | 6.1 | 5.99 | 6.15 | 6.12 | 6.073 | 0.0025 |
| 220744_s_at | NM_018262 | IFT122 | 55764 | 4.261 | 4.64 | 4.44 | 4.61 | 4.67 | 4.779 | 0.0734 |
| 220801_s_at | NM_016527 | HAO2 | 51179 | 2.893 | 2.84 | 2.88 | 2.65 | 2.82 | 2.702 | −0.102 |
| 220947_s_at | NM_015527 | TBC1D10B | 26000 | 4.782 | 4.69 | 4.77 | 4.76 | 4.95 | 4.363 | 0.0311 |
| 220973_s_at | NM_030974 | SHARPIN | 81858 | 5.925 | 6.05 | 5.77 | 5.78 | 5.78 | 5.953 | −0.214 |
| 220986_s_at | NM_030953 | TIGD6 | 81789 | 3.129 | 3.08 | 3.03 | 3.23 | 3.27 | 2.984 | 0.0253 |
| 221037_s_at | NM_031291 | SLC2SA31 | 83447 | 2.641 | 2.49 | 2.58 | 2.54 | 2.44 | 2.505 | −0.005 |
| 221049_s_at | NM_013274 | POLL | 27343 | 4.716 | 4.99 | 4.68 | 4.91 | 4.76 | 5.176 | −0.054 |
| 221206_at | NM_024521 | PMS2 /// PMS2CL | 441194 /// 5395 | 5.744 | 5.64 | 5.64 | 5.88 | 5.83 | 5.936 | 0.0645 |
| 221211_s_at | NM_020152 | C21orf7 | 56911 | 3.777 | 3.6 | 3.86 | 3.8 | 3.53 | 3.877 | 0.1435 |
| 221290_s_at | NM_016473 | MUM1 | 84939 | 4.068 | 4.19 | 4.12 | 4.31 | 4.49 | 4.118 | 0.092 |
| 221307_at | NM_014592 | KCNIP1 | 30820 | 3.174 | 3.22 | 3.28 | 3.02 | 3.01 | 3.118 | −0.047 |
| 221335_x_at | NM_019108 | C19orf61 | 56006 | 4.607 | 4.42 | 4.66 | 4.52 | 4.77 | 4.6 | 0.0763 |
| 221438_s_at | NM_031275 | TEX12 | 56158 | 2.741 | 2.87 | 2.85 | 2.71 | 2.71 | 2.731 | −0.029 |
| 221455_s_at | NM_030753 | WNT3 | 7473 | 2.967 | 3.12 | 3.13 | 3.13 | 2.88 | 2.91 | 0.082 |
| 221499_s_at | AK_026970 | STX16 | 8675 | 7.435 | 7.37 | 7.34 | 7.54 | 7.36 | 7.486 | 0.0384 |
| 221500_s_at | BE782754 | STX16 | 8675 | 9.206 | 9.09 | 9.13 | 9.14 | 9.16 | 9.143 | −0.014 |
| 221534_at | AF073483 | C11orf68 | 83638 | 5.147 | 5.05 | 4.98 | 4.97 | 5.4 | 5.187 | −0.122 |
| 221571_at | AI721219 | TRAF3 | 7187 | 6.396 | 6.17 | 6.31 | 6.17 | 6.41 | 6.488 | −0.045 |
| 221614_s_at | BC005153 | RPH3AL | 9501 | 3.149 | 2.91 | 2.86 | 2.92 | 2.86 | 3.083 | −0.142 |
| 221619_s_at | AF189289 | MTCH1 | 23787 | 11.08 | 11.1 | 11.2 | 11 | 11.2 | 11.14 | 0.0289 |
| 221623_at | AF229053 | BCAN | 63827 | 2.712 | 2.86 | 2.64 | 2.63 | 2.62 | 2.575 | −0.152 |
| 221638_s_at | AF008937 | STX16 | 8675 | 5.122 | 5.34 | 5.18 | 4.92 | 5.28 | 5.187 | −0.185 |
| 221676_s_at | BC002342 | CORO1C | 23603 | 8.316 | 8.15 | 8.28 | 8.2 | 8.6 | 8.493 | 0.0091 |
| 221702_s_at | AF353992 | TM2D3 | 80213 | 7.986 | 7.99 | 8.03 | 7.89 | 7.98 | 7.904 | −0.026 |
| 221707_s_at | BC006116 | VPS53 | 55275 | 3.027 | 3.17 | 3.07 | 3.12 | 3.23 | 3.282 | −0.002 |
| 221809_at | AB040897 | RANBP10 | 57610 | 4 | 3.29 | 3.71 | 3.73 | 3.66 | 3.456 | 0.0693 |
| 221814_at | BF511315 | GPR124 | 25960 | 3.518 | 3.9 | 3.42 | 3.71 | 3.49 | 3.514 | −0.143 |
| 221845_s_at | AI655698 | CLPB | 81570 | 6.276 | 6.21 | 6.17 | 6.2 | 6.26 | 6.015 | −0.057 |
| 221854_at | AI378979 | PKP1 | 5317 | 7.218 | 7.2 | 7.24 | 7.19 | 6.99 | 6.994 | 0.0075 |
| 221865_at | BF969986 | C9orf91 | 203197 | 5.613 | 5.43 | 5.83 | 5.44 | 6.03 | 5.808 | 0.112 |
| 221870_at | AI417917 | EHD2 | 30846 | 6.152 | 6.08 | 6.1 | 6.26 | 6.46 | 6.321 | 0.0652 |
| 221881_s_at | AI638420 | CLIC4 | 25932 | 7.993 | 8.07 | 7.94 | 7.96 | 7.93 | 7.938 | −0.08 |
| 221891_x_at | AA704004 | HSPA8 | 3312 | 11.27 | 11.2 | 11.2 | 11.1 | 10.8 | 10.83 | −0.111 |
| 221897_at | AA205660 | TRIM52 | 84851 | 4.604 | 4.59 | 4.4 | 4.44 | 4.75 | 4.392 | −0.181 |
| 221899_at | AI809961 | N4BP2L2 | 10443 | 8.507 | 8.44 | 8.4 | 8.38 | 8.29 | 8.409 | −0.081 |
| 221920_s_at | BE677761 | SLC25A37 | 51312 | 5.146 | 5.6 | 5.14 | 5.6 | 4.99 | 5.281 | −0.003 |
| 221926_s_at | BF196320 | IL17RC | 84818 | 3.186 | 3.48 | 3.31 | 3.29 | 3.3 | 3.22 | −0.035 |
| 221960_s_at | AI89609 | RAB2A | 5862 | 6.285 | 6.09 | 6.35 | 6.42 | 6.38 | 6.389 | 0.2028 |
| 221990_at | AI948472 | PAX8 | 7849 | 2.747 | 2.65 | 2.78 | 2.71 | 2.57 | 2.772 | 0.0473 |
| 221998_s_at | BF062886 | VRK3 | 51231 | 7.06 | 7.02 | 7.18 | 7.1 | 7.22 | 7.168 | 0.0983 |
| 221999_at | BF062886 | VRK3 | 51231 | 4.506 | 4.72 | 4.84 | 4.71 | 4.87 | 4.803 | 0.161 |
| 222010_at | BF224073 | TCP1 | 6950 | 7.422 | 7.2 | 7.27 | 7.24 | 7.35 | 7.369 | −0.057 |
| 222011_s_at | BF224073 | TCP1 | 6950 | 6.923 | 6.62 | 6.95 | 6.74 | 6.64 | 6.838 | 0.0744 |
| 222035_s_at | AI984479 | PAPOLA | 10914 | 9.502 | 9.61 | 9.45 | 9.55 | 9.61 | 9.632 | −0.053 |
| 222043_at | AI982754 | CLU | 1191 | 2.887 | 2.94 | 3.04 | 2.78 | 2.98 | 2.817 | −0.005 |
| 222154_s_at | AK002064 | LOC26010 | 26010 | 8.547 | 8.35 | 8.53 | 8.51 | 8.73 | 8.619 | 0.073 |
| 222169_x_at | N71739 | SH2D3A | 10045 | 3.599 | 3.95 | 3.5 | 3.63 | 3.72 | 3.91 | −0.207 |
| 222176_at | AK021487 | PTEN | 5728 | 3.277 | 3.07 | 2.94 | 3.22 | 3.18 | 3.007 | −0.096 |
| 222188_at | AK023069 | C9orf156 | 51531 | 2.902 | 2.8 | 2.78 | 2.86 | 2.59 | 2.893 | −0.033 |
| 222195_s_at | AK023069 | C9orf156 | 51531 | 5.353 | 5.33 | 5.24 | 5.58 | 5.4 | 5.425 | 0.072 |
| 222220_s_at | AK027245 | TSNAXIP1 | 55815 | 3.118 | 3.27 | 3.19 | 3.05 | 3.24 | 3.235 | −0.074 |
| 222231_s_at | AK025328 | LRRCS9 | 55379 | 10.33 | 10.2 | 10.4 | 10.2 | 9.97 | 9.993 | 0.0271 |
| 222255_at | AB046840 | PRX | 57716 | 2.538 | 2.44 | 2.58 | 2.53 | 2.49 | 2.604 | 0.0658 |
| 222305_at | AW975638 | HK2 | 3099 | 5.033 | 5.17 | 5.12 | 5.12 | 5.09 | 5.439 | 0.0217 |
| 222346_at | AI633741 | LAMA1 | 284217 | 3.445 | 3.44 | 3.37 | 3.41 | 3.41 | 3.341 | −0.054 |
| 222348_at | AW971134 | MAST4 | 375449 | 4.303 | 4.33 | 4.22 | 4.1 | 4.15 | 4.353 | −0.157 |
| 222353_at | AV720842 | LIMD1 | 8994 | 2.804 | 3.06 | 3.19 | 3.12 | 3 | 3.145 | 0.2193 |
| 222383_s_at | AW003512 | ALOXE3 | 59344 | 4.179 | 4.03 | 4.2 | 4.21 | 4.88 | 4.489 | 0.1007 |
| 31846_at | AW003733 | RHOD | 29984 | 8.085 | 8.1 | 8.08 | 8.14 | 8.18 | 8.224 | 0.0207 |
| 31861_at | L14754 | IGHMBP2 | 3508 | 5.492 | 5.28 | 5.4 | 5.17 | 5.18 | 5.277 | −0.1 |
| 32094_at | AB017915 | CHST3 | 9469 | 4.033 | 4.12 | 4.17 | 4.03 | 4.32 | 4.089 | 0.0221 |
| 33132_at | U37012 | CPSF1 | 29894 | 5.606 | 5.65 | 5.72 | 5.67 | 5.64 | 5.739 | 0.0674 |
| 34478_at | X79780 | RAB11B | 9230 | 3.143 | 3.08 | 3.32 | 3 | 3.38 | 3.183 | 0.0462 |
| 36865_at | AB018302 | ANGEL1 | 23357 | 4.5 | 4.47 | 4.38 | 4.5 | 4.59 | 4.514 | −0.04 |
| 37005_at | D28124 | NBL1 | 4681 | 7.09 | 7.09 | 7 | 6.97 | 7.23 | 7.101 | −0.102 |
| 37566_at | AB028968 | KIAA1045 | 23349 | 2.74 | 2.71 | 2.9 | 2.71 | 2.81 | 2.662 | 0.0834 |
| 37860_at | AL049942 | ZNF337 | 26152 | 5.892 | 5.56 | 5.71 | 5.74 | 5.65 | 5.695 | 0.0023 |
| 37872_at | AF072468 | JRK | 8629 | 4.324 | 4.14 | 4.27 | 4.05 | 4.22 | 4.389 | −0.07 |
| 38269_at | AL050147 | PRKD2 | 25865 | 5.955 | 6.1 | 6.29 | 6.06 | 6.21 | 6.047 | 0.1467 |
| 38447_at | U08438 | ADRBK1 | 156 | 4.316 | 4.26 | 4.39 | 4.14 | 4.41 | 4.26 | −0.026 |
| 38918_at | AF083105 | SOX13 | 9580 | 3.721 | 3.88 | 3.79 | 4.04 | 3.92 | 3.904 | 0.1153 |
| 39817_s_at | AF040105 | C6orf108 | 10591 | 6.844 | 6.85 | 6.86 | 6.95 | 7.08 | 6.947 | 0.058 |
| 40148_at | U62325 | APBB2 | 323 | 5.528 | 5.47 | 5.42 | 5.55 | 5.71 | 5.433 | −0.01 |
| 40273_at | AA485440 | SPHK2 | 56848 | 4.562 | 4.43 | 4.51 | 4.57 | 4.8 | 4.647 | 0.0466 |
| 41220_at | AB023208 | 10-Sep | 10801 | 10.48 | 10.4 | 10.5 | 10.6 | 106 | 10.46 | 0.1312 |
| 41657_at | AF035625 | STK11 | 6794 | 3.789 | 3.93 | 3.91 | 3.73 | 3.97 | 3.926 | −0.035 |
| 41660_at | AL031588 | CELSR1 | 9620 | 5.704 | 5.74 | 5.79 | 5.76 | 5.77 | 5.558 | 0.0546 |
| 44696_at | AA915989 | TBC1D13 | 54662 | 5.513 | 5.43 | 5.38 | 5.49 | 5.48 | 5.463 | −0.035 |
| 45297_at | AI417917 | EHD2 | 30846 | 5.607 | 5.6 | 5.7 | 5.61 | 5.78 | 5.502 | 0.0502 |
| 47530_at | AA748492 | C9orf156 | 51531 | 5.211 | 5.12 | 5.19 | 5.07 | 5.27 | 5.137 | −0.036 |
| 53987_at | AL041852 | RANBP10 | 57610 | 4.188 | 4.06 | 4.09 | 4.01 | 4.2 | 3.951 | −0.072 |
| 54037_at | AL041451 | HPS4 | 89781 | 4.368 | 4.08 | 4.11 | 4.35 | 4.36 | 4.285 | 0.0032 |
| 60471_at | AA625133 | RIN3 | 79890 | 4.074 | 4.22 | 4.44 | 4.36 | 4.33 | 4.366 | 0.2497 |
| 64440_at | AI560217 | IL17RC | 84818 | 4.138 | 4.02 | 4.18 | 4 | 3.89 | 4.24 | 0.0082 |
| 65493_at | AA555088 | HEATR6 | 63897 | 6.182 | 6.2 | 6.06 | 6.11 | 6.17 | 6.139 | −0.105 |
| 65635_at | AL044097 | FLJ21865 | 64772 | 5.008 | 4.97 | 5 | 4.79 | 5.05 | 4.97 | −0.094 |
| 65718_at | AI655903 | GPR124 | 25960 | 3.116 | 3.41 | 3.17 | 3.47 | 3.35 | 3.362 | 0.0559 |
| 91920_at | AI205180 | BCAN | 63827 | 3.243 | 3.59 | 3.34 | 3.24 | 3.32 | 3.297 | −0.127 |

| | | | | | | | | | | | BPLER |
|---|---|---|---|---|---|---|---|---|---|---|---|
| | | | | HMLER | | | | | | | (GFP |
| | Representative | | | (hA6 vs | | | | | | | vs |
| Probe Set ID | Public ID | Gene Symbol | Entrez Gene | SCR) | SCR_BPLER_A | SCR_BPLER_B | GFP_BPLER_A | GFP_BPLER_B | ha6_BPLER_A | ha6_BPLER_B | SCR) |
| 117_at | X51757 | HSPA6 | 3310 | −0.2317 | 3.056 | 2.91 | 2.921 | 2.95 | 2.97 | 3.013 | −0.05 |
| 121_at | X69699 | RAX8 | 7849 | −0.1178 | 4.867 | 4.96 | 4.875 | 4.98 | 4.93 | 5.042 | 0.015 |
| 1487_at | L38487 | ESRRA | 2101 | 0.0179 | 5.61 | 5.53 | 5.343 | 5.69 | 5.63 | 5.742 | −0.055 |
| 200002_at | NM_007209 | RPL35 | 11224 | −0.063 | 11.82 | 11.8 | 11.87 | 11.7 | 11.7 | 11.68 | −0.012 |
| 200017_at | NM_002954 | RPS27A /// UBB /// | 6233 /// 7314 /// | −0.0664 | 12.65 | 12.6 | 12.65 | 12.6 | 12.5 | 12.48 | −0.012 |
| | | UBC | 7316 | | | | | | | | |
| 200019_s_at | NM_001997 | FAU | 2197 | 0.0174 | 11.96 | 12 | 12 | 12 | 12 | 11.92 | −0.009 |
| 200022_at | NM_000979 | RPL18 | 6141 | −0.1165 | 12.74 | 12.8 | 12.71 | 12.7 | 12.6 | 12.57 | −0.06 |
| 200024_at | NM_001009 | RPSS | 6193 | −0.0526 | 12.38 | 12.4 | 12.42 | 12.3 | 12.2 | 12.4 | −0.035 |
| 200037_s_at | NM_016587 | CBX3 /// LOC653972 | 11335 /// 653972 | −0.5741 | 10.66 | 10.6 | 10.51 | 10.6 | 9.8 | 9.764 | −0.08 |
| 200049_at | NM_007067 | MYST2 | 11143 | −0.28 | 6.527 | 6.69 | 6.678 | 6.54 | 6.58 | 6.513 | −0.002 |
| 200064_at | AF275719 | HSP90AB1 | 3326 | −0.3251 | 11.03 | 11 | 10.94 | 10.9 | 10.3 | 10.38 | −0.091 |
| 200067_x_at | AL078595 | SNX3 | 8724 | 0.0456 | 10.76 | 10.8 | 10.77 | 10.9 | 10.8 | 10.7 | 0.036 |
| 200601_at | U48734 | ACTN4 | 81 | 0.0385 | 8.422 | 8.35 | 8.314 | 8.12 | 8.93 | 8.997 | −0.166 |
| 200602_at | NM_000484 | APP | 351 | −0.111 | 10.18 | 10.1 | 10 | 10 | 9.98 | 9.857 | −0.157 |
| 200618_at | NM_006148 | LASP1 | 3927 | −0.0405 | 8.398 | 8.38 | 8.37 | 8.44 | 7.98 | 8.04 | 0.016 |
| 200622_x_at | AV685208 | CALM1 /// C4LM2 /// | 801 /// 805 /// 808 | 0.0402 | 6.068 | 6.47 | 5.977 | 6.26 | 6.71 | 6.699 | −0.15 |
| | | CALM3 | | | | | | | | | |
| 200623_s_at | NM_005184 | CALM1 /// CALM2 /// | 801 /// 805 /// 808 | 0.353 | 5.322 | 5.58 | 5.394 | 5.55 | 5.5 | 5.366 | 0.021 |
| | | CALM3 | | | | | | | | | |
| 200627_at | BC003005 | PTGES3 | 10728 | −0.0974 | 11.04 | 11 | 11.15 | 11 | 10.9 | 10.96 | 0.048 |
| 200632_s_at | NM_006096 | NDRG1 | 10397 | −0.273 | 8.914 | 9.11 | 8.928 | 9.13 | 8.44 | 8.268 | 0.015 |
| 200633_at | NM_018955 | RPS27A /// UBB /// | 6233 /// 7314 /// | 0.0568 | 12.59 | 12.6 | 12.6 | 12.6 | 12.2 | 12.27 | −0.012 |
| | | UBC | 7316 | | | | | | | | |
| 200653_s_at | M27319 | CALM1 /// CALM2 /// | 801 /// 805 /// 808 | −0.129 | 8.962 | 8.95 | 9.001 | 9.1 | 8.8 | 8.861 | −9.09 |
| | | CALM3 | | | | | | | | | |
| 200655_s_at | NM_006888 | CALM1 /// CALM2 /// | 801 /// 805 /// 808 | 0.0876 | 9.005 | 8.96 | 8.969 | 9.09 | 9.04 | 8.996 | 0.048 |
| | | CALM3 | | | | | | | | | |
| 200664_s_at | BG537255 | DNAJB1 | 3337 | −0.1659 | 7.581 | 7.57 | 7.669 | 7.83 | 7.36 | 7.289 | 0.177 |
| 200666_s_at | NM_006145 | DNAJB1 | 3337 | −0.0689 | 8.213 | 8.08 | 8.104 | 8.22 | 7.87 | 7.773 | 0.015 |
| 200667_at | BF448062 | UBE2D3 | 7323 | −0.0772 | 9.646 | 9.69 | 9.629 | 9.59 | 9.65 | 9.49 | −0.06 |
| 200668_s_at | BC003395 | UBE2D3 | 7323 | −0.0717 | 10.26 | 10.2 | 10.23 | 10.3 | 10.2 | 10.28 | 0.015 |
| 200669_s_at | NM_003340 | UBE2D3 | 7323 | 0.0298 | 9.072 | 9.14 | 9.089 | 9.24 | 9.21 | 9.251 | 0.057 |
| 200687_s_at | NM_012426 | SF3B3 | 23450 | 0.0056 | 6.967 | 7.04 | 7.014 | 7 | 6.76 | 6.915 | 0.002 |
| 200688_at | D13642 | SF3B3 | 23450 | −0.0917 | 3.564 | 3.4 | 3.334 | 3.29 | 3.44 | 3.544 | −0.17 |
| 200689_x_at | NM_001404 | EEF1G | 1937 | −0.1216 | 12.38 | 12.4 | 12.34 | 12.3 | 12.3 | 12.27 | −0.056 |
| 200696_s_at | NM_000177 | GSN | 2934 | 0.1375 | 7.573 | 7.74 | 7.525 | 7.66 | 7.28 | 7.276 | −0.063 |
| 200707_at | NM_002743 | PRXCSH | 5589 | −0.0502 | 6.921 | 5.92 | 7.032 | 6.96 | 6.76 | 6.73 | 0.074 |
| 200737_at | NM_000791 | PGK1 | 5230 | −0.347 | 8.105 | 8.24 | 8.05 | 8.34 | 7.78 | 7.827 | 0.024 |
| 200738_s_at | NM_000291 | PGK1 | 5230 | −0.0429 | 10.71 | 10.8 | 10.72 | 10.9 | 10.6 | 10.68 | 0.036 |
| 200753_x_at | BE866585 | SFRS2 | 6427 | −0.2676 | 8.266 | 8.23 | 8.155 | 8.05 | 8.28 | 8.275 | −0.142 |
| 200754_x_at | NM_003016 | SF952 | 6427 | 0.2013 | 10.26 | 10.1 | 10.25 | 10.1 | 10.3 | 10.36 | −0.016 |
| 200768_s_at | BC001686 | MAT2A | 4144 | −0.0171 | 9.078 | 8.92 | 8.907 | 8.9 | 9.01 | 8.964 | −0.096 |
| 200769_s_at | NM_005911 | MAT2A | 4144 | −0.251 | 5.042 | 5.16 | 5.331 | 5.16 | 5.35 | 5.1 | 0.148 |
| 200806_s_at | BE256479 | HSPD1 | 3329 | −0.0658 | 11.95 | 12 | 12.06 | 12.1 | 11.9 | 11.93 | 0.052 |
| 200807_s_at | NM_002156 | HSPD1 | 3329 | 0.0563 | 12.14 | 12.1 | 12.15 | 12.2 | 12.2 | 12.19 | 0.02 |
| 200812_at | NM_006429 | CCT7 | 10574 | 0.0627 | 9.078 | 9.06 | 9.045 | 9.09 | 9.25 | 9.226 | −0.004 |
| 200823_x_at | NM_000992 | LOC100131713 /// | 100131713 /// | −0.3518 | 12.32 | 12.4 | 12.27 | 12.2 | 12.1 | 12.19 | −0.083 |
| | | RPL29 /// RPL29P4 | 387101 /// 6159 | | | | | | | | |
| 200828_s_at | BE871379 | ZNF207 | 7756 | −0.1068 | 9.835 | 9.82 | 9.854 | 9.78 | 9.61 | 9.867 | −0.011 |
| 200829_x_at | NM_003457 | ZNF207 | 7756 | −0.0164 | 9.83 | 9.79 | 9.826 | 9.77 | 9.84 | 9.701 | −0.012 |
| 200847_s_at | NM_016127 | TMEM66 | 51669 | −0.352 | 11.32 | 11.3 | 11.25 | 11.3 | 10.5 | 10.51 | −0.035 |
| 200854_at | AB028970 | NCOR1 | 9611 | 0.1823 | 6.772 | 6.67 | 6.682 | 6.6 | 6.95 | 7.186 | −0.081 |
| 200857_s_at | NM_006311 | NCOR1 | 9611 | 0.2248 | 6.646 | 6.64 | 6.864 | 6.53 | 6.9 | 7 | 0.056 |
| 200873_s_at | NM_006585 | CCT8 | 10694 | 0.0317 | 10.98 | 11.1 | 10.87 | 10.9 | 10.9 | 10.84 | −0.127 |
| 200877_at | NM_006430 | CCT4 | 10575 | −0.066 | 11.29 | 11.3 | 11.29 | 11.3 | 11 | 10.99 | 0.007 |
| 200880_at | AL534104 | DNAJA1 | 3301 | −0.0998 | 8.73 | 8.72 | 8.683 | 8.59 | 8.32 | 8.429 | −0.089 |
| 200881_s_at | NM_001539 | DNAJA1 | 3301 | −0.3179 | 10.03 | 10 | 9.963 | 9.89 | 9.35 | 9.436 | −0.11 |
| 200892_s_at | BC000451 | SFRS10 | 6434 | −0.0131 | 8.541 | 8.58 | 8.47 | 8.53 | 8.77 | 8.723 | −0.064 |
| 200893_at | NM_004593 | SFRS10 | 6434 | 0.1469 | 10.91 | 10.9 | 10.91 | 10.9 | 11.1 | 11.09 | 0.025 |
| 200894_s_at | AA894574 | FKBP4 | 2288 | −0.4274 | 6.397 | 6.48 | 6.212 | 6.49 | 6.28 | 6.451 | −0.088 |
| 290895_s_at | NM_002014 | FXBP4 | 2288 | −0.1886 | 7.055 | 7.01 | 7.009 | 7.15 | 7.2 | 7.28 | 0.049 |
| 200896_x_at | NM_004494 | HDGF | 3068 | −0.1104 | 9.884 | 9.9 | 9.832 | 9.95 | 9.87 | 9.884 | −0.002 |
| 200910_at | NM_005998 | CCT3 | 7203 | −0.16 | 9.888 | 9.93 | 9.837 | 9.79 | 9.52 | 9.606 | −0.095 |
| 200912_s_at | NM_001967 | EIF4A2 | 1974 | −0.1081 | 12.03 | 12.1 | 12.02 | 12 | 11.8 | 11.78 | −0.044 |
| 200936_at | NM_000973 | RPL8 | 6132 | −0.027 | 12.93 | 12.9 | 12.94 | 13 | 12.9 | 12.91 | 0.035 |
| 200965_s_at | NM_006720 | ABLIM1 | 3983 | 0.2212 | 7.503 | 7.67 | 7.902 | 7.84 | 8.03 | 7.963 | 0.288 |
| 200983_x_at | BF983379 | CD59 | 966 | −0.2671 | 9.372 | 9.44 | 9.219 | 9.4 | 9.04 | 9.159 | −0.097 |
| 200984_s_at | X16447 | CD59 | 966 | −0.3578 | 8.573 | 8.65 | 8.551 | 8.53 | 8.17 | 8.191 | −0.068 |
| 200985_s_at | NM_000611 | CD59 | 966 | −0.0718 | 8.865 | 8.77 | 8.918 | 8.85 | 8.6 | 8.597 | 0.066 |
| 201023_at | NM_005642 | TAF7 | 6879 | 0.1382 | 7.934 | 8.06 | 8.036 | 7.97 | 8.56 | 8.428 | 0.007 |
| 201066_at | NM_001916 | CYC1 | 1537 | 0.2258 | 8.599 | 8.68 | 8.703 | 8.81 | 9.17 | 9.186 | 0.115 |
| 201079_at | NM_004710 | SYNGR2 | 9144 | −0.0822 | 7.344 | 7.42 | 7.368 | 7.21 | 7.32 | 7.544 | −0.092 |
| 201091_s_at | BE748755 | CBX3 /// LOC653972 | 11335 /// 653972 | −0.2783 | 9.562 | 9.57 | 9.458 | 9.44 | 9.28 | 9.161 | −0.114 |
| 201129_at | NM_006276 | SFRS7 | 6432 | 0.1669 | 8.639 | 8.65 | 8.525 | 8.6 | 8.92 | 9.033 | −0.085 |
| 201132_at | NM_019597 | HNRNPH2 | 3188 | −0.3154 | 7.804 | 7.85 | 7.803 | 7.91 | 7.39 | 7.507 | 0.029 |
| 201140_s_at | NM_004583 | RAB5C | 5878 | −0.2665 | 7.394 | 7.53 | 7.502 | 7.54 | 6.83 | 7.012 | 0.06 |
| 201156_s_at | AF141304 | RAB5C | 5878 | −0.3782 | 7.33 | 7.47 | 7.46 | 7.45 | 6.91 | 6.918 | 0.057 |
| 201162_at | NM_001553 | IGFBP7 | 3490 | −0.452 | 10.35 | 10.3 | 10.17 | 10 | 9.83 | 9.831 | −0.219 |
| 201163_s_at | NM_001553 | IGFBP7 | 3490 | −0.5632 | 11.23 | 11.2 | 11.04 | 11.1 | 10.9 | 10.77 | −0.113 |
| 201173_x_at | NM_006600 | NUDC | 10726 | 0.0768 | 7.482 | 7.59 | 7.6 | 7.47 | 7.93 | 8.066 | −1E−06 |
| 201182_s_at | AI761771 | CHD4 | 1108 | 0.1079 | 6.383 | 6.42 | 6.352 | 6.55 | 6.49 | 6.355 | 0.049 |
| 201183_s_at | AI613273 | CHD4 | 1108 | −0.0307 | 7.253 | 7.28 | 7.293 | 7.27 | 7.19 | 7.287 | 0.015 |
| 201184_s_at | NM_001273 | CHD4 | 1108 | −0.2092 | 6.887 | 6.95 | 6.957 | 6.87 | 6.87 | 6.888 | −0.008 |
| 201194_at | NM_003009 | SEPW1 | 6415 | 0.0624 | 9.667 | 9.79 | 9.678 | 9.74 | 9.43 | 9.572 | −0.02 |
| 201218_at | N23018 | CTBP2 | 1488 | −0.2923 | 9.926 | 9.87 | 9.81 | 9.79 | 9.47 | 9.408 | −0.099 |
| 201219_at | AW269836 | CTBP2 | 1488 | −0.0279 | 8.095 | 8.03 | 7.907 | 7.94 | 7.91 | 7.864 | −0.139 |
| 201220_x_at | NM_001329 | CTBP2 | 1488 | −0.0098 | 10.38 | 10.4 | 10.38 | 10.2 | 10.3 | 10.32 | −0.067 |
| 201249_at | AI091047 | SLC2A1 | 6513 | −0.2884 | 4.651 | 4.9 | 4.667 | 4.69 | 4.8 | 4.655 | −0.1 |
| 201250_s_at | NM_006516 | SLC2A1 | 6513 | 0.1382 | 8.222 | 8.38 | 8.393 | 8.4 | 8.35 | 8.296 | 0.095 |
| 201269_s_at | AB028991 | NUDCD3 | 23386 | −0.1358 | 3.412 | 3.52 | 3.091 | 2.97 | 3.13 | 3.488 | −0.435 |
| 201270_x_at | NM_015332 | NUDCD3 | 23386 | 0.0776 | 7.723 | 7.68 | 7.744 | 7.7 | 7.46 | 7.428 | 0.022 |
| 201301_s_at | BC000182 | ANXA4 | 307 | −0.0716 | 9.693 | 9.71 | 9.604 | 9.71 | 9.33 | 9.378 | −0.044 |
| 201302_at | NM_001153 | ANXA4 | 307 | −0.2302 | 9.168 | 9.19 | 9.106 | 9.2 | 8.36 | 8.445 | −0.024 |
| 201326_at | BE737030 | CCT6A | 908 | 0.0292 | 9.957 | 9.93 | 9.969 | 9.93 | 10.1 | 10.16 | 0.005 |
| 201327_s_at | NM_001762 | CCT6A | 908 | −0.1369 | 10.72 | 10.7 | 10.69 | 10.7 | 10.7 | 10.65 | −0.033 |
| 201331_s_at | BC004973 | STAT6 | 6778 | −0.1349 | 6.527 | 6.53 | 6.201 | 6.6 | 6.56 | 6.689 | −0.123 |
| 201332_s_at | NM_003153 | STAT6 | 6778 | 0.0595 | 3.415 | 3.54 | 3.501 | 3.55 | 3.55 | 3.469 | 0.046 |
| 201373_at | NM_000445 | PLEC1 | 5339 | 0.0761 | 7.3 | 7.17 | 7.243 | 7.42 | 7.54 | 7.527 | 0.103 |
| 201379_s_at | NM_003288 | TPD52L2 | 7165 | −0.1087 | 7.941 | 8.09 | 8.025 | 8.04 | 7.82 | 7.848 | 0.015 |
| 201381_x_at | AF057356 | CACYBP | 27101 | −0.1076 | 9.433 | 9.59 | 9.43 | 9.48 | 9.91 | 9.138 | −0.056 |
| 201382_at | NM_014412 | CACYBP | 27101 | −0.002 | 3.524 | 3.49 | 3.564 | 3.44 | 3.25 | 3.305 | −1E−03 |
| 201388_at | NM_002809 | PSMD3 | 5709 | −0.0469 | 6.998 | 6.92 | 7.039 | 6.97 | 7.12 | 7.11 | 0.041 |
| 201400_at | NM_002795 | PSMB3 | 5691 | −0.0568 | 9.559 | 9.69 | 9.653 | 9.7 | 9.64 | 9.863 | 0.051 |
| 201401_s_at | M80776 | ADRBK1 | 156 | −0.2091 | 3.662 | 3.66 | 3.929 | 3.62 | 3.62 | 3.709 | 0.109 |
| 201402_at | NM_001619 | ADRBK1 | 156 | −0.1521 | 4.319 | 4.09 | 4.079 | 4.02 | 4.04 | 3.857 | −0.156 |
| 201423_s_at | AL037208 | CUL4A | 8451 | 0.0538 | 6.074 | 6 | 6.207 | 6.14 | 6.31 | 6.253 | 0.137 |
| 201424_s_at | NM_003589 | CUL4A | 8451 | 0.0577 | 7.187 | 7.05 | 6.966 | 7.02 | 7.23 | 7.086 | −0.127 |
| 201491_at | NM_012111 | AHSA1 | 10598 | −0.1327 | 8.68 | 8.75 | 8.811 | 8.71 | 8.71 | 8.79 | 0.146 |
| 201559_s_at | AF109196 | CLIC4 | 25932 | 0.4102 | 6.52 | 6.53 | 6.421 | 6.44 | 6.33 | 6.331 | −0.096 |
| 201560_at | NM_013943 | CLIC4 | 25932 | −0.0537 | 9.339 | 9.32 | 9.322 | 9.32 | 9.55 | 9.445 | −0.004 |
| 201564_s_at | NM_003088 | FSCN1 | 6624 | 0.3347 | 5.8 | 5.63 | 5.995 | 6.15 | 6.06 | 6.035 | 0.357 |
| 201578_at | NM_005397 | PODXL | 5420 | 0.1395 | 4.111 | 4.31 | 4.32 | 4.06 | 4.52 | 4.938 | −0.021 |
| 201605_x_at | NM_004368 | CNN2 | 1265 | 0.0342 | 6.337 | 6.12 | 6.08 | 5.95 | 5.97 | 5.891 | −0.214 |
| 201621_at | NM_005380 | NBL1 | 4681 | 0.0744 | 5.111 | 5.29 | 5.264 | 5.31 | 5.15 | 4.887 | 0.091 |
| 201623_s_at | BC000629 | DARS | 1615 | 0.0646 | 10.17 | 10.3 | 10.21 | 10.3 | 10 | 10.2 | 0.037 |
| 201624_at | NM_001349 | DARS | 1615 | 0.1995 | 7.41 | 7.54 | 7.256 | 7.53 | 7.54 | 7.209 | −0.082 |
| 201635_s_at | AI990766 | FXR1 | 8087 | −0.6508 | 8.974 | 8.86 | 8.918 | 8.82 | 8.1 | 8.117 | −0.046 |
| 201636_at | BG025078 | FXR1 | 8087 | −0.0022 | 8.199 | 8.2 | 8.103 | 8.17 | 7.58 | 7.521 | −0.067 |
| 201637_s_at | NM_005087 | FXR1 | 8087 | −0.1972 | 9.866 | 9.9 | 9.802 | 9.82 | 9.31 | 9.305 | −0.072 |
| 201638_s_at | BE676642 | CPSF1 | 29894 | 0.0567 | 3.202 | 3.48 | 3.013 | 3.07 | 3.16 | 3.149 | −0.301 |
| 201639_s_at | NM_013291 | CPSF1 | 29894 | 0.0833 | 6.637 | 6.72 | 6.777 | 6.61 | 7.05 | 7.192 | 0.015 |
| 201642_at | NM_005534 | IFNGR2 | 3460 | −0.1021 | 7.115 | 7.21 | 7.11 | 7.31 | 7.13 | 7.109 | 0.045 |
| 201643_x_at | NM_016604 | JMJD1B | 51780 | 0.151 | 6.293 | 6.19 | 6.064 | 6.34 | 6.13 | 6.055 | −0.035 |
| 201654_s_at | AI991033 | HSPG2 | 3339 | −0.0376 | 2.874 | 2.98 | 3.022 | 2.74 | 2.88 | 2.867 | −0.042 |
| 201655_s_at | M85289 | HSPG2 | 3339 | 0.1889 | 5.268 | 5.58 | 5.862 | 5.93 | 5.71 | 5.215 | 0.475 |
| 201688_s_at | BG389015 | TPD52 | 7163 | −0.0706 | 6.998 | 7.23 | 7.175 | 7.06 | 7.66 | 7.795 | 0.003 |
| 201689_s_at | BE974098 | TPD52 | 7163 | −0.1454 | 7.713 | 7.57 | 7.56 | 7.67 | 8.27 | 8.339 | −0.03 |
| 201690_s_at | AA524023 | TPD52 | 7163 | 0.0707 | 8.797 | 8.78 | 8.905 | 8.86 | 9.63 | 9.698 | 0.094 |
| 201691_s_at | NM_005079 | TPD52 | 7163 | 0.0164 | 3.148 | 3.33 | 3.225 | 3.16 | 3.24 | 3.341 | −0.044 |
| 201711_x_at | AI681120 | RANBP2 | 5903 | −0.1233 | 7.763 | 7.66 | 7.695 | 7.75 | 7.42 | 7.663 | 0.007 |
| 201712_s_at | NM_006267 | RANBP2 | 5903 | 0.0437 | 6.595 | 6.29 | 6.329 | 6.2 | 6.59 | 6.906 | −0.181 |
| 201713_s_at | D42063 | RANBP2 | 5903 | −0.1113 | 7.707 | 7.71 | 7.73 | 7.61 | 7.64 | 7.564 | −0.036 |
| 201717_at | NM_004927 | MRPL49 | 740 | 0.1026 | 7.917 | 7.95 | 8.053 | 8 | 8.3 | 8.321 | 0.094 |
| 201751_at | NM_014876 | JOSD1 | 9929 | −0.1277 | 7.101 | 7.06 | 7.341 | 7.02 | 7.49 | 7.663 | 0.099 |
| 201772_at | NM_015878 | AZIN1 | 51582 | 0.1604 | 8.618 | 8.68 | 8.43 | 8.37 | 8.72 | 8.687 | −0.247 |
| 201841_s_at | NM_001540 | HSPB1 | 3315 | −0.1069 | 11.41 | 11.4 | 11.43 | 11.3 | 11.3 | 11.06 | 0.013 |
| 201842_s_at | AI826799 | EFEMP1 | 2202 | −0.0396 | 10.97 | 10.9 | 10.8 | 10.8 | 10.6 | 10.54 | −0.139 |
| 201843_s_at | NM_004105 | EFEMP1 | 2202 | −0.2745 | 9.291 | 9.24 | 9.132 | 9.01 | 8.55 | 8.365 | −0.194 |
| 201853_s_at | NM_021873 | CDC258 | 994 | −0.0122 | 8.858 | 8.99 | 9.003 | 8.88 | 8.83 | 8.757 | 0.017 |
| 201913_s_at | NM_025233 | COASY | 80347 | 0.0217 | 7.298 | 7.41 | 7.423 | 7.52 | 7.65 | 7.531 | 0.12 |
| 201922_at | NM_014886 | TINP1 | 10412 | 0.1132 | 11.47 | 11.4 | 11.47 | 11.4 | 11.3 | 11.28 | −0.006 |
| 201971_s_at | NM_001690 | ATP6V1A | 523 | −0.2684 | 5.347 | 5.42 | 5.43 | 5.58 | 4.67 | 4.552 | 0.121 |
| 201972_at | AF113129 | ATP6V1A | 523 | −0.0098 | 9.308 | 9.32 | 9.197 | 9.38 | 9.02 | 8.968 | −0.023 |
| 201983_s_at | AW157070 | EGFR | 1956 | 0.0515 | 10.54 | 10.5 | 10.47 | 10.5 | 10.1 | 10.07 | −0.015 |
| 201984_s_at | NM_005228 | EGFR | 1956 | 0.0669 | 7.609 | 7.59 | 7.694 | 7.53 | 7.21 | 7.204 | 0.016 |
| 201994_at | NM_012286 | MORF4L2 | 9643 | −0.1301 | 10.97 | 11 | 10.88 | 11 | 10.9 | 10.92 | −0.061 |
| 202043_s_at | NM_004595 | SMS | 6611 | −0.1768 | 7.785 | 7.88 | 7.55 | 7.67 | 8.13 | 7.926 | −0.223 |
| 202055_at | AA652173 | KPNA1 | 3836 | 0.0678 | 7.489 | 7.31 | 7.264 | 7.32 | 7.5 | 7.417 | −0.11 |
| 202056_at | AW051311 | KPNA1 | 3836 | 0.2267 | 6.851 | 6.86 | 6.835 | 6.88 | 7.06 | 7 | −0.002 |
| 202057_at | BC002374 | KPNA1 | 3836 | −0.0518 | 5.676 | 5.69 | 5.597 | 5.58 | 5.53 | 5.5 | −0.096 |
| 202058_s_at | BC002374 | KPNA1 | 3836 | 0.0557 | 6.602 | 6.54 | 6.449 | 6.4 | 6.43 | 6.467 | −0.148 |
| 202059_s_at | NM_002264 | KPNA1 | 3836 | 0.0323 | 7.478 | 7.32 | 7.179 | 7.44 | 7.64 | 7.641 | −0.093 |
| 202067_s_at | AI861942 | LDLR | 3949 | 0.0131 | 6.003 | 6.17 | 6.282 | 6.1 | 6.54 | 6.758 | 0.107 |
| 202068_s_at | NM_000527 | LDLR | 3949 | 0.012 | 7.912 | 7.93 | 7.844 | 8.13 | 8.68 | 8.775 | 0.07 |
| 202104_s_at | NM_003319 | SPG7 | 6687 | −0.1644 | 6.354 | 6.46 | 6.597 | 6.44 | 6.3 | 6.147 | 0.113 |
| 202106_at | NM_005895 | GOLGA3 | 2802 | 0.068 | 5.937 | 6.11 | 6.132 | 5.97 | 6.45 | 6.383 | 0.028 |
| 202151_s_at | NM_016172 | UBAC1 | 10422 | 0.0168 | 7.221 | 7.32 | 7.07 | 7.22 | 7.07 | 7.024 | −0.128 |
| 202161_at | NM_002741 | PKN1 | 5585 | 0.6679 | 3.134 | 2.98 | 3.154 | 3.26 | 3.5 | 3.474 | 0.152 |
| 202181_at | NM_014734 | KIAA0247 | 9766 | 0.0145 | 7.675 | 7.57 | 7.616 | 7.59 | 7.39 | 7.344 | −0.017 |
| 202258_s_at | U50532 | N4BP2L2 | 10443 | 0.1501 | 8.82 | 8.83 | 8.806 | 8.9 | 8.91 | 8.873 | 0.032 |
| 202259_s_at | NM_014887 | N4BP2L2 | 10443 | −0.1926 | 6.199 | 6.27 | 6.319 | 6.17 | 6.43 | 6.271 | 0.013 |
| 202273_at | NM_002609 | PDGFRB | 5159 | −0.1669 | 3.527 | 3.47 | 3.457 | 3.18 | 3.3 | 3.03 | −0.182 |
| 202301_s_at | BE396879 | RSRC2 | 65117 | 0.0912 | 8.034 | 7.99 | 8.035 | 7.94 | 8.47 | 8.446 | −0.021 |
| 202302_s_at | NM_023032 | RSRC2 | 65117 | 0.1828 | 8.614 | 8.5 | 8.629 | 8.49 | 8.97 | 8.988 | −9E−04 |
| 202333_s_at | AA877765 | UBE2B | 7320 | −0.0886 | 9.652 | 9.65 | 9.544 | 9.54 | 9.56 | 9.436 | −0.112 |
| 202334_s_at | AI768723 | UBE2B | 7320 | −0.0677 | 7.521 | 7.65 | 7.534 | 7.55 | 7.67 | 7.78 | −0.04 |
| 202335_s_at | NM_003337 | UBE2B | 7320 | 0.0841 | 2.863 | 2.7 | 2.686 | 2.59 | 2.62 | 2.533 | −0.144 |
| 202350_s_at | NM_002380 | MATN2 | 4147 | 0.3125 | 4.476 | 4.67 | 4.721 | 4.54 | 5.2 | 5.236 | 0.059 |
| 202354_s_at | AW190445 | GTF2F1 | 2962 | 0.3965 | 6.783 | 6.89 | 6.946 | 7.03 | 6.66 | 6.757 | 0.153 |
| 202355_s_at | BC000120 | GTF2F1 | 2962 | 0.0988 | 7.063 | 7.19 | 7.127 | 7.12 | 6.72 | 6.656 | −0.002 |
| 202356_s_at | NM_002096 | GTF2F1 | 2962 | 0.0537 | 6.29 | 6.33 | 6.25 | 6.18 | 5.7 | 5.891 | −0.096 |
| 202363_at | AF231124 | SPOCK1 | 6695 | 0.3389 | 5.408 | 5.42 | 5.438 | 5.19 | 5.88 | 5.968 | −0.1 |
| 202367_at | NM_001913 | CUX1 | 1523 | −0.0816 | 6.273 | 6.33 | 6.257 | 6.16 | 6.07 | 6.151 | −0.092 |
| 202393_s_at | NM_005655 | KLF10 | 7071 | 0.0303 | 7.556 | 7.63 | 7.453 | 7.87 | 7.96 | 7.757 | 0.069 |
| 202397_at | NM_005796 | NUTF2 | 10204 | 0.2553 | 7.467 | 7.54 | 7.475 | 7.52 | 8.04 | 7.894 | −0.005 |
| 202402_s_at | NM_001751 | CARS | 833 | −0.0476 | 6.956 | 6.97 | 7.138 | 7.09 | 7.47 | 7.557 | 0.151 |
| 202405_at | BF432332 | TIAL1 | 7073 | 0.0516 | 5.349 | 5.25 | 5.283 | 5.39 | 5.57 | 5.47 | 0.037 |
| 202406_s_at | NM_003252 | TIAL1 | 7073 | 0.0296 | 9.25 | 9.29 | 9.205 | 9.24 | 9.18 | 9.066 | −0.046 |
| 202415_s_at | NM_012267 | HSPBP1 | 23640 | 0.1611 | 5.483 | 5.64 | 5.768 | 5.76 | 5.89 | 6.028 | 0.2 |
| 202424_at | NM_030662 | MAPZK2 | 5605 | 0.0171 | 6.878 | 6.84 | 6.875 | 6.85 | 7.11 | 6.991 | 0.006 |
| 202426_s_at | BE675800 | RXRA | 6256 | 0.1919 | 3.672 | 3.96 | 3.632 | 3.77 | 3.5 | 3.781 | −0.117 |
| 202438_x_at | BF346014 | IDS | 3423 | 0.1304 | 2.956 | 3.34 | 3.254 | 3.17 | 3.47 | 3.427 | 0.066 |
| 202439_s_at | NM_000202 | IDS | 3423 | 0.2224 | 5.648 | 5.29 | 5.521 | 5.31 | 5.49 | 5.523 | −0.05 |
| 202449_s_at | NM_002957 | RXRA | 6256 | 0.1839 | 7.545 | 7.53 | 7.57 | 7.61 | 7.44 | 7.335 | 0.054 |
| 202555_s_at | NM_005965 | MYLK | 4638 | −0.0506 | 5.996 | 5.81 | 5.9 | 5.47 | 6.44 | 6.45 | −0.222 |
| 202575_at | NM_001878 | CRABP2 | 1382 | −0.0471 | 5.958 | 5.82 | 5.958 | 5.97 | 5.61 | 6.062 | 0.076 |
| 202579_x_at | NM_006353 | HMGN4 | 10473 | −0.0038 | 9.327 | 9.24 | 9.379 | 9.32 | 9.44 | 9.376 | 0.064 |
| 202586_at | AA772747 | POLR2L | 5441 | 0.1267 | 3.623 | 3.75 | 3.677 | 3.45 | 3.89 | 3.406 | −0.121 |
| 202598_at | NM_005979 | S100A13 | 6284 | 0.007 | 7.582 | 7.63 | 7.491 | 7.59 | 7.63 | 7.75 | −0.065 |
| 202605_at | NM_000181 | GUSB | 2990 | 0.1212 | 9.372 | 9.33 | 9.262 | 9.22 | 9.09 | 9.097 | −0.11 |
| 202615_at | BF222895 | GNAQ | 2776 | 0.0694 | 8.466 | 8.3 | 8.408 | 8.46 | 8.09 | 8.194 | 0.49 |
| 202639_s_at | AI689052 | RANBP3 | 8498 | 0.1158 | 4.939 | 4.96 | 5.129 | 5.06 | 5.32 | 5.205 | 0.143 |
| 202640_s_at | NM_003624 | RANBP3 | 8498 | 0.0619 | 5.766 | 5.72 | 5.73 | 5.74 | 5.77 | 5.836 | −0.12 |
| 202671_s_at | NM_003681 | PDXK | 8566 | 0136 | 7.068 | 7.18 | 7.216 | 7.19 | 7.82 | 7.944 | 0.079 |
| 202672_s_at | NM_001674 | AAATF3 | 467 | 0.0298 | 7.768 | 7.73 | 7.553 | 7.38 | 8.6 | 8.618 | −0.279 |
| 202716_at | NM_002827 | PTPN1 | 5770 | 0.2055 | 7.397 | 7.27 | 7.205 | 7.29 | 7.1 | 7.005 | −0.087 |
| 202733_at | NM_004199 | P4HA2 | 8974 | −0.001 | 9.269 | 9.43 | 9.273 | 9.34 | 9.24 | 9.183 | −0.046 |
| 202736_s_at | AA112507 | LSM4 | 25804 | −0.0328 | 9.199 | 9.36 | 9.298 | 9.18 | 9.14 | 9.325 | −0.038 |
| 202737_s_at | NM_012321 | LSM4 | 25804 | 0.0279 | 8.744 | 8.61 | 8.664 | 8.75 | 8.48 | 8.593 | 0.027 |
| 202740_at | NM_000666 | ACY1 | 95 | 0.2267 | 6.839 | 6.92 | 6.786 | 6.77 | 6.88 | 7.153 | −0.101 |
| 207255_s_at | AI354854 | GPC1 | 2817 | −0.2258 | 3.903 | 3.94 | 3.977 | 3.9 | 4.15 | 4.097 | 0.017 |
| 202756_s_at | NM_002081 | GPC1 | 2817 | −0.144 | 7.075 | 6.97 | 6.913 | 6.82 | 6.97 | 6.863 | −0.155 |
| 202759_s_at | BE879367 | AKAP2 /// PALM2 /// | 11217 /// 114299 /// | 0.1219 | 6.446 | 6.11 | 6.222 | 6.06 | 6.72 | 7.079 | −0.134 |
| | | PALM2-AKAP2 | 445815 | | | | | | | | |
| 202760_s_at | NM_007203 | PALM2-AKAP2 | 445815 | 0.4104 | 7.314 | 6.97 | 7.082 | 7.06 | 8.13 | 8.053 | −0.073 |
| 202761_s_at | NM_015180 | SYNE2 | 23224 | 0.0312 | 7.814 | 7.69 | 7.715 | 7.69 | 7.1 | 7.167 | −0.048 |
| 202797_at | NM_014016 | SACM1L | 22908 | −0.2906 | 8.463 | 8.49 | 8.35 | 8.6 | 7.83 | 7.849 | −0.002 |
| 202806_at | NM_004395 | DBN1 | 1627 | 0.1296 | 6.169 | 6.07 | 5.924 | 5.78 | 6.52 | 6.569 | −0.266 |
| 202833_s_at | NM_000295 | SEPINA1 | 5265 | −0.0919 | 5.669 | 5.8 | 4.968 | 5.87 | 5.28 | 4.453 | −0.316 |
| 202865_at | AI695173 | DNAJB12 | 54788 | 0.0569 | 3.726 | 3.8 | 3.569 | 3.66 | 3.94 | 3.515 | −0.148 |
| 202866_at | BG283782 | DNAJB12 | 54788 | −0.0169 | 6.741 | 6.79 | 6.918 | 6.94 | 6.81 | 6.798 | 0.165 |
| 202867_s_at | NM_017626 | DNAJB12 | 54788 | −0.576 | 6.356 | 6.28 | 6.025 | 6.21 | 5.98 | 6.089 | −0.199 |
| 202905_x_at | AI796269 | NBN | 4683 | −0.45 | 8.105 | 8.06 | 8.109 | 8.32 | 8.16 | 8.322 | 0.131 |
| 202906_s_at | AP049895 | NBN | 4683 | 0.1477 | 6.419 | 6.47 | 6.554 | 6.65 | 6.99 | 7.071 | 0.156 |
| 202907_s_at | NM_002485 | NBN | 4683 | −0.0256 | 7.123 | 7.07 | 7.077 | 6.98 | 6.99 | 7.129 | −0.07 |
| 202918_s_at | AF151853 | MOBKL3 | 25843 | −0.255 | 9.063 | 9.08 | 9.25 | 9.1 | 9.01 | 9.077 | 0.101 |
| 202919_at | NM_015387 | MOBKL3 | 25843 | 0.0403 | 8.069 | 8.05 | 8.045 | 8.01 | 7.96 | 7.905 | −0.28 |
| 202934_at | AI761561 | HK2 | 3099 | 0.0849 | 6.277 | 5.93 | 6.202 | 6.31 | 6.98 | 6.996 | 0.149 |
| 202950_at | NM_001889 | CRYZ | 1429 | −0.1832 | 7.175 | 7.48 | 7.45 | 7.41 | 6.21 | 5.75 | 0.089 |
| 202996_at | NM_021173 | POLD4 | 57804 | 0.0795 | 5.826 | 5.97 | 6.035 | 6.04 | 5.76 | 5.546 | 0.139 |
| 203020_at | NM_014857 | RABGAP1L | 9910 | 0.1314 | 7.23 | 7.25 | 7.05 | 7.22 | 7.11 | 6.968 | −0.101 |
| 203038_at | NM_002844 | PTPRK | 5796 | 0.2856 | 9.853 | 9.79 | 9.88 | 9.89 | 10.5 | 10.6 | 0.053 |
| 203051_at | NM_014952 | BAHD1 | 22893 | 0.0071 | 3.994 | 3.99 | 3.925 | 3.98 | 3.63 | 4.042 | −0.041 |
| 203064_s_at | NM_004514 | FOXK2 | 3607 | 0.2673 | 5.351 | 5.34 | 5.711 | 5.42 | 5.94 | 5.806 | 0.221 |
| 203081_at | NM_020248 | CTNNBIP1 | 56998 | 0.094 | 6.426 | 6.25 | 6.397 | 6.27 | 5.86 | 5.861 | −0.002 |
| 203082_at | NM_014753 | BMS1 | 9790 | 0.0521 | 6.722 | 6.79 | 6.879 | 6.64 | 6.86 | 7.036 | 0.003 |
| 203107_x_at | NM_002952 | RPS2 | 6187 | −0.0266 | 13.02 | 13 | 13.03 | 13.1 | 12.9 | 12.92 | 0.39 |
| 203113_s_at | NM_001960 | EEF1D | 1936 | −0.2511 | 10.97 | 11 | 11 | 11 | 10.8 | 10.87 | 0.007 |
| 203173_s_at | AW080196 | C16orf62 | 57020 | −0.0869 | 5.709 | 5.49 | 5.809 | 5.65 | 5.01 | 5.297 | 0.133 |
| 203179_at | NM_000155 | GALT | 2592 | 0.2205 | 5.935 | 5.94 | 5.818 | 5.76 | 5.22 | 5.333 | −0.15 |
| 203188_at | NM_006876 | B3GNT1 | 11041 | 0.1324 | 6.263 | 6.14 | 5.959 | 6.19 | 6.35 | 6.011 | −0.124 |
| 203193_at | NM_004451 | ESRRA | 2101 | −0.1007 | 4.151 | 4.37 | 4.374 | 4.26 | 4.35 | 4.47 | 0.054 |
| 203231_s_at | AW235612 | ATXN1 | 6310 | −0.0724 | 4.32 | 4.38 | 4.267 | 4.47 | 4.51 | 4.351 | −0.037 |
| 203232_s_at | NM_000332 | ATXN1 | 6310 | −0.2047 | 6.275 | 6.27 | 6.442 | 6.45 | 6.39 | 6.324 | 0.172 |
| 203234_at | NM_003364 | UPP1 | 7378 | −0.0385 | 6.022 | 5.87 | 6.314 | 6.48 | 8.05 | 7.807 | 0.451 |
| 203258_at | NM_006442 | DRAP1 | 10589 | 0.0778 | 6.019 | 5.96 | 6.094 | 6.27 | 6.56 | 6.507 | 0.196 |
| 203297_s_at | BG029530 | JARID2 | 3720 | −0.1401 | 5.686 | 5.75 | 5.629 | 5.75 | 6.25 | 6.261 | −0.026 |
| 203298_s_at | NM_004973 | JARID2 | 3720 | 0.1106 | 6.242 | 6.51 | 6.492 | 6.29 | 7.2 | 7.01 | 0.013 |
| 203321_s_at | AK022588 | ADNP2 | 22850 | 0.0812 | 8.109 | 8.07 | 7.978 | 8.06 | 7.97 | 7.865 | −0.069 |
| 203322_at | AU145934 | ADNP2 | 22850 | 0.2053 | 6.711 | 6.67 | 6.576 | 6.7 | 6.69 | 6.61 | −0.053 |
| 203323_at | BF197655 | CAV2 | 858 | 0.3339 | 10.76 | 10.8 | 10.72 | 10.7 | 11.1 | 10.94 | −0.06 |
| 203324_s_at | NM_001233 | CAV2 | 858 | −0.049 | 10.45 | 10.4 | 10.43 | 10.4 | 10.8 | 10.71 | 0.009 |
| 203334_at | NM_004941 | DHX8 | 1659 | 0.0519 | 6.094 | 6.17 | 5.959 | 6.04 | 5.85 | 5.95 | −0.13 |
| 203366_at | NM_002693 | POLG | 5428 | 0.1616 | 6.761 | 6.81 | 6.875 | 6.93 | 7.55 | 7.225 | 0.115 |
| 203368_at | NM_015513 | CRELD1 | 7898 | −0.1144 | 6.565 | 6.5 | 5.935 | 6.24 | 5.95 | 5.931 | −0.445 |
| 203406_at | NM_005926 | MFAF1 | 4236 | −0.062 | 8.582 | 8.72 | 8.74 | 8.69 | 8.3 | 8.526 | 0.065 |
| 203456_at | NM_007213 | PRAF2 | 11230 | 0.0175 | 6.046 | 6 | 6.105 | 5.99 | 5.97 | 5.902 | 0.024 |
| 203458_at | AI951454 | SPR | 6697 | 0.0243 | 6.062 | 6.16 | 5.919 | 5.83 | 5.36 | 5.625 | 0.236 |
| 203499_at | NM_004431 | EPHA2 | 1969 | −0.0014 | 6.061 | 5.93 | 5.997 | 5.87 | 6.98 | 6.902 | −0.059 |
| 203511_s_at | AF041432 | TRAPPC3 | 27095 | −0.1571 | 8.051 | 8.12 | 8.036 | 8.18 | 8.17 | 8.211 | 0.023 |
| 203512_at | NM_014408 | TRAPPC3 | 27095 | −0.0742 | 7.468 | 7.42 | 7.589 | 7.34 | 7.62 | 7.557 | 0.019 |
| 203515_s_at | NM_006556 | PMVK | 10654 | −0.0596 | 6.624 | 6.67 | 6.767 | 6.64 | 6.49 | 6.631 | 0.056 |
| 203557_s_at | NM_000281 | PCBD1 | 5092 | 0.434 | 6.476 | 6.57 | 6.456 | 6.6 | 6.14 | 6.245 | 0.007 |
| 203561_at | NM_021642 | FCGR2A | 2212 | 0.0733 | 2.716 | 2.81 | 2.715 | 2.71 | 2.69 | 2.676 | −0.05 |
| 203571_s_at | NM_006829 | C10orf116 | 10974 | −0.0812 | 8.29 | 8.28 | 8.337 | 8.48 | 7.83 | 7.086 | 0.127 |
| 203627_at | AI830598 | IGF3R | 3480 | −0.1519 | 6.855 | 6.84 | 6.698 | 6.78 | 6.67 | 6.679 | −0.108 |
| 203628_at | H05812 | IGF1R | 3480 | 0.1342 | 7.91 | 7.9 | 7.732 | 7.77 | 7.3 | 7.354 | −0.152 |
| 203710_at | NM_002222 | ITPR1 | 3708 | −0.092 | 4.246 | 4.7 | 4.312 | 4.58 | 4.59 | 4.549 | −0.027 |
| 203778_at | NM_005908 | MANBA | 4126 | 0.0556 | 5.815 | 6.04 | 5.827 | 6.05 | 6.03 | 6.16 | 0.011 |
| 203792_x_at | BC004558 | PCGF2 | 7703 | 0.0189 | 4.099 | 3.62 | 3.837 | 3.67 | 3.99 | 4.047 | −0.105 |
| 203793_x_at | NM_007144 | PCGF2 | 7703 | −0.2742 | 4.512 | 4.43 | 4.271 | 4.18 | 4.53 | 4.205 | −0.243 |
| 203810_at | BG252490 | DNA3B4 | 11080 | −0.0555 | 6.363 | 6.52 | 6.369 | 6.58 | 6.25 | 5.943 | 0.03 |
| 203811_s_at | NM_007034 | DNAJB4 | 11080 | −0.0308 | 6.773 | 6.57 | 6.564 | 6.43 | 6.18 | 6.478 | −0.175 |
| 203818_s_at | NM_006802 | SF3A3 | 10946 | 0.1622 | 7.309 | 7.19 | 7.341 | 7.22 | 7.42 | 7.275 | 0.029 |
| 203830_at | NM_022344 | C17orf75 | 64149 | −0.0234 | 6.717 | 5.98 | 6.781 | 6.69 | 6.62 | 6.695 | −0.114 |
| 203860_at | NM_000282 | PCCA | 5095 | −0.0742 | 5.332 | 5.6 | 5.606 | 5.66 | 5.26 | 5.201 | 0.168 |
| 203876_s_at | AI761713 | MMP11 | 4320 | 0.0197 | 3.051 | 2.9 | 3.174 | 3 | 2.89 | 2.982 | 0.113 |
| 203877_at | NM_005940 | MMP11 | 4320 | −0.0677 | 2.74 | 2.73 | 3.1 | 2.76 | 2.75 | 2.947 | 0.191 |
| 203878_s_at | NM_005940 | MMP11 | 4320 | −0.042 | 3.6 | 3.38 | 3.43 | 3.33 | 3.27 | 3.32 | −0.109 |
| 203886_s_at | NM_001998 | FBLN2 | 2199 | 0.0311 | 2.988 | 2.93 | 3.049 | 3.41 | 3.09 | 2.725 | 0.269 |
| 203905_at | NM_002582 | PARN | 5073 | −0.025 | 6.854 | 7.08 | 7.039 | 6.81 | 6.75 | 6.565 | −0.043 |
| 203963_at | NM_001218 | CA12 | 771 | −0.1826 | 7.515 | 7.51 | 7.596 | 8.05 | 7.62 | 6.618 | 0.309 |
| 203966_s_at | NM_021003 | PPM1A | 5494 | 0.1447 | 7.846 | 7.76 | 7.687 | 7.78 | 7.73 | 7.898 | −0.072 |
| 203969_at | AU157140 | PEX3 | 8504 | 0.0227 | 3.306 | 3.1 | 3.365 | 3.3 | 3.23 | 3.19 | 0.128 |
| 203970_s_at | NM_003630 | PEX3 | 8504 | −0.0016 | 7.94 | 8.14 | 8.088 | 8.1 | 7.7 | 7.468 | 0.055 |
| 203972_s_at | AB035307 | PEX3 | 8504 | −0.0017 | 7.561 | 7.68 | 7.681 | 7.78 | 7.43 | 7.414 | 0.311 |
| 204023_at | NM_002916 | RFC4 | 5984 | 0.0073 | 9.979 | 9.91 | 9.891 | 9.87 | 9.96 | 10.05 | −0.064 |
| 204030_s_at | NM_014575 | SCHIP1 | 29970 | −0.0597 | 7.812 | 7.88 | 7.57 | 7.45 | 7.79 | 7.846 | −0.337 |
| 204053_x_at | U96180 | PTEN | 5728 | −0.0118 | 8.389 | 8.44 | 8.454 | 8.47 | 8.38 | 8.244 | 0.05 |
| 204054_at | NM_000314 | PTEN | 5728 | −0.0113 | 4.187 | 4.05 | 4.341 | 4.04 | 4.14 | 4.26 | 0.071 |
| 204065_at | NM_004854 | CHST10 | 9486 | 0.1035 | 4.014 | 3.82 | 3.759 | 3.87 | 3.96 | 3.892 | −0.103 |
| 204068_at | NM_006281 | STK3 | 6788 | −0.044 | 8.26 | 8.47 | 8.33 | 8.35 | 8.93 | 8.945 | −0.023 |
| 204095_s_at | AL521391 | ELL | 8178 | 0.5194 | 3.069 | 3.04 | 3.345 | 3.13 | 3.62 | 3.417 | 0.184 |
| 204096_s_at | AL136771 | ELL | 8178 | 0.1988 | 2.891 | 2.99 | 3.112 | 3.01 | 2.9 | 3.027 | 0.121 |
| 204163_at | NM_007046 | EMILIN1 | 11117 | 0.0875 | 2.466 | 2.63 | 2.756 | 2.75 | 2.58 | 2.509 | 0.204 |
| 204170_s_at | NM_001827 | CKS2 | 1164 | −0.215 | 9.213 | 9.34 | 9.174 | 9.17 | 8.53 | 8.64 | −0.104 |
| 204173_at | NM_002475 | MYL6B | 140465 | 0.0019 | 8.644 | 8.68 | 8.602 | 8.5 | 8.25 | 8.178 | −0.11 |
| 204190_at | NM_005800 | USPL1 | 10208 | 0.1201 | 7.115 | 7.02 | 7.053 | 7.04 | 7.04 | 7.163 | −0.025 |
| 204202_at | NM_017604 | IQCE | 23288 | 0.3303 | 3.974 | 3.79 | 3.732 | 3.8 | 3.95 | 3.949 | −0.115 |
| 204238_s_at | NM_006443 | C6orf108 | 10591 | 0.0774 | 7.118 | 7.49 | 7.279 | 7.2 | 6.9 | 7.247 | −0.063 |
| 204292_x_at | NM_000455 | STK11 | 6794 | 0.2463 | 3.355 | 4.02 | 3.53 | 3.61 | 3.7 | 3.595 | −0.118 |
| 204306_s_at | NM_004357 | CD151 | 977 | −0.0746 | 7.006 | 7.12 | 7.159 | 7.06 | 7.21 | 6.952 | 0.044 |
| 204402_at | NM_012265 | RHBDD3 | 25807 | 0.1313 | 3.467 | 3.43 | 3.919 | 3.33 | 3.65 | 3.754 | 0.374 |
| 204441_s_at | NM_002689 | POLA2 | 23649 | 0.0974 | 6.113 | 6.19 | 6.28 | 6.11 | 5.98 | 6.087 | 0.041 |
| 204442_x_at | NM_003573 | LTBP4 | 8425 | 0.1085 | 4.559 | 4.27 | 4.148 | 3.97 | 4.19 | 4.204 | −0.354 |
| 204503_at | NM_001988 | EVPL | 2125 | −0.0382 | 3.48 | 3.35 | 3.39 | 3.38 | 3.4 | 3.455 | −0.031 |
| 204508_s_at | BC001012 | CA12 | 771 | −0.2696 | 4.781 | 4.49 | 4.879 | 5.1 | 4.98 | 4.034 | 0.353 |
| 204509_at | NM_017689 | CA12 | 771 | −0.1345 | 3.299 | 3.12 | 3.393 | 3.29 | 3.41 | 3.348 | 0.131 |
| 204537_s_at | NM_004961 | GABRE | 2564 | 0.0801 | 4.088 | 3.77 | 4.112 | 3.81 | 4.04 | 3.906 | 0.033 |
| 204539_s_at | NM_014246 | CELSR1 | 9620 | 0.0173 | 2.921 | 2.9 | 2.819 | 2.78 | 2.93 | 2.771 | −0.107 |
| 204625_s_at | BF115658 | ITGB3 | 3690 | 0.2381 | 3.055 | 3 | 3.036 | 3.23 | 3.1 | 3.184 | 0.104 |
| 204626_s_at | J02703 | ITGB3 | 3690 | −0.254 | 3.25 | 3.04 | 2.972 | 3.08 | 3.26 | 3.187 | −0.117 |
| 204627_s_at | M35999 | ITGB3 | 3690 | 0.0728 | 2.695 | 2.77 | 2.648 | 2.68 | 2.64 | 2.626 | −0.065 |
| 204628_s_at | NM_000212 | ITGB3 | 3690 | −0.0018 | 2.993 | 2.85 | 3.002 | 3.38 | 2.82 | 3.001 | 0.267 |
| 204691_x_at | NM_003560 | PLA2G6 | 8398 | −0.3938 | 3.615 | 3.67 | 3.325 | 3.57 | 3.45 | 3.409 | −0.196 |
| 204762_s_at | BE670563 | GNAO1 | 2775 | −0.2104 | 2.848 | 2.96 | 2.778 | 2.95 | 2.82 | 2.752 | −0.042 |
| 204763_s_at | NM_020988 | GNAO1 | 2775 | −0.1524 | 3.539 | 3.32 | 3.066 | 3.41 | 3.23 | 3.265 | −0.189 |
| 204773_at | NM_004512 | IL11RA | 3590 | 0.5785 | 5.109 | 5.33 | 5.06 | 4.94 | 4.39 | 4.718 | −0.222 |
| 204785_x_at | NM_000874 | IFNAR2 | 3455 | −0.0944 | 6.874 | 6.69 | 6.756 | 6.63 | 6.43 | 6.564 | −0.089 |
| 204786_s_at | L41944 | IFNAR2 | 3455 | 0.1973 | 4.714 | 4.23 | 4.899 | 4.8 | 4.58 | 4.854 | 0.379 |
| 204802_at | NM_004165 | RRAD | 6236 | 0.3316 | 3.732 | 3.49 | 3.14 | 3.4 | 3.44 | 3.49 | −0.341 |
| 204803_s_at | NM_004165 | RRAD | 6236 | 0.373 | 5.372 | 5.22 | 5.141 | 4.49 | 5 | 5.143 | −0.477 |
| 204857_at | NM_003550 | MAD1L1 | 8379 | 0.0837 | 6.159 | 6.09 | 5.954 | 5.94 | 5.89 | 6.207 | −0.18 |
| 204883_s_at | AI968626 | HUS1 | 3364 | 0.1585 | 6.522 | 6.36 | 6.352 | 6.26 | 6.75 | 6.913 | −0.139 |
| 204884_s_at | NM_004507 | HUS1 | 3364 | 0.062 | 2.806 | 2.77 | 2.845 | 2.97 | 2.77 | 2.77 | 0.121 |
| 204945_at | NM_002846 | PTPRN | 5798 | 0.1056 | 2.727 | 2.74 | 2.691 | 2.79 | 2.57 | 2.772 | 0.006 |
| 204962_s_at | NM_001809 | CENPA | 1058 | 0.2324 | 5.709 | 5.26 | 5.37 | 5.42 | 5.2 | 5.276 | −0.087 |
| 204981_at | NM_002555 | SLC22A18 | 5002 | 0.1755 | 6.32 | 6.31 | 6.597 | 6.57 | 6.41 | 6.008 | 0.266 |
| 204995_at | AL567411 | CDK5R1 | 8851 | 0.1596 | 3.435 | 3.33 | 3.251 | 3.14 | 4.14 | 3.937 | −0.187 |
| 204996_s_at | NM_003885 | CDK5R1 | 8851 | −0.1657 | 2.798 | 2.92 | 2.882 | 2.82 | 2.56 | 2.832 | −0.01 |
| 205003_at | NM_014705 | DOCK4 | 9732 | −0.1147 | 4.305 | 4.29 | 4.211 | 4.35 | 4.75 | 4.513 | −0.016 |
| 205005_s_at | AW293531 | NMT2 | 9397 | 0.1436 | 4.491 | 4.34 | 4.331 | 4.48 | 4.55 | 4.513 | −0.013 |
| 205006_s_at | NM_004808 | NMT2 | 9397 | −0.0081 | 4.439 | 4.37 | 4.176 | 4.1 | 4.52 | 4.223 | −0.266 |
| 205048_s_at | NM_003832 | PSPH | 5723 | −0.4672 | 10.39 | 10.4 | 10.53 | 10.5 | 10.1 | 10.32 | 0.113 |
| 205089_at | NM_003416 | ZNF7 | 7553 | 0.0836 | 7.001 | 7.16 | 7.227 | 6.99 | 7.57 | 7.611 | 0.03 |
| 205092_x_at | NM_014950 | ZBTB1 | 22890 | 0.1465 | 3.536 | 3.6 | 3.85 | 3.77 | 4.06 | 3.582 | 0.24 |
| 205093_at | NM_014935 | PLEKHA6 | 22874 | −0.116 | 3.207 | 3.58 | 3.237 | 3.25 | 3.04 | 3.456 | −0.151 |
| 205133_s_at | NM_002157 | HSPE1 | 3336 | −0.237 | 10.14 | 10.1 | 9.929 | 9.92 | 9.58 | 9.584 | −0.215 |
| 205141_at | NM_001145 | ANG | 283 | −0.1128 | 3.846 | 3.81 | 4.077 | 3.78 | 3.41 | 3.402 | 0.099 |
| 205158_at | NM_002937 | RNASE4 | 6038 | −0.0693 | 3.794 | 4.04 | 3.522 | 3.74 | 3.43 | 3.033 | −0.288 |
| 205163_at | NM_013292 | MYLPF | 29895 | 0.1804 | 3.264 | 3.42 | 3.075 | 3.08 | 3.1 | 3.152 | −0.264 |
| 205175_at | NM_000221 | KHK | 3795 | 0.2535 | 3.106 | 3.02 | 3.379 | 3.21 | 3.03 | 2.989 | 0.229 |
| 205176_s_at | NM_014288 | ITGB3BP | 23421 | 0.1063 | 9.787 | 9.83 | 9.907 | 9.78 | 9.37 | 9.324 | 0.035 |
| 205189_s_at | NM_000136 | FANCC | 2176 | 0.0179 | 4.03 | 4.23 | 4.317 | 4.29 | 4.12 | 3.932 | 0.174 |
| 205194_at | NM_004577 | PSPH | 5723 | −0.2405 | 6.447 | 6.61 | 6.521 | 6.61 | 6.96 | 6.749 | 0.042 |
| 205227_at | NM_002182 | IL1RAP | 3556 | −0.283 | 6.858 | 6.91 | 6.91 | 6.76 | 6.34 | 6.191 | −0.047 |
| 205263_at | AF082283 | BCL10 | 8915 | −0.3213 | 7.976 | 7.78 | 7.551 | 7.74 | 8.08 | 7.908 | −0.23 |
| 205274_at | U87964 | GTPBP1 | 9567 | −0.3599 | 3.101 | 3.2 | 3.028 | 3.15 | 2.81 | 2.99 | −0.061 |
| 205275_at | BE866976 | GTPBP1 | 9567 | −0.0255 | 3.321 | 3.69 | 3.329 | 3.35 | 3.65 | 3.535 | −0.165 |
| 205276_s_at | NM_004286 | GTPBP1 | 9567 | −0.1349 | 3.183 | 3.3 | 3.102 | 3.23 | 3.27 | 3.193 | −0.079 |
| 205292_s_at | NM_002137 | HNRNPA2B1 | 3181 | −0.1804 | 10.72 | 10.8 | 10.72 | 10.6 | 10.5 | 10.62 | −0.078 |
| 205293_x_at | AB017120 | BAIAP2 | 10458 | −0.013 | 3.795 | 3.84 | 3.641 | 3.87 | 4.05 | 4.267 | −0.061 |
| 205294_at | NM_017450 | BAIAP2 | 10458 | 0.2796 | 3.748 | 3.59 | 3.447 | 3.59 | 3.59 | 3.761 | −0.153 |
| 205320_at | NM_005883 | APC2 | 10297 | 0.1638 | 3.058 | 3 | 3.134 | 3.04 | 2.91 | 3.105 | 0.061 |
| 205341_at | NM_014601 | EHD2 | 30846 | 0.2157 | 3.767 | 3.78 | 3.742 | 3.62 | 3.75 | 3.696 | −0.091 |
| 205349_at | NM_002068 | GNA15 | 2769 | 0.0167 | 8.708 | 8.67 | 8.881 | 8.69 | 9.38 | 9.43 | 0.096 |
| 205359_at | NM_004274 | AKAP6 | 9472 | −0.0405 | 2.807 | 2.58 | 2.708 | 2.91 | 2.91 | 2.768 | 0.113 |
| 205411_at | NM_006282 | STK4 | 6789 | −0.0301 | 2.873 | 2.97 | 2.838 | 3.26 | 3.49 | 3.183 | 0.128 |
| 205457_at | NM_024294 | C6orf106 | 64771 | −0.1176 | 5.099 | 5.15 | 5.27 | 5.39 | 4.77 | 4.989 | 0.207 |
| 205463_at | NM_002607 | PDGFA | 5154 | 0.5764 | 8.377 | 8.2 | 8.529 | 8.35 | 8.97 | 8.791 | 0.15 |
| 205485_at | NM_000540 | RYR1 | 6261 | −0.038 | 3.381 | 3.46 | 3.658 | 3.67 | 3.11 | 3.376 | 0.242 |
| 205543_at | NM_014278 | HSPA4L | 22824 | 0.0082 | 8.546 | 8.5 | 8.409 | 8.44 | 8.22 | 8.107 | −0.095 |
| 205579_at | NM_000861 | HRH1 | 3269 | 0.291 | 5.561 | 5.64 | 5.553 | 5.66 | 5.52 | 5.577 | 0.008 |
| 205580_at | D28481 | HRH1 | 3269 | −0.0601 | 4.571 | 4.59 | 4.761 | 4.41 | 4.65 | 4.415 | 0.006 |
| 205617_at | NM_000951 | PRRG2 | 5639 | 0.1841 | 4.274 | 4.22 | 4.563 | 4.27 | 4.2 | 4.051 | 0.167 |
| 205640_at | NM_000694 | ALDH3B1 | 221 | −0.1463 | 4.41 | 4.24 | 4.079 | 4.25 | 3.88 | 3.688 | −0.159 |
| 205643_s_at | NM_004576 | PPP2R2B | 5521 | −0.079 | 3.113 | 3.18 | 3.173 | 3.17 | 3.21 | 3.15 | 0.025 |
| 205648_at | NM_003391 | WNT2 | 7472 | −0.1527 | 3.43 | 3.5 | 3.636 | 3.71 | 3.49 | 3.58 | 0.209 |
| 205674_x_at | NM_001680 | FXYD2 | 486 | −0.0275 | 3.406 | 3.41 | 3.73 | 3.26 | 3.29 | 3.232 | 0.088 |
| 205687_at | NM_019116 | UBFD1 | 56061 | −0.0204 | 5.177 | 4.85 | 5.454 | 5.3 | 4.8 | 4.952 | 0.368 |
| 205724_at | NM_000299 | PKP1 | 5317 | −0.173 | 4.233 | 3.95 | 4.374 | 4.2 | 4.81 | 4.939 | 0.195 |
| 205829_at | NM_000413 | HSD17B1 | 3292 | 0.1814 | 4.518 | 4.89 | 5.106 | 4.85 | 5.77 | 5.884 | 0.224 |
| 205858_at | NM_002507 | NGFR | 4804 | 0.0069 | 2.88 | 2.77 | 3.013 | 2.94 | 3.04 | 2.817 | 0.147 |
| 205872_x_at | NM_022359 | PDE4DIP | 9659 | 0.4204 | 3.65 | 4.2 | 3.687 | 4.34 | 5.01 | 4.527 | 0.09 |
| 205873_at | NM_004278 | PIGL | 9487 | 0.0569 | 4.879 | 4.73 | 4.914 | 4.7 | 5.3 | 5.203 | 0.002 |
| 205945_at | NM_000565 | IL6R | 3570 | 0.4607 | 3.927 | 4.03 | 4.056 | 4.2 | 4.35 | 3.727 | 0.151 |
| 205967_at | NM_003542 | HIST1H4A /// | 121504 /// 554313 | −0.3511 | 9.539 | 9.29 | 9.5 | 9.46 | 9.13 | 9.409 | 0.015 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 206066_s_at | NM_002876 | RAD51C | 5889 | 0.031 | 7.338 | 7.42 | 7.476 | 7.24 | 7.08 | 7.259 | −0.022 |
| 206105_at | NM_002025 | AFF2 | 2334 | 0.0191 | 3.458 | 3.29 | 3.093 | 3.18 | 3.15 | 3.256 | −0.239 |
| 206212_at | NM_001869 | CPA2 | 1358 | 0.2019 | 3.302 | 3.35 | 3.35 | 3.47 | 3.53 | 3.371 | 0.088 |
| 206219_s_at | NM_005428 | VAV1 | 7409 | 0.0851 | 2.91 | 2.84 | 2.663 | 3.02 | 2.92 | 3.166 | −0.031 |
| 206236_at | NM_005282 | GPR4 | 2828 | 0.1414 | 2.928 | 2.83 | 2.97 | 2.86 | 2.92 | 2.942 | 0.034 |
| 206248_at | NM_005400 | PRKCE | 5581 | −0.1116 | 3.419 | 3.38 | 3.3 | 3.32 | 3.05 | 3.216 | −0.089 |
| 206275_s_at | NM_014632 | MICAL2 | 9645 | 0.2764 | 3.564 | 3.26 | 3.415 | 3.26 | 3.78 | 3.543 | −0.074 |
| 206316_s_at | NM_014708 | KNTC1 | 9735 | 0.0435 | 7.714 | 7.82 | 7.753 | 7.74 | 7.51 | 7.566 | −0.024 |
| 206322_at | NM_003490 | SYN3 | 8224 | −0.1089 | 3.317 | 3.37 | 3.35 | 3.19 | 3.15 | 3.082 | −0.074 |
| 206324_s_at | NM_014326 | DAPK2 | 23604 | 0.1652 | 3.435 | 3.52 | 3.563 | 3.44 | 3.72 | 3.643 | 0.026 |
| 206342_x_at | NM_006123 | IDS | 3423 | 0.0601 | 6.647 | 6.52 | 6.702 | 6.56 | 6.64 | 6.579 | 0.045 |
| 206357_at | NM_025136 | OPA3 | 80207 | 0.2013 | 3.983 | 3.99 | 3.923 | 3.96 | 4.23 | 3.908 | −0.045 |
| 206400_at | NM_002307 | LGALS7 /// LGALS7B | 3963 /// 653499 | 0.1135 | 8.157 | 8.22 | 8.417 | 8.21 | 8.31 | 8.407 | 0.123 |
| 206410_at | NM_021969 | NR0B2 | 8431 | −0.1125 | 3.033 | 3.14 | 3.091 | 3.46 | 3.19 | 3.209 | 0.19 |
| 206452_x_at | NM_021131 | PPP2R4 | 5524 | 0.0754 | 5.376 | 5.36 | 5.409 | 5.35 | 5.48 | 5.327 | 0.011 |
| 206492_at | NM_002012 | FHIT | 2272 | 0.3118 | 3.007 | 2.82 | 3.275 | 3.02 | 3.22 | 3.209 | 0.231 |
| 206504_at | NM_000782 | CYP24A1 | 1591 | 0.0333 | 3.39 | 3 | 3.175 | 3.02 | 2.96 | 3.029 | −0.099 |
| 206571_s_at | NM_004834 | MAP4K4 | 9448 | 0.0899 | 5.347 | 5.32 | 5.547 | 5.36 | 5.42 | 5.516 | 0.119 |
| 206577_at | NM_003381 | VIP | 7432 | 0.1152 | 2.681 | 2.63 | 2.537 | 2.72 | 2.79 | 2.84 | −0.025 |
| 206582_s_at | NM_005682 | GPRS6 | 9289 | 0.0126 | 4.391 | 4.07 | 4.103 | 4.22 | 4.46 | 4.192 | −0.072 |
| 206709_x_at | NM_005309 | GPT | 2875 | 0.0443 | 3.135 | 3.09 | 3.363 | 2.88 | 3.06 | 3.071 | 0.01 |
| 206720_at | NM_002410 | MGATS | 4249 | 0.2664 | 2.768 | 2.82 | 3.035 | 2.97 | 2.89 | 3.131 | 0.206 |
| 206802_at | NM_016734 | PAX5 | 5079 | −0.0778 | 3.06 | 3.23 | 3.193 | 3.03 | 3.05 | 3.067 | −0.034 |
| 206866_at | NM_001794 | CDH4 | 1002 | 0.0987 | 3.547 | 3.63 | 3.578 | 3.91 | 5.07 | 4.884 | 0.152 |
| 206896_s_at | NM_005145 | GNG7 | 2788 | −0.1546 | 3.951 | 4.01 | 3.83 | 3.81 | 3.97 | 3.93 | −0.158 |
| 206901_at | NM_024323 | C19orf57 | 79173 | −0.0393 | 3.418 | 3.58 | 3.524 | 3.68 | 3.4 | 3.219 | 0.105 |
| 206923_at | NM_002737 | PRKCA | 5578 | −0.129 | 2.961 | 3.21 | 3.105 | 3.06 | 3.14 | 2.893 | −0.002 |
| 206951_at | NM_003545 | HIST1H4A /// | 121504 /// 554313 | 0.0102 | 3.443 | 3.64 | 3.199 | 3.35 | 3.47 | 3.316 | −0.264 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 206976_s_at | NM_006644 | HSPH1 | 10808 | −0.1069 | 8.765 | 8.65 | 8.744 | 8.78 | 8.7 | 8.769 | 0.056 |
| 207040_s_at | NM_003932 | ST13 | 6767 | −0.2203 | 10.22 | 10.3 | 10.12 | 10.2 | 9.65 | 9.624 | −0.086 |
| 207046_at | NM_003548 | HIST1H4A /// | 121504 /// 554313 | 0.025 | 2.995 | 3.17 | 3.258 | 3.11 | 3.75 | 3.833 | 0.105 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 207127_s_at | NM_021644 | HNRNPH3 | 3189 | −0.2432 | 6.838 | 7.04 | 6.94 | 6.9 | 7.28 | 7.266 | −0.017 |
| 207188_at | NM_001258 | CDK3 | 1018 | −0.0081 | 6.323 | 6.31 | 6.348 | 6.31 | 6.18 | 6.161 | 0.011 |
| 207225_at | NM_001088 | AANAT | 15 | −0.233 | 2.626 | 2.79 | 2.545 | 2.5 | 2.44 | 2.539 | −0.19 |
| 207243_x_at | NM_001743 | CALM1 /// CALM2 /// | 801 /// 805 /// 808 | −0.0417 | 11.53 | 11.6 | 11.43 | 11.5 | 11.5 | 11.44 | −0.107 |
| | | CALM3 | | | | | | | | | |
| 207263_x_at | NM_017599 | VEZT | 55591 | 0.0929 | 3.559 | 3.55 | 3.706 | 3.65 | 3.89 | 3.759 | 0.125 |
| 207323_s_at | NM_002385 | MBP | 4155 | 0.1786 | 2.965 | 2.94 | 2.751 | 2.91 | 2.82 | 3.134 | −0.125 |
| 207342_at | NM_001297 | CNGB1 | 1258 | −0.1322 | 2.796 | 2.76 | 2.79 | 2.79 | 2.4 | 2.788 | 0.011 |
| 207358_x_at | NM_012090 | MACF1 | 23499 | −0.1052 | 6.924 | 6.97 | 6.993 | 7.03 | 6.66 | 6.978 | 0.066 |
| 207360_s_at | NM_002531 | NTSR1 | 4923 | −0.2406 | 4.112 | 3.79 | 3.481 | 3.76 | 3.93 | 4.043 | −0.331 |
| 207382_at | NM_003722 | TP63 | 8626 | −0.0247 | 4.285 | 4.02 | 4.309 | 4.31 | 4.18 | 4.364 | 0.158 |
| 207425_s_at | NM_006640 | 10-Sep | 10801 | 0.1178 | 3.424 | 3.36 | 3.576 | 3.39 | 3.25 | 3.249 | 0.094 |
| 207434_s_at | NM_021603 | FXYD2 | 486 | 0.036 | 3.234 | 3.26 | 3.389 | 3.32 | 3.31 | 3.427 | 0.108 |
| 207442_at | NM_000759 | CSF3 | 1440 | −0.0237 | 3.632 | 3.31 | 3.262 | 3.65 | 3.36 | 3.298 | −0.016 |
| 207453_s_at | NM_012266 | DNAJB5 | 25822 | 0.0466 | 3.345 | 3.48 | 3.573 | 3.37 | 3.29 | 3.485 | 0.062 |
| 207518_at | NM_003647 | DGKE | 8526 | −0.2495 | 2.946 | 2.8 | 2.935 | 2.93 | 2.85 | 2.985 | 0.063 |
| 207525_s_at | NM_005716 | GIPC1 | 10755 | −0.0708 | 6.149 | 6.16 | 6.29 | 6.24 | 6.53 | 6.481 | 0.11 |
| 207535_s_at | NM_002502 | NFKB2 | 4791 | −0.0468 | 5.611 | 5.27 | 5.401 | 5.32 | 5.2 | 5.581 | −0.078 |
| 207650_x_at | NM_000955 | PTGER1 | 5731 | −0.2977 | 3.691 | 3.91 | 3.703 | 3.64 | 3.64 | 3.568 | −0.129 |
| 207661_s_at | NM_014631 | SH3PXD2A | 9644 | 0.0581 | 3.607 | 3.39 | 3.439 | 3.45 | 3.4 | 3.581 | −0.054 |
| 207708_at | NM_021628 | ALOXE3 | 59344 | −0.0339 | 3.547 | 3.28 | 3.405 | 3.34 | 3.74 | 3.573 | −0.039 |
| 207711_at | NM_015377 | C20orf117 | 140710 | 0.0234 | 5.741 | 5.78 | 6 | 5.58 | 4.89 | 5.198 | 0.032 |
| 207712_at | NM_001187 | BAGE | 574 | 0.1515 | 2.842 | 2.76 | 2.848 | 2.78 | 2.92 | 2.865 | 0.01 |
| 207714_s_at | NM_004353 | SERPINH1 | 871 | −0.395 | 4.619 | 4.37 | 4.109 | 4.72 | 3.92 | 3.85 | −0.077 |
| 207760_s_at | NM_006312 | NCOR2 | 9612 | 0.2668 | 7.739 | 7.73 | 7.873 | 7.8 | 8.21 | 8.129 | 0.105 |
| 207821_s_at | NM_005607 | PTK2 | 5747 | −0.0897 | 5.308 | 5.33 | 5.214 | 5.34 | 5.03 | 5.091 | −0.041 |
| 207832_at | NM_017451 | BAIAP2 | 10458 | 0.2379 | 3.217 | 3.44 | 3.307 | 3.17 | 3.4 | 3.341 | −0.092 |
| 207838_x_at | NM_020524 | PBXIP1 | 57326 | −0.0921 | 3.23 | 3.19 | 3.313 | 3.4 | 3.22 | 3.198 | 0.148 |
| 207921_x_at | NM_013952 | PAX8 | 7849 | −0.1026 | 2.906 | 2.59 | 2.559 | 2.52 | 2.61 | 2.758 | −0.209 |
| 207923_x_at | NM_013953 | PAX8 | 7849 | 0.2546 | 2.996 | 2.84 | 2.857 | 2.75 | 2.87 | 2.884 | −0.116 |
| 207924_x_at | NM_013992 | PAX8 | 7849 | 0.0923 | 2.52 | 2.48 | 2.517 | 2.53 | 2.75 | 2.631 | 0.025 |
| 207929_at | NM_005314 | GRPR | 2925 | 0.0282 | 3.151 | 3.2 | 3.202 | 3.28 | 3.15 | 3.286 | 0.069 |
| 208002_s_at | NM_007274 | ACOT7 | 11332 | 0.0819 | 7.557 | 7.64 | 7.076 | 7.5 | 7.88 | 8.008 | −0.308 |
| 208003_s_at | NM_006599 | NFAT5 | 10725 | −0.088 | 7.316 | 7.15 | 7.171 | 7.4 | 7.03 | 7.179 | 0.05 |
| 208009_s_at | NM_014448 | ARHGEF16 | 27237 | −0.4979 | 3.981 | 3.75 | 3.885 | 3.71 | 3.68 | 3.88 | −0.066 |
| 208018_s_at | NM_002110 | HCK | 3055 | −0.1754 | 2.769 | 2.76 | 2.665 | 2.78 | 2.8 | 2.891 | −0.045 |
| 208026_at | NM_003540 | HIST1H4A /// | 121504 /// 554313 | 0.0509 | 2.754 | 2.76 | 2.759 | 2.86 | 2.5 | 2.86 | 0.048 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 208031_s_at | NM_000635 | RFX2 | 5990 | −0.0891 | 3.444 | 3.27 | 3.5 | 3.05 | 3.03 | 3.2 | −0.087 |
| 208046_at | NM_003538 | HIST1H4A /// | 121504 /// 554313 | −0.193 | 2.881 | 3.11 | 3.086 | 2.87 | 3.01 | 3.048 | −0.015 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 208076_at | NM_003539 | HIST1H4A /// | 121504 /// 554313 | −0.099 | 3.021 | 2.95 | 2.907 | 2.98 | 2.9 | 2.959 | −0.044 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 208102_s_at | NM_002779 | PSD | 5662 | 0.0832 | 2.881 | 2.85 | 2.574 | 3.25 | 2.99 | 2.931 | 0.047 |
| 208178_x_at | NM_007118 | TRIO | 7204 | 0.31 | 5.759 | 5.75 | 5.52 | 5.75 | 6.46 | 6.316 | −0.119 |
| 208180_s_at | NM_003543 | HIST1H4A /// | 121504 /// 554313 | 0.1134 | 2.838 | 2.8 | 2.76 | 2.74 | 2.83 | 2.896 | −0.068 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 208181_at | NM_003543 | HIST1H4A /// | 121504 /// 554313 | 0.0096 | 2.364 | 2.48 | 2.559 | 2.6 | 2.81 | 2.529 | 0.157 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 208252_s_at | NM_004273 | CHST3 | 9469 | −0.2025 | 3.097 | 3.13 | 3.06 | 2.83 | 3.07 | 3.071 | −0.168 |
| 208272_at | NM_007321 | RANBP3 | 8498 | −0.0206 | 3.191 | 3.22 | 3.481 | 3.41 | 3.09 | 3.361 | 0.236 |
| 208315_x_at | NM_003300 | TRAF3 | 7187 | −0.0869 | 3.7 | 3.79 | 3.741 | 3.77 | 4.02 | 4.199 | 0.007 |
| 208333_at | NM_022363 | LHX5 | 64211 | −0.1813 | 2.72 | 2.78 | 2.967 | 2.94 | 2.95 | 2.823 | 0.206 |
| 208336_s_at | NM_004868 | GPSN2 | 9524 | 0.0584 | 8.572 | 8.75 | 8.708 | 8.54 | 8.3 | 8.261 | −0.039 |
| 208424_s_at | NM_020313 | CIAPIN1 | 57019 | −0.0453 | 6.607 | 6.56 | 6.562 | 6.59 | 6.8 | 6.906 | −0.007 |
| 208441_at | NM_015883 | IGF1R | 3480 | −0.0493 | 3.034 | 2.97 | 3.061 | 3.07 | 2.91 | 2.848 | 0.066 |
| 208580_x_at | NM_021968 | HIST1H4A /// | 121504 /// 554313 | 0.3342 | 5.187 | 5.58 | 5.146 | 5.24 | 5.67 | 5.65 | −0.191 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 208589_at | NM_020389 | TRPC7 | 57113 | 0.1077 | 2.52 | 2.59 | 2.587 | 2.49 | 2.47 | 2.416 | −0.014 |
| 208611_s_at | U83867 | SPTAN1 | 6709 | 0.1139 | 5.287 | 5.5 | 5.538 | 5.11 | 5.18 | 5.512 | −0.069 |
| 208615_s_at | BF795101 | PTP4A2 | 8073 | 0.025 | 8.464 | 8.27 | 8.16 | 8.24 | 8.29 | 8.362 | −0.168 |
| 208616_s_at | U48297 | PTP4A2 | 8073 | 0.1429 | 9.558 | 9.55 | 9.667 | 9.62 | 10 | 10.03 | 0.093 |
| 208617_s_at | AF208850 | PTP4A2 | 8073 | 0.1021 | 8.821 | 8.91 | 8.899 | 8.74 | 8.95 | 8.9 | −0.048 |
| 208633_s_at | W61052 | MACF1 | 23499 | −0.1958 | 5.369 | 5.17 | 5.411 | 5.46 | 5.06 | 5.369 | 0.165 |
| 208634_s_at | AB029290 | MACF1 | 23499 | 0.0363 | 8.065 | 8.03 | 8.138 | 8 | 8.08 | 8.03 | 0.025 |
| 208657_s_at | AF142408 | 10-Sep | 10801 | 0.0822 | 5.453 | 5.59 | 5.57 | 5.57 | 5.43 | 5.362 | 0.049 |
| 208666_s_at | BE866412 | ST13 | 6767 | 0.0328 | 6.979 | 6.86 | 6.915 | 6.95 | 6.01 | 6.004 | 0.013 |
| 208667_s_at | U17714 | ST13 | 6767 | 0.1498 | 9.386 | 9.38 | 9.278 | 9.23 | 8.68 | 8.467 | −0.13 |
| 208684_at | U24105 | COPA | 1314 | −0.3011 | 8.796 | 8.7 | 8.799 | 8.75 | 8.37 | 8.541 | 0.025 |
| 208687_x_at | AF352832 | HSPA8 | 3312 | −0.7298 | 11.2 | 11.3 | 11.24 | 11.2 | 10.7 | 10.76 | 0.002 |
| 208696_at | AF275798 | CCT5 | 22948 | 0.0239 | 10.64 | 10.5 | 10.67 | 10.6 | 10.7 | 10.76 | 0.059 |
| 208713_at | BF724216 | HNRNPUL1 | 11100 | 0.1064 | 7.251 | 7.28 | 7.194 | 7.21 | 7.17 | 7.062 | −0.066 |
| 208730_x_at | AA535244 | RAB2A | 5862 | −0.0088 | 5.582 | 5.57 | 5.562 | 5.55 | 5.54 | 5.311 | −0.019 |
| 208731_at | AW158062 | RAB2A | 5862 | 0.0254 | 8.621 | 8.48 | 8.588 | 8.61 | 8.61 | 8.63 | 0.048 |
| 208732_at | AI743756 | RAB2A | 5862 | 0.2096 | 6.085 | 5.89 | 6.06 | 6.32 | 6.59 | 6.439 | 0.2 |
| 208733_at | AW301641 | RAB2A | 5862 | −0.1433 | 3.249 | 3.43 | 3.518 | 3.67 | 3.75 | 3.709 | 0.255 |
| 208734_x_at | M28213 | RAB2A | 5862 | −0.0343 | 8.263 | 8.37 | 8.496 | 8.55 | 8.61 | 8.644 | 0.209 |
| 208744_x_at | BG403660 | HSPH1 | 10808 | −0.3633 | 8.386 | 8.18 | 8.181 | 8.27 | 7.91 | 8.009 | −0.06 |
| 208756_at | U36764 | EIF3I | 8668 | −0.0968 | 9.75 | 9.73 | 9.705 | 9.75 | 9.71 | 9.794 | −0.013 |
| 208759_at | AF240468 | NCSTN | 23385 | −0.0848 | 6.756 | 6.66 | 6.626 | 6.78 | 6.31 | 6.549 | −0.005 |
| 208760_at | AL031714 | UBE2I | 7329 | 0.215 | 6.477 | 6.24 | 6.44 | 6.54 | 5.99 | 5.651 | 0.129 |
| 208778_s_at | BC000665 | TCP1 | 6950 | 0.0614 | 10.6 | 10.6 | 10.5 | 10.6 | 10.6 | 10.59 | −0.05 |
| 208781_x_at | AF062483 | SNX3 | 8724 | −0.0559 | 9.59 | 9.62 | 9.722 | 9.62 | 9.29 | 9.349 | 0.066 |
| 208791_at | M25915 | CLU | 1191 | 0.0091 | 4.535 | 4.44 | 4.529 | 4.05 | 4.01 | 3.714 | −0.197 |
| 208792_s_at | M25915 | CLU | 1191 | 0.3517 | 4.692 | 4.98 | 5.01 | 4.74 | 4.3 | 3.822 | 0.037 |
| 208806_at | BE379542 | CHD3 | 1107 | 0.1184 | 4.312 | 4.13 | 4.29 | 4.45 | 3.93 | 4.007 | 0.149 |
| 208807_s_at | U91543 | CHD3 | 1107 | −0.0495 | 5.037 | 4.9 | 4.994 | 5.05 | 4.74 | 5.231 | 0.052 |
| 208810_at | AF080569 | DNAJB6 | 10049 | −0.148 | 8.573 | 8.58 | 8.629 | 8.6 | 8.65 | 8.781 | 0.04 |
| 208811_s_at | AF080569 | DNAJB6 | 10049 | −0.0496 | 7.88 | 7.83 | 8.017 | 7.91 | 7.98 | 8.127 | 0.111 |
| 208813_at | BC000498 | GOT1 | 2805 | −0.0133 | 8.391 | 8.33 | 8.559 | 8.44 | 9.12 | 9.193 | 0.141 |
| 208814_at | AA043348 | HSPA4 | 3308 | 0.0142 | 7.783 | 7.76 | 7.611 | 7.72 | 7.22 | 7.356 | −0.108 |
| 208815_x_at | AB023420 | HSPA4 | 3308 | 0.0441 | 9.076 | 9.18 | 9.124 | 9.1 | 9.51 | 9.449 | −0.014 |
| 208820_at | AL037339 | PTK2 | 5747 | 0.0818 | 8.103 | 8 | 8.082 | 8.07 | 8.17 | 8.132 | 0.022 |
| 208837_at | BC000027 | TMED3 | 23423 | −0.0777 | 8.389 | 8.51 | 8.451 | 8.39 | 8.36 | 8.379 | −0.032 |
| 208858_s_at | BC004998 | FAM62A | 23344 | −0.0306 | 7.381 | 7.5 | 7.536 | 7.33 | 7.58 | 7.463 | −0.006 |
| 208874_x_at | BC002545 | PPP2R4 | 5524 | 0.0539 | 5.549 | 5.45 | 5.448 | 5.41 | 5.39 | 5.555 | −0.073 |
| 208888_s_at | AI499095 | NCOR2 | 9612 | 0.0318 | 2.918 | 2.95 | 2.817 | 3.01 | 3.19 | 2.868 | −0.017 |
| 208889_s_at | AI373205 | NCOR2 | 9612 | 0.2683 | 3.462 | 3.38 | 3.32 | 3.52 | 3.46 | 3.697 | 2E−04 |
| 208929_x_at | BC004954 | RPL13 | 6137 | −0.1134 | 12.47 | 12.5 | 12.45 | 12.5 | 12.4 | 12.4 | −0.025 |
| 208968_s_at | BC002568 | CIAPIN1 | 57019 | 0.0678 | 7.977 | 7.95 | 7.969 | 7.75 | 8.26 | 8.328 | −0.104 |
| 208980_s_at | M26880 | RPS27A /// UBB /// | 6233 /// 7314 /// | −0.025 | 12.08 | 12.2 | 12.12 | 12.1 | 12 | 11.97 | −0.002 |
| | | UBC | 7316 | | | | | | | | |
| 208990_s_at | AF132362 | HNRNPH3 | 3189 | −0.4859 | 9.944 | 9.97 | 9.916 | 9.84 | 9.48 | 9.453 | −0.082 |
| 209010_s_at | AI797657 | TRIO | 7204 | 0.1224 | 2.874 | 2.83 | 3.091 | 2.74 | 3.14 | 2.91 | 0.068 |
| 209011_at | BF223718 | TRIO | 7204 | 0.1141 | 5.029 | 4.97 | 4.878 | 4.55 | 5.33 | 5.142 | −0.285 |
| 209012_at | AV718192 | TRIO | 7204 | 0.0171 | 6.968 | 6.77 | 6.883 | 6.59 | 7.26 | 7.305 | −0.136 |
| 209013_x_at | AF091395 | TRIO | 7204 | 0.0271 | 5.885 | 5.35 | 5.674 | 5.8 | 6.24 | 6.242 | 0.119 |
| 209015_s_at | BC002446 | DNAJB6 | 10049 | −0.0469 | 6.132 | 6.2 | 6.319 | 6.17 | 6.23 | 6.335 | 0.079 |
| 209029_at | AF193844 | COPS7A | 50813 | −0.1003 | 6.477 | 6.59 | 6.653 | 6.81 | 6.22 | 6.108 | 0.198 |
| 209036_s_at | BC001917 | MDH2 | 4191 | −0.0253 | 10.43 | 10.5 | 10.59 | 30.5 | 10.6 | 10.7 | 0.069 |
| 209050_s_at | AI421559 | RALGDS | 5900 | 0.1301 | 6.804 | 6.62 | 6.768 | 6.6 | 6.9 | 7.117 | −0.027 |
| 209051_s_at | AF295773 | RALGDS | 5900 | −0.0902 | 4.254 | 4.64 | 4.523 | 4.49 | 4.68 | 4.391 | 0.056 |
| 209072_at | M13577 | MBP | 4155 | −0.017 | 3.043 | 3.22 | 3.307 | 3.2 | 3.18 | 3.254 | 0.124 |
| 209117_at | U79458 | WBP2 | 23558 | 0.0914 | 5.609 | 5.64 | 5.496 | 5.58 | 5.55 | 5.672 | −0.088 |
| 209130_at | BC003686 | SNAP23 | 8773 | −0.1009 | 8.664 | 8.74 | 8.534 | 8.62 | 8.45 | 8.415 | −0.125 |
| 209131_s_at | U55936 | SNAP23 | 8773 | −0.042 | 4.001 | 3.9 | 3.666 | 4.16 | 3.78 | 3.745 | −0.034 |
| 209179_s_at | BC003164 | MBOAT7 | 79143 | 0.1676 | 5.902 | 5.9 | 5.961 | 6 | 5.92 | 6.012 | 0.08 |
| 209214_s_at | BC004817 | EWSR1 | 2130 | −0.0558 | 6.875 | 6.78 | 6.909 | 6.78 | 7.1 | 7.061 | 0.013 |
| 209216_at | BC000464 | WDR45 | 11152 | 0.0823 | 7.864 | 7.82 | 7.947 | 7.76 | 7.73 | 7.773 | 0.01 |
| 209217_s_at | BC000464 | WDR45 | 11152 | −0.0557 | 6.483 | 6.82 | 6.828 | 6.79 | 6.57 | 6.758 | 0.159 |
| 209229_s_at | BC002799 | SAPS1 | 22870 | 0.152 | 3.742 | 3.73 | 3.541 | 3.99 | 3.88 | 4.336 | 0.03 |
| 209263_x_at | BC000389 | TSPAN4 | 7106 | −0.0702 | 7.341 | 7.19 | 7.207 | 7.14 | 7.12 | 7.207 | −0.091 |
| 209264_s_at | AF054841 | TSPAN4 | 7106 | 0.0932 | 6.239 | 6.21 | 6.032 | 6.06 | 5.89 | 5.663 | −0.174 |
| 209282_at | AF309082 | PRKD2 | 25865 | −0.1127 | 4.516 | 4.7 | 4.806 | 4.52 | 4.37 | 4.357 | 0.057 |
| 209380_s_at | AF146074 | ABCCS | 10057 | 0.1592 | 4.966 | 5.32 | 5.177 | 5.01 | 4.93 | 4.912 | −0.048 |
| 209388_at | BC000927 | PAPOLA | 10914 | 0.1517 | 7.384 | 7.55 | 7.25 | 7.48 | 7.76 | 7.669 | −0.104 |
| 209428_s_at | BG420865 | ZFPL1 | 7542 | −0.2592 | 5.074 | 5.45 | 5.794 | 5.41 | 5.64 | 5.204 | 0.341 |
| 209453_at | M81768 | SLC9A1 | 6548 | −0.1032 | 5.214 | 5.29 | 5.293 | 5.11 | 5.22 | 5.171 | −0.049 |
| 209493_at | AF338650 | PDZD2 | 23037 | −0.1087 | 4.928 | 4.63 | 4.942 | 4.83 | 4.6 | 4.993 | 0.106 |
| 209502_s_at | BC002495 | BAIAP2 | 10458 | −0.0399 | 4.447 | 4.88 | 4.837 | 4.78 | 5.07 | 5.174 | 0.146 |
| 209516_at | U50383 | SMYD5 | 10322 | 0.2066 | 3.894 | 3.73 | 3.965 | 3.79 | 3.9 | 3.818 | 0.066 |
| 209552_at | BC001060 | PAX8 | 7849 | −0.0671 | 2.757 | 3.09 | 2.664 | 2.77 | 2.92 | 2.96 | −0.205 |
| 209563_x_at | BC000454 | CALM1 /// CALM2 /// | 801 /// 808 /// 808 | 0.0995 | 9.213 | 9.17 | 9.276 | 9.26 | 9.22 | 9.251 | 0.079 |
| | | CALM3 | | | | | | | | | |
| 209575_at | BC001903 | IL10RB | 3588 | 0.0787 | 6.714 | 6.96 | 6.725 | 6.64 | 6.55 | 6.628 | −0.156 |
| 209579_s_at | AL556619 | MBD4 | 8930 | 0.1433 | 9.152 | 9.08 | 9.199 | 9.27 | 9.33 | 9.309 | 0.118 |
| 209580_s_at | AF114784 | MBD4 | 8930 | 0.1047 | 5.706 | 5.91 | 6.065 | 6.12 | 6.37 | 6.346 | 0.282 |
| 209590_at | AL57414 | BMP7 | 655 | −0.2159 | 4.135 | 4.45 | 4.115 | 4.3 | 3.53 | 3.573 | −0.085 |
| 209591_s_at | M60316 | BMP7 | 655 | −0.0763 | 3.44 | 3.75 | 3.774 | 3.62 | 3.48 | 3.791 | 0.104 |
| 209626_s_at | AL202969 | OSBPL3 | 26031 | 0.1216 | 7.087 | 6.95 | 7.141 | 6.96 | 7.23 | 7.024 | 0.032 |
| 209627_s_at | AY008372 | OSBPL3 | 26031 | 0.5089 | 7.253 | 7.29 | 7.086 | 7.16 | 6.95 | 7.117 | −0.148 |
| 209636_at | BC002844 | NFKB2 | 4791 | −0.1633 | 2.924 | 3.12 | 2.992 | 2.79 | 3.09 | 3.223 | −0.133 |
| 209667_at | BF033242 | CES2 | 8824 | 0.1671 | 8.904 | 8.83 | 8.804 | 8.85 | 8.74 | 8.736 | −0.04 |
| 209668_x_at | D50579 | CES2 | 8824 | 0.0729 | 7.263 | 7.22 | 7.003 | 7.23 | 6.89 | 6.757 | −0.123 |
| 209674_at | D83702 | CRY1 | 1407 | 0.0154 | 6.771 | 6.53 | 6.687 | 6.29 | 6.92 | 6.799 | −0.16 |
| 209675_s_at | BC004242 | HNRNPUL1 | 11100 | 0.1383 | 5.821 | 5.9 | 5.766 | 5.73 | 5.58 | 5.498 | −0.113 |
| 209700_x_at | AB042555 | PDE4DIP | 9659 | 0.3227 | 2.835 | 2.89 | 2.945 | 3.15 | 3.17 | 3.101 | 0.184 |
| 209736_at | AF116571 | SOX13 | 9580 | 0.0949 | 5.125 | 4.89 | 5.004 | 4.99 | 4.89 | 4.946 | −0.013 |
| 209786_at | BC001282 | HMGN4 | 10473 | −0.0869 | 8.141 | 8.03 | 8.065 | 8.11 | 8.19 | 8.289 | 0.003 |
| 209787_s_at | BC001282 | HMGN4 | 10473 | 0.0274 | 9.496 | 9.5 | 9.46 | 9.55 | 9.76 | 9.695 | 0.005 |
| 209805_at | U14658 | PMS2 /// PMS2CL | 441194 /// 5395 | 0.1257 | 5.918 | 5.75 | 6.021 | 5.82 | 6.12 | 6.213 | 0.086 |
| 209807_s_at | U18759 | NFIX | 4784 | 0.2054 | 3.327 | 3.3 | 3.325 | 3.23 | 3.31 | 3.165 | −0.036 |
| 209820_s_at | BC002361 | TBL3 | 10607 | −0.0233 | 4.42 | 4.5 | 4.588 | 4.58 | 4.63 | 4.689 | 0.128 |
| 209834_at | AB017915 | CHST3 | 9469 | −0.3416 | 4.867 | 4.95 | 4.661 | 4.99 | 5 | 4.873 | −0.081 |
| 209849_s_at | AF029669 | RAD51C | 5889 | 0.1405 | 8.933 | 8.89 | 8.809 | 8.84 | 8.97 | 8.957 | −0.088 |
| 209857_s_at | AF245447 | SPHK2 | 56848 | 0.192 | 3.268 | 3.22 | 3.368 | 3.18 | 2.99 | 3.354 | 0.034 |
| 209863_s_at | AF091627 | TP63 | 8626 | 0.1361 | 8.197 | 8.06 | 8.196 | 8.16 | 7.88 | 8.006 | 0.053 |
| 209885_at | BC001338 | RHOD | 29984 | 0.1821 | 8.728 | 8.61 | 8.509 | 8.7 | 9.03 | 9.07 | −0.067 |
| 209899_s_at | AF217197 | PUF60 | 22827 | −0.0245 | 7.195 | 7.25 | 7.243 | 7.25 | 7.39 | 7.451 | 0.024 |
| 209934_s_at | AF225981 | ATP2C1 | 27032 | −0.1963 | 5.89 | 5.84 | 5.951 | 6.2 | 6.51 | 6.419 | 0.21 |
| 209935_at | AF225981 | ATP2C1 | 27032 | −0.0385 | 6.394 | 6.32 | 6.187 | 6.68 | 6.78 | 6.888 | 0.074 |
| 210011_s_at | BC000527 | EWSR1 | 2130 | −0.1925 | 5.685 | 5.69 | 5.663 | 5.55 | 5.77 | 5.878 | −0.078 |
| 210012_s_at | BC000527 | EWSR1 | 2130 | 0.0872 | 3.757 | 3.56 | 3.417 | 3.51 | 3.47 | 3.303 | −0.197 |
| 210043_at | AF334946 | FRMD8 | 83786 | −0.1013 | 3.584 | 3.52 | 3.437 | 3.68 | 3.8 | 3.787 | 0.008 |
| 210083_at | AF071542 | SEMA7A | 8482 | −0.0658 | 3.392 | 3.27 | 3.217 | 3.34 | 3.38 | 3.514 | −0.048 |
| 210110_x_at | AF132363 | HNRNPH3 | 3189 | −0.1029 | 6.401 | 6.38 | 6.128 | 6.21 | 6.47 | 6.536 | −0.22 |
| 210117_at | AF311312 | SPAG1 | 6674 | 0.165 | 6.828 | 6.69 | 6.716 | 6.68 | 7.3 | 7.292 | −0.062 |
| 210120_s_at | BC004349 | RANBP3 | 8498 | −0.0303 | 4.172 | 4.06 | 4.269 | 4.39 | 3.93 | 4.314 | 0.211 |
| 210125_s_at | AF044773 | BANF1 | 8815 | 0.1296 | 8.539 | 8.56 | 8.492 | 8.48 | 8.3 | 8.421 | −0.066 |
| 210130_s_at | AF096304 | TM7SF2 | 7108 | −0.3581 | 4.717 | 4.73 | 4.56 | 4.65 | 4.33 | 4.276 | −0.121 |
| 210136_at | AW070431 | MBP | 4155 | 0.3221 | 7.676 | 7.67 | 7.735 | 7.67 | 7.75 | 7.691 | 0.033 |
| 210150_s_at | BC003355 | LAMA5 | 3911 | 0.0796 | 7.632 | 7.43 | 7.442 | 7.27 | 7.35 | 7.217 | −0.178 |
| 210180_s_at | U87836 | SFRS10 | 6434 | 0.0037 | 7.251 | 7.47 | 7.415 | 7.14 | 7.69 | 7.384 | −0.08 |
| 210211_s_at | AF028832 | HSP90AA1 | 3320 | −0.1476 | 11.04 | 11 | 11 | 10.9 | 10.4 | 10.49 | −0.073 |
| 210233_at | AF167343 | IL1RAP | 3556 | 0.2972 | 5.445 | 5.08 | 5.425 | 5.54 | 5.83 | 5.835 | 0.221 |
| 210255_at | U84138 | RAD51L1 | 5890 | 0.0969 | 3.806 | 3.57 | 3.743 | 3.8 | 3.68 | 3.53 | 0.081 |
| 210305_at | AB042557 | PDE4DIP | 9659 | 0.2974 | 3.154 | 3.07 | 3.264 | 3.11 | 3.57 | 3.303 | 0.075 |
| 210307_s_at | AL136796 | KLHL25 | 64410 | −0.0311 | 5.394 | 5.32 | 5.508 | 5.29 | 5.2 | 5.196 | 0.041 |
| 210331_at | AB048365 | HECW1 | 23072 | −0.023 | 2.781 | 2.87 | 2.965 | 3.03 | 3.04 | 2.99 | 0.177 |
| 210338_s_st | AB034951 | HSPA8 | 3312 | −0.681 | 11.38 | 11.4 | 11.4 | 11.4 | 10.6 | 10.7 | 0.031 |
| 210378_s_at | BC004118 | SSNA1 | 8636 | 0.1232 | 6.268 | 6.22 | 6.248 | 6.29 | 6.16 | 6.323 | 0.023 |
| 210407_at | AF070670 | PPM1A | 5494 | 0.3198 | 7.085 | 6.7 | 6.959 | 6.87 | 7.17 | 7.139 | 0.021 |
| 210426_x_at | U04897 | RORA | 6095 | −0.1355 | 5.04 | 5.09 | 5.076 | 5.13 | 4.9 | 4.701 | 0.039 |
| 210436_at | BC005220 | CCT8 | 10694 | 0.0247 | 3.214 | 2.88 | 3.143 | 2.92 | 3 | 2.871 | −0.013 |
| 210461_s_at | BC002448 | ABLIM1 | 3983 | −0.1569 | 8.146 | 7.94 | 8.267 | 8.29 | 8.5 | 8.57 | 0.233 |
| 210479_s_at | L14611 | RORA | 6095 | −0.2672 | 4.857 | 4.89 | 4.77 | 5.11 | 4.7 | 4.796 | 0.069 |
| 210550_s_at | L26584 | RASGRF1 | 5923 | 0.274 | 3.917 | 3.91 | 3.471 | 3.52 | 3.8 | 3.605 | −0.42 |
| 210554_s_at | BC002486 | CTBP2 | 1488 | −0.2724 | 9.638 | 9.6 | 9.486 | 9.51 | 9.37 | 9.382 | −0.121 |
| 210574_s_at | AF241788 | NUDC | 10726 | −0.0516 | 7.237 | 7.46 | 7.308 | 7.37 | 7.65 | 7.658 | −0.013 |
| 210575_at | AF241788 | NUDC | 10726 | 0.0679 | 2.909 | 3.01 | 2.802 | 2.83 | 2.81 | 3.048 | −0.144 |
| 210588_x_at | L32610 | HNRNPH3 | 3189 | −0.1321 | 7.766 | 7.88 | 7.868 | 7.9 | 8.08 | 8.166 | 0.059 |
| 210628_x_at | AF051344 | LTBP4 | 8425 | 0.201 | 3.619 | 3.61 | 3.804 | 3.74 | 3.4 | 3.437 | 0.155 |
| 210647_x_at | AF102988 | PLA2G6 | 8398 | 0.1885 | 4.089 | 3.92 | 3.731 | 3.72 | 3.59 | 3.913 | −0.282 |
| 210648_x_at | AB047360 | SNX3 | 8724 | 0.0594 | 11.02 | 11.1 | 11.02 | 11.2 | 11.1 | 11.12 | 0.049 |
| 210666_at | AF050145 | IDS | 3423 | 0.1996 | 4.405 | 4.53 | 4.376 | 4.33 | 4.82 | 4.523 | −0.114 |
| 210691_s_at | AF275803 | CACYBP | 27101 | −0.2816 | 8.108 | 8.21 | 8.092 | 8.03 | 7.64 | 7.766 | −0.101 |
| 210735_s_at | BC000278 | CA12 | 771 | −0.3452 | 5.279 | 5.02 | 5.349 | 5.44 | 5.23 | 4.496 | 0.247 |
| 210752_s_at | AF213666 | MLX | 6945 | 0.2086 | 3.885 | 3.79 | 3.451 | 3.89 | 3.95 | 4.268 | −0.164 |
| 210769_at | U18945 | CNGB1 | 1258 | 0.1668 | 3.222 | 3.15 | 3.247 | 3.33 | 3.29 | 3.313 | 0.101 |
| 210780_at | AB006589 | ESR2 | 2100 | −0.161 | 3.257 | 3.31 | 3.128 | 3.24 | 3.02 | 3.274 | −0.097 |
| 210821_x_at | BC002703 | CENPA | 1058 | 0.1054 | 3.787 | 3.74 | 3.503 | 3.86 | 3.42 | 3.676 | −0.083 |
| 210835_s_at | AF222711 | CTBP2 | 1488 | −0.0996 | 9.464 | 9.44 | 9.426 | 9.33 | 9.32 | 9.269 | −0.072 |
| 210878_s_at | BC001202 | JMJD1B | 51780 | −0.0337 | 5.307 | 5.03 | 5.033 | 5.14 | 5.3 | 5.178 | −0.082 |
| 210933_s_at | BC004908 | FSCN1 | 6624 | 0.326 | 6.366 | 6.29 | 6.703 | 6.63 | 6.59 | 6.455 | 0.337 |
| 210956_at | U42387 | PPYR1 | 5540 | −0.0538 | 3.299 | 2.88 | 3.071 | 3 | 3.11 | 3.227 | −0.054 |
| 210957_s_at | L76569 | AFF2 | 2334 | 0.0677 | 2.594 | 2.95 | 2.853 | 2.69 | 2.85 | 2.69 | 0.002 |
| 210984_x_at | U95089 | EGFR | 1956 | 0.2153 | 7.334 | 7.27 | 7.428 | 7.3 | 6.8 | 6.843 | 0.064 |
| 211004_s_at | BC002553 | ALDH3B1 | 221 | −0.215 | 4.797 | 4.88 | 5.062 | 4.93 | 4.78 | 4.351 | 0.155 |
| 211008_s_at | BC000744 | UBE2I | 7329 | −0.1511 | 3 | 3.04 | 2.796 | 3.14 | 2.86 | 2.882 | −0.053 |
| 211015_s_at | L12723 | HSPA4 | 3308 | −0.0521 | 8.78 | 8.89 | 8.813 | 8.94 | 8.97 | 9.01 | 0.044 |
| 211016_x_at | BC002526 | HSPA4 | 3308 | −0.162 | 7.254 | 7.43 | 7.246 | 7.27 | 7.37 | 7.277 | −0.084 |
| 211028_s_at | BC006233 | KHK | 3795 | −0.0029 | 3.482 | 3.39 | 3.366 | 3.36 | 3.16 | 3.304 | −0.074 |
| 211037_s_at | BC006309 | MBOAT7 | 79143 | 0.0082 | 4.175 | 4.13 | 4.19 | 4.11 | 4.14 | 4.132 | −0.002 |
| 211078_s_at | Z25422 | STK3 | 6788 | −0.0235 | 4.335 | 4.11 | 4.357 | 4.2 | 4.67 | 4.408 | 0.053 |
| 211085_s_at | Z25430 | STK4 | 6789 | −0.0876 | 6.8 | 6.73 | 6.701 | 6.79 | 6.7 | 6.769 | −0.021 |
| 211093_at | U31973 | PDE6C | 5146 | −0.0188 | 2.489 | 2.47 | 2.467 | 2.39 | 2.35 | 2.496 | −0.051 |
| 211099_s_at | U58837 | CNGB1 | 1258 | −0.2158 | 2.841 | 2.89 | 2.849 | 2.9 | 3.06 | 2.879 | 0.01 |
| 211117_x_at | AF124790 | ESR2 | 2100 | −0.1363 | 2.803 | 2.73 | 2.768 | 2.91 | 2.58 | 2.85 | 0.073 |
| 211118_x_at | AF051428 | ESR2 | 2100 | −0.1175 | 2.993 | 2.91 | 2.839 | 3 | 2.84 | 2.617 | −0.031 |
| 211119_at | AF060555 | ESR2 | 2100 | −0.013 | 2.68 | 2.61 | 2.553 | 2.65 | 2.67 | 2.522 | −0.048 |
| 211120_x_at | AB006590 | ESR2 | 2100 | −0.2619 | 2.871 | 2.85 | 2.765 | 2.8 | 2.86 | 2.536 | −0.076 |
| 211137_s_at | AF189723 | ATP2C1 | 27032 | 0.0823 | 5.374 | 5.18 | 4.932 | 5.4 | 5.8 | 5.759 | −0.114 |
| 211194_s_at | AB010153 | TP63 | 8626 | 0.2708 | 4.58 | 4.31 | 4.83 | 5.02 | 4.99 | 4.774 | 0.483 |
| 211195_s_at | AF116771 | TP63 | 8626 | −0.1196 | 3.305 | 3.57 | 3.416 | 3.16 | 3.15 | 3.319 | −0.148 |
| 211200_s_at | BC002836 | EFCAB2 | 84288 | 0.0346 | 5.627 | 5.58 | 5.863 | 5.75 | 6.2 | 5.87 | 0.207 |
| 211225_at | U27329 | FUT5 | 2527 | −0.1866 | 3.775 | 3.61 | 3.646 | 3.62 | 3.25 | 3.562 | −0.059 |
| 211259_s_at | BC004248 | BMP7 | 655 | 0.0251 | 3.204 | 3.37 | 3.514 | 3.28 | 3.34 | 3.129 | 0.111 |
| 211260_at | BC004248 | BMP7 | 655 | 0.1678 | 4.063 | 4.13 | 3.935 | 3.92 | 3.72 | 4 | −0.172 |
| 211266_s_at | U35399 | GPR4 | 2828 | −0.0724 | 2.746 | 2.86 | 3.191 | 2.56 | 2.72 | 2.864 | 0.073 |
| 211277_x_at | BC004369 | APP | 351 | −0.2111 | 6.83 | 6.49 | 6.804 | 6.4 | 6.42 | 6.36 | −0.056 |
| 211296_x_at | AB009010 | RPS27A /// UBB /// | 6233 /// 7314 /// | −0.0123 | 12.85 | 12.9 | 12.83 | 12.8 | 12.8 | 12.75 | −0.02 |
| | | UBC | 7316 | | | | | | | | |
| 211323_s_at | L38019 | ITPR1 | 3708 | −0.071 | 3.275 | 3.11 | 3.06 | 3.21 | 3.04 | 3.283 | −0.058 |
| 211345_x_at | AF119850 | EEF1G | 1937 | −0.1387 | 12.4 | 12.4 | 12.4 | 12.4 | 12.3 | 12.28 | −0.029 |
| 211426_x_at | U40038 | GNAQ | 2776 | −0.2915 | 4.288 | 4.18 | 4.077 | 3.84 | 3.9 | 3.708 | −0.275 |
| 211428_at | AF119873 | SERPINA1 | 5265 | −0.0599 | 2.771 | 2.85 | 3.044 | 2.99 | 2.86 | 2.795 | 0.21 |
| 211429_s_at | AF119873 | SERPINA1 | 5265 | −0.1342 | 6.347 | 6.5 | 6.411 | 7.02 | 6.44 | 5.319 | 0.291 |
| 211439_at | AF055270 | SFRS7 | 6432 | −0.2825 | 3.254 | 3.42 | 3.172 | 3.37 | 3.34 | 3.386 | −0.069 |
| 211524_at | U09609 | NFKB2 | 4791 | −0.2675 | 2.879 | 3.13 | 2.95 | 2.91 | 3.02 | 2.94 | −0.074 |
| 211550_at | AF125253 | EGFR | 1956 | −0.1118 | 3.198 | 3.05 | 2.962 | 2.92 | 2.87 | 3.079 | −0.182 |
| 211551_at | K03193 | EGFR | 1956 | −0.1387 | 3.313 | 3.54 | 3.58 | 3.42 | 3.55 | 3.522 | 0.071 |
| 211579_at | U95204 | ITGB3 | 3690 | −0.022 | 2.865 | 2.82 | 2.716 | 2.78 | 2.63 | 2.836 | −0.092 |
| 211607_x_at | U48722 | EGFR | 1956 | −0.0899 | 7.335 | 7.2 | 7.19 | 7.08 | 6.72 | 6.601 | −0.13 |
| 211685_s_at | AF251061 | NCALD | 83988 | 0.1939 | 3.236 | 3.26 | 3.326 | 3.15 | 3.38 | 3.163 | −0.01 |
| 211711_s_at | BC005821 | PTEN | 5728 | 0.0855 | 6.045 | 6.22 | 6.337 | 6.22 | 5.95 | 5.422 | 0.146 |
| 211730_s_at | BC005903 | POLR2L | 5441 | 0.0282 | 9.042 | 9.15 | 8.941 | 8.95 | 9.2 | 9.082 | −0.149 |
| 211751_at | BC005949 | PDE4DIP | 9659 | −0.0481 | 3.547 | 3.44 | 3.458 | 3.68 | 3.83 | 3.299 | 0.079 |
| 211761_s_at | BC005975 | CACYBP | 27101 | 0.0464 | 8.713 | 8.72 | 8.735 | 8.71 | 8.32 | 8.32 | 0.005 |
| 211763_s_at | BC005979 | UBE2B | 7320 | 0.1368 | 7.474 | 7.31 | 7.35 | 7.27 | 7.22 | 7.43 | −0.082 |
| 211782_at | BC006170 | IDS | 3423 | −0.168 | 2.569 | 2.85 | 2.788 | 2.82 | 2.6 | 2.804 | 0.096 |
| 211790_s_at | AF010404 | MLL2 | 8085 | −0.0745 | 2.776 | 2.79 | 2.689 | 2.78 | 2.77 | 2.815 | −0.043 |
| 211828_s_at | AF172268 | TNIK | 23043 | −0.1154 | 3.445 | 3.47 | 3.572 | 3.5 | 2.94 | 3.34 | 0.08 |
| 211834_s_at | AB042841 | TP63 | 8626 | −0.1186 | 3.082 | 3.19 | 3.286 | 3.13 | 3.08 | 3.114 | 0.073 |
| 211907_s_at | AB044555 | PARD6B | 84612 | 0.0234 | 2.832 | 2.98 | 2.74 | 2.81 | 2.86 | 2.776 | −0.13 |
| 211927_x_at | BE963164 | EEF1G | 1937 | −0.0246 | 12.72 | 12.7 | 12.68 | 12.7 | 12.6 | 12.63 | −0.039 |
| 211943_x_at | AL565449 | TPT1 | 7178 | −0.0768 | 13.01 | 13 | 13.03 | 13 | 13 | 12.98 | 0.007 |
| 211968_s_at | AI962933 | HSP90AA1 | 3320 | −0.1304 | 11.15 | 11.1 | 11.1 | 11.2 | 10.6 | 10.66 | 5E−04 |
| 211969_at | BG420237 | HSP90AA1 | 3320 | −0.138 | 11.83 | 11.9 | 11.78 | 11.8 | 11.5 | 11.43 | −0.056 |
| 211984_at | AI653730 | CALM1 /// CALM2 /// | 801 /// 805 /// 808 | 0.2062 | 6.623 | 6.76 | 6.838 | 6.95 | 7.08 | 7.253 | 0.2 |
| | | CALM3 | | | | | | | | | |
| 211985_s_at | AI653730 | CALM1 /// CALM2 /// | 801 /// 805 /// 808 | 0.2721 | 5.191 | 4.96 | 5.183 | 4.93 | 4.9 | 5.162 | −0.018 |
| | | CALM3 | | | | | | | | | |
| 212009_s_at | AL553320 | STIP1 | 10963 | −0.1525 | 8.103 | 8.21 | 8.181 | 8.24 | 7.95 | 8.024 | 0.053 |
| 212012_at | BF342851 | PXDN | 7837 | 0.0117 | 7.678 | 7.79 | 7.656 | 7.47 | 6.88 | 7.01 | −0.169 |
| 212013_at | D86983 | PXDN | 7837 | −0.0179 | 5.938 | 6.01 | 5.917 | 5.92 | 5.5 | 5.321 | −0.058 |
| 212027_at | AI925305 | RBM25 | 58517 | −0.1028 | 8.464 | 8.39 | 8.283 | 8.3 | 8.8 | 8.683 | −0.137 |
| 212028_at | BE466128 | RBM25 | 58517 | −0.0577 | 7.19 | 7.02 | 7.173 | 7.11 | 7.52 | 7.461 | 0.035 |
| 212030_at | BG251218 | RBM25 | 58517 | 0.0415 | 6.912 | 6.9 | 6.814 | 6.79 | 7.24 | 7.244 | −0.104 |
| 212031_at | AV757384 | RBM25 | 58517 | 0.071 | 7.341 | 7.17 | 7.354 | 7.32 | 7.53 | 7.548 | 0.083 |
| 212032_s_at | AL046054 | PTOV1 | 53635 | 0.0021 | 6.522 | 6.52 | 6.48 | 6.26 | 6.08 | 6.061 | −0.153 |
| 212033_at | BF055107 | RBM25 | 58517 | −0.008 | 8.105 | 8.09 | 8.06 | 8.05 | 8.46 | 8.312 | −0.038 |
| 212070_at | AL554008 | GPRS6 | 9289 | 0.2683 | 8.399 | 8.36 | 8.528 | 8.47 | 8.77 | 8.683 | 0.116 |
| 212076_at | AI701430 | MLL | 4297 | −0.1343 | 5.65 | 5.67 | 5.506 | 5.56 | 5.38 | 5.571 | −0.128 |
| 212078_s_at | AA704766 | MLL | 4297 | −0.1345 | 5.729 | 5.82 | 5.737 | 5.72 | 5.68 | 5.721 | −0.046 |
| 212079_s_at | AA715041 | MLL | 4297 | −0.279 | 6.058 | 5.88 | 5.906 | 5.81 | 5.52 | 5.784 | −0.108 |
| 212080_at | AV714029 | MLL | 4297 | 0.2762 | 5.51 | 5.53 | 5.529 | 5.57 | 5.69 | 5.454 | 0.029 |
| 212082_s_at | BE734356 | MYL6 /// MYL6B | 140465 /// 4637 | −0.1346 | 11.93 | 11.9 | 11.76 | 11.7 | 11.7 | 11.83 | −0.175 |
| 212088_at | BF570122 | PMPCA | 23203 | 0.0429 | 7.644 | 7.68 | 7.809 | 7.62 | 7.9 | 7.971 | 0.052 |
| 212125_at | NM_002883 | RANGAP1 | 5905 | −0.1544 | 6.684 | 6.95 | 6.717 | 6.5 | 6.34 | 6.5 | −0.21 |
| 212127_at | BE379408 | RANGAP1 | 5905 | 0.0866 | 5.708 | 5.73 | 5.615 | 5.55 | 5.74 | 5.898 | −0.138 |
| 212191_x_at | AW574664 | RPL13 | 6137 | −0.0329 | 12.71 | 12.7 | 12.68 | 12.7 | 12.7 | 12.64 | −0.032 |
| 212194_s_at | AI418892 | TM9SF4 | 9777 | −0.046 | 6.512 | 6.58 | 6.636 | 6.58 | 6.58 | 6.697 | 0.059 |
| 212198_s_at | AL515964 | TM9SF4 | 9777 | −0.0763 | 5.497 | 5.42 | 5.241 | 5.27 | 5.24 | 5.238 | −0.201 |
| 212221_x_at | AV703259 | IDS | 3423 | 0.1935 | 6.471 | 6.66 | 6.655 | 6.66 | 7.02 | 7.127 | 0.089 |
| 212223_at | AI926544 | IDS | 3423 | 0.2314 | 4.892 | 5.12 | 4.992 | 4.88 | 5.1 | 4.973 | −0.075 |
| 212228_s_at | AC004382 | COQ9 | 57017 | 0.0359 | 7.769 | 7.87 | 7.893 | 7.83 | 7.92 | 8.021 | 0.046 |
| 212255_s_at | AK001684 | ATP2C1 | 27032 | 0.1234 | 6.44 | 6.61 | 6.484 | 6.41 | 6.86 | 6.703 | −0.077 |
| 212259_s_at | BF344265 | PBXIP1 | 57326 | −0.0189 | 4.093 | 4.05 | 4.217 | 4.15 | 3.56 | 3.867 | 0.111 |
| 212284_x_at | BG498776 | TPT1 | 7178 | −0.0348 | 13.21 | 13.2 | 13.13 | 13.2 | 13.2 | 13.12 | −0.046 |
| 212317_at | AK022910 | TNPO3 | 23534 | −0.02 | 7.252 | 7.36 | 7.322 | 7.22 | 7.35 | 7.381 | −0.035 |
| 212318_at | NM_012470 | TNPO3 | 23534 | −0.03 | 7.482 | 7.47 | 7.539 | 7.42 | 7.45 | 7.471 | 0.004 |
| 212338_at | AA621962 | MYO1D | 4642 | 0.3236 | 4.09 | 4.12 | 4.379 | 4.21 | 4.74 | 4.243 | 0.189 |
| 212348_s_at | AB011173 | AOF2 | 23028 | 0.0467 | 6.788 | 6.85 | 6.707 | 6.56 | 6.67 | 5.7 | −0.185 |
| 212367_at | AI799061 | FEM1B | 10116 | 0.228 | 7.862 | 7.95 | 7.803 | 7.86 | 8.2 | 8.252 | −0.075 |
| 212373_at | AW139179 | FEM1B | 10116 | 0.0712 | 5.939 | 5.83 | 5.82 | 5.77 | 6.48 | 6.444 | −0.092 |
| 212374_at | NM_015322 | FEM1B | 10116 | 0.1698 | 4.603 | 4.46 | 4.575 | 4.71 | 5.23 | 5.055 | 0.111 |
| 212394_at | D42044 | KIAA0090 | 23065 | 0.2802 | 5.023 | 4.87 | 4.964 | 4.61 | 4.94 | 4.533 | −0.161 |
| 212395_s_at | BF197122 | KIAA0090 | 23065 | 0.0999 | 5.496 | 5.61 | 5.681 | 5.89 | 5.64 | 5.725 | 0.23 |
| 212396_s_at | AI143233 | KIAA0090 | 23065 | −0.0407 | 5.501 | 5.38 | 5.523 | 5.66 | 5.53 | 5.715 | 0.153 |
| 212411_at | BE747342 | IMP4 | 92856 | 0.1213 | 8.041 | 8.11 | 7.997 | 8.17 | 8.12 | 8.262 | 0.007 |
| 212421_at | AB023147 | C22orf9 | 23313 | 0.0328 | 5.834 | 6.06 | 5.885 | 5.94 | 5.72 | 5.589 | −0.035 |
| 212422_at | AL547263 | PDCD11 | 22984 | 0.0313 | 5.926 | 5.83 | 5.929 | 5.85 | 6.65 | 6.559 | 0.01 |
| 212424_at | AW026194 | PDCD11 | 22984 | 0.2208 | 5.357 | 5.26 | 5.516 | 5.3 | 6.03 | 6.239 | 0.096 |
| 212433_x_at | AA630314 | RPS2 | 6187 | −0.0403 | 12.85 | 12.9 | 12.84 | 12.8 | 12.7 | 12.73 | −0.016 |
| 212445_s_at | AI357376 | NEDD4L | 23327 | 0.0924 | 6.391 | 6.9 | 6.376 | 6.44 | 6.35 | 6.565 | −0.239 |
| 212448_at | AB007899 | NEDD4L | 23327 | 0.38 | 5.971 | 5.83 | 5.754 | 5.52 | 6.27 | 5.996 | −0.264 |
| 212458_at | H97931 | SPRED2 | 200734 | −0.1108 | 5.775 | 5.78 | 5.529 | 5.55 | 5.75 | 5.884 | −0.236 |
| 212461_at | BF793951 | AZIN1 | 51582 | −0.0921 | 9.358 | 9.25 | 9.222 | 9.2 | 9.63 | 9.711 | −0.095 |
| 212463_at | BE379006 | CD59 | 966 | 0.0032 | 7.591 | 7.46 | 7.438 | 7.54 | 7.5 | 7.49 | −0.036 |
| 212466_at | AW138902 | SPRED2 | 200734 | 0.051 | 3.197 | 3.16 | 3.105 | 3.2 | 3.19 | 3.129 | −0.025 |
| 212472_at | BE965029 | MICAL2 | 9645 | 0.0901 | 5.398 | 5.28 | 5.3 | 5.03 | 6.89 | 6.981 | −0.175 |
| 212473_s_at | BE965029 | MICAL2 | 9645 | 0.1689 | 7.891 | 7.75 | 7.875 | 7.71 | 9.29 | 9.403 | −0.028 |
| 212523_s_at | D63480 | KIAA0146 | 23514 | −0.2437 | 4.482 | 4.77 | 4.508 | 4.4 | 3.97 | 4.285 | −0.174 |
| 212551_at | NM_006366 | CAP2 | 10486 | −0.0587 | 6.661 | 6.63 | 6.511 | 6.63 | 6.67 | 6.748 | −0.027 |
| 212554_at | N90755 | CAP2 | 10486 | 0.144 | 6.743 | 6.9 | 6.717 | 6.76 | 6.93 | 6.838 | −0.079 |
| 212574_x_at | AC004528 | C190rf6 | 91304 | 0.0214 | 3.96 | 3.72 | 3.424 | 3.42 | 3.41 | 3.412 | −0.418 |
| 212575_at | BF966155 | C19orf6 | 91304 | −0.1725 | 4.053 | 4.14 | 4.278 | 4.03 | 4.14 | 4.04 | 0.058 |
| 212611_at | AV728526 | DTX4 | 23220 | −0.0026 | 6.365 | 6.62 | 6.345 | 5.92 | 5.79 | 5.942 | −0.362 |
| 212647_at | NM_006270 | RRAS | 6237 | −0.0679 | 7.629 | 7.75 | 7.831 | 7.79 | 7.8 | 7.67 | 0.121 |
| 212718_at | BF797555 | PAPOLA | 10914 | 0.0895 | 10.21 | 10.2 | 10.15 | 10.2 | 10.4 | 10.37 | −0.044 |
| 212720_at | A1670847 | PAPOLA | 10914 | −0.1204 | 6.703 | 6.62 | 6.731 | 6.71 | 6.68 | 6.658 | 0.059 |
| 212722_s_at | AK021780 | JMJD6 | 23210 | 0.1076 | 4.725 | 5.06 | 5.168 | 4.75 | 4.8 | 4.853 | 0.063 |
| 212723_at | 4K021780 | LMLD6 | 23210 | 0.1682 | 7.003 | 7.01 | 6.973 | 7.04 | 7.31 | 7.268 | −8E−04 |
| 212734_x_at | AI186735 | RPL13 | 6137 | −0.0441 | 13.08 | 13.1 | 12.99 | 13.1 | 13 | 13.04 | −0.052 |
| 212777_at | L13857 | SOS1 | 6654 | −0.3525 | 5.611 | 5.33 | 5.41 | 5.64 | 6.12 | 5.647 | 0.057 |
| 212780_at | AA700167 | SOS1 | 6654 | 0.0786 | 5.461 | 5.34 | 5.356 | 5.59 | 5.84 | 5.931 | 0.073 |
| 212816_s_at | BE613178 | CBS | 875 | 0.0159 | 5.586 | 5.36 | 5.465 | 5.11 | 5.89 | 6.115 | −0.182 |
| 212817_at | AK023253 | DNAJB5 | 25822 | −0.2479 | 4.025 | 3.9 | 4.016 | 3.97 | 4.11 | 4.253 | 0.026 |
| 212848_s_at | BG036668 | C9orf3 | 84909 | 0.1426 | 7.71 | 7.81 | 7.772 | 7.72 | 7.62 | 7.612 | −0.013 |
| 212858_at | AL520675 | PAQR4 | 124222 | 0.1029 | 3.416 | 3.61 | 3.554 | 3.41 | 4.12 | 3.808 | −0.03 |
| 212869_x_at | AI721229 | TPT1 | 7178 | 0.0078 | 13.14 | 13.2 | 13.06 | 13.1 | 13.1 | 13.02 | −0.067 |
| 212873_at | BE349017 | HMHA1 | 23526 | 0.1856 | 4.388 | 4.35 | 4.631 | 4.44 | 4.42 | 4.737 | 0.17 |
| 212877_at | AA284075 | KLC1 | 3831 | 0.227 | 6.369 | 6.3 | 6.282 | 6.1 | 6.75 | 6.568 | −0.146 |
| 212878_s_at | AA284075 | KLC1 | 3831 | 0.026 | 7.165 | 7.2 | 7.162 | 7.29 | 7.71 | 7.738 | 0.044 |
| 212898_at | AB007866 | KIAA0406 | 9675 | −0.291 | 8.169 | 8.3 | 8.199 | 8.03 | 7.88 | 7.827 | −0.12 |
| 212910_at | W19873 | THAP11 | 57215 | 0.0316 | 6.803 | 6.86 | 6.654 | 6.81 | 6.78 | 6.626 | −0.101 |
| 212924_s_at | N37057 | LSM4 | 25804 | 0.3237 | 4.558 | 4.61 | 4.623 | 4.87 | 4.82 | 4.775 | 0.162 |
| 212933_x_at | AA961748 | RPL13 | 6137 | −0.0376 | 12.15 | 12.1 | 12.1 | 12.1 | 12 | 11.99 | −0.019 |
| 212944_at | AK024896 | SLCSA3 | 6526 | −0.2681 | 8.74 | 8.64 | 8.509 | 8.49 | 8.14 | 8.16 | −0.189 |
| 212970_at | AI694303 | APBB2 | 323 | 0.036 | 5.097 | 4.74 | 4.744 | 4.96 | 5.52 | 5.765 | −0.065 |
| 212971_at | AI769685 | CARS | 833 | 0.0869 | 10.21 | 10.1 | 10.21 | 10.2 | 10.6 | 10.61 | 0.064 |
| 212972_x_at | AL080130 | APBB2 | 323 | −0.1521 | 4.34 | 4.34 | 4.438 | 4.24 | 4.35 | 4.467 | 4E−06 |
| 212974_at | AI808958 | DENND3 | 22898 | −0.4622 | 3.389 | 3.13 | 3.384 | 3.27 | 3.01 | 3.009 | 0.066 |
| 212975_at | AB020677 | DENND3 | 22898 | −0.0887 | 4.16 | 3.97 | 4.475 | 4.07 | 3.88 | 4.114 | 0.208 |
| 212985_at | BF115739 | APBB2 | 323 | 0.0672 | 5.385 | 5.08 | 4.913 | 5.21 | 5.93 | 5.943 | −0.17 |
| 212992_at | AI935123 | AHNAK2 | 113146 | 0.0024 | 9.086 | 9.08 | 9.08 | 9.35 | 9.15 | 9.168 | 0.13 |
| 213010_at | AI088622 | PRKCDBP | 112464 | 0.0322 | 4.096 | 4.39 | 4.72 | 4.54 | 4.53 | 4.399 | 0.386 |
| 213017_at | AL534702 | ABHD3 | 171586 | −0.1341 | 7.136 | 7.12 | 7.038 | 7.21 | 6.87 | 6.906 | 3E−04 |
| 213043_s_at | AI023317 | MED24 | 9862 | −0.0412 | 5.294 | 4.98 | 5.132 | 5.02 | 4.81 | 5.01 | −0.057 |
| 213072_at | AI928387 | CYHR1 | 50625 | −0.0629 | 4.006 | 4.11 | 3.598 | 3.9 | 4.09 | 4.028 | −0.31 |
| 213076_at | D38169 | ITPKC | 80271 | −0.1039 | 5.026 | 4.83 | 4.851 | 4.75 | 4.9 | 4.836 | −0.127 |
| 213087_s_at | BF690020 | EEF1D | 1936 | 0.6819 | 5.664 | 5.54 | 5.659 | 5.95 | 6.31 | 6.079 | 0.204 |
| 213093_at | AI471375 | PRKCA | 5578 | 0.3155 | 4.265 | 4.05 | 4.287 | 4.56 | 4.39 | 4.475 | 0.267 |
| 213099_at | AB018302 | ANGEL1 | 23357 | −0.1494 | 4.577 | 4.43 | 4.303 | 4.42 | 4.334 | 4.642 | −0.14 |
| 213107_at | R59093 | TNIK | 23043 | −0.0702 | 4.19 | 3.99 | 4.47 | 4.12 | 3.9 | 3.984 | 0.204 |
| 213109_at | N25621 | TNIK | 23043 | −0.2567 | 3.28 | 3.36 | 3.55 | 3.44 | 2.83 | 3.085 | −0.173 |
| 213124_at | BG538800 | ZNF473 | 25888 | 0.0176 | 5.397 | 5.35 | 5.25 | 5.15 | 5.59 | 5.818 | −0.178 |
| 213130_at | AB032967 | 2NF473 | 25888 | 0.2846 | 4.596 | 4.65 | 4.866 | 4.62 | 4.85 | 5.007 | 0.123 |
| 213164_at | AI867198 | SLC5A3 | 6526 | −0.1029 | 8.629 | 8.59 | 8.418 | 8.48 | 7.93 | 7.95 | −0.158 |
| 213167_s_at | BF982927 | SLC5A3 | 6526 | 0.0133 | 3.135 | 3 | 2.909 | 3.06 | 2.8 | 2.658 | −0.085 |
| 213176_s_at | AI910869 | LTBP4 | 8425 | −0.3117 | 4.33 | 4.19 | 4.156 | 4.05 | 3.49 | 3.808 | −0.159 |
| 213252_at | AI739005 | SH3PXD2A | 9644 | −0.1314 | 4.326 | 4.47 | 4.247 | 4.11 | 4.41 | 4.527 | −0.222 |
| 213268_at | Z98884 | CAMTA1 | 23261 | 0.0676 | 3.204 | 3.23 | 3.132 | 3.38 | 3.38 | 3.539 | 0.041 |
| 213288_at | AI761250 | MBOAT2 | 129642 | −0.0892 | 6.138 | 5.91 | 6.038 | 6.15 | 6.01 | 5.859 | 0.071 |
| 213302_at | AL044326 | PFAS | 5198 | 0.0793 | 6.281 | 6.29 | 6.354 | 6.3 | 6.68 | 6.821 | 0.046 |
| 213330_s_at | BE886580 | STIP1 | 10963 | −0.2255 | 7.94 | 8.16 | 8.126 | 7.91 | 7.74 | 7.916 | −0.032 |
| 213333_at | AL520774 | MDH2 | 4191 | 0.0033 | 5.508 | 5.77 | 5.573 | 5.58 | 5.67 | 5.859 | −0.059 |
| 213349_at | AI934469 | TMCC1 | 23023 | 0.4218 | 4.807 | 4.47 | 4.656 | 4.5 | 5.79 | 5.805 | −0.061 |
| 213351_s_at | AB018322 | TMCC1 | 23023 | 0.6026 | 6.293 | 6.4 | 6.378 | 6.47 | 7.57 | 7.481 | 0.079 |
| 213352_at | AB018322 | TMCC1 | 23023 | 0.4588 | 4.231 | 4.13 | 3.977 | 3.7 | 5.47 | 5.319 | −0.342 |
| 213376_at | AI656706 | ZBTB1 | 22890 | 0.0616 | 6.463 | 6.39 | 6.504 | 6.61 | 6.57 | 6.529 | 0.13 |
| 213388_at | H15535 | PDE4DIP | 9659 | 0.2669 | 5.807 | 5.94 | 6.003 | 6.09 | 5.63 | 5.365 | 0.177 |
| 213391_at | AI669947 | DPY19L4 | 286148 | 0.0091 | 7.407 | 7.35 | 7.291 | 7.25 | 6.93 | 6.876 | −0.106 |
| 213397_x_at | AI761728 | RNASE4 | 6038 | 0.208 | 4.378 | 4.81 | 4.401 | 4.69 | 4.14 | 3.469 | −0.051 |
| 213418_at | NM_002155 | HSPA6 | 3310 | 0.1096 | 3.022 | 3.19 | 3.239 | 3.33 | 3.08 | 3.181 | 0.179 |
| 213419_at | U62325 | APBB2 | 323 | 0.3621 | 4.464 | 4.6 | 4.583 | 4.61 | 5.63 | 5.654 | 0.064 |
| 213422_s_at | AW888223 | MXRA8 | 54587 | −0.2379 | 3.106 | 2.89 | 2.866 | 3.03 | 2.99 | 2.82 | −0.052 |
| 213426_s_at | AA15011O | CAV2 | 858 | −0.0265 | 4.098 | 4.38 | 3.945 | 4.02 | 4.45 | 4.443 | −0.255 |
| 213445_at | D63484 | 2C3H3 | 23144 | −0.1401 | 3.905 | 4.11 | 3.928 | 3.97 | 3.99 | 4.142 | −0.055 |
| 213466_at | BE965869 | RAB40C | 57799 | −0.0769 | 3.386 | 3.49 | 3.429 | 3.51 | 3.16 | 3.249 | 0.028 |
| 213481_at | N92920 | S10DA13 | 6284 | 0.2885 | 3.834 | 4 | 3.909 | 3.89 | 4.17 | 4.129 | −0.016 |
| 213487_at | AI762811 | MAP2K2 | 5605 | 0.1351 | 2.781 | 2.7 | 2.931 | 2.77 | 2.94 | 2.797 | 0.107 |
| 213490_s_at | AT762811 | MAP2K2 | 5605 | −0.0634 | 4.718 | 4.83 | 4.697 | 4.84 | 4.73 | 4.862 | −0.007 |
| 213492_at | X06268 | COL2A1 | 1280 | −0.1164 | 3.353 | 3.2 | 3.462 | 3.76 | 2.9 | 2.943 | 0.331 |
| 213509_x_at | AW157619 | CES2 | 8824 | 0.0704 | 7.683 | 7.54 | 7.479 | 7.64 | 7.38 | 7.458 | −0.051 |
| 213535_s_at | AA910614 | UBE2I | 7329 | −0.0844 | 8.614 | 8.59 | 8.78 | 8.75 | 8.62 | 8.576 | 0.164 |
| 213536_s_at | AA910614 | UBE2I | 7329 | 0.4193 | 2.889 | 3.18 | 3.447 | 3.54 | 2.97 | 2.858 | 0.457 |
| 213545_x_at | BE962615 | SNX3 | 8724 | 0.0159 | 9.804 | 9.86 | 9.422 | 9.86 | 9.35 | 9.403 | −0.191 |
| 213551_x_at | AI744229 | PCGF2 | 7703 | 0.0542 | 5.534 | 5.52 | 5.233 | 5.32 | 5.1 | 5.102 | −0.248 |
| 213559_s_at | BF223401 | ZNF467 | 168544 | 0.1798 | 2.756 | 2.92 | 2.945 | 2.77 | 2.81 | 2.733 | 0.022 |
| 213602_s_at | AA401885 | MMP11 | 4320 | −0.037 | 3.476 | 3.25 | 3.166 | 3.2 | 3.32 | 3.364 | −0.179 |
| 213608_s_at | AI220627 | SRRD | 402055 | −0.0081 | 6.143 | 6.08 | 6.218 | 6.13 | 6.73 | 6.83 | 0.066 |
| 213636_at | AB028968 | KIAA1045 | 23349 | −0.2844 | 2.959 | 2.95 | 2.817 | 2.8 | 2.96 | 2.792 | −0.146 |
| 213549_at | AA524053 | SFRS7 | 6432 | 0.0444 | 9.152 | 9.03 | 9.247 | 9.13 | 9.32 | 9.245 | 0.099 |
| 213656_s_at | BF593594 | KLC1 | 3831 | 0.2473 | 7.885 | 7.92 | 8.108 | 8.13 | 8.31 | 8.378 | 0.217 |
| 213681_at | AW512817 | CYHR1 | 50626 | −0.0103 | 4.02 | 3.82 | 3.968 | 3.85 | 3.95 | 3.905 | −0.011 |
| 213688_at | N25325 | CALM1 /// CALM2 /// | 801 /// 805 /// 808 | 0.0383 | 3.384 | 3.4 | 3.233 | 3.5 | 3.4 | 3.684 | −0.025 |
| | | CALM3 | | | | | | | | | |
| 213708_s_at | N40555 | MLX | 6945 | 0.1359 | 8.344 | 8.29 | 8.299 | 8.36 | 8.78 | 8.817 | 0.012 |
| 213741_s_at | BF575685 | KPNA1 | 3836 | −0.0886 | 6.627 | 6.61 | 6.567 | 6.71 | 6.54 | 6.674 | 0.022 |
| 213849_s_at | AA974416 | PPP2R2B | 5521 | −0.0496 | 5.058 | 4.79 | 5.097 | 4.65 | 5.91 | 5.809 | −0.048 |
| 213858_at | BE350026 | ZNF250 | 58500 | 0.0847 | 4.078 | 3.96 | 3.819 | 3.97 | 4.03 | 4.128 | −0.126 |
| 213871_s_at | AA523444 | C6orf108 | 10591 | 0.1896 | 3.266 | 3.22 | 3.1 | 2.9 | 2.86 | 3.177 | −0.243 |
| 213889_at | AI742901 | PIGL | 9487 | 0.3712 | 4.859 | 4.98 | 4.454 | 4.88 | 4.92 | 4.572 | −0.251 |
| 213910_at | AW770896 | IGFBP7 | 3490 | −0.102 | 4.636 | 4.38 | 4.159 | 4.23 | 4.38 | 4.068 | −0.313 |
| 213917_at | BE465829 | PAX8 | 7849 | −0.2121 | 3.142 | 2.84 | 2.97 | 2.93 | 2.82 | 3.117 | −0.041 |
| 213927_at | AV753204 | MAP3K9 | 4293 | 0.1817 | 5.115 | 5.07 | 5.049 | 4.98 | 5.44 | 5.357 | −0.076 |
| 213941_x_at | AI970731 | RPS7 | 6201 | 0.0387 | 12.43 | 12.4 | 12.4 | 12.4 | 12.5 | 12.36 | −0.016 |
| 213942_at | AL134303 | MEGF6 | 1953 | 0.025 | 4.145 | 3.94 | 3.809 | 3.97 | 3.86 | 4.076 | −0.151 |
| 213969_x_at | BF683426 | RPL29 /// RPL29P4 | 387101 /// 6159 | −0.0698 | 12.83 | 32.8 | 12.8 | 12.8 | 12.7 | 12.69 | −0.01 |
| 213982_s_at | BG107203 | RABGAP1L | 9910 | 0.0522 | 5.141 | 5.13 | 4.805 | 5.04 | 5.1 | 5.013 | −0.211 |
| 213985_s_at | H45660 | C19orf6 | 91304 | 0.1455 | 3.296 | 3.16 | 3.348 | 3.14 | 3.27 | 3.203 | 0.013 |
| 213986_s_at | AI805266 | C19orf6 | 91304 | −0.2016 | 4.569 | 4.92 | 4.953 | 4.88 | 4.56 | 4.754 | 0.172 |
| 214026_s_at | AI860246 | SPRED2 | 200734 | 0.0905 | 2.815 | 2.8 | 3.075 | 2.87 | 2.82 | 2.825 | 0.162 |
| 214040_s_at | BE675337 | GSN | 2934 | −0.3644 | 4.072 | 4.71 | 4.443 | 4.35 | 4.02 | 4.087 | 0.O05 |
| 214047_s_at | AI913365 | MBD4 | 8930 | 0.0441 | 7.385 | 7.4 | 7.459 | 7.46 | 7.69 | 7.762 | 0.063 |
| 214048_at | AI953365 | MBD4 | 8930 | 0.0841 | 4.906 | 4.9 | 4.856 | 5.07 | 4.53 | 4.812 | 0.059 |
| 254061_at | AI017564 | WDR67 | 93594 | 0.1002 | 5.546 | 5.54 | 5.659 | 5.72 | 5.65 | 5.645 | 0.142 |
| 214080_x_at | AI815793 | PRKCSH | 5589 | −0.0931 | 7.446 | 7.59 | 7.584 | 7.49 | 7.59 | 7.598 | 0.019 |
| 214099_s_at | AK001619 | PDE4DIP | 9659 | 0.1095 | 3.536 | 3.65 | 3.816 | 3.75 | 3.64 | 3.872 | 0.194 |
| 214129_at | AI821791 | PDE4DIP | 9659 | 0.2845 | 5.653 | 5.52 | 5.069 | 5.42 | 5.84 | 5.672 | −0.341 |
| 214130_s_at | AI821791 | PDE4DIP | 9659 | −0.0707 | 3.408 | 3.49 | 3.616 | 3.52 | 3.82 | 3.663 | 0.117 |
| 214134_at | BF939689 | C2orf55 | 343990 | 0.0881 | 2.958 | 2.87 | 3.153 | 2.89 | 2.87 | 2.913 | 0.109 |
| 214141_x_at | BF033354 | SFRS7 | 6432 | 0.1012 | 9.679 | 9.68 | 9.68 | 9.61 | 10.2 | 10.21 | −0.034 |
| 214164_x_at | BF752277 | CA12 | 771 | 0.0309 | 7.104 | 6.94 | 7.229 | 7.41 | 7.26 | 6.476 | 0.297 |
| 214177_s_at | AI935162 | PBXIP1 | 57326 | 0.045 | 4.96 | 5.14 | 5.222 | 4.8 | 4.96 | 4.727 | −0.038 |
| 214239_x_at | AI560455 | PCGF2 | 7703 | 0.1364 | 7.182 | 7.21 | 6.94 | 7.12 | 6.89 | 6.99 | −0.163 |
| 214310_s_at | AI767884 | ZFPL1 | 7542 | −0.0225 | 3.678 | 3.82 | 3.981 | 3.61 | 3.76 | 3.949 | 0.047 |
| 214311_at | AI767884 | ZFPL1 | 7542 | −0.1898 | 2.938 | 2.94 | 3.053 | 2.93 | 3 | 2.914 | 0.051 |
| 214327_x_at | AI888178 | TPT1 | 7178 | 0.0029 | 12.47 | 12.5 | 12.47 | 12.5 | 12.4 | 12.33 | 0.019 |
| 214328_s_at | R01140 | HSP90AA1 | 3320 | −0.1491 | 11.93 | 11.9 | 11.91 | 11.9 | 11.6 | 11.63 | −0.025 |
| 214335_at | AI669349 | RPL18 | 6141 | −0.1456 | 3.466 | 3.44 | 3.261 | 3.69 | 3.44 | 3.357 | 0.021 |
| 214336_s_at | AI621079 | COPA | 1314 | −0.4409 | 7.139 | 7.26 | 7.214 | 7.33 | 6.54 | 6.769 | 0.07 |
| 214337_at | AI621079 | COPA | 1314 | −0.0261 | 3.076 | 2.96 | 3.142 | 3.35 | 2.96 | 3.03 | 0.23 |
| 214338_at | AL050381 | DNAJB12 | 54788 | 0.1178 | 4.55 | 4.49 | 4.355 | 4.34 | 4.1 | 4.295 | −0.176 |
| 214351_x_at | AA789278 | RPL13 | 6137 | −0.0634 | 12.24 | 12.2 | 12.19 | 12.2 | 12.2 | 12.15 | 0.007 |
| 214359_s_at | AI218219 | HSP90AB1 | 3326 | −0.5467 | 10.23 | 10.3 | 10.23 | 10.2 | 9.38 | 9.493 | −0.033 |
| 214391_x_at | AI762344 | PTGER1 | 5731 | 0.2532 | 3.281 | 3.54 | 3.576 | 3.31 | 3.51 | 3.732 | 0.031 |
| 214394_x_at | AI613383 | EEF1D | 1936 | 0.0934 | 11.87 | 11.9 | 11.82 | 11.8 | 11.9 | 11.91 | −0.064 |
| 214395_x_at | AI335509 | EEF1D | 1936 | 0.2292 | 6.215 | 6.17 | 6.296 | 6.18 | 6.72 | 6.447 | 0.048 |
| 214430_at | NM_000169 | GLA | 2717 | 0.1721 | 7.19 | 7.17 | 7.178 | 7.23 | 6.99 | 7.047 | 0.024 |
| 214482_at | NM_006977 | ZBTB25 | 7597 | 0.1506 | 3.932 | 4.18 | 4.091 | 4.15 | 4.08 | 4.367 | 0.066 |
| 214494_s_at | NM_005200 | SPG7 | 6687 | −0.0692 | 7.502 | 7.47 | 7.575 | 7.25 | 7.14 | 7.05 | −0.071 |
| 214516_at | NM_003544 | HIST1H4A /// | 121504 /// 554313 | −0.1405 | 2.852 | 2.74 | 2.824 | 3.02 | 2.7 | 3.054 | 0.126 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 214528_s_at | NM_013951 | PAX8 | 7849 | 0.1543 | 2.615 | 2.44 | 2.588 | 2.52 | 2.6 | 2.802 | 0.023 |
| 214536_at | NM_020427 | SLURP1 | 57152 | −0.2251 | 2.896 | 2.88 | 2.72 | 2.71 | 2.91 | 3.038 | −0.17 |
| 214544_s_at | NM_003825 | SNAP23 | 8773 | −0.2784 | 4.648 | 4.76 | 4.722 | 4.17 | 4.29 | 3.913 | −0.262 |
| 214550_s_at | AFI45029 | TNPO3 | 23534 | −0.1517 | 6.617 | 6.61 | 6.606 | 6.7 | 6.65 | 6.615 | 0.043 |
| 214600_at | AW771935 | TFAD1 | 7003 | −6.0267 | 5.953 | 6.03 | 5.937 | 5.9 | 6.08 | 5.885 | −0.08 |
| 234606_s_at | AJ000098 | EYA1 | 2138 | 0.1103 | 3.016 | 3.07 | 2.891 | 2.86 | 3.03 | 2.993 | −0.164 |
| 214634_at | AL523073 | HIST1H4A /// | 121504 /// 554313 | 0.1167 | 3.325 | 3.37 | 3.319 | 3.34 | 3.48 | 3.377 | −0.018 |
| | | HIST1H4B /// | /// 8294 /// 8359 /// | | | | | | | | |
| | | HIST1H4C /// | 8360 /// 8361 /// | | | | | | | | |
| | | HIST1H4D /// | 8362 /// 8363 /// | | | | | | | | |
| | | HIST1H4E /// | 8364 /// 8365 /// | | | | | | | | |
| | | HIST1H4F /// | 8366 /// 8367 /// | | | | | | | | |
| | | HIST1H4H /// | 8368 /// 8370 | | | | | | | | |
| | | HIST1H4I /// | | | | | | | | | |
| | | HIST1H4J /// | | | | | | | | | |
| | | HIST1H4K /// | | | | | | | | | |
| | | HIST1H4L /// | | | | | | | | | |
| | | HIST2H4A /// | | | | | | | | | |
| | | HIST2H4B /// | | | | | | | | | |
| | | HIST4H4 | | | | | | | | | |
| 214692_s_at | AL041139 | JRK | 8629 | 0.0892 | 5.323 | 5.51 | 5.383 | 5.4 | 5.18 | 5.076 | −0.022 |
| 214721_x_at | AL162074 | CDC42EP4 | 23580 | 0.055 | 4.317 | 4.27 | 4.123 | 4.12 | 4.3 | 4.366 | −0.17 |
| 214743_at | BE046521 | CUX1 | 1523 | 0.2274 | 8.077 | 7.97 | 8.031 | 7.89 | 8.01 | 8.053 | −0.062 |
| 214746_s_at | BE549732 | ZNF467 | 168544 | −0.0958 | 3.151 | 3.49 | 3.068 | 3.18 | 3.21 | 3.167 | −0.195 |
| 214748_at | US0529 | N4BP2L2 | 10443 | −0.2023 | 4.671 | 4.27 | 4.844 | 4.86 | 4.85 | 4.487 | 0.38 |
| 214753_3t | AW084068 | N4BP2L2 | 10443 | −0.0943 | 7.117 | 7.17 | 7.304 | 7.18 | 7.4 | 7.258 | 0.096 |
| 214760_at | AL049942 | 2NF337 | 26152 | 0.037 | 6.369 | 6.32 | 6.232 | 6.25 | 6.48 | 6.624 | −0.103 |
| 214818_at | AF007146 | CCDC57 | 284001 | −0.1098 | 3.665 | 3.62 | 3.566 | 3.49 | 3.51 | 3.686 | −0.116 |
| 214827_at | AL031680 | PARD6B | 84612 | 0.1099 | 2.946 | 2.84 | 2.712 | 2.91 | 2.85 | 3.072 | −0.081 |
| 214882_s_at | BG254869 | SFRS2 | 6427 | 0.0498 | 9.786 | 9.66 | 9.755 | 9.5S | 9.85 | 9.946 | −0.065 |
| 214894_x_at | AK023285 | MACF1 | 23499 | −0.1036 | 6.833 | 6.77 | 6.726 | 6.67 | 6.63 | 6.825 | −0.1 |
| 214925_s_at | AK026484 | SPTAN1 | 6709 | −0.2827 | 3.984 | 4.18 | 4.253 | 4.01 | 4.21 | 3.936 | −0.051 |
| 214926_at | AK026484 | SPTAN1 | 6709 | −0.1358 | 2.985 | 2.77 | 2.666 | 2.91 | 2.79 | 2.957 | −0.091 |
| 214953_s_at | X06989 | APP | 351 | 0.0651 | 9.042 | 8.75 | 8.691 | 8.45 | 8.61 | 8.046 | −0.327 |
| 214969_at | AF2S1442 | MAP3K9 | 4293 | −0.1259 | 3.026 | 2.87 | 3.041 | 3.09 | 2.78 | 3.051 | 0.114 |
| 214976_at | AI554467 | RPL13 | 6137 | −0.108 | 3.825 | 3.94 | 3.786 | 3.91 | 3.87 | 3.822 | −0.037 |
| 215005_at | AV723666 | NECAB2 | 54550 | 0.0964 | 3.685 | 3.75 | 3.585 | 3.68 | 3.52 | 3.6 | −0.087 |
| 215046_at | AL133053 | C2orf67 | 151050 | 0.0411 | 2.757 | 2.85 | 2.955 | 2.96 | 2.96 | 2.921 | 0.153 |
| 215069_at | AK025065 | NMT2 | 9397 | 0.1555 | 3.294 | 3.12 | 3.086 | 3.39 | 3.33 | 3.148 | 0.032 |
| 215092_s_at | AJ005683 | NFAT5 | 10725 | −0.1955 | 6.287 | 6.07 | 5.867 | 6.31 | 5.94 | 6.052 | −0.09 |
| 215157_x_at | AI734929 | PABPC1 | 26986 | 0.0154 | 12.63 | 12.6 | 12.53 | 12.6 | 12.6 | 12.6 | 0.019 |
| 215184_at | AK026801 | DAPK2 | 23604 | 0.0169 | 3.515 | 3.62 | 3.305 | 3.64 | 3.4 | 3.507 | −0.095 |
| 215194_at | AF035594 | PRKCA | 5578 | 0.1818 | 3.325 | 2.8 | 2.869 | 3 | 3.04 | 3.054 | −0.026 |
| 215195_at | AF035594 | PRKCA | 5578 | 0.1126 | 3.703 | 3.69 | 3.549 | 3.75 | 3.72 | 3.789 | −0.05 |
| 215205_x_at | S83390 | NCOR2 | 9612 | 0.1042 | 2.941 | 2.78 | 2.95 | 3.13 | 2.98 | 2.86 | 0.179 |
| 215222_x_at | AK023406 | MACF1 | 23499 | −0.1615 | 6.618 | 6.51 | 6.359 | 6.5 | 6.31 | 6.46 | −0.133 |
| 215231_at | AU144309 | PRKAG2 | 51422 | −0.0582 | 3.913 | 3.48 | 3.653 | 3.34 | 3.97 | 3.465 | −0.202 |
| 215233_at | AA351360 | JMJD6 | 23210 | −0.1574 | 3.444 | 3.41 | 3.523 | 3.22 | 3.36 | 3.4 | −0.056 |
| 215235_at | AL110273 | SPTAN1 | 6709 | 0.2549 | 5.673 | 5.48 | 5.65 | 5.54 | 6.17 | 6.235 | 0.021 |
| 215240_at | AI189839 | ITGB3 | 3690 | 0.0007 | 2.663 | 2.7 | 2.657 | 2.85 | 2.9 | 2.811 | 0.073 |
| 215270_at | U94354 | LFNG | 3955 | −0.0947 | 2.852 | 2.76 | 2.988 | 3.09 | 2.96 | 2.892 | 0.233 |
| 215337_at | AK022508 | MED24 | 9862 | 0.0525 | 3.055 | 3.19 | 3.077 | 3.13 | 3.35 | 3.028 | −0.021 |
| 215342_s_at | AB019490 | RABGAP1L | 9910 | 0.0366 | 4.88 | 4.89 | 5.093 | 5.15 | 4.76 | 4.843 | 0.237 |
| 215374_at | AK024849 | PAPOLA | 10914 | −0.1382 | 3.48 | 3.56 | 3.319 | 3.44 | 3.32 | 3.341 | −114 |
| 215377_at | AK024129 | CTBP2 | 1488 | 0.1724 | 3.925 | 3.95 | 3.968 | 3.94 | 3.79 | 3.803 | 0.013 |
| 215548_s_at | AB020724 | SCFD1 | 23256 | −0.0901 | 8.127 | 8.08 | 8.038 | 8.26 | 8.22 | 8.476 | 0.047 |
| 215575_at | AU157078 | PDE4DIP | 9659 | 0.2403 | 3.012 | 2.93 | 3.042 | 3.06 | 2.94 | 2.92 | 0.079 |
| 215584_at | AK022679 | HECW1 | 23072 | 0.1695 | 3.19 | 3.26 | 3.517 | 3.11 | 3.34 | 3.365 | 0.089 |
| 215517_at | AU145711 | LOC26010 | 26010 | 0.0549 | 2.895 | 2.83 | 2.775 | 2.99 | 2.67 | 2.6 | 0.025 |
| 215631_s_at | AL0S0G08 | BRMS1 | 25855 | −0.011 | 6.174 | 6.12 | 6.548 | 6.19 | 6.42 | 6.474 | 0.223 |
| 215688_at | AL359931 | RASGRF1 | 5923 | −0.2743 | 3.284 | 3.42 | 3.599 | 3.39 | 3.24 | 3.236 | 0.142 |
| 215728_at | AL031848 | ACOT7 | 11332 | −0.0998 | 5.153 | 5.11 | 5.143 | 4.81 | 5.48 | 5.561 | −0.153 |
| 215732_s_at | AK023924 | DTX2 /// | 100134197 // | 0.0336 | 4.445 | 4.77 | 5.041 | 4.66 | 4.33 | 4.66 | 0.24 |
| | | LOC100134197 | 113878 | | | | | | | | |
| 215743_at | AL134483 | NMT2 | 9397 | −0.0801 | 3.202 | 3.27 | 3.23 | 3.11 | 3.34 | 2.959 | −0.064 |
| 215852_x_at | AK022023 | C20orftL17 | 140710 | 0.0522 | 3.395 | 3.76 | 3.283 | 3.4 | 3.52 | 3.117 | −0.233 |
| 215867_x_at | AL050025 | CA12 | 771 | 0.0409 | 7.035 | 6.89 | 7.042 | 7.29 | 7.37 | 6.324 | 0.206 |
| 215912_at | AA758795 | GNAO1 | 2775 | −0.0239 | 3.286 | 3.36 | 3.421 | 3.33 | 3.39 | 3.366 | 0.054 |
| 215938_s_at | AK001290 | PLA2G6 | 8398 | −0.0233 | 3.329 | 3.2 | 3.351 | 3.23 | 3.13 | 3.161 | 0.024 |
| 215980_s_at | AF052128 | IGHMBP2 | 3508 | 0.2051 | 3.844 | 3.81 | 3.646 | 3.83 | 3.69 | 3.681 | −0.091 |
| 215991_s_at | AU121504 | KIAA0090 | 23065 | 0.1828 | 2.932 | 2.92 | 2.851 | 3.11 | 3.03 | 2.944 | 0.057 |
| 216105_x_at | X86428 | PPP2R4 | 5524 | 0.0573 | 4.964 | 4.98 | 4.924 | 4.99 | 4.9 | 4.846 | −0.018 |
| 216261_at | AI151479 | ITGB3 | 3690 | −0.057 | 2.986 | 2.91 | 2.954 | 2.81 | 2.99 | 2.975 | −0.068 |
| 216309_x_at | AF072467 | JPX | 8629 | −0.1124 | 5.964 | 5.9 | 6.119 | 5.89 | 5.59 | 5.547 | 0.07 |
| 216364_s_at | AJ001550 | AFF2 | 2334 | −0.0192 | 2.72 | 2.72 | 2.565 | 2.77 | 2.71 | 2.723 | −0.094 |
| 216382_s_at | U80756 | MLL2 | 8085 | −0.19 | 3.647 | 3.52 | 3.654 | 3.55 | 3.42 | 3.386 | 0.02 |
| 216407_at | U25801 | VAC14 | 55697 | 0.3651 | 3.907 | 3.74 | 3.954 | 4 | 4.07 | 4.11 | 0.156 |
| 216501_at | U25801 | VAC14 | 55697 | −0.071 | 2.901 | 2.83 | 2.802 | 2.98 | 2.63 | 2.91 | 0.027 |
| 216520_s_at | AF072098 | TPT1 | 7178 | −0.0507 | 13.02 | 13 | 13.02 | 13 | 13 | 12.92 | −0.037 |
| 216533_at | AL122056 | PCCA | 5095 | −0.1755 | 2.584 | 2.55 | 2.682 | 2.5 | 2.48 | 2.545 | 0.021 |
| 216570_x_at | AL096829 | LOC100131713 /// | 100131713 /// | −0.2619 | 10.63 | 10.6 | 10.47 | 10.5 | 10 | 10.21 | −0.145 |
| | | LOC283412 /// | 283412 /// 284064 | | | | | | | | |
| | | LOC284064 /// | /// 387101 /// | | | | | | | | |
| | | LOC391019 /// | 391019 /// 6159 /// | | | | | | | | |
| | | LOC643531 /// | 643531 /// 647285 | | | | | | | | |
| | | LOC647285 /// | /// 728820 | | | | | | | | |
| | | LOC728820 /// RPL29 | | | | | | | | | |
| | | /// RPL29P4 | | | | | | | | | |
| 216624_s_at | Z69744 | MLL | 4297 | −0.0095 | 3.371 | 3.05 | 3.304 | 3.03 | 3.05 | 2.866 | −0.042 |
| 216678_at | AK000773 | IFT122 | 55764 | −0.0861 | 4.18 | 3.92 | 4.024 | 4.07 | 4.01 | 4.135 | −0.001 |
| 216697_at | AL161955 | TRIO | 7204 | −0.1116 | 3.269 | 3.41 | 2.949 | 3.09 | 3.28 | 3.355 | −0.317 |
| 216700_at | AL161955 | TRIO | 7204 | −0.0135 | 3.182 | 3.35 | 3.222 | 3.18 | 3.2 | 3.169 | −0.063 |
| 216747_at | AK024871 | APBB2 | 323 | −0.204 | 3.108 | 3.37 | 3.224 | 3.11 | 3.36 | 3.449 | −0.071 |
| 216750_at | AK024871 | APBB2 | 323 | −0.5452 | 3.519 | 3.1 | 3.078 | 2.87 | 3.2 | 2.927 | −0.336 |
| 216845_x_at | U80756 | MLL2 | 8085 | −0.2494 | 3.552 | 3.78 | 3.795 | 3.42 | 3.37 | 3.389 | −0.056 |
| 216867_s_at | X03795 | PDGFA | 5154 | 0.2831 | 5.615 | 5.69 | 5.797 | 5.82 | 6.29 | 6.217 | 0.154 |
| 216880_at | Y15571 | RAD51L1 | 5890 | 0.1966 | 3.709 | 3.55 | 3.677 | 3.3 | 3.68 | 3.638 | −0.141 |
| 216944_x_at | U23850 | ITPR1 | 3708 | −0.0126 | 3.036 | 3.08 | 3.073 | 3.01 | 2.75 | 3.218 | −0.018 |
| 216952_s_at | M94363 | LMNB2 | 84823 | −0.0114 | 4.803 | 4.91 | 4.693 | 4.73 | 4.73 | 4.937 | −0.148 |
| 216971_s_at | 254367 | PLEC1 | 5339 | −0.1435 | 4.366 | 4.63 | 4.508 | 4.53 | 4.51 | 4.47 | 0.023 |
| 216988_s_at | L48722 | PTP4A2 | 8073 | 0.0332 | 8.036 | 7.87 | 8.014 | 8.08 | 8.35 | 8.325 | 0.095 |
| 217005_at | M28219 | LDLR | 3949 | 0.073 | 3.477 | 3.32 | 3.418 | 3 | 3.25 | 3.223 | −0.191 |
| 217025_s_at | AL110225 | DBN1 | 1627 | 0.1676 | 3.599 | 3.88 | 3.642 | 3.71 | 3.95 | 3.868 | −0.06 |
| 217103_at | M28219 | LDLR | 3949 | 0.1326 | 2.964 | 2.99 | 3.305 | 3.06 | 2.92 | 3.15 | 0.285 |
| 217118_s_at | AK025608 | C22orf9 | 23313 | 0.0986 | 7.445 | 7.69 | 7.404 | 7.41 | 7.92 | 7.858 | −0.159 |
| 217124_at | AL136792 | IQCE | 23288 | 0.0039 | 3.08 | 3.04 | 3.13 | 3.21 | 3.17 | 3.252 | 0.11 |
| 217144_at | X04801 | LOC648390 /// | 6233 /// 648390 /// | −0.551 | 5.954 | 6.24 | 5.788 | 6.03 | 4.92 | 5.231 | −0.191 |
| | | RPS27A /// UBB /// | 7314 /// 7316 | | | | | | | | |
| | | UBC | | | | | | | | | |
| 217146_at | AF072468 | JRK | 8629 | 0.0164 | 2.882 | 3.24 | 3.062 | 2.97 | 2.83 | 3.066 | −0.045 |
| 217173_s_at | S70123 | LDLR | 3949 | −0.1006 | 5.104 | 5.31 | 6.752 | 5.05 | 5.43 | 5.883 | 0.345 |
| 217174_s_at | AL078616 | APC2 | 10297 | −0.0819 | 3.111 | 2.99 | 3.159 | 3.04 | 2.94 | 3.095 | 0.053 |
| 217183_at | S70123 | LDLR | 3949 | −0.0188 | 3.287 | 3.22 | 3.211 | 3.1 | 3.29 | 3.053 | −0.101 |
| 217262_s_at | BC000059 | CELSR1 | 9620 | 0.1168 | 3.062 | 2.91 | 3 | 2.86 | 2.19 | 2.913 | −0.056 |
| 217299_s_at | AK001017 | NBN | 4683 | −0.019 | 5.245 | 5.32 | 5.306 | 5.28 | 5.38 | 5.24 | 0.013 |
| 217356_s_at | S81916 | PGR1 | 5230 | −0.2518 | 10.11 | 10.2 | 10.2 | 10.2 | 9.78 | 9.817 | 0.049 |
| 217383_at | S81916 | PGK1 | 5230 | −0.0547 | 4.133 | 4.13 | 4.312 | 4.37 | 4.55 | 4.131 | 0.21 |
| 217404_s_at | X16468 | COL2A1 | 1280 | 0.0923 | 3.068 | 3.18 | 2.991 | 2.92 | 2.85 | 2.784 | −0.172 |
| 217432_s_at | AF179281 | IDS | 3423 | −0.0571 | 3.649 | 4.03 | 3.887 | 4 | 3.69 | 3.973 | 0.103 |
| 217466_x_at | L48784 | RP52 | 6187 | −0.1497 | 10.28 | 10.2 | 10.15 | 10.2 | 10 | 10.02 | −0.089 |
| 217489_s_at | S72848 | IL6R | 3570 | −0.0524 | 3.038 | 2.66 | 2.936 | 3.19 | 3.11 | 2.794 | 0.211 |
| 217500_at | R27378 | TIAL1 | 7073 | −0.1197 | 3.202 | 3.22 | 2.92 | 3.12 | 2.91 | 2.996 | −0.189 |
| 217508_s_at | BE783279 | C18orf25 | 147339 | 0.1199 | 3.913 | 3.88 | 3.773 | 4.02 | 4.29 | 4.379 | −0.002 |
| 217539_at | W28849 | C18orf25 | 147339 | 0.1061 | 2.744 | 2.62 | 2.734 | 2.81 | 2.78 | 2.804 | 0.092 |
| 217608_at | AW408767 | SFRS12IP1 | 285672 | 0.1559 | 5.361 | 5.4 | 5.185 | 5.23 | 5.28 | 5.323 | −0.169 |
| 217618_x_at | AW007988 | HUS1 | 3364 | −0.0778 | 4.411 | 4.48 | 4.611 | 4.44 | 4.78 | 5.137 | 0.079 |
| 217622_at | AA018187 | RHBDD3 | 25807 | −0.621 | 4.929 | 5.14 | 4.826 | 4.78 | 5.01 | 4.99 | −0.23 |
| 217635_s_at | AA769006 | POLG | 5428 | 0.116 | 5.219 | 5.14 | 5.166 | 5.3 | 5.12 | 5.203 | 0.055 |
| 217636_at | AA769006 | POLG | 5428 | 0.0684 | 2.955 | 2.87 | 2.866 | 2.86 | 2.79 | 2.874 | −0.048 |
| 217669_s_at | AW451230 | AKAP6 | 9472 | 0.0462 | 3.255 | 3.08 | 3.342 | 3.06 | 3.37 | 3.419 | 0.034 |
| 217686_at | BF222916 | PTPN1 | 5770 | −0.1199 | 3.427 | 3.65 | 3.444 | 3.5 | 3.57 | 3.551 | −0.068 |
| 217689_at | BG109555 | PTPN1 | 5770 | −0.0334 | 2.97 | 2.85 | 3.178 | 2.63 | 2.89 | 2.954 | −0.009 |
| 217722_s_at | NM_016645 | NGRN | 51335 | −0.1299 | 10.22 | 10.3 | 10.23 | 10.2 | 10.3 | 10.32 | −0.021 |
| 217745_s_at | NM_025146 | NAT13 | 80218 | 0.0163 | 9.177 | 9.18 | 9.089 | 9.15 | 9.27 | 9.256 | −0.061 |
| 217752_s_at | NM_018235 | CNDP2 | 55748 | 0.1311 | 9.231 | 9.18 | 9.094 | 9.15 | 9.42 | 9.477 | −0.087 |
| 217756_x_at | NM_005770 | SERF2 | 10169 | −0.1423 | 9.791 | 9.88 | 9.859 | 9.86 | 9.73 | 9.794 | 0.021 |
| 217774_s_at | NM_016404 | HSPC152 | 51504 | −0.1028 | 10.73 | 10.8 | 10.72 | 10.8 | 10.5 | 10.57 | −0.044 |
| 217779_s_at | NM_017761 | LOC100132235 /// | 100132235 /// 55629 | 0.1529 | 9.301 | 9.36 | 9.276 | 9.39 | 9.4 | 9.339 | 7E−04 |
| | | PNRC2 | | | | | | | | | |
| 217786_at | NM_006109 | PRMT5 | 10419 | −0.0067 | 8.241 | 8.29 | 8.41 | 8.36 | 8.69 | 8.857 | 0.12 |
| 217793_at | AL575337 | RAB11B | 9230 | 0.1907 | 3.789 | 3.62 | 3.68 | 3.87 | 3.72 | 3.874 | 0.073 |
| 217830_s_at | AL109658 | NSFL1C | 55968 | 0.1308 | 6.888 | 6.91 | 6.984 | 6.91 | 6.84 | 6.804 | 0.049 |
| 217831_s_at | NM_016143 | NSFL1C | 55968 | 0.1266 | 7.046 | 7.08 | 6.892 | 6.94 | 6.96 | 7.017 | −0.143 |
| 217868_s_at | NM_016025 | METTL9 | 51108 | 0.0433 | 9.356 | 9.42 | 9.319 | 9.43 | 9.17 | 9.079 | −0.012 |
| 217875_s_at | NM_020182 | PMEPA1 | 56937 | −0.0103 | 6.945 | 6.8 | 6.986 | 7 | 7.92 | 7.941 | 0.122 |
| 217903_at | NM_013403 | STRN4 | 29888 | 0.291 | 4.516 | 4.74 | 5.031 | 4.74 | 5.16 | 4.878 | 0.258 |
| 217907_at | NM_014161 | MRPL18 | 29074 | −0.1013 | 9.748 | 9.74 | 9.88 | 9.81 | 9.77 | 9.736 | 0.105 |
| 217909_s_at | BF056105 | MLX | 6945 | −0.1247 | 7.227 | 7.46 | 7.401 | 7.33 | 7.42 | 7.607 | 0.022 |
| 217910_x_at | NM_013383 | MLX | 6945 | 0.0991 | 7.891 | 7.85 | 7.849 | 7.72 | 8.05 | 8.322 | −0.084 |
| 217911_s_at | NM_004281 | BAG3 | 9531 | 0.0351 | 8.768 | 8.82 | 8.671 | 8.57 | 8.24 | 8.17 | 0.174 |
| 217924_at | AL523965 | C6orf106 | 64771 | 0.1183 | 3.651 | 3.81 | 3.439 | 3.74 | 3.68 | 4.067 | −0.143 |
| 217925_s_at | NM_022758 | C6orf106 | 64771 | 0.1508 | 5.154 | 5.24 | 5.344 | 5.41 | 5.17 | 5.553 | 0.18 |
| 217943_s_at | NM_018067 | MAP7D1 | 55700 | 0.2198 | 5.877 | 5.95 | 5.55 | 5.63 | 6.24 | 6.296 | −0.327 |
| 217950_at | NM_015953 | NOSIP | 51070 | −0.0471 | 7.033 | 7.02 | 7.195 | 6.99 | 7.22 | 7.293 | 0.067 |
| 217969_at | NM_013265 | C11orf2 | 738 | 0.1902 | 7.94 | 7.76 | 7.958 | 7.73 | 7.91 | 7.884 | −0.005 |
| 217980_s_at | NM_017840 | MRPL16 | 54948 | 0.0139 | 7.64 | 7.75 | 7.614 | 7.69 | 7.92 | 7.838 | −0.042 |
| 218016_s_at | NM_018119 | POLR3E | 55718 | −0.0562 | 6.035 | 5.97 | 6.133 | 5.97 | 6.35 | 6.415 | 0.05 |
| 218018_at | AW449022 | PDXK | 8566 | −0.1592 | 7.034 | 7.1 | 7.136 | 7.21 | 7.44 | 7.553 | 0.106 |
| 218019_s_at | NM_021941 | PDXK | 8566 | 0.2161 | 6.86 | 6.88 | 6.867 | 6.85 | 6.48 | 6.515 | −0.008 |
| 218022_at | NM_016440 | VRX3 | 51231 | 3.3197 | 6.509 | 6.89 | 6.605 | 6.74 | 6.56 | 6.854 | −0.025 |
| 218023_s_at | NM_016605 | FAM53C | 51307 | 0.0959 | 5.783 | 6.65 | 5.915 | 5.95 | 5.83 | 5.726 | 0.216 |
| 218062_x_at | NM_012121 | CDC42EP4 | 23580 | −0.001 | 4.834 | 4.62 | 4.818 | 4.67 | 4.85 | 5.146 | 0.018 |
| 218063_s_at | AF099664 | CDC42EP4 | 23580 | −0.2337 | 2.903 | 2.95 | 2.856 | 2.9 | 2.98 | 2.872 | −0.049 |
| 218074_at | NM_016062 | FAM96B | 51647 | 0.0165 | 9.339 | 9.33 | 9.361 | 9.34 | 9.5 | 9.454 | 0.016 |
| 218099_at | NM_018469 | TEX2 | 55852 | 0.1381 | 6.831 | 6.78 | 6.785 | 6.73 | 6.92 | 6.844 | −0.05 |
| 218132_s_at | NM_024075 | TSEN34 | 79042 | 0.086 | 8.439 | 8.43 | 8.426 | 8.33 | 8.38 | 8.328 | −0.06 |
| 218136_s_at | NM_018579 | SLC25A37 | 51312 | −0.1808 | 5.346 | 5.29 | 5.077 | 5.25 | 5.02 | 4.943 | −0.151 |
| 218138_at | NM_018848 | MKKS | 8195 | −0.0656 | 8.773 | 8.8 | 8.872 | 8.92 | 9.09 | 8.969 | 0.108 |
| 218141_at | NM_022066 | UBE2O | 63893 | −0.0178 | 4.232 | 4.13 | 4.129 | 4.09 | 4.55 | 4.493 | −0.068 |
| 218145_at | NM_021158 | TRIB3 | 57761 | 0.1015 | 10.23 | 10.2 | 10.43 | 10.4 | 11.2 | 11.32 | 0.226 |
| 218148_at | NM_025082 | CENPT | 80152 | −0.0883 | 3.477 | 3.45 | 3.336 | 3.26 | 3.31 | 3.514 | −0.169 |
| 218169_at | NM_018052 | VAC14 | 55697 | 0.065 | 4.833 | 4.42 | 4.468 | 4.67 | 5.02 | 5.246 | −0.059 |
| 218181_s_at | NM_017792 | MAP4K4 | 9448 | 0.0449 | 7.334 | 7.44 | 7.312 | 7.39 | 7.16 | 7.097 | −0.04 |
| 218195_at | NM_024573 | C6orf211 | 79624 | −0.0781 | 7.597 | 7.74 | 7.645 | 7.84 | 7.83 | 7.896 | 0.074 |
| 218197_s_at | NM_018002 | OXR1 | 55074 | 0.0966 | 7.555 | 7.6 | 7.745 | 7.65 | 7.12 | 7.112 | 0.12 |
| 218233_s_at | NM_017601 | PRICKLE4 /// TOMM6 | 100188893 /// 29964 | 0.0608 | 11.21 | 11.3 | 11.17 | 11.2 | 11.4 | 11.34 | −0.052 |
| 218235_s_at | NM_016037 | UTP11L | 51118 | 0.0894 | 8.453 | 8.52 | 8.577 | 8.61 | 8.73 | 8.777 | 0.106 |
| 218246_at | NM_024544 | MUL1 | 79594 | 0.0245 | 5.438 | 5.41 | 5.403 | 5.28 | 5.29 | 5.085 | −0.085 |
| 218265_at | NM_024077 | SEC1SBP2 | 79048 | 0.1158 | 5.014 | 5.27 | 4.972 | 5.12 | 5.45 | 5.3 | −0.096 |
| 218270_at | NM_024540 | MRPL24 | 79590 | −0.0578 | 7.097 | 7.21 | 7.07 | 7.21 | 7.13 | 7.198 | −0.016 |
| 218292_s_at | NM_016203 | PRKAG2 | 51422 | 0.3777 | 5.274 | 5.28 | 5.346 | 5.29 | 6.49 | 6.294 | 0.041 |
| 218321_x_at | NM_016086 | STYXL1 | 51657 | 0.1089 | 7.022 | 7.2 | 6.996 | 7.01 | 7.36 | 7.28 | −0.108 |
| 218328_at | NM_016035 | COQ4 | 51117 | 0.1096 | 6.001 | 6.08 | 6.213 | 6.1 | 6.2 | 6.093 | 0.116 |
| 218343_s_at | NM_012086 | GTF3C3 | 9330 | 0.0288 | 7.793 | 7.88 | 7.857 | 7.84 | 7.61 | 7.632 | 0.011 |
| 218347_at | NM_018264 | TYW1 | 55253 | 0.1284 | 7.172 | 7.05 | 7.224 | 7.11 | 7.08 | 6.971 | 0.054 |
| 218364_at | NM_017724 | LRRFIP2 | 9209 | 0.0922 | 6.819 | 6.95 | 6.838 | 6.58 | 7.69 | 7.932 | −0.177 |
| 218402_s_at | NM_022081 | HPS4 | 89781 | −0.3552 | 3.964 | 4.09 | 3.945 | 4.09 | 3.96 | 3.929 | −0.006 |
| 218427_at | NM_006643 | SDCCAG3 | 10807 | 0.0188 | 6.76 | 6.71 | 6.843 | 6.72 | 7.54 | 7.593 | 0.044 |
| 218431_at | NM_022067 | C14orf133 | 63894 | −0.0062 | 6.5 | 6.61 | 6.673 | 6.45 | 6.32 | 6.102 | 0.008 |
| 218480_at | NM_021831 | AGBL5 | 60509 | −0.1856 | 6.585 | 6.34 | 6.599 | 6.36 | 6.27 | 5.812 | 0.016 |
| 218482_at | NM_020189 | ENY2 | 56943 | 0.0614 | 9.438 | 9.5 | 9.501 | 9.49 | 9.92 | 9.88 | 0.028 |
| 218500_at | NM_016647 | C8orf55 | 51337 | 0.1876 | 5.065 | 4.83 | 4.813 | 4.72 | 4.69 | 4.617 | −0.18 |
| 218543_s_at | NM_022750 | PARP12 | 64761 | 0.1903 | 5.7 | 5.6 | 5.534 | 5.5 | 5.96 | 6.015 | −0.134 |
| 218555_at | NM_013366 | ANAPC2 | 29882 | −0.1164 | 4.62 | 4.86 | 4.667 | 4.65 | 4.65 | 4.254 | −0.079 |
| 218561_s_at | NM_020408 | LYRM4 | 57128 | 0.1813 | 8.432 | 8.39 | 8.394 | 8.39 | 8.37 | 8.329 | −0.016 |
| 218566_s_at | NM_012124 | CHORDC1 | 26973 | 0.0504 | 6.784 | 6.65 | 6.658 | 6.63 | 7.33 | 7.121 | −0.075 |
| 218578_at | NM_024529 | CDC73 | 79577 | −0.119 | 6.818 | 6.86 | 6.825 | 6.91 | 7.09 | 7.154 | 0.03 |
| 218584_at | NM_024549 | TCTN1 | 79600 | −0.0716 | 5.486 | 5.5 | 5.387 | 5.5 | 4.91 | 4.811 | −0.052 |
| 218596_at | NM_018201 | TBC1D13 | 54662 | 0.0704 | 3.772 | 3.61 | 3.777 | 3.92 | 3.56 | 3.729 | 0.16 |
| 218677_at | NM_020672 | S100A14 | 57402 | −0.0994 | 10.84 | 11 | 10.78 | 11.1 | 10.6 | 10.62 | 0.002 |
| 218678_at | NM_024609 | NES | 10763 | −0.2767 | 2.939 | 3.11 | 3.028 | 3.01 | 2.88 | 2.828 | −0.006 |
| 218680_x_at | NM_016400 | HYPK | 25764 | 0.0029 | 8.772 | 8.69 | 8.756 | 8.73 | 9.01 | 8.976 | 0.013 |
| 218763_at | NM_016930 | STX18 | 53407 | 0.0875 | 7.545 | 7.5 | 7.621 | 7.61 | 7.68 | 7.898 | 0.093 |
| 218767_at | NM_020385 | REXO4 | 57109 | 0.0879 | 5.958 | 6.09 | 6.006 | 6.13 | 6.22 | 6.345 | 0.042 |
| 218810_at | NM_025079 | ZC3H12A | 80149 | −0.1218 | 6.204 | 6.21 | 6.238 | 6.05 | 6.25 | 6.335 | −0.059 |
| 218818_at | NM_004468 | PHL3 | 2275 | −0.2527 | 3.634 | 3.51 | 3.57 | 3.57 | 3.55 | 3.455 | −0.002 |
| 218830_at | NM_016093 | RPL26L1 | 51121 | 0.0109 | 9.427 | 9.35 | 9.444 | 9.39 | 9.84 | 9.842 | 0.024 |
| 218846_at | NM_004830 | MED23 | 9439 | 0.2035 | 7.661 | 7.87 | 7.73 | 7.79 | 7.81 | 7.753 | −0.003 |
| 218847_at | NM_006548 | IGF2BP2 | 10644 | 0.1567 | 9.09 | 9.05 | 8.927 | 8.94 | 9.56 | 9.484 | −0.136 |
| 218850_s_at | NM_014240 | LIMD1 | 8994 | 0.2493 | 3.227 | 3.25 | 3.128 | 3.04 | 3.41 | 3.539 | −0.159 |
| 218914_at | NM_015997 | C1orf66 | 51093 | 0.2612 | 5.51 | 5.73 | 5.714 | 5.7 | 5.69 | 5.585 | 0.088 |
| 218954_s_at | AF298153 | BRF2 | 55290 | −0.2911 | 4.422 | 4.47 | 4.336 | 4.42 | 4.13 | 4.349 | −0.07 |
| 218955_at | NM_018310 | BRF2 | 55290 | 0.063 | 5.455 | 5.25 | 5.243 | 5.32 | 5.2 | 5.188 | −0.07 |
| 218965_s_at | NM_022830 | TUT1 | 64852 | −0.2235 | 3.408 | 3.44 | 3.46 | 3.37 | 3.53 | 3.186 | −0.008 |
| 218966_at | NM_018728 | MYO5C | 55930 | −0.1108 | 8.559 | 8.38 | 8.438 | 8.42 | 8.42 | 8.385 | −0.039 |
| 218978_s_at | NM_018586 | SLC25A37 | 51312 | −0.3084 | 4.128 | 4.33 | 4.392 | 3.9 | 3.66 | 3.96 | −0.079 |
| 218991_at | NM_022070 | HEATR6 | 63897 | −0.0202 | 6.977 | 7.07 | 6.825 | 6.93 | 6.66 | 6.831 | −0.148 |
| 219038_at | NM_024657 | MORC4 | 79710 | −0.1236 | 6.256 | 6.17 | 6.214 | 6.3 | 6.41 | 6.286 | 0.041 |
| 219050_s_at | NM_014205 | ZNHIT2 | 741 | 0.0661 | 3.408 | 3.55 | 3.448 | 2.83 | 3.82 | 3.755 | −0.339 |
| 219062_s_at | NM_017742 | ZCCHC2 | 54877 | 0.1625 | 6.442 | 6.58 | 6.466 | 6.49 | 6.34 | 6.387 | −0.034 |
| 219076_s_at | NM_018663 | PXMP2 | 5827 | 0.0193 | 7.354 | 7.36 | 7.261 | 7.16 | 7.12 | 7.244 | −0.144 |
| 219107_at | NM_021948 | BCAN | 63827 | −0.2721 | 3.501 | 3.64 | 3.284 | 3.27 | 3.52 | 3.419 | −0.293 |
| 219128_at | NM_017880 | C2orf42 | 54980 | 0.1033 | 5.614 | 5.48 | 5.801 | 5.82 | 6.19 | 5.919 | 0.267 |
| 219156_at | NM_018373 | SYNJ2BP | 55333 | −0.0297 | 6.412 | 6.69 | 6.61 | 6.57 | 6.13 | 6.151 | 0.043 |
| 219172_at | NM_024954 | UBTD1 | 80019 | 0.0079 | 3.577 | 3.42 | 3.649 | 3.39 | 3.5 | 3.353 | 0.018 |
| 219175_s_at | NM_017836 | SLC41A3 | 54946 | −0.1391 | 5.705 | 6 | 5.883 | 5.95 | 5.73 | 5.682 | 0.061 |
| 219193_at | NM_018034 | WDR70 | 55100 | 0.1098 | 6.879 | 6.79 | 6.899 | 7 | 6.9 | 6.862 | 0.114 |
| 219215_s_at | NM_017767 | SLC39A4 | 55630 | 0.4813 | 5.27 | 5.33 | 5.646 | 5.42 | 6.18 | 6.19 | 0.232 |
| 219217_at | NM_024678 | NARS2 | 79731 | 0.0806 | 6.815 | 6.73 | 7.045 | 6.89 | 7.32 | 7.121 | 0.193 |
| 219221_at | NM_024724 | ZBTB38 | 253461 | 0.1205 | 7.786 | 7.74 | 7.655 | 7.78 | 8.11 | 8.017 | −0.044 |
| 219227_at | NM_024565 | CCNJL | 79616 | −0.1392 | 3.527 | 3.75 | 4.007 | 3.77 | 3.6 | 3.687 | 0.25 |
| 219354_at | NM_018316 | KLHL26 | 55295 | −0.1477 | 4.293 | 4 | 4.022 | 4.14 | 4.01 | 4.108 | −0.067 |
| 219357_at | NM_014027 | GTPBP1 | 9567 | 0.178 | 6.271 | 6.1 | 6.265 | 6.3 | 6.44 | 6.51 | 0.095 |
| 219435_at | NM_025099 | C17orf68 | 80169 | 0.3058 | 4.398 | 4.4 | 4.53 | 4.43 | 4.36 | 4.322 | 0.083 |
| 219456_s_at | AW027923 | RIN3 | 79890 | −0.0875 | 2.979 | 2.95 | 3.137 | 2.95 | 2.99 | 3.011 | 0.078 |
| 219457_s_at | NM_024832 | RIN3 | 79890 | 0.1198 | 3.235 | 3.34 | 3.426 | 3.42 | 3.57 | 3.394 | 0.135 |
| 219459_at | NM_018082 | POLR3B | 55703 | 0.4337 | 6.908 | 6.84 | 6.889 | 6.97 | 7.31 | 7.253 | 0.055 |
| 219468_s_at | NM_017949 | CUEDC1 | 404093 | 0.2959 | 3.731 | 3.74 | 3.779 | 3.67 | 3.97 | 3.877 | −0.008 |
| 219475_at | NM_013370 | OSGIN1 | 29948 | −0.2135 | 3.168 | 3.33 | 3.114 | 3.14 | 3.34 | 3.11 | −0.12 |
| 219489_s_at | NM_017821 | NXN | 64359 | 0.139 | 11.1 | 11 | 11.09 | 11 | 11.1 | 11.1 | −0.004 |
| 219495_s_at | NM_013256 | ZNF180 | 7733 | 0.0803 | 6.007 | 6.04 | 5.968 | 6.17 | 6.29 | 6.108 | 0.048 |
| 219500_at | NM_013246 | CLCF1 | 23529 | 0.0963 | 3.826 | 3.7 | 4.039 | 3.93 | 3.97 | 4.139 | 0.222 |
| 219513_s_at | NM_005490 | SH2D3A | 10045 | 0.0864 | 4.975 | 4.59 | 4.779 | 4.74 | 5.42 | 5.167 | −0.026 |
| 219543_at | NM_022129 | PBLD | 64081 | 0.2541 | 4.297 | 4.14 | 4.345 | 4.11 | 4.21 | 3.947 | 0.007 |
| 219572_at | NM_037954 | CADPS2 | 93664 | −0.1095 | 3.559 | 3.23 | 3.243 | 3.4 | 3.42 | 3.218 | −0.069 |
| 219577_s_at | NM_019112 | ABCA7 | 10347 | 0.062 | 3.487 | 3.53 | 3.536 | 3.38 | 3.27 | 3.608 | −0.05 |
| 219610_at | NM_022448 | RGNEF | 64283 | 0.1517 | 6.897 | 7 | 6.916 | 6.7 | 6.39 | 6.426 | −0.145 |
| 219631_at | NM_024937 | LRP12 | 29967 | 0.0264 | 5.84 | 5.83 | 5.723 | 5.83 | 5.73 | 5.624 | −0.059 |
| 219677_st | NM_025106 | SPSB1 | 80176 | 0.1035 | 3.965 | 3.9 | 4.059 | 3.83 | 4.04 | 4.064 | 0.012 |
| 219692_at | NM_024507 | KREMEN2 | 79412 | −0.1984 | 3.658 | 3.88 | 3.914 | 3.9 | 3.65 | 3.694 | 0.135 |
| 219710_at | NM_024577 | SH3TC2 | 79628 | 0.2908 | 2.9 | 2.96 | 3.28 | 3.02 | 3.78 | 3.526 | 0.217 |
| 239742_at | NM_030567 | PRR7 | 80758 | 0.1231 | 3.954 | 3.34 | 3.385 | 3.44 | 3.95 | 4.11 | −0.238 |
| 219758_at | NM_024926 | TTC26 | 79989 | −0.4278 | 3.91 | 4.1 | 4.205 | 4.04 | 3.64 | 3.322 | 0.117 |
| 219783_at | NM_017877 | C2orf18 | 54978 | −0.1331 | 5.05 | 4.82 | 4.779 | 5.01 | 4.61 | 4.815 | −0.041 |
| 219784_at | NM_024735 | FBXO31 | 79791 | 0.1493 | 4.413 | 4.45 | 4.252 | 4.39 | 4.79 | 4.733 | −0.108 |
| 219785_s_at | NM_024735 | FBXO31 | 79791 | 0.5051 | 3.878 | 4.02 | 3.956 | 3.99 | 4.55 | 4.055 | 0.028 |
| 219794_at | NM_018289 | VPS53 | 55275 | −0.0039 | 3.27 | 3.09 | 2.991 | 3.28 | 2.95 | 3.165 | −0.047 |
| 219801_at | NM_030580 | ZNF34 | 80778 | 0.3078 | 3.367 | 3.36 | 3.534 | 3.27 | 3.61 | 3.596 | 0.04 |
| 219816_s_at | NM_018107 | RBM23 | 55147 | −0.087 | 7.065 | 7.14 | 6.968 | 7.05 | 6.26 | 6.267 | −0.095 |
| 219830_at | NM_030665 | RAI1 | 10743 | 0.0994 | 2.912 | 2.91 | 3.03 | 3.1 | 3.08 | 3.241 | 0.156 |
| 239831_at | NM_016508 | CDKL3 | 51265 | 0.2585 | 5.565 | 5.57 | 5.475 | 5.6 | 5.25 | 5.114 | −0.032 |
| 219842_at | NM_019087 | ARL15 | 54622 | −0.0404 | 3.097 | 3.01 | 3.176 | 3.17 | 2.97 | 3.333 | 0.12 |
| 219862_s_at | NM_012336 | NARF | 26502 | −0.0315 | 7.2 | 7.08 | 7.222 | 7.18 | 6.89 | 7.122 | 0.061 |
| 219899_x_at | NM_014434 | NDOR1 | 27158 | 0.0229 | 3.53 | 3.02 | 3.433 | 3.37 | 3.61 | 3.563 | 0.124 |
| 219901_at | NM_018351 | FGD6 | 55785 | 0.0743 | 6.584 | 6.81 | 6.778 | 6.78 | 6.39 | 6.748 | 0.081 |
| 219907_at | NM_005653 | FRS3 | 10817 | −0.0743 | 2.969 | 3.09 | 2.916 | 3.05 | 2.95 | 2.88 | −0.045 |
| 219940_s_at | NM_018386 | PCID2 | 55795 | 0.0963 | 6.927 | 6.99 | 7.033 | 6.83 | 6.86 | 6.791 | −0.026 |
| 219944_at | NM_024692 | CLIP4 | 79745 | 0.1918 | 4.889 | 5.14 | 5.227 | 5.53 | 5.89 | 5.251 | 0.364 |
| 220002_at | NM_018012 | KIF26B | 55083 | −0.0922 | 3.014 | 3.06 | 3.194 | 3.04 | 3.22 | 3.141 | 0.08 |
| 220007_at | NM_024770 | METTL8 | 79828 | 0.2206 | 6.513 | 6.62 | 6.656 | 6.49 | 7.06 | 6.682 | 0.003 |
| 220020_at | NM_022098 | XPNPEP3 | 63929 | 0.0122 | 4.777 | 4.4 | 4.846 | 4.85 | 4.8 | 4.632 | 0.258 |
| 220024_s_at | NM_020956 | PRX | 57716 | −0.0521 | 3.167 | 3.2 | 3.409 | 3.35 | 3.34 | 3.296 | 0.198 |
| 220043_s_at | NM_005929 | MFI2 | 4241 | 0.1415 | 2.922 | 2.94 | 2.719 | 2.92 | 3.04 | 3.14 | −0.112 |
| 220046_s_at | NM_020307 | CCNL1 | 57018 | 0.0105 | 7.767 | 7.85 | 7.748 | 7.77 | 7.9 | 7.961 | −0.048 |
| 220103_s_at | NM_016067 | MRPS18C | 51023 | 0.1261 | 3.645 | 3.45 | 3.515 | 3.33 | 3.36 | 3.305 | −0.124 |
| 220114_s_at | NM_017564 | STAB2 | 55576 | −0.1253 | 3.373 | 3.25 | 2.987 | 2.99 | 3.37 | 3.12 | −0.324 |
| 220166_at | NM_020348 | CNNM1 | 26507 | −0.1239 | 2.998 | 3.04 | 3.01 | 3.03 | 3.1 | 3.122 | 0.002 |
| 220172_at | NM_025000 | C2orf37 | 80067 | −0.1552 | 3.116 | 3.15 | 3.014 | 3.04 | 3.36 | 3.184 | −0.104 |
| 220208_at | NM_017587 | ADAWTS13 | 11093 | −0.1104 | 3.514 | 3.51 | 3.885 | 3.49 | 3.48 | 3.387 | 0.175 |
| 220227_at | NM_024883 | CDH4 | 1002 | 0.0558 | 3.346 | 3.36 | 3.431 | 3.57 | 4.57 | 4.743 | 0.148 |
| 220228_at | AB030653 | AP4E1 | 23431 | 0.175 | 2.601 | 2.76 | 2.752 | 2.83 | 2.82 | 2.596 | 0.111 |
| 220229_s_at | NM_007347 | AP4E1 | 23431 | 0.1251 | 3.049 | 3.47 | 3.296 | 3.3 | 3.37 | 3.184 | 0.039 |
| 220248_x_at | NM_018839 | NSFL1C | 55968 | −0.0368 | 8.608 | 8.61 | 8.553 | 8.55 | 8.62 | 8.722 | −0.056 |
| 220253_s_at | NM_013437 | LRP12 | 29967 | −0.1193 | 5.937 | 5.87 | 6.04 | 5.83 | 5.98 | 5.559 | 0.03 |
| 220254_at | NM_013437 | LRP12 | 29967 | 0.0197 | 5.621 | 5.46 | 5.63 | 5.39 | 5.61 | 5.451 | −0.033 |
| 220271_x_at | NM_022785 | EFCAB6 | 64800 | −0.0822 | 3.021 | 3.09 | 3.207 | 3.07 | 3.02 | 3.048 | 0.085 |
| 220312_at | NM_017708 | FAM83E | 54854 | 0.0708 | 2.693 | 2.72 | 2.823 | 2.79 | 2.75 | 2.843 | 0.1 |
| 220329_s_at | NM_017909 | RMND1 | 55005 | −0.0654 | 6.846 | 6.95 | 6.977 | 6.75 | 7.04 | 7.044 | −0.037 |
| 220349_s_at | NM_022759 | FLJ21865 | 64772 | −0.2058 | 5.373 | 5.26 | 5.214 | 5.45 | 4.99 | 5.167 | −0.017 |
| 220395_at | NM_018602 | DNAJA4 | 55466 | −0.195 | 3.974 | 4.16 | 3.848 | 4.09 | 3.74 | 4.038 | −0.095 |
| 220434_at | NM_024876 | ADCK4 | 79934 | 0.1124 | 3.072 | 2.88 | 2.991 | 3.12 | 2.79 | 3.024 | 0.082 |
| 220439_at | NM_024892 | RIN3 | 79890 | −0.1465 | 3.155 | 3.05 | 3.111 | 3.03 | 3.02 | 3.08 | −0.031 |
| 220546_at | NM_024891 | MLL | 4297 | 0.0312 | 3.055 | 3.02 | 3.098 | 3.05 | 3.02 | 3.33 | 0.032 |
| 220588_at | NM_017843 | BCAS4 | 55653 | −0.0862 | 5.459 | 5.4 | 5.566 | 5.47 | 5.49 | 5.449 | 0.086 |
| 220610_s_at | NM_006309 | LRRFIP2 | 9209 | −0.0016 | 6.946 | 7.05 | 6.778 | 6.97 | 7.49 | 7.515 | −0.124 |
| 220688_s_at | NM_016183 | MRTO4 | 51154 | −0.1678 | 7.888 | 7.93 | 7.977 | 7.89 | 8.14 | 8.152 | 0.027 |
| 220731_s_at | NM_018090 | NECAP2 | 55707 | 0.025 | 5.813 | 5.82 | 5.689 | 5.85 | 5.6 | 5.614 | −0.046 |
| 220744_s_at | NM_018262 | IFT122 | 55764 | 0.2769 | 4.46 | 4.55 | 4.573 | 4.93 | 4.86 | 4.718 | 0.245 |
| 220801_s_at | NM_016527 | HAO2 | 51179 | −0.1075 | 3.056 | 2.98 | 2.823 | 2.82 | 2.73 | 2.791 | −0.2 |
| 220947_s_at | NM_015527 | TBC1D10B | 26000 | −0.0807 | 4.342 | 4.55 | 4.412 | 4.24 | 4.28 | 4.301 | −0.119 |
| 220973_s_at | NM_030974 | SHARPIN | 81858 | −0.124 | 5.909 | 5.69 | 5.684 | 5.83 | 5.9 | 5.914 | −0.043 |
| 220986_s_at | NM_030953 | TIGD6 | 81789 | 0.0252 | 3.324 | 3.32 | 3.015 | 3.13 | 3.06 | 3.103 | −0.25 |
| 221037_s_at | NM_031291 | SLC2SA31 | 83447 | −0.0928 | 2.56 | 2.34 | 2.597 | 2.6 | 2.48 | 2.625 | 0.149 |
| 221049_s_at | NM_013274 | POLL | 27343 | 0.1167 | 5.437 | 5.13 | 5.372 | 4.94 | 5.12 | 4.934 | −0.13 |
| 221206_at | NM_024521 | PMS2 /// PMS2CL | 441194 /// 5395 | 0.1886 | 5.1 | 5.13 | 4.95 | 5.04 | 5.3 | 5.064 | −0.123 |
| 221211_s_at | NM_020152 | C21orf7 | 56911 | 0.0167 | 3.138 | 3.08 | 3.141 | 3.33 | 3.02 | 3.279 | 0.13 |
| 221290_s_at | NM_016473 | MUM1 | 84939 | 0.1791 | 3.974 | 4.13 | 3.748 | 3.83 | 4.71 | 4.257 | −0.263 |
| 221307_at | NM_014592 | KCNIP1 | 30820 | −0.1317 | 3.228 | 3.18 | 3.282 | 3.12 | 3.42 | 3.314 | −0.002 |
| 221335_x_at | NM_019108 | C19orf61 | 56006 | 0.1698 | 4.806 | 5.03 | 4.984 | 4.61 | 4.58 | 4.758 | −0.123 |
| 221438_s_at | NM_031275 | TEX12 | 56158 | −0.0839 | 2.775 | 2.86 | 2.893 | 2.79 | 2.71 | 2.796 | 0.024 |
| 221455_s_at | NM_030753 | WNT3 | 7473 | −0.1502 | 2.914 | 2.85 | 2.833 | 2.96 | 3.08 | 2.99 | 0.016 |
| 221499_s_at | AK_026970 | STX16 | 8675 | 0.0224 | 7.199 | 7.32 | 7.288 | 7.32 | 7.18 | 7.1 | 0.048 |
| 221500_s_at | BE782754 | STX16 | 8675 | 0.0026 | 9.153 | 8.99 | 90.84 | 9.11 | 9.17 | 9.188 | 0.028 |
| 221534_at | AF073483 | C11orf68 | 83638 | 0.1953 | 4.432 | 4.66 | 4.481 | 4.48 | 4.63 | 4.661 | −0.066 |
| 221571_at | AI721219 | TRAF3 | 7187 | 0.1628 | 6.359 | 6.23 | 6.198 | 6.25 | 6.6 | 6.711 | −0.067 |
| 221614_s_at | BC005153 | RPH3AL | 9501 | −0.0583 | 3.327 | 2.99 | 3.306 | 3.08 | 2.83 | 3.312 | 0.036 |
| 221619_s_at | AF189289 | MTCH1 | 23787 | 0.0728 | 11.07 | 11 | 10.93 | 10.9 | 10.9 | 10.89 | −0.114 |
| 221623_at | AF229053 | BCAN | 63827 | −0.1908 | 2.641 | 2.86 | 3.062 | 2.87 | 2.65 | 2.95 | 0.216 |
| 221638_s_at | AF008937 | STX16 | 8675 | 0.0029 | 4.88 | 4.98 | 4.735 | 4.3 | 4.77 | 4.599 | −0.409 |
| 221676_s_at | BC002342 | CORO1C | 23603 | 0.3129 | 8.771 | 8.69 | 8.82 | 8.62 | 9.48 | 9.48 | −0.008 |
| 221702_s_at | AF353992 | TM2D3 | 80213 | −0.0469 | 8.263 | 8.3 | 8.209 | 8.37 | 7.92 | 8.107 | 0.006 |
| 221707_s_at | BC006116 | VPS53 | 55275 | 0.1568 | 2.972 | 2.98 | 3.259 | 2.96 | 3.1 | 3.287 | 0.132 |
| 221809_at | AB040897 | RANBP10 | 57610 | −0.0872 | 3.698 | 3.56 | 3.779 | 3.83 | 3.58 | 3.687 | 0.174 |
| 221814_at | BF511315 | GPR124 | 25960 | −0.2089 | 3.233 | 3.53 | 3.314 | 3.24 | 3.76 | 3.412 | −0.104 |
| 221845_s_at | AI655698 | CLPB | 81570 | −0.1025 | 4.712 | 5.27 | 4.871 | 5.32 | 4.99 | 4.827 | 0.105 |
| 221854_at | AI378979 | PKP1 | 5317 | −0.2137 | 7.993 | 7.97 | 8.003 | 8.13 | 8.49 | 8.588 | 0.084 |
| 221865_at | BF969986 | C9orf91 | 203197 | 0.3971 | 5.287 | 5.09 | 5.133 | 5.3 | 5.6 | 5.609 | 0.028 |
| 221870_at | AI417917 | EHD2 | 30846 | 0.2725 | 6.838 | 6.96 | 6.938 | 7.1 | 7.18 | 7.23 | 0.119 |
| 221881_s_at | AI638420 | CLIC4 | 25932 | −0.0989 | 6.248 | 6.35 | 6.218 | 6.35 | 6.65 | 6.793 | −0.017 |
| 221891_x_at | AA704004 | HSPA8 | 3312 | −0.4272 | 11.84 | 11.8 | 11.87 | 11.8 | 11.5 | 11.54 | −3E−05 |
| 221897_at | AA205660 | TRIM52 | 84851 | −0.0278 | 4.365 | 4.46 | 4.49 | 4.38 | 4.09 | 4.073 | 0.023 |
| 221899_at | AI809961 | N4BP2L2 | 10443 | −0.1238 | 8.365 | 8.23 | 8.359 | 8.22 | 8.11 | 8.053 | −0.009 |
| 221920_s_at | BE677761 | SLC25A37 | 51312 | −0.2357 | 5.139 | 4.86 | 5.204 | 4.93 | 4.59 | 4.72 | 0.07 |
| 221926_s_at | BF196320 | IL17RC | 84818 | −0.0717 | 3.411 | 3.49 | 3.531 | 3.52 | 3.8 | 3.469 | 0.075 |
| 221960_s_at | AI89609 | RAB2A | 5862 | 0.1991 | 5.876 | 5.63 | 5.608 | 5.99 | 5.9 | 5.087 | 0.045 |
| 221990_at | AI948472 | PAX8 | 7849 | −0.0254 | 2.554 | 2.67 | 2.842 | 2.8 | 2.66 | 2.528 | 0.21 |
| 221998_s_at | BF062886 | VRK3 | 51231 | 0.1508 | 6.185 | 6.49 | 6.299 | 6.59 | 6.48 | 6.611 | 0.107 |
| 221999_at | BF062886 | VRK3 | 51231 | 0.2228 | 3.367 | 3.33 | 3.204 | 3.56 | 3.37 | 3.622 | 0.036 |
| 222010_at | BF224073 | TCP1 | 6950 | 0.0457 | 7.35 | 7.17 | 7.176 | 7.04 | 7.33 | 7.411 | −0.152 |
| 222011_s_at | BF224073 | TCP1 | 6950 | −0.0321 | 6.954 | 6.91 | 6.92 | 6.94 | 6.82 | 7.027 | −0.001 |
| 222035_s_at | AI984479 | PAPOLA | 10914 | 0.0693 | 8.815 | 8.87 | 8.756 | 8.83 | 8.89 | 8.908 | −0.049 |
| 222043_at | AI982754 | CLU | 1191 | −0.0133 | 2.794 | 2.73 | 2.85 | 2.77 | 3 | 2.886 | 0.051 |
| 222154_s_at | AK002064 | LOC26010 | 26010 | 0.2266 | 7.844 | 7.83 | 7.712 | 7.89 | 7.9 | 7.949 | −0.033 |
| 222169_x_at | N71739 | SH2D3A | 10045 | 0.0425 | 4.514 | 4.55 | 4.701 | 4.61 | 4.58 | 4.165 | 0.127 |
| 222176_at | AK021487 | PTEN | 5728 | −0.0787 | 2.91 | 3.17 | 3.246 | 3.19 | 3.04 | 2.957 | 0.178 |
| 222188_at | AK023069 | C9orf156 | 51531 | −0.1117 | 2.85 | 2.83 | 2.773 | 2.89 | 2.63 | 2.911 | −0.012 |
| 222195_s_at | AK023069 | C9orf156 | 51531 | 0.0698 | 5.906 | 6.02 | 6.143 | 5.83 | 6 | 5.876 | 0.023 |
| 222220_s_at | AK027245 | TSNAXIP1 | 55815 | 0.047 | 3.4 | 3.35 | 3.322 | 3.31 | 3.57 | 3.611 | −0.058 |
| 222231_s_at | AK025328 | LRRCS9 | 55379 | −0.2769 | 9.158 | 9.09 | 9.226 | 9.17 | 9.18 | 9.231 | 0.073 |
| 222255_at | AB046840 | PRX | 57716 | 0.0574 | 2.483 | 2.6 | 2.442 | 2.43 | 2.56 | 2.651 | −0.107 |
| 222305_at | AW975638 | HK2 | 3099 | 0.1662 | 5.452 | 5.27 | 5.498 | 5.29 | 5.27 | 5.397 | 0.032 |
| 222346_at | AI633741 | LAMA1 | 284217 | −0.0677 | 4.089 | 3.68 | 3.785 | 3.88 | 3.8 | 3.75 | −0.051 |
| 222348_at | AW971134 | MAST4 | 375449 | −0.0669 | 5.36 | 5 | 4.938 | 5.05 | 5.19 | 5.26 | −0.187 |
| 222353_at | AV720842 | LIMD1 | 8994 | 0.1385 | 3.137 | 2.9 | 3.147 | 2.97 | 3.09 | 3.233 | 0.043 |
| 222383_s_at | AW003512 | ALOXE3 | 59344 | 0.5771 | 2.947 | 2.9 | 2.889 | 2.92 | 3.16 | 3.082 | −0.019 |
| 31846_at | AW003733 | RHOD | 29984 | 0.1137 | 9.075 | 8.98 | 9.069 | 8.89 | 9.44 | 9.384 | −0.048 |
| 31861_at | L14754 | IGHMBP2 | 3508 | −0.1564 | 5.449 | 5.74 | 5.247 | 5.51 | 5.17 | 5.239 | −0.219 |
| 32094_at | AB017915 | CHST3 | 9469 | 0.1262 | 4.478 | 4.46 | 4.223 | 4.57 | 4.76 | 4.65 | −0.077 |
| 33132_at | U37012 | CPSF1 | 29894 | 0.0612 | 5.969 | 5.92 | 6.028 | 5.95 | 5.86 | 6.173 | 0.046 |
| 34478_at | X79780 | RAB11B | 9230 | 0.1713 | 3.081 | 3.1 | 3.086 | 3.07 | 3.25 | 2.994 | −0.015 |
| 36865_at | AB018302 | ANGEL1 | 23357 | 0.0717 | 4.183 | 4.11 | 4.328 | 4.28 | 4.47 | 4.292 | 0.154 |
| 37005_at | D28124 | NBL1 | 4681 | 0.0765 | 6.247 | 6.4 | 6.426 | 6.48 | 6.35 | 6.242 | 0.13 |
| 37566_at | AB028968 | KIAA1045 | 23349 | 0.0109 | 2.822 | 2.8 | 2.766 | 2.67 | 2.66 | 2.688 | −0.094 |
| 37860_at | AL049942 | ZNF337 | 26152 | −0.0512 | 6.692 | 6.71 | 6.588 | 6.6 | 6.56 | 6.588 | −0.108 |
| 37872_at | AF072468 | JRK | 8629 | 0.0734 | 4.566 | 4.73 | 4.639 | 4.52 | 4.44 | 4.276 | −0.071 |
| 38269_at | AL050147 | PRKD2 | 25865 | 0.0984 | 6.771 | 6.87 | 6.842 | 6.84 | 6.71 | 6.796 | 0.019 |
| 38447_at | U08438 | ADRBK1 | 156 | 0.048 | 4.45 | 4.55 | 4.247 | 4.59 | 4.33 | 4.31 | −0.078 |
| 38918_at | AF083105 | SOX13 | 9580 | 0.1083 | 3.661 | 3.57 | 3.66 | 3.95 | 3.72 | 3.714 | 0.19 |
| 39817_s_at | AF040105 | C6orf108 | 10591 | 0.169 | 7.312 | 7.45 | 7.362 | 7.38 | 7.42 | 7.421 | −0.008 |
| 40148_at | U62325 | APBB2 | 323 | 0.0729 | 4.16 | 4.07 | 4.134 | 4.17 | 5.24 | 5.224 | 0.033 |
| 40273_at | AA485440 | SPHK2 | 56848 | 0.2275 | 4.169 | 4.28 | 4.348 | 4.17 | 4.41 | 4.476 | 0.038 |
| 41220_at | AB023208 | 10-Sep | 10801 | 0.0528 | 10.6 | 10.7 | 10.64 | 10.6 | 10.4 | 10.33 | −0.005 |
| 41657_at | AF035625 | STK11 | 6794 | 0.0905 | 4.037 | 4.09 | 4.291 | 4.04 | 4.1 | 4.173 | 0.097 |
| 41660_at | AL031588 | CELSR1 | 9620 | −0.0573 | 5.835 | 5.83 | 5.941 | 5.75 | 5.96 | 5.784 | 0.015 |
| 44696_at | AA915989 | TBC1D13 | 54662 | 0.0008 | 5.287 | 5.31 | 5.313 | 5.38 | 5.24 | 4.973 | 0.049 |
| 45297_at | AI417917 | EHD2 | 30846 | 0.0366 | 6.381 | 6.31 | 6.224 | 6.43 | 6.64 | 6.524 | −0.018 |
| 47530_at | AA748492 | C9orf156 | 51531 | 0.0371 | 5.564 | 5.52 | 5.675 | 5.57 | 5.67 | 5.577 | 0.077 |
| 53987_at | AL041852 | RANBP10 | 57610 | −0.0449 | 4.272 | 4.18 | 4.115 | 4.2 | 4.17 | 4.104 | −0.065 |
| 54037_at | AL041451 | HPS4 | 89781 | 0.0945 | 4.08 | 4.19 | 4.151 | 4.2 | 4.09 | 3.796 | 0.043 |
| 60471_at | AA625133 | RIN3 | 79890 | 0.202 | 4.399 | 4.07 | 4.297 | 4.21 | 4.54 | 4.671 | 0.015 |
| 64440_at | AI560217 | IL17RC | 84818 | −0.0134 | 4.57 | 4.37 | 4.336 | 4.35 | 4.17 | 4.238 | −0.124 |
| 65493_at | AA555088 | HEATR6 | 63897 | −0.0342 | 5.471 | 5.61 | 5.568 | 5.62 | 5.63 | 5.423 | 0.054 |
| 65635_at | AL044097 | FLJ21865 | 64772 | 0.0206 | 5.206 | 5.22 | 5.292 | 5.16 | 4.95 | 4.991 | 0.01 |
| 65718_at | AI655903 | GPR124 | 25960 | 0.0934 | 3.16 | 3.22 | 3.237 | 3.18 | 3.31 | 3.405 | 0.023 |
| 91920_at | AI205180 | BCAN | 63827 | −0.1043 | 3.469 | 3.34 | 3.379 | 3.33 | 3.34 | 3.252 | −0.049 |

| | | | | BPLER | | | | | | | MCF7 | |
|---|---|---|---|---|---|---|---|---|---|---|---|---|
| | | | | (hA6 | | | | | | | (GFP | MCF7 |
| | Representative | | | vs | | | | | | | vs | (hA6 vs |
| Probe Set ID | Public ID | Gene Symbol | Entrez Gene | SCR) | SCR_MCF7_A | SCR_MCF7_B | GFP_MCF7_A | GFP_MCF7_B | ha6_MCF7 1 | ha6_MCF72 | SCR) | SCR) |
| 117_at | X51757 | HSPA6 | 3310 | 0.006 | 2.96 | 3.04 | 3.063 | 3.116 | 3.004 | 2.965 | 0.092 | −0.014 |
| 121_at | X69699 | RAX8 | 7849 | 0.074 | 5.46 | 5.536 | 5.43 | 5.313 | 5.237 | 5.39 | −0.125 | −0.183 |
| 1487_at | L38487 | ESRRA | 2101 | 0.117 | 5.05 | 5.081 | 5.335 | 5.364 | 5.307 | 5.47 | 0.282 | 0.321 |
| 200002_at | NM_007209 | RPL35 | 11224 | −0.11 | 11.7 | 11.79 | 11.78 | 11.77 | 11.8 | 11.81 | 0.034 | 0.068 |
| 200017_at | NM_002954 | RPS27A /// UBB /// | 6233 /// 7314 /// | −0.13 | 12.5 | 12.54 | 12.52 | 12.52 | 12.39 | 12.37 | −0.012 | −0.154 |
| | | UBC | 7316 | | | | | | | | | |
| 200019_s_at | NM_001997 | FAU | 2197 | −0.03 | 12.2 | 12.21 | 12.18 | 12.22 | 12.34 | 12.34 | −0.018 | 0.123 |
| 200022_at | NM_000979 | RPL18 | 6141 | −0.18 | 12.4 | 12.48 | 12.41 | 12.45 | 12.35 | 12.41 | −0.024 | −0.072 |
| 200024_at | NM_001009 | RPSS | 6193 | −0.06 | 11.7 | 11.7 | 11.75 | 11.67 | 11.6 | 11.68 | −0.011 | −0.082 |
| 200037_s_at | NM_016587 | CBX3 /// LOC653972 | 11335 /// 653972 | −0.84 | 11.1 | 11.06 | 11.05 | 10.98 | 9.696 | 9.734 | −0.046 | −1.351 |
| 200049_at | NM_007067 | MYST2 | 11143 | −0.07 | 6.96 | 6.874 | 7.185 | 7.133 | 7.017 | 6.907 | 0.239 | 0.043 |
| 200064_at | AF275719 | HSP90AB1 | 3326 | −0.69 | 11.4 | 11.32 | 11.3 | 11.36 | 10.91 | 10.94 | −0.02 | −0.426 |
| 200067_x_at | AL078595 | SNX3 | 8724 | −0.06 | 11.4 | 10.3 | 10.27 | 10.31 | 10.55 | 10.53 | −0.041 | 0.21 |
| 200601_at | U48734 | ACTN4 | 81 | 0.58 | 7.58 | 7.501 | 7.44 | 7.543 | 7.616 | 7.51 | −0.048 | 0.022 |
| 200602_at | NM_000484 | APP | 351 | −0.24 | 8.54 | 8.597 | 8.545 | 8.45 | 8.292 | 8.339 | −0.07 | −0.253 |
| 200618_at | NM_006148 | LASP1 | 3927 | −0.38 | 8.7 | 8.663 | 8.608 | 8.65 | 8.551 | 8.653 | −0.05 | −0.077 |
| 200622_x_at | AV685208 | CALM1 /// C4LM2 /// | 801 /// 805 /// 808 | 0.435 | 8.76 | 8.729 | 8.49 | 8.619 | 8.803 | 8.87 | −0.191 | 0.09 |
| | | CALM3 | | | | | | | | | | |
| 200623_s_at | NM_005184 | CALM1 /// CALM2 /// | 801 /// 805 /// 808 | −0.02 | 5.98 | 6.066 | 6.131 | 6.245 | 6.015 | 6.19 | 0.164 | 0.079 |
| | | CALM3 | | | | | | | | | | |
| 200627_at | BC003005 | PTGES3 | 10728 | −0.09 | 11.6 | 11.49 | 11.5 | 11.47 | 11.22 | 11.28 | −0.059 | −0.298 |
| 200632_s_at | NM_006096 | NDRG1 | 10397 | −0.66 | 6.6 | 6.508 | 6.832 | 6.61 | 6.457 | 6.226 | 0.165 | −0.215 |
| 200633_at | NM_018955 | RPS27A /// UBB /// | 6233 /// 7314 /// | −0.37 | 12.8 | 12.88 | 12.82 | 12.84 | 12.76 | 12.74 | 0.007 | −0.073 |
| | | UBC | 7316 | | | | | | | | | |
| 200653_s_at | M27319 | CALM1 /// CALM2 /// | 801 /// 805 /// 808 | −0.13 | 10.7 | 10.66 | 10.56 | 10.5 | 10.53 | 10.58 | −0.147 | −0.119 |
| | | CALM3 | | | | | | | | | | |
| 200655_s_at | NM_006888 | CALM1 /// CALM2 /// | 801 /// 805 /// 808 | 0.033 | 10.4 | 10.31 | 10.27 | 10.28 | 10.13 | 10.15 | −0.077 | −0.211 |
| | | CALM3 | | | | | | | | | | |
| 200664_s_at | BG537255 | DNAJB1 | 3337 | −0.25 | 8.18 | 8.191 | 8.116 | 8.144 | 7.934 | 8.025 | −0.053 | −0.204 |
| 200666_s_at | NM_006145 | DNAJB1 | 3337 | −0.33 | 8.4 | 8.38 | 8.232 | 8.375 | 8.134 | 8.252 | −0.089 | −0.199 |
| 200667_at | BF448062 | UBE2D3 | 7323 | −0.1 | 8.38 | 8.255 | 8.331 | 8.312 | 8.137 | 8.197 | 0.004 | −0.15 |
| 200668_s_at | BC003395 | UBE2D3 | 7323 | 0.015 | 10.2 | 10.24 | 10.28 | 10.28 | 10.13 | 10.11 | 0.057 | −0.097 |
| 200669_s_at | NM_003340 | UBE2D3 | 7323 | 0.123 | 9.59 | 9.492 | 9.523 | 9.533 | 9.69 | 9.66 | −0.011 | 0.136 |
| 200687_s_at | NM_012426 | SF3B3 | 23450 | −0.16 | 8.47 | 8.459 | 8.362 | 8.405 | 8.245 | 8.383 | −0.083 | −0.152 |
| 200688_at | D13642 | SF3B3 | 23450 | 0.012 | 4.09 | 4.059 | 4.067 | 4.149 | 3.98 | 3.938 | 0.034 | −0.115 |
| 200689_x_at | NM_001404 | EEF1G | 1937 | −0.13 | 12.3 | 12.2 | 12.23 | 12.19 | 12.19 | 12.23 | −0.045 | |
| 200696_s_at | NM_000177 | GSN | 2934 | −0.38 | 8.09 | 7.819 | 7.891 | 7.88 | 8.133 | 8.294 | −0.071 | |
| 200707_at | NM_002743 | PRXCSH | 5589 | −0.17 | 6.17 | 6.383 | 6.21 | 6.374 | 6.683 | 6.567 | 0.014 | |
| 200737_at | NM_000791 | PGK1 | 5230 | −0.37 | 8.68 | 8.691 | 8.79 | 8.592 | 8.526 | 8.366 | 0.006 | |
| 200738_s_at | NM_000291 | PGK1 | 5230 | −0.12 | 11.1 | 11.23 | 11.23 | 11.14 | 11.19 | 11.02 | 0.013 | |
| 200753_x_at | BE866585 | SFRS2 | 6427 | 0.03 | 9.3 | 9.266 | 9.257 | 9.125 | 8.756 | 8.989 | −0.091 | |
| 200754_x_at | NM_003016 | SF952 | 6427 | 0.155 | 10.7 | 10.68 | 10.69 | 10.64 | 10.57 | 10.57 | −0.029 | |
| 200768_s_at | BC001686 | MAT2A | 4144 | −0.01 | 8.61 | 8.489 | 8.526 | 8.587 | 8.311 | 8.266 | 0.009 | |
| 200769_s_at | NM_005911 | MAT2A | 4144 | 0.126 | 7 | 6.856 | 6.912 | 6.874 | 6.739 | 6.629 | −0.034 | |
| 200806_s_at | BE256479 | HSPD1 | 3329 | −0.08 | 12 | 11.98 | 11.92 | 12.02 | 11.84 | 11.77 | −0.009 | |
| 200807_s_at | NM_002156 | HSPD1 | 3329 | 0.056 | 12.3 | 12.22 | 12.25 | 12.26 | 12.2 | 12.07 | 0.007 | |
| 200812_at | NM_006429 | CCT7 | 10574 | 0.168 | 10.1 | 10.05 | 10.06 | 10.06 | 9.881 | 9.913 | −0.031 | |
| 200823_x_at | NM_000992 | LOC100131713 /// | 100131713 /// | −0.21 | 11.7 | 11.68 | 11.6 | 11.64 | 11.62 | 11.5 | −0.051 | |
| | | RPL29 /// RPL29P4 | 387101 /// 6159 | | | | | | | | | |
| 200828_s_at | BE871379 | ZNF207 | 7756 | −0.09 | 9.91 | 9.877 | 9.872 | 9.848 | 9.837 | 9.91 | −0.035 | |
| 200829_x_at | NM_003457 | ZNF207 | 7756 | −0.04 | 9.69 | 9.616 | 9.571 | 9.521 | 9.465 | 9.595 | −0.106 | |
| 200847_s_at | NM_016127 | TMEM66 | 51669 | −0.8 | 10.5 | 10.45 | 10.43 | 10.41 | 10.3 | 10.19 | −0.043 | |
| 200854_at | AB028970 | NCOR1 | 9611 | 0.349 | 6.68 | 6.776 | 6.883 | 6.759 | 7.204 | 7.199 | 0.094 | |
| 200857_s_at | NM_006311 | NCOR1 | 9611 | 0.307 | 6.44 | 6.403 | 6.645 | 6.535 | 6.942 | 6.657 | 0.167 | |
| 200873_s_at | NM_006585 | CCT8 | 10694 | −0.17 | 11 | 11.01 | 10.9 | 10.96 | 10.94 | 10.88 | −0.063 | |
| 200877_at | NM_006430 | CCT4 | 10575 | −0.28 | 11.6 | 11.6 | 11.47 | 11.54 | 11.38 | 11.33 | −0.109 | |
| 200880_at | AL534104 | DNAJA1 | 3301 | −0.35 | 8.55 | 8.296 | 8.28 | 8.294 | 8.445 | 8.61 | −0.138 | |
| 200881_s_at | NM_001539 | DNAJA1 | 3301 | −0.64 | 10.4 | 10.49 | 10.35 | 10.47 | 10.02 | 9.976 | −0.047 | |
| 200892_s_at | BC000451 | SFRS10 | 6434 | 0.184 | 9.5 | 9.584 | 9.463 | 9.558 | 9.078 | 9.15 | −0.032 | |
| 200893_at | NM_004593 | SFRS10 | 6434 | 0.185 | 11.1 | 11.08 | 11.02 | 11.02 | 10.8 | 10.74 | −0.063 | |
| 200894_s_at | AA894574 | FKBP4 | 2288 | −0.07 | 8.68 | 8.714 | 8.499 | 8.616 | 7.845 | 8.2 | −0.14 | |
| 290895_s_at | NM_002014 | FXBP4 | 2288 | 0.209 | 9.14 | 9.107 | 9.079 | 9.034 | 8.609 | 8.653 | −0.066 | −0.492 |
| 200896_x_at | NM_004494 | HDGF | 3068 | −0.01 | 10.5 | 10.38 | 10.53 | 10.37 | 10.22 | 10.17 | 0.023 | −0.236 |
| 200910_at | NM_005998 | CCT3 | 7203 | −0.35 | 9.87 | 9.809 | 9.809 | 9.87 | 9.468 | 9.405 | 3E−04 | −0.403 |
| 200912_s_at | NM_001967 | EIF4A2 | 1974 | −0.29 | 11.4 | 11.38 | 11.27 | 11.26 | 11.33 | 11.28 | −0.111 | −0.07 |
| 200936_at | NM_000973 | RPL8 | 6132 | −0.03 | 12.9 | 12.96 | 12.9 | 12.9 | 12.93 | 13.01 | −0.036 | 0.035 |
| 200965_s_at | NM_006720 | ABLIM1 | 3983 | 0.411 | 5.47 | 5.382 | 5.379 | 5.355 | 5.475 | 5.292 | −0.058 | −0.042 |
| 200983_x_at | BF983379 | CD59 | 966 | −0.3 | 9.34 | 9.426 | 9.335 | 9.297 | 9.258 | 9.303 | −0.067 | −0.103 |
| 200984_s_at | X16447 | CD59 | 966 | −0.43 | 8.11 | 8.24 | 8.232 | 8.139 | 7.812 | 7.889 | 0.011 | −0.324 |
| 200985_s_at | NM_000611 | CD59 | 966 | −0.22 | 8.39 | 8.237 | 8.344 | 8.291 | 8.104 | 8.155 | 0.004 | −0.184 |
| 201023_at | NM_005642 | TAF7 | 6879 | 0.495 | 10.1 | 9.998 | 10.04 | 9.892 | 9.977 | 10.15 | −0.069 | 0.029 |
| 201066_at | NM_001916 | CYC1 | 1537 | 0.538 | 9.32 | 9.168 | 9.186 | 9.278 | 9.434 | 9.437 | −0.012 | 0.192 |
| 201079_at | NM_004710 | SYNGR2 | 9144 | 0.052 | 8.55 | 8.653 | 8.65 | 8.63 | 8.505 | 8.497 | 0.039 | −0.1 |
| 201091_s_at | BE748755 | CBX3 /// LOC653972 | 11335 /// 653972 | −0.34 | 9.87 | 9.902 | 9.895 | 9.837 | 9.438 | 9.231 | −0.022 | −0.554 |
| 201129_at | NM_006276 | SFRS7 | 6432 | 0.332 | 8.46 | 8.403 | 8.366 | 8.453 | 8.416 | 8.412 | −0.023 | −0.019 |
| 201132_at | NM_019597 | HNRNPH2 | 3188 | −0.38 | 8.38 | 8.466 | 8.401 | 8.356 | 8.08 | 8.26 | −0.046 | −0.254 |
| 201140_s_at | NM_004583 | RAB5C | 5878 | −0.54 | 7.63 | 7.566 | 7.478 | 7.585 | 6.953 | 7.295 | −0.067 | −0.475 |
| 201156_s_at | AF141304 | RAB5C | 5878 | −0.48 | 7.49 | 7.71 | 7.434 | 7.537 | 7.196 | 7.384 | −0.116 | −0.312 |
| 201162_at | NM_001553 | IGFBP7 | 3490 | −0.48 | 3.81 | 4.06 | 4.175 | 4.165 | 3.714 | 4.147 | 0.237 | −0.003 |
| 201163_s_at | NM_001553 | IGFBP7 | 3490 | −0.39 | 3.44 | 3.39 | 3.341 | 3.54 | 3.061 | 2.967 | 0.024 | −0.402 |
| 201173_x_at | NM_006600 | NUDC | 10726 | 0.451 | 7.88 | 7.699 | 7.822 | 7.802 | 8.044 | 7.832 | 0.02 | 0.146 |
| 201182_s_at | AI761771 | CHD4 | 1108 | 0.023 | 7.24 | 7.173 | 7.112 | 7.164 | 7.434 | 7.313 | −0.07 | 0.166 |
| 201183_s_at | AI613273 | CHD4 | 1108 | −0.03 | 7.82 | 7.914 | 7.931 | 7.916 | 7.807 | 7.691 | 0.055 | −0.12 |
| 201184_s_at | NM_001273 | CHD4 | 1108 | −0.04 | 7.68 | 7.413 | 7.426 | 7.256 | 7.492 | 7.443 | −0.204 | −0.078 |
| 201194_at | NM_003009 | SEPW1 | 6415 | −0.23 | 9.63 | 9.591 | 9.356 | 9.501 | 9.269 | 9.373 | −0.18 | −0.288 |
| 201218_at | N23018 | CTBP2 | 1488 | −0.46 | 9.77 | 9.709 | 9.577 | 9.562 | 8.972 | 8.954 | −0.172 | −0.779 |
| 201219_at | AW269836 | CTBP2 | 1488 | −0.17 | 6.92 | 6.787 | 6.923 | 6.742 | 6.472 | 6.122 | −0.019 | −0.555 |
| 201220_x_at | NM_001329 | CTBP2 | 1488 | −0.08 | 9.95 | 9.854 | 9.914 | 9.847 | 9.692 | 9.669 | −0.023 | −0.224 |
| 201249_at | AI091047 | SLC2A1 | 6513 | −0.05 | 4.11 | 4.264 | 4.435 | 4.228 | 4.332 | 4.281 | 0.143 | 0.118 |
| 201250_s_at | NM_006516 | SLC2A1 | 6513 | 0.021 | 7.2 | 7.214 | 7.324 | 7.287 | 7.164 | 7.264 | 0.096 | 0.005 |
| 201269_s_at | AB028991 | NUDCD3 | 23386 | −0.15 | 3.37 | 3.358 | 3.431 | 3.287 | 3.082 | 3.236 | −0.002 | −0.202 |
| 201270_x_at | NM_015332 | NUDCD3 | 23386 | −0.25 | 7.7 | 7.591 | 7.68 | 7.625 | 7.097 | 7.324 | 0.008 | −0.433 |
| 201301_s_at | BC000182 | ANXA4 | 307 | −0.35 | 9.52 | 9.515 | 9.416 | 9.548 | 9.659 | 9.577 | −0.037 | 0.099 |
| 201302_at | NM_001153 | ANXA4 | 307 | −0.77 | 8.27 | 8.234 | 8.166 | 8.182 | 7.587 | 7.459 | −0.078 | −0.729 |
| 201326_at | BE737030 | CCT6A | 908 | 0.212 | 9.09 | 9.026 | 8.95 | 9.083 | 8.89 | 9.186 | −0.044 | −0.022 |
| 201327_s_at | NM_001762 | CCT6A | 908 | −0.06 | 10.3 | 10.38 | 10.31 | 10.4 | 10.1 | 10.13 | −0.009 | −0.248 |
| 201331_s_at | BC004973 | STAT6 | 6778 | 0.146 | 5.27 | 5.205 | 5.493 | 5.389 | 5.942 | 5.931 | 0.206 | 0.701 |
| 201332_s_at | NM_003153 | STAT6 | 6778 | 0.029 | 3.32 | 3.24 | 3.489 | 3.569 | 3.493 | 3.341 | 0.247 | 0.135 |
| 201373_at | NM_000445 | PLEC1 | 5339 | 0.296 | 6.65 | 6.677 | 6.669 | 6.843 | 6.677 | 6.759 | 0.091 | [symbol] |
| 201379_s_at | NM_003288 | TPD52L2 | 7165 | −0.18 | 7.75 | 7.815 | 7.866 | 7.706 | 7.763 | 7.809 | 0.005 | |
| 201381_x_at | AF057356 | CACYBP | 27101 | −0.35 | 10.5 | 10.44 | 10.53 | 10.55 | 10.14 | 10.08 | 0.09 | |
| 201382_at | NM_014412 | CACYBP | 27101 | −0.23 | 3.87 | 3.718 | 3.827 | 3.944 | 3.97 | 3.721 | 0.094 | |
| 201388_at | NM_002809 | PSMD3 | 5709 | 0.155 | 7.16 | 7.161 | 7.133 | 7.184 | 7.202 | 7.348 | 2E−04 | |
| 201400_at | NM_002795 | PSMB3 | 5691 | 0.128 | 9.83 | 9.783 | 9.844 | 9.772 | 9.799 | 9.865 | 0.004 | |
| 201401_s_at | M80776 | ADRBK1 | 156 | 0.002 | 4.16 | 4.019 | 4.257 | 4.289 | 4.64 | 4.708 | 0.185 | |
| 201402_at | NM_001619 | ADRBK1 | 156 | −0.25 | 4.55 | 4.161 | 4.193 | 4.165 | 3.85 | 3.912 | −0.179 | |
| 201423_s_at | AL037208 | CUL4A | 8451 | 0.247 | 6.01 | 5.739 | 6.102 | 5.99 | 6.406 | 6.558 | 0.171 | |
| 201424_s_at | NM_003589 | CUL4A | 8451 | 0.04 | 6.5 | 6.753 | 6.486 | 6.458 | 6.779 | 6.951 | −0.156 | |
| 201491_at | NM_012111 | AHSA1 | 10598 | 0.035 | 9.91 | 9.94 | 9.97 | 9.942 | 9.447 | 9.572 | 0.032 | |
| 201559_s_at | AF109196 | CLIC4 | 25932 | −0.19 | 6.44 | 6.737 | 6.461 | 6.513 | 6.47 | 6.515 | −0.104 | |
| 201560_at | NM_013943 | CLIC4 | 25932 | 0.171 | 8.6 | 8.63 | 8.568 | 8.547 | 8.593 | 8.538 | −0.056 | |
| 201564_s_at | NM_003088 | FSCN1 | 6624 | 0.331 | 4.54 | 3.674 | 3.989 | 3.442 | 3.846 | 4.179 | −0.393 | |
| 201578_at | NM_005397 | PODXL | 5420 | 0.517 | 8.08 | 8.381 | 8.329 | 8.334 | 8.289 | 7.889 | 0.102 | |
| 201605_x_at | NM_004368 | CNN2 | 1265 | −0.3 | 4.26 | 3.517 | 3.872 | 3.672 | 3.716 | 3.841 | −0.117 | |
| 201621_at | NM_005380 | NBL1 | 4681 | −0.18 | 4.55 | 4.174 | 4.339 | 4.859 | 4.517 | 4.561 | 0.237 | |
| 201623_s_at | BC000629 | DARS | 1615 | −0.19 | 10.6 | 10.6 | 10.57 | 10.6 | 10.76 | 10.66 | −0.017 | |
| 201624_at | NM_001349 | DARS | 1615 | −0.1 | 7.98 | 8.183 | 8.149 | 8.247 | 8.216 | 7.92 | 0.115 | |
| 201635_s_at | AI990766 | FXR1 | 8087 | −0.81 | 8.15 | 8.334 | 8.09 | 8.176 | 7.898 | 7.866 | −0.109 | |
| 201636_at | BG025078 | FXR1 | 8087 | −0.65 | 7.74 | 7.549 | 7.59 | 7.473 | 7.441 | 7.24 | −0.111 | |
| 201637_s_at | NM_005087 | FXR1 | 8087 | −0.57 | 9.07 | 9.085 | 8.989 | 8.839 | 8.776 | 8.747 | −0.163 | |
| 201638_s_at | BE676642 | CPSF1 | 29894 | −0.19 | 3.23 | 3.396 | 3.275 | 3.405 | 3.358 | 3.038 | 0.028 | |
| 201639_s_at | NM_013291 | CPSF1 | 29894 | 0.446 | 6.68 | 6.827 | 6.831 | 6.919 | 7.26 | 7.068 | 0.119 | |
| 201642_at | NM_005534 | IFNGR2 | 3460 | −0.04 | 6.3 | 6.143 | 6.24 | 6.39 | 6.193 | 6.291 | 0.091 | |
| 201643_x_at | NM_016604 | JMJD1B | 51780 | −0.15 | 6.42 | 6.626 | 6.404 | 6.526 | 6.527 | 6.761 | −0.059 | |
| 201654_s_at | AI991033 | HSPG2 | 3339 | −0.05 | 2.91 | 2.989 | 2.778 | 2.809 | 2.834 | 2.758 | −0.155 | |
| 201655_s_at | M85289 | HSPG2 | 3339 | 0.041 | 4.69 | 4.116 | 4.266 | 4.162 | 4.656 | 4.434 | −0.19 | 0.141 |
| 201688_s_at | BG389015 | TPD52 | 7163 | 0.609 | 8.95 | 9.006 | 8.847 | 8.871 | 8.613 | 8.552 | −0.121 | −0.398 |
| 201689_s_at | BE974098 | TPD52 | 7163 | 0.661 | 9.18 | 9.244 | 9.236 | 9.036 | 8.621 | 8.504 | −0.074 | −0.649 |
| 201690_s_at | AA524023 | TPD52 | 7163 | 0.876 | 10.3 | 10.28 | 10.16 | 10.16 | 9.877 | 9.859 | −0.107 | −0.397 |
| 201691_s_at | NM_005079 | TPD52 | 7163 | 0.051 | 3.7 | 3.762 | 3.615 | 3.691 | 3.226 | 3.396 | −0.078 | −0.42 |
| 201711_x_at | AI681120 | RANBP2 | 5903 | −0.17 | 7.48 | 7.483 | 7.488 | 7.523 | 7.412 | 7.374 | 0.026 | −0.087 |
| 201712_s_at | NM_006267 | RANBP2 | 5903 | 0.303 | 6.17 | 6.301 | 6.453 | 6.298 | 6.363 | 6.261 | 0.14 | 0.077 |
| 201713_s_at | D42063 | RANBP2 | 5903 | −0.11 | 7.83 | 7.88 | 7.791 | 7.888 | 7.39 | 7.648 | −0.014 | −0.334 |
| 201717_at | NM_004927 | MRPL49 | 740 | 0.374 | 9.17 | 9.139 | 9.151 | 9.275 | 9.343 | 9.385 | 0.06 | 0.211 |
| 201751_at | NM_014876 | JOSD1 | 9929 | 0.493 | 6.49 | 6.65 | 6.682 | 6.667 | 6.585 | 6.677 | 0.105 | 0.061 |
| 201772_at | NM_015878 | AZIN1 | 51582 | 0.053 | 8.95 | 8.835 | 8.855 | 8.741 | 8.829 | 8.858 | −0.084 | −0.038 |
| 201841_s_at | NM_001540 | HSPB1 | 3315 | −0.19 | 12.5 | 12.55 | 12.47 | 12.54 | 12.38 | 12.33 | −0.039 | −0.187 |
| 201842_s_at | AI826799 | EFEMP1 | 2202 | −0.39 | 5.94 | 5.809 | 6.155 | 6.08 | 6.922 | 6.744 | 0.243 | 0.959 |
| 201843_s_at | NM_004105 | EFEMP1 | 2202 | −0.81 | 3.68 | 3.453 | 3.855 | 3.939 | 4.008 | 4.171 | 0.33 | 0.523 |
| 201853_s_at | NM_021873 | CDC258 | 994 | −0.13 | 7.53 | 7.382 | 7.582 | 7.839 | 7.48 | 7.446 | 0.257 | 0.009 |
| 201913_s_at | NM_025233 | COASY | 80347 | 0.24 | 6.09 | 6.281 | 6.169 | 6.16 | 6.429 | 6.546 | −0.02 | 0.302 |
| 201922_at | NM_014886 | TINP1 | 10412 | −0.17 | 10.7 | 10.62 | 10.52 | 10.62 | 10.77 | 10.72 | −0.07 | 0.102 |
| 201971_s_at | NM_001690 | ATP6V1A | 523 | −0.77 | 6.31 | 6.15 | 6.129 | 5.85 | 6.301 | 6.435 | −0.242 | 0.136 |
| 201972_at | AF113129 | ATP6V1A | 523 | −0.32 | 9.67 | 9.639 | 9.68 | 9.603 | 9.909 | 9.925 | −0.014 | 0.261 |
| 201983_s_at | AW157070 | EGFR | 1956 | −0.44 | 4.36 | 4.435 | 4.676 | 4.468 | 4.964 | 4.916 | 0.172 | 0.54 |
| 201984_s_at | NM_005228 | EGFR | 1956 | −0.39 | 4.21 | 4.118 | 3.99 | 4.121 | 4.365 | 4.114 | −0.107 | 0.077 |
| 201994_at | NM_012286 | MORF4L2 | 9643 | −0.08 | 11.6 | 11.58 | 11.53 | 11.57 | 11.38 | 11.45 | −0.048 | −0.184 |
| 202043_s_at | NM_004595 | SMS | 6611 | 0.197 | 8.44 | 8.365 | 8.305 | 8.24 | 8.043 | 7.95 | −0.131 | −0.407 |
| 202055_at | AA652173 | KPNA1 | 3836 | 0.056 | 7.26 | 7.134 | 7.205 | 7.164 | 7.058 | 7.073 | −0.011 | −0.13 |
| 202056_at | AW051311 | KPNA1 | 3836 | 0.17 | 6.85 | 6.674 | 6.821 | 6.906 | 6.299 | 6.593 | 0.103 | −0.314 |
| 202057_at | BC002374 | KPNA1 | 3836 | −0.17 | 4.97 | 4.995 | 5.013 | 4.896 | 5.196 | 5.498 | −0.029 | 0.363 |
| 202058_s_at | BC002374 | KPNA1 | 3836 | −0.12 | 6.91 | 6.933 | 6.863 | 6.749 | 6.844 | 6.922 | −0.116 | −0.039 |
| 202059_s_at | NM_002264 | KPNA1 | 3836 | 0.243 | 7.72 | 7.736 | 7.633 | 7.524 | 7.921 | 7.649 | −0.151 | 0.055 |
| 202067_s_at | AI861942 | LDLR | 3949 | 0.561 | 6.73 | 6.732 | 6.62 | 6.69 | 6.493 | 6.655 | −0.078 | −0.159 |
| 202068_s_at | NM_000527 | LDLR | 3949 | 0.809 | 8.48 | 8.231 | 8.398 | 8.407 | 8.382 | 8.512 | 0.044 | 0.089 |
| 202104_s_at | NM_003319 | SPG7 | 6687 | −0.18 | 6.66 | 6.685 | 6.692 | 6.662 | 6.814 | 7.002 | 0.002 | 0.233 |
| 202106_at | NM_005895 | GOLGA3 | 2802 | 0.395 | 5.87 | 5.863 | 6.183 | 6.119 | 6.554 | 6.555 | 0.282 | 0.686 |
| 202151_s_at | NM_016172 | UBAC1 | 10422 | −0.22 | 7.83 | 7.845 | 7.744 | 7.753 | 7.898 | 7.928 | −0.088 | 0.076 |
| 202161_at | NM_002741 | PKN1 | 5585 | 0.429 | 3.37 | 3.606 | 3.514 | 3.701 | 3.383 | 3.807 | 0.118 | 0.106 |
| 202181_at | NM_014734 | KIAA0247 | 9766 | −0.25 | 5.32 | 5.447 | 5.302 | 5.69 | 6.122 | 6.142 | 0.111 | 0.747 |
| 202258_s_at | U50532 | N4BP2L2 | 10443 | 0.07 | 8.99 | 9.054 | 9.093 | 9.08 | 9.234 | 9.304 | 0.066 | 0.148 |
| 202259_s_at | NM_014887 | N4BP2L2 | 10443 | 0.12 | 5.4 | 5.023 | 5.009 | 4.823 | 5.27 | 5.127 | −0.297 | −0.014 |
| 202273_at | NM_002609 | PDGFRB | 5159 | −0.34 | 3.42 | 3.501 | 3.362 | 3.406 | 3.369 | 3.406 | −0.075 | −0.071 |
| 202301_s_at | BE396879 | RSRC2 | 65117 | 0.448 | 8.81 | 8.526 | 8.64 | 8.54 | 8.801 | 8.714 | −0.081 | 0.088 |
| 202302_s_at | NM_023032 | RSRC2 | 65117 | 0.42 | 9.15 | 9.114 | 9.087 | 9.004 | 8.992 | 9.123 | −0.08 [symbol] | |
| 202333_s_at | AA877765 | UBE2B | 7320 | −0.15 | 9.42 | 9.373 | 9.364 | 9.359 | 9.292 | 9.405 | −0.03 [symbol] | |
| 202334_s_at | AI768723 | UBE2B | 7320 | 0.143 | 7.51 | 7.538 | 7.484 | 7.393 | 7.515 | 7.599 | −0.08 [symbol] | |
| 202335_s_at | NM_003337 | UBE2B | 7320 | −0.21 | 2.55 | 2.643 | 2.473 | 2.46 | 2.611 | 2.604 | −0.12 [symbol] | |
| 202350_s_at | NM_002380 | MATN2 | 4147 | 0.645 | 5.95 | 5.728 | 5.859 | 5.806 | 6.238 | 6.236 | −0.00 [symbol] | |
| 202354_s_at | AW190445 | GTF2F1 | 2962 | −0.13 | 5.97 | 5.866 | 6.125 | 6.192 | 6.695 | 6.719 | 0.23 [symbol] | |
| 202355_s_at | BC000120 | GTF2F1 | 2962 | −0.44 | 5.88 | 5.947 | 6.08 | 6.097 | 6.455 | 6.58 | 0.17 [symbol] | |
| 202356_s_at | NM_002096 | GTF2F1 | 2962 | −0.51 | 6.01 | 5.818 | 6.199 | 5.874 | 6.255 | 6.298 | 0.12 [symbol] | |
| 202363_at | AF231124 | SPOCK1 | 6695 | 0.508 | 3.45 | 3.667 | 3.842 | 3.93 | 4.301 | 4.313 | −0.32 [symbol] | |
| 202367_at | NM_001913 | CUX1 | 1523 | −0.19 | 6.54 | 6.566 | 6.654 | 6.646 | 6.647 | 6.698 | 0.09 [symbol] | |
| 202393_s_at | NM_005655 | KLF10 | 7071 | 0.266 | 8.76 | 8.905 | 8.529 | 8.849 | 8.927 | 8.861 | 0.05 [symbol] | |
| 202397_at | NM_005796 | NUTF2 | 10204 | 0.464 | 8.65 | 8.718 | 8.675 | 8.717 | 8.692 | 8.822 | 0.0 [symbol] | |
| 202402_s_at | NM_001751 | CARS | 833 | 0.553 | 6.06 | 6.304 | 6.485 | 6.089 | 6.531 | 6.557 | 0.10 [symbol] | |
| 202405_at | BF432332 | TIAL1 | 7073 | 0.22 | 5.29 | 5.479 | 5.184 | 5.309 | 5.267 | 5.586 | −0.13 [symbol] | |
| 202406_s_at | NM_003252 | TIAL1 | 7073 | −0.15 | 9.65 | 5.553 | 9.623 | 9.653 | 9.371 | 9.459 | 0.03 [symbol] | |
| 202415_s_at | NM_012267 | HSPBP1 | 23640 | 0.395 | 6.03 | 5.923 | 6.018 | 6.107 | 6.377 | 6.351 | 0.08 [symbol] | |
| 202424_at | NM_030662 | MAPZK2 | 5605 | 0.195 | 6.28 | 6.358 | 6.268 | 6.197 | 6.613 | 6.443 | −0.08 [symbol] | |
| 202426_s_at | BE675800 | RXRA | 6256 | −0.18 | 4.19 | 3.911 | 3.987 | 4.082 | 4.463 | 4.445 | −0.01 [symbol] | |
| 202438_x_at | BF346014 | IDS | 3423 | 0.301 | 3.84 | 3.884 | 4.351 | 4.329 | 4.281 | 4.407 | 0.47 [symbol] | |
| 202439_s_at | NM_000202 | IDS | 3423 | 0.04 | 7.64 | 7.418 | 7.694 | 7.817 | 7.983 | 8.091 | 0.22 [symbol] | |
| 202449_s_at | NM_002957 | RXRA | 6256 | −0.15 | 7.94 | 7.907 | 7.756 | 7.971 | 8.255 | 8.155 | −0.06 [symbol] | |
| 202555_s_at | NM_005965 | MYLK | 4638 | 0.543 | 4.18 | 4.177 | 3.81 | 4.413 | 4.238 | 4.284 | −0.06 [symbol] | |
| 202575_at | NM_001878 | CRABP2 | 1382 | −0.05 | 8.55 | 8.43 | 8.427 | 8.42 | 8.402 | 8.511 | −0.06 [symbol] | |
| 202579_x_at | NM_006353 | HMGN4 | 10473 | 0.121 | 9.47 | 9.224 | 9.335 | 9.281 | 9.205 | 9.231 | −0.03 [symbol] | |
| 202586_at | AA772747 | POLR2L | 5441 | −0.04 | 4.76 | 4.554 | 4.383 | 4.373 | 5.253 | 5.212 | −0.27 [symbol] | |
| 202598_at | NM_005979 | S100A13 | 6284 | 0.082 | 9.27 | 9.307 | 9.236 | 9.229 | 9.341 | 9.336 | −0.05 [symbol] | |
| 202605_at | NM_000181 | GUSB | 2990 | −0.26 | 9.36 | 9.346 | 9.281 | 9.377 | 9.518 | 9.547 | −0.023 | 0.181 |
| 202615_at | BF222895 | GNAQ | 2776 | −0.24 | 8.55 | 8.5 | 8.497 | 8.607 | 8.602 | 8.573 | 0.26 | 0.061 |
| 202639_s_at | AI689052 | RANBP3 | 8498 | 0.31 | 4.68 | 4.902 | 4.424 | 4.84 | 4.728 | 4.873 | −0.16 | 0.009 |
| 202640_s_at | NM_003624 | RANBP3 | 8498 | 0.57 | 4.93 | 5.322 | 4.897 | 5.181 | 5.078 | 4.947 | −0.087 | −0.114 |
| 202671_s_at | NM_003681 | PDXK | 8566 | 0.754 | 8.5 | 8.474 | 8.558 | 8.565 | 8.935 | 8.893 | 0.072 | 0.425 |
| 202672_s_at | NM_001674 | AAATF3 | 467 | 0.86 | 3.42 | 3.544 | 3.937 | 3.696 | 3.983 | 3.493 | 0.334 | 0.255 |
| 202716_at | NM_002827 | PTPN1 | 5770 | −0.28 | 7.91 | 7.796 | 7.658 | 8.07 | 7.754 | 7.685 | 0.13 | −0.131 |
| 202733_at | NM_004199 | P4HA2 | 8974 | −0.14 | 7.81 | 7.859 | 7.616 | 7.741 | 8.309 | 8.332 | −0.157 | 0.485 |
| 202736_s_at | AA112507 | LSM4 | 25804 | −0.04 | 9.51 | 9.477 | 9.489 | 9.516 | 9.488 | 9.517 | 0.009 | 0.009 |
| 202737_s_at | NM_012321 | LSM4 | 25804 | −0.14 | 8.54 | 9.036 | 9.009 | 9.019 | 8.791 | 8.67 | 0.224 | 0.039 |
| 202740_at | NM_000666 | ACY1 | 95 | 0.139 | 6.45 | 6.658 | 6.35 | 6.59 | 6.872 | 6.815 | −0.087 | 0.286 |
| 207255_s_at | AI354854 | GPC1 | 2817 | 0.201 | 3.62 | 3.021 | 3.473 | 3.318 | 3.433 | 3.24 | 0.076 | 0.017 |
| 202756_s_at | NM_002081 | GPC1 | 2817 | −0.1 | 5.01 | 4.755 | 4.956 | 4.583 | 4.975 | 4.952 | −0.114 | 0.079 |
| 202759_s_at | BE879367 | AKAP2 /// PALM2 /// | 11217 /// 114299 /// | 0.625 | 3.8 | 3.314 | 3.558 | 3.8 | 3.261 | 3.59 | 0.123 | −0.13 |
| | | PALM2-AKAP2 | 445815 | | | | | | | | | |
| 202760_s_at | NM_007203 | PALM2-AKAP2 | 445815 | 0.95 | 3.07 | 2.974 | 3.089 | 3.149 | 3.166 | 3.18 | 0.095 | 0.15 |
| 202761_s_at | NM_015180 | SYNE2 | 23224 | −0.62 | 6.65 | 6.613 | 6.561 | 6.694 | 6.215 | 6.392 | −0.004 | −0.328 |
| 202797_at | NM_014016 | SACM1L | 22908 | −0.64 | 7.7 | 7.733 | 7.836 | 7.737 | 6.779 | 6.825 | 0.068 | −0.916 |
| 202806_at | NM_004395 | DBN1 | 1627 | 0.423 | 5.93 | 6.112 | 6.16 | 6.06 | 6.034 | 6.207 | 0.087 | 0.098 |
| 202833_s_at | NM_000295 | SEPINA1 | 5265 | −0.87 | 5.31 | 5.898 | 5.528 | 5.209 | 5.612 | 5.31 | −0.235 | −0.143 |
| 202865_at | AI695173 | DNAJB12 | 54788 | −0.04 | 3.66 | 3.951 | 3.944 | 3.642 | 3.905 | 3.747 | −0.012 | 0.021 |
| 202866_at | BG283782 | DNAJB12 | 54788 | 0.037 | 6.76 | 6.792 | 6.937 | 6.91 | 7.034 | 7.008 | 0.145 | 0.243 |
| 202867_s_at | NM_017626 | DNAJB12 | 54788 | −0.28 | 6.6 | 6.841 | 6.594 | 6.613 | 6.499 | 6.514 | −0.117 | −0.214 |
| 202905_x_at | AI796269 | NBN | 4683 | 0.161 | 10.1 | 10.12 | 10.16 | 10.21 | 10.14 | 10.06 | 0.069 | −0.019 |
| 202906_s_at | AP049895 | NBN | 4683 | 0.583 | 9.04 | 8.997 | 9.01 | 9.085 | 9.325 | 9.212 | 0.029 | 0.25 |
| 202907_s_at | NM_002485 | NBN | 4683 | −0.04 | 8.84 | 8.989 | 8.956 | 8.91 | 8.487 | 8.488 | 0.016 | −0.429 |
| 202918_s_at | AF151853 | MOBKL3 | 25843 | −0.03 | 10.4 | 10.32 | 10.3 | 10.34 | 10.34 | 10.3 | −0.021 | −0.02 |
| 202919_at | NM_015387 | MOBKL3 | 25843 | −0.12 | 8.42 | 8.24 | 8.339 | 8.138 | 7.979 | 8.065 | −0.09 | −0.306 |
| 202934_at | AI761561 | HK2 | 3099 | 0.884 | 5.8 | 5.99 | 5.969 | 5.931 | 5.985 | 5.895 | 0.057 | 0.047 |
| 202950_at | NM_001889 | CRYZ | 1429 | −1.35 | 7.35 | 7.478 | 7.503 | 7.305 | 7.052 | 6.776 | −0.009 | −0.499 |
| 202996_at | NM_021173 | POLD4 | 57804 | −0.25 | 7.52 | 7.5 | 7.567 | 7.578 | 7.757 | 7.617 | 0.062 | 0.176 |
| 203020_at | NM_014857 | RABGAP1L | 9910 | −0.2 | 6.64 | 6.694 | 6.631 | 6.69 | 6.581 | 6.741 | −0.008 | −0.007 |
| 203038_at | NM_002844 | PTPRK | 5796 | 0.738 | 9.56 | 9.655 | 9.479 | 9.512 | 9.944 | 10.01 | −0.11 | 0.374 |
| 203051_at | NM_014952 | BAHD1 | 22893 | −0.15 | 4.58 | 4.209 | 4.413 | 4.202 | 4.236 | 4.628 | −0.086 | 0.039 |
| 203064_s_at | NM_004514 | FOXK2 | 3607 | 0.529 | 5.45 | 5.544 | 5.654 | 5.675 | 5.997 | 6.209 | 0.168 | 0.607 |
| 203081_at | NM_020248 | CTNNBIP1 | 56998 | −0.48 | 4.04 | 4.922 | 4.724 | 4.472 | 5.004 | 4.57 | 0.117 | 0.307 |
| 203082_at | NM_014753 | BMS1 | 9790 | 0.191 | 6.72 | 6.693 | 6.858 | 6.722 | 6.653 | 6.804 | 0.082 | 0.02 |
| 203107_x_at | NM_002952 | RPS2 | 6187 | −0.08 | 13.1 | 13.2 | 13.2 | 13.16 | 13.11 | 13.13 | 0.012 | −0.048 |
| 203113_s_at | NM_001960 | EEF1D | 1936 | −0.13 | 10.5 | 10.49 | 10.49 | 10.48 | 10.38 | 10.42 | 0.012 | −0.079 |
| 203173_s_at | AW080196 | C16orf62 | 57020 | −0.45 | 5.96 | 6.301 | 6.231 | 6.168 | 6.419 | 6.331 | 0.0 [symbol] | |
| 203179_at | NM_000155 | GALT | 2592 | −0.66 | 4.56 | 4.321 | 4.277 | 4.322 | 4.429 | 4.369 | −0.14 [symbol] | |
| 203188_at | NM_006876 | B3GNT1 | 11041 | −0.02 | 6.42 | 6.328 | 6.04 | 6.208 | 7.9 | 7.905 | −0.24 [symbol] | |
| 203193_at | NM_004451 | ESRRA | 2101 | 0.15 | 4.33 | 4.533 | 4.443 | 4.515 | 4.547 | 4.416 | 0.04 [symbol] | |
| 203231_s_at | AW235612 | ATXN1 | 6310 | 0.025 | 5.92 | 5.981 | 5.792 | 5.555 | 5.684 | 5.621 | −0.27 [symbol] | |
| 203232_s_at | NM_000332 | ATXN1 | 6310 | 0.084 | 7.56 | 7.726 | 7.477 | 7.501 | 7.136 | 7.046 | −0.15 [symbol] | |
| 203234_at | NM_003364 | UPP1 | 7378 | 1.984 | 3.14 | 3.251 | 3.302 | 3.351 | 3.263 | 3.431 | 0.13 [symbol] | |
| 203258_at | NM_006442 | DRAP1 | 10589 | 0.544 | 6.96 | 7.081 | 7.049 | 7.035 | 7.328 | 7.294 | 0.02 [symbol] | |
| 203297_s_at | BG029530 | JARID2 | 3720 | 0.54 | 7.74 | 7.598 | 7.588 | 7.626 | 7.506 | 7.524 | −0.06 [symbol] | |
| 203298_s_at | NM_004973 | JARID2 | 3720 | 0.729 | 8.43 | 8.341 | 8.351 | 8.37 | 8.253 | 8.224 | −0.02 [symbol] | |
| 203321_s_at | AK022588 | ADNP2 | 22850 | −0.17 | 7.55 | 7.409 | 7.583 | 7.647 | 7.443 | 7.385 | 0.13 [symbol] | |
| 203322_at | AU145934 | ADNP2 | 22850 | −0.04 | 6.34 | 6.153 | 6.392 | 6.556 | 6.087 | 6.074 | 0.22 [symbol] | |
| 203323_at | BF197655 | CAV2 | 858 | 0.236 | 4.81 | 4.903 | 4.933 | 4.802 | 6.006 | 5.831 | 0.01 [symbol] | |
| 203324_s_at | NM_001233 | CAV2 | 858 | 0.305 | 6.84 | 7.054 | 7.218 | 7.063 | 7.773 | 7.622 | 0.1 [symbol] | |
| 203334_at | NM_004941 | DHX8 | 1659 | −0.23 | 5.82 | 5.878 | 5.783 | 5.836 | 5.944 | 6.093 | −0.0 [symbol] | |
| 203366_at | NM_002693 | POLG | 5428 | 0.602 | 7.12 | 7.246 | 7.35 | 7.422 | 7.543 | 7.53 | 0.20 [symbol] | |
| 203368_at | NM_015513 | CRELD1 | 7898 | −0.59 | 4.09 | 4.126 | 4.16 | 4.156 | 4.08 | 4.146 | 0.05 [symbol] | |
| 203406_at | NM_005926 | MFAF1 | 4236 | −0.24 | 8.38 | 8.296 | 8.507 | 8.42 | 8.301 | 8.237 | 0.12 [symbol] | |
| 203456_at | NM_007213 | PRAF2 | 11230 | −0.09 | 6.56 | 6.504 | 6.464 | 6.564 | 7.178 | 7.144 | −0.01 [symbol] | |
| 203458_at | AI951454 | SPR | 6697 | −0.62 | 7.59 | 7.72 | 7.787 | 7.699 | 7.596 | 7.523 | 0.08 [symbol] | |
| 203499_at | NM_004431 | EPHA2 | 1969 | 0.946 | 3.23 | 3.643 | 3.425 | 3.718 | 4.459 | 4.428 | 0.13 [symbol] | |
| 203511_s_at | AF041432 | TRAPPC3 | 27095 | 0.106 | 8.04 | 7.991 | 8.062 | 7.957 | 8.123 | 8.018 | −0.00 [symbol] | |
| 203512_at | NM_014408 | TRAPPC3 | 27095 | 0.142 | 6.94 | 6.93 | 7.042 | 6.975 | 6.953 | 6.925 | 0.07 [symbol] | |
| 203515_s_at | NM_006556 | PMVK | 10654 | −0.09 | 7.01 | 6.907 | 6.943 | 7.027 | 7.04 | 7 | 0.02 [symbol] | |
| 203557_s_at | NM_000281 | PCBD1 | 5092 | −0.33 | 8 | 8.07 | 8.08 | 8.109 | 8.216 | 8.138 | 0.05 [symbol] | |
| 203561_at | NM_021642 | FCGR2A | 2212 | −0.08 | 2.87 | 2.84 | 2.942 | 2.904 | 2.664 | 2.8 | 0.06 [symbol] | |
| 203571_s_at | NM_006829 | C10orf116 | 10974 | −0.82 | 8.62 | 8.71 | 8.545 | 8.618 | 8.744 | 8.739 | −0.085 | 0.075 |
| 203627_at | AI830598 | IGF3R | 3480 | −0.17 | 9.04 | 8.865 | 9.009 | 8.985 | 8.944 | 8.973 | 0.047 | 0.008 |
| 203628_at | H05812 | IGF1R | 3480 | −0.58 | 8.55 | 8.158 | 8.584 | 8.739 | 8.222 | 8.093 | 0.308 | −0.195 |
| 203710_at | NM_002222 | ITPR1 | 3708 | 0.097 | 4.75 | 4.8 | 4.771 | 4.648 | 5.342 | 5.213 | −0.068 | 0.4 |
| 203778_at | NM_005908 | MANBA | 4126 | 0.167 | 4.57 | 5.012 | 4.826 | 4.639 | 4.858 | 4.916 | −0.061 | 0.093 |
| 203792_x_at | BC004558 | PCGF2 | 7703 | 0.18 | 4.26 | 4.614 | 4.212 | 4.501 | 4.826 | 4.624 | −0.081 | 0.287 |
| 203793_x_at | NM_007144 | PCGF2 | 7703 | −0.1 | 4.23 | 3.921 | 4.136 | 3.949 | 4.14 | 4.079 | −0.034 | 0.033 |
| 203810_at | BG252490 | DNA3B4 | 11080 | −0.34 | 4 | 4.487 | 4.415 | 4.122 | 4.155 | 4.311 | 0.025 | −0.011 |
| 203811_s_at | NM_007034 | DNAJB4 | 11080 | −0.34 | 4.87 | 4.952 | 5.009 | 4.78 | 4.865 | 4.903 | −0.016 | −0.026 |
| 203818_s_at | NM_006802 | SF3A3 | 10946 | 0.098 | 6.22 | 6.365 | 6.358 | 6.511 | 6.345 | 6.243 | 0.141 | 3E−04 |
| 203830_at | NM_022344 | C17orf75 | 64149 | −0.19 | 5.87 | 6.167 | 6.022 | 5.935 | 5.926 | 5.989 | −0.042 | −0.063 |
| 203860_at | NM_000282 | PCCA | 5095 | −0.24 | 5.92 | 5.807 | 5.846 | 5.942 | 6.144 | 6.205 | 0.029 | 0.309 |
| 203876_s_at | AI761713 | MMP11 | 4320 | −0.04 | 3.09 | 2.98 | 2.974 | 2.997 | 3.079 | 2.982 | −0.049 | −0.005 |
| 203877_at | NM_005940 | MMP11 | 4320 | 0.111 | 3.2 | 2.963 | 2.979 | 3.111 | 2.639 | 2.885 | −0.036 | −0.319 |
| 203878_s_at | NM_005940 | MMP11 | 4320 | −0.19 | 3.27 | 3.481 | 3.501 | 3.278 | 3.291 | 3.724 | 0.016 | 0.134 |
| 203886_s_at | NM_001998 | FBLN2 | 2199 | −0.05 | 2.89 | 2.865 | 2.748 | 3.115 | 3.016 | 2.934 | 0.055 | 0.098 |
| 203905_at | NM_002582 | PARN | 5073 | −0.31 | 8.34 | 8.212 | 8.269 | 8.146 | 7.842 | 7.896 | −0.067 | −0.406 |
| 203963_at | NM_001218 | CA12 | 771 | −0.39 | 9.79 | 9.67 | 9.521 | 9.563 | 9.542 | 9.695 | −0.189 | −0.112 |
| 203966_s_at | NM_021003 | PPM1A | 5494 | 0.01 | 8.52 | 8.512 | 8.358 | 8.412 | 8.548 | 8.723 | −0.133 | 0.118 |
| 203969_at | AU157140 | PEX3 | 8504 | 0.007 | 3.19 | 3.247 | 3.209 | 3.141 | 3.132 | 3.244 | −0.044 | −0.031 |
| 203970_s_at | NM_003630 | PEX3 | 8504 | −0.46 | 6.23 | 6.331 | 5.349 | 6.284 | 5.807 | 5.589 | 0.035 | −0.584 |
| 203972_s_at | AB035307 | PEX3 | 8504 | −0.2 | 7.43 | 7.398 | 7.29 | 7.447 | 6.765 | 6.81 | −0.044 | −0.625 |
| 204023_at | NM_002916 | RFC4 | 5984 | 0.064 | 9.51 | 9.579 | 9.655 | 9.635 | 8.485 | 8.497 | 0.1 | −1.055 |
| 204030_s_at | NM_014575 | SCHIP1 | 29970 | −0.03 | 2.83 | 3.287 | 3.216 | 3.095 | 2.978 | 3.103 | 0.097 | −0.018 |
| 204053_x_at | U96180 | PTEN | 5728 | −0.1 | 8.07 | 8.099 | 8.103 | 8.206 | 8.461 | 8.318 | 0.07 | 0.305 |
| 204054_at | NM_000314 | PTEN | 5728 | 0.08 | 4.31 | 4.157 | 4.208 | 3.991 | 4.509 | 4.515 | −0.133 | 0.279 |
| 204065_at | NM_004854 | CHST10 | 9486 | 0.006 | 3.77 | 3.734 | 3.85 | 3.983 | 4.137 | 3.943 | 0.166 | 0.29 |
| 204068_at | NM_006281 | STK3 | 6788 | 0.57 | 8.13 | 8.195 | 8.129 | 8.032 | 8.712 | 8.751 | −0.083 | 0.568 |
| 204095_s_at | AL521391 | ELL | 8178 | 0.465 | 3.34 | 3.341 | 3.252 | 3.062 | 3.764 | 3.571 | −0.182 | 0.329 |
| 204096_s_at | AL136771 | ELL | 8178 | 0.023 | 2.96 | 2.89 | 3.005 | 3.03 | 3.242 | 3.181 | 0.095 | 0.289 |
| 204163_at | NM_007046 | EMILIN1 | 11117 | 0 | 2.9 | 2.878 | 2.92 | 2.84 | 2.91 | 2.79 | −0.009 | −0.039 |
| 204170_s_at | NM_001827 | CKS2 | 1164 | −0.69 | 11.3 | 11.49 | 11.35 | 11.42 | 30.86 | 10.92 | −0.034 | −0.527 |
| 204173_at | NM_002475 | MYL6B | 140465 | −0.45 | 7.97 | 8.014 | 7.972 | 7.979 | 8.357 | 8.347 | −0.015 | 0.361 |
| 204190_at | NM_005800 | USPL1 | 10208 | 0.029 | 7.82 | 7.797 | 7.813 | 7.653 | 7.81 | 7.717 | −0.074 | −0.044 |
| 204202_at | NM_017604 | IQCE | 23288 | 0.068 | 4.76 | 4.661 | 4.71 | 5.215 | 5.29 | 5.638 | 0.253 | 0.754 |
| 204238_s_at | NM_006443 | C6orf108 | 10591 | −6.23 | 8.29 | 8.212 | 8.245 | 8.229 | 8.292 | 8.416 | −0.016 | 0.101 |
| 204292_x_at | NM_000455 | STK11 | 6794 | −0.04 | 3.52 | 3.862 | 3.62 | 3.775 | 4.047 | 4.026 | 0.006 | 0.345 |
| 204306_s_at | NM_004357 | CD151 | 977 | 0.02 | 6.75 | 6.876 | 6.913 | 6.945 | 6.756 | 6.791 | 0.116 | −0.039 |
| 204402_at | NM_012265 | RHBDD3 | 25807 | 0.252 | 3.77 | 3.729 | 3.761 | 3.661 | 3.513 | 3.681 | −0.04 | −0.154 |
| 204441_s_at | NM_002689 | POLA2 | 23649 | −0.12 | 7.23 | 6.929 | 7.065 | 7.151 | 6.201 | 6.189 | 0.03 | |
| 204442_x_at | NM_003573 | LTBP4 | 8425 | −0.22 | 3.97 | 4.043 | 3.801 | 3.925 | 4.405 | 4.102 | −0.14 | |
| 204503_at | NM_001988 | EVPL | 2125 | 0.013 | 3.91 | 3.713 | 3.995 | 4.078 | 3.64 | 4.287 | 0.22 | |
| 204508_s_at | BC001012 | CA12 | 771 | −0.13 | 6.23 | 6.434 | 5.868 | 6.51 | 6.125 | 6.448 | −0.14 | |
| 204509_at | NM_017689 | CA12 | 771 | 0.17 | 3.86 | 3.575 | 3.704 | 3.785 | 3.582 | 3.868 | 0.02 | |
| 204537_s_at | NM_004961 | GABRE | 2564 | 0.045 | 2.69 | 2.823 | 2.789 | 2.901 | 2.71 | 2.83 | 0.08 | |
| 204539_s_at | NM_014246 | CELSR1 | 9620 | −0.06 | 2.9 | 2.919 | 2.947 | 2.929 | 2.852 | 3.193 | 0.02 | |
| 204625_s_at | BF115658 | ITGB3 | 3690 | 0.116 | 3.05 | 3.064 | 3 | 2.873 | 3.111 | 3.185 | −0.12 | |
| 204626_s_at | J02703 | ITGB3 | 3690 | 0.082 | 3.3 | 3.227 | 3.145 | 3.156 | 3.056 | 3.181 | −0.11 | |
| 204627_s_at | M35999 | ITGB3 | 3690 | −0.1 | 2.74 | 2.694 | 2.565 | 2.452 | 2.713 | 2.656 | −0.20 | |
| 204628_s_at | NM_000212 | ITGB3 | 3690 | −0.01 | 2.84 | 3.057 | 2.896 | 2.957 | 2.956 | 3.095 | −0.0 | |
| 204691_x_at | NM_003560 | PLA2G6 | 8398 | −0.21 | 3.75 | 3.547 | 3.662 | 3.586 | 3.516 | 3.352 | −0.02 | |
| 204762_s_at | BE670563 | GNAO1 | 2775 | −0.12 | 3.03 | 2.661 | 2.94 | 2.837 | 2.809 | 3.009 | 0.04 | |
| 204763_s_at | NM_020988 | GNAO1 | 2775 | −0.18 | 3.25 | 3.072 | 3.327 | 3.301 | 3.366 | 3.209 | 0.15 | |
| 204773_at | NM_004512 | IL11RA | 3590 | −0.67 | 3.59 | 3.383 | 3.741 | 3.594 | 3.454 | 3.629 | −0.18 | |
| 204785_x_at | NM_000874 | IFNAR2 | 3455 | −0.3 | 6.26 | 6.114 | 5.925 | 6.08 | 5.78 | 5.654 | −0.18 | |
| 204786_s_at | L41944 | IFNAR2 | 3455 | 0.243 | 4.48 | 4.477 | 4.453 | 4.559 | 4.741 | 4.791 | 0.02 | |
| 204802_at | NM_004165 | RRAD | 6236 | −0.15 | 2.46 | 2.274 | 2.492 | 2.277 | 2.417 | 2.482 | 0.01 | |
| 204803_s_at | NM_004165 | RRAD | 6236 | −0.22 | 4.01 | 4.056 | 3.359 | 4.187 | 3.879 | 4.17 | −0.25 | |
| 204857_at | NM_003550 | MAD1L1 | 8379 | −0.07 | 6.31 | 6.771 | 6.556 | 6.807 | 7.108 | 7.009 | 0.14 | |
| 204883_s_at | AI968626 | HUS1 | 3364 | 0.388 | 7.19 | 7.157 | 7.232 | 7.277 | 7.327 | 7.292 | 0.08 | |
| 204884_s_at | NM_004507 | HUS1 | 3364 | −0.02 | 2.9 | 2.777 | 2.853 | 2.635 | 2.615 | 2.846 | −0.09 | |
| 204945_at | NM_002846 | PTPRN | 5798 | −0.06 | 2.72 | 2.711 | 2.702 | 2.772 | 2.67 | 2.757 | 0.02 | |
| 204962_s_at | NM_001809 | CENPA | 1058 | −0.24 | 8.68 | 8.607 | 3.635 | 8.583 | 8.304 | 8.345 | −0.03 | |
| 204981_at | NM_002555 | SLC22A18 | 5002 | −0.11 | 7.84 | 7.72 | 7.667 | 7.775 | 8.7 | 8.54 | −0.05 | |
| 204995_at | AL567411 | CDK5R1 | 8851 | 0.657 | 3.35 | 3.265 | 3.351 | 3.033 | 3.034 | 3.44 | −0.11 | |
| 204996_s_at | NM_003885 | CDK5R1 | 8851 | −0.17 | 2.75 | 2.765 | 2.746 | 2.816 | 2.645 | 2.624 | 0.023 | −0.123 |
| 205003_at | NM_014705 | DOCK4 | 9732 | 0.336 | 3.72 | 3.555 | 3.792 | 3.744 | 3.328 | 4.201 | 0.133 | 0.129 |
| 205005_s_at | AW293531 | NMT2 | 9397 | 0.117 | 5.19 | 4.959 | 5.178 | 5.272 | 4.983 | 5.052 | 0.153 | −0.055 |
| 205006_s_at | NM_004808 | NMT2 | 9397 | −0.03 | 4.49 | 4.715 | 4.747 | 4.603 | 4.227 | 3.911 | 0.075 | −0.532 |
| 205048_s_at | NM_003832 | PSPH | 5723 | −0.17 | 3.5 | 3.74 | 3.894 | 3.899 | 3.767 | 3.757 | 0.276 | 0.142 |
| 205089_at | NM_003416 | ZNF7 | 7553 | 0.511 | 6.93 | 6.858 | 7.05 | 7.055 | 7.085 | 7.187 | 0.158 | 0.242 |
| 205092_x_at | NM_014950 | ZBTB1 | 22890 | 0.255 | 4.11 | 3.861 | 3.878 | 3.896 | 3.974 | 3.739 | −0.099 | −0.129 |
| 205093_at | NM_014935 | PLEKHA6 | 22874 | −0.15 | 4.01 | 4.431 | 4.106 | 4.695 | 4.595 | 4.538 | 0.182 | 0.347 |
| 205133_s_at | NM_002157 | HSPE1 | 3336 | −0.56 | 10.7 | 10.65 | 10.5 | 10.49 | 9.947 | 10.03 | −0.184 | −0.691 |
| 205141_at | NM_001145 | ANG | 283 | −0.42 | 3.67 | 3.764 | 3.726 | 3.677 | 3.494 | 3.26 | −0.017 | −0.342 |
| 205158_at | NM_002937 | RNASE4 | 6038 | −0.69 | 3.26 | 3.403 | 2.914 | 3.428 | 3.036 | 3.073 | −0.162 | −0.278 |
| 205163_at | NM_013292 | MYLPF | 29895 | −0.22 | 3.3 | 3.594 | 3.212 | 3.414 | 3.396 | 3.179 | −0.133 | −0.159 |
| 205175_at | NM_000221 | KHK | 3795 | −0.05 | 3.38 | 3.358 | 3.141 | 3.417 | 3.093 | 3.112 | −0.089 | −0.265 |
| 205176_s_at | NM_014288 | ITGB3BP | 23421 | −0.46 | 7.7 | 7.608 | 7.703 | 7.832 | 7.524 | 7.132 | 0.114 | −0.326 |
| 205189_s_at | NM_000136 | FANCC | 2176 | −0.1 | 4.45 | 4.344 | 4.458 | 4.078 | 3.678 | 4.103 | −0.131 | −0.508 |
| 205194_at | NM_004577 | PSPH | 5723 | 0.33 | 6.58 | 6.649 | 6.987 | 6.799 | 6.446 | 6.298 | 0.278 | −0.242 |
| 205227_at | NM_002182 | IL1RAP | 3556 | −0.62 | 3.9 | 3.463 | 4.479 | 4.545 | 4.02 | 4.404 | 0.828 | 0.529 |
| 205263_at | AF082283 | BCL10 | 8915 | 0.117 | 7.41 | 7.533 | 7.673 | 7.586 | 7.552 | 7.495 | 0.156 | 0.05 |
| 205274_at | U87964 | GTPBP1 | 9567 | −0.25 | 3.18 | 3.027 | 2.954 | 3.171 | 3.065 | 3.107 | −0.041 | −0.018 |
| 205275_at | BE866976 | GTPBP1 | 9567 | 0.086 | 3.29 | 3.21 | 3.193 | 3.333 | 3.291 | 3.176 | 0.012 | −0.017 |
| 205276_s_at | NM_004286 | GTPBP1 | 9567 | −0.01 | 2.96 | 3.242 | 3.376 | 3.175 | 3.195 | 3.328 | 0.175 | 0.161 |
| 205292_s_at | NM_002137 | HNRNPA2B1 | 3181 | −0.17 | 11.3 | 11.36 | 11.26 | 11.28 | 11.06 | 10.94 | −0.078 | −0.348 |
| 205293_x_at | AB017120 | BAIAP2 | 10458 | 0.344 | 3.68 | 3.246 | 3.399 | 3.445 | 3.492 | 4.493 | −0.039 | 0.032 |
| 205294_at | NM_017450 | BAIAP2 | 10458 | 0.004 | 3.64 | 3.479 | 3.559 | 3.553 | 3.515 | 3.569 | −0.002 | −0.016 |
| 205320_at | NM_005883 | APC2 | 10297 | −0.02 | 3.19 | 2.855 | 2.908 | 3.295 | 3.47 | 3.132 | 0.080 | 0.281 |
| 205341_at | NM_014601 | EHD2 | 30846 | −0.05 | 3.35 | 2.994 | 3.376 | 3.168 | 3.374 | 3.542 | 0.101 | 0.286 |
| 205349_at | NM_002068 | GNA15 | 2769 | 0.716 | 4.41 | 4.957 | 4.831 | 4.348 | 4.544 | 4.208 | −0.093 | −0.306 |
| 205359_at | NM_004274 | AKAP6 | 9472 | 0.144 | 2.74 | 2.59 | 2.78 | 2.526 | 2.752 | 2.712 | −0.014 | 0.065 |
| 205411_at | NM_006282 | STK4 | 6789 | 0.415 | 2.98 | 3.05 | 2.934 | 3.061 | 3.313 | 3.132 | −0.017 | 0.209 |
| 205457_at | NM_024294 | C6orf106 | 64771 | −0.24 | 5.51 | 5.883 | 5.699 | 5.73 | 5.811 | 5.76 | 0.019 | 0.09 |
| 205463_at | NM_002607 | PDGFA | 5154 | 0.592 | 5.87 | 5.958 | 5.938 | 6.321 | 7.107 | 7.017 | 0.213 | 1.145 |
| 205485_at | NM_000540 | RYR1 | 6261 | −0.18 | 3.15 | 2.991 | 3.275 | 3.231 | 2.976 | 3.33 | 0.181 | 0.081 |
| 205543_at | NM_014278 | HSPA4L | 22824 | −0.36 | 6.42 | 6.497 | 6.188 | 6.453 | 6.021 | 6.013 | −0.137 | −0.441 |
| 205579_at | NM_000861 | HRH1 | 3269 | −0.05 | 2.96 | 3.458 | 3.262 | 3.612 | 4.046 | 3.073 | 0.23 | 0.353 |
| 205580_at | D28481 | HRH1 | 3269 | −0.05 | 3.19 | 3.345 | 3.184 | 3.135 | 3.041 | 3.252 | −0.109 | −0.122 |
| 205617_at | NM_000951 | PRRG2 | 5639 | −0.12 | 3.71 | 3.795 | 3.629 | 3.879 | 3.931 | 3.913 | 2E−04 | 0.168 |
| 205640_at | NM_000694 | ALDH3B1 | 221 | −0.54 | 3.67 | 3.638 | 3.39 | 3.39 | 3.71 | 3.521 | −0.262 | −0.036 |
| 205643_s_at | NM_004576 | PPP2R2B | 5521 | 0.035 | 3.38 | 3.01 | 3.114 | 3.177 | 3.134 | 3.002 | −0.049 | −0.127 |
| 205648_at | NM_003391 | WNT2 | 7472 | 0.067 | 3.71 | 3.576 | 3.769 | 3.696 | 3.536 | 3.982 | 0.091 | 0.117 |
| 205674_x_at | NM_001680 | FXYD2 | 486 | −0.15 | 3.13 | 3.321 | 2.988 | 3.21 | 3.296 | 3.363 | −0.12 | |
| 205687_at | NM_019116 | UBFD1 | 56061 | −0.14 | 8.44 | 8.414 | 8.342 | 8.369 | 8.492 | 8.425 | −0.0 | |
| 205724_at | NM_000299 | PKP1 | 5317 | 0.78 | 3.43 | 3.371 | 3.352 | 3.716 | 3.869 | 3.644 | 0.13 | |
| 205829_at | NM_000413 | HSD17B1 | 3292 | 1.076 | 3.23 | 3.687 | 3.452 | 3.582 | 3.977 | 3.649 | 0.05 | |
| 205858_at | NM_002507 | NGFR | 4804 | 0.1 | 2.97 | 2.789 | 2.878 | 2.837 | 2.821 | 2.936 | −0.02 | |
| 205872_x_at | NM_022359 | PDE4DIP | 9659 | 0.844 | 4.27 | 4.265 | 3.922 | 4.24 | 4.23 | 4.242 | −0.18 | |
| 205873_at | NM_004278 | PIGL | 9487 | 0.447 | 4.6 | 4.501 | 4.24 | 4.6 | 4.576 | 4.653 | −0.12 | |
| 205945_at | NM_000565 | IL6R | 3570 | 0.063 | 3.87 | 3.839 | 3.988 | 3.949 | 4.156 | 3.82 | 0.11 | |
| 205967_at | NM_003542 | HIST1H4A /// | 121504 /// 554313 | −0.2 | 9.92 | 10.08 | 9.958 | 10.24 | 9.19 | 9.314 | 0.09 | |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 206066_s_at | NM_002876 | RAD51C | 5889 | −0.21 | 9.05 | 9.023 | 9.199 | 9.241 | 8.812 | 8.677 | 0.18 | |
| 206105_at | NM_002025 | AFF2 | 2334 | −0.17 | 3.43 | 3.045 | 3.406 | 3.352 | 3.238 | 3.347 | 0.13 | |
| 206212_at | NM_001869 | CPA2 | 1358 | 0.127 | 3.27 | 3.256 | 3.198 | 3.345 | 3.187 | 3.097 | 0.00 | |
| 206219_s_at | NM_005428 | VAV1 | 7409 | 0.167 | 3.73 | 3.566 | 3.562 | 3.789 | 3.628 | 3.535 | 0.02 | |
| 206236_at | NM_005282 | GPR4 | 2828 | 0.051 | 2.8 | 3.074 | 3.02 | 3.162 | 3.23 | 2.998 | 0.152 | 0.175 |
| 206248_at | NM_005400 | PRKCE | 5581 | −0.27 | 3.43 | 3.662 | 3.398 | 3.457 | 3.254 | 3.655 | −0.12 | −0.093 |
| 206275_s_at | NM_014632 | MICAL2 | 9645 | 0.25 | 3.59 | 3.645 | 3.377 | 3.575 | 3.537 | 3.596 | −0.14 | −0.05 |
| 206316_s_at | NM_014708 | KNTC1 | 9735 | −0.23 | 7.2 | 7.306 | 7.343 | 7.433 | 6.46 | 6.489 | 0.133 | −0.78 |
| 206322_at | NM_003490 | SYN3 | 8224 | −0.23 | 3.35 | 3.2 | 3.253 | 3.183 | 3.263 | 3.114 | −0.057 | −0.087 |
| 206324_s_at | NM_014326 | DAPK2 | 23604 | 0.206 | 3.57 | 3.779 | 3.68 | 3.634 | 3.591 | 3.715 | −0.018 | −0.021 |
| 206342_x_at | NM_006123 | IDS | 3423 | 0.024 | 7.87 | 7.82 | 8.059 | 7.887 | 7.815 | 8.055 | 0.129 | 0.092 |
| 206357_at | NM_025136 | OPA3 | 80207 | 0.081 | 4.41 | 4.205 | 4.178 | 4.268 | 4.604 | 4.608 | −0.087 | 0.296 |
| 206400_at | NM_002307 | LGALS7 /// LGALS7B | 3963 /// 653499 | 0.169 | 3.81 | 3.663 | 3.672 | 3.346 | 3.219 | 3.913 | −0.226 | −0.17 |
| 206410_at | NM_021969 | NR0B2 | 8431 | 0.112 | 3.15 | 3.193 | 3.123 | 3.168 | 3.023 | 3.353 | −0.025 | 0.018 |
| 206452_x_at | NM_021131 | PPP2R4 | 5524 | 0.032 | 6.65 | 6.843 | 6.917 | 6.934 | 6.802 | 6.779 | 0.18 | 0.045 |
| 206492_at | NM_002012 | FHIT | 2272 | 0.297 | 3.16 | 2.872 | 3.068 | 2.996 | 3.177 | 2.982 | 0.017 | 0.065 |
| 206504_at | NM_000782 | CYP24A1 | 1591 | −0.2 | 3.38 | 3.238 | 3.136 | 3.368 | 3.125 | 3.134 | −0.058 | −0.18 |
| 206571_s_at | NM_004834 | MAP4K4 | 9448 | 0.136 | 5.1 | 5.156 | 5.086 | 4.751 | 5.325 | 5.346 | −0.208 | 0.21 |
| 206577_at | NM_003381 | VIP | 7432 | 0.163 | 2.59 | 2.754 | 2.705 | 2.576 | 2.523 | 2.59 | −0.032 | −0.116 |
| 206582_s_at | NM_005682 | GPRS6 | 9289 | 0.092 | 3.83 | 3.919 | 3.791 | 3.915 | 3.745 | 3.811 | −0.023 | −0.098 |
| 206709_x_at | NM_005309 | GPT | 2875 | −0.05 | 2.97 | 3.004 | 3.054 | 3.05 | 2.957 | 2.977 | 0.066 | −0.019 |
| 206720_at | NM_002410 | MGATS | 4249 | 0.213 | 2.96 | 3.252 | 3.015 | 2.838 | 3.078 | 3.06 | −0.18 | −0.037 |
| 206802_at | NM_016734 | PAX5 | 5079 | −0.09 | 3.66 | 3.369 | 3.254 | 3.512 | 3.547 | 3.376 | −0.134 | −0.055 |
| 206866_at | NM_001794 | CDH4 | 1002 | 1.385 | 2.88 | 3.045 | 3.169 | 3.175 | 3.193 | 3.072 | 0.209 | 0.169 |
| 206896_s_at | NM_005145 | GNG7 | 2788 | −0.03 | 4.02 | 4.328 | 4.079 | 4.181 | 4.478 | 4.64 | −0.045 | 0.384 |
| 206901_at | NM_024323 | C19orf57 | 79173 | −0.18 | 3.55 | 3.712 | 3.535 | 3.602 | 3.661 | 3.609 | −0.062 | 0.005 |
| 206923_at | NM_002737 | PRKCA | 5578 | −0.07 | 2.96 | 3.048 | 3.054 | 3.313 | 3.033 | 3.052 | 0.178 | 0.037 |
| 206951_at | NM_003545 | HIST1H4A /// | 121504 /// 554313 | −0.15 | 3.69 | 3.753 | 3.681 | 3.976 | 3.913 | 4.481 | 0.106 | 0.474 |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 206976_s_at | NM_006644 | HSPH1 | 10808 | 0.025 | 10.1 | 10.01 | 10.07 | 10.07 | 9.1 | 9.249 | 0.016 | −0.881 |
| 207040_s_at | NM_003932 | ST13 | 6767 | −0.6 | 9.28 | 9.265 | 9.282 | 9.208 | 9.161 | 9.154 | −0.0 | |
| 207046_at | NM_003548 | HIST1H4A /// | 121504 /// 554313 | 0.708 | 4.32 | 4.101 | 4.566 | 4.384 | 5.262 | 4.681 | 0.26 | |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 207127_s_at | NM_021644 | HNRNPH3 | 3189 | 0.337 | 7.05 | 7.295 | 7.369 | 7.264 | 7.173 | 7.133 | 0.14 | |
| 207188_at | NM_001258 | CDK3 | 1018 | −0.15 | 5.79 | 6.267 | 5.873 | 6.246 | 6.239 | 6.181 | 0.03 | |
| 207225_at | NM_001088 | AANAT | 15 | −0.22 | 2.66 | 2.658 | 2.599 | 2.613 | 2.539 | 2.692 | −0.05 | |
| 207243_x_at | NM_001743 | CALM1 /// CALM2 /// | 801 /// 805 /// 808 | −0.09 | 12.2 | 12.34 | 12.36 | 12.35 | 12.18 | 12.31 | 0.07 | |
| | | CALM3 | | | | | | | | | | |
| 207263_x_at | NM_017599 | VEZT | 55591 | 0.273 | 3.24 | 3.165 | 3.217 | 3.315 | 3.448 | 3.211 | 0.06 | |
| 207323_s_at | NM_002385 | MBP | 4155 | 0.022 | 3.04 | 3.014 | 2.917 | 2.836 | 2.9 | 3.209 | −0.15 | |
| 207342_at | NM_001297 | CNGB1 | 1258 | −0.18 | 3.06 | 2.95 | 2.982 | 2.775 | 2.886 | 2.948 | −0.12 | |
| 207358_x_at | NM_012090 | MACF1 | 23499 | −0.13 | 6.08 | 6.136 | 6.283 | 6.228 | 5.928 | 6.224 | 0.14 | |
| 207360_s_at | NM_002531 | NTSR1 | 4923 | 0.036 | 4.05 | 4.028 | 4.21 | 4.244 | 4.379 | 4.205 | 0.18 | |
| 207382_at | NM_003722 | TP63 | 8626 | 0.121 | 3.37 | 3.449 | 3.509 | 3.34 | 3.592 | 3.293 | 0.01 | |
| 207425_s_at | NM_006640 | 10-Sep | 10801 | −0.14 | 3.69 | 3.446 | 3.599 | 3.505 | 3.613 | 3.674 | −0.015 | 0.076 |
| 207434_s_at | NM_021603 | FXYD2 | 486 | 0.124 | 2.85 | 3.118 | 2.992 | 3.11 | 3.188 | 3.138 | 0.069 | 0.181 |
| 207442_at | NM_000759 | CSF3 | 1440 | −0.14 | 3.07 | 2.91 | 3.285 | 3.113 | 3.161 | 3.329 | 0.21 | 0.256 |
| 207453_s_at | NM_012266 | DNAJB5 | 25822 | −0.03 | 3.28 | 3.152 | 3.038 | 3.091 | 3 | 3.11 | −0.152 | −0.161 |
| 207518_at | NM_003647 | DGKE | 8526 | 0.047 | 3.43 | 3.253 | 3.486 | 3.28 | 3.107 | 3.68 | 0.042 | 0.053 |
| 207525_s_at | NM_005716 | GIPC1 | 10755 | 0.351 | 7.08 | 6.978 | 6.829 | 7.087 | 7.299 | 7.092 | −0.073 | 0.165 |
| 207535_s_at | NM_002502 | NFKB2 | 4791 | −0.05 | 4.41 | 4.697 | 4.314 | 4.612 | 4.62 | 4.364 | −0.089 | −0.06 |
| 207650_x_at | NM_000955 | PTGER1 | 5731 | −0.2 | 3.75 | 3.547 | 3.657 | 3.837 | 3.437 | 3.666 | 0.097 | −0.099 |
| 207661_s_at | NM_014631 | SH3PXD2A | 9644 | −0.01 | 3.11 | 3.136 | 2.999 | 3.315 | 3.108 | 3.025 | 0.032 | −0.058 |
| 207708_at | NM_021628 | ALOXE3 | 59344 | 0.244 | 3.28 | 3.242 | 3.725 | 3.597 | 3.624 | 3.557 | 0.398 | 0.328 |
| 207711_at | NM_015377 | C20orf117 | 140710 | −0.72 | 5.29 | 5.322 | 5.061 | 5.461 | 4.83 | 5.058 | −0.043 | −0.36 |
| 207712_at | NM_001187 | BAGE | 574 | 0.092 | 3.07 | 3.316 | 3.304 | 3.228 | 3.352 | 3.294 | 0.071 | 0.128 |
| 207714_s_at | NM_004353 | SERPINH1 | 871 | −0.61 | 7.54 | 7.625 | 7.65 | 7.554 | 6.388 | 6.135 | 0.018 | −1.323 |
| 207760_s_at | NM_006312 | NCOR2 | 9612 | 0.436 | 7.96 | 7.912 | 8.166 | 8.178 | 8.221 | 8.255 | 0.235 | 0.301 |
| 207821_s_at | NM_005607 | PTK2 | 5747 | −0.26 | 5.59 | 5.325 | 5.371 | 5.451 | 5.413 | 5.686 | −0.046 | 0.092 |
| 207832_at | NM_017451 | BAIAP2 | 10458 | 0.041 | 3.11 | 3.415 | 3.192 | 3.314 | 3.458 | 3.644 | −0.012 | 0.287 |
| 207838_x_at | NM_020524 | PBXIP1 | 57326 | −0 | 3.31 | 3.324 | 3.371 | 3.357 | 3.142 | 3.356 | 0.045 | −0.07 |
| 207921_x_at | NM_013952 | PAX8 | 7849 | −0.06 | 2.81 | 2.677 | 2.79 | 2.797 | 2.661 | 2.724 | 0.051 | −0.05 |
| 207923_x_at | NM_013953 | PAX8 | 7849 | −0.04 | 2.82 | 2.82 | 2.752 | 2.797 | 3.214 | 2.776 | −0.045 | 0.176 |
| 207924_x_at | NM_013992 | PAX8 | 7849 | 0.191 | 2.77 | 2.678 | 2.62 | 2.733 | 2.734 | 2.665 | −0.047 | −0.024 |
| 207929_at | NM_005314 | GRPR | 2925 | 0.043 | 3.36 | 3.348 | 3.615 | 3.28 | 3.447 | 3.376 | 0.094 | 0.058 |
| 208002_s_at | NM_007274 | ACOT7 | 11332 | 0.346 | 8.21 | 8.288 | 7.616 | 7.733 | 8.07 | 7.924 | −0.575 | −0.252 |
| 208003_s_at | NM_006599 | NFAT5 | 10725 | −0.13 | 6.69 | 6.364 | 6.349 | 6.233 | 6.283 | 6.197 | −0.234 | −0.285 |
| 208009_s_at | NM_014448 | ARHGEF16 | 27237 | −0.08 | 4.3 | 4.282 | 4.191 | 4.673 | 4.166 | 4.371 | 0.14 | −0.024 |
| 208018_s_at | NM_002110 | HCK | 3055 | 0.078 | 3.29 | 3.855 | 4.022 | 4.023 | 4.166 | 4.256 | 0.452 | 0.641 |
| 208026_at | NM_003540 | HIST1H4A /// | 121504 /// 554313 | −0.08 | 3.38 | 3.267 | 2.973 | 3.014 | 3.659 | 3.581 | −0.329 | 0.298 |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 208031_s_at | NM_000635 | RFX2 | 5990 | −0.24 | 3.08 | 3.126 | 3.049 | 3.314 | 3.11 | 3.032 | 0.07 [symbol] | |
| 208046_at | NM_003538 | HIST1H4A /// | 121504 /// 554313 | 0.034 | 3.5 | 3.283 | 3.272 | 3.056 | 3.416 | 3.552 | −0.22 [symbol] | |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 208076_at | NM_003539 | HIST1H4A /// | 121504 /// 554313 | −0.06 | 3.31 | 3.552 | 3.701 | 3.475 | 3.639 | 4.189 | 0.155 | 0.481 |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 208102_s_at | NM_002779 | PSD | 5662 | 0.094 | 3.1 | 2.954 | 2.957 | 3.21 | 3.291 | 3.136 | 0.056 | 0.186 |
| 208178_x_at | NM_007118 | TRIO | 7204 | 0.633 | 5.27 | 5.038 | 4.97 | 4.872 | 5.401 | 5.085 | −0.235 | 0.088 |
| 208180_s_at | NM_003543 | HIST1H4A /// | 121504 /// 554313 | 0.047 | 5.77 | 5.445 | 5.937 | 5.925 | 7.713 | 7.847 | 0.326 | 2.175 |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 208181_at | NM_003543 | HIST1H4A /// | 121504 /// 554313 | 0.247 | 2.74 | 2.665 | 2.962 | 2.854 | 3.527 | 3.85 | 0.207 | |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 208252_s_at | NM_004273 | CHST3 | 9469 | −0.04 | 3.22 | 2.898 | 2.968 | 2.937 | 2.947 | 2.717 | −0.107 | |
| 208272_at | NM_007321 | RANBP3 | 8498 | 0.021 | 3.46 | 3.305 | 3.103 | 3.443 | 3.369 | 3.514 | −0.108 | |
| 208315_x_at | NM_003300 | TRAF3 | 7187 | 0.364 | 3.7 | 3.945 | 3.825 | 3.909 | 3.996 | 4.136 | 0.047 | |
| 208333_at | NM_022363 | LHX5 | 64211 | 0.137 | 2.85 | 2.8 | 2.768 | 2.769 | 3.029 | 2.94 | −0.058 | |
| 208336_s_at | NM_004868 | GPSN2 | 9524 | −0.38 | 8.68 | 8.697 | 8.744 | 8.641 | 8.781 | 8.859 | 0.007 | |
| 208424_s_at | NM_020313 | CIAPIN1 | 57019 | 0.271 | 6.52 | 6.525 | 6.264 | 6.465 | 6.36 | 6.387 | −0.156 | |
| 208441_at | NM_015883 | IGF1R | 3480 | −0.12 | 3.13 | 2.918 | 3.089 | 2.88 | 3.01 | 2.909 | −0.04 | |
| 208580_x_at | NM_021968 | HIST1H4A /// | 121504 /// 554313 | 0.276 | 7.03 | 7.285 | 7.131 | 7.163 | 7.15 | 7.313 | −0.013 | |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 208589_at | NM_020389 | TRPC7 | 57113 | −0.11 | 2.37 | 2.421 | 2.428 | 2.555 | 2.581 | 2.875 | 0.096 | 0.332 |
| 208611_s_at | U83867 | SPTAN1 | 6709 | −0.05 | 4.55 | 4.833 | 4.938 | 5.088 | 5.138 | 5.337 | 0.324 | 0.548 |
| 208615_s_at | BF795101 | PTP4A2 | 8073 | −0.04 | 7.92 | 7.642 | 7.488 | 7.591 | 7.28 | 7.304 | −0.24 | −0.488 |
| 208616_s_at | U48297 | PTP4A2 | 8073 | 0.485 | 10.6 | 10.57 | 10.44 | 10.48 | 10.51 | 10.56 | −0.126 | −0.05 |
| 208617_s_at | AF208850 | PTP4A2 | 8073 | 0.059 | 8.34 | 8.152 | 8.21 | 8.055 | 7.6 | 7.605 | −0.112 | −0.643 |
| 208633_s_at | W61052 | MACF1 | 23499 | −0.05 | 4.74 | 4.897 | 4.786 | 4.896 | 4.747 | 4.8 | 0.021 | −0.047 |
| 208634_s_at | AB029290 | MACF1 | 23499 | 0.01 | 7.16 | 6.963 | 7.068 | 7.079 | 7.107 | 7.113 | 0.011 | 0.048 |
| 208657_s_at | AF142408 | 10-Sep | 10801 | −0.13 | 5.68 | 5.84 | 5.819 | 5.886 | 5.589 | 5.739 | 0.093 | −0.096 |
| 208666_s_at | BE866412 | ST13 | 6767 | −0.91 | 6.41 | 6.334 | 6.18 | 6.139 | 5.633 | 5.995 | −0.21 | −0.556 |
| 208667_s_at | U17714 | ST13 | 6767 | −0.81 | 8.04 | 7.879 | 7.869 | 7.979 | 7.793 | 7.907 | −0.036 | −0.11 |
| 208684_at | U24105 | COPA | 1314 | −0.3 | 8.24 | 8.198 | 8.203 | 8.128 | 8.192 | 8.169 | −0.052 | −0.037 |
| 208687_x_at | AF352832 | HSPA8 | 3312 | −0.49 | 11.7 | 11.54 | 11.6 | 11.62 | 11.07 | 11.1 | 0.007 | −0.517 |
| 208696_at | AF275798 | CCT5 | 22948 | 0.154 | 11.4 | 11.39 | 11.46 | 11.45 | 11.45 | 11.3 | 0.061 | −0.023 |
| 208713_at | BF724216 | HNRNPUL1 | 11100 | −0.15 | 7.09 | 7.318 | 7.411 | 7.286 | 7.368 | 7.017 | 0.144 | −0.011 |
| 208730_x_at | AA535244 | RAB2A | 5862 | −0.15 | 5.65 | 5.561 | 5.538 | 5.528 | 5.723 | 5.506 | −0.07 | 0.011 |
| 208731_at | AW158062 | RAB2A | 5862 | 0.069 | 8.65 | 8.722 | 8.836 | 8.686 | 8.646 | 8.614 | 0.073 | −0.058 |
| 208732_at | AI743756 | RAB2A | 5862 | 0.524 | 5.32 | 5.427 | 5.006 | 5.329 | 5.374 | 5.518 | −0.206 | 0.073 |
| 208733_at | AW301641 | RAB2A | 5862 | 0.39 | 2.83 | 2.677 | 2.624 | 2.389 | 2.526 | 2.609 | −0.248 | −0.187 |
| 208734_x_at | M28213 | RAB2A | 5862 | 0.313 | 8.87 | 8.897 | 8.856 | 8.869 | 8.993 | 8.9 | −0.022 | 0.062 |
| 208744_x_at | BG403660 | HSPH1 | 10808 | −0.32 | 9.79 | 9.741 | 9.627 | 9.87 | 8.772 | 8.804 | −0.017 | −0.978 |
| 208756_at | U36764 | EIF3I | 8668 | 0.014 | 9.19 | 9.306 | 9.193 | 9.185 | 9.473 | 9.482 | −0.061 | 0.227 |
| 208759_at | AF240468 | NCSTN | 23385 | −0.28 | 6 | 5.838 | 6.311 | 5.854 | 6.077 | 6.369 | 0.162 | 0.303 |
| 208760_at | AL031714 | UBE2I | 7329 | −0.54 | 6.93 | 6.839 | 6.925 | 7.134 | 6.957 | 6.928 | 0.146 | 0.059 |
| 208778_s_at | BC000665 | TCP1 | 6950 | 0.003 | 9.85 | 9.894 | 9.665 | 9.768 | 9.556 | 9.542 | −0.154 | −0.322 |
| 208781_x_at | AF062483 | SNX3 | 8724 | −0.28 | 9.34 | 9.419 | 9.422 | 9.317 | 9.32 | 9.382 | −0.008 | −0.027 |
| 208791_at | M25915 | CLU | 1191 | −0.62 | 6.84 | 7.106 | 7.029 | 7.134 | 7.076 | 7.177 | 0.11 | 0.155 |
| 208792_s_at | M25915 | CLU | 1191 | −0.78 | 7.76 | 7.447 | 7.643 | 7.728 | 7.739 | 7.744 | 0.083 | 0.139 |
| 208806_at | BE379542 | CHD3 | 1107 | −0.25 | 4.29 | 4.352 | 4.275 | 4.309 | 4.448 | 4.365 | −0.031 | 0.084 |
| 208807_s_at | U91543 | CHD3 | 1107 | 0.017 | 5.71 | 5.56 | 5.405 | 5.479 | 5.137 | 5.243 | −0.193 | −0.445 |
| 208810_at | AF080569 | DNAJB6 | 10049 | 0.141 | 8.68 | 8.861 | 8.919 | 9.065 | 8.526 | 8.316 | 0.223 | −0.348 |
| 208811_s_at | AF080569 | DNAJB6 | 10049 | 0.2 | 8.03 | 7.847 | 8.198 | 8.13 | 7.69 | 7.827 | 0.223 | −0.182 |
| 208813_at | BC000498 | GOT1 | 2805 | 0.797 | 8.62 | 8.698 | 8.575 | 8.685 | 8.902 | 8.831 | −0.03 | 0.206 |
| 208814_at | AA043348 | HSPA4 | 3308 | −0.49 | 5.83 | 5.987 | 5.853 | 5.777 | 5.62 | 5.682 | −0.092 | −0.257 |
| 208815_x_at | AB023420 | HSPA4 | 3308 | 0.352 | 10.2 | 10.15 | 10.01 | 10.03 | 10.1 | 10.02 | −0.131 | −9.09 |
| 208820_at | AL037339 | PTK2 | 5747 | 0.098 | 8.01 | 8.143 | 8.256 | 7.997 | 7.997 | 8.213 | 0.049 | 0.027 |
| 208837_at | BC000027 | TMED3 | 23423 | −0.08 | 9.19 | 8.998 | 9.005 | 8.996 | 9.215 | 9.219 | −0.093 | 0.123 |
| 208858_s_at | BC004998 | FAM62A | 23344 | 0.083 | 7.75 | 7.687 | 7.748 | 7.824 | 7.734 | 7.851 | 0.066 | 0.072 |
| 208874_x_at | BC002545 | PPP2R4 | 5524 | −0.03 | 6.76 | 7.137 | 7.299 | 7.069 | 7.16 | 7.206 | 0.235 | 0.234 |
| 208888_s_at | AI499095 | NCOR2 | 9612 | 0.098 | 3.02 | 3.062 | 2.962 | 3.117 | 3.084 | 3.019 | −0.001 | 0.01 |
| 208889_s_at | AI373205 | NCOR2 | 9612 | 0.157 | 3.59 | 3.64 | 3.381 | 3.486 | 3.461 | 3.45 | −0.18 | |
| 208929_x_at | BC004954 | RPL13 | 6137 | −0.09 | 12.5 | 12.46 | 12.51 | 12.42 | 12.33 | 12.4 | −0.005 | |
| 208968_s_at | BC002568 | CIAPIN1 | 57019 | 0.332 | 7.86 | 7.741 | 7.723 | 7.821 | 7.927 | 7.885 | −0.029 | |
| 208980_s_at | M26880 | RPS27A /// UBB /// | 6233 /// 7314 /// | −0.11 | 12.1 | 12.19 | 12.21 | 12.16 | 11.92 | 12.15 | 0.032 | |
| | | UBC | 7316 | | | | | | | | | |
| 208990_s_at | AF132362 | HNRNPH3 | 3189 | −0.49 | 9.55 | 9.415 | 9.479 | 9.474 | 8.713 | 8.737 | −0.008 | |
| 209010_s_at | AI797657 | TRIO | 7204 | 0.175 | 3 | 2.802 | 2.991 | 2.862 | 2.854 | 2.924 | 0.025 | |
| 209011_at | BF223718 | TRIO | 7204 | 0.235 | 4.83 | 4.704 | 4.706 | 4.676 | 4.754 | 4.561 | −0.077 | |
| 209012_at | AV718192 | TRIO | 7204 | 0.412 | 5.64 | 5.527 | 5.519 | 5.558 | 5.728 | 5.791 | −0.043 | |
| 209013_x_at | AF091395 | TRIO | 7204 | 0.625 | 5.3 | 4.93 | 5.02 | 4.802 | 5.097 | 5.164 | −0.202 | |
| 209015_s_at | BC002446 | DNAJB6 | 10049 | 0.113 | 6.72 | 7.034 | 6.841 | 6.791 | 7.193 | 7.043 | −0.06 | |
| 209029_at | AF193844 | COPS7A | 50813 | −0.37 | 6.6 | 6.57 | 6.667 | 6.944 | 5.593 | 5.611 | 0.219 | |
| 209036_s_at | BC001917 | MDH2 | 4191 | 0.189 | 11.2 | 11.22 | 11.17 | 11.17 | 11.1 | 11.01 | −0.036 | |
| 209050_s_at | AI421559 | RALGDS | 5900 | 0.299 | 6.73 | 6.818 | 6.597 | 6.649 | 7.331 | 7.276 | −0.153 | |
| 209051_s_at | AF295773 | RALGDS | 5900 | 0.086 | 4.38 | 4.366 | 4.46 | 4.626 | 4.498 | 4.737 | 0.17 | |
| 209072_at | M13577 | MBP | 4155 | 0.086 | 2.97 | 3.145 | 3.039 | 2.973 | 2.991 | 3.055 | −0.051 | |
| 209117_at | U79458 | WBP2 | 23558 | −0.01 | 5.37 | 5.508 | 5.382 | 5.408 | 5.655 | 5.556 | −0.045 | |
| 209130_at | BC003686 | SNAP23 | 8773 | −0.27 | 7.95 | 7.877 | 8.011 | 7.972 | 7.449 | 7.457 | 0.078 | |
| 209131_s_at | U55936 | SNAP23 | 8773 | −0.19 | 3.55 | 3.66 | 3.64 | 3.652 | 3.046 | 3.494 | 0.042 | |
| 209179_s_at | BC003164 | MBOAT7 | 79143 | 0.063 | 6.03 | 5.92 | 5.882 | 6.064 | 6.478 | 6.206 | −0.003 | |
| 209214_s_at | BC004817 | EWSR1 | 2130 | 0.25 | 7.81 | 7.829 | 7.84 | 7.858 | 7.768 | 7.914 | 0.028 | |
| 209216_at | BC000464 | WDR45 | 11152 | −0.09 | 6.82 | 6.812 | 6.883 | 6.859 | 7.277 | 7.203 | 0.054 | |
| 209217_s_at | BC000464 | WDR45 | 11152 | 0.011 | 5.88 | 5.812 | 5.993 | 6.085 | 6.345 | 6.206 | 0.195 | |
| 209229_s_at | BC002799 | SAPS1 | 22870 | 0.375 | 4.37 | 4.379 | 4.469 | 4.439 | 4.389 | 4.367 | 0.081 | |
| 209263_x_at | BC000389 | TSPAN4 | 7106 | −0.1 | 7.06 | 6.939 | 7.028 | 7.011 | 7.274 | 6.915 | 0.02 | |
| 209264_s_at | AF054841 | TSPAN4 | 7106 | −0.45 | 5.64 | 5.763 | 6.136 | 6.115 | 6.14 | 6.312 | 0.426 | |
| 209282_at | AF309082 | PRKD2 | 25865 | −0.24 | 4.19 | 4.209 | 4.197 | 4.211 | 4.221 | 4.132 | 0.003 | −0.024 |
| 209380_s_at | AF146074 | ABCCS | 10057 | −0.22 | 6.47 | 6.262 | 6.14 | 6.146 | 5.783 | 6.067 | −0.226 | −0.443 |
| 209388_at | BC000927 | PAPOLA | 10914 | 0.247 | 10.5 | 10.62 | 10.46 | 10.49 | 10.42 | 10.24 | −0.08 | −0.221 |
| 209428_s_at | BG420865 | ZFPL1 | 7542 | 0.161 | 6.09 | 6.084 | 6.227 | 6.297 | 6.176 | 6.474 | 0.177 | 0.24 |
| 209453_at | M81768 | SLC9A1 | 6548 | −0.05 | 4.19 | 4.268 | 4.443 | 4.272 | 4.137 | 4.019 | 0.129 | −0.15 |
| 209493_at | AF338650 | PDZD2 | 23037 | 0.015 | 3.4 | 3.58 | 3.409 | 3.645 | 3.727 | 3.688 | 0.339 | 0.219 |
| 209502_s_at | BC002495 | BAIAP2 | 10458 | 0.458 | 3.61 | 3.384 | 3.635 | 3.502 | 3.913 | 3.965 | 0.071 | 0.441 |
| 209516_at | U50383 | SMYD5 | 10322 | 0.047 | 4.65 | 4.254 | 4.559 | 4.386 | 4.725 | 4.577 | 0.02 | 0.198 |
| 209552_at | BC001060 | PAX8 | 7849 | 0.018 | 3 | 3.107 | 3.165 | 3.335 | 3.022 | 3.099 | 0.196 | 0.007 |
| 209563_x_at | BC000454 | CALM1 /// CALM2 /// | 801 /// 808 /// 808 | 0.047 | 10.9 | 10.85 | 10.8 | 10.81 | 10.83 | 11 | −0.088 | 0.022 |
| | | CALM3 | | | | | | | | | | |
| 209575_at | BC001903 | IL10RB | 3588 | −0.25 | 6.65 | 6.814 | 6.765 | 6.895 | 6.97 | 7.021 | 0.097 | 0.262 |
| 209579_s_at | AL556619 | MBD4 | 8930 | 0.205 | 10.3 | 10.25 | 10.14 | 10.2 | 10.49 | 10.54 | −0.092 | 0.251 |
| 209580_s_at | AF114784 | MBD4 | 8930 | 0.549 | 7.84 | 7.945 | 7.718 | 7.649 | 7.682 | 7.864 | −0.208 | −0.119 |
| 209590_at | AL57414 | BMP7 | 655 | −0.74 | 6.88 | 6.942 | 7.466 | 7.158 | 6.963 | 6.875 | 0.399 | −0.006 |
| 209591_s_at | M60316 | BMP7 | 655 | 0.042 | 9.89 | 10.02 | 10.22 | 10.26 | 10.25 | 10.26 | 0.284 | 0.3 |
| 209626_s_at | AL202969 | OSBPL3 | 26031 | 0.108 | 2.72 | 3.05 | 3.372 | 3.173 | 3.216 | 3.244 | 0.386 | 0.344 |
| 209627_s_at | AY008372 | OSBPL3 | 26031 | −0.24 | 2.66 | 3.117 | 3.368 | 2.996 | 3.049 | 3.033 | 0.293 | 0.152 |
| 209636_at | BC002844 | NFKB2 | 4791 | 0.131 | 2.7 | 2.764 | 2.692 | 2.584 | 2.929 | 2.719 | −0.094 | 0.092 |
| 209667_at | BF033242 | CES2 | 8824 | −0.13 | 6.37 | 6.314 | 6.165 | 6.398 | 6.748 | 6.632 | −0.06 | 0.349 |
| 209668_x_at | D50579 | CES2 | 8824 | −0.41 | 4.39 | 4.231 | 4.723 | 4.351 | 5.078 | 4.724 | 0.227 | 0.591 |
| 209674_at | D83702 | CRY1 | 1407 | 0.21 | 7.03 | 6.927 | 7.17 | 6.944 | 6.8 | 6.935 | 0.079 | −0.11 |
| 209675_s_at | BC004242 | HNRNPUL1 | 11100 | −0.32 | 6.01 | 6.323 | 6.282 | 6.276 | 5.934 | 5.936 | 0.114 | −0.23 |
| 209700_x_at | AB042555 | PDE4DIP | 9659 | 0.276 | 2.9 | 3.062 | 2.838 | 3.257 | 2.901 | 3.056 | 0.066 | −0.003 |
| 209736_at | AF116571 | SOX13 | 9580 | −0.09 | 5.33 | 5.698 | 5.646 | 5.585 | 5.525 | 5.365 | 0.103 | −0.068 |
| 209786_at | BC001282 | HMGN4 | 10473 | 0.156 | 8.1 | 8.018 | 8.031 | 7.948 | 7.858 | 7.808 | −0.071 | −0.227 |
| 209787_s_at | BC001282 | HMGN4 | 10473 | 0.226 | 9.22 | 9.243 | 9.273 | 9.226 | 9.184 | 9.089 | 0.019 | −0.094 |
| 209805_at | U14658 | PMS2 /// PMS2CL | 441194 /// 5395 | 0.335 | 6.22 | 6.47 | 6.271 | 6.413 | 6.389 | 6.178 | −0.005 | −0.064 |
| 209807_s_at | U18759 | NFIX | 4784 | −0.07 | 3.03 | 3.274 | 2.966 | 3.126 | 2.894 | 3.131 | −0.106 | −0.14 |
| 209820_s_at | BC002361 | TBL3 | 10607 | 0.201 | 5.41 | 5.378 | 5.641 | 5.377 | 5.478 | 5.511 | 0.115 | 0.1 |
| 209834_at | AB017915 | CHST3 | 9469 | 0.029 | 3.45 | 3.218 | 3.297 | 3.158 | 3.12 | 2.957 | −0.104 | −0.294 |
| 209849_s_at | AF029669 | RAD51C | 5889 | 0.052 | 10.4 | 10.36 | 10.52 | 10.43 | 9.93 | 10.04 | 0.11 | −0.377 |
| 209857_s_at | AF245447 | SPHK2 | 56848 | −0.07 | 3.09 | 3.467 | 3.353 | 3.363 | 3.206 | 3.286 | 0.08 | −0.032 |
| 209863_s_at | AF091627 | TP63 | 8626 | −0.18 | 3.69 | 3.161 | 3.84 | 3.846 | 3.993 | 4.102 | 0.418 | 0.623 |
| 209885_at | BC001338 | RHOD | 29984 | 0.378 | 7.71 | 7.478 | 7.693 | 7.788 | 7.891 | 7.931 | 0.147 | 0.317 |
| 209899_s_at | AF217197 | PUF60 | 22827 | 0.198 | 8.28 | 8.231 | 8.302 | 8.271 | 8.35 | 8.281 | 0.033 | 0.062 |
| 209934_s_at | AF225981 | ATP2C1 | 27032 | 0.598 | 5.21 | 5.446 | 5.12 | 5.247 | 5.331 | 5.369 | −0.146 | 0.02 |
| 209935_at | AF225981 | ATP2C1 | 27032 | 0.475 | 5.46 | 5.557 | 5.562 | 5.474 | 5.352 | 5.47 | 0.008 | −0.1 |
| 210011_s_at | BC000527 | EWSR1 | 2130 | 0.138 | 6.64 | 6.573 | 6.684 | 6.707 | 6.324 | 6.656 | 0.088 | −0. [symbol] |
| 210012_s_at | BC000527 | EWSR1 | 2130 | −0.27 | 3.59 | 3.62 | 3.619 | 3.533 | 3.518 | 3.207 | −0.028 | |
| 210043_at | AF334946 | FRMD8 | 83786 | 0.244 | 3.81 | 3.895 | 3.725 | 3.782 | 3.922 | 3.686 | −0.098 | |
| 210083_at | AF071542 | SEMA7A | 8482 | 0.117 | 3.27 | 3.395 | 3.21 | 3.224 | 3.423 | 3.438 | −0.114 | |
| 210110_x_at | AF132363 | HNRNPH3 | 3189 | 0.114 | 6.46 | 6.478 | 6.627 | 6.558 | 6.474 | 6.471 | 0.124 | |
| 210117_at | AF311312 | SPAG1 | 6674 | 0.536 | 6.08 | 6.052 | 5.969 | 5.916 | 5.878 | 5.982 | −0.122 | |
| 210120_s_at | BC004349 | RANBP3 | 8498 | 0.007 | 3.9 | 3.921 | 4.167 | 3.849 | 4.099 | 3.743 | 0.1 | |
| 210125_s_at | AF044773 | BANF1 | 8815 | −0.19 | 9.84 | 9.812 | 9.681 | 9.819 | 9.707 | 9.744 | −0.078 | |
| 210130_s_at | AF096304 | TM7SF2 | 7108 | −0.42 | 7.88 | 7.829 | 7.566 | 7.493 | 7.589 | 7.918 | −0.324 | |
| 210136_at | AW070431 | MBP | 4155 | 0.049 | 5.44 | 5.167 | 5.239 | 5.32 | 5.531 | 5.493 | −0.026 | |
| 210150_s_at | BC003355 | LAMA5 | 3911 | −0.25 | 6.03 | 5.755 | 5.749 | 5.864 | 5.839 | 5.884 | −0.085 | |
| 210180_s_at | U87836 | SFRS10 | 6434 | 0.18 | 8 | 8.131 | 7.783 | 7.979 | 7.567 | 7.71 | −0.184 | |
| 210211_s_at | AF028832 | HSP90AA1 | 3320 | −0.58 | 11.7 | 11.86 | 11.75 | 11.87 | 11.08 | 11.3 | 0.047 | |
| 210233_at | AF167343 | IL1RAP | 3556 | 0.572 | 5.17 | 5.144 | 5.507 | 5.515 | 5.544 | 5.006 | 0.353 | |
| 210255_at | U84138 | RAD51L1 | 5890 | −0.08 | 3.95 | 4.131 | 4.169 | 4.005 | 3.502 | 3.817 | 0.045 | |
| 210305_at | AB042557 | PDE4DIP | 9659 | 0.326 | 3.15 | 2.888 | 3.015 | 3.074 | 3.044 | 2.892 | 0.028 | |
| 210307_s_at | AL136796 | KLHL25 | 64410 | −0.16 | 5.58 | 5.618 | 5.328 | 5.28 | 5.563 | 5.606 | −0.296 | |
| 210331_at | AB048365 | HECW1 | 23072 | 0.193 | 3.08 | 3.14 | 2.951 | 2.997 | 2.976 | 2.854 | −0.134 | |
| 210338_s_st | AB034951 | HSPA8 | 3312 | −0.74 | 11.7 | 11.63 | 11.61 | 11.71 | 10.91 | 11.1 | −0.024 | |
| 210378_s_at | BC004118 | SSNA1 | 8636 | −0 | 7.44 | 7.289 | 7.252 | 7.258 | 7.1 | 7.332 | −0.108 | |
| 210407_at | AF070670 | PPM1A | 5494 | 0.262 | 7.06 | 7.135 | 6.954 | 7.018 | 7.075 | 6.846 | −0.11 | |
| 210426_x_at | U04897 | RORA | 6095 | −0.27 | 3.38 | 3.199 | 3.319 | 2.893 | 3.378 | 3.289 | −0.181 | |
| 210436_at | BC005220 | CCT8 | 10694 | −0.11 | 3.05 | 2.861 | 2.98 | 3.013 | 2.933 | 3.042 | 0.039 | |
| 210461_s_at | BC002448 | ABLIM1 | 3983 | 0.488 | 6.39 | 6.002 | 6.332 | 6.278 | 6.103 | 6.203 | 0.109 | |
| 210479_s_at | L14611 | RORA | 6095 | −0.12 | 3.43 | 3.321 | 3.101 | 3.141 | 3.409 | 3.409 | −0.252 | |
| 210550_s_at | L26584 | RASGRF1 | 5923 | −0.21 | 3.17 | 3.433 | 3.218 | 3.026 | 3.032 | 3.075 | −0.18 | |
| 210554_s_at | BC002486 | CTBP2 | 1488 | −0.24 | 9.23 | 9.295 | 9.125 | 9.178 | 9.033 | 8.887 | −0.109 | |
| 210574_s_at | AF241788 | NUDC | 10726 | 0.305 | 7.48 | 7.528 | 7.467 | 7.577 | 7.636 | 7.639 | 0.017 | 0.133 |
| 210575_at | AF241788 | NUDC | 10726 | −0.03 | 2.75 | 3.011 | 3.128 | 3.013 | 2.809 | 2.739 | 0.188 | −0.108 |
| 210588_x_at | L32610 | HNRNPH3 | 3189 | 0.298 | 7.68 | 8.098 | 8.189 | 8.092 | 7.925 | 8.079 | 0.252 | 0.113 |
| 210628_x_at | AF051344 | LTBP4 | 8425 | −0.2 | 3.46 | 3.42 | 3.699 | 3.502 | 3.422 | 3.501 | 0.162 | 0.022 |
| 210647_x_at | AF102988 | PLA2G6 | 8398 | −0.25 | 3.92 | 3.815 | 3.969 | 3.828 | 3.876 | 4.013 | 0.031 | 0.077 |
| 210648_x_at | AB047360 | SNX3 | 8724 | 0.05 | 10.8 | 10.67 | 10.73 | 10.7 | 10.97 | 10.9 | −0.015 | 0.204 |
| 210666_at | AF050145 | IDS | 3423 | 0.204 | 4.82 | 4.762 | 4.888 | 5.136 | 5.03 | 4.888 | 0.22 | 0.166 |
| 210691_s_at | AF275803 | CACYBP | 27101 | −0.46 | 9.54 | 9.559 | 9.578 | 9.592 | 8.802 | 8.82 | 0.037 | −0.737 |
| 210735_s_at | BC000278 | CA12 | 771 | −0.29 | 7.57 | 7.433 | 7.283 | 7.193 | 7.094 | 7.246 | −0.264 | −0.331 |
| 210752_s_at | AF213666 | MLX | 6945 | 0.272 | 3.43 | 3.45 | 3.707 | 3.511 | 3.703 | 3.878 | 0.171 | 0.353 |
| 210769_at | U18945 | CNGB1 | 1258 | 0.114 | 3.19 | 3.203 | 3.103 | 3.352 | 3.38 | 3.473 | 0.033 | 0.232 |
| 210780_at | AB006589 | ESR2 | 2100 | −0.14 | 3.15 | 3.12 | 3.081 | 3.052 | 3.113 | 3.156 | −0.068 | −4E−04 |
| 210821_x_at | BC002703 | CENPA | 1058 | −0.22 | 5.19 | 5.59 | 5.363 | 5.374 | 5.121 | 4.791 | −0.024 | −0.436 |
| 210835_s_at | AF222711 | CTBP2 | 1488 | −0.16 | 9.1 | 9.093 | 8.94 | 8.986 | 8.594 | 8.586 | −0.131 | −0.504 |
| 210878_s_at | BC001202 | JMJD1B | 51780 | 0.072 | 5.76 | 5.794 | 5.889 | 5.758 | 5.973 | 6.052 | 0.048 | 0.237 |
| 210933_s_at | BC004908 | FSCN1 | 6624 | 0.192 | 3.21 | 3.381 | 3.555 | 3.724 | 3.811 | 3.697 | 0.344 | 0.459 |
| 210956_at | U42387 | PPYR1 | 5540 | 0.084 | 3.06 | 2.911 | 2.955 | 3.26 | 2.865 | 3.06 | 0.121 | −0.023 |
| 210957_s_at | L76569 | AFF2 | 2334 | −0 | 2.86 | 2.711 | 2.859 | 2.939 | 2.793 | 2.84 | 0.114 | 0.032 |
| 210984_x_at | U95089 | EGFR | 1956 | −0.48 | 2.98 | 3.057 | 3.135 | 3.336 | 3.481 | 3.215 | 0.218 | 0.33 |
| 211004_s_at | BC002553 | ALDH3B1 | 221 | −0.27 | 4.9 | 4.884 | 4.939 | 4.892 | 4.828 | 4.404 | 0.022 | −0.278 |
| 211008_s_at | BC000744 | UBE2I | 7329 | −0.15 | 3.14 | 3.008 | 2.962 | 3.111 | 3.034 | 3.256 | −0.037 | 0.072 |
| 211015_s_at | L12723 | HSPA4 | 3308 | 0.157 | 9.71 | 9.872 | 9.666 | 9.703 | 9.633 | 9.588 | −0.106 | −0.18 |
| 211016_x_at | BC002526 | HSPA4 | 3308 | −0.02 | 8.35 | 8.219 | 8.149 | 8.054 | 7.938 | 8.019 | −0.185 | −0.308 |
| 211028_s_at | BC006233 | KHK | 3795 | −0.21 | 3.75 | 3.83 | 4.046 | 4.24 | 3.579 | 3.818 | 0.351 | −0.094 |
| 211037_s_at | BC006309 | MBOAT7 | 79143 | −0.02 | 4.27 | 4.476 | 4.302 | 4.294 | 3.726 | 3.998 | −0.077 | −0.514 |
| 211078_s_at | Z25422 | STK3 | 6788 | 0.314 | 4.64 | 4.831 | 4.906 | 5.084 | 5.038 | 4.877 | 0.261 | 0.224 |
| 211085_s_at | Z25430 | STK4 | 6789 | −0.03 | 6.14 | 6.411 | 6.381 | 6.514 | 6.641 | 6.464 | 0.171 | 0.276 |
| 211093_at | U31973 | PDE6C | 5146 | −0.05 | 2.51 | 2.495 | 2.575 | 2.588 | 2.625 | 2.489 | 0.079 | 0.055 |
| 211099_s_at | U58837 | CNGB1 | 1258 | 0.104 | 2.8 | 3.012 | 2.861 | 2.934 | 3.067 | 3.033 | −0.007 | 0.146 |
| 211117_x_at | AF124790 | ESR2 | 2100 | −0.05 | 2.73 | 2.691 | 2.831 | 2.882 | 2.741 | 2.777 | 0.146 | 0.048 |
| 211118_x_at | AF051428 | ESR2 | 2100 | −0.22 | 3.14 | 2.798 | 2.863 | 2.868 | 2.787 | 2.927 | −0.103 | −0.112 |
| 211119_at | AF060555 | ESR2 | 2100 | −0.05 | 2.65 | 2.609 | 2.583 | 2.451 | 2.577 | 2.573 | −0.113 | −0.055 |
| 211120_x_at | AB006590 | ESR2 | 2100 | −0.16 | 2.63 | 2.815 | 2.503 | 2.58 | 2.647 | 2.622 | −0.182 | −0.089 |
| 211137_s_at | AF189723 | ATP2C1 | 27032 | 0.502 | 5.99 | 5.905 | 5.685 | 5.542 | 5.977 | 5.92 | −0.332 | 0.003 |
| 211194_s_at | AB010153 | TP63 | 8626 | 0.437 | 2.95 | 2.778 | 3.047 | 3.162 | 3.295 | 3.106 | 0.239 | 0.335 |
| 211195_s_at | AF116771 | TP63 | 8626 | −0.2 | 3.16 | 3.131 | 3.231 | 3.036 | 2.903 | 2.949 | −0.013 | −0.22 |
| 211200_s_at | BC002836 | EFCAB2 | 84288 | 0.433 | 5.99 | 5.887 | 5.866 | 5.837 | 5.576 | 5.544 | −0.085 | −0.376 |
| 211225_at | U27329 | FUT5 | 2527 | −0.28 | 3.61 | 3.308 | 3.47 | 3.772 | 3.743 | 3.878 | 0.16 | 0.35 |
| 211259_s_at | BC004248 | BMP7 | 655 | −0.05 | 7.33 | 7.48 | 7.768 | 7.784 | 7.74 | 7.711 | 0.373 | 0.323 |
| 211260_at | BC004248 | BMP7 | 655 | −0.24 | 7.04 | 7.226 | 7.206 | 7.307 | 7.134 | 6.985 | 0.126 | |
| 211266_s_at | U35399 | GPR4 | 2828 | −0.01 | 2.84 | 2.874 | 2.787 | 2.827 | 3.169 | 2.763 | −0.052 | |
| 211277_x_at | BC004369 | APP | 351 | −0.27 | 5.97 | 6.005 | 5.673 | 6.198 | 5.696 | 5.85 | −0.053 | |
| 211296_x_at | AB009010 | RPS27A /// UBB /// | 6233 /// 7314 /// | −0.09 | 13 | 12.95 | 13.01 | 12.96 | 12.99 | 12.99 | 0.005 | |
| | | UBC | 7316 | | | | | | | | | |
| 211323_s_at | L38019 | ITPR1 | 3708 | −0.03 | 3.3 | 3.213 | 3.645 | 3.422 | 3.316 | 3.448 | 0.276 | |
| 211345_x_at | AF119850 | EEF1G | 1937 | −0.14 | 12.3 | 12.21 | 12.29 | 12.2 | 12.29 | 12.27 | −0.024 | |
| 211426_x_at | U40038 | GNAQ | 2776 | −0.43 | 4.51 | 4.279 | 4.588 | 4.428 | 4.082 | 4.018 | 0.115 | |
| 211428_at | AF119873 | SERPINA1 | 5265 | 0.018 | 3.01 | 2.763 | 2.929 | 2.926 | 2.911 | 2.915 | 0.042 | |
| 211429_s_at | AF119873 | SERPINA1 | 5265 | −0.55 | 6.08 | 6.698 | 6.074 | 6.164 | 6.35 | 6.115 | −0.268 | |
| 211439_at | AF055270 | SFRS7 | 6432 | 0.022 | 3.72 | 3.35 | 3.706 | 3.493 | 3.217 | 3.442 | 0.062 | |
| 211524_at | U09609 | NFKB2 | 4791 | −0.03 | 3.1 | 3.158 | 3.005 | 2.881 | 2.94 | 2.908 | −0.187 | |
| 211550_at | AF125253 | EGFR | 1956 | −0.15 | 3.03 | 3.012 | 3.004 | 2.896 | 3.131 | 2.91 | −0.071 | |
| 211551_at | K03193 | EGFR | 1956 | 0.11 | 3.62 | 3.59 | 3.395 | 3.5 | 3.812 | 3.703 | −0.15 [symbol] | |
| 211579_at | U95204 | ITGB3 | 3690 | −0.11 | 2.89 | 2.866 | 2.641 | 2.858 | 2.796 | 3.006 | −0.1 [symbol] | |
| 211607_x_at | U48722 | EGFR | 1956 | −0.61 | 3.49 | 3.108 | 3.036 | 3.164 | 3.379 | 3.177 | −0.19 [symbol] | |
| 211685_s_at | AF251061 | NCALD | 83988 | 0.027 | 3.75 | 3.317 | 3.742 | 3.331 | 3.627 | 3.449 | 0.004 | |
| 211711_s_at | BC005821 | PTEN | 5728 | −0.45 | 5.79 | 5.823 | 5.911 | 5.971 | 6.426 | 6.446 | 0.136 | |
| 211730_s_at | BC005903 | POLR2L | 5441 | 0.047 | 9.42 | 9.476 | 9.275 | 9.463 | 9.844 | 9.809 | −0.0 [symbol] | |
| 211751_at | BC005949 | PDE4DIP | 9659 | 0.071 | 3.55 | 3.821 | 3.591 | 3.196 | 3.609 | 3.497 | −0.29 [symbol] | |
| 211761_s_at | BC005975 | CACYBP | 27101 | −0.4 | 9.58 | 9.59 | 9.615 | 9.607 | 9.324 | 9.382 | 0.02 [symbol] | |
| 211763_s_at | BC005979 | UBE2B | 7320 | −0.07 | 7.2 | 7.108 | 7.063 | 7.178 | 7.28 | 7.299 | −0.03 [symbol] | |
| 211782_at | BC006170 | IDS | 3423 | −0.01 | 2.96 | 3.09 | 3.386 | 3.056 | 3.232 | 3.035 | 0.19 [symbol] | |
| 211790_s_at | AF010404 | MLL2 | 8085 | 0.014 | 2.76 | 2.645 | 2.827 | 2.639 | 3.001 | 2.706 | 0.031 | |
| 211828_s_at | AF172268 | TNIK | 23043 | −0.32 | 3.87 | 4.601 | 4.515 | 4.119 | 4.334 | 3.825 | 0.081 | |
| 211834_s_at | AB042841 | TP63 | 8626 | −0.04 | 3.11 | 3.185 | 3.195 | 2.957 | 3.112 | 3.116 | −0.0 [symbol] | |
| 211907_s_at | AB044555 | PARD6B | 84612 | −0.09 | 4.26 | 4.374 | 4.362 | 4.592 | 5.021 | 5.142 | 0.161 | 0.765 |
| 211927_x_at | BE963164 | EEF1G | 1937 | −0.08 | 12.7 | 12.64 | 12.7 | 12.64 | 12.61 | 12.57 | 0.007 | −0.074 |
| 211943_x_at | AL565449 | TPT1 | 7178 | −0.03 | 12.9 | 12.94 | 12.97 | 12.89 | 12.82 | 12.81 | −0.002 | −0.117 |
| 211968_s_at | AI962933 | HSP90AA1 | 3320 | −0.5 | 11.9 | 11.86 | 11.86 | 11.87 | 11.5 | 11.46 | 0.004 | −0.388 |
| 211969_at | BG420237 | HSP90AA1 | 3320 | −0.4 | 12.5 | 12.52 | 12.47 | 12.43 | 12.21 | 12.19 | −0.056 | −0.313 |
| 211984_at | AI653730 | CALM1 /// CALM2 /// | 801 /// 805 /// 808 | 0.472 | 9.25 | 9.065 | 9.203 | 9.247 | 9.536 | 9.531 | 0.068 | 0.377 |
| | | CALM3 | | | | | | | | | | |
| 211985_s_at | AI653730 | CALM1 /// CALM2 /// | 801 /// 805 /// 808 | −0.04 | 7.87 | 7.553 | 7.807 | 7.728 | 7.551 | 7.944 | 0.008 | −0.012 |
| | | CALM3 | | | | | | | | | | |
| 212009_s_at | AL553320 | STIP1 | 10963 | −0.17 | 9.49 | 9.464 | 9.366 | 9.42 | 9.106 | 9.175 | −0.083 | −0.336 |
| 212012_at | BF342851 | PXDN | 7837 | −0.79 | 8.15 | 8.088 | 8.025 | 7.837 | 8.028 | 8.087 | −0.188 | −0.061 |
| 212013_at | D86983 | PXDN | 7837 | −0.56 | 6.47 | 6.611 | 6.439 | 6.488 | 6.549 | 6.339 | −0.077 | −0.097 |
| 212027_at | AI925305 | RBM25 | 58517 | 0.313 | 8.54 | 8.705 | 8.564 | 8.654 | 8.499 | 8.524 | −0.016 | −0.113 |
| 212028_at | BE466128 | RBM25 | 58517 | 0.385 | 8.4 | 8.28 | 8.234 | 8.338 | 8.212 | 8.346 | −0.052 | −0.058 |
| 212030_at | BG251218 | RBM25 | 58517 | 0.336 | 7 | 7.091 | 7.231 | 7.302 | 7.163 | 7.268 | 0.221 | 0.171 |
| 212031_at | AV757384 | RBM25 | 58517 | 0.288 | 7.77 | 7.683 | 7.775 | 7.734 | 7.853 | 7.827 | 0.028 | 0.113 |
| 212032_s_at | AL046054 | PTOV1 | 53635 | −0.45 | 6.3 | 6.323 | 6.331 | 6.046 | 6.478 | 6.341 | −0.122 | 0.099 |
| 212033_at | BF055107 | RBM25 | 58517 | 0.293 | 8.32 | 8.24 | 8.19 | 8.281 | 8.263 | 8.294 | −0.043 | −2E−04 |
| 212070_at | AL554008 | GPRS6 | 9289 | 0.343 | 7.69 | 7.456 | 7.648 | 7.568 | 7.825 | 7.683 | 0.037 | 0.183 |
| 212076_at | AI701430 | MLL | 4297 | −0.18 | 5.81 | 5.831 | 5.879 | 5.874 | 5.811 | 5.495 | 0.055 | −0.169 |
| 212078_s_at | AA704766 | MLL | 4297 | −0.07 | 5.91 | 5.797 | 6.033 | 6.253 | 5.738 | 5.97 | 0.29 | 9E−04 |
| 212079_s_at | AA715041 | MLL | 4297 | −0.32 | 6.22 | 6.182 | 6.178 | 6.468 | 5.958 | 6.252 | 0.121 | −0.097 |
| 212080_at | AV714029 | MLL | 4297 | 0.052 | 5.85 | 5.805 | 5.826 | 5.741 | 5.843 | 5.768 | −0.045 | −0.024 |
| 212082_s_at | BE734356 | MYL6 /// MYL6B | 140465 /// 4637 | −0.13 | 12.1 | 12.21 | 12.16 | 12.05 | 11.87 | 12.09 | −0.071 | −0.197 |
| 212088_at | BF570122 | PMPCA | 23203 | 0.274 | 8.3 | 8.446 | 8.5 | 8.447 | 8.907 | 8.841 | 0.098 | 0.498 |
| 212125_at | NM_002883 | RANGAP1 | 5905 | −0.4 | 6.36 | 6.528 | 6.436 | 6.478 | 6.261 | 6.371 | 0.013 | −0.129 |
| 212127_at | BE379408 | RANGAP1 | 5905 | 0.1 | 5.42 | 5.566 | 5.542 | 5.789 | 5.613 | 5.606 | 0.173 | 0.118 |
| 212191_x_at | AW574664 | RPL13 | 6137 | −0.07 | 12.8 | 12.72 | 12.7 | 12.74 | 12.74 | 12.77 | −0.016 | 0.018 |
| 212194_s_at | AI418892 | TM9SF4 | 9777 | 0.093 | 5.69 | 5.837 | 5.662 | 5.867 | 5.879 | 5.748 | 0.003 | 0.053 |
| 212198_s_at | AL515964 | TM9SF4 | 9777 | −0.22 | 4.4 | 4.64 | 4.898 | 4.812 | 4.955 | 5.032 | 0.337 | 0.476 |
| 212221_x_at | AV703259 | IDS | 3423 | 0.506 | 7.91 | 7.761 | 8.062 | 7.899 | 7.939 | 7.93 | 0.144 | 6.097 |
| 212223_at | AI926544 | IDS | 3423 | 0.026 | 6.24 | 6.263 | 6.303 | 6.212 | 6.192 | 6.35 | 0.008 | 0.021 |
| 212228_s_at | AC004382 | COQ9 | 57017 | 0.153 | 7.25 | 7.315 | 7.347 | 7.475 | 7.814 | 7.688 | 0.128 | 0.468 |
| 212255_s_at | AK001684 | ATP2C1 | 27032 | 0.259 | 6.67 | 6.582 | 6.62 | 6.618 | 6.727 | 6.646 | −0.007 | 0.06 |
| 212259_s_at | BF344265 | PBXIP1 | 57326 | −0.36 | 4.31 | 4.315 | 3.931 | 3.842 | 4.093 | 4.162 | −0.425 | −0.184 |
| 212284_x_at | BG498776 | TPT1 | 7178 | −0.05 | 13.2 | 13.14 | 13.17 | 13.09 | 13.07 | 13.08 | −0.022 | −0.074 |
| 212317_at | AK022910 | TNPO3 | 23534 | 0.063 | 7.9 | 7.736 | 7.81 | 7.653 | 7.83 | 7.81 | −0.085 | 0.003 |
| 212318_at | NM_012470 | TNPO3 | 23534 | −0.02 | 7.9 | 7.858 | 8.071 | 8.01 | 7.895 | 7.814 | 0.164 | −0.022 |
| 212338_at | AA621962 | MYO1D | 4642 | 0.383 | 4.43 | 4.601 | 4.789 | 4.614 | 4.398 | 4.692 | 0.187 | 0.031 |
| 212348_s_at | AB011173 | AOF2 | 23028 | −0.13 | 6.62 | 6.928 | 6.808 | 6.849 | 6.67 | 6.709 | 0.054 | |
| 212367_at | AI799061 | FEM1B | 10116 | 0.319 | 7.51 | 7.29 | 7.43 | 7.346 | 7.58 | 7.901 | −0.013 | |
| 212373_at | AW139179 | FEM1B | 10116 | 0.575 | 5.56 | 5.478 | 5.418 | 5.453 | 5.828 | 5.879 | −0.085 | |
| 212374_at | NM_015322 | FEM1B | 10116 | 0.613 | 4.63 | 4.412 | 4.604 | 4.621 | 4.639 | 4.602 | 0.091 | |
| 212394_at | D42044 | KIAA0090 | 23065 | −0.21 | 3.28 | 3.464 | 3.269 | 3.394 | 3.901 | 3.899 | −0.039 | |
| 212395_s_at | BF197122 | KIAA0090 | 23065 | 0.129 | 5.09 | 5.176 | 5.207 | 5.383 | 5.304 | 5.27 | 0.164 | |
| 212396_s_at | AI143233 | KIAA0090 | 23065 | 0.182 | 6.02 | 5.914 | 5.712 | 5.86 | 6.256 | 6.134 | −0.181 | |
| 212411_at | BE747342 | IMP4 | 92856 | 0.117 | 9.29 | 9.318 | 9.2 | 9.402 | 9.491 | 9.486 | −0.003 | |
| 212421_at | AB023147 | C22orf9 | 23313 | −0.29 | 4.37 | 3.848 | 3.839 | 4.008 | 4.159 | 4.522 | −0.188 | |
| 212422_at | AL547263 | PDCD11 | 22984 | 0.728 | 6.01 | 6.08 | 5.886 | 5.994 | 6.004 | 6.112 | −0.106 | |
| 212424_at | AW026194 | PDCD11 | 22984 | 0.822 | 5.48 | 5.5 | 5.636 | 5.623 | 5.683 | 5.673 | 0.141 | |
| 212433_x_at | AA630314 | RPS2 | 6187 | −0.12 | 13.1 | 13.07 | 13.08 | 13.09 | 12.94 | 13.02 | 0.028 | |
| 212445_s_at | AI357376 | NEDD4L | 23327 | −0.19 | 4.49 | 4.303 | 4.176 | 4.237 | 4.294 | 4.309 | −0.189 | |
| 212448_at | AB007899 | NEDD4L | 23327 | 0.234 | 3.75 | 3.549 | 3.743 | 4.051 | 3.63 | 3.715 | 0.248 | |
| 212458_at | H97931 | SPRED2 | 200734 | 0.041 | 6.6 | 6.479 | 6.737 | 6.747 | 6.522 | 6.645 | 0.202 | |
| 212461_at | BF793951 | AZIN1 | 51582 | 0.365 | 9.06 | 9.031 | 9.009 | 8.982 | 8.864 | 8.886 | −0.048 | |
| 212463_at | BE379006 | CD59 | 966 | −0.03 | 5.55 | 5.594 | 5.606 | 5.508 | 5.742 | 5.641 | −0.014 | |
| 212466_at | AW138902 | SPRED2 | 200734 | −0.02 | 3.2 | 2.99 | 3.078 | 3.028 | 3.226 | 3.281 | −0.042 | |
| 212472_at | BE965029 | MICAL2 | 9645 | 1.595 | 3.94 | 3.664 | 4.101 | 3.942 | 4.552 | 4.163 | 0.219 | |
| 212473_s_at | BE965029 | MICAL2 | 9645 | 1.526 | 5.49 | 5.552 | 5.758 | 5.584 | 6.551 | 6.337 | 0.149 | |
| 212523_s_at | D63480 | KIAA0146 | 23514 | −0.5 | 3.83 | 4.281 | 4.295 | 4.247 | 3.716 | 4.048 | 0.217 | |
| 212551_at | NM_006366 | CAP2 | 10486 | 0.063 | 8.8 | 8.712 | 8.693 | 8.783 | 8.932 | 9.046 | −0.016 | |
| 212554_at | N90755 | CAP2 | 10486 | 0.064 | 8.84 | 8.746 | 8.917 | 8.738 | 9.005 | 8.881 | 0.036 | |
| 212574_x_at | AC004528 | C190rf6 | 91304 | −0.43 | 3.11 | 3.139 | 3.039 | 3.353 | 3.21 | 3.013 | 0.073 | |
| 212575_at | BF966155 | C19orf6 | 91304 | −0.01 | 3.98 | 4.071 | 3.905 | 4.06 | 3.762 | 3.901 | −0.045 | |
| 212611_at | AV728526 | DTX4 | 23220 | −0.63 | 5.1 | 5.331 | 5.12 | 5.104 | 5.231 | 5.35 | −0.106 | |
| 212647_at | NM_006270 | RRAS | 6237 | 0.049 | 5.35 | 5.674 | 5.625 | 5.809 | 5.773 | 5.591 | 0.205 | 0.17 |
| 212718_at | BF797555 | PAPOLA | 10914 | 0.185 | 10.2 | 10.01 | 10.03 | 10.03 | 10.36 | 10.3 | −0.086 | 0.206 |
| 212720_at | A1670847 | PAPOLA | 10914 | 0.007 | 6.85 | 6.679 | 6.597 | 6.741 | 6.349 | 6.513 | −0.093 | −0.334 |
| 212722_s_at | AK021780 | JMJD6 | 23210 | −0.07 | 5.05 | 5.054 | 5.155 | 5.241 | 4.749 | 4.834 | 0.146 | −0.261 |
| 212723_at | 4K021780 | LMLD6 | 23210 | 0.279 | 7.47 | 7.232 | 7.186 | 7.365 | 7.201 | 7.31 | −0.074 | −0.094 |
| 212734_x_at | AI186735 | RPL13 | 6137 | −0.56 | 13.1 | 13.13 | 13.09 | 13.08 | 13.08 | 13.08 | −0.032 | −0.037 |
| 212777_at | L13857 | SOS1 | 6654 | 0.413 | 4.24 | 3.837 | 4.041 | 4.139 | 4.167 | 3.976 | 0.054 | 0.035 |
| 212780_at | AA700167 | SOS1 | 6654 | 0.484 | 6.5 | 6.146 | 6.103 | 6.13 | 6.883 | 6.802 | −0.204 | 0.521 |
| 212816_s_at | BE613178 | CBS | 875 | 0.531 | 5.58 | 5.724 | 5.76 | 5.891 | 6.203 | 6.152 | 0.175 | 0.527 |
| 212817_at | AK023253 | DNAJB5 | 25822 | 0.217 | 3.94 | 3.725 | 3.635 | 3.76 | 3.906 | 3.92 | −0.135 | 0.08 |
| 212848_s_at | BG036668 | C9orf3 | 84909 | −0.14 | 7.73 | 7.508 | 7.426 | 7.578 | 8.52 | 8.702 | −0.118 | 0.991 |
| 212858_at | AL520675 | PAQR4 | 124222 | 0.452 | 5.02 | 5.333 | 5.47 | 5.54 | 5.063 | 5.072 | 0.329 | −0.108 |
| 212869_x_at | AI721229 | TPT1 | 7178 | −0.09 | 13.1 | 13.07 | 13.13 | 13.06 | 13.03 | 13.07 | −0.004 | −0.047 |
| 212873_at | BE349017 | HMHA1 | 23526 | 0.213 | 4.47 | 4.643 | 4.858 | 4.763 | 4.944 | 4.844 | 0.256 | 0.34 |
| 212877_at | AA284075 | KLC1 | 3831 | 0.326 | 7.37 | 7.502 | 7.471 | 7.177 | 7.958 | 8.043 | −0.112 | 0.565 |
| 212878_s_at | AA284075 | KLC1 | 3831 | 0.538 | 8.22 | 8.331 | 8.282 | 8.248 | 8.736 | 8.738 | −0.012 | 0.46 |
| 212898_at | AB007866 | KIAA0406 | 9675 | −0.38 | 7.33 | 7.509 | 7.33 | 7.328 | 6.561 | 6.319 | −0.09 | −0.978 |
| 212910_at | W19873 | THAP11 | 57215 | −0.13 | 7.63 | 7.568 | 7.612 | 7.591 | 7.668 | 7.775 | 0.004 | 0.125 |
| 212924_s_at | N37057 | LSM4 | 25804 | 0.213 | 4.72 | 5.059 | 4.82 | 4.801 | 5.097 | 4.907 | −0.081 | 0.111 |
| 212933_x_at | AA961748 | RPL13 | 6137 | −0.13 | 12.1 | 12.04 | 12.09 | 12.05 | 11.9 | 11.98 | −0.02 | −0.152 |
| 212944_at | AK024896 | SLCSA3 | 6526 | −0.54 | 6.57 | 6.444 | 6.12 | 6.335 | 6.187 | 6.092 | −0.28 | −0.368 |
| 212970_at | AI694303 | APBB2 | 323 | 0.725 | 6.14 | 6.046 | 5.809 | 5.77 | 6.429 | 6.347 | −0.301 | 0.297 |
| 212971_at | AI769685 | CARS | 833 | 0.43 | 9.24 | 9.295 | 9.466 | 9.36 | 9.968 | 9.797 | 0.145 | 0.614 |
| 212972_x_at | AL080130 | APBB2 | 323 | 0.068 | 4.31 | 4.381 | 4.107 | 4.359 | 4.401 | 4.477 | −0.115 | 0.091 |
| 212974_at | AI808958 | DENND3 | 22898 | −0.25 | 2.98 | 3.105 | 2.982 | 3.005 | 2.918 | 2.815 | −0.046 | −0.173 |
| 212975_at | AB020677 | DENND3 | 22898 | −0.07 | 3.73 | 3.874 | 3.821 | 3.375 | 3.71 | 3.626 | −0.207 | −0.136 |
| 212985_at | BF115739 | APBB2 | 323 | 0.701 | 6.86 | 6.913 | 6.719 | 6.641 | 7.261 | 7.226 | −0.207 | 0.357 |
| 212992_at | AI935123 | AHNAK2 | 113146 | 0.073 | 4.48 | 4.29 | 4.208 | 4.642 | 5.239 | 5.047 | 0.041 | 0.759 |
| 213010_at | AI088622 | PRKCDBP | 112464 | 0.223 | 3.99 | 4.202 | 3.823 | 3.954 | 4.376 | 4.162 | −0.206 | 0.175 |
| 213017_at | AL534702 | ABHD3 | 171586 | −0.24 | 7.16 | 7.087 | 7.157 | 7.148 | 7.028 | 6.96 | 0.027 | −0.132 |
| 213043_s_at | AI023317 | MED24 | 9862 | −0.23 | 4.65 | 4.842 | 4.593 | 5.063 | 4.808 | 4.833 | 0.082 | 0.075 |
| 213072_at | AI928387 | CYHR1 | 50625 | 0.002 | 3.88 | 3.546 | 3.501 | 3.63 | 4.006 | 3.999 | −0.147 | 0.289 |
| 213076_at | D38169 | ITPKC | 80271 | −0.06 | 4.43 | 4.352 | 4.595 | 4.389 | 4.818 | 4.863 | 0.103 | 0.451 |
| 213087_s_at | BF690020 | EEF1D | 1936 | 0.592 | 5.45 | 5.06 | 5.458 | 5.419 | 5.987 | 5.472 | 0.182 | 0.473 |
| 213093_at | AI471375 | PRKCA | 5578 | 0.273 | 3.33 | 3.54 | 3.364 | 3.376 | 3.536 | 3.664 | −0.065 | 0.165 |
| 213099_at | AB018302 | ANGEL1 | 23357 | −0.01 | 5.72 | 5.763 | 5.952 | 5.976 | 6.388 | 6.202 | 0.222 | 0.553 |
| 213107_at | R59093 | TNIK | 23043 | −0.15 | 5.15 | 5.196 | 5.311 | 5.407 | 4.926 | 5.14 | 0.184 | −0.142 |
| 213109_at | N25621 | TNIK | 23043 | −0.36 | 4.61 | 4.736 | 4.778 | 4.951 | 4.354 | 4.131 | 0.19 | −0.433 |
| 213124_at | BG538800 | ZNF473 | 25888 | 0.329 | 4.43 | 4.3 | 4.676 | 4.551 | 4.495 | 4.319 | 0.248 | 0.041 |
| 213130_at | AB032967 | 2NF473 | 25888 | 0.309 | 4.27 | 4.141 | 4.017 | 4.231 | 4.174 | 4.374 | −0.08 | |
| 213164_at | AI867198 | SLC5A3 | 6526 | −0.67 | 6.49 | 6.304 | 6.173 | 6.132 | 5.849 | 6.07 | −0.244 | |
| 213167_s_at | BF982927 | SLC5A3 | 6526 | −0.34 | 2.83 | 2.725 | 2.916 | 2.826 | 2.569 | 2.751 | 0.094 | |
| 213176_s_at | AI910869 | LTBP4 | 8425 | −0.61 | 3.77 | 3.814 | 4.297 | 3.754 | 3.685 | 3.676 | 0.233 | |
| 213252_at | AI739005 | SH3PXD2A | 9644 | 0.071 | 3.48 | 3.652 | 3.155 | 3.164 | 3.673 | 3.332 | −0.408 | |
| 213268_at | Z98884 | CAMTA1 | 23261 | 0.245 | 3.29 | 3.477 | 3.849 | 3.462 | 4.281 | 4.074 | 0.27 | |
| 213288_at | AI761250 | MBOAT2 | 129642 | −0.09 | 5.23 | 5.44S | 5.439 | 5.588 | 5.115 | 5.259 | 0.176 | |
| 213302_at | AL044326 | PFAS | 5198 | 0.468 | 7.17 | 7.079 | 7.265 | 7.16 | 7.031 | 7.162 | 0.09 | |
| 213330_s_at | BE886580 | STIP1 | 10963 | −0.22 | 9.21 | 9.333 | 9.264 | 9.338 | 9.017 | 8.931 | 0.03 | |
| 213333_at | AL520774 | MDH2 | 4191 | 0.128 | 5.29 | 5.112 | 5.281 | 5.332 | 5.387 | 5.319 | −0.104 | |
| 213349_at | AI934469 | TMCC1 | 23023 | 1.16 | 6.61 | 6.301 | 6.278 | 6.292 | 7.167 | 7.12 | −0.171 | |
| 213351_s_at | AB018322 | TMCC1 | 23023 | 1.178 | 6.39 | 6.377 | 6.439 | 6.202 | 7.001 | 6.871 | −0.062 | |
| 213352_at | AB018322 | TMCC1 | 23023 | 1.211 | 4.16 | 4.15 | 4.202 | 4.2 | 4.632 | 4.638 | 0.044 | |
| 213376_at | AI656706 | ZBTB1 | 22890 | 0.125 | 7.66 | 7.741 | 7.708 | 7.645 | 7.87 | 7.894 | −0.022 | |
| 213388_at | H15535 | PDE4DIP | 9659 | −0.38 | 5.12 | 5.259 | 5.678 | 5.35 | 5.082 | 4.936 | 0.326 | |
| 213391_at | AI669947 | DPY19L4 | 286148 | −0.47 | 7.95 | 7.951 | 7.639 | 7.538 | 7.7 | 7.828 | −0.359 | |
| 213397_x_at | AI761728 | RNASE4 | 6038 | −0.79 | 3.91 | 3.871 | 3.628 | 3.481 | 3.442 | 3.281 | −0.336 | |
| 213418_at | NM_002155 | HSPA6 | 3310 | 0.024 | 3.27 | 3.265 | 3.238 | 3.252 | 3.019 | 3.277 | −0.022 | |
| 213419_at | U62325 | APBB2 | 323 | 1.111 | 7.19 | 6.891 | 6.857 | 6.64 | 7.829 | 7.898 | −0.289 | |
| 213422_s_at | AW888223 | MXRA8 | 54587 | −0.09 | 3.14 | 3.097 | 3.063 | 2.957 | 3.077 | 3.089 | −0.11 | |
| 213426_s_at | AA15011O | CAV2 | 858 | 0.208 | 3.58 | 3.839 | 3.63 | 3.204 | 3.544 | 3.848 | −0.291 | |
| 213445_at | D63484 | 2C3H3 | 23144 | 0.059 | 4 | 4.313 | 4.155 | 4.08 | 4.448 | 4.586 | −0.037 | |
| 213466_at | BE965869 | RAB40C | 57799 | −0.23 | 4 | 3.574 | 3.883 | 3.78 | 3.87 | 3.542 | 0.044 | |
| 213481_at | N92920 | S10DA13 | 6284 | 0.235 | 4.07 | 3.845 | 3.811 | 3.954 | 3.983 | 4.389 | −0.076 | |
| 213487_at | AI762811 | MAP2K2 | 5605 | 0.125 | 2.95 | 2.697 | 2.699 | 2.679 | 2.902 | 2.78 | −0.134 | |
| 213490_s_at | AT762811 | MAP2K2 | 5605 | 0.023 | 4.36 | 4.302 | 4.276 | 4.42 | 4.625 | 4.676 | 0.017 | |
| 213492_at | X06268 | COL2A1 | 1280 | −0.36 | 3.1 | 3.047 | 3.082 | 2.949 | 3.101 | 2.953 | −0.06 | −0.049 |
| 213509_x_at | AW157619 | CES2 | 8824 | −0.19 | 5.25 | 5.216 | 3.148 | 4.834 | 5.603 | 5.255 | −0.24 | 0.198 |
| 213535_s_at | AA910614 | UBE2I | 7329 | −0.01 | 10.1 | 10.06 | 10.18 | 10.13 | 10.03 | 10.01 | 0.051 | −0.084 |
| 213536_s_at | AA910614 | UBE2I | 7329 | −0.12 | 3.51 | 3.658 | 3.684 | 3.55 | 3.791 | 3.561 | 0.032 | 0.091 |
| 213545_x_at | BE962615 | SNX3 | 8724 | −0.46 | 8.56 | 8.567 | 8.733 | 8.73 | 8.56 | 8.631 | 0.169 | 0.033 |
| 213551_x_at | AI744229 | PCGF2 | 7703 | −0.42 | 4.67 | 4.674 | 4.852 | 4.723 | 4.611 | 4.812 | 0.117 | 0.041 |
| 213559_s_at | BF223401 | ZNF467 | 168544 | −0.06 | 3.08 | 2.962 | 2.944 | 3.053 | 3.146 | 2.968 | −0.021 | 0.038 |
| 213602_s_at | AA401885 | MMP11 | 4320 | −0.02 | 3.25 | 3.294 | 3.395 | 3.323 | 3.373 | 3.25 | 0.085 | 0.037 |
| 213608_s_at | AI220627 | SRRD | 402055 | 0.668 | 6.72 | 6.809 | 6.675 | 6.878 | 6.807 | 6.75 | 0.01 | 0.012 |
| 213636_at | AB028968 | KIAA1045 | 23349 | −0.08 | 3.18 | 2.658 | 2.792 | 2.808 | 2.86 | 2.842 | −0.119 | −0.068 |
| 213549_at | AA524053 | SFRS7 | 6432 | 0.192 | 8.53 | 8.386 | 8.369 | 8.344 | 8.586 | 8.461 | −0.103 | 0.064 |
| 213656_s_at | BF593594 | KLC1 | 3831 | 0.44 | 7.61 | 7.409 | 7.581 | 7.57 | 7.904 | 7.925 | 0.064 | 0.403 |
| 213681_at | AW512817 | CYHR1 | 50626 | 0.007 | 4.12 | 4.068 | 4.342 | 4.015 | 4.566 | 4.531 | 0.086 | 0.456 |
| 213688_at | N25325 | CALM1 /// CALM2 /// | 801 /// 805 /// 808 | 0.149 | 3.93 | 4.485 | 4.238 | 4.295 | 4.269 | 5.045 | 0.06 | 0.45 |
| | | CALM3 | | | | | | | | | | |
| 213708_s_at | N40555 | MLX | 6945 | 0.479 | 8.11 | 8.06 | 8.074 | 8.079 | 8.624 | 8.677 | −0.01 | 0.564 |
| 213741_s_at | BF575685 | KPNA1 | 3836 | −0.01 | 7.05 | 6.769 | 6.957 | 6.784 | 6.675 | 6.795 | −0.038 | −0.174 |
| 213849_s_at | AA974416 | PPP2R2B | 5521 | 0.938 | 2.96 | 3.288 | 3.273 | 3.247 | 3.221 | 3.34 | 0.138 | 0.158 |
| 213858_at | BE350026 | ZNF250 | 58500 | 0.056 | 4.04 | 3.821 | 3.84 | 3.844 | 4.078 | 3.988 | −0.086 | 0.105 |
| 213871_s_at | AA523444 | C6orf108 | 10591 | −0.23 | 3.26 | 3.238 | 3.28 | 3.082 | 3.397 | 3.103 | −0.067 | 0.002 |
| 213889_at | AI742901 | PIGL | 9487 | −0.17 | 4.36 | 4.457 | 4.233 | 4.334 | 4.842 | 4.599 | −0.127 | 0.311 |
| 213910_at | AW770896 | IGFBP7 | 3490 | −0.29 | 2.99 | 2.838 | 2.882 | 2.892 | 2.796 | 2.745 | −0.029 | −0.145 |
| 213917_at | BE465829 | PAX8 | 7849 | −0.03 | 3.02 | 3.023 | 2.921 | 2.898 | 3.115 | 3.062 | −0.11 | 0.069 |
| 213927_at | AV753204 | MAP3K9 | 4293 | 0.305 | 5.29 | 4.939 | 4.936 | 5.044 | 5.203 | 5.213 | −0.124 | 0.094 |
| 213941_x_at | AI970731 | RPS7 | 6201 | −0.01 | 12.4 | 12.44 | 12.42 | 12.31 | 12.42 | 12.38 | −0.065 | −0.027 |
| 213942_at | AL134303 | MEGF6 | 1953 | −0.07 | 3.71 | 3.642 | 3.525 | 3.637 | 3.701 | 3.816 | −0.094 | 0.084 |
| 213969_x_at | BF683426 | RPL29 /// RPL29P4 | 387101 /// 6159 | −0.12 | 12.4 | 12.37 | 12.41 | 12.44 | 12.45 | 12.49 | 0.026 | 0.076 |
| 213982_s_at | BG107203 | RABGAP1L | 9910 | −0.08 | 4.06 | 4.42 | 4.023 | 4.41 | 4.091 | 3.743 | −0.023 | −0.323 |
| 213985_s_at | H45660 | C19orf6 | 91304 | 0.009 | 3.25 | 3.312 | 3.502 | 3.232 | 3.188 | 3.164 | 0.084 | −0.107 |
| 213986_s_at | AI805266 | C19orf6 | 91304 | −0.08 | 4.2 | 4.223 | 4.292 | 4.322 | 4.202 | 4.621 | 0.096 | 0.2 |
| 214026_s_at | AI860246 | SPRED2 | 200734 | 0.014 | 2.77 | 2.853 | 2.678 | 2.884 | 2.95 | 2.849 | −0.033 | 0.086 |
| 214040_s_at | BE675337 | GSN | 2934 | −0.34 | 5.13 | 5.217 | 5.1 | 4.827 | 5.381 | 5.265 | −0.209 | 0.151 |
| 214047_s_at | AI913365 | MBD4 | 8930 | 0.331 | 9.02 | 8.796 | 8.777 | 8.65 | 8.907 | 8.948 | −0.194 | 0.019 |
| 214048_at | AI953365 | MBD4 | 8930 | −0.23 | 5.25 | 5.393 | 5.486 | 5.417 | 5.741 | 5.708 | 0.129 | 0.402 |
| 254061_at | AI017564 | WDR67 | 93594 | 0.104 | 5.96 | 6.209 | 6.039 | 5.901 | 5.964 | 5.843 | −0.116 | −0.182 |
| 214080_x_at | AI815793 | PRKCSH | 5589 | 0.076 | 7.38 | 7.39 | 7.466 | 7.384 | 7.536 | 7.582 | 0.042 | 0.176 |
| 214099_s_at | AK001619 | PDE4DIP | 9659 | 0.164 | 4.69 | 4.554 | 4.947 | 4.283 | 4.774 | 4.731 | −0.008 | 0.129 |
| 214129_at | AI821791 | PDE4DIP | 9659 | 0.171 | 7.21 | 7.386 | 7.281 | 7.32 | 7.684 | 7.546 | 0.004 | 0.319 |
| 214130_s_at | AI821791 | PDE4DIP | 9659 | 0.295 | 5.39 | 5.054 | 5.422 | 5.258 | 5.468 | 5.087 | 0.119 | 0.056 |
| 214134_at | BF939689 | C2orf55 | 343990 | −0.02 | 2.86 | 2.921 | 3.012 | 2.945 | 2.909 | 3.061 | 0.087 | |
| 214141_x_at | BF033354 | SFRS7 | 6432 | 0.538 | 10.8 | 10.79 | 10.58 | 10.78 | 10.63 | 10.54 | −0.112 | |
| 214164_x_at | BF752277 | CA12 | 771 | −0.15 | 9.44 | 9.197 | 9.122 | 9.168 | 9.145 | 9.315 | −0.172 | |
| 214177_s_at | AI935162 | PBXIP1 | 57326 | −0.21 | 5.05 | 4.904 | 5.156 | 4.75 | 4.754 | 4.896 | −0.021 | |
| 214239_x_at | AI560455 | PCGF2 | 7703 | −0.25 | 6.35 | 6.358 | 6.405 | 6.446 | 6.429 | 6.284 | 0.07 | |
| 214310_s_at | AI767884 | ZFPL1 | 7542 | 0.103 | 4.88 | 4.786 | 4.613 | 4.978 | 4.819 | 4.993 | −0.038 | |
| 214311_at | AI767884 | ZFPL1 | 7542 | 0.015 | 3.1 | 3.379 | 3.075 | 3.398 | 3.46 | 3.287 | −0.004 | |
| 214327_x_at | AI888178 | TPT1 | 7178 | −0.08 | 12.4 | 12.37 | 12.45 | 12.43 | 12.29 | 12.37 | 0.035 | |
| 214328_s_at | R01140 | HSP90AA1 | 3320 | −0.3 | 12.5 | 12.56 | 12.51 | 12.6 | 12.11 | 12.13 | 0.002 | |
| 214335_at | AI669349 | RPL18 | 6141 | −0.06 | 3.8 | 3.527 | 3.573 | 3.602 | 4.321 | 3.853 | −0.077 | |
| 214336_s_at | AI621079 | COPA | 1314 | −0.55 | 6.78 | 6.742 | 6.722 | 6.716 | 6.548 | 6.716 | −0.042 | |
| 214337_at | AI621079 | COPA | 1314 | −0.02 | 3.06 | 3.023 | 3.182 | 2.984 | 3.163 | 3.271 | 0.044 | |
| 214338_at | AL050381 | DNAJB12 | 54788 | −0.33 | 4.38 | 4.418 | 4.264 | 4.409 | 4.367 | 4.411 | −0.063 | |
| 214351_x_at | AA789278 | RPL13 | 6137 | −0.04 | 12.3 | 12.21 | 12.33 | 12.22 | 12.14 | 12.15 | 0.041 | |
| 214359_s_at | AI218219 | HSP90AB1 | 3326 | −0.81 | 10.6 | 10.68 | 10.53 | 10.57 | 9.934 | 10.04 | −0.102 | |
| 214391_x_at | AI762344 | PTGER1 | 5731 | 0.213 | 3.36 | 3.348 | 3.49 | 3.688 | 3.741 | 3.511 | 0.236 | |
| 214394_x_at | AI613383 | EEF1D | 1936 | 0.012 | 11.5 | 11.46 | 11.44 | 11.47 | 11.58 | 11.59 | −0.031 | |
| 214395_x_at | AI335509 | EEF1D | 1936 | 0.396 | 5.75 | 6.114 | 5.935 | 6.007 | 5.808 | 5.959 | 0.04 | |
| 214430_at | NM_000169 | GLA | 2717 | −0.16 | 8.32 | 8.266 | 8.485 | 8.466 | 8.184 | 8.414 | 0.183 | |
| 214482_at | NM_006977 | ZBTB25 | 7597 | 0.167 | 5.18 | 4.94 | 4.983 | 5.093 | 4.981 | 4.95 | −0.022 | |
| 214494_s_at | NM_005200 | SPG7 | 6687 | −0.39 | 7.62 | 7.613 | 7.655 | 7.468 | 7.68 | 7.625 | −0.054 | |
| 214516_at | NM_003544 | HIST1H4A /// | 121504 /// 554313 | 0.08 | 3.08 | 3.022 | 3.25 | 3.122 | 3.153 | 3.224 | 0.136 | 0.1444 |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 214528_s_at | NM_013951 | PAX8 | 7849 | 0.174 | 2.51 | 2.802 | 2.515 | 2.542 | 2.753 | 2.441 | −0.128 | −0.059 |
| 214536_at | NM_020427 | SLURP1 | 57152 | 0.088 | 3.18 | 2.962 | 2.872 | 3.204 | 2.839 | 3.191 | −0.031 | −0.054 |
| 214544_s_at | NM_003825 | SNAP23 | 8773 | −0.61 | 1.98 | 4.031 | 4.27 | 4.144 | 3.682 | 3.913 | 0.203 | −0.206 |
| 214550_s_at | AFI45029 | TNPO3 | 23534 | 0.019 | 7.31 | 7.229 | 7.234 | 7.248 | 7.201 | 7.129 | −0.031 | −0.107 |
| 214600_at | AW771935 | TFAD1 | 7003 | −0.01 | 5.47 | 5.381 | 4.923 | 4.924 | 5.051 | 5.208 | −0.497 | −0.293 |
| 234606_s_at | AJ000098 | EYA1 | 2138 | −0.03 | 2.99 | 2.964 | 2.928 | 3.082 | 3.038 | 2.919 | 0.027 | 8E−04 |
| 214634_at | AL523073 | HIST1H4A /// | 121504 /// 554313 | 0.079 | 3.27 | 3.459 | 3.436 | 3.28 | 3.875 | 3.873 | −0.008 | 0.508 |
| | | HIST1H4B /// | /// 8294 /// 8359 /// | | | | | | | | | |
| | | HIST1H4C /// | 8360 /// 8361 /// | | | | | | | | | |
| | | HIST1H4D /// | 8362 /// 8363 /// | | | | | | | | | |
| | | HIST1H4E /// | 8364 /// 8365 /// | | | | | | | | | |
| | | HIST1H4F /// | 8366 /// 8367 /// | | | | | | | | | |
| | | HIST1H4H /// | 8368 /// 8370 | | | | | | | | | |
| | | HIST1H4I /// | | | | | | | | | | |
| | | HIST1H4J /// | | | | | | | | | | |
| | | HIST1H4K /// | | | | | | | | | | |
| | | HIST1H4L /// | | | | | | | | | | |
| | | HIST2H4A /// | | | | | | | | | | |
| | | HIST2H4B /// | | | | | | | | | | |
| | | HIST4H4 | | | | | | | | | | |
| 214692_s_at | AL041139 | JRK | 8629 | −0.29 | 5.34 | 5.163 | 5.196 | 5.225 | 5.342 | 5.347 | −0.041 | 0.093 |
| 214721_x_at | AL162074 | CDC42EP4 | 23580 | 0.04 | 4.99 | 4.851 | 4.822 | 4.803 | 5.014 | 5.137 | −0.109 | 0.154 |
| 214743_at | BE046521 | CUX1 | 1523 | 0.008 | 8.43 | 8.33 | 8.278 | 8.193 | 6.382 | 8.443 | −0.142 | 0.035 |
| 214746_s_at | BE549732 | ZNF467 | 168544 | −0.13 | 6.62 | 5.588 | 6.022 | 5.839 | 6.071 | 5.976 | −0.173 | −0.079 |
| 214748_at | US0529 | N4BP2L2 | 10443 | 0.196 | 4.94 | 4.861 | 4.964 | 4.979 | 5.46 | 5.47 | 0.069 | [symbol] |
| 214753_3t | AW084068 | N4BP2L2 | 10443 | 0.185 | 6.93 | 6.935 | 7.02 | 6.878 | 7.245 | 7.243 | 0.017 | |
| 214760_at | AL049942 | 2NF337 | 26152 | 0.206 | 5.13 | 5.085 | 5.047 | 5.315 | 5.363 | 5.143 | 0.075 | |
| 214818_at | AF007146 | CCDC57 | 284001 | −0.04 | 3.56 | 3.696 | 3.393 | 3.733 | 3.618 | 3.475 | −0.064 | |
| 214827_at | AL031680 | PARD6B | 84612 | 0.066 | 5.08 | 5.138 | 4.941 | 5.313 | 6.182 | 5.454 | 0.016 | |
| 214882_s_at | BG254869 | SFRS2 | 6427 | 0.175 | 10.4 | 10.37 | 10.36 | 10.52 | 10.19 | 10.14 | 0.041 | |
| 214894_x_at | AK023285 | MACF1 | 23499 | −0.97 | 6.38 | 5.943 | 5.996 | 6.099 | 5.939 | 5.897 | −0.113 | |
| 214925_s_at | AK026484 | SPTAN1 | 6709 | −0.01 | 3.99 | 3.812 | 3.743 | 3.992 | 3.487 | 3.604 | −0.031 | |
| 214926_at | AK026484 | SPTAN1 | 6709 | −0 | 3.15 | 2.979 | 2.992 | 2.915 | 2.882 | 2.873 | −0.111 | |
| 214953_s_at | X06989 | APP | 351 | −0.57 | 6.8 | 6.831 | 6.851 | 6.67 | 6.64 | 6.882 | −0.057 | |
| 214969_at | AF2S1442 | MAP3K9 | 4293 | −0.04 | 2.95 | 3.148 | 3.194 | 3.528 | 3.33 | 3.165 | 0.31 | |
| 214976_at | AI554467 | RPL13 | 6137 | −0.04 | 4.04 | 3.771 | 3.67 | 3.772 | 3.583 | 3.821 | −0.182 | |
| 215005_at | AV723666 | NECAB2 | 54550 | −0.16 | 4.14 | 4.143 | 4.077 | 3.963 | 3.985 | 4.317 | −0.121 | |
| 215046_at | AL133053 | C2orf67 | 151050 | 0.137 | 2.97 | 3.289 | 3.26 | 3.078 | 3.242 | 3.197 | 0.038 | |
| 215069_at | AK025065 | NMT2 | 9397 | 0.03 | 3.26 | 3.308 | 3.091 | 3.217 | 3.524 | 3.443 | −0.131 | |
| 215092_s_at | AJ005683 | NFAT5 | 10725 | −0.18 | 5.47 | 5.361 | 5.46 | 5.564 | 5.099 | 5.247 | 0.095 | |
| 215157_x_at | AI734929 | PABPC1 | 26986 | −0.01 | 12.5 | 12.47 | 12.47 | 12.48 | 12.57 | 12.53 | −0.028 | |
| 215184_at | AK026801 | DAPK2 | 23604 | −0.12 | 3.26 | 3.256 | 3.138 | 3.382 | 3.222 | 3.655 | 0.004 | |
| 215194_at | AF035594 | PRKCA | 5578 | 0.084 | 2.98 | 3.015 | 2.865 | 2.977 | 3.106 | 3.357 | −0.078 | |
| 215195_at | AF035594 | PRKCA | 5578 | 0.058 | 4.38 | 3.873 | 4.062 | 3.793 | 4.419 | 4.233 | −0.198 | |
| 215205_x_at | S83390 | NCOR2 | 9612 | 0.059 | 3.13 | 3.117 | 3.035 | 3.364 | 3.13 | 3.296 | 0.074 | |
| 215222_x_at | AK023406 | MACF1 | 23499 | −0.18 | 5.92 | 5.744 | 5.89 | 5.945 | 5.689 | 5.817 | 0.084 | |
| 215231_at | AU144309 | PRKAG2 | 51422 | 0.022 | 3.04 | 3.1111 | 2.998 | 2.986 | 3.284 | 3.091 | −0.083 | |
| 215233_at | AA351360 | JMJD6 | 23210 | −0.05 | 2.89 | 2.869 | 2.561 | 2.861 | 2.93 | 2.863 | −0.17 | |
| 215235_at | AL110273 | SPTAN1 | 6709 | 0.629 | 5.24 | 5.078 | 5.236 | 5.592 | 5.732 | 5.583 | 0.254 | |
| 215240_at | AI189839 | ITGB3 | 3690 | 0.174 | 2.81 | 2.865 | 2.545 | 2.778 | 2.78 | 2.733 | −0.178 | |
| 215270_at | U94354 | LFNG | 3955 | 0.121 | 2.94 | 3.318 | 2.964 | 2.867 | 3.354 | 3.399 | −0.212 | |
| 215337_at | AK022508 | MED24 | 9862 | 0.065 | 3.1 | 3.083 | 3.18 | 3.188 | 3.2 | 3.114 | 0.092 | 0.065 |
| 215342_s_at | AB019490 | RABGAP1L | 9910 | −0.08 | 4.78 | 4.708 | 4.802 | 4.655 | 4.912 | 5.171 | −0.016 | 0.298 |
| 215374_at | AK024849 | PAPOLA | 10914 | −0.19 | 3.67 | 3.371 | 3.405 | 3.275 | 3.271 | 3.418 | −0.181 | −0.175 |
| 215377_at | AK024129 | CTBP2 | 1488 | −0.15 | 3.71 | 3.742 | 3.537 | 3.801 | 3.626 | 3.722 | −0.059 | −0.054 |
| 215548_s_at | AB020724 | SCFD1 | 23256 | 0.246 | 8.62 | 8.659 | 8.678 | 8.813 | 8.459 | 8.533 | 0.107 | −0.143 |
| 215575_at | AU157078 | PDE4DIP | 9659 | −0.04 | 3.19 | 2.946 | 3.318 | 3.156 | 3.204 | 3.299 | 0.169 | 0.184 |
| 215584_at | AK022679 | HECW1 | 23072 | 0.126 | 3.46 | 3.496 | 3.376 | 3.357 | 3.099 | 3.212 | −0.111 | −0.321 |
| 215517_at | AU145711 | LOC26010 | 26010 | −0.22 | 2.8 | 2.644 | 2.737 | 2.747 | 2.704 | 2.707 | 0.02 | −0.017 |
| 215631_s_at | AL0S0G08 | BRMS1 | 25855 | 0.297 | 6.7 | 6.721 | 6.823 | 6.769 | 6.31 | 7.087 | 0.083 | 0.286 |
| 215688_at | AL359931 | RASGRF1 | 5923 | −0.12 | 3.23 | 3.064 | 3.305 | 3.259 | 3.047 | 3.214 | 0.133 | −0.018 |
| 215728_at | AL031848 | ACOT7 | 11332 | 0.388 | 5.95 | 5.834 | 5.403 | 5.581 | 5.68 | 5.682 | −0.398 | −0.209 |
| 215732_s_at | AK023924 | DTX2 /// | 100134197 // | −0.11 | 4.08 | 4.064 | 4.059 | 3.946 | 3.926 | 4.122 | −0.072 | −0.05 |
| | | LOC100134197 | 113878 | | | | | | | | | |
| 215743_at | AL134483 | NMT2 | 9397 | −0.08 | 3.07 | 3.157 | 3.65 | 3.379 | 3.252 | 3.14 | 0.399 | 0.081 |
| 215852_x_at | AK022023 | C20orftL17 | 140710 | −0.26 | 3.16 | 3.191 | 3.175 | 3.179 | 3.236 | 3.251 | 0.003 | 0.07 |
| 215867_x_at | AL050025 | CA12 | 771 | −0.11 | 9.26 | 9.064 | 9.034 | 9.125 | 9.076 | 9.247 | −0.082 | 5E−04 |
| 215912_at | AA758795 | GNAO1 | 2775 | 0.054 | 3.2 | 3.66 | 3.266 | 3.267 | 3.435 | 3.337 | −0.162 | −0.042 |
| 215938_s_at | AK001290 | PLA2G6 | 8398 | −0.12 | 3.38 | 3.008 | 3.185 | 3.146 | 3.224 | 3.272 | −0.025 | 0.057 |
| 215980_s_at | AF052128 | IGHMBP2 | 3508 | −0.14 | 3.8 | 3.828 | 3.784 | 3.754 | 4.092 | 3.957 | −0.044 | 0.211 |
| 215991_s_at | AU121504 | KIAA0090 | 23065 | 0.063 | 2.98 | 2.987 | 2.808 | 2.99 | 2.945 | 2.935 | −0.087 | −0.046 |
| 216105_x_at | X86428 | PPP2R4 | 5524 | −0.1 | 6.32 | 6.425 | 6.518 | 6.132 | 6.45 | 6.384 | −0.047 | 0.045 |
| 216261_at | AI151479 | ITGB3 | 3690 | 0.036 | 3.07 | 3.174 | 3.05 | 2.947 | 2.824 | 3.105 | −0.122 | −0.156 |
| 216309_x_at | AF072467 | JPX | 8629 | −0.37 | 5.59 | 5.862 | 5.797 | 5.765 | 6.085 | 5.892 | 0.096 | 0.263 |
| 216364_s_at | AJ001550 | AFF2 | 2334 | −0 | 2.66 | 2.757 | 2.779 | 2.588 | 2.676 | 2.739 | −0.025 | −5E−04 |
| 216382_s_at | U80756 | MLL2 | 8085 | −0.18 | 3.72 | 3.495 | 3.501 | 3.547 | 3.506 | 3.479 | −0.085 | −0.116 |
| 216407_at | U25801 | VAC14 | 55697 | 0.267 | 3.86 | 3.898 | 3.861 | 4.059 | 4.073 | 3.689 | 0.08 | 0.001 |
| 216501_at | U25801 | VAC14 | 55697 | −0.09 | 2.81 | 2.887 | 2.706 | 2.624 | 2.865 | 2.832 | −0.183 | 5E−04 |
| 216520_s_at | AF072098 | TPT1 | 7178 | −0.07 | 13 | 12.95 | 12.95 | 12.97 | 12.88 | 12.88 | 0.006 | −0.074 |
| 216533_at | AL122056 | PCCA | 5095 | −0.06 | 2.55 | 2.544 | 2.593 | 2.54 | 2.725 | 2.495 | 0.021 | 0.064 |
| 216570_x_at | AL096829 | LOC100131713 /// | 100131713 /// | −0.51 | 9.96 | 9.98 | 9.892 | 9.915 | 9.592 | 9.617 | −0.069 | −0.367 |
| | | LOC283412 /// | 283412 /// 284064 | | | | | | | | | |
| | | LOC284064 /// | /// 387101 /// | | | | | | | | | |
| | | LOC391019 /// | 391019 /// 6159 /// | | | | | | | | | |
| | | LOC643531 /// | 643531 /// 647285 | | | | | | | | | |
| | | LOC647285 /// | /// 728820 | | | | | | | | | |
| | | LOC728820 /// RPL29 | | | | | | | | | | |
| | | /// RPL29P4 | | | | | | | | | | |
| 216624_s_at | Z69744 | MLL | 4297 | −0.25 | 3.3 | 3.292 | 3.228 | 3.166 | 3.131 | 2.902 | −0.099 | −0.279 |
| 216678_at | AK000773 | IFT122 | 55764 | 0.024 | 4.65 | 4.379 | 4.344 | 4.173 | 4.051 | 3.961 | −0.257 | −0.509 |
| 216697_at | AL161955 | TRIO | 7204 | −0.02 | 2.77 | 2.948 | 2.953 | 2.896 | 2.913 | 2.969 | 0.0 | |
| 216700_at | AL161955 | TRIO | 7204 | −0.08 | 3.65 | 3.267 | 3.35 | 3.394 | 3.206 | 3.235 | −0.0 | |
| 216747_at | AK024871 | APBB2 | 323 | 0.168 | 3.68 | 3.491 | 3.36 | 3.348 | 3.662 | 3.784 | −0.2 | |
| 216750_at | AK024871 | APBB2 | 323 | −0.25 | 3.42 | 3.732 | 3.34 | 3.335 | 3.38 | 3.56 | −0.2 | |
| 216845_x_at | U80756 | MLL2 | 8085 | −0.18 | 3.4 | 3.824 | 3307 | 3.469 | 3.553 | 3.443 | −0.1 | |
| 216867_s_at | X03795 | PDGFA | 5154 | 0.602 | 4.71 | 4.421 | 4.45 | 4.791 | 5.536 | 5.369 | 0.0 | |
| 216880_at | Y15571 | RAD51L1 | 5890 | 0.027 | 4.98 | 4.877 | 5.108 | 5.196 | 4.16 | 4.473 | 0.2 | |
| 216944_x_at | U23850 | ITPR1 | 3708 | −0.07 | 3.13 | 3.076 | 3.298 | 2.983 | 3.346 | 3.052 | 0.0 | |
| 216952_s_at | M94363 | LMNB2 | 84823 | −0.03 | 4.94 | 4.728 | 4.801 | 5.079 | 4.77 | 4.624 | 0.1 | |
| 216971_s_at | 254367 | PLEC1 | 5339 | −0.01 | 3.5 | 3.845 | 3.638 | 3.787 | 3.409 | 3.58 | 0.0 | |
| 216988_s_at | L48722 | PTP4A2 | 8073 | 0.387 | 8.98 | 8.919 | 8.804 | 8.81 | 8.861 | 8.793 | −0 | |
| 217005_at | M28219 | LDLR | 3949 | −0.16 | 3.53 | 3.475 | 3.52 | 3.376 | 4.031 | 3.369 | −0.0 | |
| 217025_s_at | AL110225 | DBN1 | 1627 | 0.173 | 3.81 | 3.728 | 3.68 | 3.688 | 3.597 | 3.93 | −0.0 | |
| 217103_at | M28219 | LDLR | 3949 | 0.057 | 2.98 | 3.02 | 2.742 | 3.069 | 3.026 | 2.962 | −0.0 | |
| 217118_s_at | AK025608 | C22orf9 | 23313 | 0.324 | 6.57 | 6.503 | 6.55 | 6.379 | 6.821 | 6.948 | −0.0 | |
| 217124_at | AL136792 | IQCE | 23288 | 0.151 | 3.21 | 3.278 | 3.153 | 3.29 | 3.123 | 3.309 | −0.0 | |
| 217144_at | X04801 | LOC648390 /// | 6233 /// 648390 /// | −1.02 | 6.47 | 6.664 | 6.55 | 6.559 | 5.889 | 5.704 | −0.0 | |
| | | RPS27A /// UBB /// | 7314 /// 7316 | | | | | | | | | |
| | | UBC | | | | | | | | | | |
| 217146_at | AF072468 | JRK | 8629 | −0.11 | 3.01 | 2.84 | 2.928 | 3.154 | 3.008 | 2.854 | 0.1 | |
| 217173_s_at | S70123 | LDLR | 3949 | 0.398 | 5.77 | 5.697 | 5.77 | 5.815 | 6.003 | 5.923 | 0.0 | |
| 217174_s_at | AL078616 | APC2 | 10297 | −0.03 | 2.98 | 2.896 | 3.025 | 2.99 | 2.803 | 2.815 | 0.0 | |
| 217183_at | S70123 | LDLR | 3949 | −0.08 | 3.31 | 3.232 | 3.45 | 3.346 | 3.635 | 3.198 | 0.1 | |
| 217262_s_at | BC000059 | CELSR1 | 9620 | 0.067 | 2.9 | 3.093 | 3.145 | 3.049 | 3.399 | 2.949 | 0.0 | |
| 217299_s_at | AK001017 | NBN | 4683 | 0.029 | 7.64 | 7.664 | 7.528 | 7.656 | 7.786 | 7.545 | −0.0 | |
| 217356_s_at | S81916 | PGR1 | 5230 | −0.36 | 10.4 | 10.49 | 10.53 | 10.46 | 10.32 | 10.3 | 0.0 | |
| 217383_at | S81916 | PGK1 | 5230 | 0.21 | 4.68 | 4.784 | 4.916 | 4.744 | 4.496 | 4.47 | 0.096 | −0.251 |
| 217404_s_at | X16468 | COL2A1 | 1280 | −0.31 | 2.71 | 2.76 | 2.754 | 2.994 | 2.816 | 2.776 | 0.138 | 0.06 |
| 217432_s_at | AF179281 | IDS | 3423 | −0.01 | 4.58 | 5.315 | 5.136 | 5.522 | 5.198 | 5.466 | 0.383 | 0.386 |
| 217466_x_at | L48784 | RP52 | 6187 | −0.22 | 10.4 | 10.48 | 10.55 | 10.34 | 10.23 | 10.21 | 0.001 | −0.222 |
| 217489_s_at | S72848 | IL6R | 3570 | 0.101 | 2.81 | 2.996 | 2.797 | 3.029 | 2.945 | 2.772 | 0.008 | −0.046 |
| 217500_at | R27378 | TIAL1 | 7073 | −0.26 | 3.12 | 3.048 | 3.226 | 3.255 | 2.997 | 3.131 | 0.156 | −0.02 |
| 217508_s_at | BE783279 | C18orf25 | 147339 | 0.439 | 4.72 | 4.902 | 4.652 | 4.524 | 5.347 | 5.131 | −0.225 | 0.426 |
| 217539_at | W28849 | C18orf25 | 147339 | 0.109 | 2.7 | 2.553 | 2.554 | 2.738 | 2.659 | 2.756 | 0.018 | 0.08 |
| 217608_at | AW408767 | SFRS12IP1 | 285672 | −0.08 | 3.12 | 3.193 | 3.09 | 3.417 | 2.978 | 2.835 | 0.096 | 0.251 |
| 217618_x_at | AW007988 | HUS1 | 3364 | 0.511 | 4.54 | 4.464 | 4.473 | 4.627 | 4.652 | 4.542 | 0.048 | 0.095 |
| 217622_at | AA018187 | RHBDD3 | 25807 | −0.04 | 5.76 | 5.474 | 5.504 | 5.38 | 5.392 | 5.536 | −0.172 | −0.151 |
| 217635_s_at | AA769006 | POLG | 5428 | −0.02 | 5.37 | 5.404 | 5.544 | 5.351 | 5.406 | 5.471 | 0.062 | 0.053 |
| 217636_at | AA769006 | POLG | 5428 | −0.08 | 2.97 | 2.938 | 2.95 | 2.944 | 2.668 | 3.028 | −0.006 | −0.106 |
| 217669_s_at | AW451230 | AKAP6 | 9472 | 0.229 | 3.07 | 3.132 | 2.966 | 3.236 | 3.256 | 3.672 | −3E−04 | 0.363 |
| 217686_at | BF222916 | PTPN1 | 5770 | 0.02 | 3.84 | 3.702 | 3.49 | 3.761 | 3.593 | 3.374 | −0.144 | −0.285 |
| 217689_at | BG109555 | PTPN1 | 5770 | 0.011 | 3.19 | 3.021 | 2.778 | 2.977 | 2.954 | 3.055 | 0.226 | −0.099 |
| 217722_s_at | NM_016645 | NGRN | 51335 | 0.061 | 11 | 10.85 | 10.9 | 10.91 | 10.83 | 10.83 | −0.01 | −0.086 |
| 217745_s_at | NM_025146 | NAT13 | 80218 | 0.08 | 9.82 | 9.816 | 9.885 | 9.821 | 9.489 | 9.587 | 0.035 | −0.28 |
| 217752_s_at | NM_018235 | CNDP2 | 55748 | 0.239 | 8.98 | 8.909 | 8.911 | 8.849 | 9.061 | 9.114 | −0.064 | 0.145 |
| 217756_x_at | NM_005770 | SERF2 | 10169 | −0.07 | 10.7 | 10.81 | 10.74 | 10.76 | 10.71 | 10.59 | −0.014 | −0.113 |
| 217774_s_at | NM_016404 | HSPC152 | 51504 | −0.27 | 11.9 | 11.91 | 11.8 | 11.81 | 12.05 | 12.04 | −0.097 | 0.144 |
| 217779_s_at | NM_017761 | LOC100132235 /// | 100132235 /// 55629 | 0.036 | 8.95 | 8.998 | 8.837 | 8.758 | 9.568 | 9.549 | −0.178 | 0.583 |
| | | PNRC2 | | | | | | | | | | |
| 217786_at | NM_006109 | PRMT5 | 10419 | 0.507 | 9 | 8.884 | 8.999 | 9.051 | 9.101 | 9.164 | 0.082 | 0.189 |
| 217793_at | AL575337 | RAB11B | 9230 | 0.092 | 3.45 | 3.64 | 3.536 | 3.505 | 3.648 | 3.671 | −0.022 | 0.116 |
| 217830_s_at | AL109658 | NSFL1C | 55968 | −0.08 | 4.55 | 4.497 | 5.301 | 5.088 | 4.749 | 4.274 | 0.673 | −0.011 |
| 217831_s_at | NM_016143 | NSFL1C | 55968 | −0.07 | 5.85 | 5.907 | 5.875 | 5.936 | 5.7 | 6.065 | 0.029 | 0.006 |
| 217868_s_at | NM_016025 | METTL9 | 51108 | −0.26 | 10.4 | 10.49 | 10.35 | 10.43 | 10.22 | 10.18 | −0.067 | −0.256 |
| 217875_s_at | NM_020182 | PMEPA1 | 56937 | 1.055 | 6.72 | 7.047 | 7.04 | 6.888 | 7.38 | 7.248 | 0.079 | 0.429 |
| 217903_at | NM_013403 | STRN4 | 29888 | 0.394 | 4.68 | 4.656 | 4.862 | 4.591 | 4.769 | 4.69 | 0.058 | 0.06 |
| 217907_at | NM_014161 | MRPL18 | 29074 | 0.009 | 9.18 | 9.176 | 9.159 | 9.205 | 8.869 | 8.962 | 0.002 | −0.265 |
| 217909_s_at | BF056105 | MLX | 6945 | 0.167 | 7.14 | 7.062 | 6.924 | 7.005 | 7.442 | 7.434 | −0.135 | 0.339 |
| 217910_x_at | NM_013383 | MLX | 6945 | 0.314 | 7.65 | 7.572 | 7.57 | 7.6 | 7.912 | 8.121 | −0.027 | 0.404 |
| 217911_s_at | NM_004281 | BAG3 | 9531 | −0.59 | 9.84 | 9.656 | 9.746 | 9.793 | 9.677 | 9.94 | 0.02 | 0.06 |
| 217924_at | AL523965 | C6orf106 | 64771 | 0.141 | 4.96 | 4.815 | 4.81 | 5.018 | 5.012 | 5.358 | 0.029 | 0.3 |
| 217925_s_at | NM_022758 | C6orf106 | 64771 | 0.162 | 5.51 | 5.893 | 6.03 | 5.894 | 5.503 | 5.888 | 0.258 | −0.009 |
| 217943_s_at | NM_018067 | MAP7D1 | 55700 | 0.352 | 4.95 | 4.057 | 4.311 | 4.149 | 4.488 | 4.587 | −0.273 | 0.035 |
| 217950_at | NM_015953 | NOSIP | 51070 | 0.231 | 6.93 | 7.2 | 7.111 | 7.192 | 7.279 | 7.393 | 0.084 | 0.269 |
| 217969_at | NM_013265 | C11orf2 | 738 | 0.047 | 8.07 | 7.826 | 8.009 | 7.931 | 8.01 | 8.065 | 0.021 | 0.089 |
| 217980_s_at | NM_017840 | MRPL16 | 54948 | 0.187 | 9.15 | 9.067 | 9.089 | 9.13 | 9.172 | 9.138 | −0. [symbol] | |
| 218016_s_at | NM_018119 | POLR3E | 55718 | 0.381 | 7.8 | 7.681 | 7.732 | 7.722 | 8.017 | 7.902 | −0. [symbol] | |
| 218018_at | AW449022 | PDXK | 8566 | 0.43 | 8.2 | 8.15 | 8.084 | 8.236 | 8.482 | 8.591 | −0. [symbol] | |
| 218019_s_at | NM_021941 | PDXK | 8566 | −0.37 | 6.81 | 6.67 | 6.96 | 6.993 | 6.69 | 6.708 | 0.2 [symbol] | |
| 218022_at | NM_016440 | VRX3 | 51231 | 0.011 | 6.47 | 6.459 | 6.264 | 6.376 | 6.444 | 6.734 | −0.1 [symbol] | |
| 218023_s_at | NM_016605 | FAM53C | 51307 | 0.06 | 6.68 | 6.479 | 6.583 | 6.721 | 6.232 | 6.231 | 0. [symbol] | |
| 218062_x_at | NM_012121 | CDC42EP4 | 23580 | 0.273 | 5.13 | 5.172 | 5.211 | 5.398 | 5.407 | 5.616 | 0. [symbol] | |
| 218063_s_at | AF099664 | CDC42EP4 | 23580 | −0 | 3.13 | 3.043 | 3.079 | 3.033 | 3.055 | 3.06 | −0. [symbol] | |
| 218074_at | NM_016062 | FAM96B | 51647 | 0.141 | 9.34 | 9.28 | 9.242 | 9.255 | 9.485 | 9.49 | −0. [symbol] | |
| 218099_at | NM_018469 | TEX2 | 55852 | 0.076 | 6.71 | 6.75 | 6.644 | 6.8 | 6.798 | 6.677 | −0. [symbol] | |
| 218132_s_at | NM_024075 | TSEN34 | 79042 | −0.08 | 8.52 | 8.372 | 8.448 | 8.477 | 8.493 | 8.526 | 0. [symbol] | |
| 218136_s_at | NM_018579 | SLC25A37 | 51312 | −0.33 | 4.03 | 3.849 | 4.13 | 3.959 | 3.861 | 3.919 | 0.1 [symbol] | |
| 218138_at | NM_018848 | MKKS | 8195 | 0.24 | 7.68 | 7.583 | 7.684 | 7.734 | 7.706 | 7.75 | 0. [symbol] | |
| 218141_at | NM_022066 | UBE2O | 63893 | 0.341 | 4.46 | 4.359 | 4.41 | 4.494 | 4.312 | 4.758 | 0. [symbol] | |
| 218145_at | NM_021158 | TRIB3 | 57761 | 1.079 | 7.7 | 7.871 | 8.174 | 7.866 | 7.941 | 7.66 | 0.2 [symbol] | |
| 218148_at | NM_025082 | CENPT | 80152 | −0.05 | 3.56 | 3.59 | 3.489 | 3.208 | 3.396 | 3.15 | −0.2 [symbol] | |
| 218169_at | NM_018052 | VAC14 | 55697 | 0.509 | 5.1 | 4.765 | 4.661 | 4.826 | 5.311 | 5.367 | −0.1 [symbol] | |
| 218181_s_at | NM_017792 | MAP4K4 | 9448 | −0.26 | 5.47 | 5.399 | 5.362 | 5.358 | 5.503 | 5.245 | −0. [symbol] | |
| 218195_at | NM_024573 | C6orf211 | 79624 | 0.194 | 10.9 | 11.03 | 10.96 | 10.85 | 10.36 | 10.38 | −0. [symbol] | |
| 218197_s_at | NM_018002 | OXR1 | 55074 | −0.46 | 7.61 | 7.459 | 7.747 | 7.654 | 7.091 | 7.128 | 0.1 [symbol] | |
| 218233_s_at | NM_017601 | PRICKLE4 /// TOMM6 | 100188893 /// 29964 | 0.104 | 11.9 | 11.94 | 11.86 | 11.89 | 11.97 | 11.94 | −0. [symbol] | |
| 218235_s_at | NM_016037 | UTP11L | 51118 | 0.265 | 8.62 | 8.551 | 8.526 | 8.602 | 8.73 | 8.518 | −0. [symbol] | |
| 218246_at | NM_024544 | MUL1 | 79594 | −0.24 | 4.87 | 4.49 | 4.677 | 4.857 | 4.557 | 4.77 | 0. [symbol] | |
| 218265_at | NM_024077 | SEC1SBP2 | 79048 | 0.231 | 5.9 | 5.783 | 5.771 | 5.548 | 5.918 | 6.106 | −0. [symbol] | |
| 218270_at | NM_024540 | MRPL24 | 79590 | 0.008 | 7.93 | 8.008 | 8.021 | 7.933 | 7.84 | 7.768 | 0. [symbol] | |
| 218292_s_at | NM_016203 | PRKAG2 | 51422 | 1.117 | 5.07 | 4.827 | 4.618 | 4.685 | 5.621 | 5.541 | −0.299 | 0.63 |
| 218321_x_at | NM_016086 | STYXL1 | 51657 | 0.209 | 7.7 | 7.632 | 7.648 | 7.553 | 7.329 | 7.429 | −0.067 | −0.289 |
| 218328_at | NM_016035 | COQ4 | 51117 | 0.106 | 6.3 | 6.378 | 6.545 | 6.507 | 7.175 | 7.106 | 0.188 | 0.803 |
| 218343_s_at | NM_012086 | GTF3C3 | 9330 | −0.21 | 7.07 | 7.321 | 7.289 | 7.296 | 7.289 | 7.255 | 0.095 | 0.06 |
| 218347_at | NM_018264 | TYW1 | 55253 | −0.09 | 6.98 | 6.994 | 6.86 | 6.828 | 6.816 | 6.541 | −0.141 | −0.307 |
| 218364_at | NM_017724 | LRRFIP2 | 9209 | 0.924 | 5.73 | 5.857 | 5.793 | 5.592 | 6.019 | 5.875 | −0.102 | 0.152 |
| 218402_s_at | NM_022081 | HPS4 | 89781 | −0.08 | 3.73 | 3.683 | 3.771 | 3.928 | 3.526 | 3.736 | 0.142 | −0.077 |
| 218427_at | NM_006643 | SDCCAG3 | 10807 | 0.831 | 7.22 | 7.138 | 7.337 | 7.25 | 7.68 | 7.637 | 0.115 | 0.48 |
| 218431_at | NM_022067 | C14orf133 | 63894 | −0.34 | 7.07 | 7.015 | 6.931 | 6.991 | 7.056 | 7.031 | −0.083 | −3E−04 |
| 218480_at | NM_021831 | AGBL5 | 60509 | −0.42 | 5.93 | 5.84 | 6.003 | 5.924 | 6.252 | 5.156 | 0.076 | 0.317 |
| 218482_at | NM_020189 | ENY2 | 56943 | 0.432 | 10.6 | 10.59 | 10.65 | 10.69 | 10.88 | 10.76 | 0.069 | 0.216 |
| 218500_at | NM_016647 | C8orf55 | 51337 | −0.29 | 5.89 | 5.342 | 5.749 | 5.684 | 5.468 | 5.547 | 0.101 | −0.108 |
| 218543_s_at | NM_022750 | PARP12 | 64761 | 0.336 | 4.98 | 4.899 | 5.083 | 5.159 | 5.652 | 5.708 | 0.184 | 0.743 |
| 218555_at | NM_013366 | ANAPC2 | 29882 | −0.29 | 5.21 | 5.176 | 5.569 | 5.575 | 5.233 | 6.065 | 0.379 | 0.456 |
| 218561_s_at | NM_020408 | LYRM4 | 57128 | −0.06 | 7.241 | 7.196 | 7.2163 | 7.216 | 7.8301 | 7.7461 | −0.002 | 0.57 |
| 218566_s_at | NM_012124 | CHORDC1 | 26973 | 0.506 | 7.68 | 7.731 | 7.677 | 7.63 | 7.347 | 7.251 | −0.053 | −0.407 |
| 218578_at | NM_024529 | CDC73 | 79577 | 0.283 | 8.33 | 8.154 | 7.979 | 8.062 | 8.116 | 8.122 | −0.222 | −0.123 |
| 218584_at | NM_024549 | TCTN1 | 79600 | −0.63 | 5.65 | 5.692 | 5.57 | 5.596 | 5.369 | 5.236 | −0.09 | −0.37 |
| 218596_at | NM_018201 | TBC1D13 | 54662 | −0.04 | 4.5 | 4.467 | 4.749 | 5.045 | 4.36 | 4.031 | 0.414 | −0.287 |
| 218677_at | NM_020672 | S100A14 | 57402 | −0.3 | 8.55 | 8.71 | 8.882 | 8.779 | 8.947 | 8.872 | 0.202 | 0.281 |
| 218678_at | NM_024609 | NES | 10763 | −0.17 | 4.34 | 4.643 | 4.504 | 4.545 | 3.964 | 3.92 | 0.032 | −0.55 |
| 218680_x_at | NM_016400 | HYPK | 25764 | 0.265 | 9.57 | 9.612 | 9.336 | 9.476 | 9.391 | 9.323 | −0.184 | −0.233 |
| 218763_at | NM_016930 | STX18 | 53407 | 0.268 | 7.15 | 7.042 | 7.111 | 7.128 | 6.972 | 7.075 | 0.024 | −0.072 |
| 218767_at | NM_020385 | REXO4 | 57109 | 0.256 | 6.38 | 6.21 | 6.272 | 6.286 | 6.435 | 6.586 | −0.015 | 0.217 |
| 218810_at | NM_025079 | ZC3H12A | 80149 | 0.086 | 3.84 | 4.091 | 4.274 | 4.341 | 4.777 | 4.457 | 0.343 | 0.653 |
| 218818_at | NM_004468 | PHL3 | 2275 | −0.07 | 3.09 | 3.045 | 3.12 | 2.725 | 3.326 | 3.235 | −0.147 | 0.212 |
| 218830_at | NM_016093 | RPL26L1 | 51121 | 0.451 | 10.5 | 10.38 | 10.41 | 10.41 | 10.58 | 10.53 | −0.006 | 0.143 |
| 218846_at | NM_004830 | MED23 | 9439 | 0.016 | 7.17 | 7.098 | 7.12 | 7.189 | 7.169 | 7.02 | 0.021 | −0.039 |
| 218847_at | NM_006548 | IGF2BP2 | 10644 | 0.453 | 6.27 | 6.17 | 6.135 | 6.107 | 7.149 | 6.992 | −0.1 | 0.849 |
| 218850_s_at | NM_014240 | LIMD1 | 8994 | 0.232 | 3.47 | 3.294 | 3.569 | 3.468 | 3.591 | 3.613 | 0.136 | 0.219 |
| 218914_at | NM_015997 | C1orf66 | 51093 | 0.015 | 5.37 | 5.498 | 5.333 | 5.421 | 5.493 | 5.611 | −0.056 | 0.119 |
| 218954_s_at | AF298153 | BRF2 | 55290 | −0.21 | 4.29 | 4.242 | 4.196 | 4.055 | 4.078 | 4.108 | −0.139 | −0.171 |
| 218955_at | NM_018310 | BRF2 | 55290 | −0.16 | 4.95 | 4.98 | 5.184 | 5.17 | 4.906 | 5.032 | 0.213 | 0.004 |
| 218965_s_at | NM_022830 | TUT1 | 64852 | −0.07 | 3.66 | 3.901 | 3.624 | 3.608 | 3.731 | 3.546 | −0.164 | −0.142 |
| 218966_at | NM_018728 | MYO5C | 55930 | −0.07 | 9.95 | 9.841 | 9.746 | 9.875 | 9.576 | 9.774 | −0.088 | −0.223 |
| 218978_s_at | NM_018586 | SLC25A37 | 51312 | −0.42 | 3.57 | 4.059 | 4.061 | 3.724 | 3.84 | 3.851 | 0.077 | 0.03 |
| 218991_at | NM_022070 | HEATR6 | 63897 | −0.28 | 10.2 | 10.36 | 10.38 | 10.42 | 10.56 | 10.57 | 0.108 | 0.27 |
| 219038_at | NM_024657 | MORC4 | 79710 | 0.134 | 5.58 | 5.916 | 5.89 | 5.783 | 5.813 | 5.694 | 0.089 | 0.006 |
| 219050_s_at | NM_014205 | ZNHIT2 | 741 | 0.31 | 4.23 | 4.309 | 4.256 | 4.374 | 4.997 | 4.891 | 0.045 | 0.674 |
| 219062_s_at | NM_017742 | ZCCHC2 | 54877 | −0.15 | 5.65 | 5.951 | 5.531 | 5.736 | 6.139 | 6.031 | −0. [symbol] | |
| 219076_s_at | NM_018663 | PXMP2 | 5827 | −0.17 | 6.85 | 6.881 | 6.791 | 6.919 | 7.261 | 6.945 | −0 [symbol] | |
| 219107_at | NM_021948 | BCAN | 63827 | −0.1 | 3.56 | 3.636 | 3.433 | 3.383 | 3.619 | 3.329 | −0. [symbol] | |
| 219128_at | NM_017880 | C2orf42 | 54980 | 0.511 | 6.34 | 6.366 | 6.242 | 6.277 | 6.914 | 6.757 | −0. [symbol] | |
| 219156_at | NM_018373 | SYNJ2BP | 55333 | −0.41 | 6.58 | 6.696 | 6.628 | 6.407 | 6.729 | 6.604 | −0 [symbol] | |
| 219172_at | NM_024954 | UBTD1 | 80019 | −0.07 | 2.9 | 3.108 | 2.999 | 3.099 | 3.025 | 3.146 | 0. [symbol] | |
| 219175_s_at | NM_017836 | SLC41A3 | 54946 | −0.15 | 5.59 | 5.644 | 5.55 | 5.712 | 5.642 | 5.636 | 0. [symbol] | |
| 219193_at | NM_018034 | WDR70 | 55100 | 0.046 | 5.92 | 6.234 | 6.204 | 6.25 | 6.424 | 6.223 | 0. [symbol] | |
| 219215_s_at | NM_017767 | SLC39A4 | 55630 | 0.883 | 7.99 | 8.044 | 8.105 | 8.289 | 8.135 | 8.093 | 0. [symbol] | |
| 219217_at | NM_024678 | NARS2 | 79731 | 0.445 | 8.07 | 7.943 | 8.073 | 8.009 | 7.793 | 7.962 | 0. [symbol] | |
| 219221_at | NM_024724 | ZBTB38 | 253461 | 0.299 | 6.43 | 6.511 | 6.661 | 6.58 | 6.549 | 6.582 | 0. [symbol] | |
| 219227_at | NM_024565 | CCNJL | 79616 | 0.009 | 3.14 | 3.607 | 3.256 | 3.492 | 3.447 | 3.696 | −0. [symbol] | |
| 219354_at | NM_018316 | KLHL26 | 55295 | −0.09 | 4.7 | 4.534 | 4.625 | 4.612 | 4.45 | 4.54 | 0. [symbol] | |
| 219357_at | NM_014027 | GTPBP1 | 9567 | 0.287 | 5.27 | 5.043 | 5.188 | 5.179 | 5.277 | 5.755 | 0. [symbol] | |
| 219435_at | NM_025099 | C17orf68 | 80169 | −0.06 | 4.76 | 4.633 | 4.915 | 4.674 | 4.774 | 4.551 | 0. [symbol] | |
| 219456_s_at | AW027923 | RIN3 | 79890 | 0.035 | 2.8 | 3.001 | 2.84 | 2.984 | 2.915 | 2.984 | 0. [symbol] | |
| 219457_s_at | NM_024832 | RIN3 | 79890 | 0.196 | 2.93 | 2.962 | 2.999 | 3.129 | 3.196 | 3.128 | 0 [symbol] | |
| 219459_at | NM_018082 | POLR3B | 55703 | 0.405 | 6.96 | 6.965 | 7.158 | 7.117 | 7.552 | 7.487 | 0. [symbol] | |
| 219468_s_at | NM_017949 | CUEDC1 | 404093 | 0.191 | 3.78 | 4.049 | 4.3 | 4.334 | 4.477 | 4.828 | 0. [symbol] | |
| 219475_at | NM_013370 | OSGIN1 | 29948 | −0.02 | 3.92 | 3.987 | 4.5 | 4.041 | 4.234 | 3.74 | 0. [symbol] | |
| 219489_s_at | NM_017821 | NXN | 64359 | 0.031 | 7.02 | 6.891 | 7.174 | 7.164 | 7.871 | 7.808 | 0. [symbol] | |
| 219495_s_at | NM_013256 | ZNF180 | 7733 | 0.179 | 5.06 | 4.859 | 4.78 | 5.024 | 4.857 | 5.207 | −0. [symbol] | |
| 219500_at | NM_013246 | CLCF1 | 23529 | 0.294 | 4.22 | 4.563 | 4.41 | 4.298 | 4.035 | 4.159 | −0. [symbol] | |
| 219513_s_at | NM_005490 | SH2D3A | 10045 | 0.509 | 2.87 | 3.082 | 2.892 | 3.204 | 3.205 | 3.051 | 0. [symbol] | |
| 219543_at | NM_022129 | PBLD | 64081 | −0.14 | 5.2 | 5.578 | 5.578 | 5.461 | 6.02 | 5.972 | 0. [symbol] | |
| 219572_at | NM_037954 | CADPS2 | 93664 | −0.08 | 5.66 | 5.061 | 5.288 | 5.272 | 4.703 | 5.226 | −0.078 | −0.394 |
| 219577_s_at | NM_019112 | ABCA7 | 10347 | −0.07 | 3.06 | 3.264 | 3.211 | 3.479 | 3.453 | 3.368 | 0.184 | 0.25 |
| 219610_at | NM_022448 | RGNEF | 64283 | −0.54 | 3.69 | 3.608 | 3.647 | 3.479 | 3.408 | 3.662 | −0.085 | −0.113 |
| 219631_at | NM_024937 | LRP12 | 29967 | −0.16 | 3.91 | 4.365 | 3.897 | 3.952 | 3.822 | 4.228 | −0.214 | −0.114 |
| 219677_st | NM_025106 | SPSB1 | 80176 | 0.12 | 4.45 | 4.454 | 4.256 | 4.281 | 4.525 | 4.709 | −0.182 | 0.166 |
| 219692_at | NM_024507 | KREMEN2 | 79412 | −0.1 | 4.77 | 5.004 | 4.713 | 4.74 | 5.021 | 4.507 | −0.16 | −0.123 |
| 219710_at | NM_024577 | SH3TC2 | 79628 | 0.721 | 2.78 | 2.991 | 2.856 | 2.84 | 2.987 | 2.85 | −0.037 | 0.034 |
| 239742_at | NM_030567 | PRR7 | 80758 | 0.379 | 5.33 | 5.489 | 5.537 | 5.394 | 6.025 | 6.008 | 0.058 | 0.608 |
| 219758_at | NM_024926 | TTC26 | 79989 | −0.52 | 4.83 | 5.148 | 4.814 | 4.912 | 4.534 | 4.376 | −0.128 | −0.535 |
| 219783_at | NM_017877 | C2orf18 | 54978 | −0.23 | 5.96 | 6.228 | 6.176 | 6.114 | 5.917 | 6.127 | 0.052 | −0.071 |
| 219784_at | NM_024735 | FBXO31 | 79791 | 0.329 | 4.88 | 4.674 | 5.016 | 4.971 | 5.186 | 5.245 | 0.218 | 0.44 |
| 219785_s_at | NM_024735 | FBXO31 | 79791 | 0.355 | 4.87 | 5.016 | 5.365 | 5.408 | 5.709 | 5.15 | 0.442 | 0.485 |
| 219794_at | NM_018289 | VPS53 | 55275 | −0.12 | 3.14 | 3.115 | 3.263 | 3.079 | 3.106 | 3.085 | 0.045 | −0.031 |
| 219801_at | NM_030580 | ZNF34 | 80778 | 0.238 | 4.25 | 3.974 | 4.164 | 4.212 | 4.693 | 4.856 | 0.076 | 0.662 |
| 219816_s_at | NM_018107 | RBM23 | 55147 | −0.84 | 7.23 | 7.215 | 7.245 | 7.227 | 6.735 | 6.736 | 0.014 | −0.487 |
| 219830_at | NM_030665 | RAI1 | 10743 | 0.25 | 3.1 | 3.018 | 3.099 | 3.245 | 3.149 | 3.24 | 0.111 | 0.134 |
| 239831_at | NM_016508 | CDKL3 | 51265 | −0.38 | 4.71 | 4.778 | 4.976 | 4.831 | 5.651 | 5.392 | 0.159 | 0.777 |
| 219842_at | NM_019087 | ARL15 | 54622 | 0.095 | 3.5 | 3.385 | 3.448 | 3.819 | 3.787 | 3.386 | 0.188 | 0.142 |
| 219862_s_at | NM_012336 | NARF | 26502 | −0.13 | 7.4 | 7.464 | 7.341 | 7.386 | 7.477 | 7.461 | −0.067 | 0.038 |
| 219899_x_at | NM_014434 | NDOR1 | 27158 | 0.31 | 3.31 | 3.513 | 3.802 | 3.401 | 3.642 | 3.481 | 0.188 | 0.148 |
| 219901_at | NM_018351 | FGD6 | 55785 | −0.13 | 3.81 | 3.56 | 3.892 | 3.818 | 3.626 | 3.204 | 0.171 | −0.269 |
| 219907_at | NM_005653 | FRS3 | 10817 | −0.11 | 3.17 | 2.741 | 2.991 | 2.913 | 3.018 | 3.026 | −0.002 | 0.068 |
| 219940_s_at | NM_018386 | PCID2 | 55795 | 0.13 | 6.94 | 6.935 | 6.949 | 6.858 | 6.876 | 6.868 | −0.034 | −0.066 |
| 219944_at | NM_024692 | CLIP4 | 79745 | 0.558 | 4.16 | 3.782 | 3.952 | 3.782 | 4.121 | 4.1 | −0.104 | 0.139 |
| 220002_at | NM_018012 | KIF26B | 55083 | 0.145 | 3.11 | 3.13 | 3.095 | 2.968 | 3.184 | 3.226 | −0.089 | 0.085 |
| 220007_at | NM_024770 | METTL8 | 79828 | 0.302 | 5.82 | 5.91 | 6.089 | 6.13 | 5.919 | 5.778 | 0.244 | −0.017 |
| 220020_at | NM_022098 | XPNPEP3 | 63929 | 0.126 | 5.09 | 5.062 | 5.316 | 5.329 | 5.252 | 5.302 | 0.247 | 0.202 |
| 220024_s_at | NM_020956 | PRX | 57716 | 0.135 | 3.51 | 3.327 | 3.225 | 3.216 | 3.75 | 3.332 | −0.2 | 0.12 |
| 220043_s_at | NM_005929 | MFI2 | 4241 | 0.158 | 2.79 | 2.962 | 3.083 | 2.941 | 3.004 | 2.835 | 0.134 | 0.041 |
| 220046_s_at | NM_020307 | CCNL1 | 57018 | 0.124 | 7.37 | 7.286 | 7.489 | 7.37 | 7.711 | 7.748 | 0.101 | 0.401 |
| 220103_s_at | NM_016067 | MRPS18C | 51023 | −0.21 | 3.14 | 3.279 | 3.207 | 3.173 | 3.215 | 3.207 | −0.018 | 0.003 |
| 220114_s_at | NM_017564 | STAB2 | 55576 | −0.07 | 3.23 | 3.282 | 3.318 | 3.126 | 3.174 | 3.231 | −0.034 | −0.053 |
| 220166_at | NM_020348 | CNNM1 | 26507 | 0.095 | 3.46 | 3.386 | 3.442 | 3.23 | 3.374 | 3.446 | −0.089 | −0.015 |
| 220172_at | NM_025000 | C2orf37 | 80067 | 0.142 | 4.61 | 4.125 | 4.134 | 3.948 | 3.938 | 4.083 | −0.326 | −0.356 |
| 220208_at | NM_017587 | ADAWTS13 | 11093 | −0.08 | 3.22 | 3.368 | 3.701 | 3.43 | 3.403 | 3.564 | 0.273 | 0.191 |
| 220227_at | NM_024883 | CDH4 | 1002 | 1.303 | 2.87 | 2.738 | 2.678 | 2.768 | 2.676 | 2.76 | −0.081 | −0.086 |
| 220228_at | AB030653 | AP4E1 | 23431 | 0.029 | 2.99 | 2.836 | 2.83 | 2.632 | 2.92 | 2.952 | −0.182 | 0.023 |
| 220229_s_at | NM_007347 | AP4E1 | 23431 | 0.016 | 3.53 | 3.783 | 3.704 | 3.481 | 3.589 | 3.508 | −0.063 | −0.107 |
| 220248_x_at | NM_018839 | NSFL1C | 55968 | 0.064 | 7.31 | 7.394 | 7.305 | 7.302 | 7.458 | 7.469 | −0.05 | 0.11 |
| 220253_s_at | NM_013437 | LRP12 | 29967 | −0.13 | 3.51 | 3.543 | 3.428 | 3.804 | 3.707 | 3.868 | 0.09 [symbol] | |
| 220254_at | NM_013437 | LRP12 | 29967 | −0.01 | 4.14 | 3.559 | 3.672 | 4.068 | 3.526 | 3.864 | 0.01 [symbol] | |
| 220271_x_at | NM_022785 | EFCAB6 | 64800 | −0.02 | 3.09 | 3.211 | 2.94 | 3.151 | 3.291 | 2.951 | −0.10 [symbol] | |
| 220312_at | NM_017708 | FAM83E | 54854 | 0.09 | 3 | 3.087 | 2.792 | 2.988 | 2.715 | 2.913 | −0.15 [symbol] | |
| 220329_s_at | NM_017909 | RMND1 | 55005 | 0.144 | 8.36 | 8.645 | 8.609 | 8.533 | 8.343 | 8.189 | 0.06 [symbol] | |
| 220349_s_at | NM_022759 | FLJ21865 | 64772 | −0.24 | 4.34 | 4.019 | 4.318 | 4.516 | 4.188 | 4.193 | 0.2 [symbol] | |
| 220395_at | NM_018602 | DNAJA4 | 55466 | −0.18 | 3.93 | 3.517 | 3.804 | 3.706 | 3.793 | 3.828 | 0.0 [symbol] | |
| 220434_at | NM_024876 | ADCK4 | 79934 | −0.07 | 3.09 | 3.1 | 3.072 | 3.346 | 3.183 | 3.199 | 0.11 [symbol] | |
| 220439_at | NM_024892 | RIN3 | 79890 | −0.05 | 3.12 | 3.03 | 3.038 | 3.003 | 2.992 | 3.021 | −0.05 [symbol] | |
| 220546_at | NM_024891 | MLL | 4297 | 0.137 | 3.08 | 3.146 | 3.13 | 3.029 | 3.22 | 3.153 | −0.03 [symbol] | |
| 220588_at | NM_017843 | BCAS4 | 55653 | 0.04 | 6.52 | 6.17 | 6.303 | 6.332 | 6.474 | 6.397 | −0.02 [symbol] | |
| 220610_s_at | NM_006309 | LRRFIP2 | 9209 | 0.504 | 5.75 | 5.934 | 5.963 | 5.998 | 6.074 | 6.181 | 0.14 [symbol] | |
| 220688_s_at | NM_016183 | MRTO4 | 51154 | 0.241 | 7.8 | 7.904 | 7.966 | 7.99 | 7.763 | 7.621 | 0.12 [symbol] | |
| 220731_s_at | NM_018090 | NECAP2 | 55707 | −0.21 | 5.01 | 5.19 | 5.196 | 5.068 | 5.49 | 5.574 | 0.03 [symbol] | |
| 220744_s_at | NM_018262 | IFT122 | 55764 | 0.282 | 5.93 | 6.031 | 5.733 | 5.913 | 6.41 | 6.623 | −0.15 [symbol] | |
| 220801_s_at | NM_016527 | HAO2 | 51179 | −0.26 | 2.83 | 2.846 | 2.644 | 2.91 | 2.829 | 2.785 | −0.06 [symbol] | |
| 220947_s_at | NM_015527 | TBC1D10B | 26000 | −0.15 | 5.35 | 5.393 | 5.328 | 5.303 | 5.403 | 5.604 | −0.05 [symbol] | |
| 220973_s_at | NM_030974 | SHARPIN | 81858 | 0.108 | 5.68 | 5.649 | 5.867 | 5.61 | 5.759 | 5.735 | 0.07 [symbol] | |
| 220986_s_at | NM_030953 | TIGD6 | 81789 | −0.24 | 3.14 | 3.055 | 3.109 | 3.005 | 3.351 | 3.274 | −0.04 [symbol] | |
| 221037_s_at | NM_031291 | SLC2SA31 | 83447 | 0.105 | 2.49 | 2.535 | 2.465 | 2.641 | 2.394 | 2.47 | 0.03 [symbol] | |
| 221049_s_at | NM_013274 | POLL | 27343 | −0.26 | 4.62 | 4.387 | 4.443 | 4.523 | 4.634 | 4.519 | −0.0 [symbol] | |
| 221206_at | NM_024521 | PMS2 /// PMS2CL | 441194 /// 5395 | 0.063 | 5.58 | 5.633 | 5.7 | 5.605 | 5.698 | 5.583 | 0.04 [symbol] | |
| 221211_s_at | NM_020152 | C21orf7 | 56911 | 0.041 | 3.06 | 2.938 | 2.962 | 2.984 | 3.004 | 2.856 | −0.02 [symbol] | |
| 221290_s_at | NM_016473 | MUM1 | 84939 | 0.432 | 3.39 | 3.602 | 3.511 | 3.447 | 3.474 | 3.356 | −0.01 [symbol] | |
| 221307_at | NM_014592 | KCNIP1 | 30820 | 0.165 | 3.23 | 3.366 | 3.333 | 3.177 | 3.23 | 3.205 | −0.04 [symbol] | |
| 221335_x_at | NM_019108 | C19orf61 | 56006 | −0.25 | 4.34 | 4.433 | 4.325 | 4.423 | 4.519 | 3.996 | −0.0 [symbol] | |
| 221438_s_at | NM_031275 | TEX12 | 56158 | −0.06 | 2.56 | 2.809 | 2.618 | 2.849 | 2.552 | 2.418 | 0.046 | −0.202 |
| 221455_s_at | NM_030753 | WNT3 | 7473 | 0.155 | 3.02 | 2.975 | 2.8 | 2.892 | 2.817 | 3.063 | −0.153 | −0.058 |
| 221499_s_at | AK_026970 | STX16 | 8675 | −0.12 | 8.06 | 7.909 | 7.842 | 8.082 | 8.191 | 8.22 | −0.023 | 0.22 |
| 221500_s_at | BE782754 | STX16 | 8675 | 0.108 | 9.9 | 9.829 | 9.864 | 9.749 | 10.28 | 10.31 | −0.056 | 0.429 |
| 221534_at | AF073483 | C11orf68 | 83638 | 0.097 | 5.21 | 4.995 | 5.08 | 4.863 | 5.313 | 4.996 | −0.132 | 0.051 |
| 221571_at | AI721219 | TRAF3 | 7187 | 0.364 | 5.7 | 5.637 | 5.779 | 5.848 | 5.832 | 6.023 | 0.146 | 0.261 |
| 221614_s_at | BC005153 | RPH3AL | 9501 | −0.09 | 3.7 | 3.718 | 3.755 | 3.884 | 3.508 | 3.745 | 0.11 | −0.082 |
| 221619_s_at | AF189289 | MTCH1 | 23787 | −0.15 | 11.4 | 11.33 | 11.47 | 11.41 | 11.31 | 11.27 | 0.082 | −0.063 |
| 221623_at | AF229053 | BCAN | 63827 | 0.047 | 2.71 | 2.79 | 2.735 | 2.858 | 2.658 | 2.793 | 0.049 | −0.022 |
| 221638_s_at | AF008937 | STX16 | 8675 | −0.25 | 5.65 | 5.861 | 5.529 | 5.87 | 6.084 | 5.857 | −0.056 | 0.216 |
| 221676_s_at | BC002342 | CORO1C | 23603 | 0.749 | 9.15 | 9.117 | 9.007 | 9.158 | 9.373 | 9.449 | −0.05 | 0.279 |
| 221702_s_at | AF353992 | TM2D3 | 80213 | −0.27 | 8.62 | 8.511 | 8.556 | 8.479 | 8.406 | 8.403 | −0.048 | −0.161 |
| 221707_s_at | BC006116 | VPS53 | 55275 | 0.221 | 3.24 | 3.155 | 3.488 | 3.221 | 3.367 | 3.483 | 0.157 | 0.228 |
| 221809_at | AB040897 | RANBP10 | 57610 | 0.003 | 3.78 | 3.829 | 3.782 | 3.684 | 3.495 | 3.663 | −0.071 | −0.225 |
| 221814_at | BF511315 | GPR124 | 25960 | 0.203 | 3.25 | 3.234 | 3.228 | 3.302 | 3.582 | 3.483 | 0.024 | 0.291 |
| 221845_s_at | AI655698 | CLPB | 81570 | −0.08 | 6.21 | 5.945 | 5.863 | 6.027 | 6.082 | 6.066 | −0.13 | −0.001 |
| 221854_at | AI378979 | PKP1 | 5317 | 0.557 | 3.81 | 3.805 | 3.87 | 3.776 | 4.747 | 4.744 | 0.015 | 0.938 |
| 221865_at | BF969986 | C9orf91 | 203197 | 0.419 | 5.77 | 5.992 | 6.212 | 5.988 | 5.69 | 5.622 | 0.22 | −0.224 |
| 221870_at | AI417917 | EHD2 | 30846 | 0.308 | 4.74 | 4.858 | 4.499 | 4.571 | 4.298 | 4.222 | −0.267 | −0.541 |
| 221881_s_at | AI638420 | CLIC4 | 25932 | 0.419 | 6.45 | 6.614 | 6.555 | 6.377 | 6.442 | 6.308 | −0.064 | −0.155 |
| 221891_x_at | AA704004 | HSPA8 | 3312 | −0.32 | 12.1 | 12.18 | 12.11 | 12.21 | 11.65 | 11.69 | 0.025 | −0.459 |
| 221897_at | AA205660 | TRIM52 | 84851 | −0.33 | 5.32 | 5.659 | 5.905 | 5.87 | 6.654 | 6.625 | 0.396 | 1.148 |
| 221899_at | AI809961 | N4BP2L2 | 10443 | −0.21 | 7.99 | 7.919 | 8.026 | 8 | 8.131 | 8.037 | 0.058 | 0.129 |
| 221920_s_at | BE677761 | SLC25A37 | 51312 | −0.34 | 3.95 | 3.974 | 3.969 | 4.215 | 3.6 | 3.878 | 0.129 | −0.224 |
| 221926_s_at | BF196320 | IL17RC | 84818 | 0.184 | 3.68 | 3.521 | 3.412 | 3.484 | 3.691 | 3.86 | 0.154 | 0.174 |
| 221960_s_at | AI89609 | RAB2A | 5862 | 0.239 | 6.18 | 6.274 | 6.136 | 6.238 | 6.145 | 6.07 | −0.04 | −0.12 |
| 221990_at | AI948472 | PAX8 | 7849 | −0.02 | 2.75 | 2.671 | 2.63 | 3.028 | 2.947 | 2.876 | 0.119 | 0.201 |
| 221998_s_at | BF062886 | VRK3 | 51231 | 0.211 | 6.65 | 6.59 | 6.503 | 6.697 | 6.768 | 6.804 | −0.021 | 0.165 |
| 221999_at | BF062886 | VRK3 | 51231 | 0.147 | 4.33 | 3.587 | 4.276 | 4.176 | 4.082 | 4.09 | 0.267 | 0.127 |
| 222010_at | BF224073 | TCP1 | 6950 | 0.111 | 6.53 | 6.681 | 6.736 | 6.809 | 6.647 | 6.675 | 0.167 | 0.055 |
| 222011_s_at | BF224073 | TCP1 | 6950 | −0.01 | 6.52 | 6.61 | 6.523 | 6.553 | 6.163 | 6.253 | −0.029 | −0.359 |
| 222035_s_at | AI984479 | PAPOLA | 10914 | 0.056 | 10.3 | 10.38 | 10.33 | 10.37 | 10.32 | 10.28 | −0.012 | −0.06 |
| 222043_at | AI982754 | CLU | 1191 | 0.181 | 3.13 | 3.278 | 3.374 | 3.36 | 2.858 | 3.077 | 0.163 | −0.236 |
| 222154_s_at | AK002064 | LOC26010 | 26010 | 0.087 | 8.09 | 8.032 | 8.107 | 8.045 | 8.03 | 8.086 | 0.013 | −0.005 |
| 222169_x_at | N71739 | SH2D3A | 10045 | −0.16 | 3.8 | 4.117 | 4.019 | 4.058 | 3.773 | 3.933 | 0.081 | −0.105 |
| 222176_at | AK021487 | PTEN | 5728 | −0.04 | 2.95 | 3.02 | 3.081 | 3.04 | 3.163 | 2.748 | 0.074 | −0.031 |
| 222188_at | AK023069 | C9orf156 | 51531 | −0.07 | 2.84 | 3.004 | 2.783 | 2.813 | 2.866 | 2.609 | −0.124 | −0.185 |
| 222195_s_at | AK023069 | C9orf156 | 51531 | −0.03 | 6.52 | 6.62 | 6.658 | 6.382 | 6.747 | 6.776 | −0.05 | 0.192 |
| 222220_s_at | AK027245 | TSNAXIP1 | 55815 | 0.216 | 3.44 | 3.425 | 3.255 | 3.498 | 3.79 | 3.619 | −0.056 | 0.272 |
| 222231_s_at | AK025328 | LRRCS9 | 55379 | 0.084 | 9.53 | 9.35 | 9.498 | 9.432 | 8.316 | 8.423 | 0.02 | |
| 222255_at | AB046840 | PRX | 57716 | 0.061 | 2.48 | 2.484 | 2.501 | 2.446 | 2.715 | 2.552 | −0.0 | |
| 222305_at | AW975638 | HK2 | 3099 | −0.03 | 4.38 | 4.271 | 4.629 | 4.54 | 4.538 | 4.412 | 0.2 | |
| 222346_at | AI633741 | LAMA1 | 284217 | −0.11 | 3.24 | 2.951 | 2.977 | 3.087 | 3.065 | 3.165 | −0.06 | |
| 222348_at | AW971134 | MAST4 | 375449 | 0.047 | 3.91 | 3.919 | 4.267 | 3.938 | 4.53 | 4.364 | 0.19 | |
| 222353_at | AV720842 | LIMD1 | 8994 | 0.148 | 3.24 | 3.132 | 3.211 | 3.133 | 3.141 | 3.092 | −0.01 | |
| 222383_s_at | AW003512 | ALOXE3 | 59344 | 0.198 | 3.05 | 3.367 | 3.189 | 3.071 | 3.284 | 3.413 | −0.07 | |
| 31846_at | AW003733 | RHOD | 29984 | 0.383 | 8.07 | 7.97 | 8.098 | 8.119 | 8.283 | 8.305 | 0.08 | |
| 31861_at | L14754 | IGHMBP2 | 3508 | −0.39 | 5.31 | 5.165 | 5.306 | 5.165 | 5.047 | 5.184 | −9E−0 | |
| 32094_at | AB017915 | CHST3 | 9469 | 0.235 | 3.66 | 3.479 | 3.632 | 3.605 | 3.516 | 3.654 | 0.05 | |
| 33132_at | U37012 | CPSF1 | 29894 | 0.074 | 6.15 | 6.275 | 6.172 | 6.2 | 6.349 | 6.388 | −0.02 | |
| 34478_at | X79780 | RAB11B | 9230 | 0.032 | 3.04 | 3.099 | 3.269 | 3.108 | 3.25 | 3.077 | 0.12 | |
| 36865_at | AB018302 | ANGEL1 | 23357 | 0.234 | 5.61 | 5.418 | 5.667 | 5.593 | 5.89 | 5.727 | 0.11 | |
| 37005_at | D28124 | NBL1 | 4681 | −0.03 | 5.66 | 5.249 | 5.473 | 5.582 | 5.428 | 5.319 | 0.07 | |
| 37566_at | AB028968 | KIAA1045 | 23349 | −0.13 | 2.87 | 2.837 | 2.779 | 2.859 | 2.902 | 2.693 | −0.03 | |
| 37860_at | AL049942 | ZNF337 | 26152 | −0.13 | 5.37 | 5.293 | 5.295 | 5.332 | 5.227 | 5.425 | −0.01 | |
| 37872_at | AF072468 | JRK | 8629 | −0.29 | 4.69 | 4.504 | 4.642 | 4.594 | 4.66 | 4.406 | 0.01 | |
| 38269_at | AL050147 | PRKD2 | 25865 | −0.07 | 6.66 | 6.46 | 6.53 | 6.492 | 6.648 | 6.837 | −0.04 | |
| 38447_at | U08438 | ADRBK1 | 156 | −0.18 | 4.52 | 4.415 | 4.411 | 4.55 | 4.492 | 4.267 | 0.01 | |
| 38918_at | AF083105 | SOX13 | 9580 | 0.103 | 4.39 | 4.633 | 4.732 | 4.422 | 4.409 | 4.183 | 0.06 | |
| 39817_s_at | AF040105 | C6orf108 | 10591 | 0.043 | 8.25 | 8.273 | 8.237 | 8.401 | 8.52 | 8.544 | 0.0 | |
| 40148_at | U62325 | APBB2 | 323 | 1.113 | 6.62 | 6.499 | 6.165 | 6.273 | 7.216 | 7.059 | −0.3 | |
| 40273_at | AA485440 | SPHK2 | 56848 | 0.223 | 4.42 | 4.521 | 4.616 | 4.618 | 4.415 | 4.464 | 0.14 | |
| 41220_at | AB023208 | 10-Sep | 10801 | −0.26 | 10.9 | 10.79 | 10.82 | 10.83 | 10.69 | 10.72 | −0.01 | |
| 41657_at | AF035625 | STK11 | 6794 | 0.073 | 4.08 | 3.902 | 4.015 | 4.259 | 4.272 | 4.501 | 0.14 | |
| 41660_at | AL031588 | CELSR1 | 9620 | 0.04 | 5.55 | 5.765 | 5.658 | 5.702 | 5.931 | 5.075 | 0.02 | |
| 44696_at | AA915989 | TBC1D13 | 54662 | −0.19 | 5.79 | 5.748 | 5.931 | 6.137 | 5.338 | 5.457 | 0.263 | −0.374 |
| 45297_at | AI417917 | EHD2 | 30846 | 0.234 | 3.53 | 3.497 | 3.454 | 3.316 | 3.316 | 3.775 | −0.129 | 0.031 |
| 47530_at | AA748492 | C9orf156 | 51531 | 0.08 | 6.34 | 6.279 | 6.329 | 6.315 | 6.375 | 6.373 | 0.014 | 0.066 |
| 53987_at | AL041852 | RANBP10 | 57610 | −0.09 | 4.25 | 4.223 | 4.22 | 4.447 | 4.38 | 4.258 | 0.097 | 0.082 |
| 54037_at | AL041451 | HPS4 | 89781 | −0.19 | 3.76 | 3.905 | 3.943 | 4.075 | 3.474 | 3.472 | 0.174 | −0.362 |
| 60471_at | AA625133 | RIN3 | 79890 | 0.368 | 3.95 | 3.642 | 3.945 | 3.583 | 4.101 | 3.964 | −0.033 | 0.235 |
| 64440_at | AI560217 | IL17RC | 84818 | −0.27 | 4.61 | 4.47 | 4.559 | 4.466 | 4.504 | 4.243 | −0.029 | −0.168 |
| 65493_at | AA555088 | HEATR6 | 63897 | −0.01 | 9.28 | 9.41 | 9.523 | 9.445 | 9.621 | 9.535 | 0.139 | 0.233 |
| 65635_at | AL044097 | FLJ21865 | 64772 | −0.25 | 4.88 | 4.904 | 4.801 | 4.755 | 4.558 | 4.864 | −0.109 | −0.181 |
| 65718_at | AI655903 | GPR124 | 25960 | 0.168 | 3.16 | 3.195 | 3.188 | 3.381 | 3.082 | 3.127 | 0.107 | −0.073 |
| 91920_at | AI205180 | BCAN | 63827 | −0.11 | 3.63 | 3.512 | 3.389 | 3.507 | 3.672 | 3.529 | −0.123 | 0.029 |

[symbol]

indicates data missing or illegible when filed

TABLE T4A
Genes bound by HSF1 in BPLER cells at 37 degrees and
not in BPE cells after heat shock (Group A genes)

AANAT, ABCA7, ABCC5, ABLIM1, ACTN4, ACY1, ADAMTS13, ADRBK1, AFF2, AK3L1,
AKAP6, ALG10, ANAPC2, ANG, ANGEL1, ANKRD13D, ANXA4, AOF2, AP4E1, APC2, ARL15,
ATXN1, B3GALNT2, B3GNT1, BAHD1, BCAN, BMF, BRF2, BRMS1, C10ORF4, C11ORF2,
C11ORF68, C14ORF112, C17ORF68, C17ORF75, C17ORF76, C19ORF25, C19ORF33,
C19ORF57, C1ORF160, C1ORF182, C1ORF66, C20ORF19, C21ORF70, C22ORF15, C22ORF16,
C2ORF18, C2ORF37, C6ORF106, C6ORF108, C6ORF150, C8ORF37, C8ORF55, C8ORF73,
C9ORF156, C9ORF75, C9ORF91, CADPS2, CALM1, CAMTA1, CAPN12, CARD11, CBS,
CCDC115, CCDC98, CCNJL, CCT3, CCT6A, CD151, CD59, CDC73, CDK5R1, CEACAM20,
CENPA, CENPT, CES2, CHCHD6, CHD4, CHST10, CIAPIN1, CKS2, CLCF1, CLPB, CNDP2,
CNGB1, CNNM1, COASY, COL2A1, COMMD2, COPS7A, COQ4, COQ9, CPSF1, CRABP2,
CRELD1, CRY1, CSF3, CUEDC1, CYC1, CYGB, CYHR1, D2HGDH, DAPK2, DBN1, DENND3,
DHX37, DHX8, DNAJA4, DNAJB12, DPY19L4, DRAP1, DTX2, DTX4, DVL1, EARS2, EEF1D,
EFCAB2, EHD2, EIF4A2, ELL, EMILIN1, ENY2, EPHA2, ERGIC1, ESR2, ESRRA, EWSR1,
FAM26B, FAM26C, FAM53C, FAM57B, FAM62A, FAM96B, FAU, FBXO31, FBXO32, FBXO47,
FEM1B, FHL3, FLJ22374, FLJ25404, FOXK2, FRS3, FSCN1, FUT10, GABRE, GALT, GFM2,
GIPC1, GNAO1, GOLGA3, GOT1, GPC1, GPR124, GPR4, GPR56, GPT, GRIFIN, GRPR, GSDM1,
GSN, GTF2F1, GTF3C3, GUSB, HCK, HDGF, HEL308, HEMGN, HIST1H4H, HK2, HMGN4,
HMHA1, HPS4, HPSE2, HRH1, HSD17B1, HSPBP1, HSPC152, HUS1, IFNAR2, IFT122, IGF1R,
IGHMBP2, IL10RB, 1L11RA, IL17RC, IL1RAP, IL6R, IMP4, ING5, IQCE, IRF2BP2, ITGB3,
JARID2, JMJD1B, JRK, KBTBD7, KCNIP3, KHK, KIAA0090, KIAA0247, KIAA1303, KIAA1333,
KIAA1737, KIF26B, KIFC2, KLF10, KREMEN2, LAMA5, LASP1, LCE1E, LFNG, LGALS7,
LHX5, LIMD1, LMNB2, LOC653147, LRP12, LRRC27, LRRC59, LRRFIP2, LSM10, LTBP4,
LY6K, LYNX1, LZIC, MACF1, MAD1L1, MAF1, MANBA, MAP2K2, MAP3K9, MAP4K4,
MATN2, MBD4, MDH2, MEGF6, METTL9, MFI2, MFSD3, MLL, MLL2, MLX, MMP11,
MRPL16, MRPL21, MRPL24, MRPL49, MRPS18C, MRPS23, MTCH1, MXRA8, MYL6, MYL6B,
MYLK, MYLPF, MYO1D, MYST2, NANOS3, NAPRT1, NARF, NARS2, NBN, NCALD, NCOR1,
NCOR2, NDOR1, NDRG1, NDUFA12, NEIL2, NEK6, NES, NFAT5, NFIX, NFKB2, NGFR,
NGRN, NMNAT1, NMT2, NOL1, NOSIP, NOXO1, NRBP2, NSFL1C, NUDCD1, NUDCD3,
NUTF2, OPA3, OSGIN1, OXR1, PABPC1, PAPOLA, PAQR4, PARC, PARN, PARP10, PAX5,
PCCA, PCGF2, PCID2, PDCD11, PDE6C, PDGFA, PEX3, PFAS, PGK1, PKN1, PLA2G6, PLEC1,
PMPCA, PMS2, PNPLA5, PNRC2, PODXL, POLA2, POLD4, POLG, POLL, POLR2L, POLR3B,
PPM1A, PPP1R16A, PPP2R2B, PRAF2, PRDX5, PRKCDBP, PRMT5, PRR7, PRRG2, PRX, PSD,
PSMB3, PSMD3, PSPH, PTEN, PTGER1, PTK2, PTOV1, PTP4A2, PVRL4, RAB11B, RAB40C,
RALGDS, RANBP10, RANBP2, RBM23, RBM25, REXO4, RFC4, RFX2, RGNEF, RHBDD3,
RHEBL1, RHOD, RIN3, RNASE4, RNF151, RP11-529110.4 (DPCD), RPL13, RPL26L1, RPL29,
RPL35, RPL8, RPS2, RPS7, RRAD, RYR1, S100A13, S100A14, S100A16, SACM1L, SAPS1,
SCFD1, SDCCAG10, SDCCAG3, SECISBP2, SEMA7A, SEPW1, SERTAD1, SF3A3, SF3B3,
SFRS7, SH2D3A, SH3PXD2A, SHARPIN, SHC4, SHF, SHKBP1, SIRPB2, SLC22A18, SLC25A45,
SLC27A4, SLC2A1, SLC39A4, SLC41A3, SLC43A2, SLC9A1, SLURP1, SNX3, SORCS2, SOX13,
SPECC1, SPG7, SPSB1, SSNA1, SSPO, STAB2, STK40, STX16, STX18, STYXL1, SUNC1,
SUSD1, SYNE2, SYNJ2BP, TAGAP, TBC1D10B, TBC1D13, TBL3, TEAD1, TESSP5, THAP11,
TIAL1, TIGD6, TINP1, TM7SF2, TM9SF4, TMED3, TNPO3, TNRC18, TRAF3, TRAPPC3,
TRIB3, TRIM41, TRIM47, TRIM52, TRIM7, TSNARE1, TSNAXIP1, TSPAN4, TTBK1, TTC26,
TTC7B, TTLL13, TYW1, UBE2D3, UBE2I, UBE2O, UBL7, UHRF1, UNC13D, UPP1, USP30,
UTP11L, VAV1, VEZT, VIP, VPS53, VRK3, WBP2, WDR45, WDR67, WNK2, XKR4, YIF1B,
ZBTB1, ZBTB25, ZC3H3, ZCCHC2, ZDHHC20, ZFPL1, ZNF180, ZNF207, ZNF213, ZNF250,
ZNF34, ZNF467, ZNF473, ZNF704, ZNHIT2, ZSCAN22.

TABLE T4B
Genes bound by HSF1 in BPLER cells at 37 degrees and in
BPE cells or HME cells after heat shock (Group B genes)

ABHD3, ACOT7, ADC, ADCK4, AGBL1, AHSA1, ALDH3B1, ALG14, ALOXE3, APBB2, APP,
ARHGEF16, ASAH3L, ATF3, ATP2C1, ATP6V1A, AZIN1, BAG3, BAGE, BAGE2, BAGE3,
BAGE4, BAGE5, BAIAP2, BANF1, BCAS4, BCL10, BMP7, BRUNOL4, BTBD11, C10ORF116,
C10ORF54, C14ORF133, C14ORF43, C17ORF67, C18ORF25, C18ORF55, C19ORF6, C1ORF172,
C20ORF117, C20ORF135, C21ORF7, C22ORF9, C2ORF42, C2ORF7, C6ORF211, C9ORF3, CA12,
CACYBP, CAP2, CARS, CAV2, CBX3, CCDC109A, CCDC117, CCDC57, CCDC97, CCNL1,
CCT4, CCT5, CCT7, CCT8, CDC25B, CDC42EP4, CDH23, CDH4, CDK3, CDKL3, CELSR1,
CENTB1, CHD3, CHORDC1, CHST3, CLIC4, CLU, CMBL, CMIP, CNN2, COPA, CORO1C,
CPA2, CPAMD8, CRYZ, CTBP2, CTNNBIP1, CUL4A, CYP24A1, DARS, DEDD2, DGKE,
DNAJA1, DNAJB1, DNAJB4, DNAJB5, DNAJB6, DNAJB7, DOCK4, DPP9, EEF1G, EFEMP1,
EGFR, EVPL, EYA1, FAM83E, FANCC, FANK1, FBLN2, FBXO15, FBXO45, FCGR2A, FGD6,
FHIT, FKBP4, FU21865, FU35767, FLJ37078, FOXP1, FUT5, FXR1, FXYD2, GCN5L2, GLA,
GL1S3, GNA15, GNAQ, GNG7, GPBP1, GPR156, GPSN2, GTPBP1, HAO2, HBCW1, HES7,
HEXIM2, HSP90AA1, HSP90AB1, HSPA4, HSPA4L, HSPA6, HSPA8, HSPB1, HSPB9, HSPD1,
HSPE1, HSPG2, HSPH1, HYPK, IDS, IFNGR2, IGF2BP2, IGFBP7, ITGB3BP, ITPKC, ITPR1,
JOSD1, KCNIP1, KCTD11, KIAA0146, KIAA0406, KIAA1026, KIAA1045, KIAA1576,
K1AA1975, KIF21A, KLHL25, KLHL26, KNTC1, KPNA1, LAMA1, LDLR, LDLRAD3,
LOC124512, LOC134145, LOC400506, LOC51252, LSM4, LYRM4, MAST4, MAT2A, MBOAT2,
MBP, METTL8, MFAP1, MGAT5, MICAL2, MKKS, MORC4, MORF4L2, MRPL18, MRPS6,
MUM1, MYO5C, NAT13, NBL1, NCSTN, NECAP2, NEDD4L, NIBP, NOP5/NOP58, NR0B2,
NTSR1, NUDC, NXN, OSBPL3, P4HA2, PAG1, PALM2, PARD6B, PARP12, PAX8, PBXIP1,
PCBD1, PDE4DIP, PDGFRB, PDXK, PDZD2, PEBP4, PGAM5, PHLDB2, PIGL, PKP1,
PLEKHA6, PLEKHG1, PMVK, POLR3E, PPP1R14C, PPP2R4, PPYR1, PRKAG2, PRKCA,
PRKCE, PRKCSH, PRKD2, PROM2, PRR12, PTGES3, PTPN1, PTPRK, PTPRN, PXDN, PXMP2,
RAB39, RAB5C, RABGAP1L, RAD51C, RAD51L1, RAI1, RANBP3, RANGAP1, RASGRF1,
RHBDD2, RORA, RPH3AL, RPL18, RPS5, RRAS, RTTN, RXRA, SAMD12, SCHIP1, SEPT9,
SERF2, SERINC4, SERPINA1, SERPINH1, SFRS10, SFRS2, SH3PXD2B, SH3TC2, SLC25A31,
SLC25A37, SLC35B2, SLC35F3, SLC45A4, SLC5A3, SLC9A11, SMS, SMYD5, SNAP23, SOS1,
SPAG1, SPATA21, SPHK2, SPIRE2, SPOCK1, SPR, SPRED2, SPTAN1, SRGAP1, SRP68, ST13,
STAT6, STIP1, STK11, STK3, STK4, STRN4, SUGT1, SYN3, SYNGR2, TAF7, TARSL2, TCP1,
TEX12, TEX2, TM2D3, TMCC1, TMEM16F, TMEM66, TMEM95, TMPRSS9, TNIK, TPD52,
TPD52L2, TPT1, TRERF1, TRIO, TRPC7, TSEN34, TTC18, TTC7A, TUT1, TYW3, UBB, UBC,
UBE2B, UBQLN1, UBTD1, USPL1, VAC14, WDR53, WDR70, WNT2, WNT3, XPNPEP3,
ZBTB38, ZC3H12A, ZCCHC17, ZFAND2A, ZNF337, ZNF526, ZNF7.

TABLE T4C
HSF1-CaSig Genes (HSF1-CSS Genes)

AANAT, ABCC5, ABHD3, ACOT7, ADAMTS13, ADAT2, ADCK4, AGBL5, AHSA1, AK3L1,
ALG10, ALOXE3, ANAPC2, ANG, ANGEL1, ANKRD13D, AOF2, APP, ASAH3L, ATF3, ATL3,
ATP2C1, ATP6V1A, ATXN1, AZIN1, B3GALNT2, B3GNT1, BAG3, BAHD1, BANF1, BCL10,
BCO2, BMF, BMS1, BRF2, BRMS1, C10orf4, C11orf2, C11orf68, C14orf112, C14orf133,
C14orf43, C17orf75, C18orf25, C18orf55, C19orf33, C19orf6, C1orf160, C1orf172, C1orf182,
C20orf19, C21orf7, C21orf70, C22orf16, C2orf37, C2orf67, C2orf7, C6orf108, C6orf150, C6orf211,
C7orf55, C8orf37, C8orf73, C9orf156, CACYBP, CALM1, CAP2, CAV2, CBX3, CCDC109A,
CCDC117, CCDC151, CCDC57, CCDC97, CCNL1, CCT3, CCT4, CCT5, CCT6A, CCT7, CCT8,
CDC73, CDK3, CDKL3, CELSR1, CENPA, CENPT, CES2, CHD3, CHORDC1, CIAPIN1, CKS2,
CLIP4, CLU, CMBL, CNN2, COASY, COMMD2, COPA, COPS7A, COQ9, CPSF1, CRELD1,
CRY1, CRYZ, CSF3, CUEDCI, CUL4A, CYHR1, CYP24A1, D2HGDH, DARS, DEDD2, DGKE,
DHX8, DNAJA1, DNAJA4, DNAJB1, DNAJB4, DNAJB5, DNAJB6, DPY19L4, DRAP1, DTX2,
DTX4, EARS2, EEF1G, EFCAB7, EIF1AD, EIF4A2, ENY2, EWSR1, FAM26B, FAM83E,
FBXO15, FBXO31, FBXO45, FBXO47, FEM1B, FGD6, FKBP4, FLJ21865, FLJ25404, FLJ35767,
FRMD8, FRS3, FUT10, FXR1, GABRE, GALT, GCN5L2, GFM2, GLA, GNA15, GOLGA3,
GPBP1, GPR4, GPR56, GPSN2, GPT, GRIFIN, GTF2F1, GTF3C3, GTPBP1, GUSB, HAO2,
HEATR6, HEL308, HIST1H4H, HMHA1, HNRNPA2B1, HNRNPH3, HPS4, HSP90AA1,
HSP90AB1, HSPA4, HSPA4L, HSPA6, HSPA8, HSPB1, HSPB9, HSPC152, HSPD1, HSPE1,
HSPH1, HUS1, HYPK, IFNGR2, IFT122, IGHMBP2, IL11RA, IMP4, ITGB3BP, JMJD1B, JMJD6,
JOSD1, JRK, KBTBD7, KCNIP3, KHK, KIAA0090, KIAA1737, KIAA1975, KIF21A, KIFC2,
KILLIN, KLC1, KLF10, KLHL25, KLHL26, KNTC1, KPNA1, KREMEN2, LASP1, LCE1E,
LMNB2, LOC124512, LOC134145, LOC26010, LOC653147, LRP12, LRRC27, LRRC59, LSM4,
LTBP4, LY6K, LZIC, MAF1, MAP2K2, MAP7D1, MAT2A, MBD4, MBOAT2, MBOAT7, MDH2,
MED23, METTL8, METTL9, MFSD3, MLL, MLL2, MLX, MMP11, MOBKL3, MORC4,
MORF4L2, MRPL16, MRPL18, MRPL21, MRPL24, MRPL49, MRPS18C, MRPS23, MRPS6,
MRTO4, MTCH1, MUL1, MUM1, MYL6, MYL6B, MYST2, N4BP2L2, NAT13, NBL1, NBN,
NCOR1, NCSTN, NDOR1, NDRG1, NDUFA12, NECAP2, NEIL2, NGRN, NIBP, NMNAT1,
NMT2, NOL1, NOP5/NOP58, NOSIP, NR0B2, NSFL1C, NUDC, NUDCD1, NUF2, NUTF2, OPA3,
OSGIN1, P4HA2, PABPC1, PAPOLA, PAQR4, PARD6B, PBLD, PCBD1, PCGF2, PCID2, PEX3,
PFAS, PGAM5, PGK1, PIGL, PLEC1, PMEPA1, PMPCA, PMVK, PNRC2, POLD4, POLG, POLL,
POLR2L, POLR3B, POLR3E, PPM1A, PRAF2, PRDX5, PRKCDBP, PRKCSH, PRKD2, PRRG2,
PSMB3, PSMD3, PSPH, PTEN, PTGES3, PTOV1, PTP4A2, PUF60, RAB11B, RAB39,
RABGAP1L, RANBP10, RANBP2, RANGAP1, RBM23, RBM25, REXO4, RHBDD2, RHBDD3,
RMND1, RNASE4, RP11-529110.4, RPH3AL, RPL13, RPL18, RPL26L1, RPL29, RPS2, RPS5,
RPS7, RRAD, RSRC2, S100A14, S100A16, SACM1L, SAPS1, SCFD1, SDCCAG10, SDCCAG3,
SECISBP2, SEPW1, SERINC4, SERPINH1, SF3A3, SFRS10, SFRS12IP1, SFRS7, SH2D3A,
SHARPIN, SHF, SLC25A45, SLC27A4, SLC45A4, SLC5A3, SLC9A1, SNAP23, SNX3, SOS1,
SPATA21, SPECC1, SPHK2, SPR, SRRD, SSPO, ST13, STAT6, STIP1, STK40, STX16, STX18,
STYXL1, SUGT1, SYNGR2, TAF7, TBC1D10B, TBC1D13, TBL3, TCP1, TCTN1, TESSP5,
TIAL1, TIGD6, TINP1, TM2D3, TM9SF4, TMED3, TMEM203, TMEM66, TMEM95, TNPO3,
TPD52, TPD52L2, TPT1, TRAF3, TRAPPC3, TRIB3, TRIM41, TRIM52, TRIM7, TSEN34,
TSNAXIP1, TSPAN4, TTC26, TYW3, UBB, UBC, UBE2B, UBE2D3, UBE2I, UBE2O, UBFD1,
UBL7, UBQLN1, UNC13D, USP30, USPL1, UTP11L, VAV1, VEZT, VIP, VRK3, WDR38,
WDR45, WDR53, XPNPEP3, ZBTB25, ZCCHC2, ZFAND2A, ZNF180, ZNF207, ZNF250,
ZNF337, ZNF34, ZNF467, ZNF473, ZNF526, ZSCAN22.

TABLE T4D
Refined HSF1-CaSig Genes (Refined HSF1-CSS Genes)

ABCC5, AHNAK2, AHSA1, AK3L1, ATP2C1, ATP6V1A, AZIN1, BAIAP2, BCL10, C6orf106,
C9orf3, CACYBP, CALM1, CARS, CBX3, CCNL1, CCT4, CCT5, CCT6A, CCT7, CDC25B,
CDC73, CENPA, CES2, CHORDC1, CHST3, CKS2, CLIC4, CLPB, COL2A1, COPA, CORO1C,
CPSF1, CRY1, CUL4A, CUX1, CYC1, DARS, DBN1, DNAJA1, DNAJB4, DNAJB6, DOCK4,
DPY19L4, DVL1, EEF1D, EGFR, EMILIN1, EWSR1, FAM96B, FXR1, GALT, GIPC1, GNG7,
GOLGA3, GPR56, HEATR6, HIST1H4H, HMGN4, HNRNPH3, HSP90AA1, HSPB1, HSPD1,
HSPG2, HSPH1, HUS1, IGFBP7, IL1RAP, IMP4, JARID2, JMJD6, JOSD1, JRK, KIAA0090,
KIAA0146, KIAA0406, KIAA1755, KLC1, KLHL25, KNTC1, KPNA1, KREMEN2, LDLR,
LMNB2, LRP12, LRRC59, LTBP4, MAP4K4, MAP7D1, MBD4, MEGF6, MICAL2, MLX,
MMP11, MRPL16, MRPL18, MTCH1, NARF, NCOR2, NDRG1, NMT2, NUDCD3, NUTF2,
OPA3, P4HA2, PAPOLA, PAQR4, PDXK, PGK1, PMEPA1, POLR3B, PRKCA, PSMB3, PTGES3,
PTK2, PUF60, PXDN, RAB5C, RBM25, REXO4, RFC4, RSRC2, SCHIP1, SF3B3, SFRS7,
SLC2A1, SLC39A4, SLC5A3, SNX3, SPOCK1, STIP1, STK3, STX16, TBC1D13, TCP1, TPD52,
TPD52L2, TSEN34, TTC26, UBE2I, UBE2O, UPP1, UTP11L, WDR67, WNT2, ZCCHC2, ZNF207,
ZNF250, ZNF337, ZNF473,

TABLE T4E
HSF1-CaSig2 Genes (composed of HSF1-Module1 and Module 2 Genes)

ABCC1, ABCC5, ABCD3, ACBD6, ACD, ACOT7, AGBL5, AHSA1, AMOTL2, ANKMY2,
AP4E1, ARID3B, ASNSD1, ATG16L1, ATL3, ATPBD4, AZIN1, BAG2, BANFI, BAX, BCAS4,
BCL2L12, BMS1, BXDC2, BZW2, C12orf30, C14orf133, C18orf25, C18orf55, C19orf62, C1orf103,
C21orf70, C2orf37, C3orf26, C6orf106, C7orf47, C9orf91, CACYBP, CAMTA1, CARS, CBX3,
CCDC117, CCDC18, CCDC58, CCDC99, CCT3, CCT4, CCT5, CCT6A, CCT7, CCT8, CD3EAP,
CD58, CD59, CDC42EP4, CDC6, CDK3, CDKN2AIPNL, CENPA, CHORDC1, CINP, CKAP2,
CKS1B, CKS2, CLEC16A, CLIC4, COPS7B, CPSF3, CSNK1A1, CTCF, CTNNBL1, CYP2R1,
CYR61, DAPK3, DCP1A, DGKE, DIDO1, DNAJA1, DNAJC21, DSN1, EARS2, EEF2, EFCAB7,
EHD2, EIF1AD, EIF2B5, EIF3H, EIF6, ELAVL1, ENTPD6, ERCC1, EXT1, FAM122B, FAM55C,
FAM83D, FAM96B, FAM98A, FKBP4, FLAD1, FLJ22222, FOXK2, FUT5, FXR1, GALNT2,
GFM2, GNG5, GPBP1, GTF2IRD1, GTF3C3, HNRNPA2B1, HNRNPA3, HNRNPF, HNRNPUL1,
HSP90AA1, HSP90AB1, HSPA4, HSPA8, HSPA9, HSPC152, HSPD1, HSPE1, HSPH1, HTATSF1,
HYPK, ICT1, IGF2BP1, IGF2BP3, IPP, IRF3, ISY1, ITGB3BP, ITGB5, JMJD6, JTB, KIAA0146,
K1AA0406, KIAA1303, KNTC1, KRT18, LAMC1, LCMT1, LIAS, LOC124512, LOC134145,
LOC144097, LOC400506, LOH12CR1, LONP1, LSM10, LSM2, LSM4, LUC7L2, MANBAL,
MAP2K2, MAP4K4, MAPRE1, MAT2A, MED1, MEPCE, METTL8, MFAP1, MLH1, MOCS2,
MORF4L2, MPHOSPH10, MRPL13, MRPL18, MRPL44, MRPL48, MRPS28, MTBP, MTDH,
MTHFD1L, MTMR12, MUM1, MYH9, MYL6, NARG1L, NAT13, NDUFV2, NKIRAS2, NKRF,
NOB1, NSUN2, NT5DC1, NUDC, NUP93, NUTF2, NXT2, ORMDL1, PAPD5, PCGF3, PGK1,
PGLS, PHTF1, PKNOX1, PLEKHH3, PMS1, PMS2, PNRC2, PPID, PRC1, PRDX6, PRKCSH,
PRMT3, PRMT5, PRNPIP, PRPF6, PSPH, PTGES3, PTK2, PTPLAD1, PXDN, RAB22A, RAB5C,
RAD51C, RAI14, RALY, RANBP3, RANGAP1, RBM23, RCC2, REXO4, RFC4, RHOF, RIC8A,
RNF169, RPL13A, RPL19, RPL22, RPL39, RPS11, RPS21, RRAS, RUVBL1, S100A13, S100A16,
SCAND1, SEC22B, SEC31A, SEC63, SECISBP2, SENP1, SEPSECS, SERPINH1, SETD5, SF3B3,
SFRS10, SFRS2, SFXN1, SH3KBP1, SHC1, SHISA5, SLC16A1, SLC35B2, SLC39A1, SLC3A2,
SLC7A5, SMARCD2, SMS, SMYD5, SNAP23, SNAP29, SNAPIN, SNX5, SNX8, SOD1, SPR,
SPRED2, SPTLC2, SRP68, ST13, STAG2, STAU1, STIP1, SUGT1, SYMPK, TAF12, TCP1, TDG,
TEAD1, TH1L, TINP1, TM2D3, TMF1, TOMM34, TPD52L2, TRAF2, TRAF3, TRIP13, TSEN34,
TTC4, TTC4, TTF2, TYW3, UBB, UBC, UBE2F, UBE2H, UBE2V1, UBFD1, UBQLN1, UBXD8,
UHRF1, USPL1, UXT, VANGL1, WDR18, WDR70, WHSC1, XPNPEP3, XPO1, YY1, ZC3H18,
ZC3HAV1, ZNF212, ZNF227, ZNF282, ZNF326, ZNF451, ZNF473, ZNHIT1, ZSCAN16.

TABLE T4F
HSF1-CaSig3

ABCA7, ACD, ACTN4, ACY1, ADCY9, ANTXR1, ASCC2, ATL3, ATP2C1, ATXN10,
B3GALNT2, B3GNT1, B4GALT1, BAG2, BLVRB, BRMS1, C15orf63, C18orf55, C1orf172,
C21orf70, C22orf15, C2orf18, C3orf64, CACNB2, CACYBP, CALM1, CARS, CCT5, CCT6A,
CCT7, CDC6, CDC73, CDH23, CENPT, CHCHD6, CIAPIN1, CKS1B, CLIC4, CNDP2, COPA,
CPSF3, CREG1, CTCF, CTNNBL1, CWC27, DGKE, DHRS12, EIF1AD, ELL, ERCC1, ESR2,
EWSR1, EXT1, FAM96B, FAM98A, FCGR2A, GALNTL1, GNAS, GOLGA3, GOT1, GTF3C3,
GTPBP1, HSPA4, HSPA6, HSPA8, HSPA9, HSPB1, ICT1, ING5, IRF3, ISY1, ITFG1, ITGB1BP1,
IVNS1ABP, JMJD6, KCNC4, KIF21A, KPNA1, LDLR, LIAS, LONP1, LRRC59, LZ1C, MAPK14,
MBD4, METTL8, MFSD3, MMP11, MMP15, MORC4, MRPL21, MRPL44, MRPS23, MRTO4,
MTDH, MTHFD1L, MUM1, MYLK3, NAA50, NCALD, NOB1, NOTCH2NL, NUDC, NUP93,
NUTF2, OAZ1, PAFAH1B1, PARD6A, PDE4DIP, PDXK, PGK1, PHF20, PLA2G15, PLA2G6,
PMPCA, PPID, PPME1, PPP1R16B, PRMT5, PSMB3, PSMD3, PTEN, PTPRS, RAD51C,
RANBP10, RANGAP1, RORA, RPH3AL, RRAD, RTTN, SF3B2, SFRS7, SIRPB2, SLC12A4,
SLC38A7, SMARCD2, SNAP29, SRP68, ST7L, STAU1, STIP1, TBC1D1, TGM2, TIAL1,
TM7SF3, TM9SF4, TP63, TRIM16, TTC7A, UBE2D3, UBE2F, UBQLN1, VPS53, VRK3, WDR53,
WNK1, WWC1, XPNPEP3, YIF1B, ZAN, ZC3HI8, ZNF451, ZNF473, AFF2, ANKRD12, BCAN,
BCO2, C10orf54, CHST3, COX16, EGFR, EPS15, FBLN1, FOXK2, FOXN3, GNAQ, GPR56,
ITPR1, JUN, KIAA0182, LPP, LRRFIP1, LTBP4, LUZP1, MACF1, MAGI1, MAP3K13, MBP,
MED23, MICAL2, NEDD4L, PDZD2, PPM1A, RAB2A, RGL1, SEC22B, SH3KBP1, SLCO3A1,
SPG7, TEAD1, TNRC18, TPD52, TRIO, TYW1, UBE21, XYLT1, ZBTB20.

TABLE T4G
All HSF1-bound Overlap with Luo dataset

AGBL5, ANAPC2, AP4E1, BTBD11, C17orf68, C9orf156, CA12, CBS, CCT3, CCT6A, CCT8,
CDC25B, CDC73, CDKL3, CLIC4, CLU, CRY1, CUX1, DTX4, ELL, ESR2, FANCC, GPR124,
GPR56, HCK, HSPD1, IL1RAP, JMJD1B, KLHL25, LOC51252, MATN2, MDH2, MED23, MLL,
MRPL49, MYLPF, NDUFA12, NEDD4L, NEIL2, NMNAT1, PARP12, PCGF2, PC1D2, PDCD11,
PDE4DIP, POLO, POLR3B, PRICKLE4, PRKCSH, PTPN1, RPL13, RPL35, SCFD1, SEMA7A,
SEPT1. SH2D3A, SH3PXD2B, SH3TC2, SHKBP1, SNAP23, SPECC1, TEAD1, TNIK, TRERF1,
TRIM52, TTC7B, UBC, UBE21, UBE2O, USP30, USPL1, VPS53, ZNF207,

TABLE T4H
BPLER Only Overlap

ANAPC2, AP4E1, C17orf68, C9orf156, CBS, CCT3, CCT6A, CDC73, CRY1, CUX1, DTX4, ELL,
ESR2, GPR124, GPR56, HCK, IL1RAP, JMJD1B, MATN2, MDH2, MED23, MLL, MRPL49,
MYLPF, NDUFA12, NEIL2, NMNAT1, PCGF2, PCID2, PDCD11, POLG, POLR3B, PRICKLE4,
RPL13, RPL35, SCFD1, SEMA7A, SEPT1, SH2D3A, SHKBP1, SPECC1, TEAD1, TRIM52,
TTC7B, UBE21, UBE2O, USP30, VPS53, ZNF207,

TABLE T4I
Shared Overlap

AGBL5, BTBD11, CAI2, CCT8, CDC25B, CDKL3, CLIC4, CLU, FANCC, HSPD1, KLHL25,
LOC51252, NEDD4L, PARP12, PDE4DIP, PRKCSH, PTPN1, SH3PXD2B, SH3TC2, SNAP23,
TNIK, TRERF1, UBC, USPL1,

TABLE T5
Publicly available gene expression datasets from breast, colon and lung carcinomas
with follow-up clinical data used for this study

| | | | | | | % | | | |
|---|---|---|---|---|---|---|---|---|---|
| | Outcome | | | ER+ | ER− | ER | Other | | |
| Dataset | Event | n = ? | Events | (n = ?) | (n = ?) | Neg | Info | GEO | Reference |
| Breast_1 | 3Y Met | 81 | 12 | 45 | 36 | 44% | x | GSE2603 | (Minn et al., 2005) |
| Breast_2 | 5Y Met | 198 | 35 | 134 | 64 | 32% | x | GSE7390 | (Desmedt et al., 2007) |
| Breast_3 | 5Y Met | 77 | 6 | 77 | 0 | 0% | x | GSE9195 | (Loi et al., 2008) |
| Breast_4 | Overall | 88 | 28 | 88 | 0 | 0% | x | GSE6532 | (Loi et al., 2007) |
| | Met | | | | | | | | |
| Breast_5 | 5Y Met | 200 | 28 | 156 | 44 | 22% | x | GSE11121 | (Schmidt et al., 2008) |
| Breast_6 | 5Y Status | 102 | 42 | 68 | 34 | 33% | x | GSE3143 | (Bild et al., 2006) |
| Breast_7 | Relapse | 286 | 107 | 209 | 77 | 27% | x | GSE2034 | (Wang et al., 2005) |
| Breast_8 | 3Y | 108 | 15 | 75 | 30 | 28% | x | X | http://cancergenome.nih.gov/ |
| | Survival | | | | | | | | |
| Breast_9 | Overall | 159 | 46 | 130 | 29 | 18% | x | GSE1456 | (Pawitan et al., 2005) |
| | Survival | | | | | | | | |
| Breast_10 | 5Y Met | 295 | 78 | 226 | 69 | 23% | x | X | (van de Vijver et al., 2002) |
| Lung_1 | 3Y | 50 | 27 | NA | NA | NA | Lung | GSE3141 | (Bild et al., 2006) |
| | Survival | | | | | | Adeno- | | |
| | | | | | | | carcinoma | | |
| Lung_2 | 3Y | 37 | 9 | NA | NA | NA | Lung | GSE19188 | (Hou et al., 2010) |
| | Survival | | | | | | Adeno- | | |
| | | | | | | | carcinoma | | |
| Colon_1 | 5Y | 95 | 58 | NA | NA | NA | x | GSE14333 | (Jorissen et al., 2009) |
| | Relapse | | | | | | | | |
| Colon_2 | 5Y | 119 | 67 | NA | NA | NA | x | GSE17538 | (Smith et al., 2010) |
| | Survival | | | | | | | | |

TABLE T6
Multivariate analysis of breast cancer-specific mortality by HSF1-
status (HSF1 high positive or low positive versus HSF1-negative).

| Models | | | | | |
|---|---|---|---|---|---|
| ER-positive, node | N | | Hazard Ratio (95% CI) | | |
| negative cases: | Cases | Endpoints | None | Low | High |
| Model¹ | 947 | 142 | 1.00 | 1.65 (1.02-2.66) | 2.41 (1.45-3.99) |
| Model² | 947 | 142 | 1.00 | 1.42 (0.88-2.31) | 1.98 (1.17-3.33) |

*CI denotes the confidence interval.
Model¹: Adjust for age at diagnosis (years).
Model²: Adjust for age at diagnosis (years), date of diagnosis (months), disease stage (I, II, III), grade (I, II, III), radiation treatement (yes, no, missing), chemotherapy and hormonal treatment (no/no, yes/no, no/yes, yes/yes, missing).

TABLE T8

| | TISSUE | | | | | | |
|---|---|---|---|---|---|---|---|
| | BREAST | | | | | | |
| | VandeVijver. | VandeVijver. | Desmedt. | Desmedt. | Schmidt. | Schmidt. | Loi_2007. |
| Dataset | stat | p.value | stat | p.value | stat | p.value | stat |
| HSF1-CaSig | 10.59556316 | 0.001133594 | 3.35197896 | 0.0671243 | 4.470804 | 0.034479 | 12.825259 |
| MEDIAN | 1.095187332 | 0.295324716 | 0.51544814 | 0.4727899 | 0.728541 | 0.393356 | 0.3690818 |
| RANDOM | | | | | | | |
| 95th percentile | 8.992320977 | 0.922410918 | 4.19370766 | 0.9495215 | 6.438233 | 0.933538 | 2.9219695 |
| RANDOM | | | | | | | |
| Individual | 0.033 | | 0.079 | | 0.096 | | 0.000 |
| Monte Carlo p- | | | | | | | |
| value (HSF1- | | | | | | | |
| CaSig vs | | | | | | | |
| RANDOM) | | | | | | | |

| | TISSUE | | | | | |
|---|---|---|---|---|---|---|
| | BREAST | | | | | |
| | Loi_2007. | | Wang.p. | | Pawitan.p. | |
| Dataset | p.value | Wang.stat | value | Pawitan.stat | value | Bild.stat |
| HSF1-CaSig | 0.000342 | 20.05993482 | 7.51E−06 | 28.7643105 | 8.17E−08 | 7.530037 |
| MEDIAN | 0.5435052 | 0.863645563 | 0.352721 | 0.89275441 | 0.34473198 | 0.970945 |
| RANDOM | | | | | | |
| 95th percentile | 0.9522965 | 6.518335621 | 0.930128 | 7.07858185 | 0.90500162 | 3.8635548 |
| RANDOM | | | | | | |
| Individual | | 0.000 | | 0.000 | | 0.002 |
| Monte Carlo p- | | | | | | |
| value (HSF1- | | | | | | |
| CaSig vs | | | | | | |
| RANDOM) | | | | | | |

| | TISSUE | | | | | | |
|---|---|---|---|---|---|---|---|
| | BREAST | | | | | LUNG | |
| | Bild.p. | Minn2. | Minn2.p. | Loi_2008. | Loi_2008. | Bild_Lung. | Bild_Lung. |
| Dataset | value | stat | value | stat | p.value | stat | p.value |
| HSF1-CaSig | 0.0061 | 0.06825 | 0.793902 | 11.091938 | 0.00086704 | 4.78724803 | 0.0286712 |
| MEDIAN | 0.3244 | 0.49276 | 0.482698 | 0.2317658 | 0.6302177 | 0.49742498 | 0.48063376 |
| RANDOM | | | | | | | |
| 95th percentile | 0.9005 | 3.88746 | 0.954378 | 3.4123587 | 0.79307685 | 3.53245717 | 0.94503425 |
| RANDOM | | | | | | | |
| Individual | | 0.803 | | 0.033 | | 0.014 | |
| Monte Carlo p- | | | | | | | |
| value (HSF1- | | | | | | | |
| CaSig vs | | | | | | | |
| RANDOM) | | | | | | | |

| | TISSUE | | | | | |
|---|---|---|---|---|---|---|
| | LUNG | | COLON | | | |
| | Hou_Lung. | Hou_Lung. | Jorissen_Colon. | Jorissen_Colon. | Smith_Colon. | Smith_Colon. |
| Dataset | stat | p.value | stat | p.value | stat | p.value |
| HSF1-CaSig | 0.50997978 | 0.47514761 | 10.65842 | 0.00109571 | 4.30056 | 0.03809992 |
| MEDIAN | 0.43955795 | 0.50733589 | 1.578549 | 0.20896976 | 0.2389 | 0.62500466 |
| RANDOM | | | | | | |
| 95th | 3.3805951 | 0.95879899 | 5.833404 | 0.88622026 | 1.81335 | 0.96639823 |
| percentile | | | | | | |
| RANDOM | | | | | | |
| Individual | 0.463 | | 0.007 | | 0.001 | |
| Monte Carlo | | | | | | |
| p-value | | | | | | |
| (HSF1- | | | | | | |
| CaSig vs | | | | | | |
| RANDOM) | | | | | | |

TABLE T9

| | | HSF1- | HSF1- | HSF1- |
|---|---|---|---|---|
| Dataset | Reference | CaSig | CaSig2 | CaSig3 |
| Breast_1 | (Pawitan et al., 2005) | 0.0001 | 0.0028 | * |
| Breast_2 | (van de Vijver et al., 2002) | 0.0057 | <0.0001 | 0.0016 |
| Breast_3 | (Wang et al., 2005) | 0.0027 | 0.0221 | 0.0015 |
| Breast_4 | (Bild et al., 2006) | 0.0047 | 0.0092 | 0.0079 |
| Breast_5 | TCGA: | 0.0001 | 0.0453 | 0.0052 |
| | http://cancergenome.nih.gov/ | | | |
| Breast_6 | (Schmidt et al., 2008) | 0.0124 | 0.0093 | 0.0003 |
| Breast_7 | (Loi et al., 2007) | 0.0144 | 0.0005 | 0.0421 |
| Breast_8 | (Loi et al., 2008) | 0.0134 | 0.0166 | 0.0005 |
| Breast_9 | (Desmedt et al., 2007) | 0.0058 | 0.0115 | 0.1008 |
| Breast_10 | (Minn et al., 2005) | 0.4475 | 0.1472 | 0.0017 |
| Lung_1 | (Bild et al., 2006) | 0.0489 | 0.0052 | 0.0014 |
| Lung_2 | (Hou et al., 2010) | 0.0099 | 0.8487 | * |
| Colon_1 | (Jorissen et al., 2009) | 0.0001 | <0.0001 | * |
| Colon_2 | (Smith et al., 2010) | 0.0473 | 0.1482 | 0.0006 |

* Used as training dataset for HSF1-CaSig3

We claim:

1. A method of diagnosing cancer in a subject comprising the steps of: determining the level of Heat Shock Factor-1 (HSF1) activity in a sample obtained from the subject, wherein increased HSF1 activity in the sample is indicative that the subject has cancer, and wherein determining the level of HSF1 activity comprises measuring the level of a gene expression product of at least one HSF1 cancer signature set (CSS) gene listed in Table T4C.

2. The method of claim 1, wherein the method comprises comparing the level of a gene expression product of the at least one HSF1 CSS gene listed in Table T4C with a control level, wherein a greater level in the sample as compared with the control level is indicative that the subject has cancer.

3. The method of claim 1, wherein the cancer is a cancer in situ (CIS).

4. (canceled)

5. The method of claim 1, wherein the sample comprises breast, lung, colon, prostate, pancreas, cervical, or nerve sheath tissue.

6. The method of claim 1, wherein the sample comprises breast tissue and the cancer is ductal carcinoma in situ (DCIS).

7.-17. (canceled)

18. A method for providing prognostic information relating to a tumor, the method comprising: determining the level of HSF1 activity in a tumor sample from a subject in need of tumor prognosis, wherein if the level of HSF1 activity is increased, the subject is considered to have a poor prognosis, and wherein determining the level of HSF1 activity comprises determining the level of a gene expression product of at least one HSF1 cancer signature set (CSS) gene listed in Table T4C.

19. The method of claim 18, the method comprising steps of: (a) determining the level of a gene expression product of the at least one HSF1 CSS gene in the sample; and (b) comparing the level with a control level, wherein if the level determined in (a) is greater than the control level, the subject is considered to have a poor prognosis.

20. A method for providing treatment-specific predictive information relating to a tumor, the method comprising: determining the level of HSF1 activity in a tumor sample from a subject in need of tumor prognosis, wherein the level of HSF1 activity correlates with tumor sensitivity or resistance to a treatment, thereby providing treatment-specific predictive information, wherein determining the level of HSF1 activity comprises measuring the level of a gene expression product of at least one HSF1 cancer signature set (CSS) gene listed in Table T4C.

21. The method of claim 20, the method comprising steps of: (a) determining the level of HSF1 activity in the sample; and (b) comparing the level with a control level, wherein if the level of HSF1 activity is greater than the control level, the tumor has an increased likelihood of being resistant to hormonal therapy.

22. The method of claim 20, the method comprising steps of: (a) determining the level of HSF1 activity in the sample; (b) comparing the level with a control level, wherein if the level of HSF1 activity determined in (a) is greater than the control level, the tumor has an increased likelihood of being sensitive to proteostasis modulator therapy.

23.-31. (canceled)

32. The method of claim 18, wherein the tumor is a Stage I tumor.

33. The method of claim 18, wherein the tumor is a breast, lung, colon, prostate, cervical, pancreatic, or nerve sheath tumor.

34.-56. (canceled)

57. The method of claim 18, wherein determining the level of HSF1 activity comprises determining the level of a gene expression product of at least one HSF1-CSS gene whose expression is increased by at least 1.2-fold in cancer cells as compared with non-transformed control cells not subjected to heat shock.

58. The method of claim 57, wherein determining the level of HSF1 activity comprises determining the level of a gene expression product of at least 2, 3, 4, 5, 10, 15, 20, 25, 30, 40, 50, 60, 70, 80, 90, or 100 of said HSF1-CSS genes.

59.-124. (canceled)

125. A method of identifying a candidate modulator of HSF1 cancer-related activity comprising steps of:
(a) contacting a cell that expresses HSF1 with a test agent;
(b) measuring the level of an HSF1 cancer-related activity exhibited by the cell; and
(c) determining whether the test agent modulates the HSF1 cancer-related activity, wherein a difference in the level of the HSF1 cancer-related activity in the presence of the test agent as compared to the level in the absence of the test agent identifies the agent as a candidate modulator of HSF1 cancer-related activity.

126. The method of claim 125, wherein measuring the level of an HSF cancer-related activity comprises measuring binding of HSF1 to a regulatory region of an HSF1-CP gene, Group A gene, HSF1-CSS gene, HSF1-CaSig2 gene, HSF1-CaSig3 gene, refined HSF1-CSS gene, Module 1 gene, Module 2 gene, Module 3 gene, Module 4 gene, or Module 5 gene or measuring expression of an HSF1-CP gene, Group A gene, Group B gene, HSF1-CSS gene, refined HSF1-CSS gene, Module 1 gene, Module 2 gene, Module 3 gene, Module 4 gene, or Module 5 gene, wherein the gene is more highly bound by HSF1 in cancer cells than in heat shocked non-transformed control cells.

127. (canceled)

128. The method of claim 125, wherein measuring the level of an HSF cancer-related activity comprises measuring expression of at least 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, Group B genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes, wherein at least one of the genes is more highly bound by HSF1 in cancer cells than in non-cancer control cells, wherein the test agent is identified as a candidate modulator of HSF1 cancer-related activity if the presence of the test agent coordinately affects expression of at least two genes that are coordinately regulated by HSF1 in cancer cells.

129.-131. (canceled)

132. The method of claim 125, comprising administering a candidate HSF1 modulator to a non-human animal that serves as a cancer model.

133. A collection comprising reagents suitable for assessing expression of at least 2, 3, 4, 5, 10, 15, 20, 25, 30, 40, 50, 60, 70, 80, 90, 100, 150, 200, 250, 300, 350, 400, 450 or at least 10%, 20%, 30%, 40%, 50%, 60%, 70%, 80%, 90%, 95%, 99%, or all HSF1-CP genes, Group A genes, Group B genes, HSF1-CSS genes, HSF1-CaSig2 genes, HSF1-CaSig3 genes, refined HSF1-CSS genes, Module 1 genes, Module 2 genes, Module 3 genes, Module 4 genes, or Module 5 genes.

134. The collection of claim 133, wherein the reagents comprise probes, primers, or binding agents.

135.-144. (canceled)
